(19) United States
(12) Patent Application Publication
(10) Pub. No.: US 2024/0315130 A1
(43) Pub. Date: Sep. 19, 2024

(54) HETEROCYCLIC COMPOUND AND ORGANIC LIGHT-EMITTING DEVICE COMPRISING SAME

(71) Applicant: LT MATERIALS CO., LTD., Gyeonggi-do (KR)

(72) Inventors: Nam Jin LEE, Yongin-si (KR); Dong Jin LEE, Yongin-si (KR); Won Jang JEONG, Yongin-si (KR); Dong Jun KIM, Yongin-si (KR)

(73) Assignee: LT MATERIALS CO., LTD., Yongin-si, Gyeonggi-do (KR)

(21) Appl. No.: 18/276,297

(22) PCT Filed: Feb. 24, 2022
(86) PCT No.: PCT/KR2022/002685

§ 371 Date: Aug. 8, 2023

(30) Foreign Application Priority Data
Mar. 31, 2021 (KR) 10-2021-0041960

(51) Int. Cl. H10K 85/60; C07D 219/08; C07D 405/10; C07D 405/12; C07D 409/04; C07D 409/10; C07D 409/12; C09K 11/06; H10K 50/11; H10K 50/15; H10K 50/18

(52) U.S. Cl. CPC H10K 85/636; C07D 219/08; C07D 405/10; C07D 405/12; C07D 409/04; C07D 409/10; C07D 409/12; C09K 11/06; H10K 85/633; C09K 2211/1018; H10K 50/11; H10K 50/15; H10K 50/181; H10K 85/615; H10K 85/622; H10K 85/626; H10K 85/6572; H10K 85/6574; H10K 85/6576

(57) ABSTRACT

The present disclosure relates to a heterocyclic compound represented by Chemical Formula 1, an organic light emitting device comprising the same, a manufacturing method thereof, and a composition for organic material layer.

### TECHNICAL FIELD

This application claims priority to and the benefits of Korean Patent Application No. 10-2021-0041960, filed with the Korean Intellectual Property Office on Mar. 31, 2021, the entire contents of which are incorporated herein by reference.

The present disclosure relates to a heterocyclic compound and an organic light emitting device comprising the same.

### BACKGROUND ART

An organic light emitting device is a type of self-emission type display device, and there are advantages in that it not only has a wide viewing angle and excellent contrast, but also has fast response speed.

The organic light emitting device has a structure in which an organic thin film is disposed between two electrodes. When a voltage is applied to the organic light emitting device having such a structure, electrons and holes injected from the two electrodes combine in the organic thin film to form a pair, and then emit light while being disappeared. The organic thin film may be composed of a single layer or multiple layers as needed.

A material of the organic thin film may have a light emitting function as needed. For example, as the organic thin film material, a compound capable of forming the light emitting layer by itself may be used, or a compound capable of serving as a host or dopant of the host-dopant based light emitting layer may also be used. In addition, compounds capable of performing the roles of hole injection, hole transport, electron blocking, hole blocking, electron transport, electron injection, etc. may also be used as the material of the organic thin film.

In order to improve the performance, lifetime, or efficiency of the organic light emitting device, the development of the material of the organic thin film is continuously required.

### PRIOR ART DOCUMENTS

### Patent Documents

(Patent Document 1) U.S. Pat. No. 4,356,429

### DISCLOSURE

### Technical Problem

An object of the present disclosure is to provide a heterocyclic compound, an organic light emitting device comprising the same, a manufacturing method thereof, and a composition for an organic material layer.

### Technical Solution

In order to achieve the above object, the present disclosure provides a heterocyclic compound represented by

Chemical Formula 1 below.

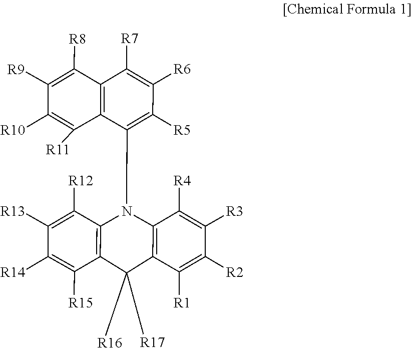

In Chemical Formula 1,
R1 to R17 are the same as or different from each other, each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60heterocycloalkyl group; a substituted or unsubstituted C6 to C60 aryl group; a substituted or unsubstituted C2 to C60 heteroaryl group; —P(═O)R101R102; —SiR101R102R103; and a group represented by Chemical Formula 2 below, or two or more groups adjacent to each other are bonded to each other to form a substituted or unsubstituted C6 to C60 aromatic hydrocarbon ring or a substituted or unsubstituted C2 to C60 heterocycle, wherein R101, R102, and R103 are the same as or different from each other, and each independently a substituted or unsubstituted C1 to C60 alkyl group; a substituted or unsubstituted C6 to C60 aryl group; ora substituted or unsubstituted C2 to C60 heteroaryl group,
at least one of R1 to R4 and R12 to R15 is a substituted or unsubstituted C6 to C60 aryl group; a substituted or unsubstituted C2 to C60 heteroaryl group; or a group represented by Chemical Formula 2 below,
at least one of R5 to R11 is a substituted or unsubstituted C6 to C60 aryl group; a substituted or unsubstituted C2 to C60 heteroaryl group; or a group represented by Chemical Formula 2 below,

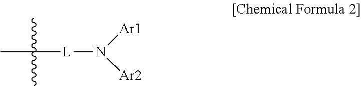

In Chemical Formula 2,
Ar1 and Ar2 are the same as or different from each other, and each independently a substituted or unsubstituted C6 to C60 aryl group; or a substituted or unsubstituted C2 to C60heteroaryl group, and
L is a direct bond; a substituted or unsubstituted C6 to C60 arylene group; or a substituted or unsubstituted C2 to C60heteroarylene group.

Furthermore, the present disclosure provides an organic light emitting device comprising:
a first electrode;
a second electrode provided to face the first electrode; and
one or more organic material layers provided between the first electrode and the second electrode,
wherein one or more layers of the organic material layers comprise the heterocyclic compound represented by Chemical Formula 1 above.

### Advantageous Effects

The compound described in the present specification can be used as a material of an organic material layer of an organic light emitting device. The compound can serve as a hole injection layer material, an electron blocking layer material, a hole transport layer material, a light emitting layer material, an electron transport layer material, a hole blocking layer material, an electron injection layer material, etc. in the organic light emitting device. In particular, the compound can be used as a hole transport layer material, an electron blocking layer material, or a light emitting layer material of the organic light emitting device.

Specifically, the compound can be used alone as a light emitting material, or can be used as a host material or a dopant material of the light emitting layer. When the compound represented by Chemical Formula 1 above is used in the organic material layer, the compound can lower the driving voltage of the organic light device, improve the luminous efficiency thereof, and improve the lifetime properties thereof.

In particular, in the heterocyclic compound represented by Chemical Formula 1 above of the present disclosure, the LUMO is delocalized to improve the stability and mobility of electrons, thereby exhibiting the effect of improving the lifetime of the organic electroluminescent device.

Further, the heterocyclic compound represented by Chemical Formula 1 above of the present disclosure has a high triplet energy level (T1 level) to exhibit the effect of preventing retrogression of energy transfer from the dopant to the host and preserving triplet exciton well within the light emitting layer.

Further, the heterocyclic compound represented by Chemical Formula 1 above of the present disclosure can preserve exciton well by facilitating intramolecular charge transfer and reducing the energy gap between the singlet energy level (S1) and the triplet energy level (T1).

### DESCRIPTION OF DRAWINGS

FIGS. 1 to 3 are diagrams each schematically showing a lamination structure of an organic light emitting device according to an embodiment of the present disclosure.

### BEST MODE FOR CARRYING OUT THE INVENTION

Hereinafter, the present disclosure will be described in more detail.

In the present specification, the term “substitution” means that a hydrogen atom bonded to a carbon atom of a compound is changed to another substituent, and the position to be substituted is not limited as long as it is a position at which the hydrogen atom is substituted, that is, a position where the substituent is substitutable, and when two or more substituents are substituted, two or more substituents may be the same as or different from each other.

In the present specification, “substituted or unsubstituted” means that it is substituted or unsubstituted with one or more substituents selected from the group consisting of: deuterium; halogen; a cyano group; C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine or being unsubstituted, or being substituted with a substituent linking two or more substituents selected from among substituents illustrated above.

In the present specification, the halogen may be fluorine, chlorine, bromine, or iodine.

In the present specification, the alkyl group may include a linear or branched chain having 1 to 60 carbon atoms, and may be further substituted by other substituents. The number of carbon atoms of the alkyl group may be 1 to 60, specifically 1 to 40, and more specifically 1 to 20. Specific examples of the alkyl group may include a methyl group, an ethyl group, an n-propyl group, an isopropyl group, an n-butyl group, an isobutyl group, a tert-butyl group, a sec-butyl group, an 1-methyl-butyl group, an 1-ethyl-butyl group, an n-pentyl group, an isopentyl group, a neopentyl group, a tert-pentyl group, an n-hexyl group, an 1-methylpentyl group, a 2-methylpentyl group, a 4-methyl-2-pentyl group, a 3,3-dimethylbutyl group, a 2-ethylbutyl group, an n-heptyl group, an 1-methylhexyl group, a cyclopentylmethyl group, a cyclohexylmethyl group, an n-octyl group, a tert-octyl group, an 1-methylheptyl group, a 2-ethylhexyl group, a 2-propylpentyl group, an n-nonyl group, a 2,2-dimethylheptyl group, an 1-ethyl-propyl group, an 1,1-dimethyl-propyl group, an isohexyl group, a 2-methylpentyl group, a 4-methylhexyl group, a 5-methylhexyl group, etc., but are not limited thereto.

In the present specification, the alkenyl group may include a linear or branched chain having 2 to 60 carbon atoms, and may be further substituted by other substituents. The number of carbon atoms of the alkenyl group may be 2 to 60, specifically 2 to 40, and more specifically 2 to 20. Specific examples of the alkenyl group may include a vinyl group, an 1-propenyl group, an isopropenyl group, an 1-butenyl group, a 2-butenyl group, a 3-butenyl group, an 1-pentenyl group, a 2-pentenyl group, a 3-pentenyl group, a 3-methyl-1-butenyl group, an 1,3-butadienyl group, an allyl group, an 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stylbenyl group, a styrenyl group, etc., but are not limited thereto.

In the present specification, the alkynyl group may include a linear or branched chain having 2 to 60 carbon atoms, and may be further substituted by other substituents. The number of carbon atoms of the alkynyl group may be 2 to 60, specifically 2 to 40, and more specifically 2 to 20.

In the present specification, the alkoxy group may be a linear, branched, or cyclic chain. The number of carbon atoms of the alkoxy group is not particularly limited, but is preferably 1 to 20. Specific examples of the alkoxy group may include a methoxy group, an ethoxy group, a n-propoxy group, an isopropoxy group, an n-butoxy group, an isobutoxy group, a tert-butoxy group, a sec-butoxy group, an n-pentyloxy group, a neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group, etc., but are not limited thereto.

In the present specification, the cycloalkyl group may include a monocyclic or polycyclic ring having 3 to 60 carbon atoms, and may be further substituted by other substituents. Here, the polycyclic ring refers to a group in which a cycloalkyl group is directly connected to or condensed with other ring group. Here, although the other ring group may be a cycloalkyl group, it may also be a different type of ring group, for example, a heterocycloalkyl group, an aryl group, a heteroaryl group, or the like. The number of carbon atoms of the cycloalkyl group may be 3 to 60, specifically 3 to 40, and more specifically 5 to 20. Specific examples of the cycloalkyl group may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, etc., but are not limited thereto.

In the present specification, the heterocycloalkyl group may contain O, S, Se, N, or Si as a heteroatom, may include a monocyclic or polycyclic ring having 2 to 60 carbon atoms, and may be further substituted by other substituents. Here, the polycyclic ring refers to a group in which a heterocycloalkyl group is directly connected to or condensed with other ring group. Here, although the other ring group may be a heterocycloalkyl group, it may also be a different type of ring group, for example, a cycloalkyl group, an aryl group, a heteroaryl group, or the like. The number of carbon atoms of the heterocycloalkyl group may be 2 to 60, specifically 2 to 40, and more specifically 3 to 20.

In the present specification, the aryl group may include a monocyclic or polycyclic ring having 6 to 60 carbon atoms, and may be further substituted by other substituents. Here, the polycyclic ring means a group in which an aryl group is directly connected to or condensed with other ring group. Here, although the other ring group may be an aryl group, it may also be a different type of ring group, for example, a cycloalkyl group, a heterocycloalkyl group, a heteroaryl group, or the like. The aryl group includes a spiro group. The number of carbon atoms of the aryl group may be 6 to 60, specifically 6 to 40, and more specifically 6 to 25. Specific examples of the aryl group may include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, condensed ring groups thereof, etc., but are not limited thereto.

In the present specification, the phosphine oxide group may be represented by —P(═O)R101R102, wherein R101 and R102 may be the same as or different from each other, and may be each independently a substituent consisting of at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and the above-described examples may be applied to the aryl group. Examples of the phosphine oxide group may include a diphenylphosphine oxide group, a dinaphthylphosphine oxide group, etc., but are not limited thereto.

In the present specification, the silyl group may be a substituent which contains Si and to which the Si atom is directly connected as a radical, and may be represented by —SiR101R102R103, wherein R101 to R103 may be the same as or different from each other, and may be each independently a substituent consisting of at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but are not limited thereto.

In the present specification, the fluorenyl group may be substituted, and adjacent substituents may be bonded to each other to form a ring.

When the fluorenyl group is substituted, it may become

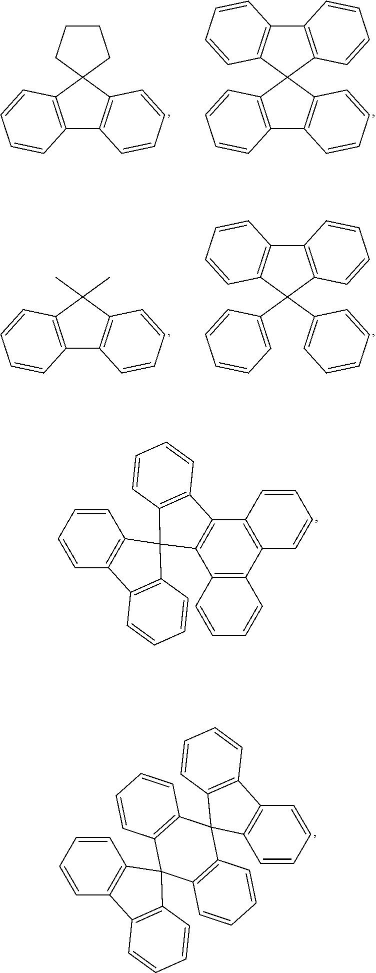

etc., but is not limited thereto.

In the present specification, the heteroaryl group may include S, O, Se, N, or Si as a heteroatom, may include a monocyclic or polycyclic ring having 2 to 60 carbon atoms, and may be further substituted by other substituents. Here, the polycyclic ring refers to a group in which a heteroaryl group is directly connected to or condensed with other ring group. Here, although the other ring group may be a heteroaryl group, it may also be a different type of ring group, for example, a cycloalkyl group, a heterocycloalkyl group, an aryl group, or the like. The number of carbon atoms of the heteroaryl group may be 2 to 60, specifically 2 to 40, and more specifically 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxinyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diazanaphthalenyl group, a triazaindenyl group, an indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilole a group, spirobi (dibenzosilole) group, a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1, 4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, etc., but are not limited thereto.

In the present specification, the amine group may be selected from the group consisting of: a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms of the amine group is not particularly limited, but is preferably 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, etc., but are not limited thereto.

In the present specification, the arylene group means the aryl group having two bonding sites, that is, a divalent group. The description of the aryl group described above may be applied except that each of these is a divalent group. Further, the heteroarylene group means the heteroarlyene group having two bonding sites, that is, a divalent group. The description of the heteroaryl group described above may be applied except that each of these is a divalent group.

In the present specification, the “adjacent” group may mean a substituent substituted on an atom directly connected to the atom in which the corresponding substituent is substituted, a substituent positioned to be sterically closest to the corresponding substituent, or another substituent substituted on the atom in which the corresponding substituent is substituted. For example, two substituents substituted at an ortho position in a benzene ring and two substituents substituted at the same carbon in an aliphatic ring may be interpreted as groups “adjacent” to each other.

In the present disclosure, “when a substituent is not indicated in a chemical formula or compound structure” means that a hydrogen atom is bonded to a carbon atom. However, since deuterium (²H) is an isotope of hydrogen, some hydrogen atoms may be deuterium.

In an embodiment of the present disclosure, “when a substituent is not indicated in a chemical formula or compound structure” may mean that all positions which can come as a substituent are hydrogen or deuterium. That is, deuterium may be an isotope of hydrogen, and some hydrogen atoms may be deuterium that is an isotope, and the content of deuterium may be 0 to 100% at this time.

In an embodiment of the present disclosure, “when a substituent is not indicated in a chemical formula or compound structure”, if deuterium is not explicitly excluded, such as “the content of deuterium of 0%”, “the content of hydrogen of 100%”, “all of the substituents being hydrogen”, etc., hydrogen and deuterium may be mixed and used in the compound.

In an embodiment of the present disclosure, in “when a substituent is not indicated in the chemical formula or compound structure”, if deuterium is not explicitly excluded as in “the content of deuterium is 0%”, “the content of hydrogen is 100%”, “the substituents are all hydrogen”, etc., hydrogen and deuterium may be mixed and used in the compound.

In an embodiment of the present disclosure, deuterium is an element having a deuteron consisting of one proton and one neutron as one of the isotopes of hydrogen as a nucleus, it may be expressed as hydrogen-2, and an element symbol may also be written as D or ²H.

In an embodiment of the present disclosure, although isotopes meaning atoms which have the same atomic number (Z), but have different mass numbers (A) have the same number of protons, they may also be interpreted as elements with different numbers of neutrons.

In an embodiment of the present disclosure, when the total number of substituents that a basic compound may have is defined as T1, and the number of specific substituents among them is defined as T2, the meaning of the content T % of the specific substituents may be defined as T2/T1×100=T %.

That is, as an example, the 20% content of deuterium in the phenyl group represented by

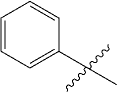 [SMILES: *c1ccccc1]

may mean when the total number of substituents that the phenyl group may have is 5 (T1 in Equation) and the number of deuterium among them is 1 (T2 in Equation). That is, it may be represented by the structural formula below that the content of deuterium in the phenyl group is 20%.

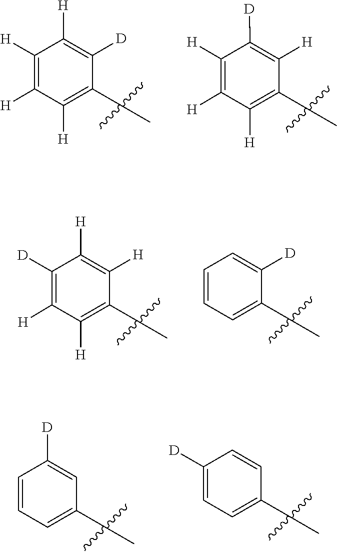 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an embodiment of the present disclosure, in the case of “a phenyl group having a deuterium content of 0%”, it may mean a phenyl group that does not contain a deuterium atom, that is, has 5 hydrogen atoms.

In the present disclosure, a C6 to C60 C6 to C60 aromatic hydrocarbon ring may mean a compound containing an aromatic ring consisting of C6 to C60 carbons and hydrogen, and examples of the C6 to C60 aromatic hydrocarbon ring may include benzene, biphenyl, terphenyl, triphenylene, naphthalene, anthracene, phenalene, phenanthrene, fluorene, pyrene, chrysene, perylene, azulene, etc., but are not limited thereto, and may include all aromatic hydrocarbon ring compounds known in the art as ones satisfying the above number of carbon atoms.

The present disclosure provides a heterocyclic compound represented by Chemical Formula 1 below.

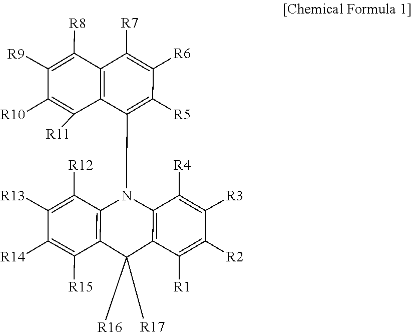

In Chemical Formula 1,
R1 to R17 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and a group represented by Chemical Formula 2 below, or two or more groups adjacent to each other are bonded to each other to form a substituted or unsubstituted C6 to C60 aromatic hydrocarbon ring or a substituted or unsubstituted C2 to C60 hetero ring, wherein R101, R102, and R103 are the same as or different from each other, and each independently a substituted or unsubstituted C1 to C60 alkyl group; a substituted or unsubstituted C6 to C60 aryl group; or a substituted or unsubstituted C2 to C60heteroaryl group,
at least one of R1 to R4 and R12 to R15 is a substituted or unsubstituted C6 to C60 aryl group; a substituted or unsubstituted C2 to C60heteroaryl group; or a group represented by Chemical Formula 2 below,
at least one of R5 to R11 is a substituted or unsubstituted C6 to C60 aryl group; a substituted or unsubstituted C2 to C60 heteroaryl group; or a group represented by Chemical Formula 2 below,

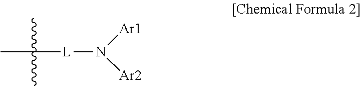

In Chemical Formula 2,
Ar1 and Ar2 are the same as or different from each other, and each independently a substituted or unsubstituted C6 to C60 aryl group; or a substituted or unsubstituted C2 to C60 heteroaryl group, and
L is a direct bond; a substituted or unsubstituted C6 to C60 arylene group; or a substituted or unsubstituted C2 to C60 heteroarylene group.

In an embodiment of the present disclosure, R1 to R15 may be the same as or different from each other, and each independently hydrogen; deuterium; halogen; a cyano group; a substituted or unsubstituted C1 to C30alkyl group; a substituted or unsubstituted C2 to C30alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30heteroaryl group; —P(═O)R101R102; —SiR101R102R103; or a group represented by Chemical Formula 2 above.

In another embodiment of the present disclosure, R1 to R15 may be the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; or a group represented by Chemical Formula 2 above.

In another embodiment of the present disclosure, R1 to R15 may be the same as or different from each other, and each independently hydrogen; deuterium; a substituted or unsubstituted C6 to C20 aryl group; a substituted or unsubstituted C2 to C20 heteroaryl group; or a group represented by Chemical Formula 2 above.

In another embodiment of the present disclosure, R1 to R15 may be the same as or different from each other, and each independently hydrogen; deuterium; a substituted or unsubstituted phenyl group; a substituted or unsubstituted naphthyl group; a substituted or unsubstituted fluorenyl group; a substituted or unsubstituted phenanthrenyl group; a substituted or unsubstituted isochrysenyl group; a substituted or unsubstituted spirobifluorenyl group; a substituted or unsubstituted dibenzofuranyl group; a substituted or unsubstituted dibenzothiophenyl group; or a group represented by Chemical Formula 2 above.

In an embodiment of the present disclosure, at least one of R1 to R4 and R12 to R15 may be a substituted or unsubstituted C6 to C30 aryl group; a substituted or unsubstituted C2 to C30 heteroaryl group; or a group represented by Chemical Formula 2 above.

In another embodiment of the present disclosure, at least one of R1 to R4 and R12 to R15 may be a substituted or unsubstituted C6 to C20 aryl group; a substituted or unsubstituted C2 to C20 heteroaryl group; or a group represented by Chemical Formula 2 above.

In another embodiment of the present disclosure, at least one of R1 to R4 and R12 to R15 may be a substituted or unsubstituted phenyl group; a substituted or unsubstituted naphthyl group; a substituted or unsubstituted fluorenyl group; a substituted or unsubstituted phenanthrenyl group; a substituted or unsubstituted isochrysenyl group; a substituted or unsubstituted spirobifluorenyl group; a substituted or unsubstituted dibenzofuranyl group; a substituted or unsubstituted dibenzothiophenyl group; or a group represented by Chemical Formula 2 above.

In an embodiment of the present disclosure, at least one of R5 to R11 may be a substituted or unsubstituted C6 to C30 aryl group; a substituted or unsubstituted C2 to C30 heteroaryl group; or a group represented by Chemical Formula 2 above.

In another embodiment of the present disclosure, at least one of R5 to R11 may be a substituted or unsubstituted C6 to C20 aryl group; a substituted or unsubstituted C2 to C20 heteroaryl group; or a group represented by Chemical Formula 2 above.

In another embodiment of the present disclosure, at least one of R5 to R11 may be a substituted or unsubstituted phenyl group; a substituted or unsubstituted naphthyl group; a substituted or unsubstituted fluorenyl group; a substituted or unsubstituted phenanthrenyl group; a substituted or unsubstituted isochrysenyl group; a substituted or unsubstituted spirobifluorenyl group; a substituted or unsubstituted dibenzofuranyl group; a substituted or unsubstituted dibenzothiophenyl group; or a group represented by Chemical Formula 2 above.

In an embodiment of the present disclosure, R5 may be a substituted or unsubstituted C6 to C60 aryl group; a substituted or unsubstituted C2 to C60 heteroaryl group; or a group represented by Chemical Formula 2 above, and at least one of R1 to R3 and R13 to R15 may be a substituted or unsubstituted C6 to C60 aryl group; a substituted or unsubstituted C2 to C60 heteroaryl group; or a group represented by Chemical Formula 2 above.

In another embodiment of the present disclosure, R5 may be substituted or unsubstituted C2 to C30 aryl group; a a substituted or unsubstituted C2 to C30 heteroaryl group; or a group represented by Chemical Formula 2 above, and at least one of R1 to R3 and R13 to R15 may be a substituted or unsubstituted C6 to C30 aryl group; a substituted or unsubstituted C2 to C30 heteroaryl group; or a group represented by Chemical Formula 2 above.

In another embodiment of the present disclosure, R5 may be a substituted or unsubstituted C6 to C20 aryl group; a substituted or unsubstituted C2 to C20 heteroaryl group; or a group represented by Chemical Formula 2 above, and at least one of R1 to R3 and R13 to R15 may be a substituted or unsubstituted C6 to C20 aryl group; a substituted or unsubstituted C2 to C20 heteroaryl group; or a group represented by Chemical Formula 2 above.

In another embodiment of the present disclosure, R5 may be a substituted or unsubstituted phenyl group; a substituted or unsubstituted naphthyl group; a substituted or unsubstituted fluorenyl group; a substituted or unsubstituted phenanthrenyl group; a substituted or unsubstituted isochrysenyl group; a substituted or unsubstituted spirobifluorenyl group; a substituted or unsubstituted dibenzofuranyl group; a substituted or unsubstituted dibenzothiophenyl group; or a group represented by Chemical Formula 2 above, and at least one of R1 to R3 and R13 to R15 may be a substituted or unsubstituted phenyl group; a substituted or unsubstituted naphthyl group; a substituted or unsubstituted fluorenyl group; a substituted or unsubstituted phenanthrenyl group; a substituted or unsubstituted isochrysenyl group; a substituted or unsubstituted spirobifluorenyl group; a substituted or unsubstituted dibenzofuranyl group; a substituted or unsubstituted dibenzothiophenyl group; or a group represented by Chemical Formula 2 above.

In an embodiment of the present disclosure, when R5 is a substituted or unsubstituted C6 to C60 aryl group; or a substituted or unsubstituted C2 to C60 heteroaryl group, at least one of R1 to R3 and R13 to R15 may be a group represented by Chemical Formula 2 above.

In another embodiment of the present disclosure, when R5 is a substituted or unsubstituted C6 to C30 aryl group; or a substituted or unsubstituted C2 to C30 heteroaryl group, at least one of R1 to R3 and R13 to R15 may be a group represented by Chemical Formula 2 above.

In another embodiment of the present disclosure, when R5 is a substituted or unsubstituted C6 to C20 aryl group; or a substituted or unsubstituted C2 to C20 heteroaryl group, at least one of R1 to R3 and R13 to R15 may be a group represented by Chemical Formula 2 above.

In another embodiment of the present disclosure, when R5 is a substituted or unsubstituted phenyl group; a substituted or unsubstituted naphthyl group; a substituted or unsubstituted fluorenyl group; a substituted or unsubstituted phenanthrenyl group; a substituted or unsubstituted isochrysenyl group; a substituted or unsubstituted spirobifluorenyl group; a substituted or unsubstituted dibenzofuranyl group; or a substituted or unsubstituted dibenzothiophenyl group, at least one of R1 to R3 and R13 to R15 may be a group represented by Chemical Formula 2 above.

In another embodiment of the present disclosure, when R5 is a group represented by Chemical Formula 2 above, at least one of R1 to R3 and R13 to R15 may be a substituted or unsubstituted C6 to C60 aryl group; or a substituted or unsubstituted C2 to C60 heteroaryl group.

In another embodiment of the present disclosure, when R5 is a group represented by Chemical Formula 2 above, at least one of R1 to R3 and R13 to R15 may be a substituted or unsubstituted C6 to C30 aryl group; or a substituted or unsubstituted C2 to C30 heteroaryl group.

In another embodiment of the present disclosure, when R5 is a group represented by Chemical Formula 2 above, at least one of R1 to R3 and R13 to R15 may be a substituted or unsubstituted C6 to C20 aryl group; or a substituted or unsubstituted C2 to C20 heteroaryl group.

In another embodiment of the present disclosure, when R5 is a group represented by Chemical Formula 2 above, at least one of R1 to R3 and R13 to R15 may be a substituted or unsubstituted phenyl group; a substituted or unsubstituted naphthyl group; a substituted or unsubstituted fluorenyl group; a substituted or unsubstituted phenanthrenyl group; a substituted or unsubstituted isochrysenyl group; a substituted or unsubstituted spirobifluorenyl group; a substituted or unsubstituted dibenzofuranyl group; or a substituted or unsubstituted dibenzothiophenyl group.

In an embodiment of the present disclosure, R16 and R17 may be the same as or different from each other, and each independently a substituted or unsubstituted C1 to C20 alkyl group.

In another embodiment of the present disclosure, R16 and R17 may be the same as or different from each other, and each independently a substituted or unsubstituted C1 to C10 alkyl group.

In another embodiment of the present disclosure, R16 and R17 may be the same as or different from each other, and each independently a substituted or unsubstituted methyl group; a substituted or unsubstituted ethyl group; or a substituted or unsubstituted propyl group.

In another embodiment of the present disclosure, both R16 and R17 may be a methyl group.

In an embodiment of the present disclosure, the content of deuterium may be 0% or more, 10% or more, 20% or more, 30% or more, 40% or more, or 50% or more, and may be 100% or less, 90% or less, 80% or less, 70% or less, or 60% or less based on the total number of hydrogen atoms and deuterium atoms in Chemical Formula 1 above.

In another embodiment of the present disclosure, the content of deuterium may be 30% to 100% based on the total number of hydrogen atoms and deuterium atoms in Chemical Formula 1 above.

In another embodiment of the present disclosure, the content of deuterium may be 30% to 80% based on the total number of hydrogen atoms and deuterium atoms in Chemical Formula 1 above.

In another embodiment of the present disclosure, the content of deuterium may be 50% to 60% based on the total number of hydrogen atoms and deuterium atoms in Chemical Formula 1 above.

In an embodiment of the present disclosure, Ar1 and Ar2 may be the same as or different from each other, and each independently a substituted or unsubstituted C6 to C30 aryl group; or a substituted or unsubstituted C2 to C30 heteroaryl group.

In another embodiment of the present disclosure, Ar1 and Ar2 may be the same as or different from each other, and each independently a substituted or unsubstituted C6 to C20 aryl group; or a substituted or unsubstituted C2 to C20 heteroaryl group.

In another embodiment of the present disclosure, Ar1 and Ar2 may be the same as or different from each other, and each independently a substituted or unsubstituted phenyl group; a substituted or unsubstituted naphthyl group; a substituted or unsubstituted fluorenyl group; a substituted or unsubstituted phenanthrenyl group; a substituted or unsubstituted isochrysenyl group; a substituted or unsubstituted spirobifluorenyl group; a substituted or unsubstituted dibenzofuranyl group; or a substituted or unsubstituted dibenzothiophenyl group.

In an embodiment of the present disclosure, L may be a direct bond; a substituted or unsubstituted C6 to C30 arylene group; or a substituted or unsubstituted C2 to C30 heteroarylene group.

In another embodiment of the present disclosure, L may be a direct bond; a substituted or unsubstituted C6 to C20 arylene group; or a substituted or unsubstituted C2 to C20 heteroarylene group.

In another embodiment of the present disclosure, L may be a direct bond; a substituted or unsubstituted phenylene group; or a substituted or unsubstituted biphenylene group.

Specific examples of L are shown below, but L is not limited to these examples.

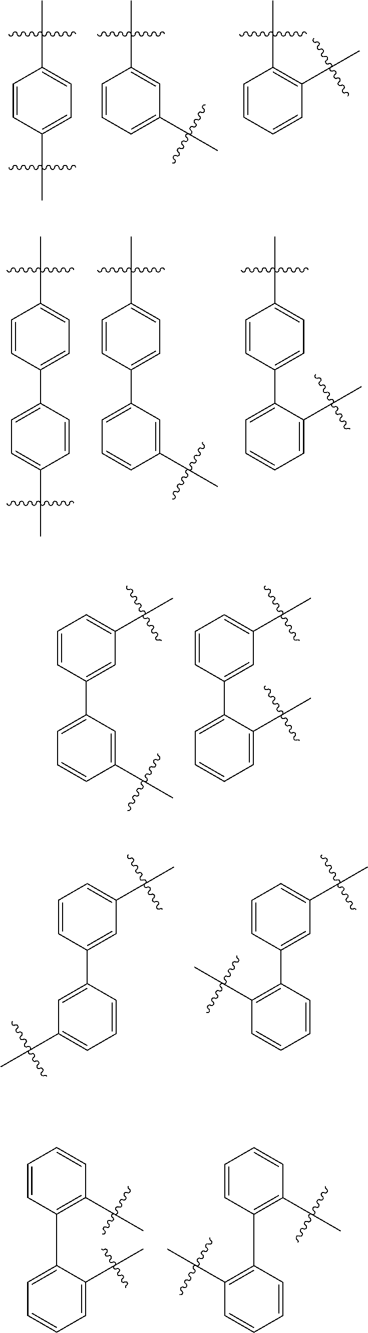

In an embodiment of the present disclosure, Chemical Formula 1 above may be a heterocyclic compound represented by any one of the compounds below.

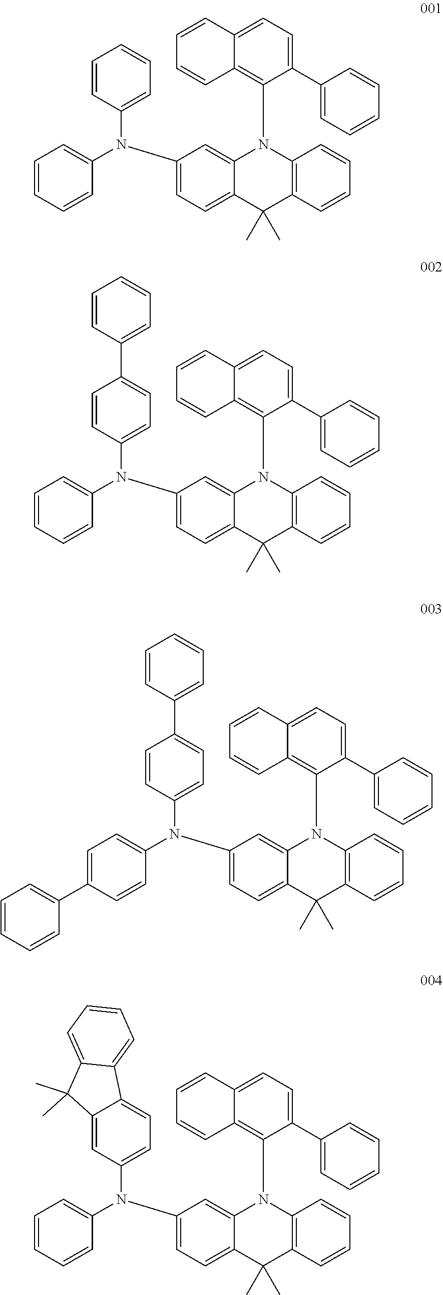 [SMILES: CC1(C)c2ccccc2N(c2c(-c3ccccc3)ccc3ccccc23)c2cc(N(c3ccccc3)c3ccccc3)ccc21 ; CC1(C)c2ccccc2N(c2c(-c3ccccc3)ccc3ccccc23)c2cc(N(c3ccccc3)c3ccc(-c4ccccc4)cc3)ccc21 ; CC1(C)c2ccccc2N(c2c(-c3ccccc3)ccc3ccccc23)c2cc(N(c3ccc(-c4ccccc4)cc3)c3ccc(-c4ccccc4)cc3)ccc21 ; CC1(C)c2ccccc2-c2ccc(N(c3ccccc3)c3ccc4c(c3)N(c3c(-c5ccccc5)ccc5ccccc35)c3ccccc3C4(C)C)cc21]

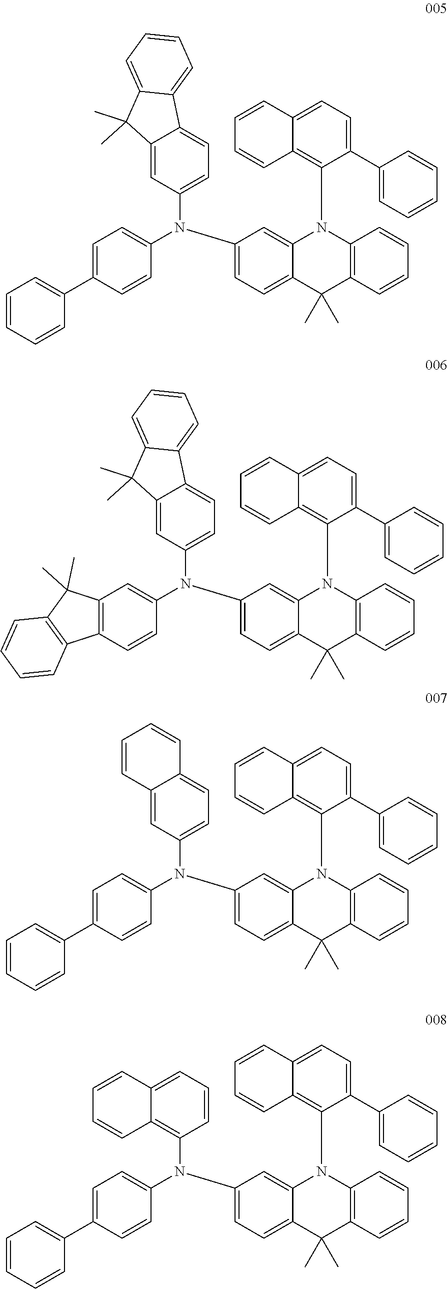 [SMILES: CC1(C)c2ccccc2-c2ccc(N(c3ccc(-c4ccccc4)cc3)c3ccc4c(c3)N(c3c(-c5ccccc5)ccc5ccccc35)c3ccccc3C4(C)C)cc21 ; CC1(C)c2ccccc2-c2ccc(N(c3ccc4c(c3)N(c3c(-c5ccccc5)ccc5ccccc35)c3ccccc3C4(C)C)c3ccc4c(c3)C(C)(C)c3ccccc3-4)cc21 ; CC1(C)c2ccccc2N(c2c(-c3ccccc3)ccc3ccccc23)c2cc(N(c3ccc(-c4ccccc4)cc3)c3ccc4ccccc4c3)ccc21 ; CC1(C)c2ccccc2N(c2c(-c3ccccc3)ccc3ccccc23)c2cc(N(c3ccc(-c4ccccc4)cc3)c3cccc4ccccc34)ccc21]

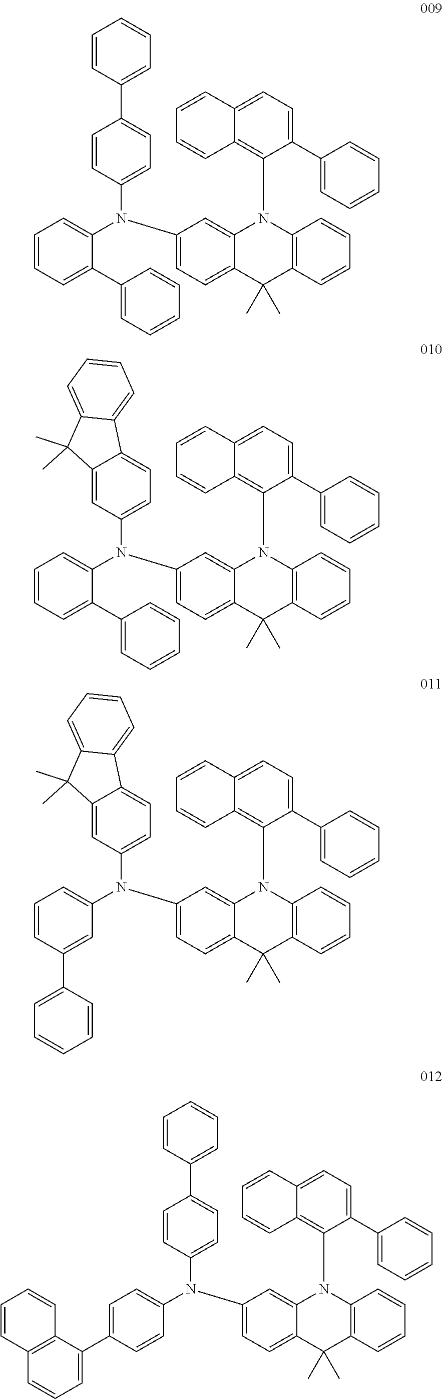

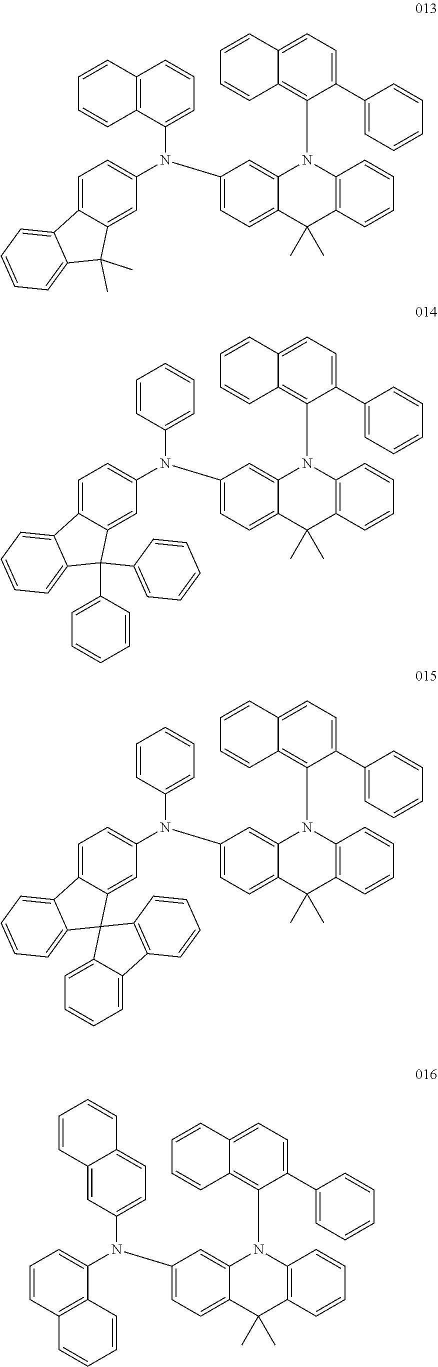 [SMILES: CC1(C)c2ccccc2-c2ccc(N(c3ccc4c(c3)N(c3c(-c5ccccc5)ccc5ccccc35)c3ccccc3C4(C)C)c3cccc4ccccc34)cc21 ; CC1(C)c2ccccc2N(c2c(-c3ccccc3)ccc3ccccc23)c2cc(N(c3ccccc3)c3ccc4c(c3)C(c3ccccc3)(c3ccccc3)c3ccccc3-4)ccc21 ; CC1(C)c2ccccc2N(c2c(-c3ccccc3)ccc3ccccc23)c2cc(N(c3ccccc3)c3ccc4c(c3)C3(c5ccccc5-c5ccccc53)c3ccccc3-4)ccc21 ; CC1(C)c2ccccc2N(c2c(-c3ccccc3)ccc3ccccc23)c2cc(N(c3ccc4ccccc4c3)c3cccc4ccccc34)ccc21]

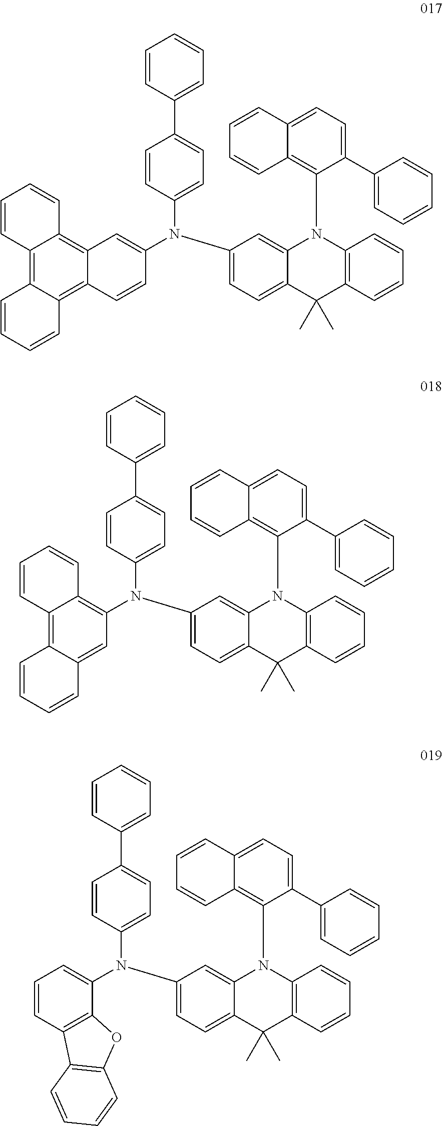 [SMILES: CC1(C)c2ccccc2N(c2c(-c3ccccc3)ccc3ccccc23)c2cc(N(c3ccc(-c4ccccc4)cc3)c3ccc4c5ccccc5c5ccccc5c4c3)ccc21 ; CC1(C)c2ccccc2N(c2c(-c3ccccc3)ccc3ccccc23)c2cc(N(c3ccc(-c4ccccc4)cc3)c3cc4ccccc4c4ccccc34)ccc21 ; CC1(C)c2ccccc2N(c2c(-c3ccccc3)ccc3ccccc23)c2cc(N(c3ccc(-c4ccccc4)cc3)c3cccc4c3oc3ccccc34)ccc21]

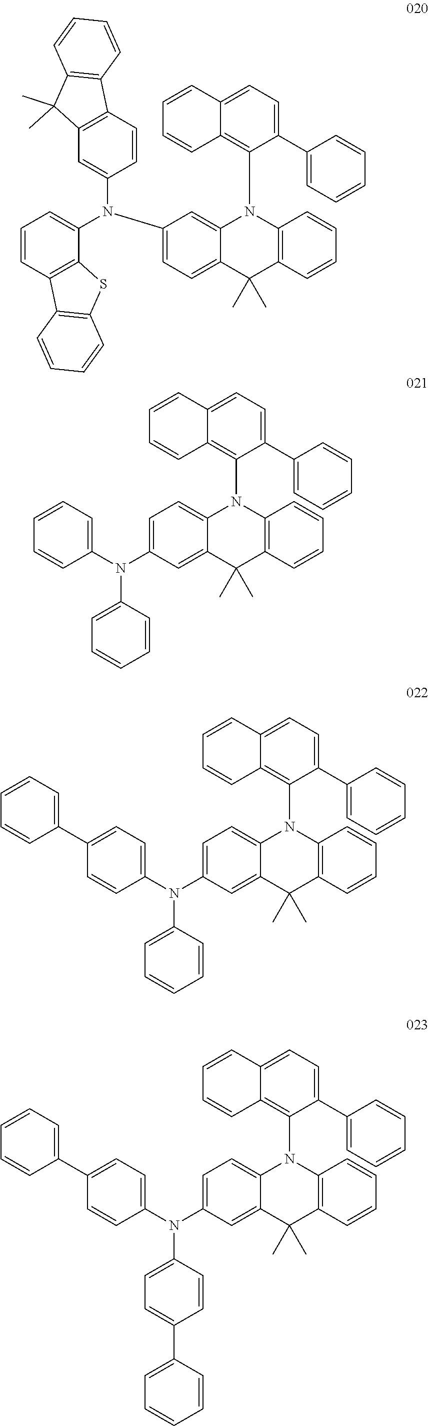 [SMILES: CC1(C)c2ccccc2-c2ccc(N(c3ccc4c(c3)N(c3c(-c5ccccc5)ccc5ccccc35)c3ccccc3C4(C)C)c3cccc4c3sc3ccccc34)cc21 ; CC1(C)c2ccccc2N(c2c(-c3ccccc3)ccc3ccccc23)c2ccc(N(c3ccccc3)c3ccccc3)cc21 ; CC1(C)c2ccccc2N(c2c(-c3ccccc3)ccc3ccccc23)c2ccc(N(c3ccccc3)c3ccc(-c4ccccc4)cc3)cc21 ; CC1(C)c2ccccc2N(c2c(-c3ccccc3)ccc3ccccc23)c2ccc(N(c3ccc(-c4ccccc4)cc3)c3ccc(-c4ccccc4)cc3)cc21]

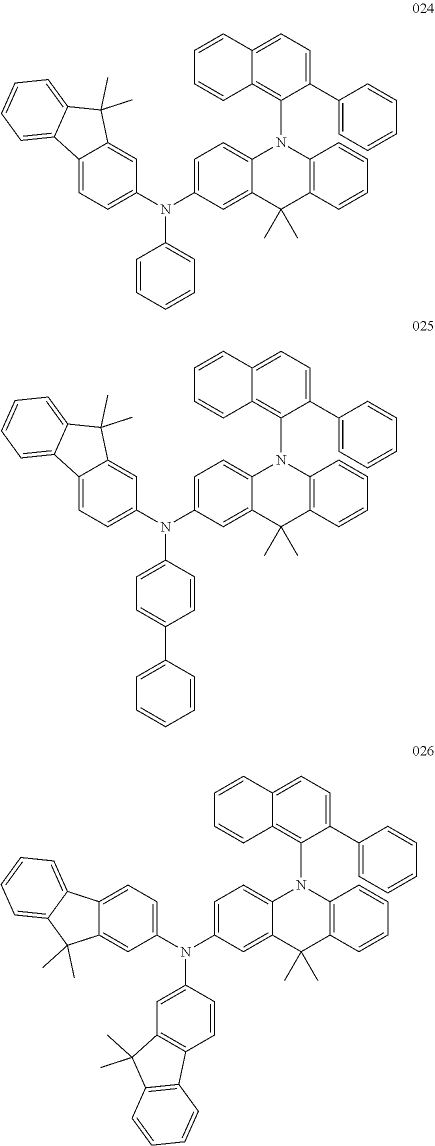 [SMILES: CC1(C)c2ccccc2-c2ccc(N(c3ccccc3)c3ccc4c(c3)C(C)(C)c3ccccc3N4c3c(-c4ccccc4)ccc4ccccc34)cc21 ; CC1(C)c2ccccc2-c2ccc(N(c3ccc(-c4ccccc4)cc3)c3ccc4c(c3)C(C)(C)c3ccccc3N4c3c(-c4ccccc4)ccc4ccccc34)cc21 ; CC1(C)c2ccccc2-c2ccc(N(c3ccc4c(c3)C(C)(C)c3ccccc3-4)c3ccc4c(c3)C(C)(C)c3ccccc3N4c3c(-c4ccccc4)ccc4ccccc34)cc21]

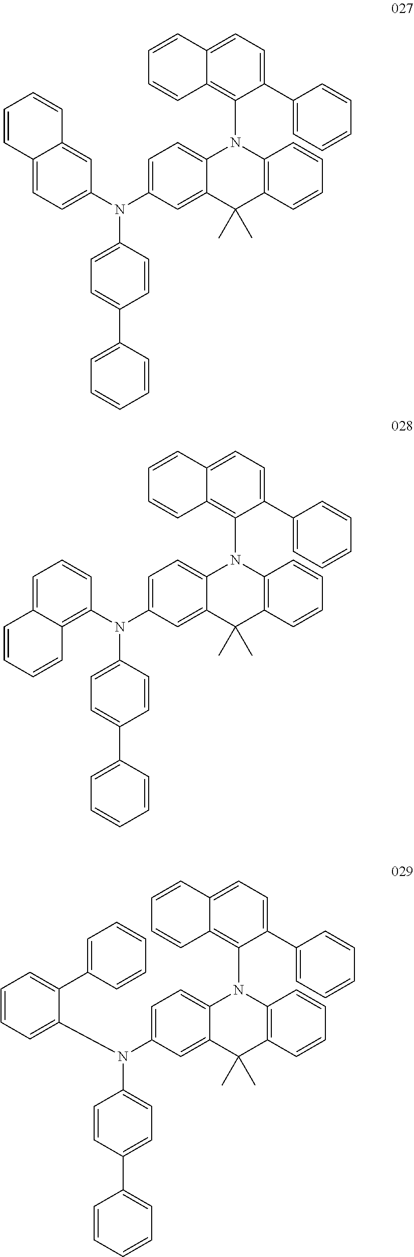 [SMILES: CC1(C)c2ccccc2N(c2c(-c3ccccc3)ccc3ccccc23)c2ccc(N(c3ccc(-c4ccccc4)cc3)c3ccc4ccccc4c3)cc21 ; CC1(C)c2ccccc2N(c2c(-c3ccccc3)ccc3ccccc23)c2ccc(N(c3ccc(-c4ccccc4)cc3)c3cccc4ccccc34)cc21 ; CC1(C)c2ccccc2N(c2c(-c3ccccc3)ccc3ccccc23)c2ccc(N(c3ccc(-c4ccccc4)cc3)c3ccccc3-c3ccccc3)cc21]

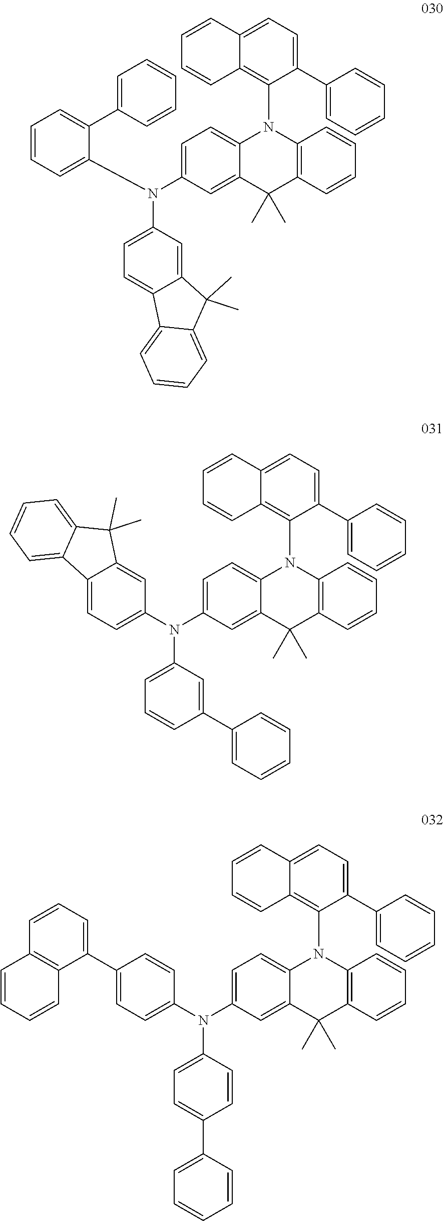 [SMILES: CC1(C)c2ccccc2-c2ccc(N(c3ccc4c(c3)C(C)(C)c3ccccc3N4c3c(-c4ccccc4)ccc4ccccc34)c3ccccc3-c3ccccc3)cc21 ; CC1(C)c2ccccc2-c2ccc(N(c3cccc(-c4ccccc4)c3)c3ccc4c(c3)C(C)(C)c3ccccc3N4c3c(-c4ccccc4)ccc4ccccc34)cc21 ; CC1(C)c2ccccc2N(c2c(-c3ccccc3)ccc3ccccc23)c2ccc(N(c3ccc(-c4ccccc4)cc3)c3ccc(-c4cccc5ccccc45)cc3)cc21]

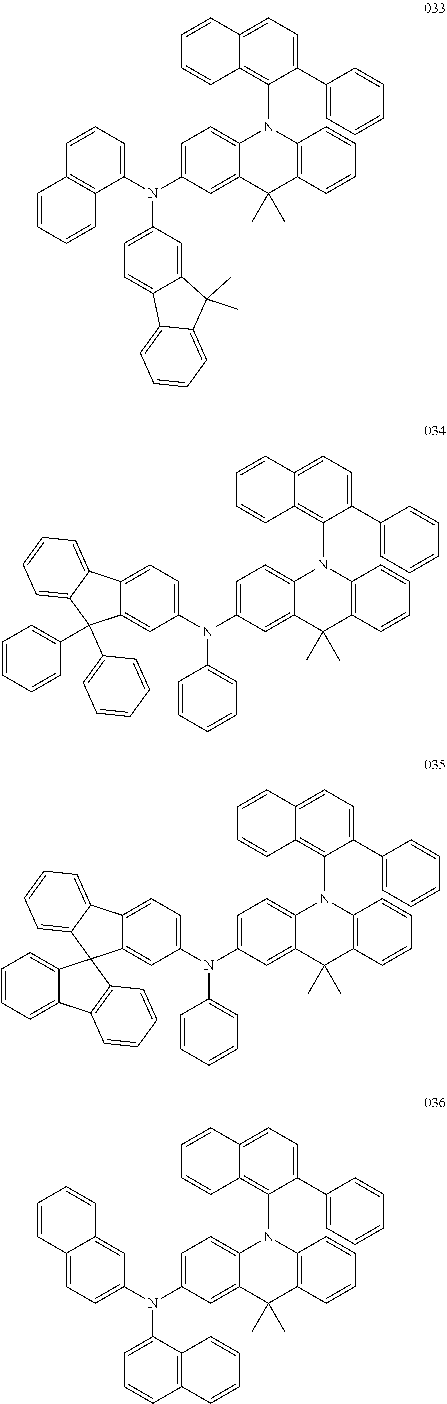 [SMILES: CC1(C)c2ccccc2-c2ccc(N(c3ccc4c(c3)C(C)(C)c3ccccc3N4c3c(-c4ccccc4)ccc4ccccc34)c3cccc4ccccc34)cc21 ; CC1(C)c2ccccc2N(c2c(-c3ccccc3)ccc3ccccc23)c2ccc(N(c3ccccc3)c3ccc4c(c3)C(c3ccccc3)(c3ccccc3)c3ccccc3-4)cc21 ; CC1(C)c2ccccc2N(c2c(-c3ccccc3)ccc3ccccc23)c2ccc(N(c3ccccc3)c3ccc4c(c3)C3(c5ccccc5-c5ccccc53)c3ccccc3-4)cc21 ; CC1(C)c2ccccc2N(c2c(-c3ccccc3)ccc3ccccc23)c2ccc(N(c3ccc4ccccc4c3)c3cccc4ccccc34)cc21]

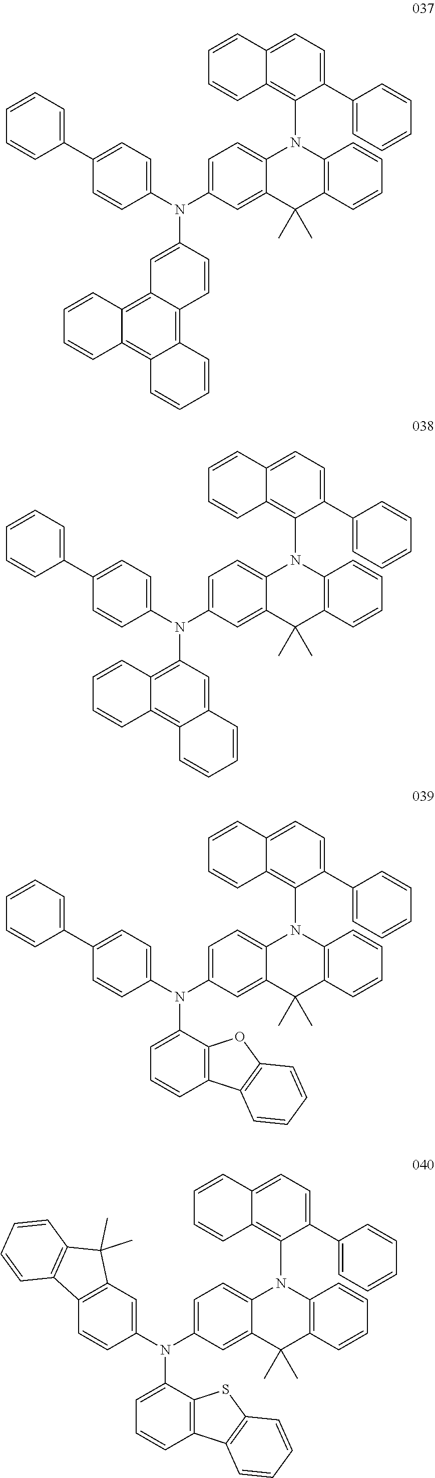 [SMILES: CC1(C)c2ccccc2N(c2c(-c3ccccc3)ccc3ccccc23)c2ccc(N(c3ccc(-c4ccccc4)cc3)c3ccc4c5ccccc5c5ccccc5c4c3)cc21 ; CC1(C)c2ccccc2N(c2c(-c3ccccc3)ccc3ccccc23)c2ccc(N(c3ccc(-c4ccccc4)cc3)c3cc4ccccc4c4ccccc34)cc21 ; CC1(C)c2ccccc2N(c2c(-c3ccccc3)ccc3ccccc23)c2ccc(N(c3ccc(-c4ccccc4)cc3)c3cccc4c3oc3ccccc34)cc21 ; CC1(C)c2ccccc2-c2ccc(N(c3ccc4c(c3)C(C)(C)c3ccccc3N4c3c(-c4ccccc4)ccc4ccccc34)c3cccc4c3sc3ccccc34)cc21]

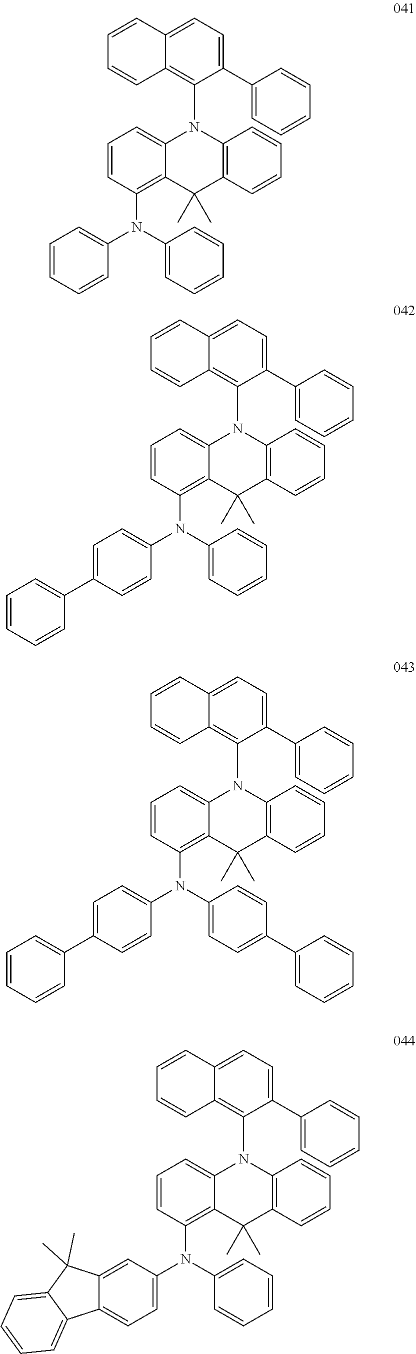

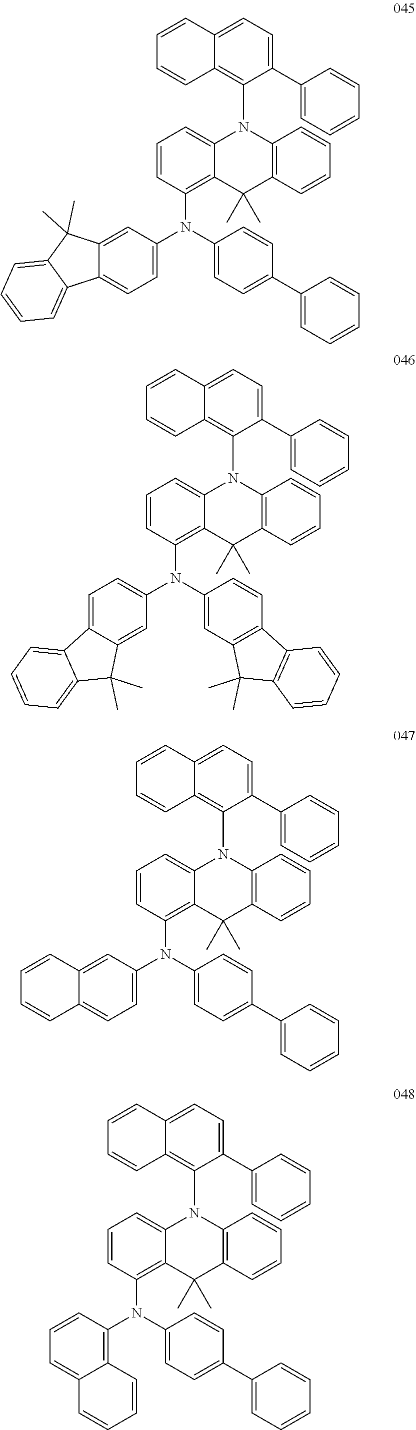

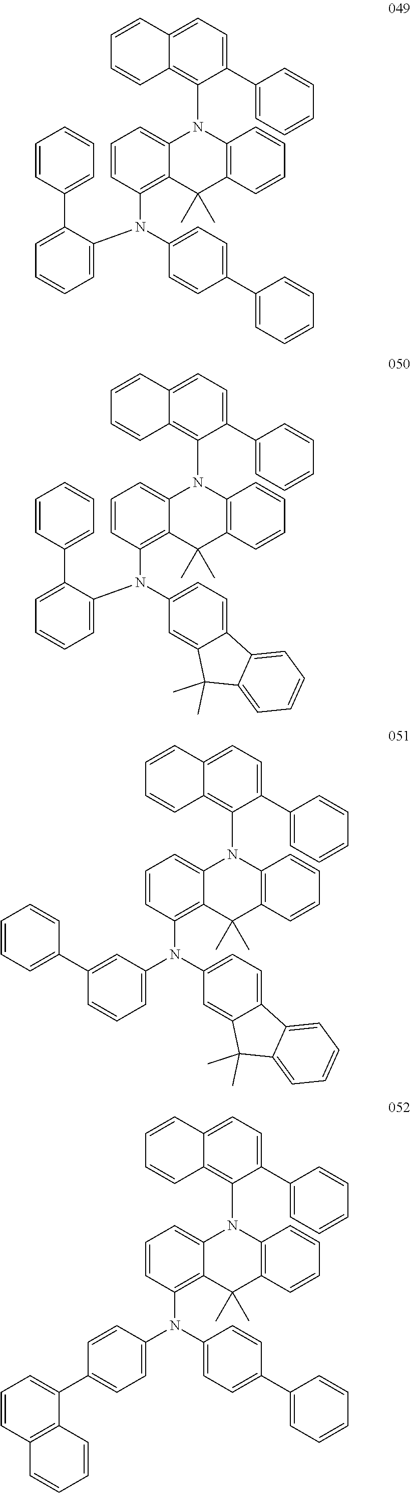

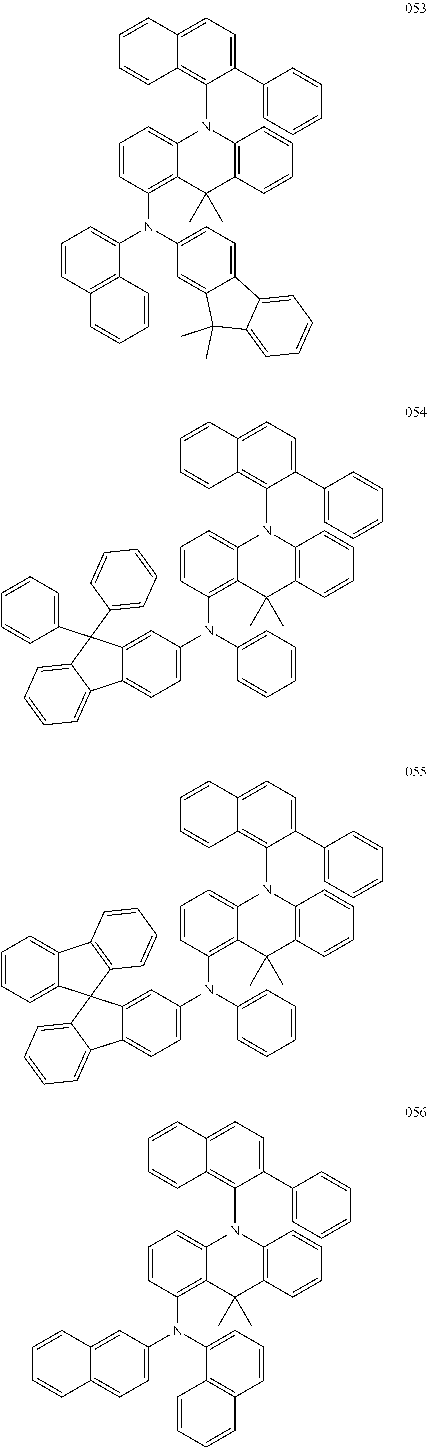 [SMILES: CC1(C)c2ccccc2-c2ccc(N(c3cccc4c3C(C)(C)c3ccccc3N4c3c(-c4ccccc4)ccc4ccccc34)c3cccc4ccccc34)cc21 ; CC1(C)c2ccccc2N(c2c(-c3ccccc3)ccc3ccccc23)c2cccc(N(c3ccccc3)c3ccc4c(c3)C(c3ccccc3)(c3ccccc3)c3ccccc3-4)c21 ; CC1(C)c2ccccc2N(c2c(-c3ccccc3)ccc3ccccc23)c2cccc(N(c3ccccc3)c3ccc4c(c3)C3(c5ccccc5-c5ccccc53)c3ccccc3-4)c21 ; CC1(C)c2ccccc2N(c2c(-c3ccccc3)ccc3ccccc23)c2cccc(N(c3ccc4ccccc4c3)c3cccc4ccccc34)c21]

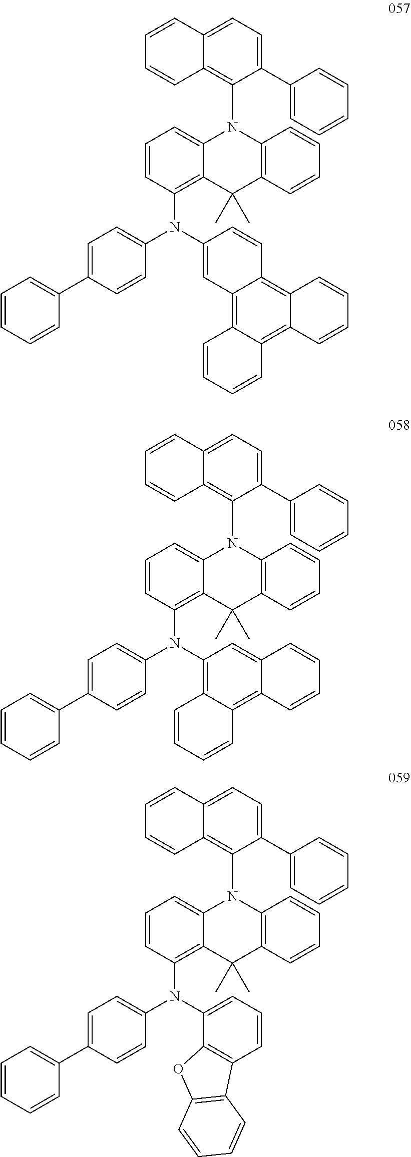

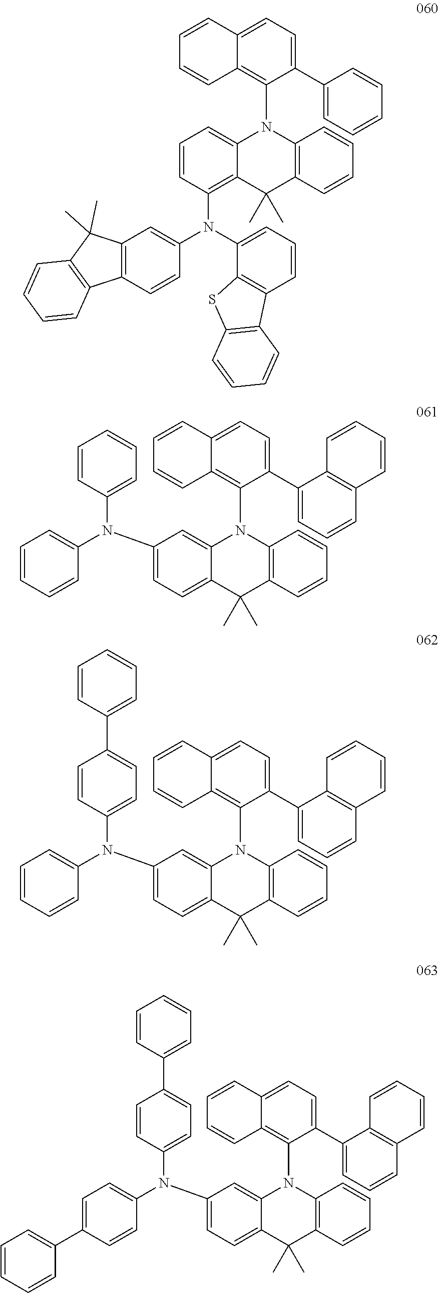

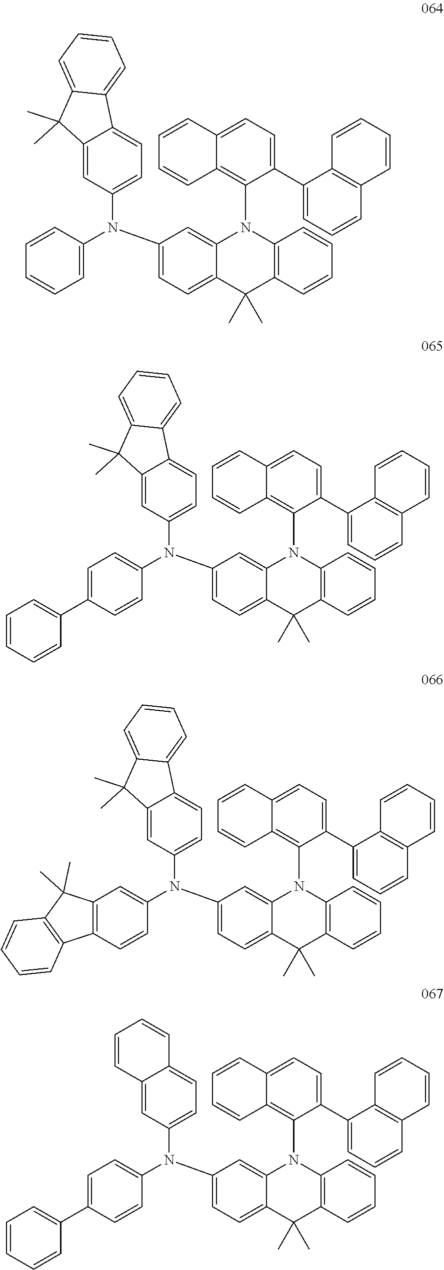 [SMILES: CC1(C)c2ccccc2-c2ccc(N(c3ccccc3)c3ccc4c(c3)N(c3c(-c5cccc6ccccc56)ccc5ccccc35)c3ccccc3C4(C)C)cc21 ; CC1(C)c2ccccc2-c2ccc(N(c3ccc(-c4ccccc4)cc3)c3ccc4c(c3)N(c3c(-c5cccc6ccccc56)ccc5ccccc35)c3ccccc3C4(C)C)cc21 ; CC1(C)c2ccccc2-c2ccc(N(c3ccc4c(c3)N(c3c(-c5cccc6ccccc56)ccc5ccccc35)c3ccccc3C4(C)C)c3ccc4c(c3)C(C)(C)c3ccccc3-4)cc21 ; CC1(C)c2ccccc2N(c2c(-c3cccc4ccccc34)ccc3ccccc23)c2cc(N(c3ccc(-c4ccccc4)cc3)c3ccc4ccccc4c3)ccc21]

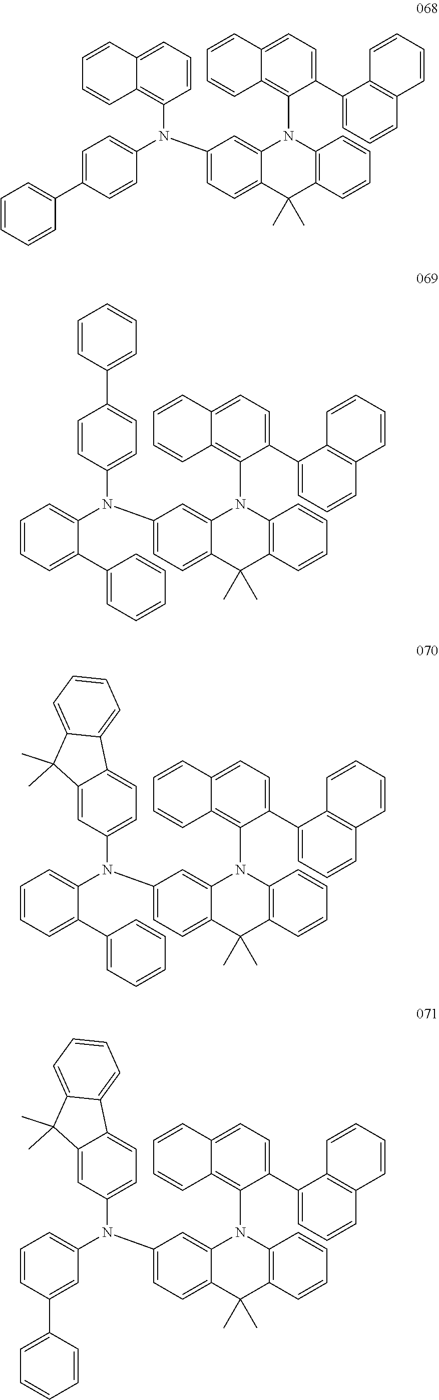 [SMILES: CC1(C)c2ccccc2N(c2c(-c3cccc4ccccc34)ccc3ccccc23)c2cc(N(c3ccc(-c4ccccc4)cc3)c3cccc4ccccc34)ccc21 ; CC1(C)c2ccccc2N(c2c(-c3cccc4ccccc34)ccc3ccccc23)c2cc(N(c3ccc(-c4ccccc4)cc3)c3ccccc3-c3ccccc3)ccc21 ; CC1(C)c2ccccc2-c2ccc(N(c3ccc4c(c3)N(c3c(-c5cccc6ccccc56)ccc5ccccc35)c3ccccc3C4(C)C)c3ccccc3-c3ccccc3)cc21 ; CC1(C)c2ccccc2-c2ccc(N(c3cccc(-c4ccccc4)c3)c3ccc4c(c3)N(c3c(-c5cccc6ccccc56)ccc5ccccc35)c3ccccc3C4(C)C)cc21]

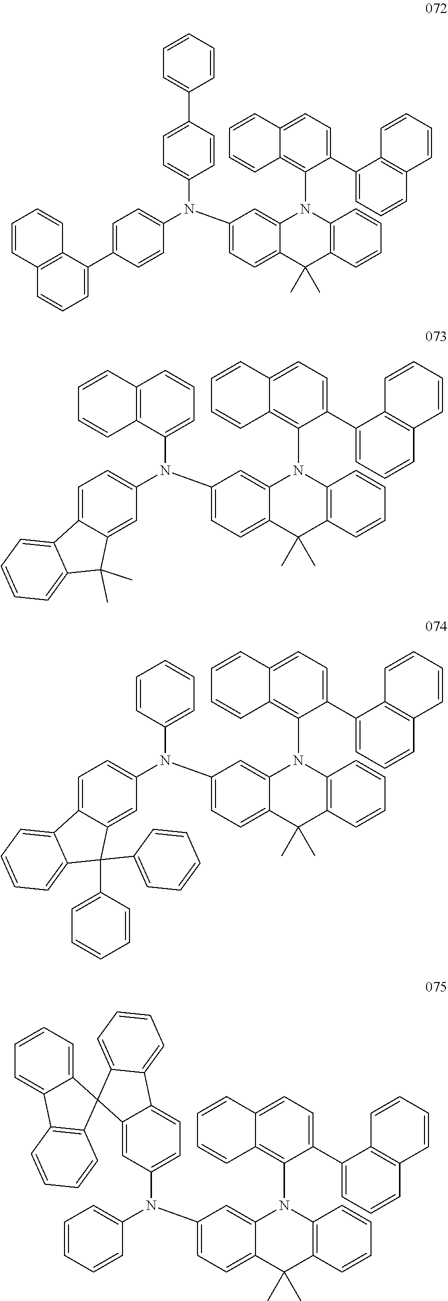 [SMILES: CC1(C)c2ccccc2N(c2c(-c3cccc4ccccc34)ccc3ccccc23)c2cc(N(c3ccc(-c4ccccc4)cc3)c3ccc(-c4cccc5ccccc45)cc3)ccc21 ; CC1(C)c2ccccc2-c2ccc(N(c3ccc4c(c3)N(c3c(-c5cccc6ccccc56)ccc5ccccc35)c3ccccc3C4(C)C)c3cccc4ccccc34)cc21 ; CC1(C)c2ccccc2N(c2c(-c3cccc4ccccc34)ccc3ccccc23)c2cc(N(c3ccccc3)c3ccc4c(c3)C(c3ccccc3)(c3ccccc3)c3ccccc3-4)ccc21 ; CC1(C)c2ccccc2N(c2c(-c3cccc4ccccc34)ccc3ccccc23)c2cc(N(c3ccccc3)c3ccc4c(c3)C3(c5ccccc5-c5ccccc53)c3ccccc3-4)ccc21]

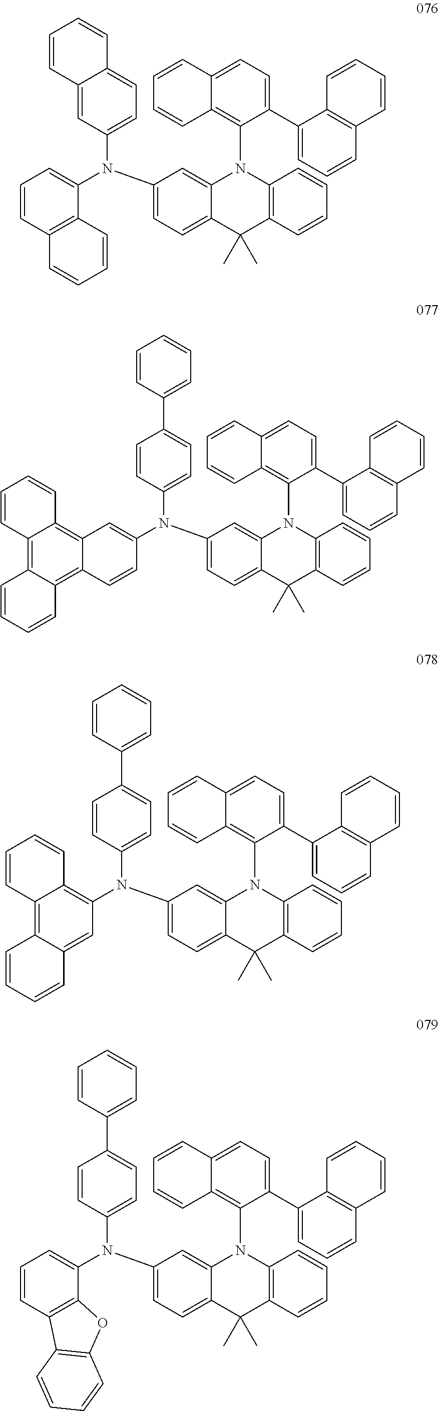 [SMILES: CC1(C)c2ccccc2N(c2c(-c3cccc4ccccc34)ccc3ccccc23)c2cc(N(c3ccc4ccccc4c3)c3cccc4ccccc34)ccc21 ; CC1(C)c2ccccc2N(c2c(-c3cccc4ccccc34)ccc3ccccc23)c2cc(N(c3ccc(-c4ccccc4)cc3)c3ccc4c5ccccc5c5ccccc5c4c3)ccc21 ; CC1(C)c2ccccc2N(c2c(-c3cccc4ccccc34)ccc3ccccc23)c2cc(N(c3ccc(-c4ccccc4)cc3)c3cc4ccccc4c4ccccc34)ccc21 ; CC1(C)c2ccccc2N(c2c(-c3cccc4ccccc34)ccc3ccccc23)c2cc(N(c3ccc(-c4ccccc4)cc3)c3cccc4c3oc3ccccc34)ccc21]

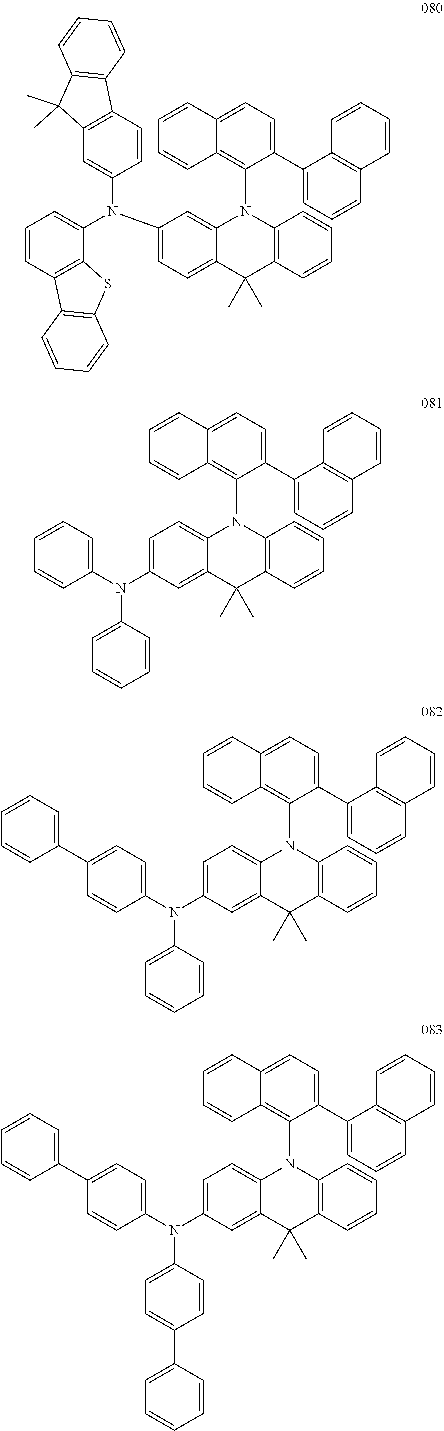 [SMILES: CC1(C)c2ccccc2-c2ccc(N(c3ccc4c(c3)N(c3c(-c5cccc6ccccc56)ccc5ccccc35)c3ccccc3C4(C)C)c3cccc4c3sc3ccccc34)cc21 ; CC1(C)c2ccccc2N(c2c(-c3cccc4ccccc34)ccc3ccccc23)c2ccc(N(c3ccccc3)c3ccccc3)cc21 ; CC1(C)c2ccccc2N(c2c(-c3cccc4ccccc34)ccc3ccccc23)c2ccc(N(c3ccccc3)c3ccc(-c4ccccc4)cc3)cc21 ; CC1(C)c2ccccc2N(c2c(-c3cccc4ccccc34)ccc3ccccc23)c2ccc(N(c3ccc(-c4ccccc4)cc3)c3ccc(-c4ccccc4)cc3)cc21]

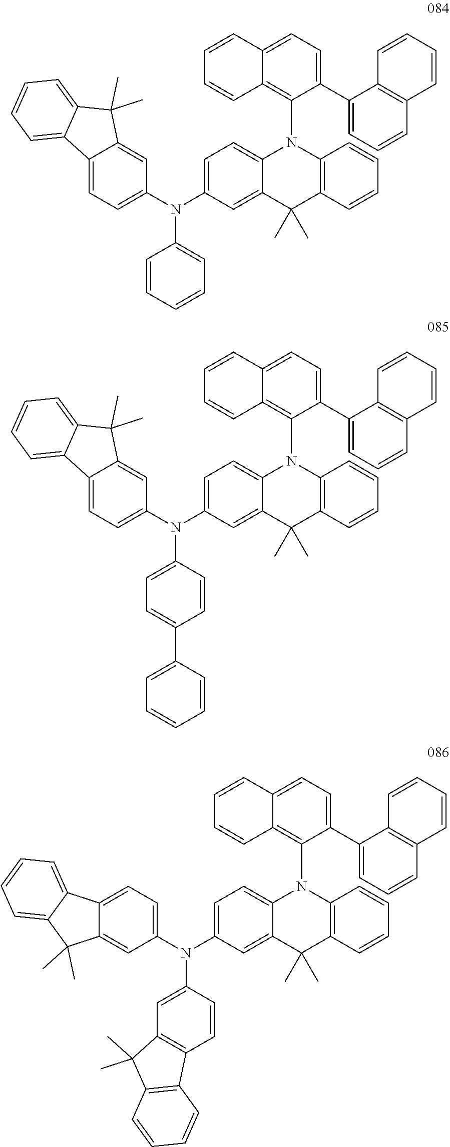 [SMILES: CC1(C)c2ccccc2-c2ccc(N(c3ccccc3)c3ccc4c(c3)C(C)(C)c3ccccc3N4c3c(-c4cccc5ccccc45)ccc4ccccc34)cc21 ; CC1(C)c2ccccc2-c2ccc(N(c3ccc(-c4ccccc4)cc3)c3ccc4c(c3)C(C)(C)c3ccccc3N4c3c(-c4cccc5ccccc45)ccc4ccccc34)cc21 ; CC1(C)c2ccccc2-c2ccc(N(c3ccc4c(c3)C(C)(C)c3ccccc3-4)c3ccc4c(c3)C(C)(C)c3ccccc3N4c3c(-c4cccc5ccccc45)ccc4ccccc34)cc21]

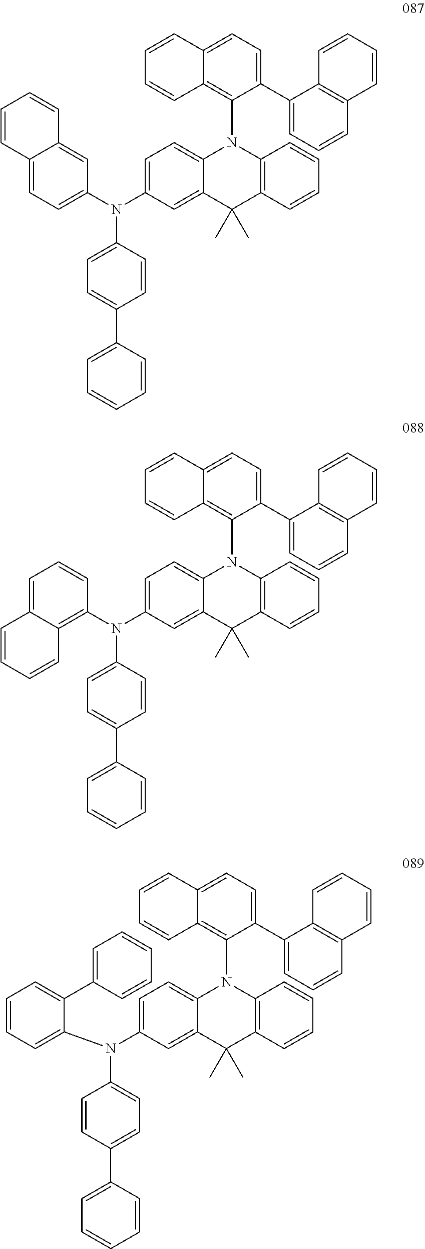 [SMILES: CC1(C)c2ccccc2N(c2c(-c3cccc4ccccc34)ccc3ccccc23)c2ccc(N(c3ccc(-c4ccccc4)cc3)c3ccc4ccccc4c3)cc21 ; CC1(C)c2ccccc2N(c2c(-c3cccc4ccccc34)ccc3ccccc23)c2ccc(N(c3ccc(-c4ccccc4)cc3)c3cccc4ccccc34)cc21 ; CC1(C)c2ccccc2N(c2c(-c3cccc4ccccc34)ccc3ccccc23)c2ccc(N(c3ccc(-c4ccccc4)cc3)c3ccccc3-c3ccccc3)cc21]

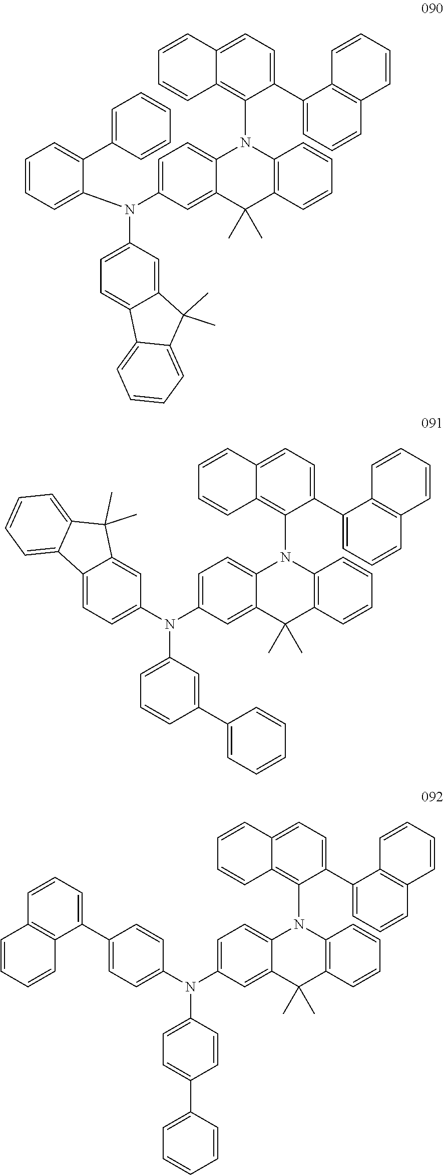 [SMILES: CC1(C)c2ccccc2-c2ccc(N(c3ccc4c(c3)C(C)(C)c3ccccc3N4c3c(-c4cccc5ccccc45)ccc4ccccc34)c3ccccc3-c3ccccc3)cc21 ; CC1(C)c2ccccc2-c2ccc(N(c3cccc(-c4ccccc4)c3)c3ccc4c(c3)C(C)(C)c3ccccc3N4c3c(-c4cccc5ccccc45)ccc4ccccc34)cc21 ; CC1(C)c2ccccc2N(c2c(-c3cccc4ccccc34)ccc3ccccc23)c2ccc(N(c3ccc(-c4ccccc4)cc3)c3ccc(-c4cccc5ccccc45)cc3)cc21]

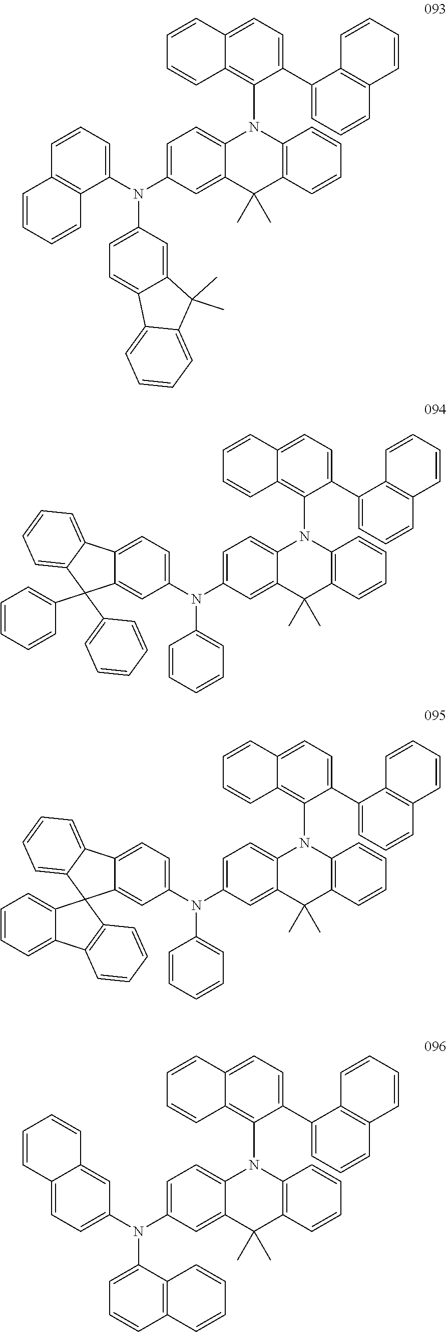 [SMILES: CC1(C)c2ccccc2-c2ccc(N(c3ccc4c(c3)C(C)(C)c3ccccc3N4c3c(-c4cccc5ccccc45)ccc4ccccc34)c3cccc4ccccc34)cc21 ; CC1(C)c2ccccc2N(c2c(-c3cccc4ccccc34)ccc3ccccc23)c2ccc(N(c3ccccc3)c3ccc4c(c3)C(c3ccccc3)(c3ccccc3)c3ccccc3-4)cc21 ; CC1(C)c2ccccc2N(c2c(-c3cccc4ccccc34)ccc3ccccc23)c2ccc(N(c3ccccc3)c3ccc4c(c3)C3(c5ccccc5-c5ccccc53)c3ccccc3-4)cc21 ; CC1(C)c2ccccc2N(c2c(-c3cccc4ccccc34)ccc3ccccc23)c2ccc(N(c3ccc4ccccc4c3)c3cccc4ccccc34)cc21]

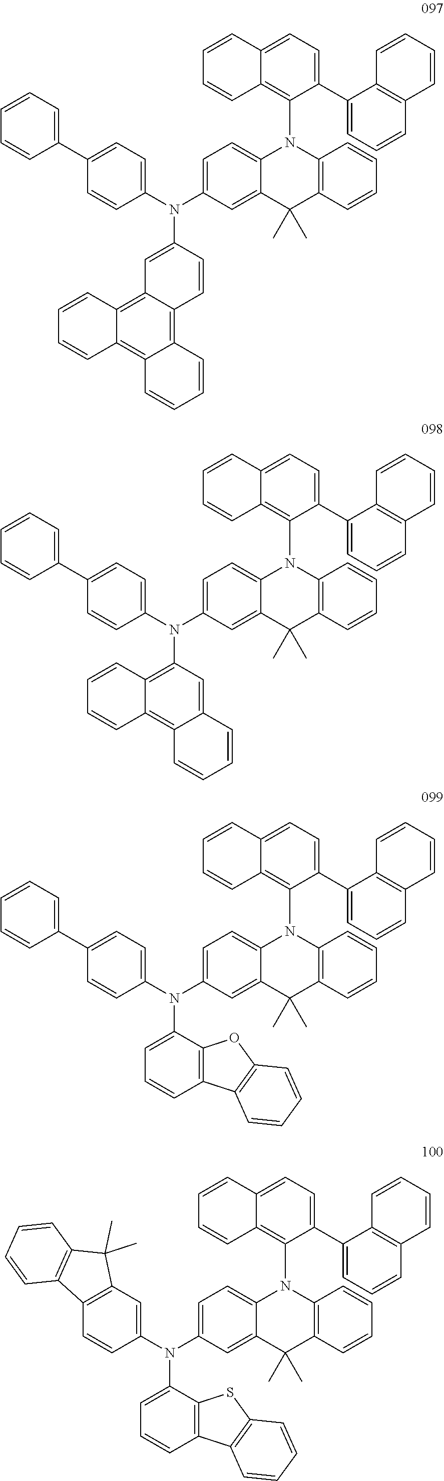 [SMILES: CC1(C)c2ccccc2N(c2c(-c3cccc4ccccc34)ccc3ccccc23)c2ccc(N(c3ccc(-c4ccccc4)cc3)c3ccc4c5ccccc5c5ccccc5c4c3)cc21 ; CC1(C)c2ccccc2N(c2c(-c3cccc4ccccc34)ccc3ccccc23)c2ccc(N(c3ccc(-c4ccccc4)cc3)c3cc4ccccc4c4ccccc34)cc21 ; CC1(C)c2ccccc2N(c2c(-c3cccc4ccccc34)ccc3ccccc23)c2ccc(N(c3ccc(-c4ccccc4)cc3)c3cccc4c3oc3ccccc34)cc21 ; CC1(C)c2ccccc2-c2ccc(N(c3ccc4c(c3)C(C)(C)c3ccccc3N4c3c(-c4cccc5ccccc45)ccc4ccccc34)c3cccc4c3sc3ccccc34)cc21]

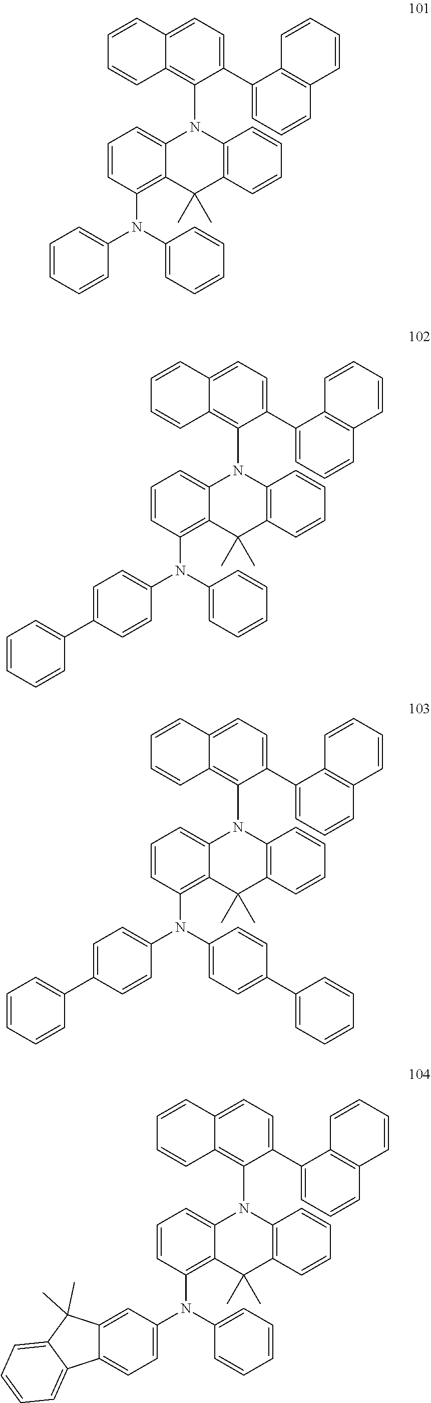 [SMILES: CC1(C)c2ccccc2N(c2c(-c3cccc4ccccc34)ccc3ccccc23)c2cccc(N(c3ccccc3)c3ccccc3)c21 ; CC1(C)c2ccccc2N(c2c(-c3cccc4ccccc34)ccc3ccccc23)c2cccc(N(c3ccccc3)c3ccc(-c4ccccc4)cc3)c21 ; CC1(C)c2ccccc2N(c2c(-c3cccc4ccccc34)ccc3ccccc23)c2cccc(N(c3ccc(-c4ccccc4)cc3)c3ccc(-c4ccccc4)cc3)c21 ; CC1(C)c2ccccc2-c2ccc(N(c3ccccc3)c3cccc4c3C(C)(C)c3ccccc3N4c3c(-c4cccc5ccccc45)ccc4ccccc34)cc21]

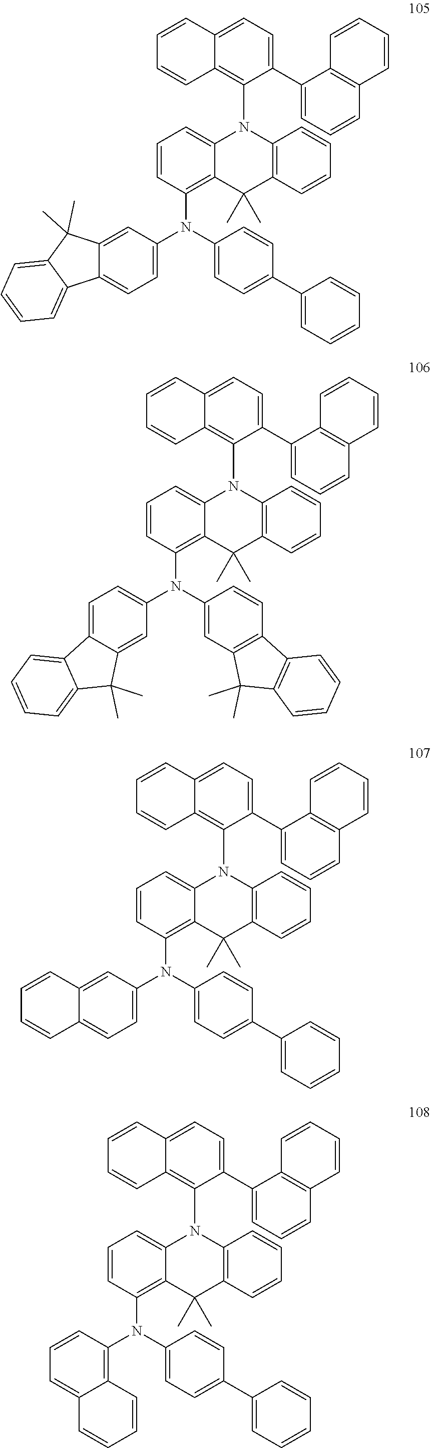

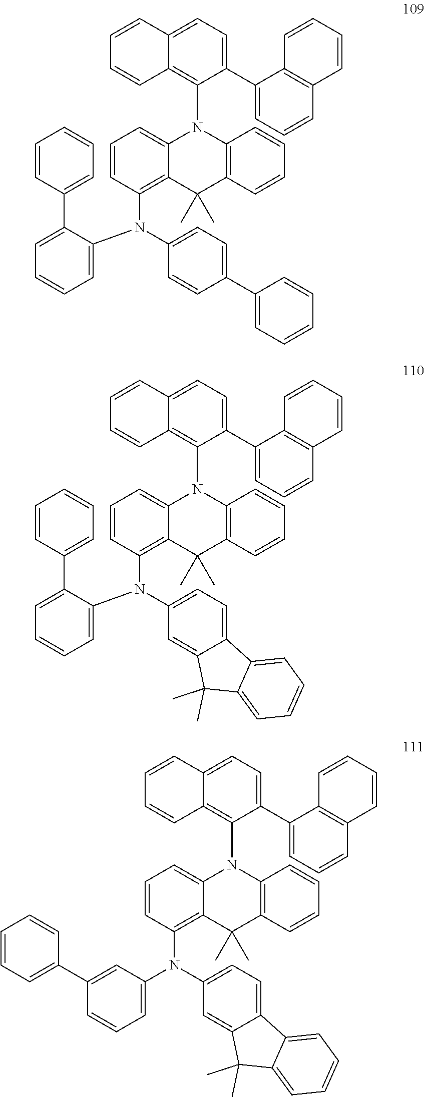

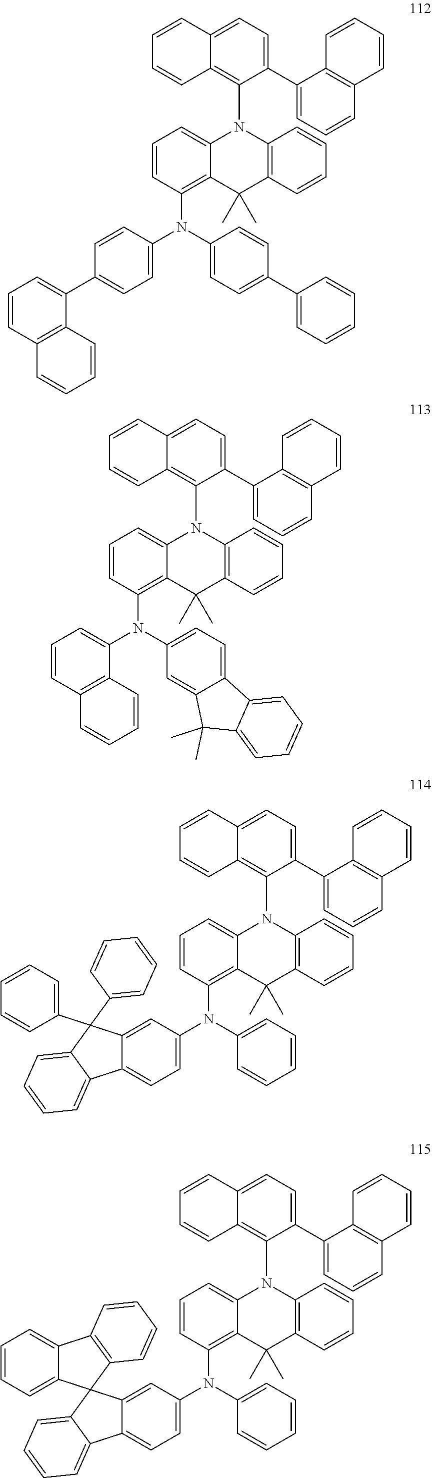 [SMILES: CC1(C)c2ccccc2N(c2c(-c3cccc4ccccc34)ccc3ccccc23)c2cccc(N(c3ccc(-c4ccccc4)cc3)c3ccc(-c4cccc5ccccc45)cc3)c21 ; CC1(C)c2ccccc2-c2ccc(N(c3cccc4c3C(C)(C)c3ccccc3N4c3c(-c4cccc5ccccc45)ccc4ccccc34)c3cccc4ccccc34)cc21 ; CC1(C)c2ccccc2N(c2c(-c3cccc4ccccc34)ccc3ccccc23)c2cccc(N(c3ccccc3)c3ccc4c(c3)C(c3ccccc3)(c3ccccc3)c3ccccc3-4)c21 ; CC1(C)c2ccccc2N(c2c(-c3cccc4ccccc34)ccc3ccccc23)c2cccc(N(c3ccccc3)c3ccc4c(c3)C3(c5ccccc5-c5ccccc53)c3ccccc3-4)c21]

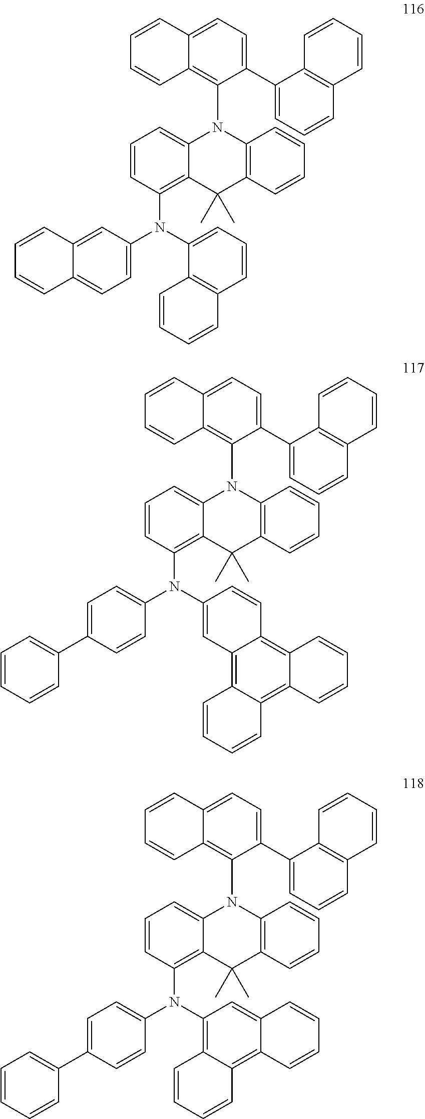

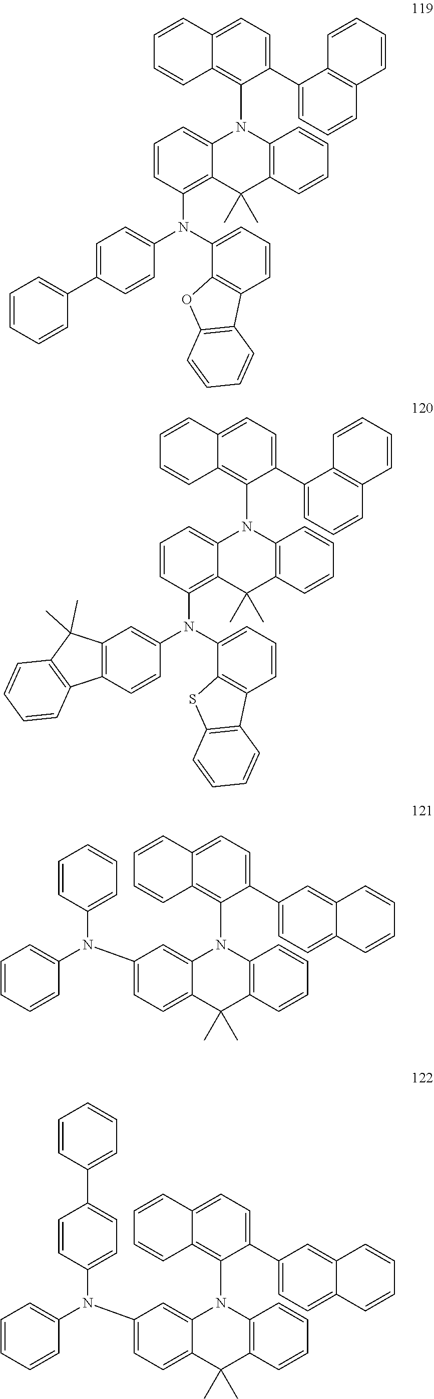

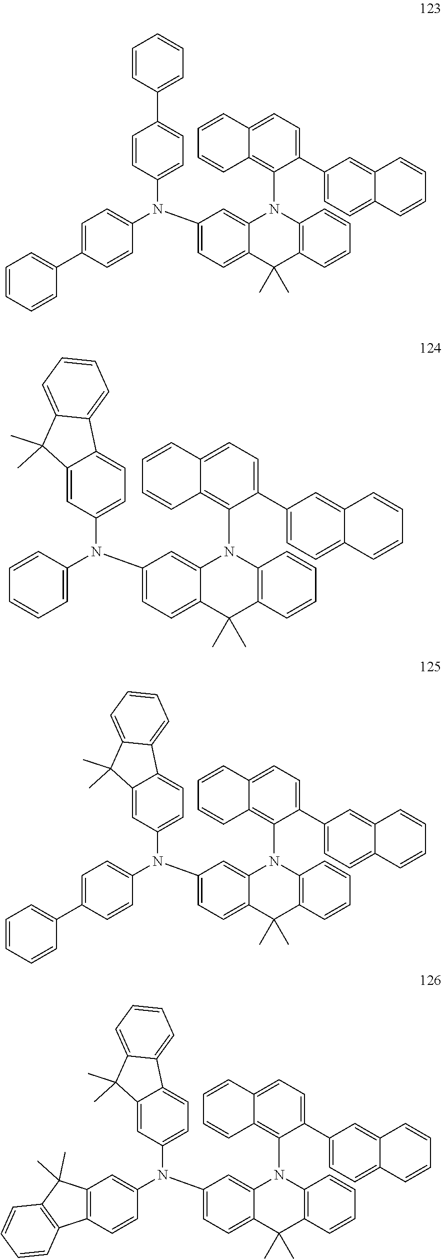 [SMILES: CC1(C)c2ccccc2N(c2c(-c3ccc4ccccc4c3)ccc3ccccc23)c2cc(N(c3ccc(-c4ccccc4)cc3)c3ccc(-c4ccccc4)cc3)ccc21 ; CC1(C)c2ccccc2-c2ccc(N(c3ccccc3)c3ccc4c(c3)N(c3c(-c5ccc6ccccc6c5)ccc5ccccc35)c3ccccc3C4(C)C)cc21 ; CC1(C)c2ccccc2-c2ccc(N(c3ccc(-c4ccccc4)cc3)c3ccc4c(c3)N(c3c(-c5ccc6ccccc6c5)ccc5ccccc35)c3ccccc3C4(C)C)cc21 ; CC1(C)c2ccccc2-c2ccc(N(c3ccc4c(c3)N(c3c(-c5ccc6ccccc6c5)ccc5ccccc35)c3ccccc3C4(C)C)c3ccc4c(c3)C(C)(C)c3ccccc3-4)cc21]

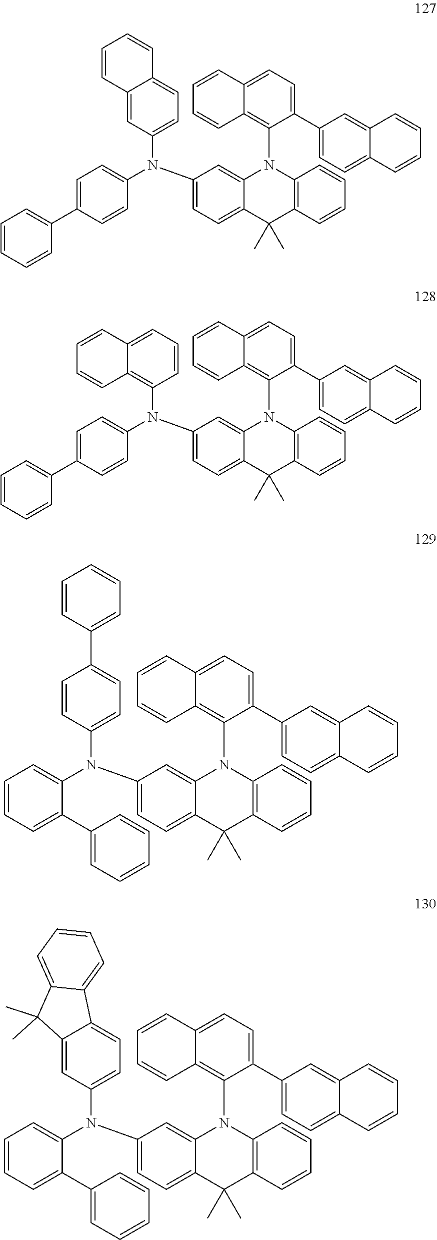 [SMILES: CC1(C)c2ccccc2N(c2c(-c3ccc4ccccc4c3)ccc3ccccc23)c2cc(N(c3ccc(-c4ccccc4)cc3)c3ccc4ccccc4c3)ccc21 ; CC1(C)c2ccccc2N(c2c(-c3ccc4ccccc4c3)ccc3ccccc23)c2cc(N(c3ccc(-c4ccccc4)cc3)c3cccc4ccccc34)ccc21 ; CC1(C)c2ccccc2N(c2c(-c3ccc4ccccc4c3)ccc3ccccc23)c2cc(N(c3ccc(-c4ccccc4)cc3)c3ccccc3-c3ccccc3)ccc21 ; CC1(C)c2ccccc2-c2ccc(N(c3ccc4c(c3)N(c3c(-c5ccc6ccccc6c5)ccc5ccccc35)c3ccccc3C4(C)C)c3ccccc3-c3ccccc3)cc21]

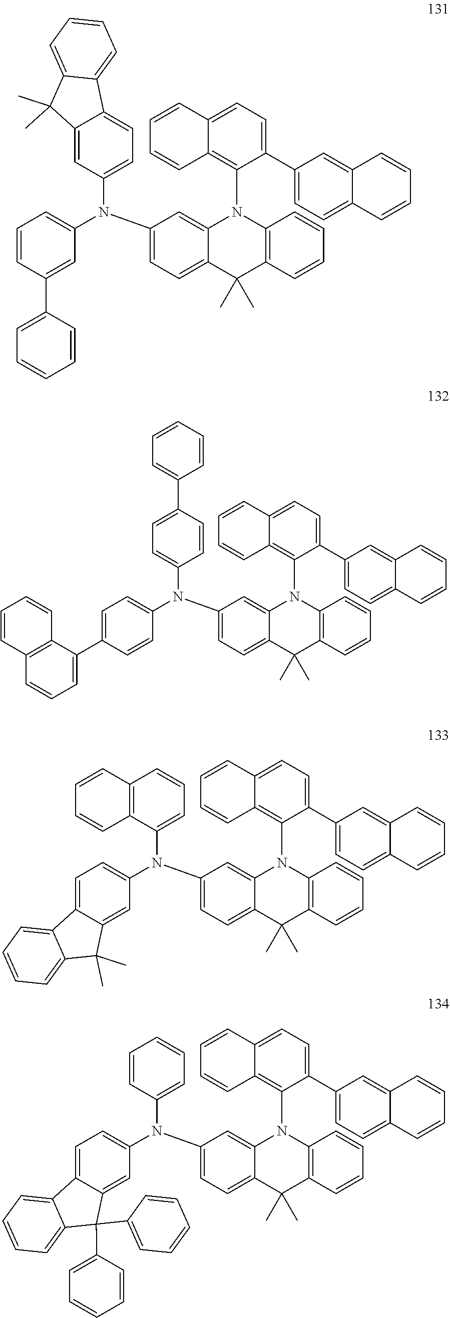 [SMILES: CC1(C)c2ccccc2-c2ccc(N(c3cccc(-c4ccccc4)c3)c3ccc4c(c3)N(c3c(-c5ccc6ccccc6c5)ccc5ccccc35)c3ccccc3C4(C)C)cc21 ; CC1(C)c2ccccc2N(c2c(-c3ccc4ccccc4c3)ccc3ccccc23)c2cc(N(c3ccc(-c4ccccc4)cc3)c3ccc(-c4cccc5ccccc45)cc3)ccc21 ; CC1(C)c2ccccc2-c2ccc(N(c3ccc4c(c3)N(c3c(-c5ccc6ccccc6c5)ccc5ccccc35)c3ccccc3C4(C)C)c3cccc4ccccc34)cc21 ; CC1(C)c2ccccc2N(c2c(-c3ccc4ccccc4c3)ccc3ccccc23)c2cc(N(c3ccccc3)c3ccc4c(c3)C(c3ccccc3)(c3ccccc3)c3ccccc3-4)ccc21]

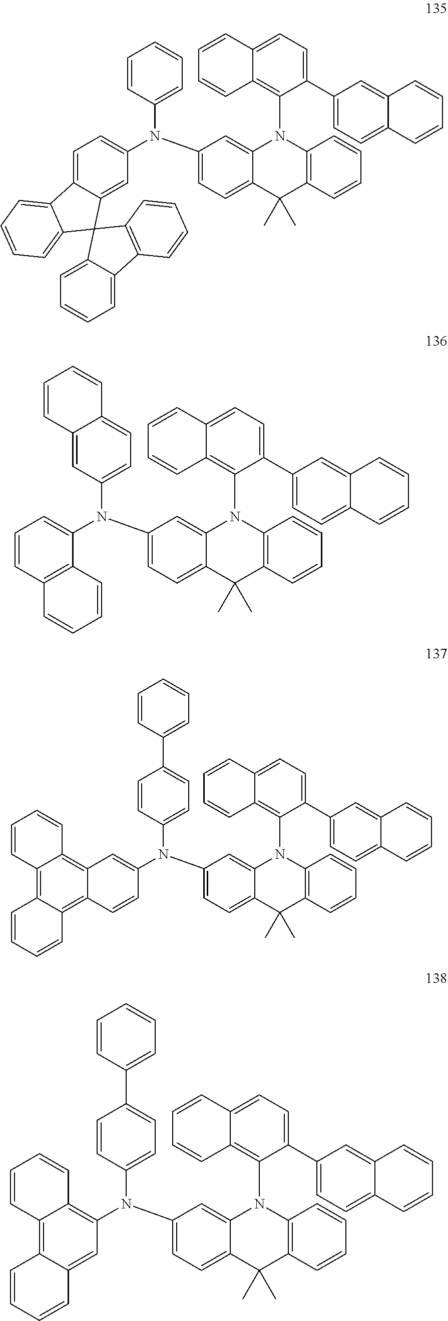 [SMILES: CC1(C)c2ccccc2N(c2c(-c3ccc4ccccc4c3)ccc3ccccc23)c2cc(N(c3ccccc3)c3ccc4c(c3)C3(c5ccccc5-c5ccccc53)c3ccccc3-4)ccc21 ; CC1(C)c2ccccc2N(c2c(-c3ccc4ccccc4c3)ccc3ccccc23)c2cc(N(c3ccc4ccccc4c3)c3cccc4ccccc34)ccc21 ; CC1(C)c2ccccc2N(c2c(-c3ccc4ccccc4c3)ccc3ccccc23)c2cc(N(c3ccc(-c4ccccc4)cc3)c3ccc4c5ccccc5c5ccccc5c4c3)ccc21 ; CC1(C)c2ccccc2N(c2c(-c3ccc4ccccc4c3)ccc3ccccc23)c2cc(N(c3ccc(-c4ccccc4)cc3)c3cc4ccccc4c4ccccc34)ccc21]

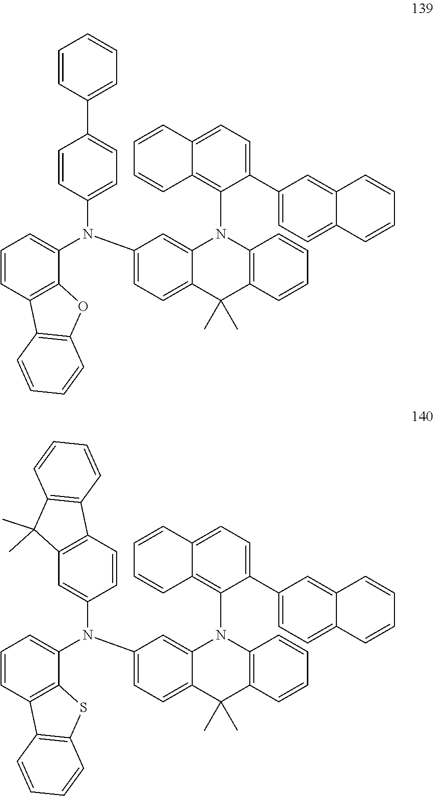 [SMILES: CC1(C)c2ccccc2N(c2c(-c3ccc4ccccc4c3)ccc3ccccc23)c2cc(N(c3ccc(-c4ccccc4)cc3)c3cccc4c3oc3ccccc34)ccc21 ; CC1(C)c2ccccc2-c2ccc(N(c3ccc4c(c3)N(c3c(-c5ccc6ccccc6c5)ccc5ccccc35)c3ccccc3C4(C)C)c3cccc4c3sc3ccccc34)cc21]

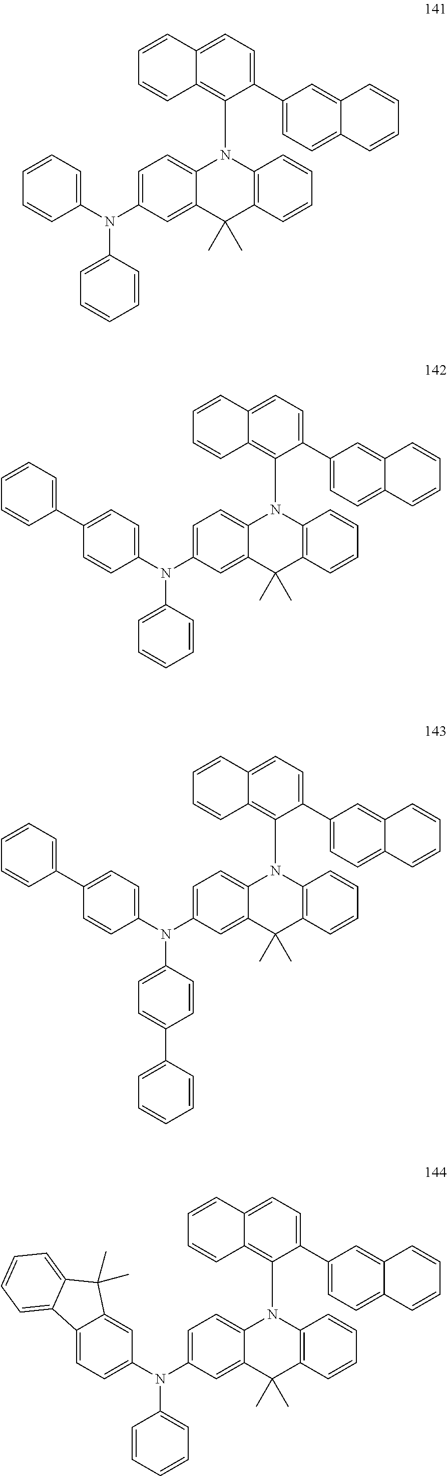 [SMILES: CC1(C)c2ccccc2N(c2c(-c3ccc4ccccc4c3)ccc3ccccc23)c2ccc(N(c3ccccc3)c3ccccc3)cc21 ; CC1(C)c2ccccc2N(c2c(-c3ccc4ccccc4c3)ccc3ccccc23)c2ccc(N(c3ccccc3)c3ccc(-c4ccccc4)cc3)cc21 ; CC1(C)c2ccccc2N(c2c(-c3ccc4ccccc4c3)ccc3ccccc23)c2ccc(N(c3ccc(-c4ccccc4)cc3)c3ccc(-c4ccccc4)cc3)cc21 ; CC1(C)c2ccccc2-c2ccc(N(c3ccccc3)c3ccc4c(c3)C(C)(C)c3ccccc3N4c3c(-c4ccc5ccccc5c4)ccc4ccccc34)cc21]

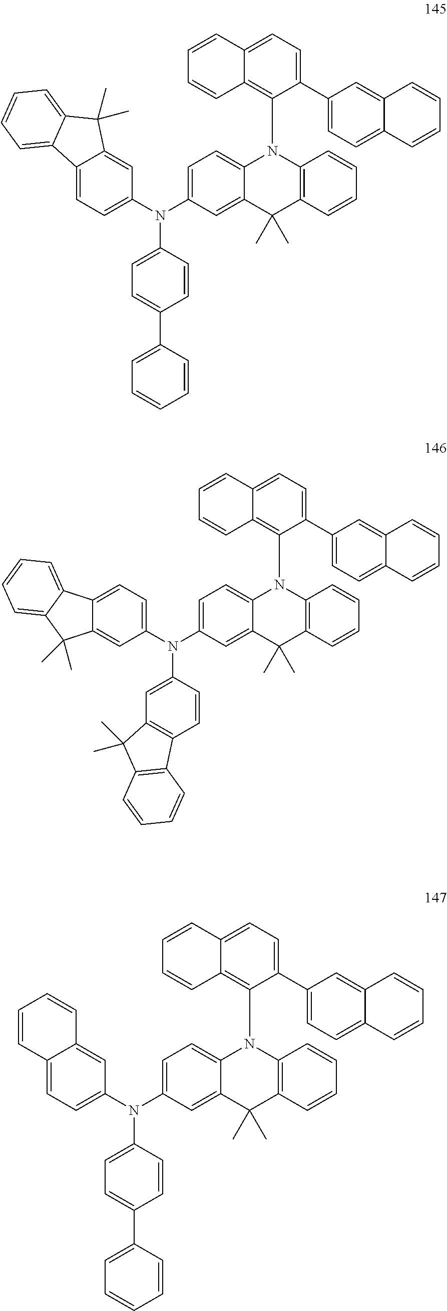 [SMILES: CC1(C)c2ccccc2-c2ccc(N(c3ccc(-c4ccccc4)cc3)c3ccc4c(c3)C(C)(C)c3ccccc3N4c3c(-c4ccc5ccccc5c4)ccc4ccccc34)cc21 ; CC1(C)c2ccccc2-c2ccc(N(c3ccc4c(c3)C(C)(C)c3ccccc3-4)c3ccc4c(c3)C(C)(C)c3ccccc3N4c3c(-c4ccc5ccccc5c4)ccc4ccccc34)cc21 ; CC1(C)c2ccccc2N(c2c(-c3ccc4ccccc4c3)ccc3ccccc23)c2ccc(N(c3ccc(-c4ccccc4)cc3)c3ccc4ccccc4c3)cc21]

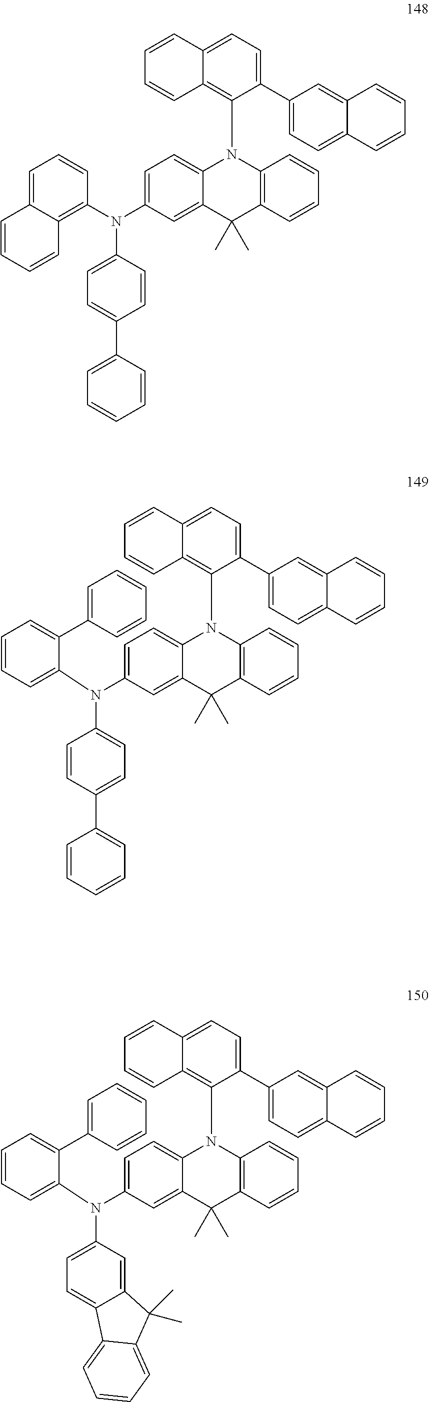 [SMILES: CC1(C)c2ccccc2N(c2c(-c3ccc4ccccc4c3)ccc3ccccc23)c2ccc(N(c3ccc(-c4ccccc4)cc3)c3cccc4ccccc34)cc21 ; CC1(C)c2ccccc2N(c2c(-c3ccc4ccccc4c3)ccc3ccccc23)c2ccc(N(c3ccc(-c4ccccc4)cc3)c3ccccc3-c3ccccc3)cc21 ; CC1(C)c2ccccc2-c2ccc(N(c3ccc4c(c3)C(C)(C)c3ccccc3N4c3c(-c4ccc5ccccc5c4)ccc4ccccc34)c3ccccc3-c3ccccc3)cc21]

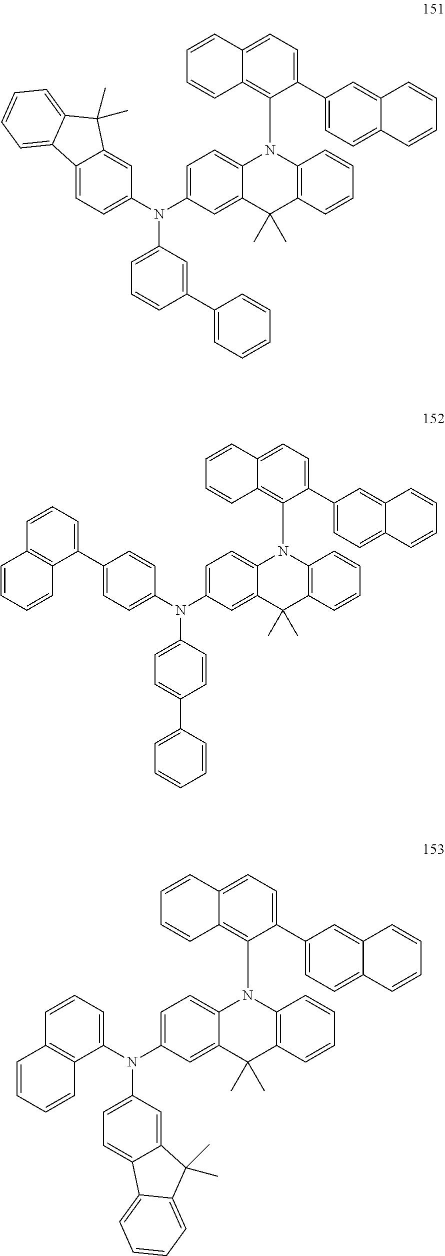 [SMILES: CC1(C)c2ccccc2-c2ccc(N(c3cccc(-c4ccccc4)c3)c3ccc4c(c3)C(C)(C)c3ccccc3N4c3c(-c4ccc5ccccc5c4)ccc4ccccc34)cc21 ; CC1(C)c2ccccc2N(c2c(-c3ccc4ccccc4c3)ccc3ccccc23)c2ccc(N(c3ccc(-c4ccccc4)cc3)c3ccc(-c4cccc5ccccc45)cc3)cc21 ; CC1(C)c2ccccc2-c2ccc(N(c3ccc4c(c3)C(C)(C)c3ccccc3N4c3c(-c4ccc5ccccc5c4)ccc4ccccc34)c3cccc4ccccc34)cc21]

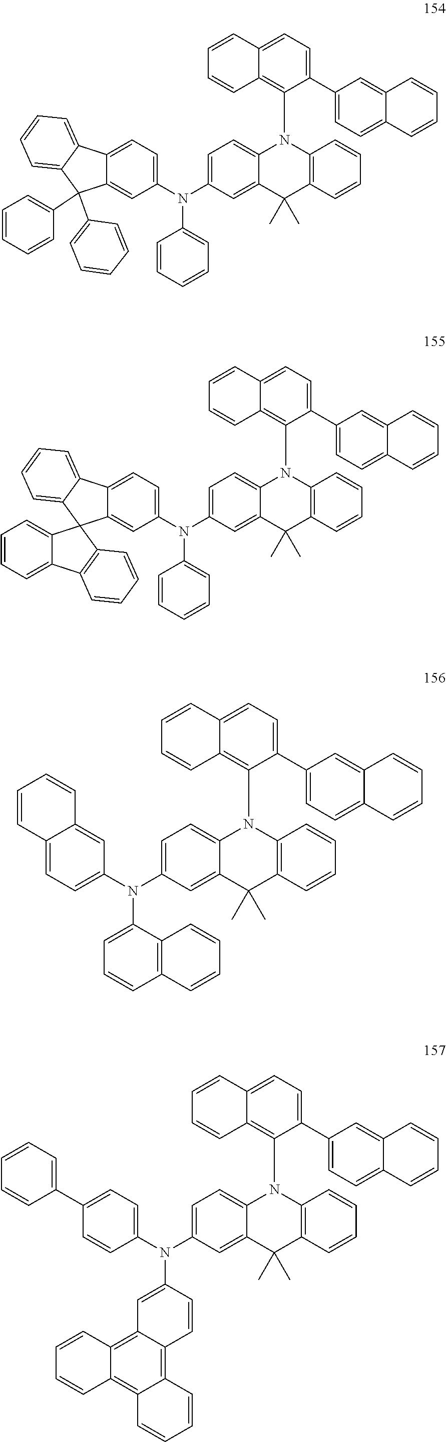 [SMILES: CC1(C)c2ccccc2N(c2c(-c3ccc4ccccc4c3)ccc3ccccc23)c2ccc(N(c3ccccc3)c3ccc4c(c3)C(c3ccccc3)(c3ccccc3)c3ccccc3-4)cc21 ; CC1(C)c2ccccc2N(c2c(-c3ccc4ccccc4c3)ccc3ccccc23)c2ccc(N(c3ccccc3)c3ccc4c(c3)C3(c5ccccc5-c5ccccc53)c3ccccc3-4)cc21 ; CC1(C)c2ccccc2N(c2c(-c3ccc4ccccc4c3)ccc3ccccc23)c2ccc(N(c3ccc4ccccc4c3)c3cccc4ccccc34)cc21 ; CC1(C)c2ccccc2N(c2c(-c3ccc4ccccc4c3)ccc3ccccc23)c2ccc(N(c3ccc(-c4ccccc4)cc3)c3ccc4c5ccccc5c5ccccc5c4c3)cc21]

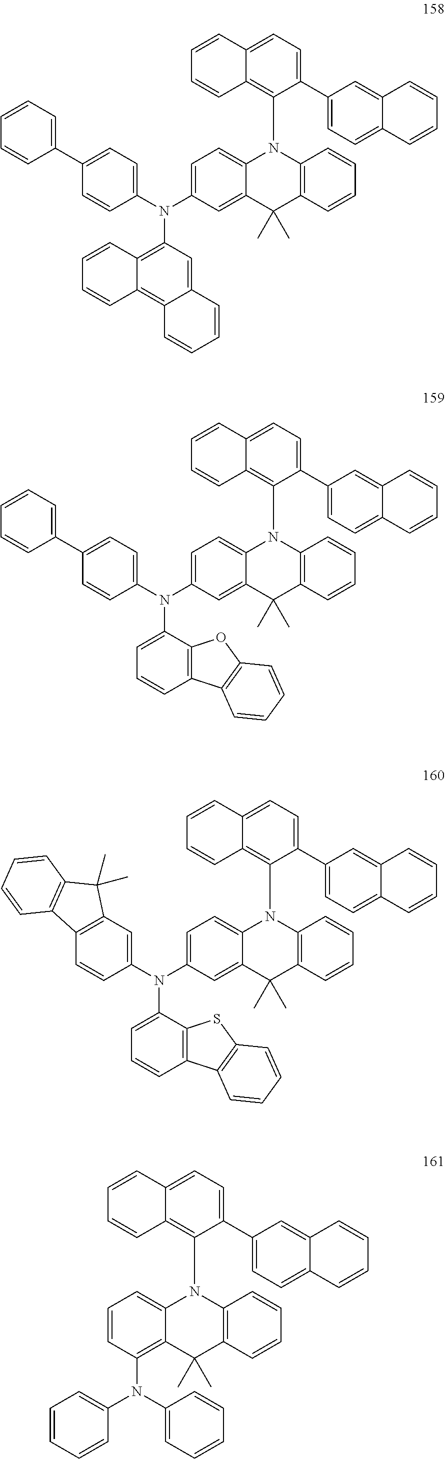 [SMILES: CC1(C)c2ccccc2N(c2c(-c3ccc4ccccc4c3)ccc3ccccc23)c2ccc(N(c3ccc(-c4ccccc4)cc3)c3cc4ccccc4c4ccccc34)cc21 ; CC1(C)c2ccccc2N(c2c(-c3ccc4ccccc4c3)ccc3ccccc23)c2ccc(N(c3ccc(-c4ccccc4)cc3)c3cccc4c3oc3ccccc34)cc21 ; CC1(C)c2ccccc2-c2ccc(N(c3ccc4c(c3)C(C)(C)c3ccccc3N4c3c(-c4ccc5ccccc5c4)ccc4ccccc34)c3cccc4c3sc3ccccc34)cc21 ; CC1(C)c2ccccc2N(c2c(-c3ccc4ccccc4c3)ccc3ccccc23)c2cccc(N(c3ccccc3)c3ccccc3)c21]

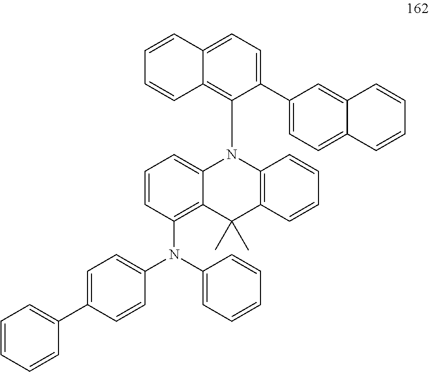 [SMILES: CC1(C)c2ccccc2N(c2c(-c3ccc4ccccc4c3)ccc3ccccc23)c2cccc(N(c3ccccc3)c3ccc(-c4ccccc4)cc3)c21]

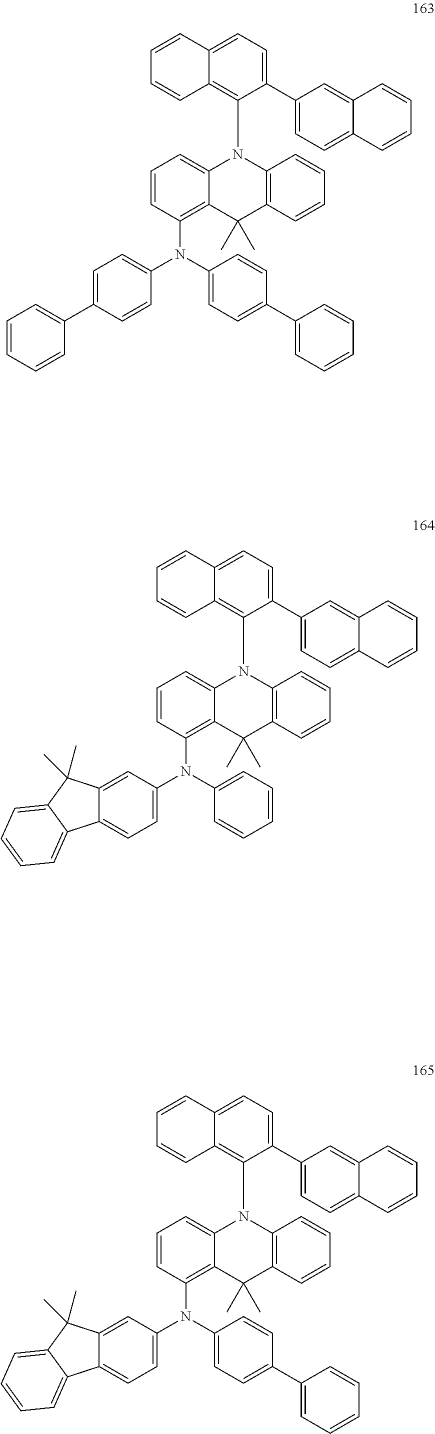 [SMILES: CC1(C)c2ccccc2N(c2c(-c3ccc4ccccc4c3)ccc3ccccc23)c2cccc(N(c3ccc(-c4ccccc4)cc3)c3ccc(-c4ccccc4)cc3)c21 ; CC1(C)c2ccccc2-c2ccc(N(c3ccccc3)c3cccc4c3C(C)(C)c3ccccc3N4c3c(-c4ccc5ccccc5c4)ccc4ccccc34)cc21 ; CC1(C)c2ccccc2-c2ccc(N(c3ccc(-c4ccccc4)cc3)c3cccc4c3C(C)(C)c3ccccc3N4c3c(-c4ccc5ccccc5c4)ccc4ccccc34)cc21]

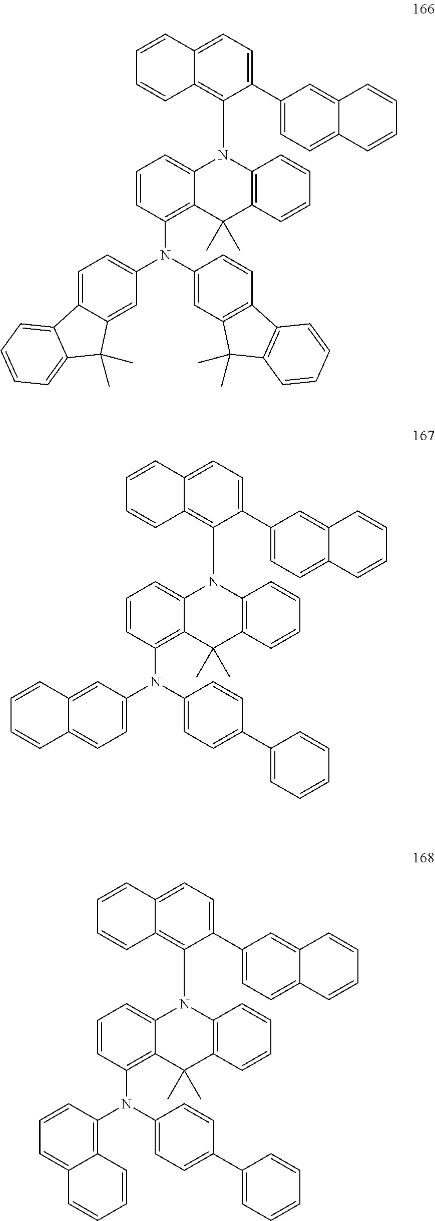 [SMILES: CC1(C)c2ccccc2-c2ccc(N(c3ccc4c(c3)C(C)(C)c3ccccc3-4)c3cccc4c3C(C)(C)c3ccccc3N4c3c(-c4ccc5ccccc5c4)ccc4ccccc34)cc21 ; CC1(C)c2ccccc2N(c2c(-c3ccc4ccccc4c3)ccc3ccccc23)c2cccc(N(c3ccc(-c4ccccc4)cc3)c3ccc4ccccc4c3)c21 ; CC1(C)c2ccccc2N(c2c(-c3ccc4ccccc4c3)ccc3ccccc23)c2cccc(N(c3ccc(-c4ccccc4)cc3)c3cccc4ccccc34)c21]

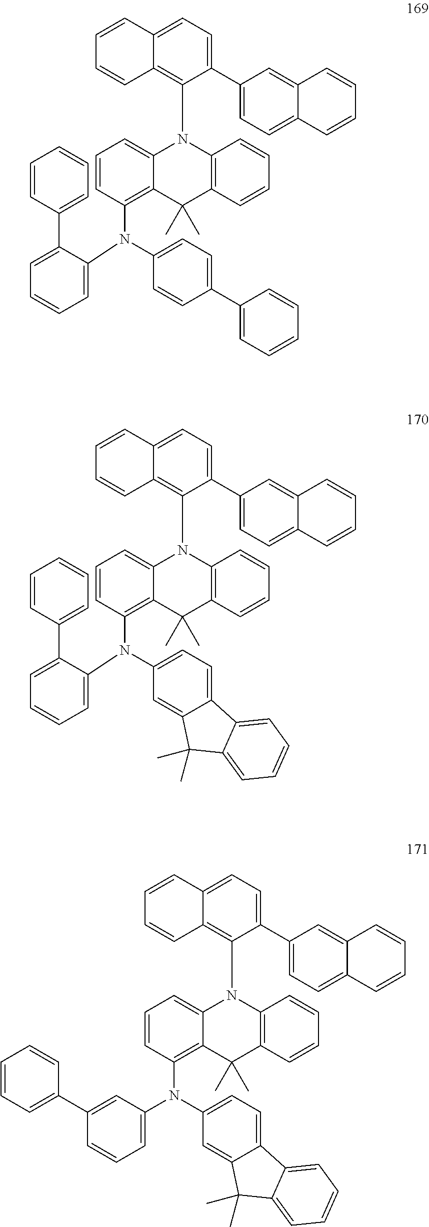 [SMILES: CC1(C)c2ccccc2N(c2c(-c3ccc4ccccc4c3)ccc3ccccc23)c2cccc(N(c3ccc(-c4ccccc4)cc3)c3ccccc3-c3ccccc3)c21 ; CC1(C)c2ccccc2-c2ccc(N(c3ccccc3-c3ccccc3)c3cccc4c3C(C)(C)c3ccccc3N4c3c(-c4ccc5ccccc5c4)ccc4ccccc34)cc21 ; CC1(C)c2ccccc2-c2ccc(N(c3cccc(-c4ccccc4)c3)c3cccc4c3C(C)(C)c3ccccc3N4c3c(-c4ccc5ccccc5c4)ccc4ccccc34)cc21]

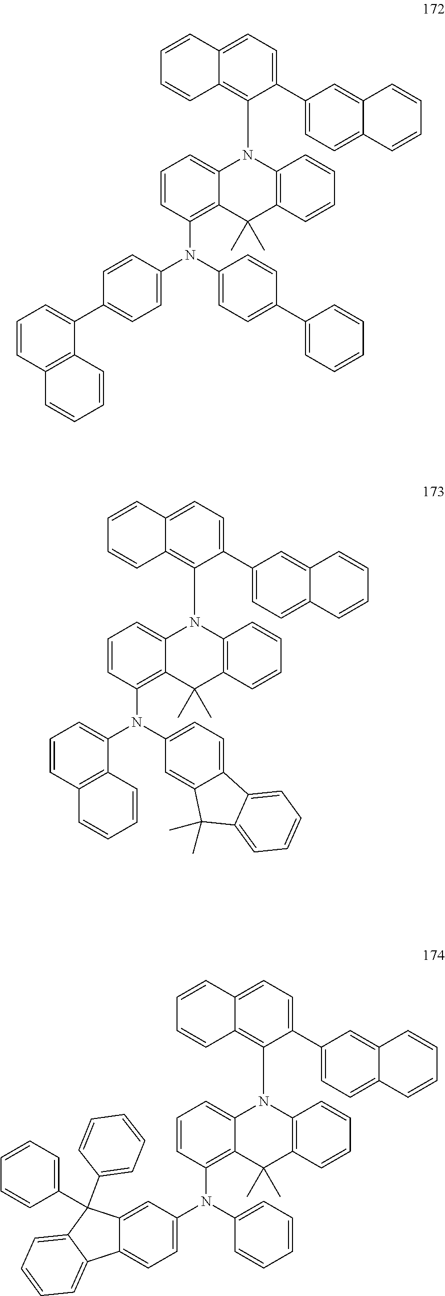 [SMILES: CC1(C)c2ccccc2N(c2c(-c3ccc4ccccc4c3)ccc3ccccc23)c2cccc(N(c3ccc(-c4ccccc4)cc3)c3ccc(-c4cccc5ccccc45)cc3)c21 ; CC1(C)c2ccccc2-c2ccc(N(c3cccc4c3C(C)(C)c3ccccc3N4c3c(-c4ccc5ccccc5c4)ccc4ccccc34)c3cccc4ccccc34)cc21 ; CC1(C)c2ccccc2N(c2c(-c3ccc4ccccc4c3)ccc3ccccc23)c2cccc(N(c3ccccc3)c3ccc4c(c3)C(c3ccccc3)(c3ccccc3)c3ccccc3-4)c21]

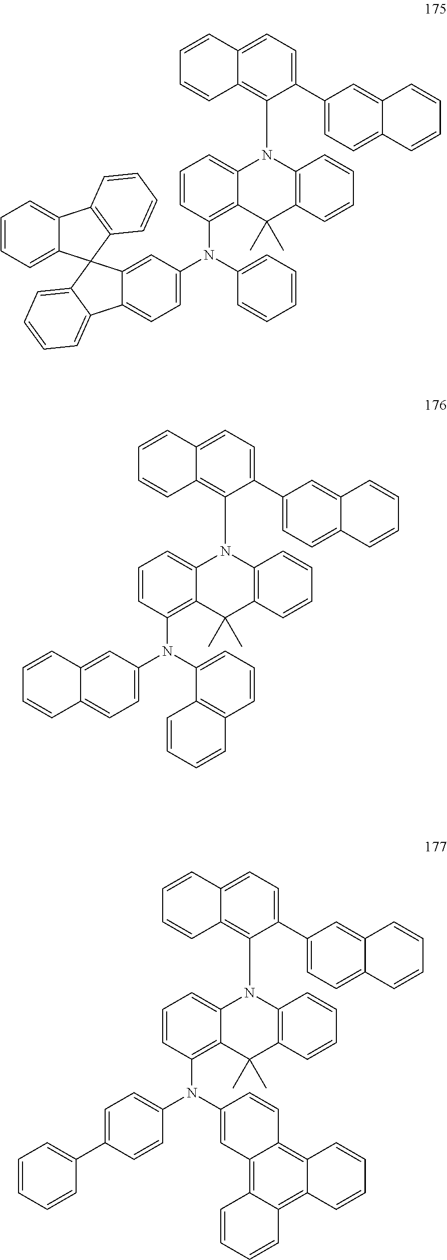 [SMILES: CC1(C)c2ccccc2N(c2c(-c3ccc4ccccc4c3)ccc3ccccc23)c2cccc(N(c3ccccc3)c3ccc4c(c3)C3(c5ccccc5-c5ccccc53)c3ccccc3-4)c21 ; CC1(C)c2ccccc2N(c2c(-c3ccc4ccccc4c3)ccc3ccccc23)c2cccc(N(c3ccc4ccccc4c3)c3cccc4ccccc34)c21 ; CC1(C)c2ccccc2N(c2c(-c3ccc4ccccc4c3)ccc3ccccc23)c2cccc(N(c3ccc(-c4ccccc4)cc3)c3ccc4c5ccccc5c5ccccc5c4c3)c21]

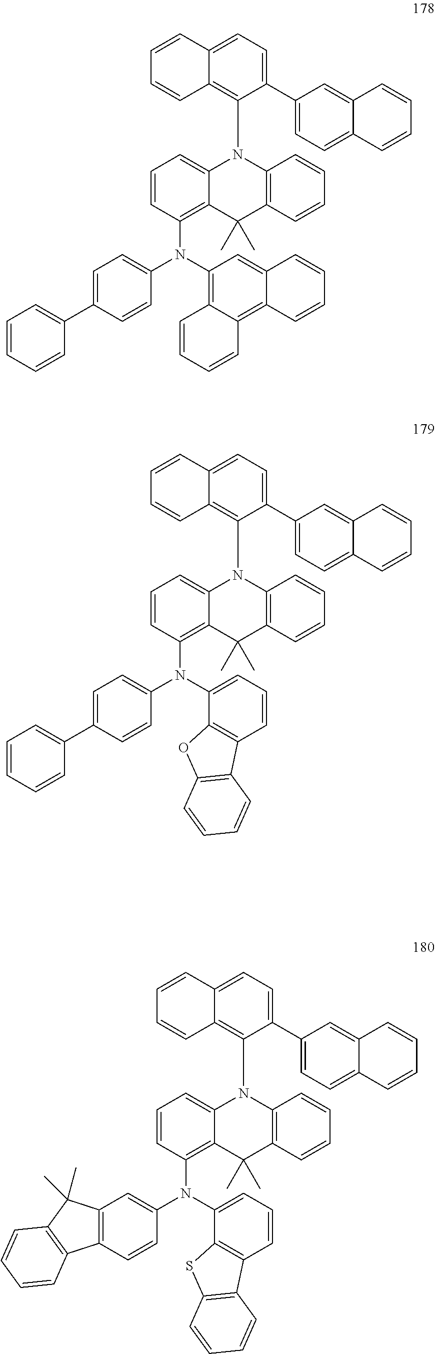 [SMILES: CC1(C)c2ccccc2N(c2c(-c3ccc4ccccc4c3)ccc3ccccc23)c2cccc(N(c3ccc(-c4ccccc4)cc3)c3cc4ccccc4c4ccccc34)c21 ; CC1(C)c2ccccc2N(c2c(-c3ccc4ccccc4c3)ccc3ccccc23)c2cccc(N(c3ccc(-c4ccccc4)cc3)c3cccc4c3oc3ccccc34)c21 ; CC1(C)c2ccccc2-c2ccc(N(c3cccc4c3C(C)(C)c3ccccc3N4c3c(-c4ccc5ccccc5c4)ccc4ccccc34)c3cccc4c3sc3ccccc34)cc21]

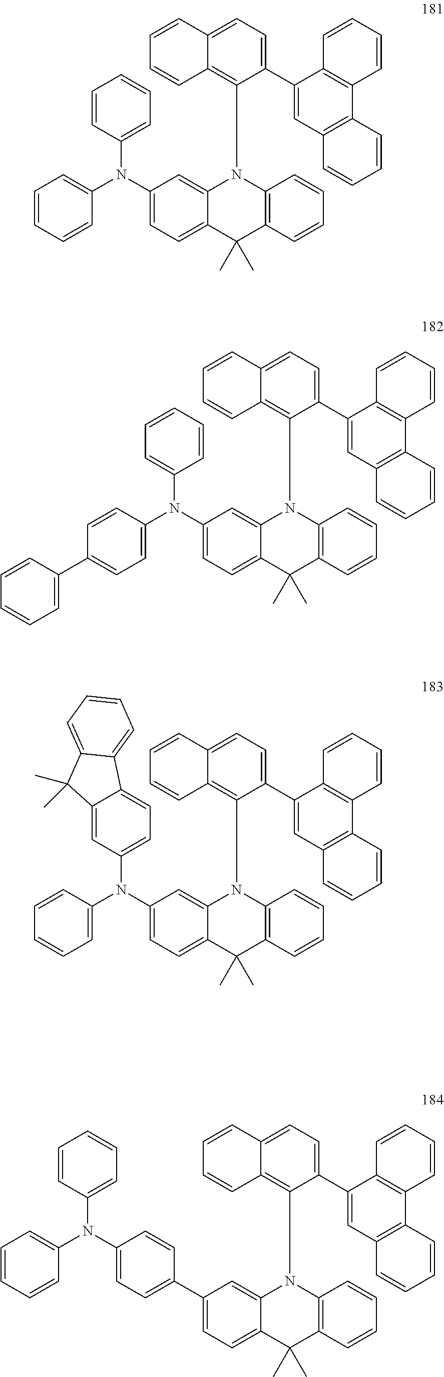 [SMILES: CC1(C)c2ccccc2N(c2c(-c3cc4ccccc4c4ccccc34)ccc3ccccc23)c2cc(N(c3ccccc3)c3ccccc3)ccc21 ; CC1(C)c2ccccc2N(c2c(-c3cc4ccccc4c4ccccc34)ccc3ccccc23)c2cc(N(c3ccccc3)c3ccc(-c4ccccc4)cc3)ccc21 ; CC1(C)c2ccccc2-c2ccc(N(c3ccccc3)c3ccc4c(c3)N(c3c(-c5cc6ccccc6c6ccccc56)ccc5ccccc35)c3ccccc3C4(C)C)cc21 ; CC1(C)c2ccccc2N(c2c(-c3cc4ccccc4c4ccccc34)ccc3ccccc23)c2cc(-c3ccc(N(c4ccccc4)c4ccccc4)cc3)ccc21]

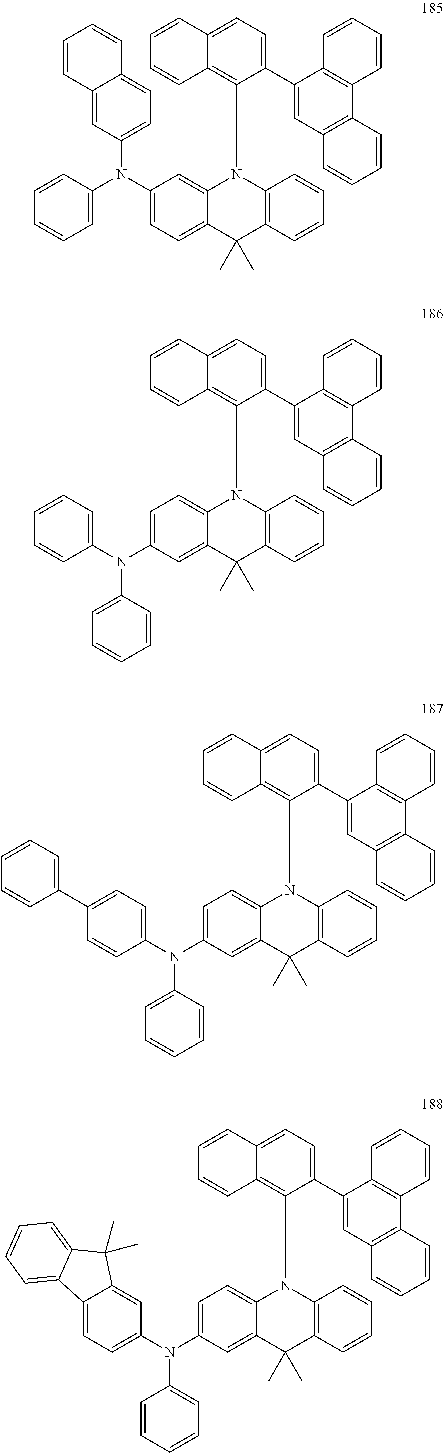 [SMILES: CC1(C)c2ccccc2N(c2c(-c3cc4ccccc4c4ccccc34)ccc3ccccc23)c2cc(N(c3ccccc3)c3ccc4ccccc4c3)ccc21 ; CC1(C)c2ccccc2N(c2c(-c3cc4ccccc4c4ccccc34)ccc3ccccc23)c2ccc(N(c3ccccc3)c3ccccc3)cc21 ; CC1(C)c2ccccc2N(c2c(-c3cc4ccccc4c4ccccc34)ccc3ccccc23)c2ccc(N(c3ccccc3)c3ccc(-c4ccccc4)cc3)cc21 ; CC1(C)c2ccccc2-c2ccc(N(c3ccccc3)c3ccc4c(c3)C(C)(C)c3ccccc3N4c3c(-c4cc5ccccc5c5ccccc45)ccc4ccccc34)cc21]

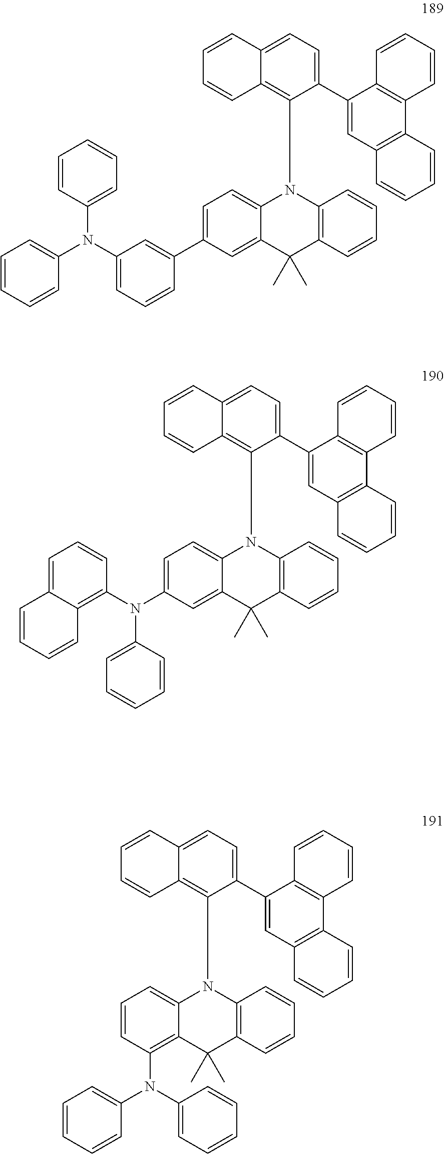 [SMILES: CC1(C)c2ccccc2N(c2c(-c3cc4ccccc4c4ccccc34)ccc3ccccc23)c2ccc(-c3cccc(N(c4ccccc4)c4ccccc4)c3)cc21 ; CC1(C)c2ccccc2N(c2c(-c3cc4ccccc4c4ccccc34)ccc3ccccc23)c2ccc(N(c3ccccc3)c3cccc4ccccc34)cc21 ; CC1(C)c2ccccc2N(c2c(-c3cc4ccccc4c4ccccc34)ccc3ccccc23)c2cccc(N(c3ccccc3)c3ccccc3)c21]

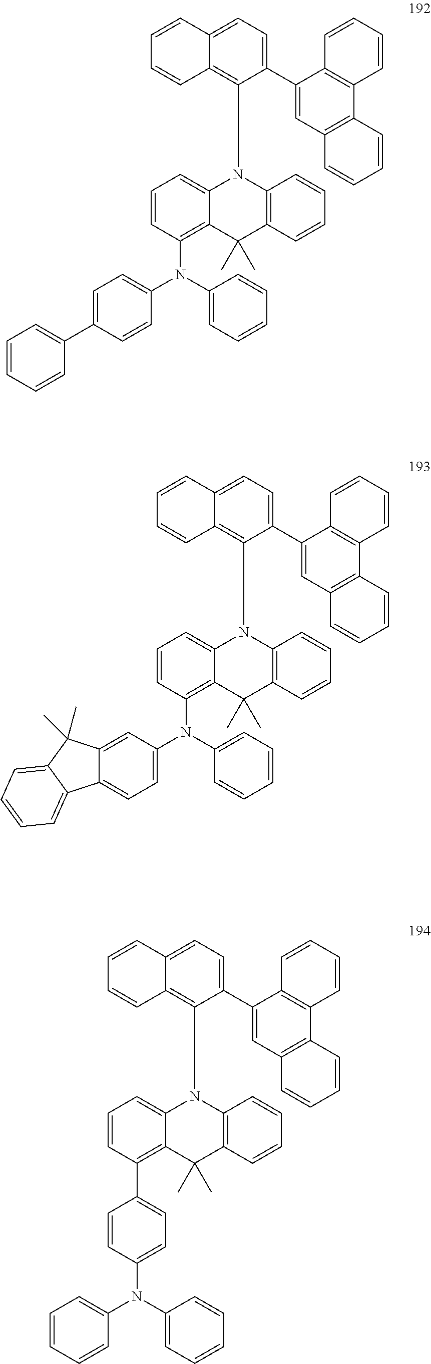 [SMILES: CC1(C)c2ccccc2N(c2c(-c3cc4ccccc4c4ccccc34)ccc3ccccc23)c2cccc(N(c3ccccc3)c3ccc(-c4ccccc4)cc3)c21 ; CC1(C)c2ccccc2-c2ccc(N(c3ccccc3)c3cccc4c3C(C)(C)c3ccccc3N4c3c(-c4cc5ccccc5c5ccccc45)ccc4ccccc34)cc21 ; CC1(C)c2ccccc2N(c2c(-c3cc4ccccc4c4ccccc34)ccc3ccccc23)c2cccc(-c3ccc(N(c4ccccc4)c4ccccc4)cc3)c21]

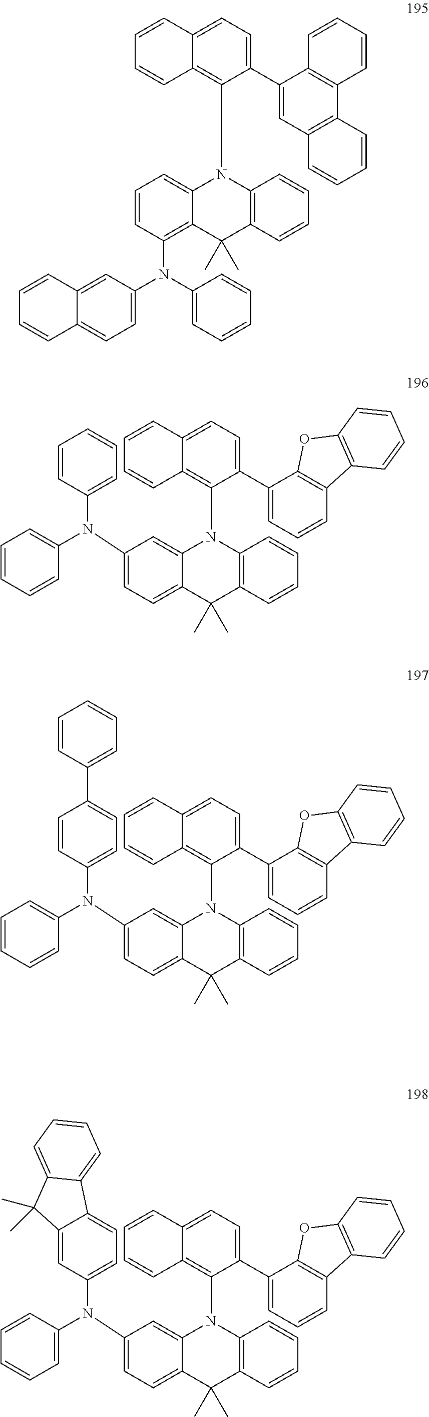 [SMILES: CC1(C)c2ccccc2N(c2c(-c3cc4ccccc4c4ccccc34)ccc3ccccc23)c2cccc(N(c3ccccc3)c3ccc4ccccc4c3)c21 ; CC1(C)c2ccccc2N(c2c(-c3cccc4c3oc3ccccc34)ccc3ccccc23)c2cc(N(c3ccccc3)c3ccccc3)ccc21 ; CC1(C)c2ccccc2N(c2c(-c3cccc4c3oc3ccccc34)ccc3ccccc23)c2cc(N(c3ccccc3)c3ccc(-c4ccccc4)cc3)ccc21 ; CC1(C)c2ccccc2-c2ccc(N(c3ccccc3)c3ccc4c(c3)N(c3c(-c5cccc6c5oc5ccccc56)ccc5ccccc35)c3ccccc3C4(C)C)cc21]

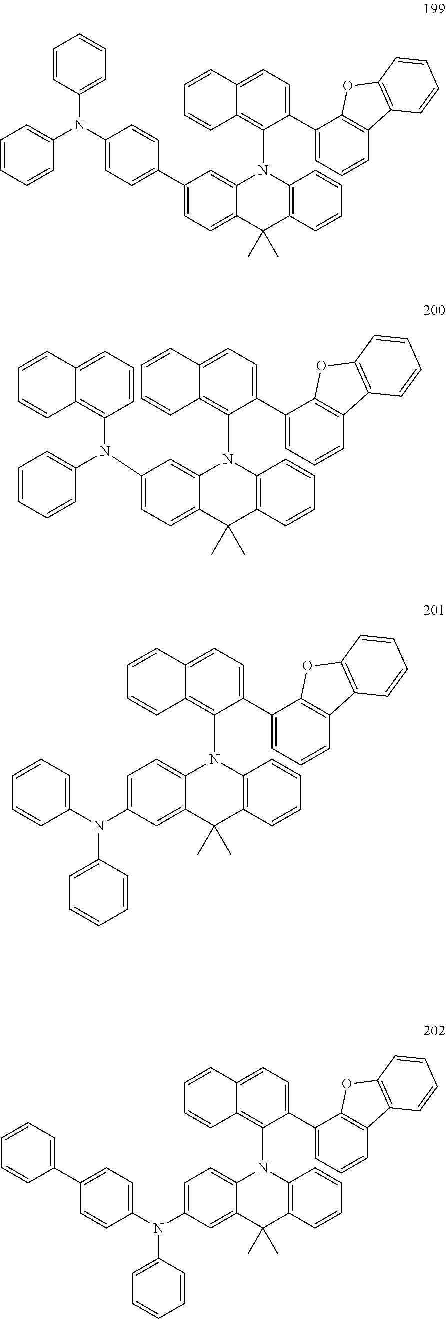 [SMILES: CC1(C)c2ccccc2N(c2c(-c3cccc4c3oc3ccccc34)ccc3ccccc23)c2cc(-c3ccc(N(c4ccccc4)c4ccccc4)cc3)ccc21 ; CC1(C)c2ccccc2N(c2c(-c3cccc4c3oc3ccccc34)ccc3ccccc23)c2cc(N(c3ccccc3)c3cccc4ccccc34)ccc21 ; CC1(C)c2ccccc2N(c2c(-c3cccc4c3oc3ccccc34)ccc3ccccc23)c2ccc(N(c3ccccc3)c3ccccc3)cc21 ; CC1(C)c2ccccc2N(c2c(-c3cccc4c3oc3ccccc34)ccc3ccccc23)c2ccc(N(c3ccccc3)c3ccc(-c4ccccc4)cc3)cc21]

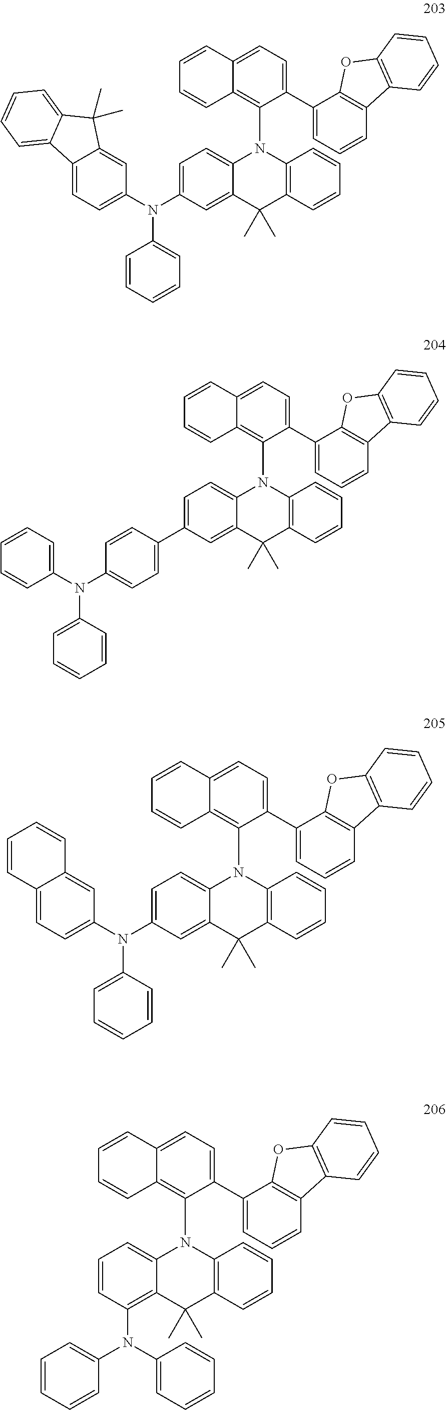 [SMILES: CC1(C)c2ccccc2-c2ccc(N(c3ccccc3)c3ccc4c(c3)C(C)(C)c3ccccc3N4c3c(-c4cccc5c4oc4ccccc45)ccc4ccccc34)cc21 ; CC1(C)c2ccccc2N(c2c(-c3cccc4c3oc3ccccc34)ccc3ccccc23)c2ccc(-c3ccc(N(c4ccccc4)c4ccccc4)cc3)cc21 ; CC1(C)c2ccccc2N(c2c(-c3cccc4c3oc3ccccc34)ccc3ccccc23)c2ccc(N(c3ccccc3)c3ccc4ccccc4c3)cc21 ; CC1(C)c2ccccc2N(c2c(-c3cccc4c3oc3ccccc34)ccc3ccccc23)c2cccc(N(c3ccccc3)c3ccccc3)c21]

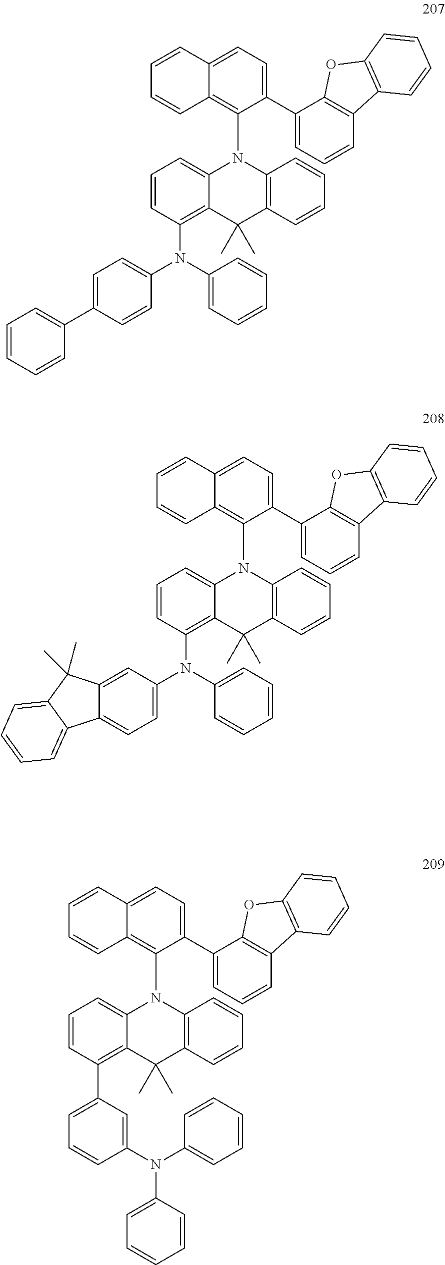 [SMILES: CC1(C)c2ccccc2N(c2c(-c3cccc4c3oc3ccccc34)ccc3ccccc23)c2cccc(N(c3ccccc3)c3ccc(-c4ccccc4)cc3)c21 ; CC1(C)c2ccccc2-c2ccc(N(c3ccccc3)c3cccc4c3C(C)(C)c3ccccc3N4c3c(-c4cccc5c4oc4ccccc45)ccc4ccccc34)cc21 ; CC1(C)c2ccccc2N(c2c(-c3cccc4c3oc3ccccc34)ccc3ccccc23)c2cccc(-c3cccc(N(c4ccccc4)c4ccccc4)c3)c21]

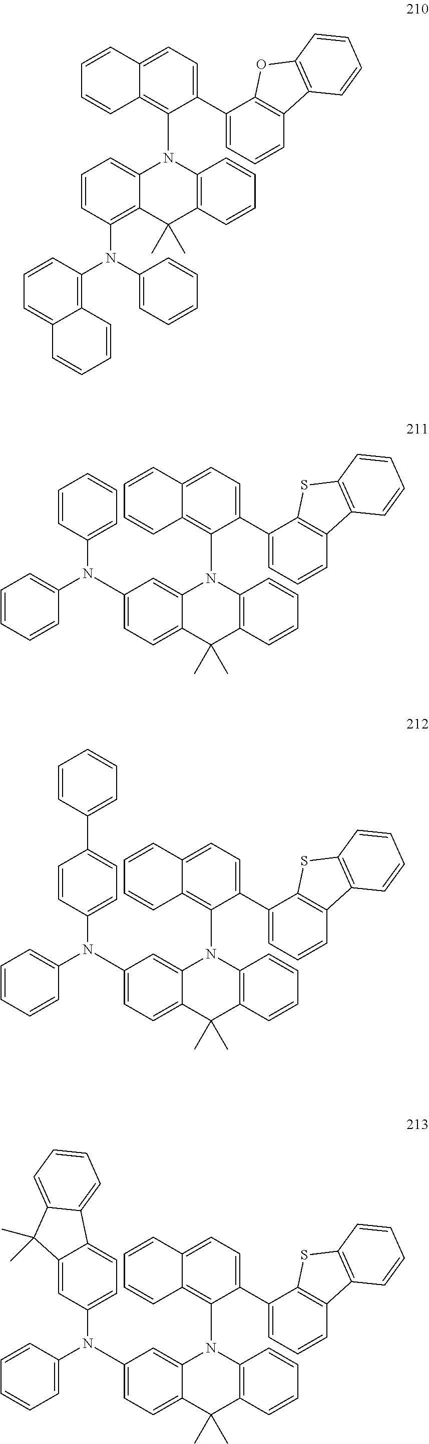 [SMILES: CC1(C)c2ccccc2N(c2c(-c3cccc4c3oc3ccccc34)ccc3ccccc23)c2cccc(N(c3ccccc3)c3cccc4ccccc34)c21 ; CC1(C)c2ccccc2N(c2c(-c3cccc4c3sc3ccccc34)ccc3ccccc23)c2cc(N(c3ccccc3)c3ccccc3)ccc21 ; CC1(C)c2ccccc2N(c2c(-c3cccc4c3sc3ccccc34)ccc3ccccc23)c2cc(N(c3ccccc3)c3ccc(-c4ccccc4)cc3)ccc21 ; CC1(C)c2ccccc2-c2ccc(N(c3ccccc3)c3ccc4c(c3)N(c3c(-c5cccc6c5sc5ccccc56)ccc5ccccc35)c3ccccc3C4(C)C)cc21]

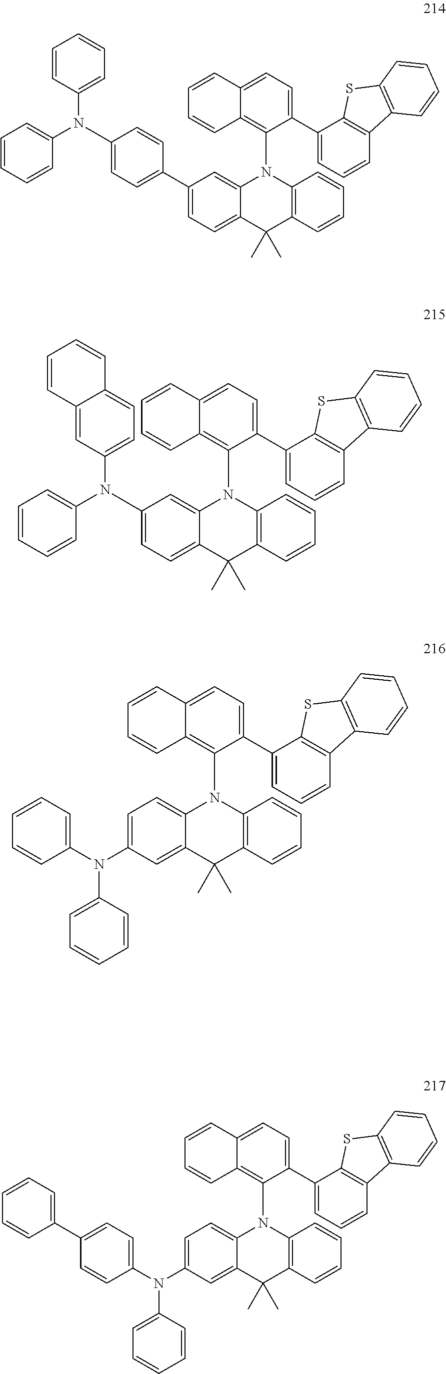 [SMILES: CC1(C)c2ccccc2N(c2c(-c3cccc4c3sc3ccccc34)ccc3ccccc23)c2cc(-c3ccc(N(c4ccccc4)c4ccccc4)cc3)ccc21 ; CC1(C)c2ccccc2N(c2c(-c3cccc4c3sc3ccccc34)ccc3ccccc23)c2cc(N(c3ccccc3)c3ccc4ccccc4c3)ccc21 ; CC1(C)c2ccccc2N(c2c(-c3cccc4c3sc3ccccc34)ccc3ccccc23)c2ccc(N(c3ccccc3)c3ccccc3)cc21 ; CC1(C)c2ccccc2N(c2c(-c3cccc4c3sc3ccccc34)ccc3ccccc23)c2ccc(N(c3ccccc3)c3ccc(-c4ccccc4)cc3)cc21]

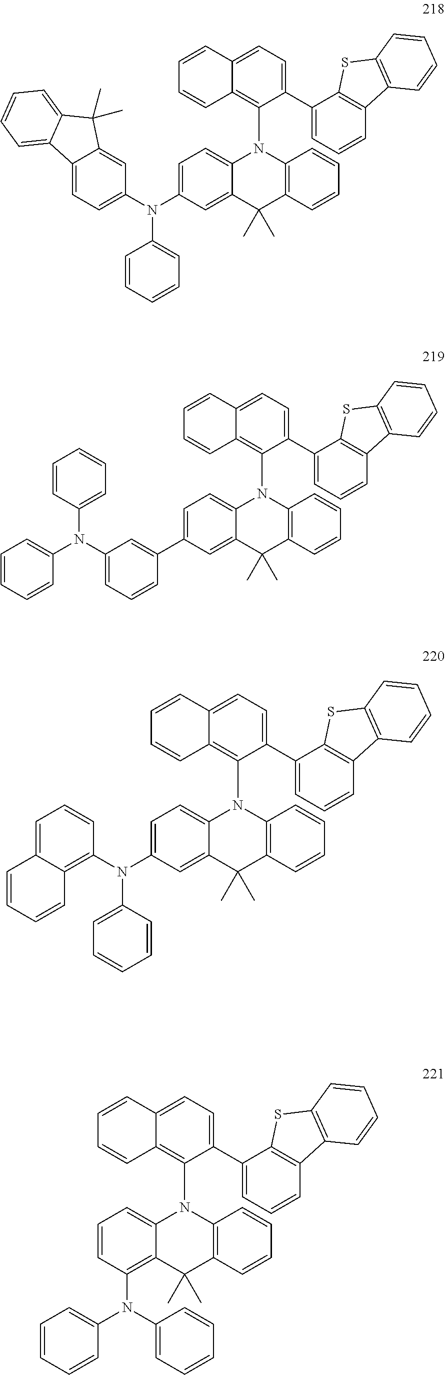 [SMILES: CC1(C)c2ccccc2-c2ccc(N(c3ccccc3)c3ccc4c(c3)C(C)(C)c3ccccc3N4c3c(-c4cccc5c4sc4ccccc45)ccc4ccccc34)cc21 ; CC1(C)c2ccccc2N(c2c(-c3cccc4c3sc3ccccc34)ccc3ccccc23)c2ccc(-c3cccc(N(c4ccccc4)c4ccccc4)c3)cc21 ; CC1(C)c2ccccc2N(c2c(-c3cccc4c3sc3ccccc34)ccc3ccccc23)c2ccc(N(c3ccccc3)c3cccc4ccccc34)cc21 ; CC1(C)c2ccccc2N(c2c(-c3cccc4c3sc3ccccc34)ccc3ccccc23)c2cccc(N(c3ccccc3)c3ccccc3)c21]

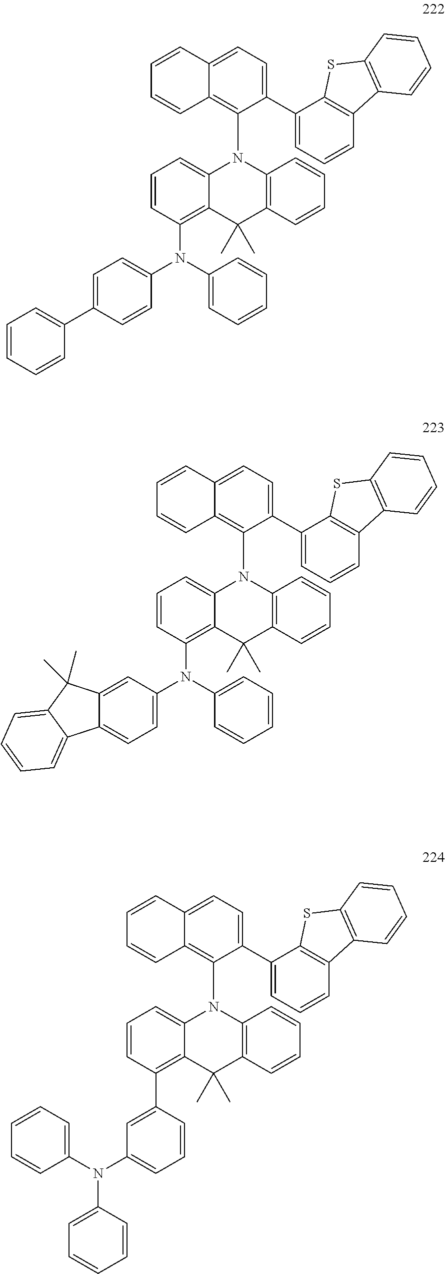 [SMILES: CC1(C)c2ccccc2N(c2c(-c3cccc4c3sc3ccccc34)ccc3ccccc23)c2cccc(N(c3ccccc3)c3ccc(-c4ccccc4)cc3)c21 ; CC1(C)c2ccccc2-c2ccc(N(c3ccccc3)c3cccc4c3C(C)(C)c3ccccc3N4c3c(-c4cccc5c4sc4ccccc45)ccc4ccccc34)cc21 ; CC1(C)c2ccccc2N(c2c(-c3cccc4c3sc3ccccc34)ccc3ccccc23)c2cccc(-c3cccc(N(c4ccccc4)c4ccccc4)c3)c21]

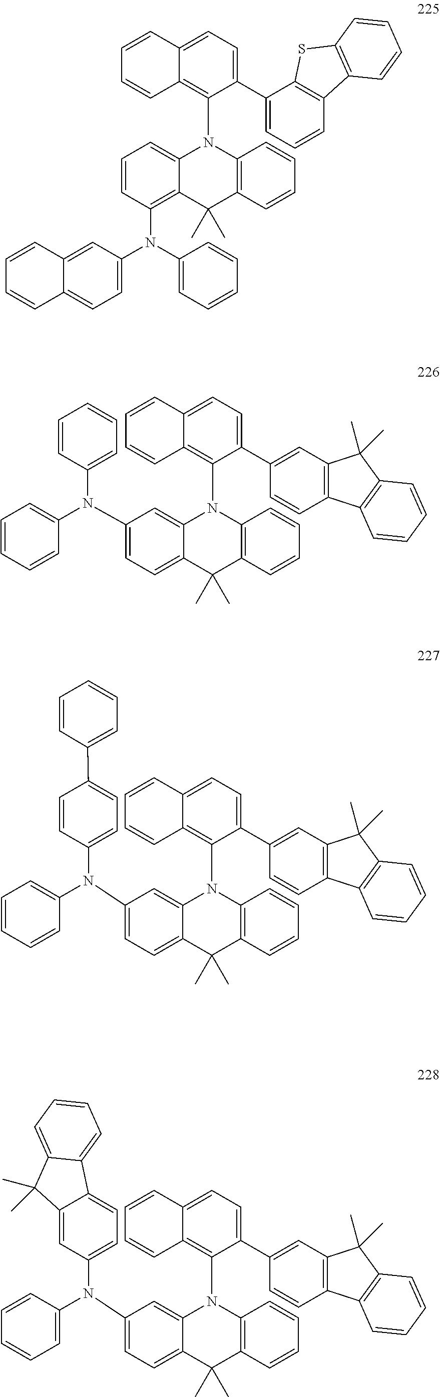 [SMILES: CC1(C)c2ccccc2N(c2c(-c3cccc4c3sc3ccccc34)ccc3ccccc23)c2cccc(N(c3ccccc3)c3ccc4ccccc4c3)c21 ; CC1(C)c2ccccc2-c2ccc(-c3ccc4ccccc4c3N3c4ccccc4C(C)(C)c4ccc(N(c5ccccc5)c5ccccc5)cc43)cc21 ; CC1(C)c2ccccc2-c2ccc(-c3ccc4ccccc4c3N3c4ccccc4C(C)(C)c4ccc(N(c5ccccc5)c5ccc(-c6ccccc6)cc5)cc43)cc21 ; CC1(C)c2ccccc2-c2ccc(-c3ccc4ccccc4c3N3c4ccccc4C(C)(C)c4ccc(N(c5ccccc5)c5ccc6c(c5)C(C)(C)c5ccccc5-6)cc43)cc21]

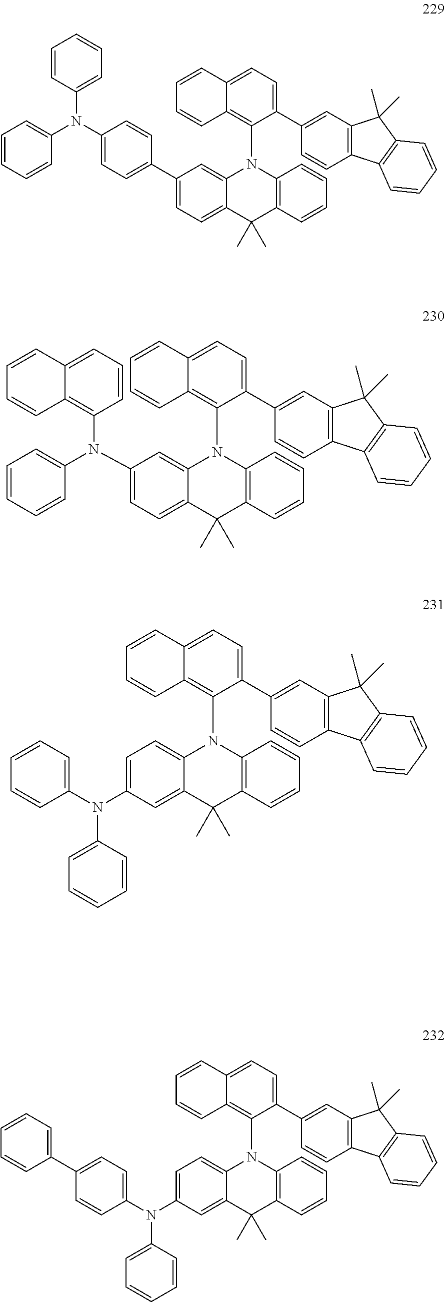 [SMILES: CC1(C)c2ccccc2-c2ccc(-c3ccc4ccccc4c3N3c4ccccc4C(C)(C)c4ccc(-c5ccc(N(c6ccccc6)c6ccccc6)cc5)cc43)cc21 ; CC1(C)c2ccccc2-c2ccc(-c3ccc4ccccc4c3N3c4ccccc4C(C)(C)c4ccc(N(c5ccccc5)c5cccc6ccccc56)cc43)cc21 ; CC1(C)c2ccccc2-c2ccc(-c3ccc4ccccc4c3N3c4ccccc4C(C)(C)c4cc(N(c5ccccc5)c5ccccc5)ccc43)cc21 ; CC1(C)c2ccccc2-c2ccc(-c3ccc4ccccc4c3N3c4ccccc4C(C)(C)c4cc(N(c5ccccc5)c5ccc(-c6ccccc6)cc5)ccc43)cc21]

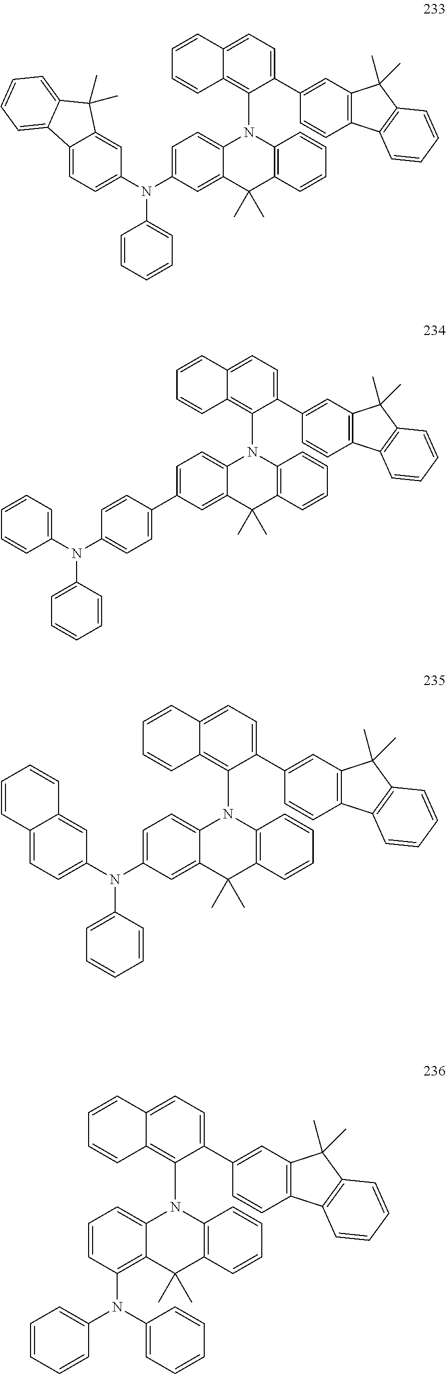 [SMILES: CC1(C)c2ccccc2-c2ccc(-c3ccc4ccccc4c3N3c4ccccc4C(C)(C)c4cc(N(c5ccccc5)c5ccc6c(c5)C(C)(C)c5ccccc5-6)ccc43)cc21 ; CC1(C)c2ccccc2-c2ccc(-c3ccc4ccccc4c3N3c4ccccc4C(C)(C)c4cc(-c5ccc(N(c6ccccc6)c6ccccc6)cc5)ccc43)cc21 ; CC1(C)c2ccccc2-c2ccc(-c3ccc4ccccc4c3N3c4ccccc4C(C)(C)c4cc(N(c5ccccc5)c5ccc6ccccc6c5)ccc43)cc21 ; CC1(C)c2ccccc2-c2ccc(-c3ccc4ccccc4c3N3c4ccccc4C(C)(C)c4c(N(c5ccccc5)c5ccccc5)cccc43)cc21]

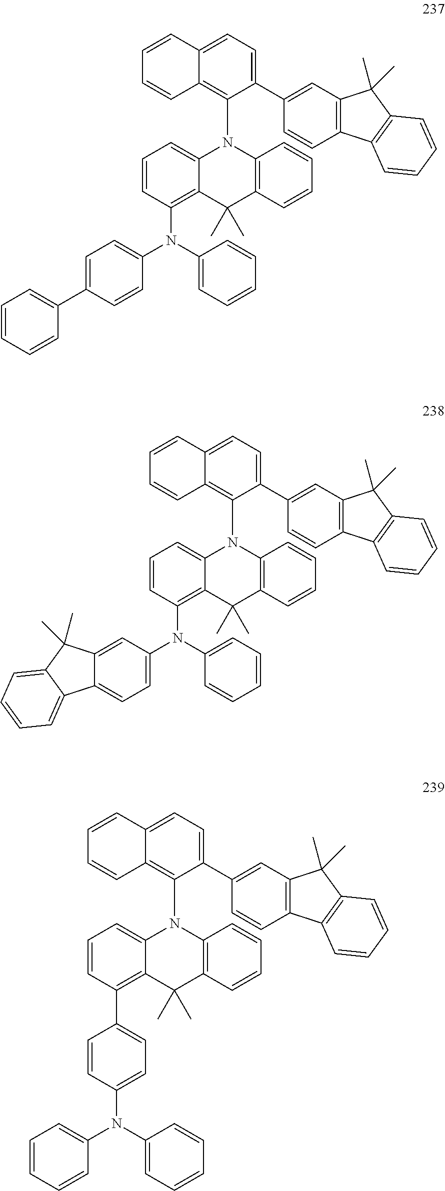 [SMILES: CC1(C)c2ccccc2-c2ccc(-c3ccc4ccccc4c3N3c4ccccc4C(C)(C)c4c(N(c5ccccc5)c5ccc(-c6ccccc6)cc5)cccc43)cc21 ; CC1(C)c2ccccc2-c2ccc(-c3ccc4ccccc4c3N3c4ccccc4C(C)(C)c4c(N(c5ccccc5)c5ccc6c(c5)C(C)(C)c5ccccc5-6)cccc43)cc21 ; CC1(C)c2ccccc2-c2ccc(-c3ccc4ccccc4c3N3c4ccccc4C(C)(C)c4c(-c5ccc(N(c6ccccc6)c6ccccc6)cc5)cccc43)cc21]

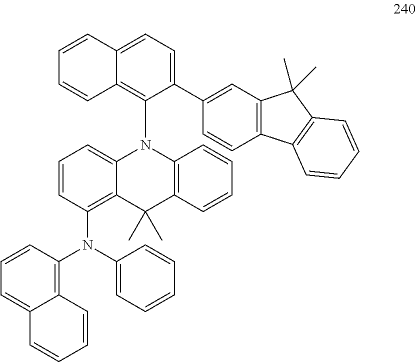 [SMILES: CC1(C)c2ccccc2-c2ccc(-c3ccc4ccccc4c3N3c4ccccc4C(C)(C)c4c(N(c5ccccc5)c5cccc6ccccc56)cccc43)cc21]

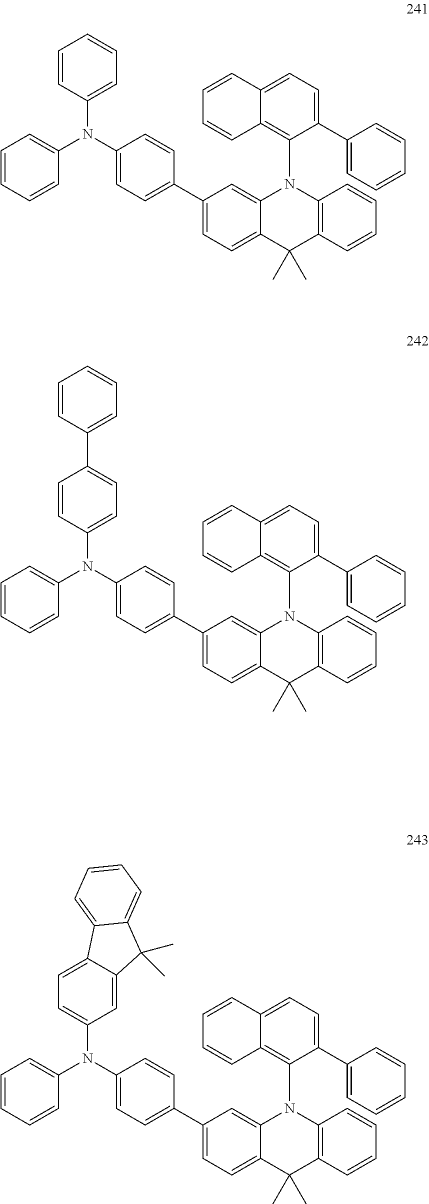 [SMILES: CC1(C)c2ccccc2N(c2c(-c3ccccc3)ccc3ccccc23)c2cc(-c3ccc(N(c4ccccc4)c4ccccc4)cc3)ccc21 ; CC1(C)c2ccccc2N(c2c(-c3ccccc3)ccc3ccccc23)c2cc(-c3ccc(N(c4ccccc4)c4ccc(-c5ccccc5)cc4)cc3)ccc21 ; CC1(C)c2ccccc2-c2ccc(N(c3ccccc3)c3ccc(-c4ccc5c(c4)N(c4c(-c6ccccc6)ccc6ccccc46)c4ccccc4C5(C)C)cc3)cc21]

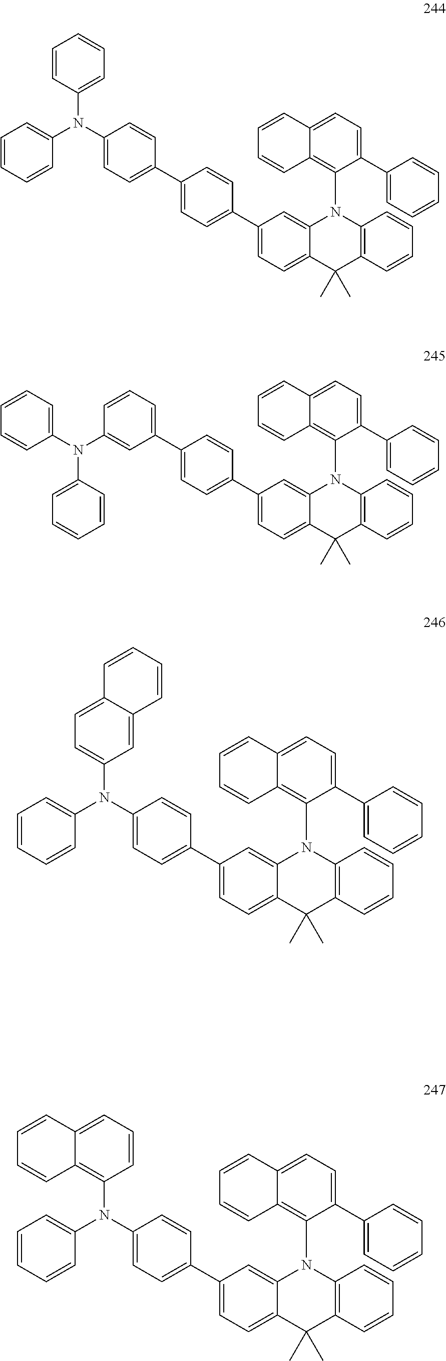 [SMILES: CC1(C)c2ccccc2N(c2c(-c3ccccc3)ccc3ccccc23)c2cc(-c3ccc(-c4ccc(N(c5ccccc5)c5ccccc5)cc4)cc3)ccc21 ; CC1(C)c2ccccc2N(c2c(-c3ccccc3)ccc3ccccc23)c2cc(-c3ccc(-c4cccc(N(c5ccccc5)c5ccccc5)c4)cc3)ccc21 ; CC1(C)c2ccccc2N(c2c(-c3ccccc3)ccc3ccccc23)c2cc(-c3ccc(N(c4ccccc4)c4ccc5ccccc5c4)cc3)ccc21 ; CC1(C)c2ccccc2N(c2c(-c3ccccc3)ccc3ccccc23)c2cc(-c3ccc(N(c4ccccc4)c4cccc5ccccc45)cc3)ccc21]

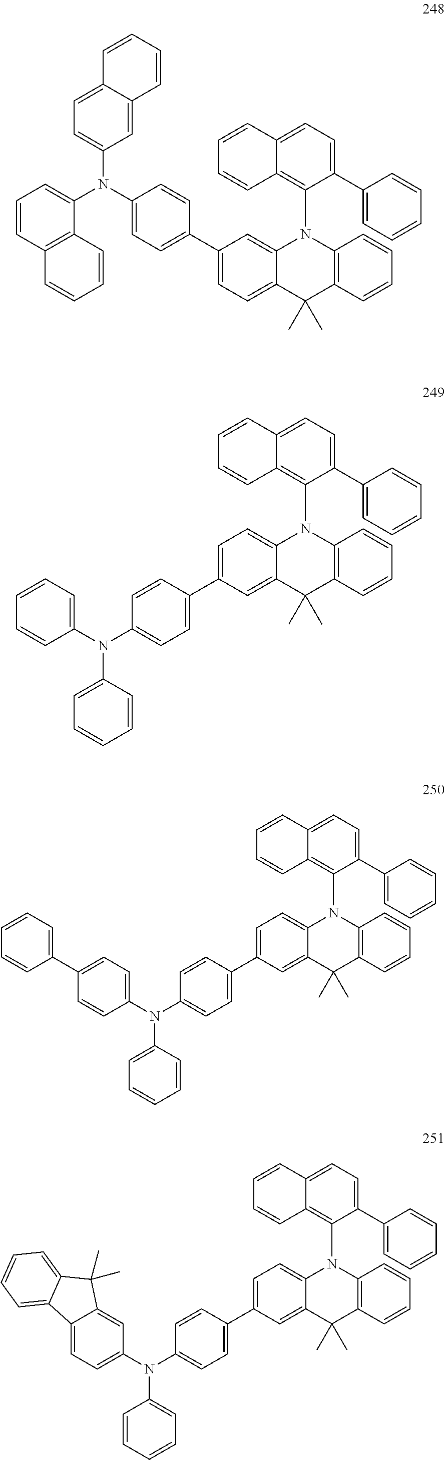 [SMILES: CC1(C)c2ccccc2N(c2c(-c3ccccc3)ccc3ccccc23)c2cc(-c3ccc(N(c4ccc5ccccc5c4)c4cccc5ccccc45)cc3)ccc21 ; CC1(C)c2ccccc2N(c2c(-c3ccccc3)ccc3ccccc23)c2ccc(-c3ccc(N(c4ccccc4)c4ccccc4)cc3)cc21 ; CC1(C)c2ccccc2N(c2c(-c3ccccc3)ccc3ccccc23)c2ccc(-c3ccc(N(c4ccccc4)c4ccc(-c5ccccc5)cc4)cc3)cc21 ; CC1(C)c2ccccc2-c2ccc(N(c3ccccc3)c3ccc(-c4ccc5c(c4)C(C)(C)c4ccccc4N5c4c(-c5ccccc5)ccc5ccccc45)cc3)cc21]

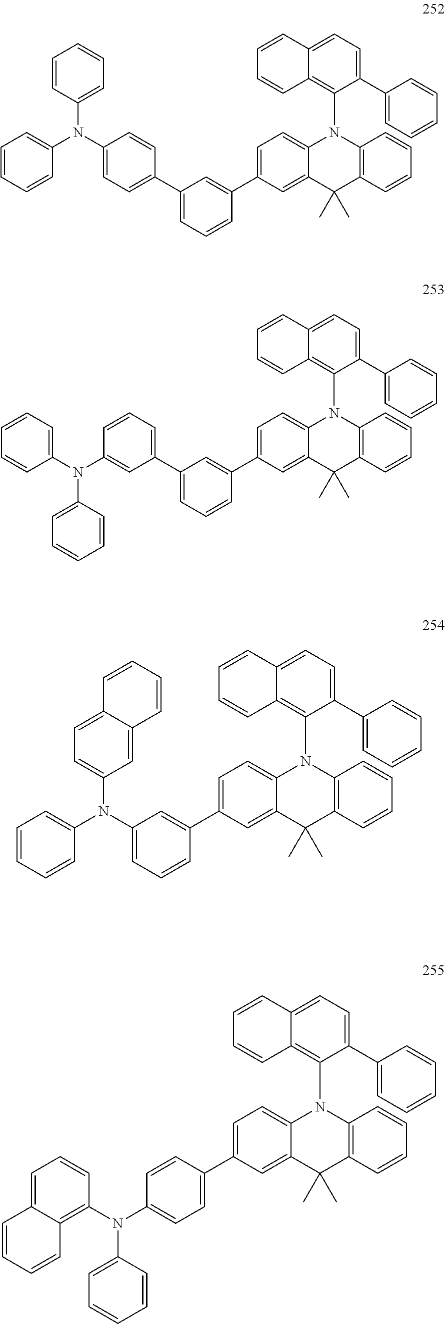 [SMILES: CC1(C)c2ccccc2N(c2c(-c3ccccc3)ccc3ccccc23)c2ccc(-c3cccc(-c4ccc(N(c5ccccc5)c5ccccc5)cc4)c3)cc21 ; CC1(C)c2ccccc2N(c2c(-c3ccccc3)ccc3ccccc23)c2ccc(-c3cccc(-c4cccc(N(c5ccccc5)c5ccccc5)c4)c3)cc21 ; CC1(C)c2ccccc2N(c2c(-c3ccccc3)ccc3ccccc23)c2ccc(-c3cccc(N(c4ccccc4)c4ccc5ccccc5c4)c3)cc21 ; CC1(C)c2ccccc2N(c2c(-c3ccccc3)ccc3ccccc23)c2ccc(-c3ccc(N(c4ccccc4)c4cccc5ccccc45)cc3)cc21]

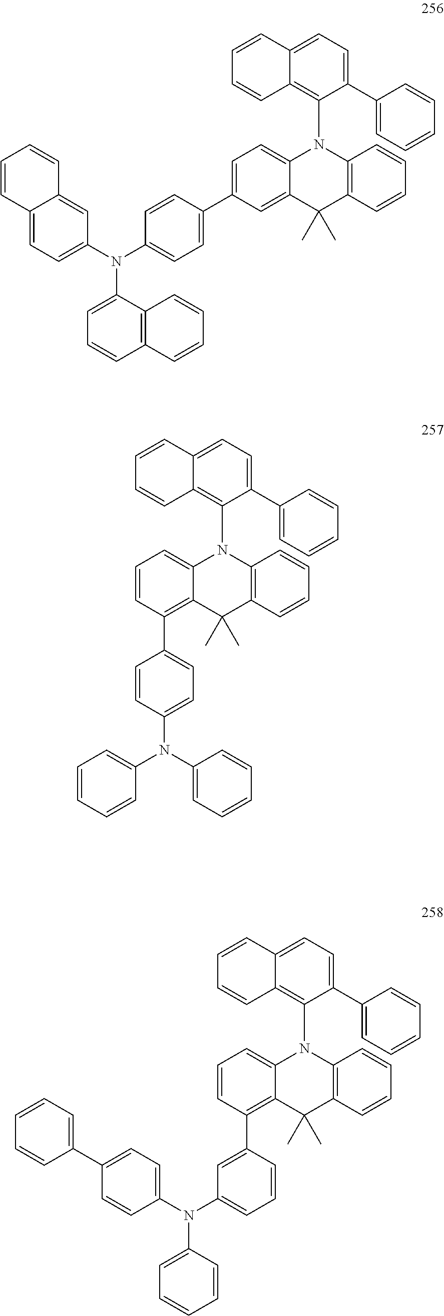 [SMILES: CC1(C)c2ccccc2N(c2c(-c3ccccc3)ccc3ccccc23)c2ccc(-c3ccc(N(c4ccc5ccccc5c4)c4cccc5ccccc45)cc3)cc21 ; CC1(C)c2ccccc2N(c2c(-c3ccccc3)ccc3ccccc23)c2cccc(-c3ccc(N(c4ccccc4)c4ccccc4)cc3)c21 ; CC1(C)c2ccccc2N(c2c(-c3ccccc3)ccc3ccccc23)c2cccc(-c3cccc(N(c4ccccc4)c4ccc(-c5ccccc5)cc4)c3)c21]

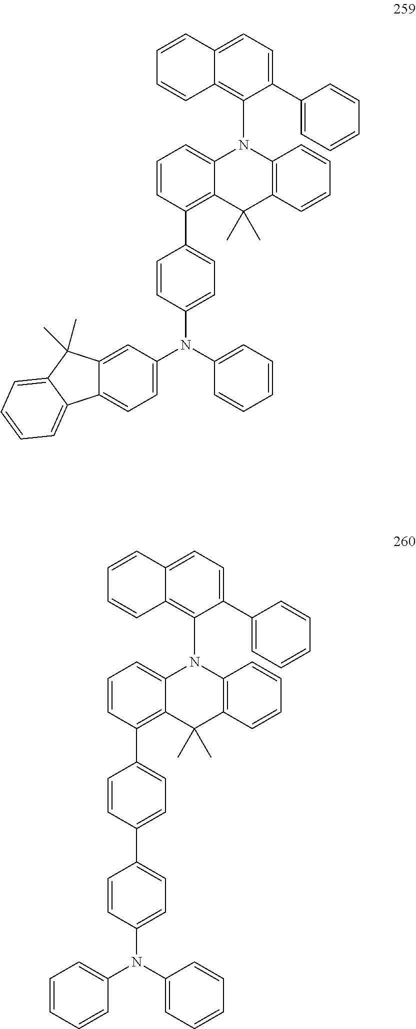 [SMILES: CC1(C)c2ccccc2-c2ccc(N(c3ccccc3)c3ccc(-c4cccc5c4C(C)(C)c4ccccc4N5c4c(-c5ccccc5)ccc5ccccc45)cc3)cc21 ; CC1(C)c2ccccc2N(c2c(-c3ccccc3)ccc3ccccc23)c2cccc(-c3ccc(-c4ccc(N(c5ccccc5)c5ccccc5)cc4)cc3)c21]

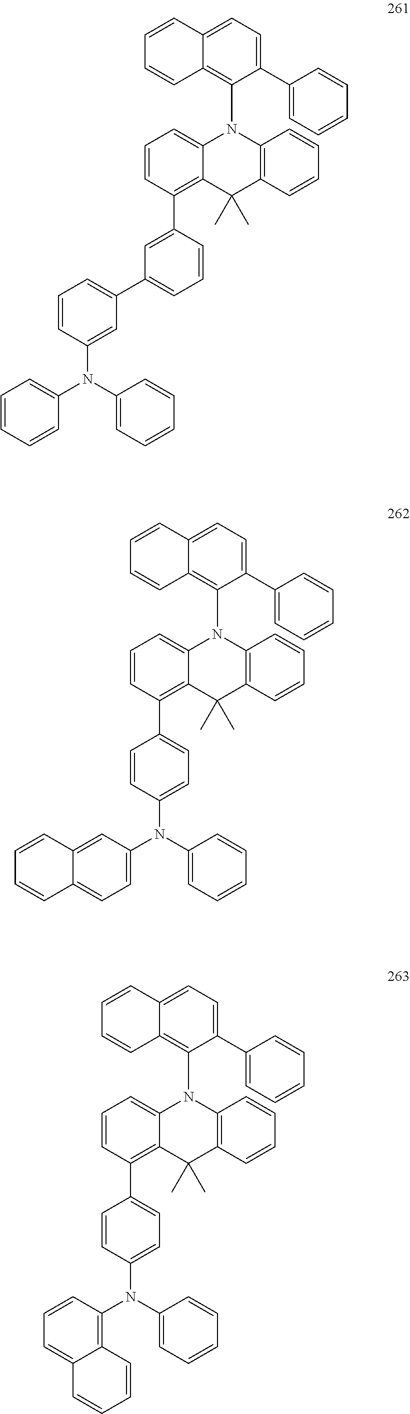 [SMILES: CC1(C)c2ccccc2N(c2c(-c3ccccc3)ccc3ccccc23)c2cccc(-c3cccc(-c4cccc(N(c5ccccc5)c5ccccc5)c4)c3)c21 ; CC1(C)c2ccccc2N(c2c(-c3ccccc3)ccc3ccccc23)c2cccc(-c3ccc(N(c4ccccc4)c4ccc5ccccc5c4)cc3)c21 ; CC1(C)c2ccccc2N(c2c(-c3ccccc3)ccc3ccccc23)c2cccc(-c3ccc(N(c4ccccc4)c4cccc5ccccc45)cc3)c21]

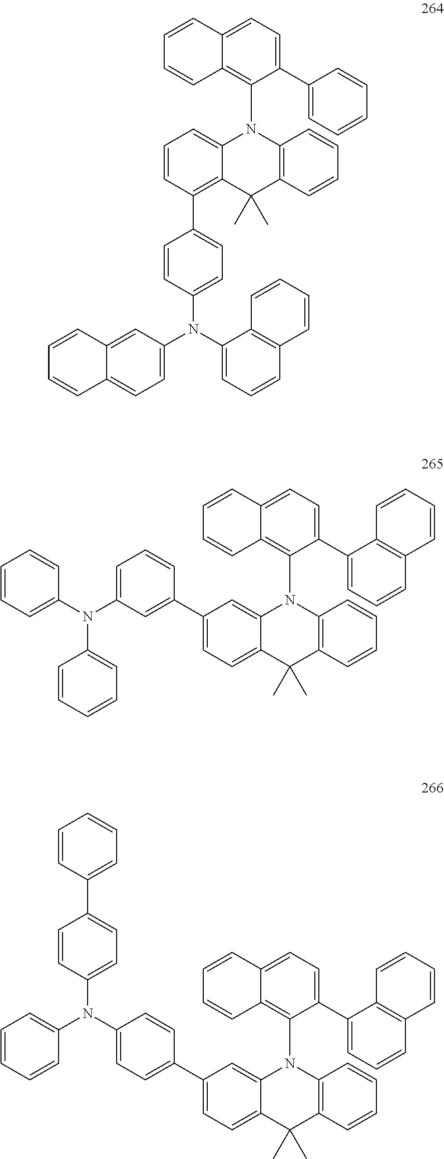 [SMILES: CC1(C)c2ccccc2N(c2c(-c3ccccc3)ccc3ccccc23)c2cccc(-c3ccc(N(c4ccc5ccccc5c4)c4cccc5ccccc45)cc3)c21 ; CC1(C)c2ccccc2N(c2c(-c3cccc4ccccc34)ccc3ccccc23)c2cc(-c3cccc(N(c4ccccc4)c4ccccc4)c3)ccc21 ; CC1(C)c2ccccc2N(c2c(-c3cccc4ccccc34)ccc3ccccc23)c2cc(-c3ccc(N(c4ccccc4)c4ccc(-c5ccccc5)cc4)cc3)ccc21]

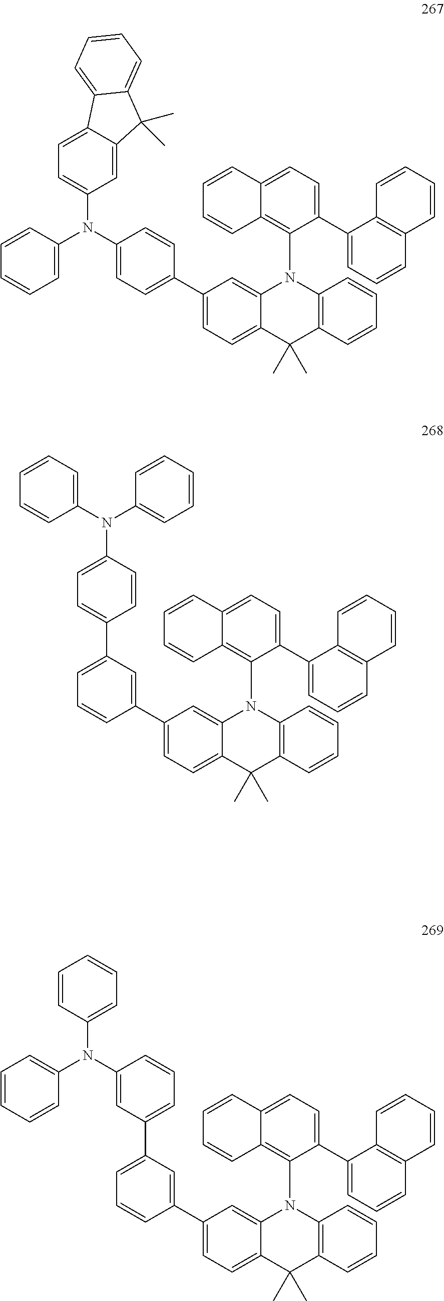 [SMILES: CC1(C)c2ccccc2-c2ccc(N(c3ccccc3)c3ccc(-c4ccc5c(c4)N(c4c(-c6cccc7ccccc67)ccc6ccccc46)c4ccccc4C5(C)C)cc3)cc21 ; CC1(C)c2ccccc2N(c2c(-c3cccc4ccccc34)ccc3ccccc23)c2cc(-c3cccc(-c4ccc(N(c5ccccc5)c5ccccc5)cc4)c3)ccc21 ; CC1(C)c2ccccc2N(c2c(-c3cccc4ccccc34)ccc3ccccc23)c2cc(-c3cccc(-c4cccc(N(c5ccccc5)c5ccccc5)c4)c3)ccc21]

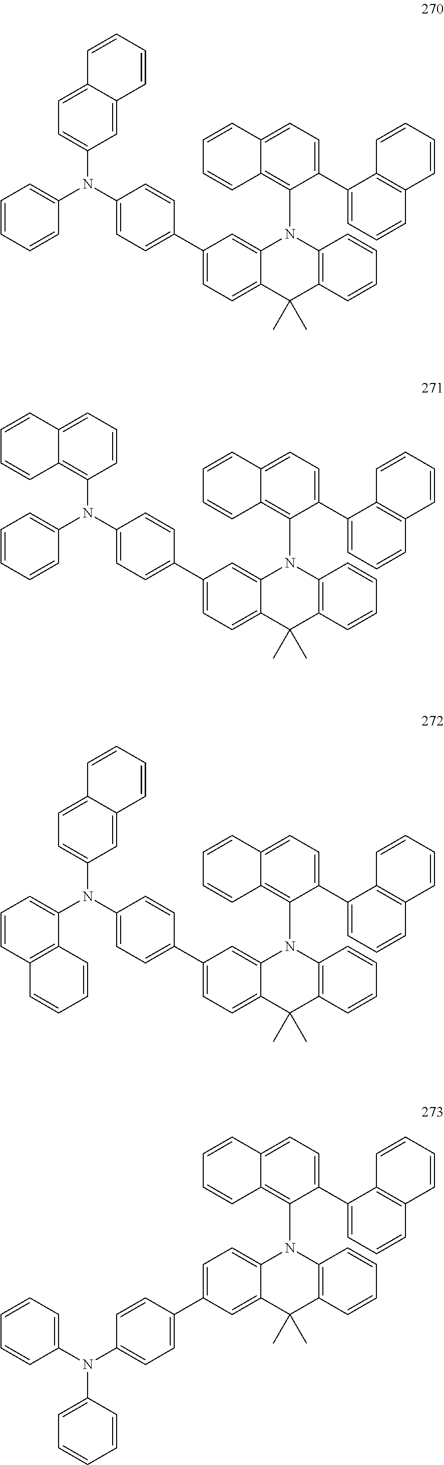 [SMILES: CC1(C)c2ccccc2N(c2c(-c3cccc4ccccc34)ccc3ccccc23)c2cc(-c3ccc(N(c4ccccc4)c4ccc5ccccc5c4)cc3)ccc21 ; CC1(C)c2ccccc2N(c2c(-c3cccc4ccccc34)ccc3ccccc23)c2cc(-c3ccc(N(c4ccccc4)c4cccc5ccccc45)cc3)ccc21 ; CC1(C)c2ccccc2N(c2c(-c3cccc4ccccc34)ccc3ccccc23)c2cc(-c3ccc(N(c4ccc5ccccc5c4)c4cccc5ccccc45)cc3)ccc21 ; CC1(C)c2ccccc2N(c2c(-c3cccc4ccccc34)ccc3ccccc23)c2ccc(-c3ccc(N(c4ccccc4)c4ccccc4)cc3)cc21]

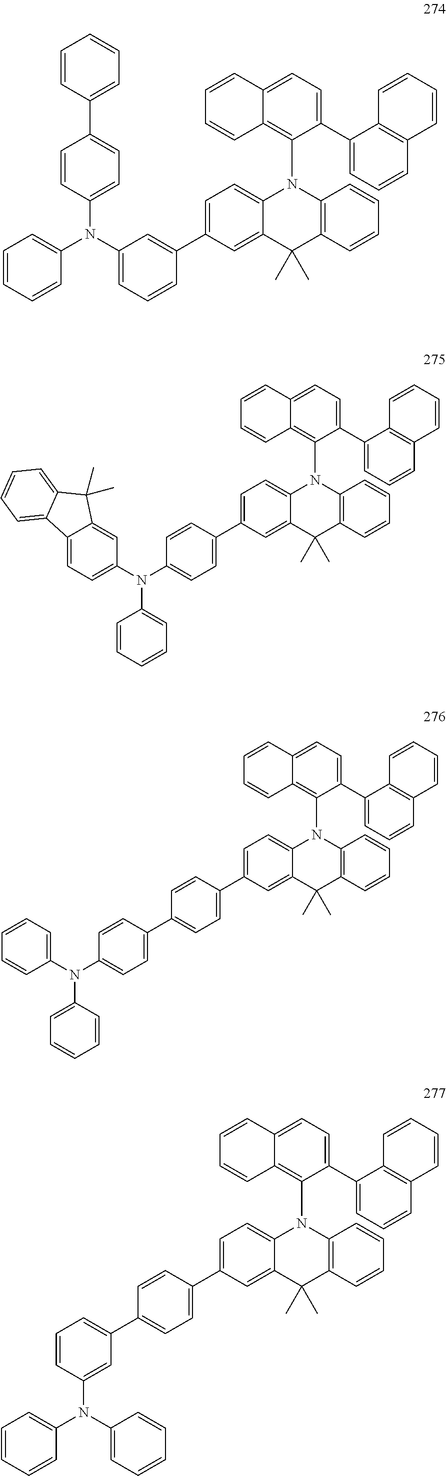 [SMILES: CC1(C)c2ccccc2N(c2c(-c3cccc4ccccc34)ccc3ccccc23)c2ccc(-c3cccc(N(c4ccccc4)c4ccc(-c5ccccc5)cc4)c3)cc21 ; CC1(C)c2ccccc2-c2ccc(N(c3ccccc3)c3ccc(-c4ccc5c(c4)C(C)(C)c4ccccc4N5c4c(-c5cccc6ccccc56)ccc5ccccc45)cc3)cc21 ; CC1(C)c2ccccc2N(c2c(-c3cccc4ccccc34)ccc3ccccc23)c2ccc(-c3ccc(-c4ccc(N(c5ccccc5)c5ccccc5)cc4)cc3)cc21 ; CC1(C)c2ccccc2N(c2c(-c3cccc4ccccc34)ccc3ccccc23)c2ccc(-c3ccc(-c4cccc(N(c5ccccc5)c5ccccc5)c4)cc3)cc21]

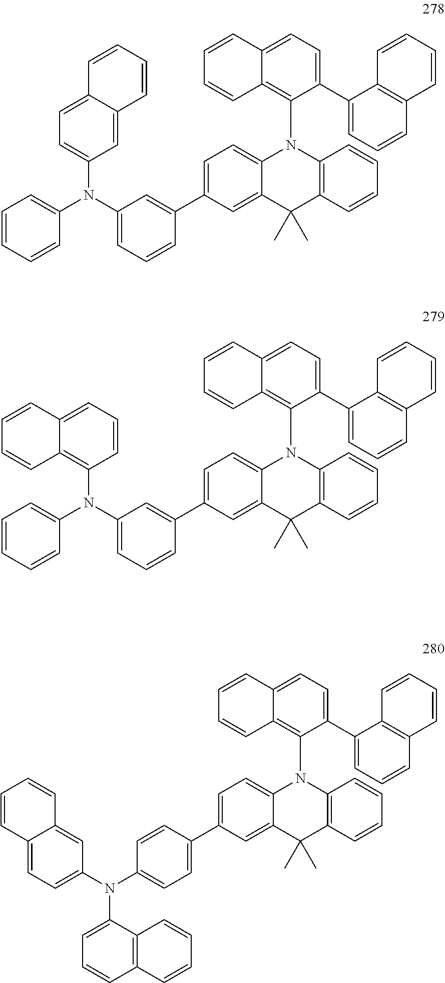 [SMILES: CC1(C)c2ccccc2N(c2c(-c3cccc4ccccc34)ccc3ccccc23)c2ccc(-c3cccc(N(c4ccccc4)c4ccc5ccccc5c4)c3)cc21 ; CC1(C)c2ccccc2N(c2c(-c3cccc4ccccc34)ccc3ccccc23)c2ccc(-c3cccc(N(c4ccccc4)c4cccc5ccccc45)c3)cc21 ; CC1(C)c2ccccc2N(c2c(-c3cccc4ccccc34)ccc3ccccc23)c2ccc(-c3ccc(N(c4ccc5ccccc5c4)c4cccc5ccccc45)cc3)cc21]

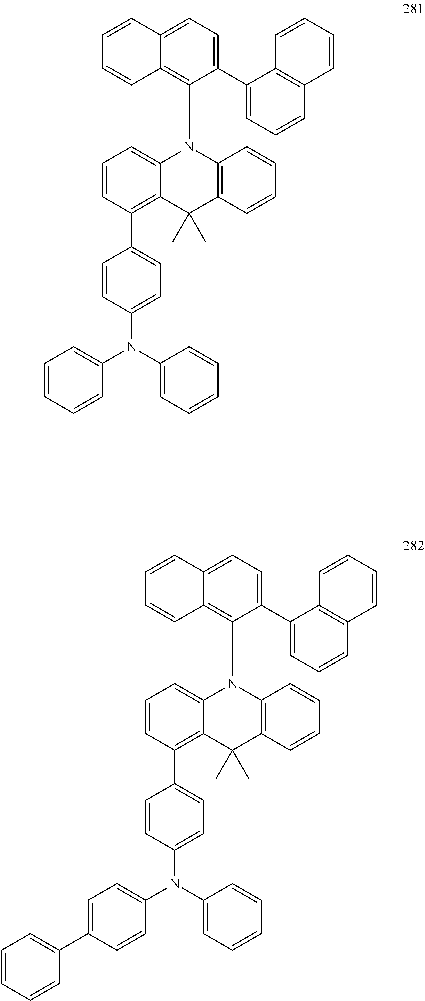 [SMILES: CC1(C)c2ccccc2N(c2c(-c3cccc4ccccc34)ccc3ccccc23)c2cccc(-c3ccc(N(c4ccccc4)c4ccccc4)cc3)c21 ; CC1(C)c2ccccc2N(c2c(-c3cccc4ccccc34)ccc3ccccc23)c2cccc(-c3ccc(N(c4ccccc4)c4ccc(-c5ccccc5)cc4)cc3)c21]

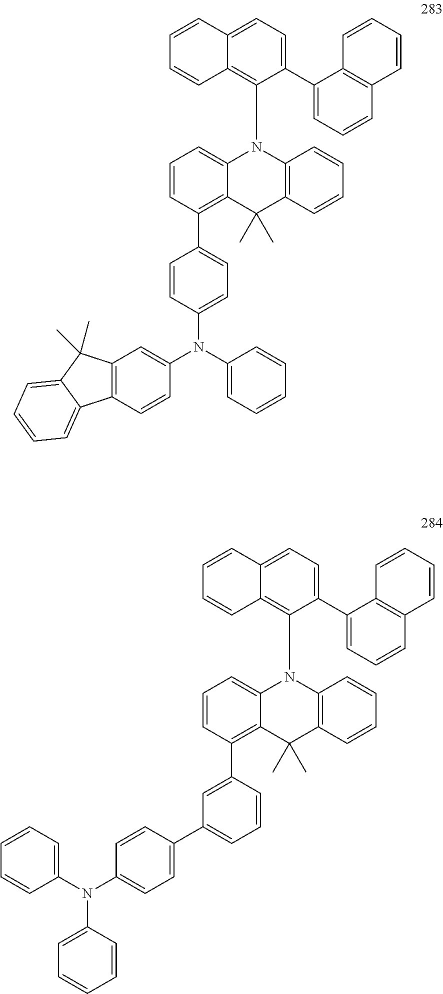 [SMILES: CC1(C)c2ccccc2-c2ccc(N(c3ccccc3)c3ccc(-c4cccc5c4C(C)(C)c4ccccc4N5c4c(-c5cccc6ccccc56)ccc5ccccc45)cc3)cc21 ; CC1(C)c2ccccc2N(c2c(-c3cccc4ccccc34)ccc3ccccc23)c2cccc(-c3cccc(-c4ccc(N(c5ccccc5)c5ccccc5)cc4)c3)c21]

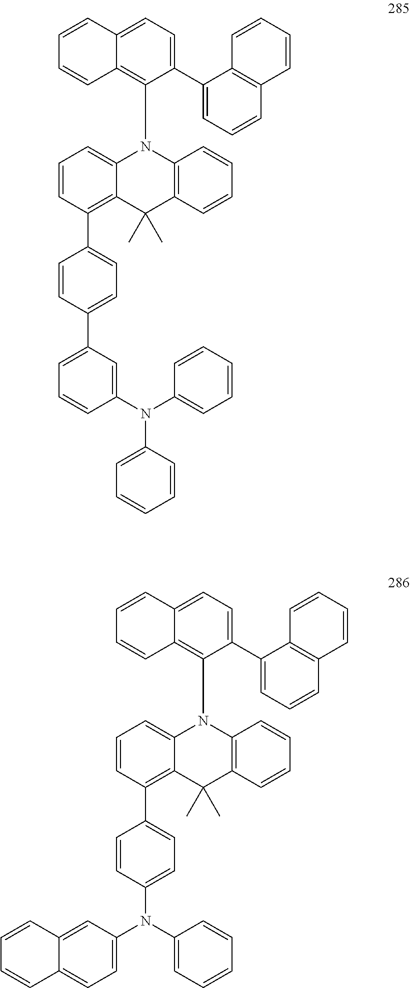 [SMILES: CC1(C)c2ccccc2N(c2c(-c3cccc4ccccc34)ccc3ccccc23)c2cccc(-c3ccc(-c4cccc(N(c5ccccc5)c5ccccc5)c4)cc3)c21 ; CC1(C)c2ccccc2N(c2c(-c3cccc4ccccc34)ccc3ccccc23)c2cccc(-c3ccc(N(c4ccccc4)c4ccc5ccccc5c4)cc3)c21]

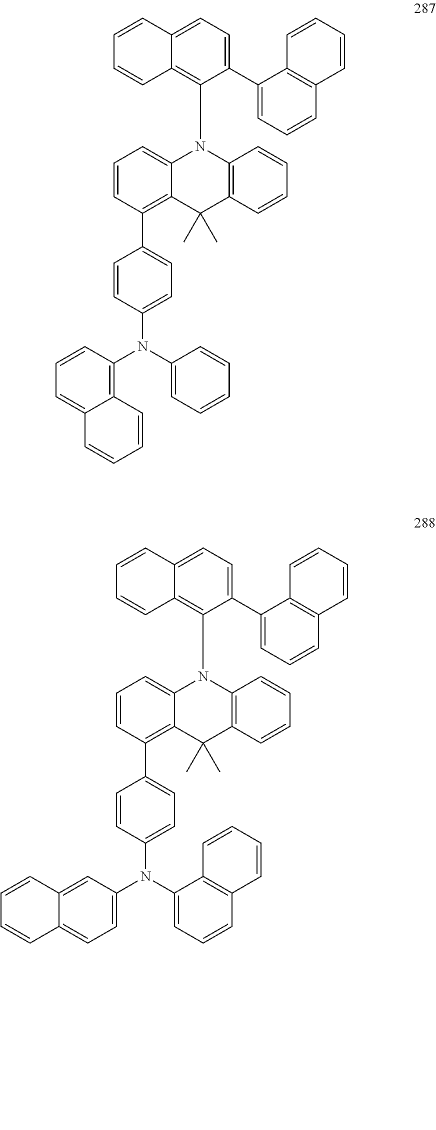 [SMILES: CC1(C)c2ccccc2N(c2c(-c3cccc4ccccc34)ccc3ccccc23)c2cccc(-c3ccc(N(c4ccccc4)c4cccc5ccccc45)cc3)c21 ; CC1(C)c2ccccc2N(c2c(-c3cccc4ccccc34)ccc3ccccc23)c2cccc(-c3ccc(N(c4ccc5ccccc5c4)c4cccc5ccccc45)cc3)c21]

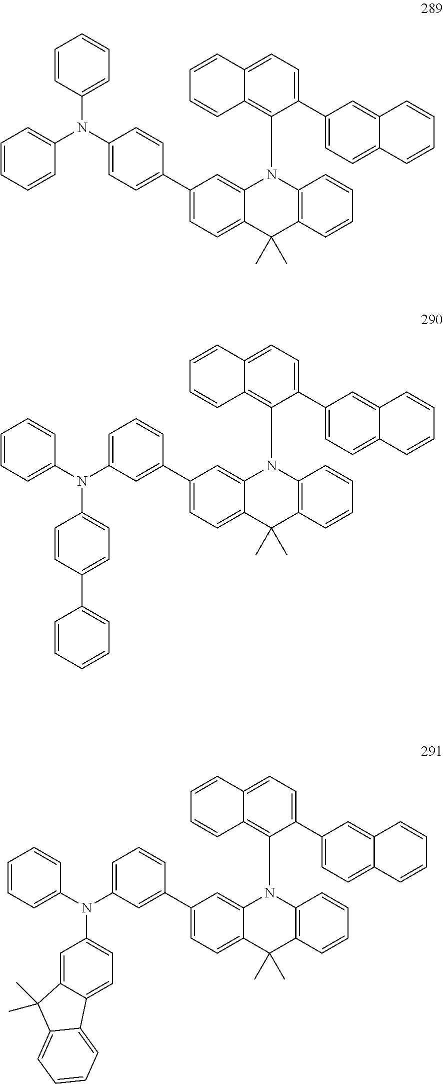 [SMILES: CC1(C)c2ccccc2N(c2c(-c3ccc4ccccc4c3)ccc3ccccc23)c2cc(-c3ccc(N(c4ccccc4)c4ccccc4)cc3)ccc21 ; CC1(C)c2ccccc2N(c2c(-c3ccc4ccccc4c3)ccc3ccccc23)c2cc(-c3cccc(N(c4ccccc4)c4ccc(-c5ccccc5)cc4)c3)ccc21 ; CC1(C)c2ccccc2-c2ccc(N(c3ccccc3)c3cccc(-c4ccc5c(c4)N(c4c(-c6ccc7ccccc7c6)ccc6ccccc46)c4ccccc4C5(C)C)c3)cc21]

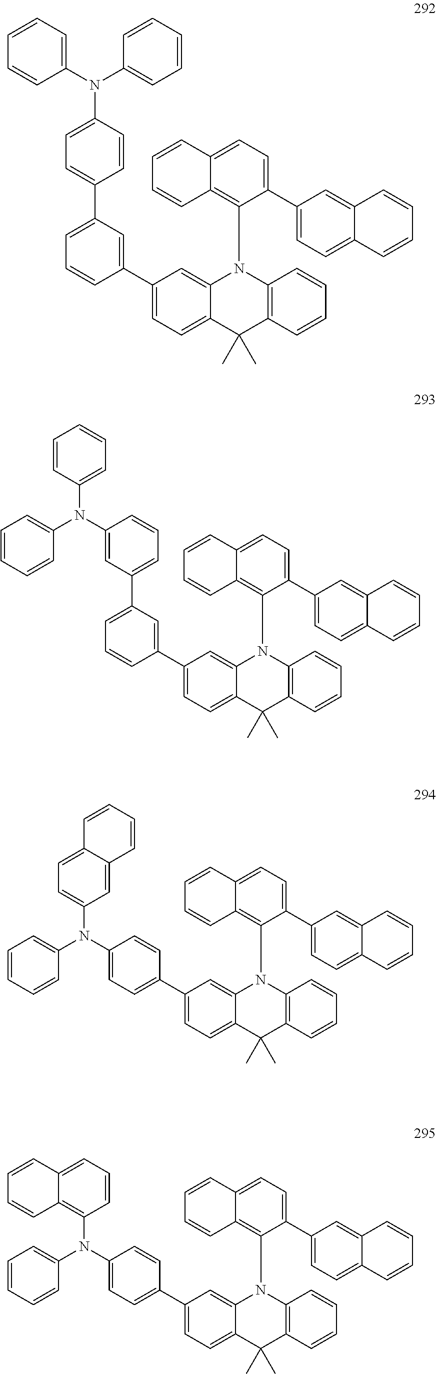 [SMILES: CC1(C)c2ccccc2N(c2c(-c3ccc4ccccc4c3)ccc3ccccc23)c2cc(-c3cccc(-c4ccc(N(c5ccccc5)c5ccccc5)cc4)c3)ccc21 ; CC1(C)c2ccccc2N(c2c(-c3ccc4ccccc4c3)ccc3ccccc23)c2cc(-c3cccc(-c4cccc(N(c5ccccc5)c5ccccc5)c4)c3)ccc21 ; CC1(C)c2ccccc2N(c2c(-c3ccc4ccccc4c3)ccc3ccccc23)c2cc(-c3ccc(N(c4ccccc4)c4ccc5ccccc5c4)cc3)ccc21 ; CC1(C)c2ccccc2N(c2c(-c3ccc4ccccc4c3)ccc3ccccc23)c2cc(-c3ccc(N(c4ccccc4)c4cccc5ccccc45)cc3)ccc21]

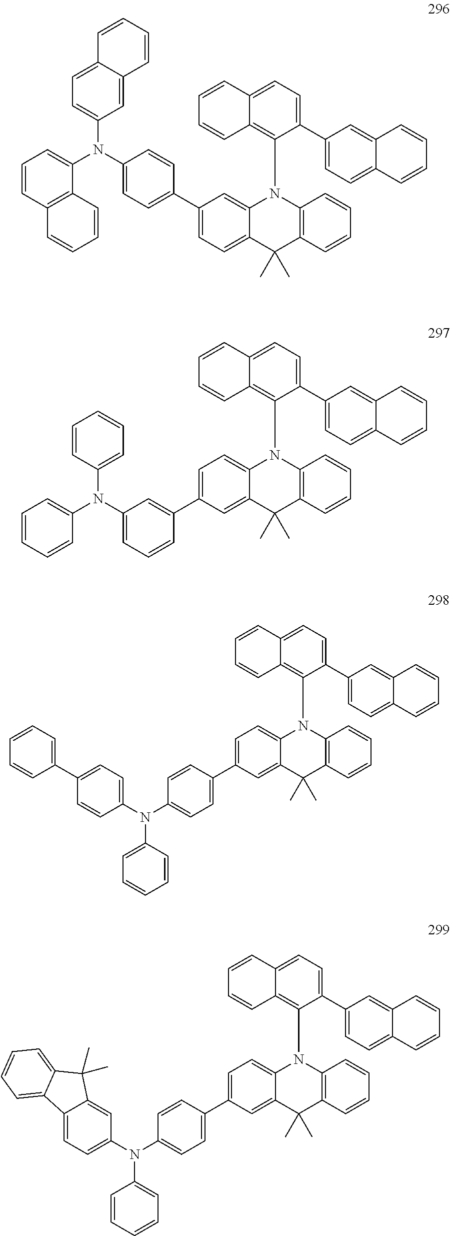 [SMILES: CC1(C)c2ccccc2N(c2c(-c3ccc4ccccc4c3)ccc3ccccc23)c2cc(-c3ccc(N(c4ccc5ccccc5c4)c4cccc5ccccc45)cc3)ccc21 ; CC1(C)c2ccccc2N(c2c(-c3ccc4ccccc4c3)ccc3ccccc23)c2ccc(-c3cccc(N(c4ccccc4)c4ccccc4)c3)cc21 ; CC1(C)c2ccccc2N(c2c(-c3ccc4ccccc4c3)ccc3ccccc23)c2ccc(-c3ccc(N(c4ccccc4)c4ccc(-c5ccccc5)cc4)cc3)cc21 ; CC1(C)c2ccccc2-c2ccc(N(c3ccccc3)c3ccc(-c4ccc5c(c4)C(C)(C)c4ccccc4N5c4c(-c5ccc6ccccc6c5)ccc5ccccc45)cc3)cc21]

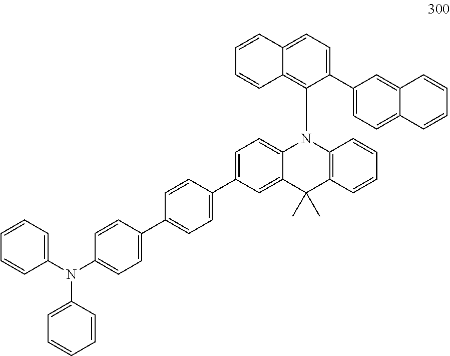 [SMILES: CC1(C)c2ccccc2N(c2c(-c3ccc4ccccc4c3)ccc3ccccc23)c2ccc(-c3ccc(-c4ccc(N(c5ccccc5)c5ccccc5)cc4)cc3)cc21]

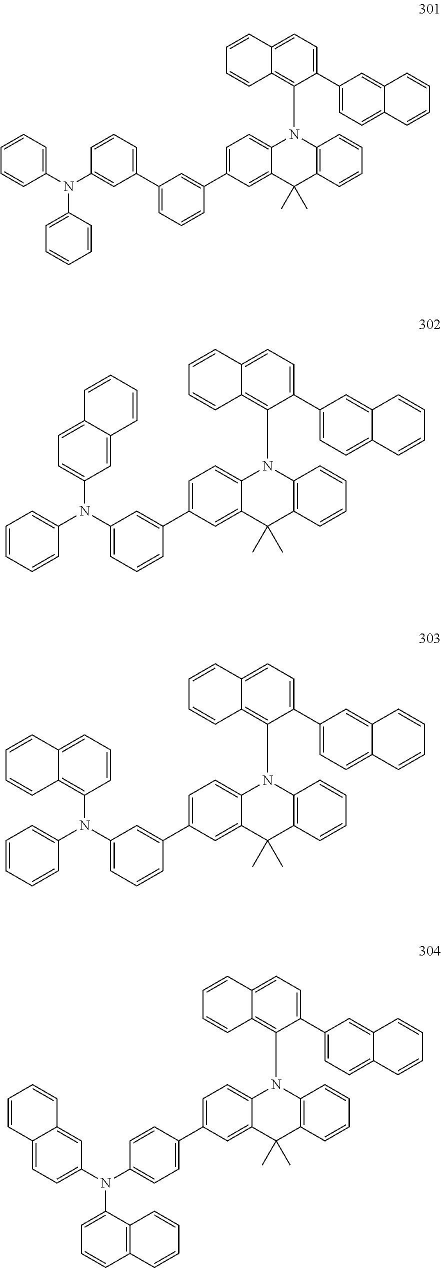 [SMILES: CC1(C)c2ccccc2N(c2c(-c3ccc4ccccc4c3)ccc3ccccc23)c2ccc(-c3cccc(-c4cccc(N(c5ccccc5)c5ccccc5)c4)c3)cc21 ; CC1(C)c2ccccc2N(c2c(-c3ccc4ccccc4c3)ccc3ccccc23)c2ccc(-c3cccc(N(c4ccccc4)c4ccc5ccccc5c4)c3)cc21 ; CC1(C)c2ccccc2N(c2c(-c3ccc4ccccc4c3)ccc3ccccc23)c2ccc(-c3cccc(N(c4ccccc4)c4cccc5ccccc45)c3)cc21 ; CC1(C)c2ccccc2N(c2c(-c3ccc4ccccc4c3)ccc3ccccc23)c2ccc(-c3ccc(N(c4ccc5ccccc5c4)c4cccc5ccccc45)cc3)cc21]

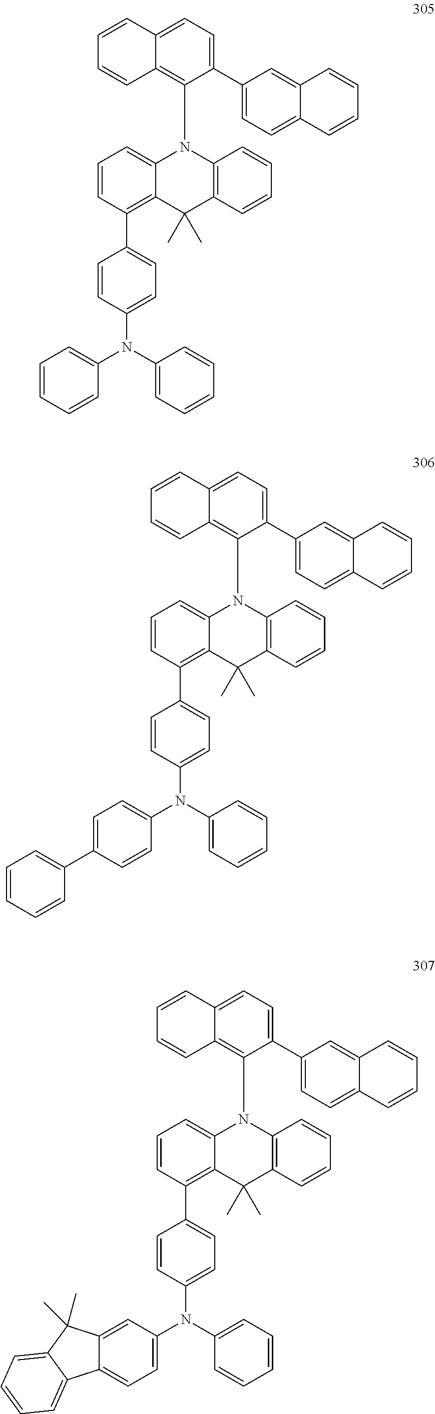 [SMILES: CC1(C)c2ccccc2N(c2c(-c3ccc4ccccc4c3)ccc3ccccc23)c2cccc(-c3ccc(N(c4ccccc4)c4ccccc4)cc3)c21 ; CC1(C)c2ccccc2N(c2c(-c3ccc4ccccc4c3)ccc3ccccc23)c2cccc(-c3ccc(N(c4ccccc4)c4ccc(-c5ccccc5)cc4)cc3)c21 ; CC1(C)c2ccccc2-c2ccc(N(c3ccccc3)c3ccc(-c4cccc5c4C(C)(C)c4ccccc4N5c4c(-c5ccc6ccccc6c5)ccc5ccccc45)cc3)cc21]

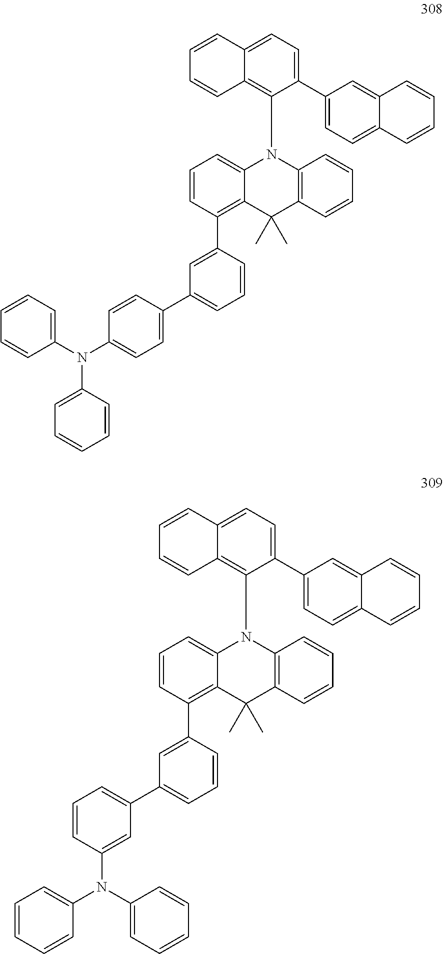 [SMILES: CC1(C)c2ccccc2N(c2c(-c3ccc4ccccc4c3)ccc3ccccc23)c2cccc(-c3cccc(-c4ccc(N(c5ccccc5)c5ccccc5)cc4)c3)c21 ; CC1(C)c2ccccc2N(c2c(-c3ccc4ccccc4c3)ccc3ccccc23)c2cccc(-c3cccc(-c4cccc(N(c5ccccc5)c5ccccc5)c4)c3)c21]

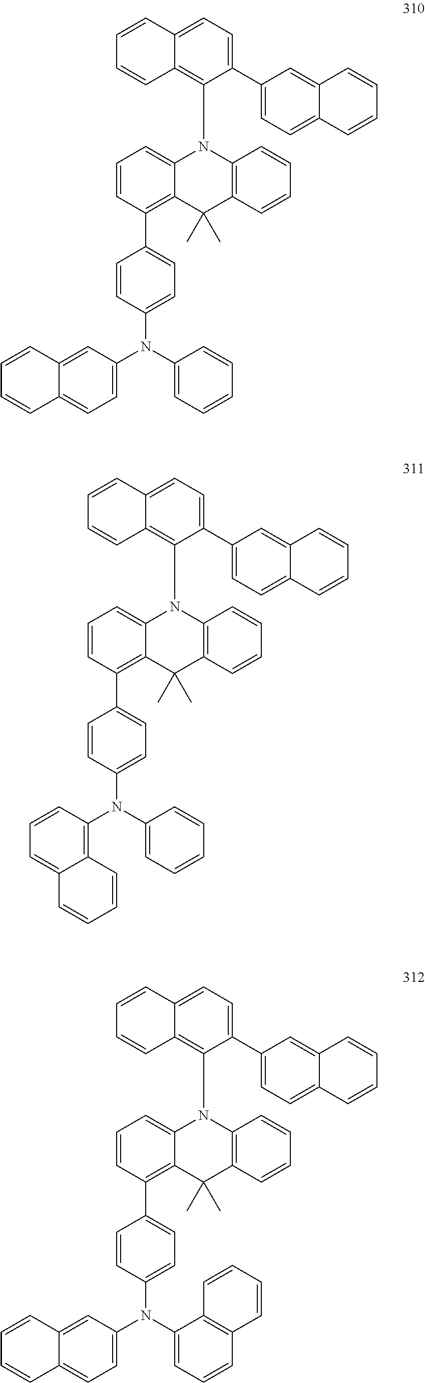 [SMILES: CC1(C)c2ccccc2N(c2c(-c3ccc4ccccc4c3)ccc3ccccc23)c2cccc(-c3ccc(N(c4ccccc4)c4ccc5ccccc5c4)cc3)c21 ; CC1(C)c2ccccc2N(c2c(-c3ccc4ccccc4c3)ccc3ccccc23)c2cccc(-c3ccc(N(c4ccccc4)c4cccc5ccccc45)cc3)c21 ; CC1(C)c2ccccc2N(c2c(-c3ccc4ccccc4c3)ccc3ccccc23)c2cccc(-c3ccc(N(c4ccc5ccccc5c4)c4cccc5ccccc45)cc3)c21]

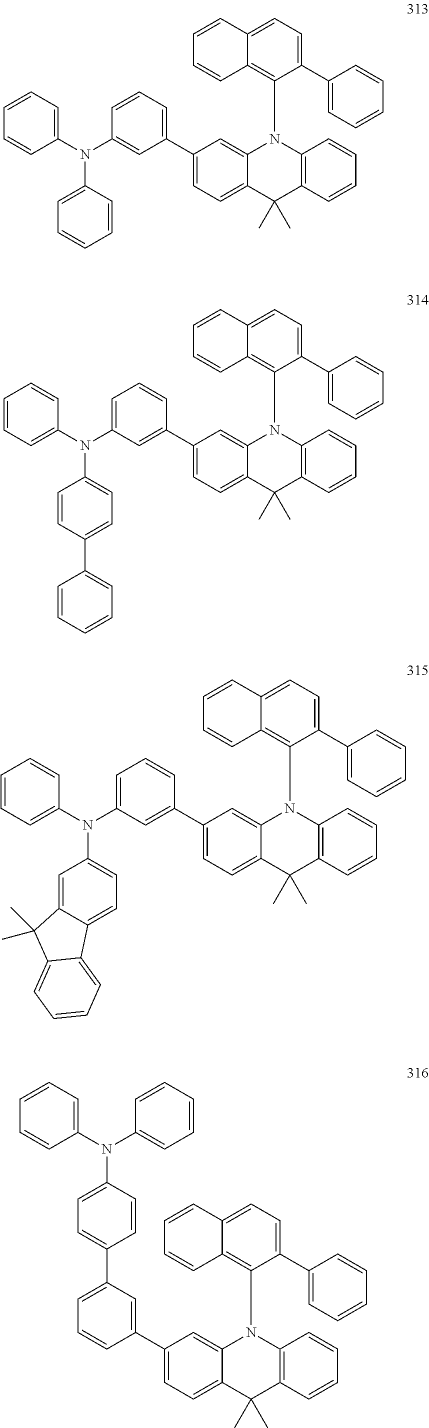 [SMILES: CC1(C)c2ccccc2N(c2c(-c3ccccc3)ccc3ccccc23)c2cc(-c3cccc(N(c4ccccc4)c4ccccc4)c3)ccc21 ; CC1(C)c2ccccc2N(c2c(-c3ccccc3)ccc3ccccc23)c2cc(-c3cccc(N(c4ccccc4)c4ccc(-c5ccccc5)cc4)c3)ccc21 ; CC1(C)c2ccccc2-c2ccc(N(c3ccccc3)c3cccc(-c4ccc5c(c4)N(c4c(-c6ccccc6)ccc6ccccc46)c4ccccc4C5(C)C)c3)cc21 ; CC1(C)c2ccccc2N(c2c(-c3ccccc3)ccc3ccccc23)c2cc(-c3cccc(-c4ccc(N(c5ccccc5)c5ccccc5)cc4)c3)ccc21]

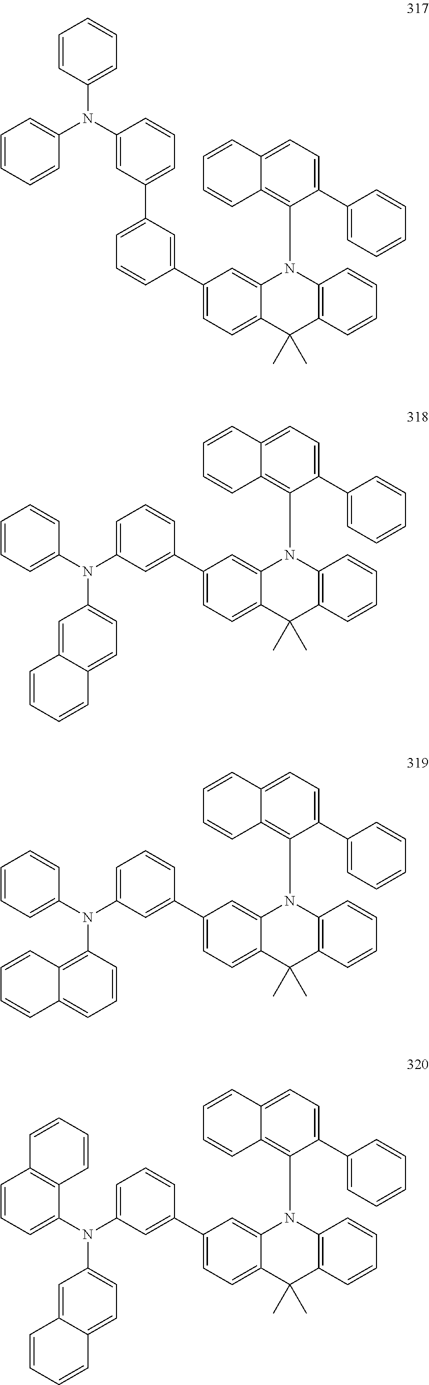 [SMILES: CC1(C)c2ccccc2N(c2c(-c3ccccc3)ccc3ccccc23)c2cc(-c3cccc(-c4cccc(N(c5ccccc5)c5ccccc5)c4)c3)ccc21 ; CC1(C)c2ccccc2N(c2c(-c3ccccc3)ccc3ccccc23)c2cc(-c3cccc(N(c4ccccc4)c4ccc5ccccc5c4)c3)ccc21 ; CC1(C)c2ccccc2N(c2c(-c3ccccc3)ccc3ccccc23)c2cc(-c3cccc(N(c4ccccc4)c4cccc5ccccc45)c3)ccc21 ; CC1(C)c2ccccc2N(c2c(-c3ccccc3)ccc3ccccc23)c2cc(-c3cccc(N(c4ccc5ccccc5c4)c4cccc5ccccc45)c3)ccc21]

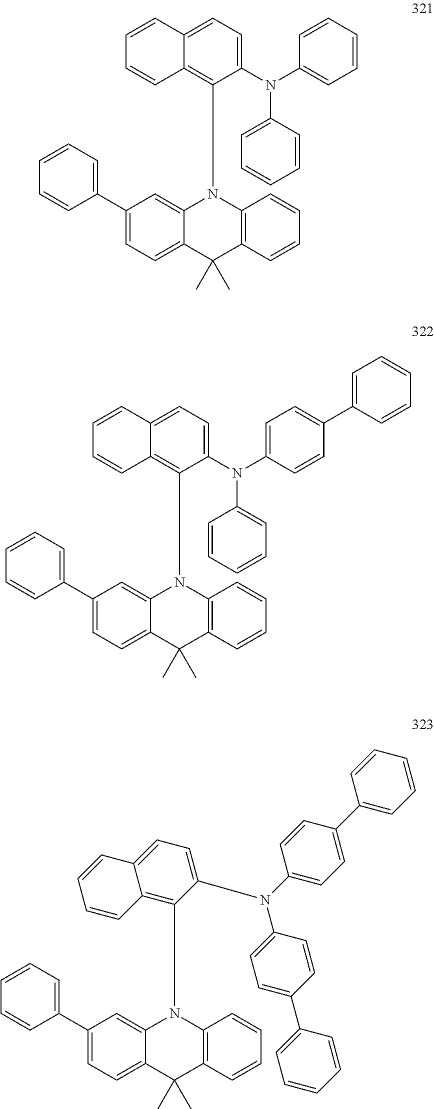 [SMILES: CC1(C)c2ccccc2N(c2c(N(c3ccccc3)c3ccccc3)ccc3ccccc23)c2cc(-c3ccccc3)ccc21 ; CC1(C)c2ccccc2N(c2c(N(c3ccccc3)c3ccc(-c4ccccc4)cc3)ccc3ccccc23)c2cc(-c3ccccc3)ccc21 ; CC1(C)c2ccccc2N(c2c(N(c3ccc(-c4ccccc4)cc3)c3ccc(-c4ccccc4)cc3)ccc3ccccc23)c2cc(-c3ccccc3)ccc21]

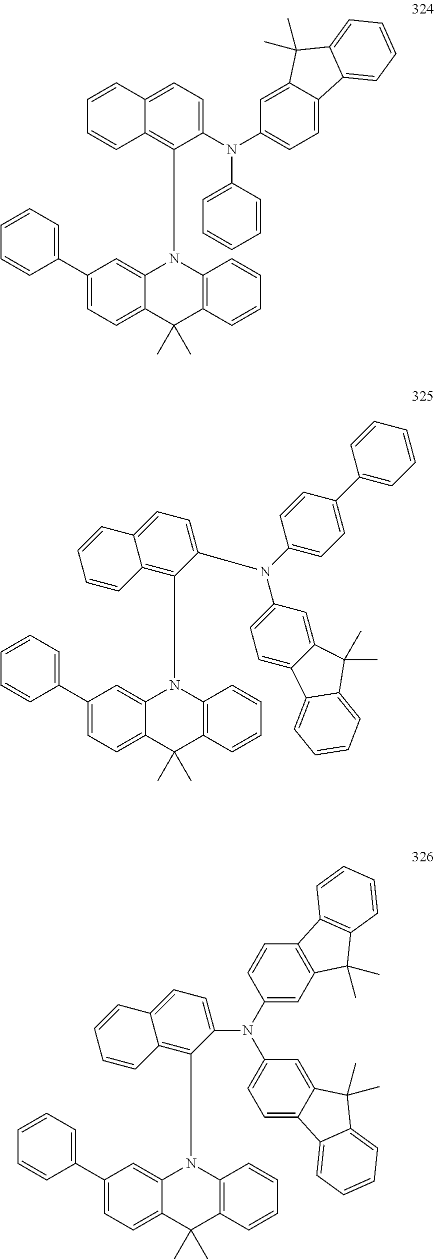 [SMILES: CC1(C)c2ccccc2-c2ccc(N(c3ccccc3)c3ccc4ccccc4c3N3c4ccccc4C(C)(C)c4ccc(-c5ccccc5)cc43)cc21 ; CC1(C)c2ccccc2-c2ccc(N(c3ccc(-c4ccccc4)cc3)c3ccc4ccccc4c3N3c4ccccc4C(C)(C)c4ccc(-c5ccccc5)cc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4ccc(-c5ccccc5)cc43)cc21]

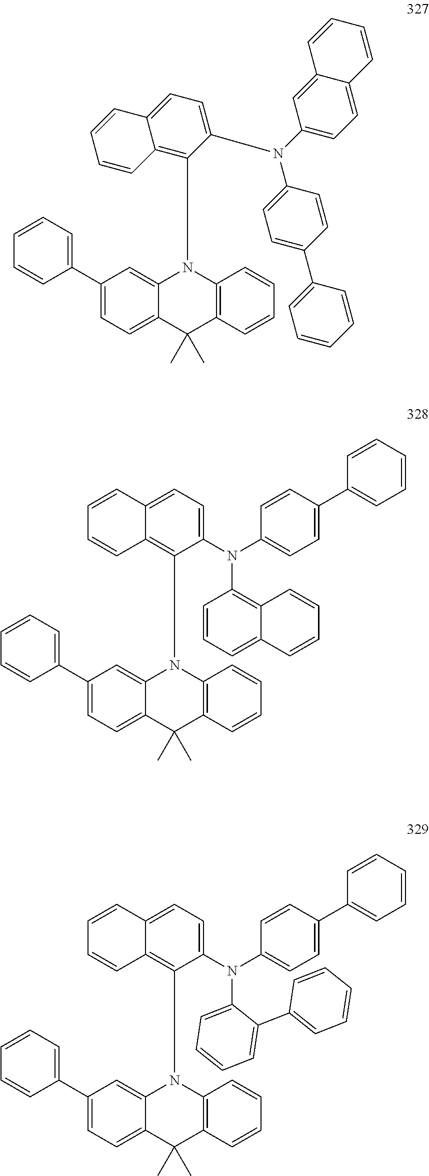 [SMILES: CC1(C)c2ccccc2N(c2c(N(c3ccc(-c4ccccc4)cc3)c3ccc4ccccc4c3)ccc3ccccc23)c2cc(-c3ccccc3)ccc21 ; CC1(C)c2ccccc2N(c2c(N(c3ccc(-c4ccccc4)cc3)c3cccc4ccccc34)ccc3ccccc23)c2cc(-c3ccccc3)ccc21 ; CC1(C)c2ccccc2N(c2c(N(c3ccc(-c4ccccc4)cc3)c3ccccc3-c3ccccc3)ccc3ccccc23)c2cc(-c3ccccc3)ccc21]

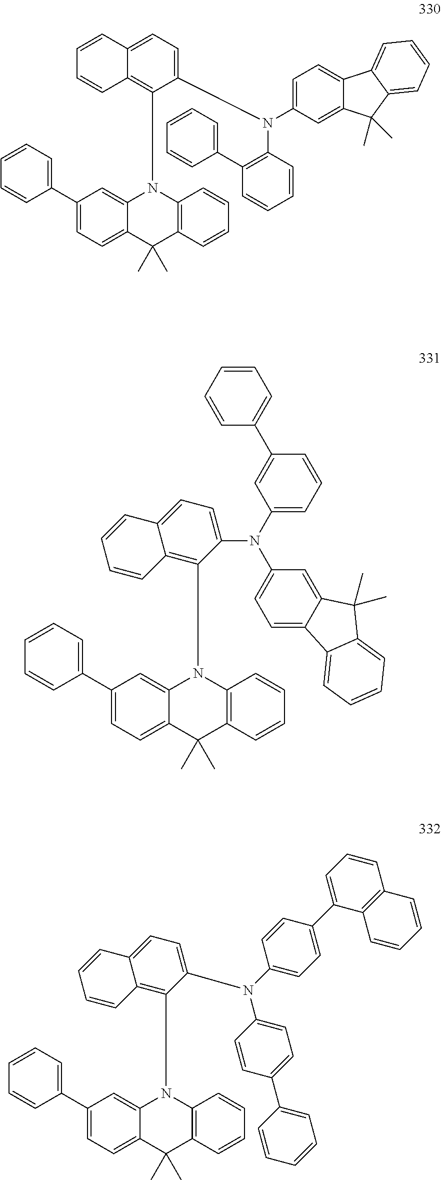 [SMILES: CC1(C)c2ccccc2-c2ccc(N(c3ccccc3-c3ccccc3)c3ccc4ccccc4c3N3c4ccccc4C(C)(C)c4ccc(-c5ccccc5)cc43)cc21 ; CC1(C)c2ccccc2-c2ccc(N(c3cccc(-c4ccccc4)c3)c3ccc4ccccc4c3N3c4ccccc4C(C)(C)c4ccc(-c5ccccc5)cc43)cc21 ; CC1(C)c2ccccc2N(c2c(N(c3ccc(-c4ccccc4)cc3)c3ccc(-c4cccc5ccccc45)cc3)ccc3ccccc23)c2cc(-c3ccccc3)ccc21]

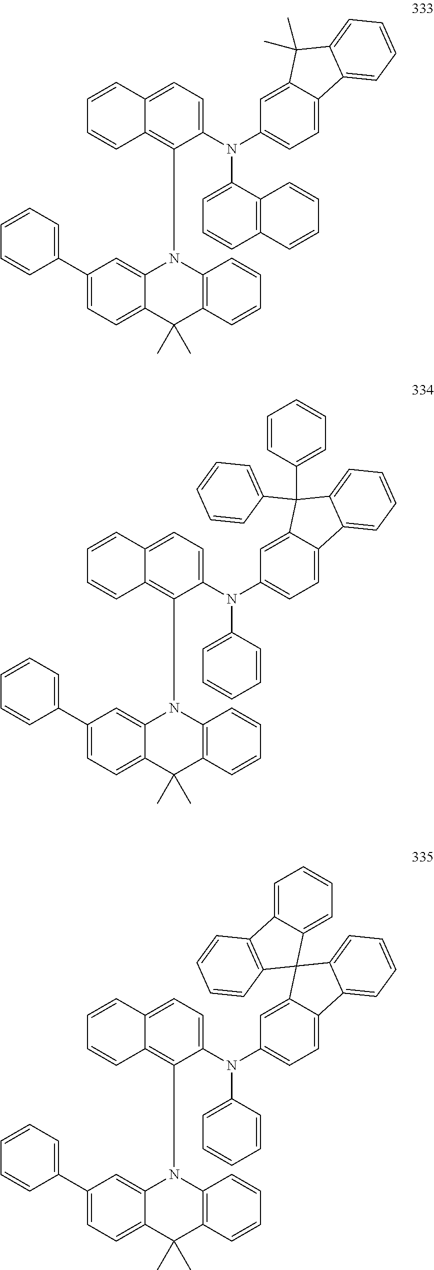 [SMILES: CC1(C)c2ccccc2-c2ccc(N(c3ccc4ccccc4c3N3c4ccccc4C(C)(C)c4ccc(-c5ccccc5)cc43)c3cccc4ccccc34)cc21 ; CC1(C)c2ccccc2N(c2c(N(c3ccccc3)c3ccc4c(c3)C(c3ccccc3)(c3ccccc3)c3ccccc3-4)ccc3ccccc23)c2cc(-c3ccccc3)ccc21 ; CC1(C)c2ccccc2N(c2c(N(c3ccccc3)c3ccc4c(c3)C3(c5ccccc5-c5ccccc53)c3ccccc3-4)ccc3ccccc23)c2cc(-c3ccccc3)ccc21]

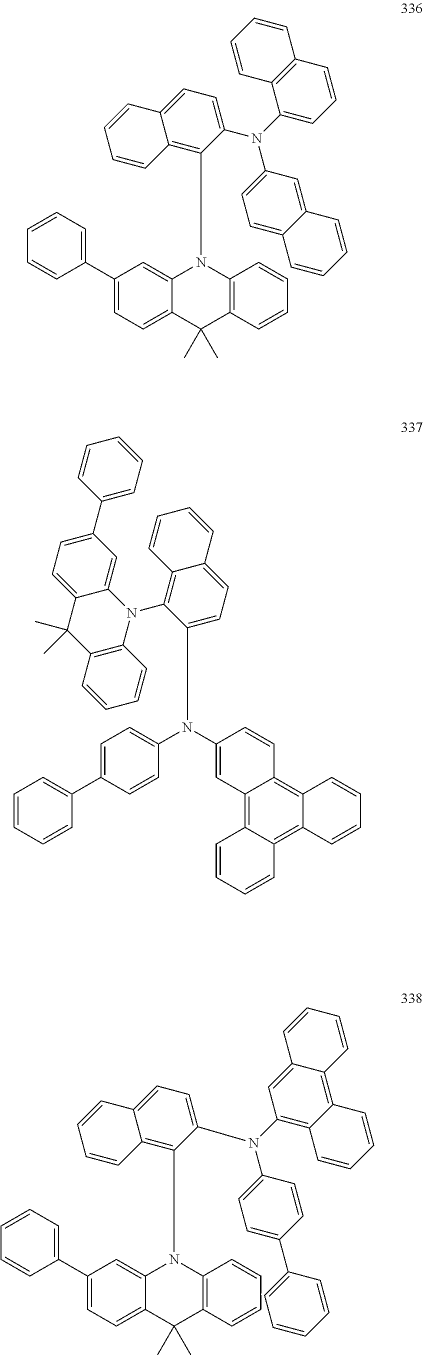 [SMILES: CC1(C)c2ccccc2N(c2c(N(c3ccc4ccccc4c3)c3cccc4ccccc34)ccc3ccccc23)c2cc(-c3ccccc3)ccc21 ; CC1(C)c2ccccc2N(c2c(N(c3ccc(-c4ccccc4)cc3)c3ccc4c5ccccc5c5ccccc5c4c3)ccc3ccccc23)c2cc(-c3ccccc3)ccc21 ; CC1(C)c2ccccc2N(c2c(N(c3ccc(-c4ccccc4)cc3)c3cc4ccccc4c4ccccc34)ccc3ccccc23)c2cc(-c3ccccc3)ccc21]

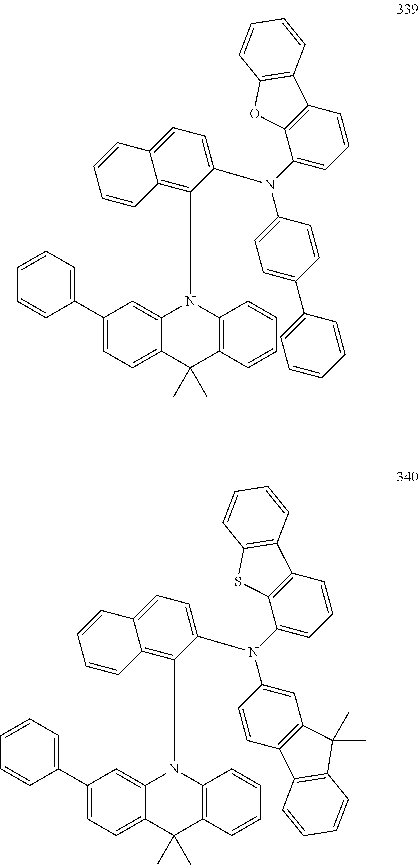 [SMILES: CC1(C)c2ccccc2N(c2c(N(c3ccc(-c4ccccc4)cc3)c3cccc4c3oc3ccccc34)ccc3ccccc23)c2cc(-c3ccccc3)ccc21 ; CC1(C)c2ccccc2-c2ccc(N(c3ccc4ccccc4c3N3c4ccccc4C(C)(C)c4ccc(-c5ccccc5)cc43)c3cccc4c3sc3ccccc34)cc21]

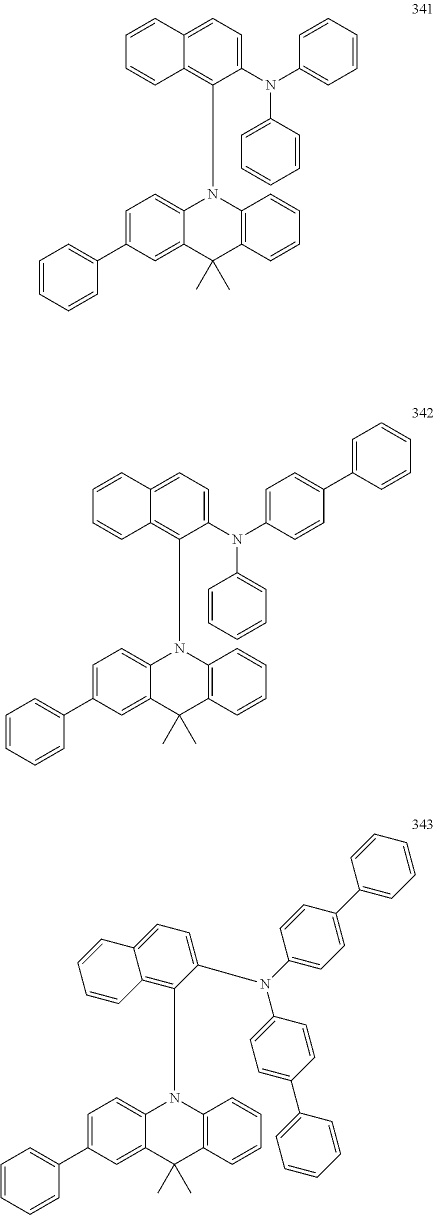 [SMILES: CC1(C)c2ccccc2N(c2c(N(c3ccccc3)c3ccccc3)ccc3ccccc23)c2ccc(-c3ccccc3)cc21 ; CC1(C)c2ccccc2N(c2c(N(c3ccccc3)c3ccc(-c4ccccc4)cc3)ccc3ccccc23)c2ccc(-c3ccccc3)cc21 ; CC1(C)c2ccccc2N(c2c(N(c3ccc(-c4ccccc4)cc3)c3ccc(-c4ccccc4)cc3)ccc3ccccc23)c2ccc(-c3ccccc3)cc21]

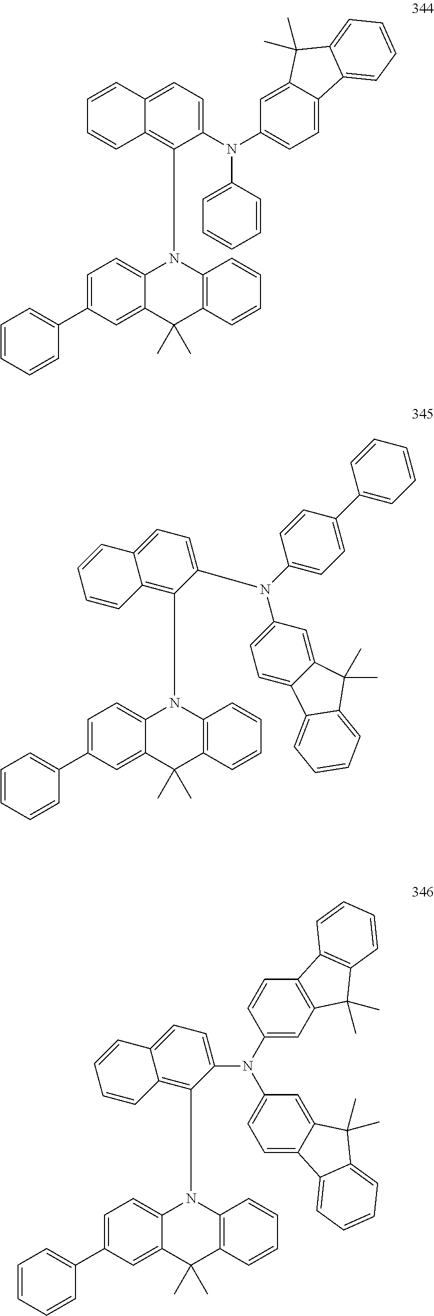 [SMILES: CC1(C)c2ccccc2-c2ccc(N(c3ccccc3)c3ccc4ccccc4c3N3c4ccccc4C(C)(C)c4cc(-c5ccccc5)ccc43)cc21 ; CC1(C)c2ccccc2-c2ccc(N(c3ccc(-c4ccccc4)cc3)c3ccc4ccccc4c3N3c4ccccc4C(C)(C)c4cc(-c5ccccc5)ccc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4cc(-c5ccccc5)ccc43)cc21]

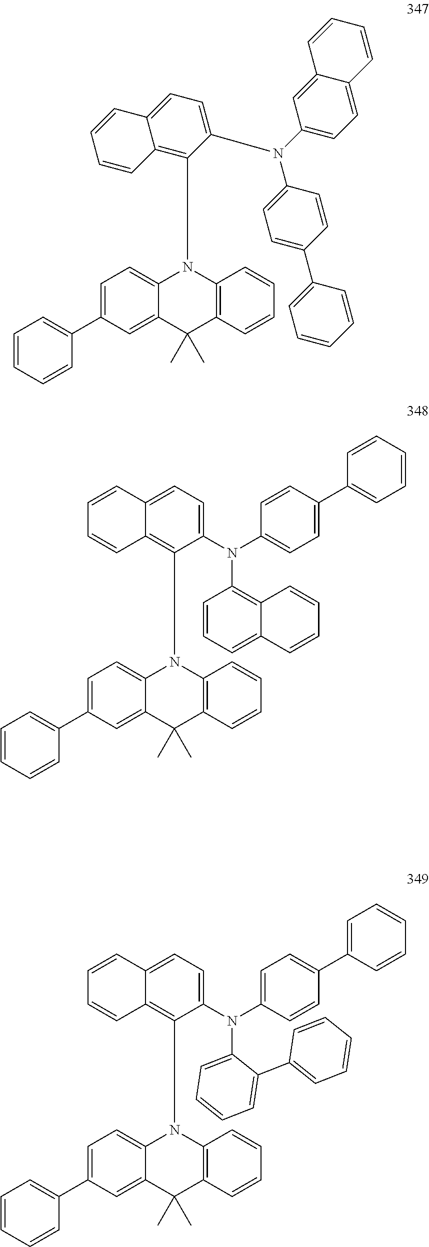 [SMILES: CC1(C)c2ccccc2N(c2c(N(c3ccc(-c4ccccc4)cc3)c3ccc4ccccc4c3)ccc3ccccc23)c2ccc(-c3ccccc3)cc21 ; CC1(C)c2ccccc2N(c2c(N(c3ccc(-c4ccccc4)cc3)c3cccc4ccccc34)ccc3ccccc23)c2ccc(-c3ccccc3)cc21 ; CC1(C)c2ccccc2N(c2c(N(c3ccc(-c4ccccc4)cc3)c3ccccc3-c3ccccc3)ccc3ccccc23)c2ccc(-c3ccccc3)cc21]

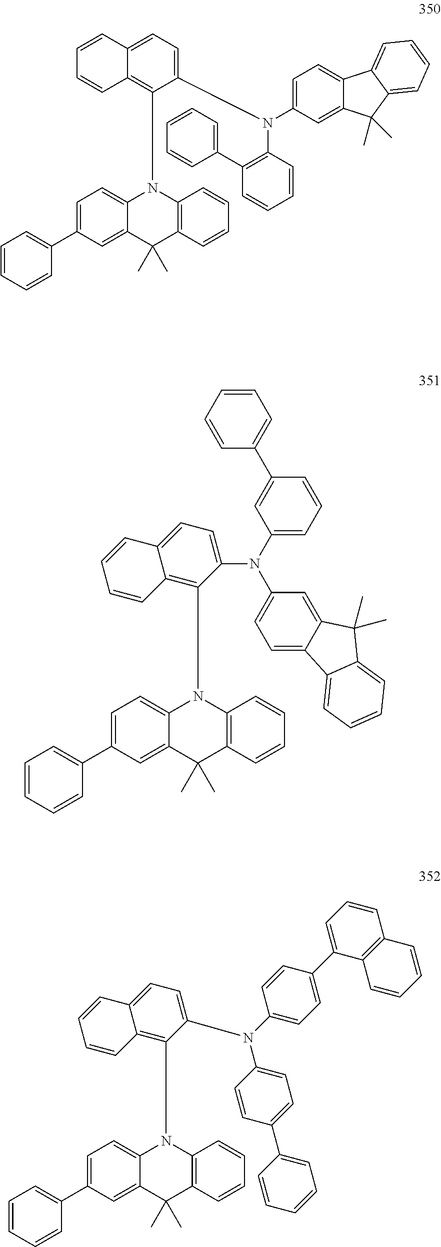 [SMILES: CC1(C)c2ccccc2-c2ccc(N(c3ccccc3-c3ccccc3)c3ccc4ccccc4c3N3c4ccccc4C(C)(C)c4cc(-c5ccccc5)ccc43)cc21 ; CC1(C)c2ccccc2-c2ccc(N(c3cccc(-c4ccccc4)c3)c3ccc4ccccc4c3N3c4ccccc4C(C)(C)c4cc(-c5ccccc5)ccc43)cc21 ; CC1(C)c2ccccc2N(c2c(N(c3ccc(-c4ccccc4)cc3)c3ccc(-c4cccc5ccccc45)cc3)ccc3ccccc23)c2ccc(-c3ccccc3)cc21]

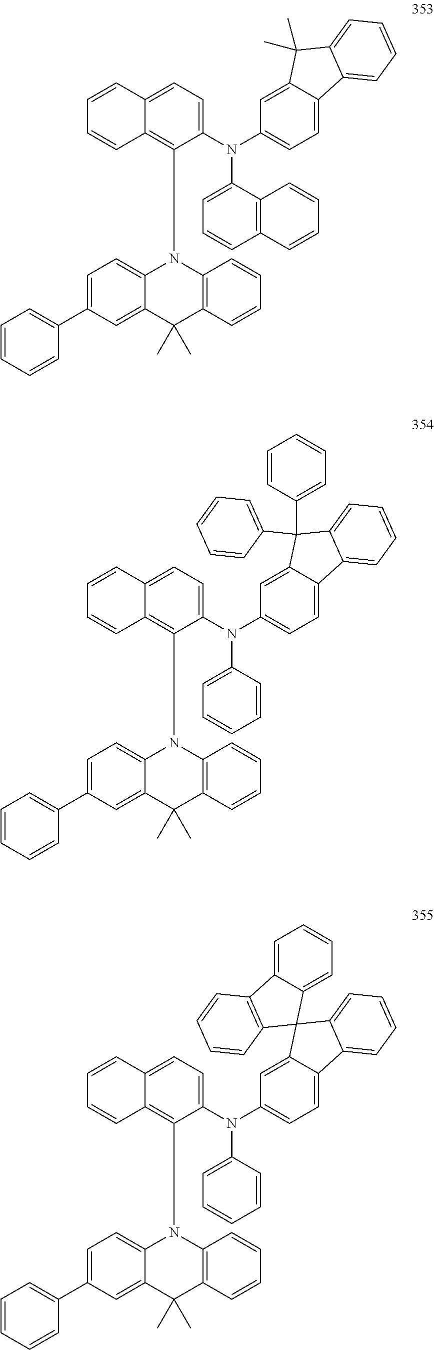 [SMILES: CC1(C)c2ccccc2-c2ccc(N(c3ccc4ccccc4c3N3c4ccccc4C(C)(C)c4cc(-c5ccccc5)ccc43)c3cccc4ccccc34)cc21 ; CC1(C)c2ccccc2N(c2c(N(c3ccccc3)c3ccc4c(c3)C(c3ccccc3)(c3ccccc3)c3ccccc3-4)ccc3ccccc23)c2ccc(-c3ccccc3)cc21 ; CC1(C)c2ccccc2N(c2c(N(c3ccccc3)c3ccc4c(c3)C3(c5ccccc5-c5ccccc53)c3ccccc3-4)ccc3ccccc23)c2ccc(-c3ccccc3)cc21]

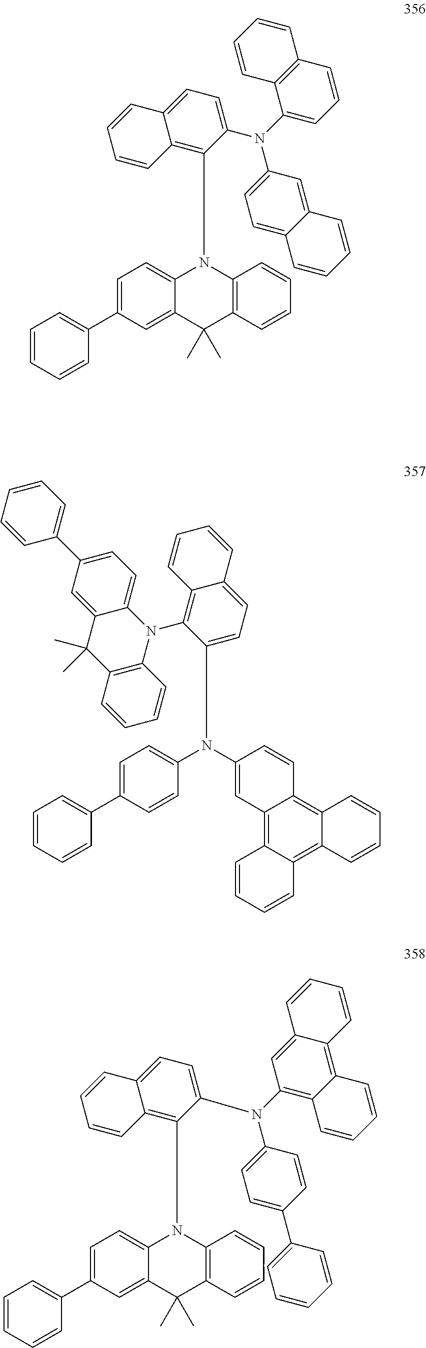 [SMILES: CC1(C)c2ccccc2N(c2c(N(c3ccc4ccccc4c3)c3cccc4ccccc34)ccc3ccccc23)c2ccc(-c3ccccc3)cc21 ; CC1(C)c2ccccc2N(c2c(N(c3ccc(-c4ccccc4)cc3)c3ccc4c5ccccc5c5ccccc5c4c3)ccc3ccccc23)c2ccc(-c3ccccc3)cc21 ; CC1(C)c2ccccc2N(c2c(N(c3ccc(-c4ccccc4)cc3)c3cc4ccccc4c4ccccc34)ccc3ccccc23)c2ccc(-c3ccccc3)cc21]

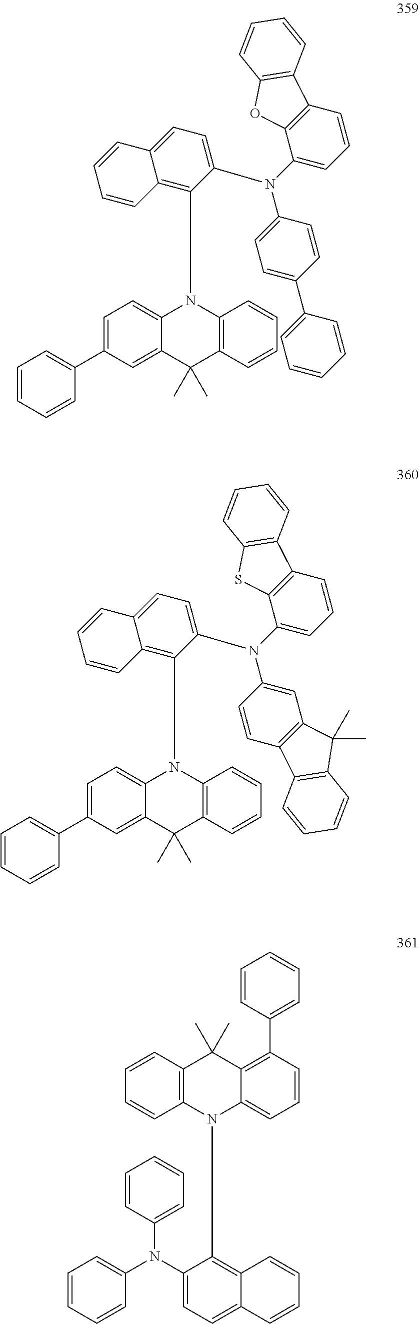 [SMILES: CC1(C)c2ccccc2N(c2c(N(c3ccc(-c4ccccc4)cc3)c3cccc4c3oc3ccccc34)ccc3ccccc23)c2ccc(-c3ccccc3)cc21 ; CC1(C)c2ccccc2-c2ccc(N(c3ccc4ccccc4c3N3c4ccccc4C(C)(C)c4cc(-c5ccccc5)ccc43)c3cccc4c3sc3ccccc34)cc21 ; CC1(C)c2ccccc2N(c2c(N(c3ccccc3)c3ccccc3)ccc3ccccc23)c2cccc(-c3ccccc3)c21]

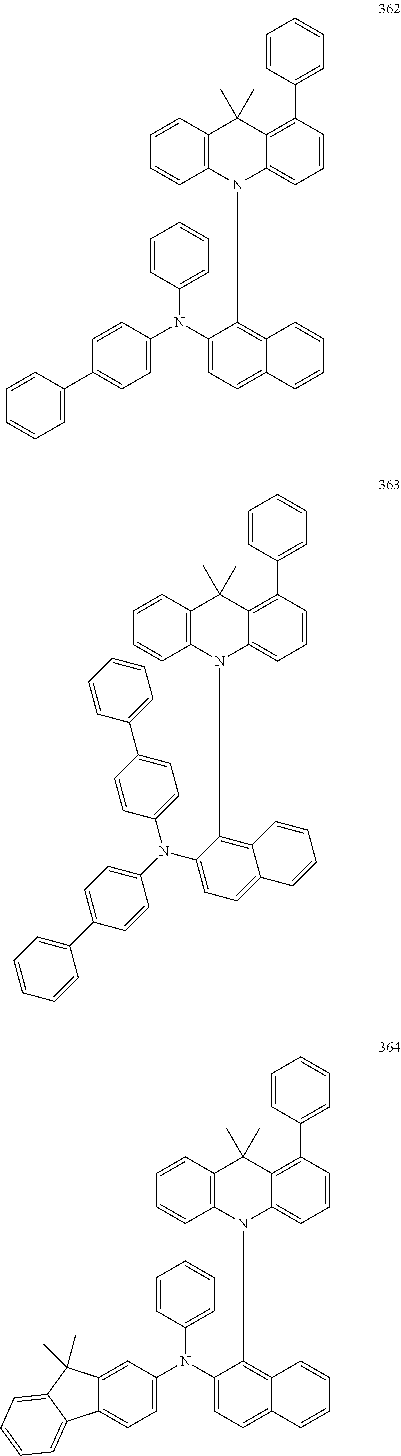 [SMILES: CC1(C)c2ccccc2N(c2c(N(c3ccccc3)c3ccc(-c4ccccc4)cc3)ccc3ccccc23)c2cccc(-c3ccccc3)c21 ; CC1(C)c2ccccc2N(c2c(N(c3ccc(-c4ccccc4)cc3)c3ccc(-c4ccccc4)cc3)ccc3ccccc23)c2cccc(-c3ccccc3)c21 ; CC1(C)c2ccccc2-c2ccc(N(c3ccccc3)c3ccc4ccccc4c3N3c4ccccc4C(C)(C)c4c(-c5ccccc5)cccc43)cc21]

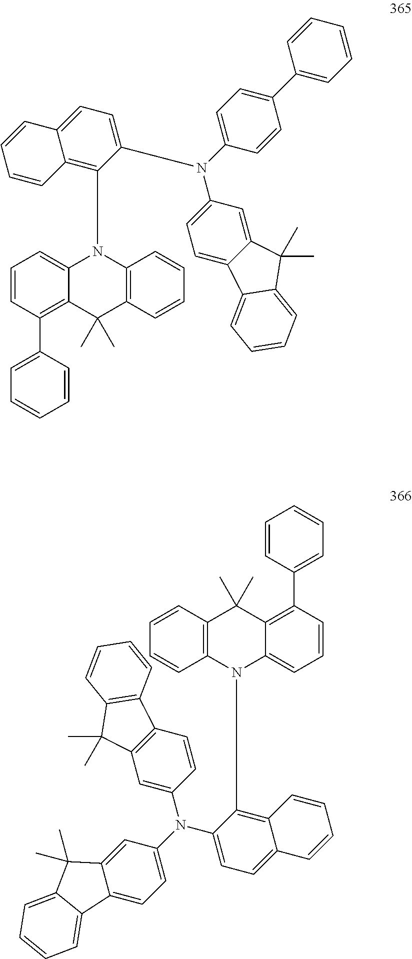 [SMILES: CC1(C)c2ccccc2-c2ccc(N(c3ccc(-c4ccccc4)cc3)c3ccc4ccccc4c3N3c4ccccc4C(C)(C)c4c(-c5ccccc5)cccc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4c(-c5ccccc5)cccc43)cc21]

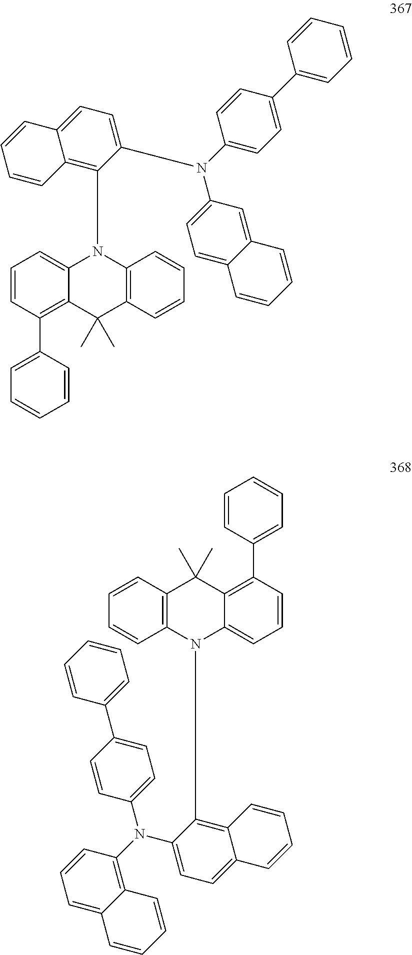 [SMILES: CC1(C)c2ccccc2N(c2c(N(c3ccc(-c4ccccc4)cc3)c3ccc4ccccc4c3)ccc3ccccc23)c2cccc(-c3ccccc3)c21 ; CC1(C)c2ccccc2N(c2c(N(c3ccc(-c4ccccc4)cc3)c3cccc4ccccc34)ccc3ccccc23)c2cccc(-c3ccccc3)c21]

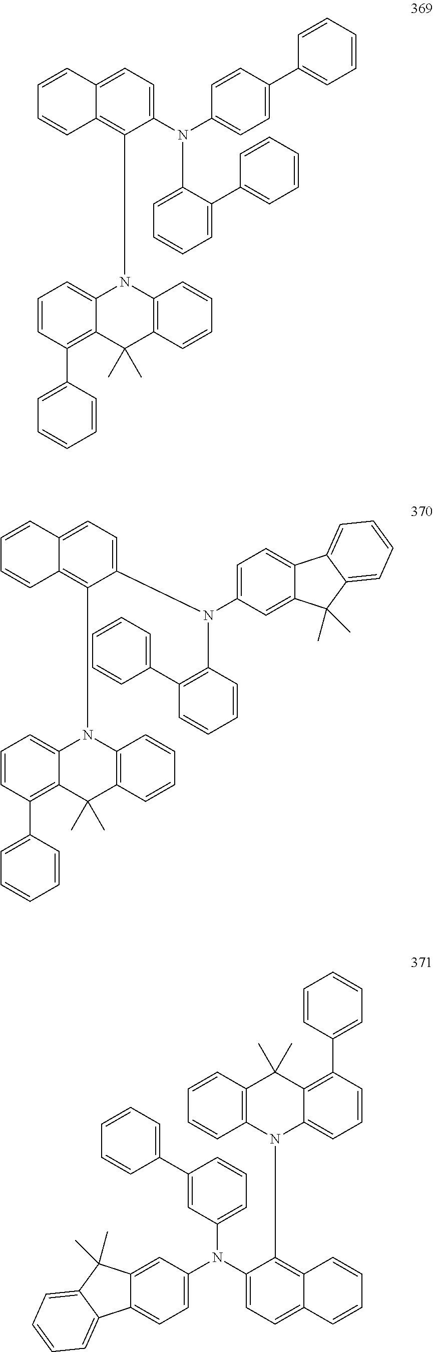 [SMILES: CC1(C)c2ccccc2N(c2c(N(c3ccc(-c4ccccc4)cc3)c3ccccc3-c3ccccc3)ccc3ccccc23)c2cccc(-c3ccccc3)c21 ; CC1(C)c2ccccc2-c2ccc(N(c3ccccc3-c3ccccc3)c3ccc4ccccc4c3N3c4ccccc4C(C)(C)c4c(-c5ccccc5)cccc43)cc21 ; CC1(C)c2ccccc2-c2ccc(N(c3cccc(-c4ccccc4)c3)c3ccc4ccccc4c3N3c4ccccc4C(C)(C)c4c(-c5ccccc5)cccc43)cc21]

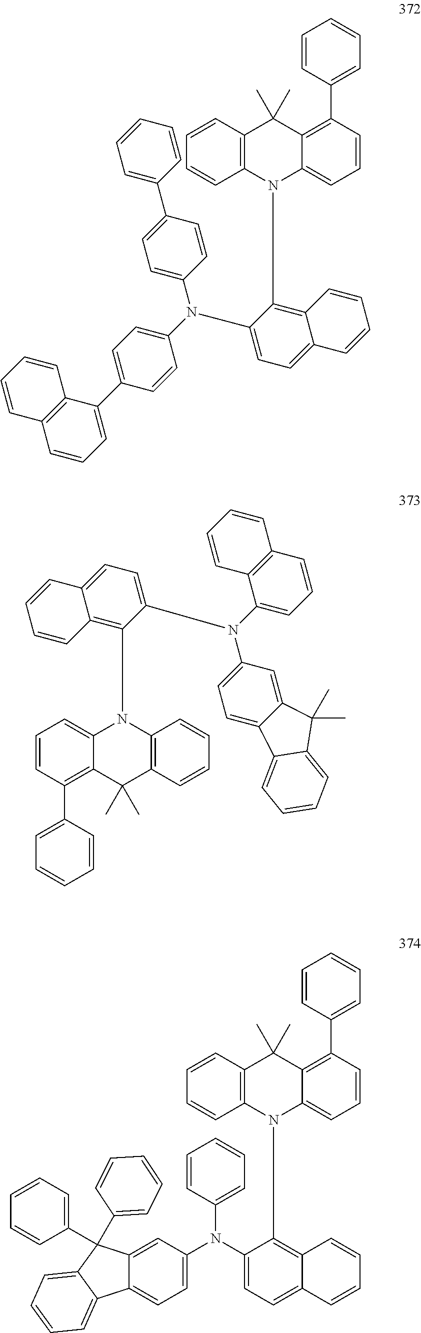 [SMILES: CC1(C)c2ccccc2N(c2c(N(c3ccc(-c4ccccc4)cc3)c3ccc(-c4cccc5ccccc45)cc3)ccc3ccccc23)c2cccc(-c3ccccc3)c21 ; CC1(C)c2ccccc2-c2ccc(N(c3ccc4ccccc4c3N3c4ccccc4C(C)(C)c4c(-c5ccccc5)cccc43)c3cccc4ccccc34)cc21 ; CC1(C)c2ccccc2N(c2c(N(c3ccccc3)c3ccc4c(c3)C(c3ccccc3)(c3ccccc3)c3ccccc3-4)ccc3ccccc23)c2cccc(-c3ccccc3)c21]

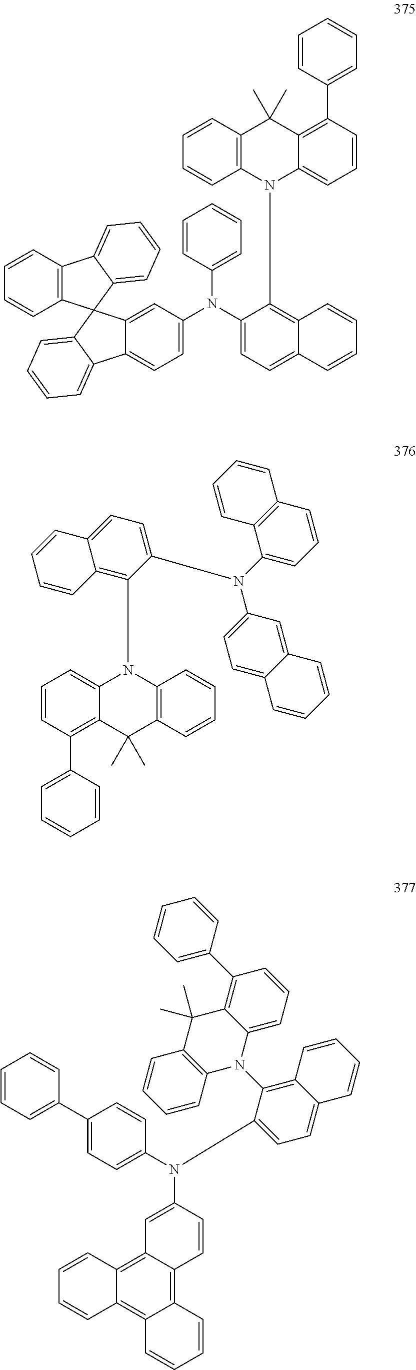 [SMILES: CC1(C)c2ccccc2N(c2c(N(c3ccccc3)c3ccc4c(c3)C3(c5ccccc5-c5ccccc53)c3ccccc3-4)ccc3ccccc23)c2cccc(-c3ccccc3)c21 ; CC1(C)c2ccccc2N(c2c(N(c3ccc4ccccc4c3)c3cccc4ccccc34)ccc3ccccc23)c2cccc(-c3ccccc3)c21 ; CC1(C)c2ccccc2N(c2c(N(c3ccc(-c4ccccc4)cc3)c3ccc4c5ccccc5c5ccccc5c4c3)ccc3ccccc23)c2cccc(-c3ccccc3)c21]

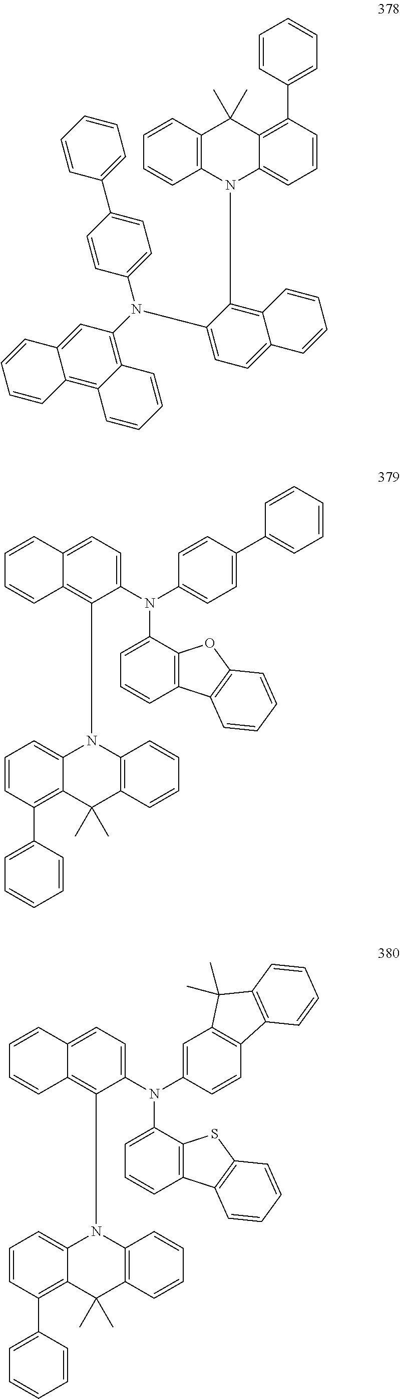 [SMILES: CC1(C)c2ccccc2N(c2c(N(c3ccc(-c4ccccc4)cc3)c3cc4ccccc4c4ccccc34)ccc3ccccc23)c2cccc(-c3ccccc3)c21 ; CC1(C)c2ccccc2N(c2c(N(c3ccc(-c4ccccc4)cc3)c3cccc4c3oc3ccccc34)ccc3ccccc23)c2cccc(-c3ccccc3)c21 ; CC1(C)c2ccccc2-c2ccc(N(c3ccc4ccccc4c3N3c4ccccc4C(C)(C)c4c(-c5ccccc5)cccc43)c3cccc4c3sc3ccccc34)cc21]

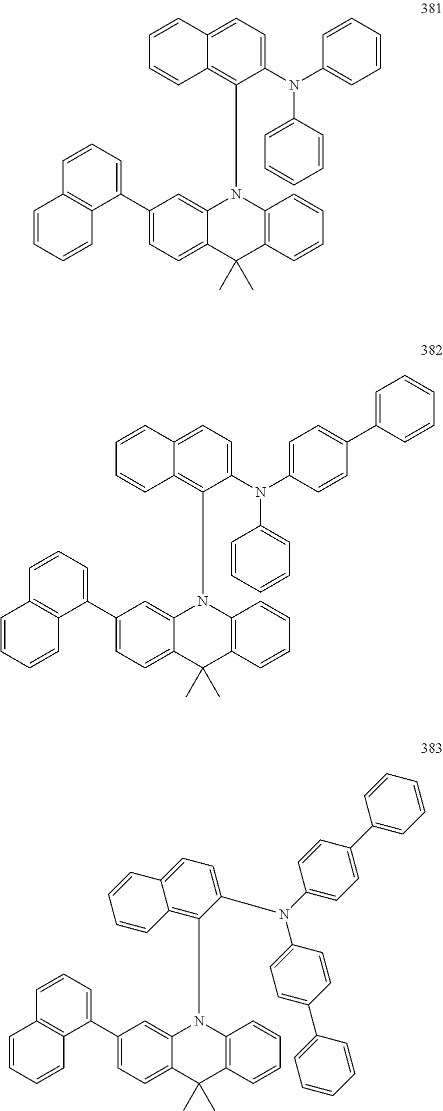 [SMILES: CC1(C)c2ccccc2N(c2c(N(c3ccccc3)c3ccccc3)ccc3ccccc23)c2cc(-c3cccc4ccccc34)ccc21 ; CC1(C)c2ccccc2N(c2c(N(c3ccccc3)c3ccc(-c4ccccc4)cc3)ccc3ccccc23)c2cc(-c3cccc4ccccc34)ccc21 ; CC1(C)c2ccccc2N(c2c(N(c3ccc(-c4ccccc4)cc3)c3ccc(-c4ccccc4)cc3)ccc3ccccc23)c2cc(-c3cccc4ccccc34)ccc21]

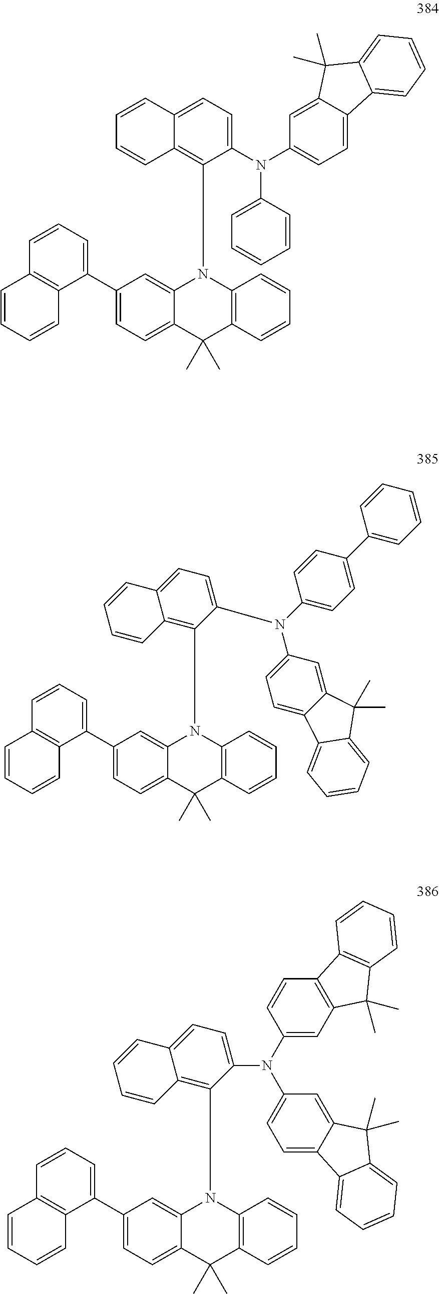 [SMILES: CC1(C)c2ccccc2-c2ccc(N(c3ccccc3)c3ccc4ccccc4c3N3c4ccccc4C(C)(C)c4ccc(-c5cccc6ccccc56)cc43)cc21 ; CC1(C)c2ccccc2-c2ccc(N(c3ccc(-c4ccccc4)cc3)c3ccc4ccccc4c3N3c4ccccc4C(C)(C)c4ccc(-c5cccc6ccccc56)cc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4ccc(-c5cccc6ccccc56)cc43)cc21]

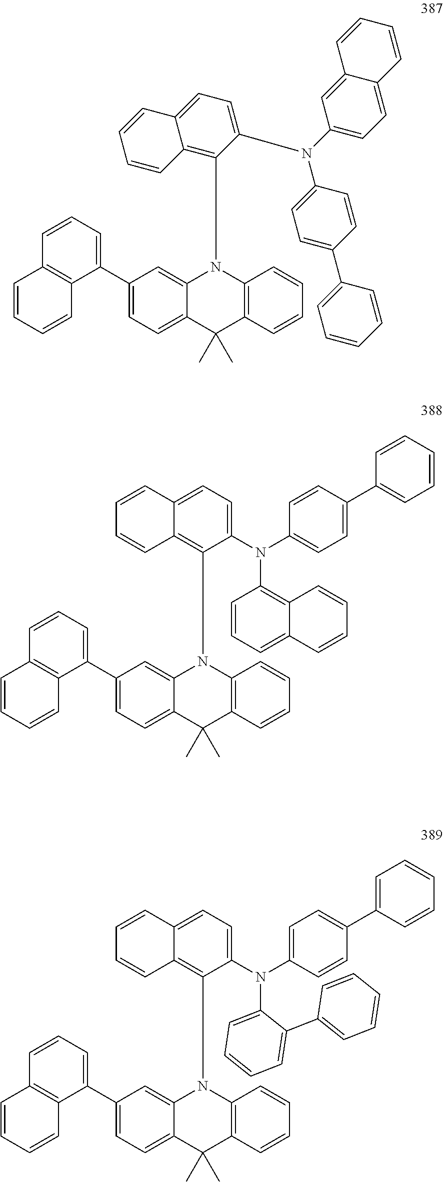 [SMILES: CC1(C)c2ccccc2N(c2c(N(c3ccc(-c4ccccc4)cc3)c3ccc4ccccc4c3)ccc3ccccc23)c2cc(-c3cccc4ccccc34)ccc21 ; CC1(C)c2ccccc2N(c2c(N(c3ccc(-c4ccccc4)cc3)c3cccc4ccccc34)ccc3ccccc23)c2cc(-c3cccc4ccccc34)ccc21 ; CC1(C)c2ccccc2N(c2c(N(c3ccc(-c4ccccc4)cc3)c3ccccc3-c3ccccc3)ccc3ccccc23)c2cc(-c3cccc4ccccc34)ccc21]

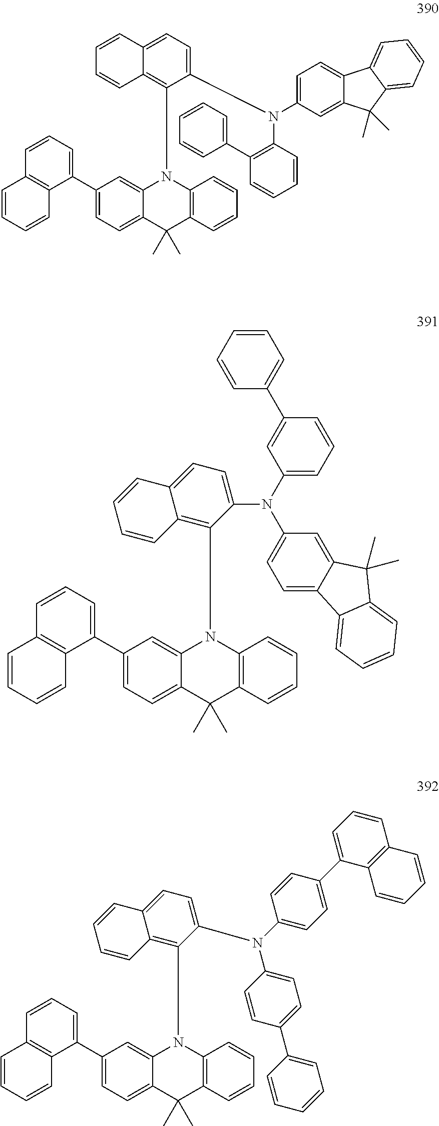 [SMILES: CC1(C)c2ccccc2-c2ccc(N(c3ccccc3-c3ccccc3)c3ccc4ccccc4c3N3c4ccccc4C(C)(C)c4ccc(-c5cccc6ccccc56)cc43)cc21 ; CC1(C)c2ccccc2-c2ccc(N(c3cccc(-c4ccccc4)c3)c3ccc4ccccc4c3N3c4ccccc4C(C)(C)c4ccc(-c5cccc6ccccc56)cc43)cc21 ; CC1(C)c2ccccc2N(c2c(N(c3ccc(-c4ccccc4)cc3)c3ccc(-c4cccc5ccccc45)cc3)ccc3ccccc23)c2cc(-c3cccc4ccccc34)ccc21]

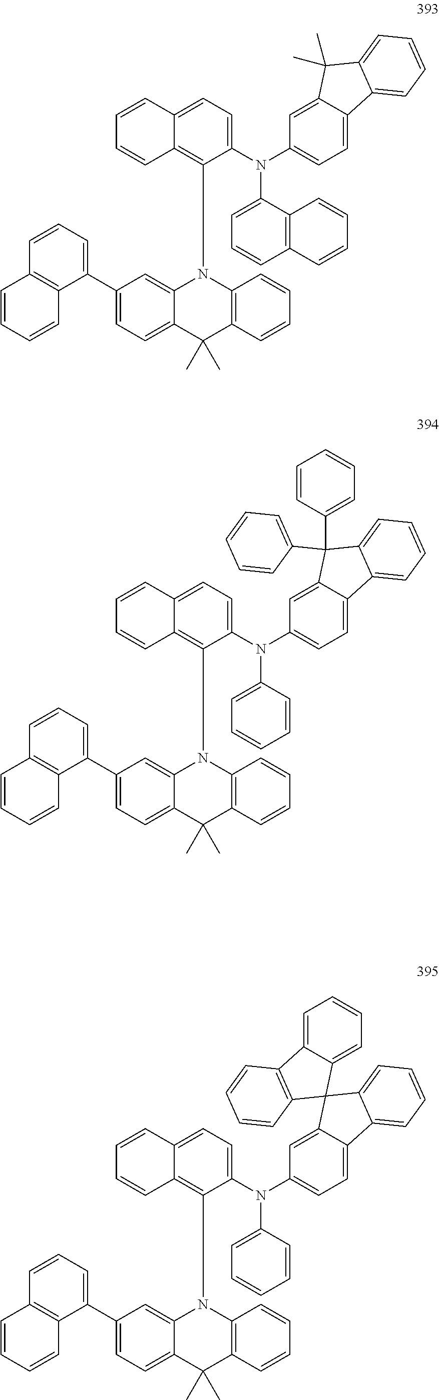 [SMILES: CC1(C)c2ccccc2-c2ccc(N(c3ccc4ccccc4c3N3c4ccccc4C(C)(C)c4ccc(-c5cccc6ccccc56)cc43)c3cccc4ccccc34)cc21 ; CC1(C)c2ccccc2N(c2c(N(c3ccccc3)c3ccc4c(c3)C(c3ccccc3)(c3ccccc3)c3ccccc3-4)ccc3ccccc23)c2cc(-c3cccc4ccccc34)ccc21 ; CC1(C)c2ccccc2N(c2c(N(c3ccccc3)c3ccc4c(c3)C3(c5ccccc5-c5ccccc53)c3ccccc3-4)ccc3ccccc23)c2cc(-c3cccc4ccccc34)ccc21]

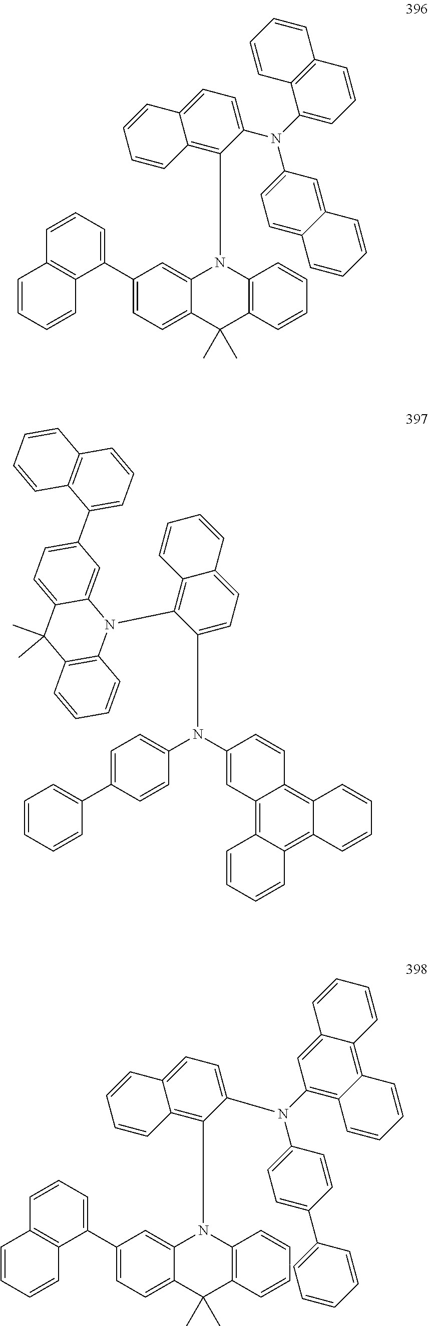 [SMILES: CC1(C)c2ccccc2N(c2c(N(c3ccc4ccccc4c3)c3cccc4ccccc34)ccc3ccccc23)c2cc(-c3cccc4ccccc34)ccc21 ; CC1(C)c2ccccc2N(c2c(N(c3ccc(-c4ccccc4)cc3)c3ccc4c5ccccc5c5ccccc5c4c3)ccc3ccccc23)c2cc(-c3cccc4ccccc34)ccc21 ; CC1(C)c2ccccc2N(c2c(N(c3ccc(-c4ccccc4)cc3)c3cc4ccccc4c4ccccc34)ccc3ccccc23)c2cc(-c3cccc4ccccc34)ccc21]

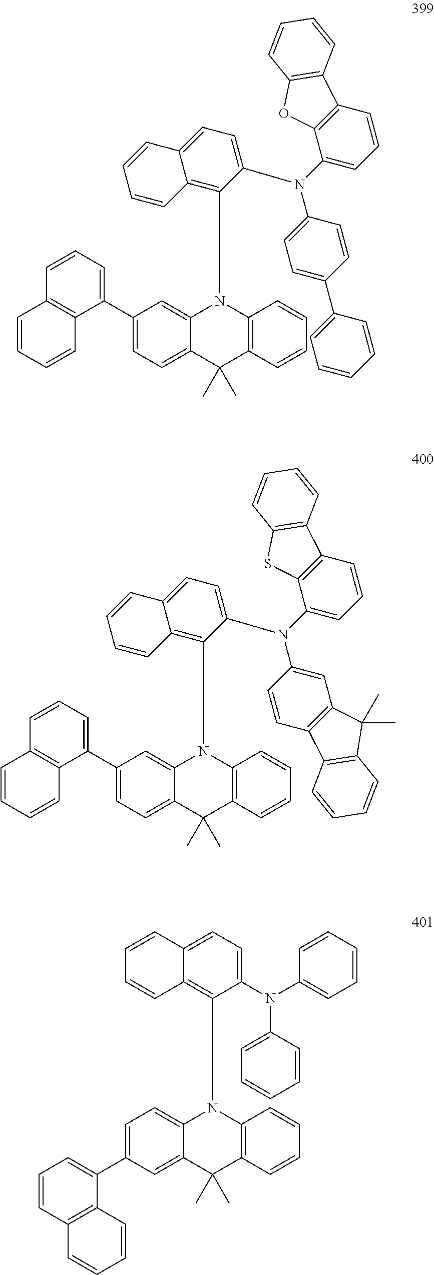 [SMILES: CC1(C)c2ccccc2N(c2c(N(c3ccc(-c4ccccc4)cc3)c3cccc4c3oc3ccccc34)ccc3ccccc23)c2cc(-c3cccc4ccccc34)ccc21 ; CC1(C)c2ccccc2-c2ccc(N(c3ccc4ccccc4c3N3c4ccccc4C(C)(C)c4ccc(-c5cccc6ccccc56)cc43)c3cccc4c3sc3ccccc34)cc21 ; CC1(C)c2ccccc2N(c2c(N(c3ccccc3)c3ccccc3)ccc3ccccc23)c2ccc(-c3cccc4ccccc34)cc21]

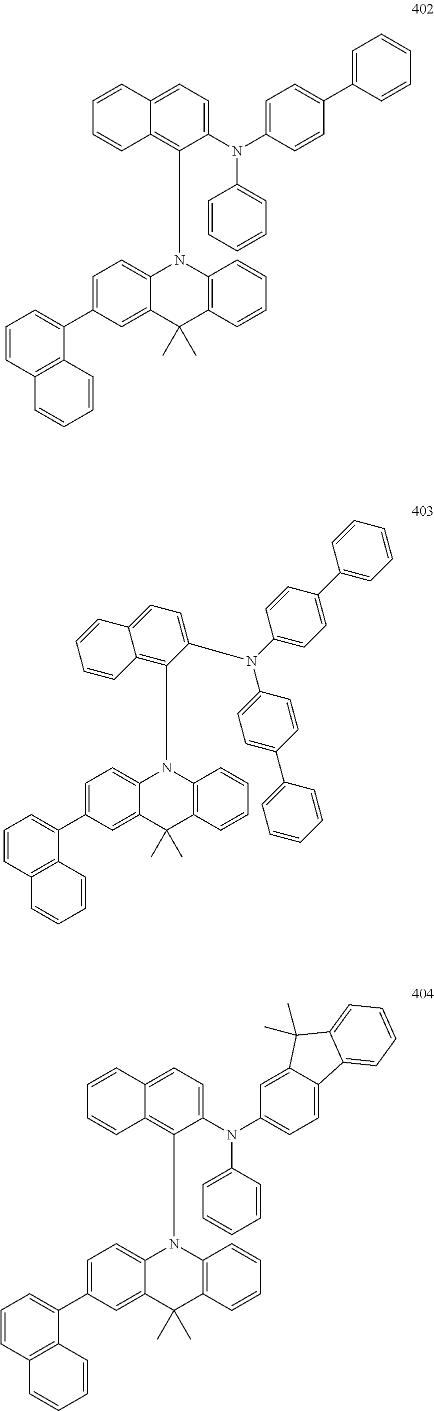 [SMILES: CC1(C)c2ccccc2N(c2c(N(c3ccccc3)c3ccc(-c4ccccc4)cc3)ccc3ccccc23)c2ccc(-c3cccc4ccccc34)cc21 ; CC1(C)c2ccccc2N(c2c(N(c3ccc(-c4ccccc4)cc3)c3ccc(-c4ccccc4)cc3)ccc3ccccc23)c2ccc(-c3cccc4ccccc34)cc21 ; CC1(C)c2ccccc2-c2ccc(N(c3ccccc3)c3ccc4ccccc4c3N3c4ccccc4C(C)(C)c4cc(-c5cccc6ccccc56)ccc43)cc21]

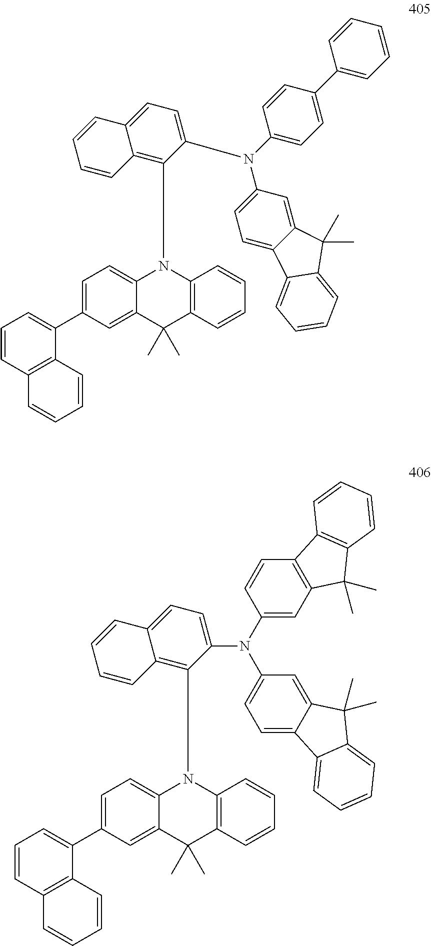 [SMILES: CC1(C)c2ccccc2-c2ccc(N(c3ccc(-c4ccccc4)cc3)c3ccc4ccccc4c3N3c4ccccc4C(C)(C)c4cc(-c5cccc6ccccc56)ccc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4cc(-c5cccc6ccccc56)ccc43)cc21]

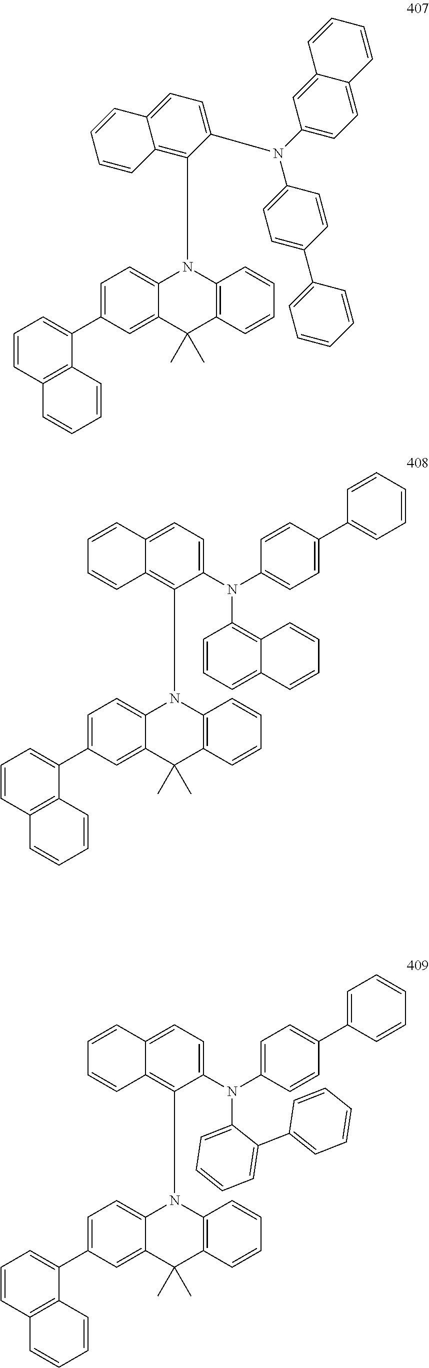 [SMILES: CC1(C)c2ccccc2N(c2c(N(c3ccc(-c4ccccc4)cc3)c3ccc4ccccc4c3)ccc3ccccc23)c2ccc(-c3cccc4ccccc34)cc21 ; CC1(C)c2ccccc2N(c2c(N(c3ccc(-c4ccccc4)cc3)c3cccc4ccccc34)ccc3ccccc23)c2ccc(-c3cccc4ccccc34)cc21 ; CC1(C)c2ccccc2N(c2c(N(c3ccc(-c4ccccc4)cc3)c3ccccc3-c3ccccc3)ccc3ccccc23)c2ccc(-c3cccc4ccccc34)cc21]

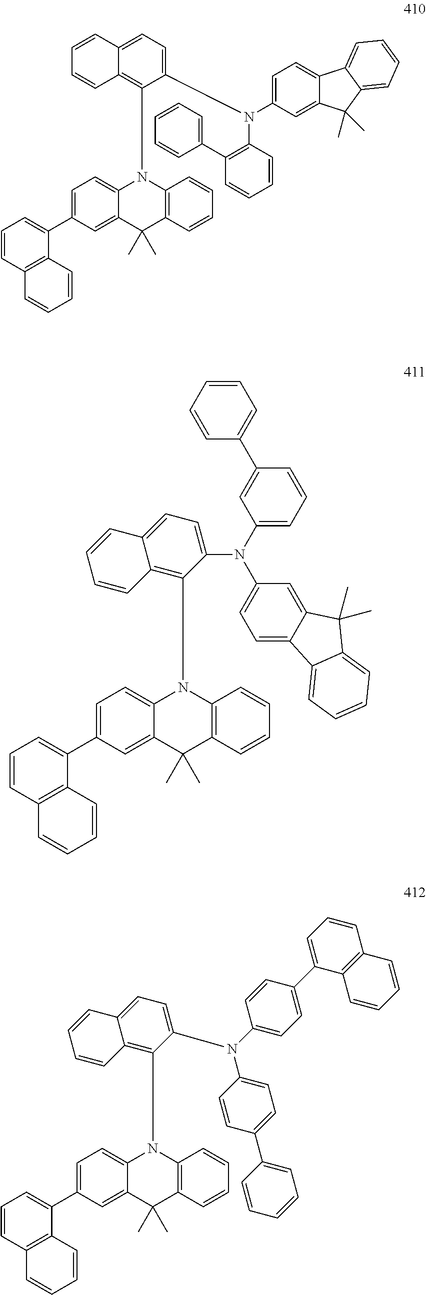 [SMILES: CC1(C)c2ccccc2-c2ccc(N(c3ccccc3-c3ccccc3)c3ccc4ccccc4c3N3c4ccccc4C(C)(C)c4cc(-c5cccc6ccccc56)ccc43)cc21 ; CC1(C)c2ccccc2-c2ccc(N(c3cccc(-c4ccccc4)c3)c3ccc4ccccc4c3N3c4ccccc4C(C)(C)c4cc(-c5cccc6ccccc56)ccc43)cc21 ; CC1(C)c2ccccc2N(c2c(N(c3ccc(-c4ccccc4)cc3)c3ccc(-c4cccc5ccccc45)cc3)ccc3ccccc23)c2ccc(-c3cccc4ccccc34)cc21]

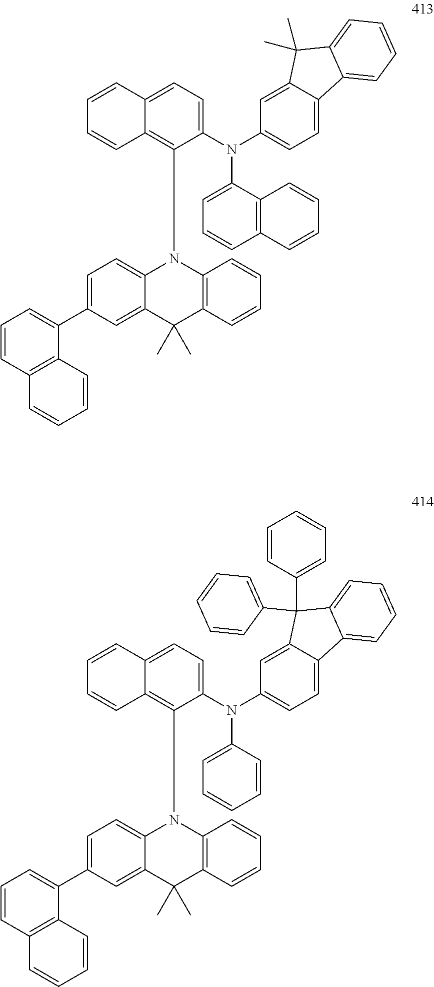 [SMILES: CC1(C)c2ccccc2-c2ccc(N(c3ccc4ccccc4c3N3c4ccccc4C(C)(C)c4cc(-c5cccc6ccccc56)ccc43)c3cccc4ccccc34)cc21 ; CC1(C)c2ccccc2N(c2c(N(c3ccccc3)c3ccc4c(c3)C(c3ccccc3)(c3ccccc3)c3ccccc3-4)ccc3ccccc23)c2ccc(-c3cccc4ccccc34)cc21]

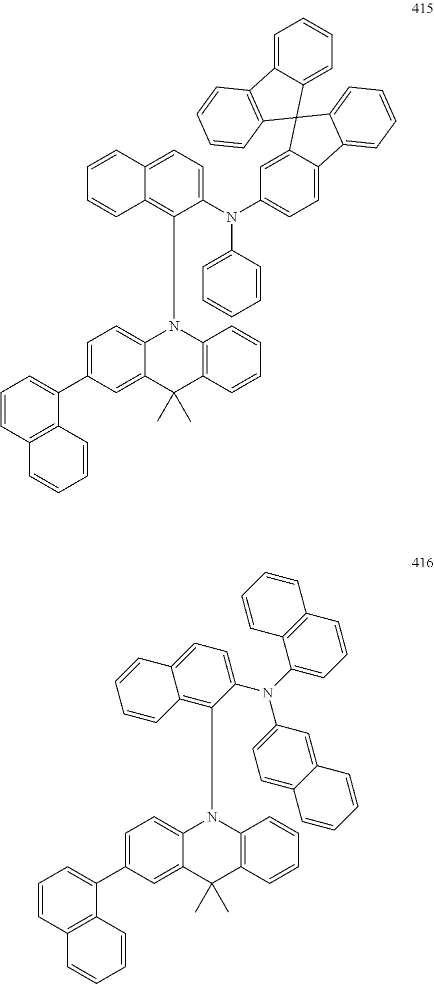 [SMILES: CC1(C)c2ccccc2N(c2c(N(c3ccccc3)c3ccc4c(c3)C3(c5ccccc5-c5ccccc53)c3ccccc3-4)ccc3ccccc23)c2ccc(-c3cccc4ccccc34)cc21 ; CC1(C)c2ccccc2N(c2c(N(c3ccc4ccccc4c3)c3cccc4ccccc34)ccc3ccccc23)c2ccc(-c3cccc4ccccc34)cc21]

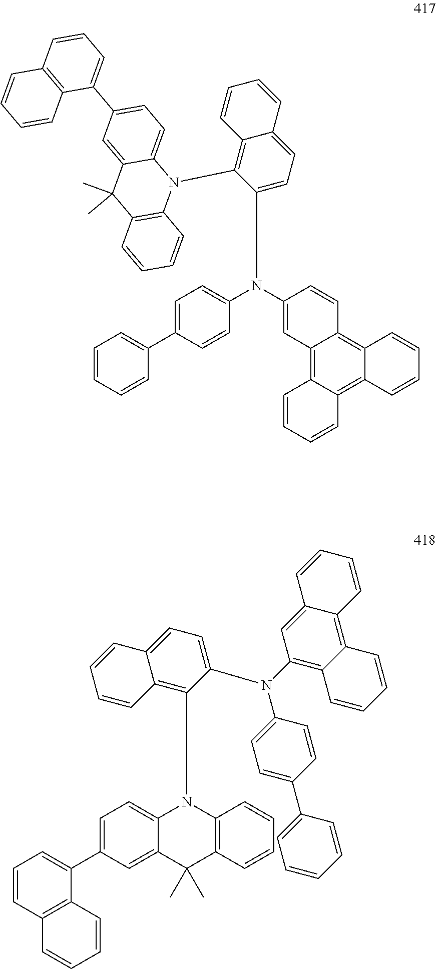 [SMILES: CC1(C)c2ccccc2N(c2c(N(c3ccc(-c4ccccc4)cc3)c3ccc4c5ccccc5c5ccccc5c4c3)ccc3ccccc23)c2ccc(-c3cccc4ccccc34)cc21 ; CC1(C)c2ccccc2N(c2c(N(c3ccc(-c4ccccc4)cc3)c3cc4ccccc4c4ccccc34)ccc3ccccc23)c2ccc(-c3cccc4ccccc34)cc21]

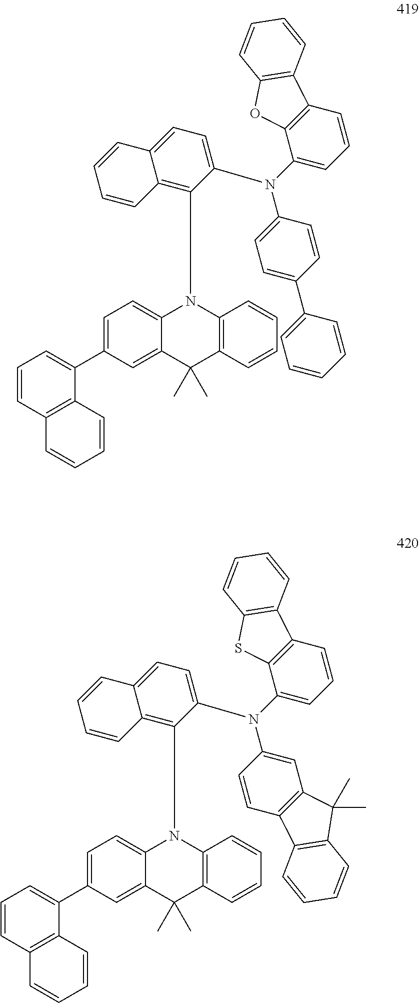 [SMILES: CC1(C)c2ccccc2N(c2c(N(c3ccc(-c4ccccc4)cc3)c3cccc4c3oc3ccccc34)ccc3ccccc23)c2ccc(-c3cccc4ccccc34)cc21 ; CC1(C)c2ccccc2-c2ccc(N(c3ccc4ccccc4c3N3c4ccccc4C(C)(C)c4cc(-c5cccc6ccccc56)ccc43)c3cccc4c3sc3ccccc34)cc21]

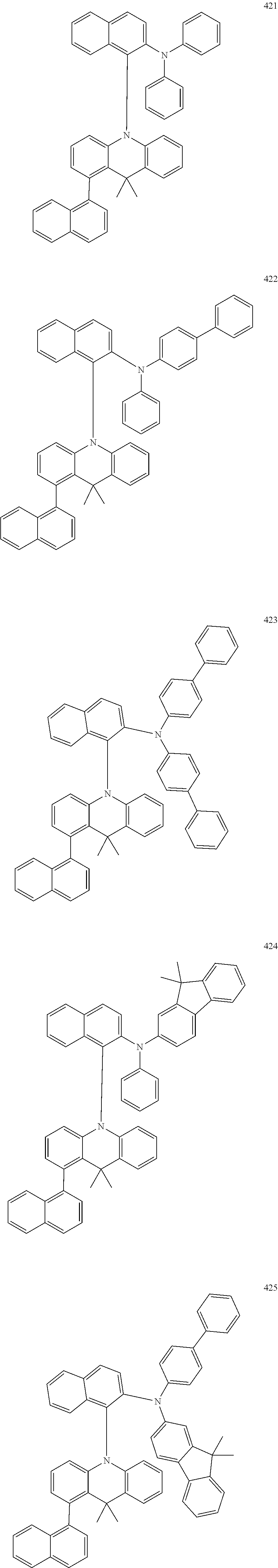 [SMILES: CC1(C)c2ccccc2N(c2c(N(c3ccccc3)c3ccccc3)ccc3ccccc23)c2cccc(-c3cccc4ccccc34)c21 ; CC1(C)c2ccccc2N(c2c(N(c3ccccc3)c3ccc(-c4ccccc4)cc3)ccc3ccccc23)c2cccc(-c3cccc4ccccc34)c21 ; CC1(C)c2ccccc2N(c2c(N(c3ccc(-c4ccccc4)cc3)c3ccc(-c4ccccc4)cc3)ccc3ccccc23)c2cccc(-c3cccc4ccccc34)c21 ; CC1(C)c2ccccc2-c2ccc(N(c3ccccc3)c3ccc4ccccc4c3N3c4ccccc4C(C)(C)c4c(-c5cccc6ccccc56)cccc43)cc21 ; CC1(C)c2ccccc2-c2ccc(N(c3ccc(-c4ccccc4)cc3)c3ccc4ccccc4c3N3c4ccccc4C(C)(C)c4c(-c5cccc6ccccc56)cccc43)cc21]

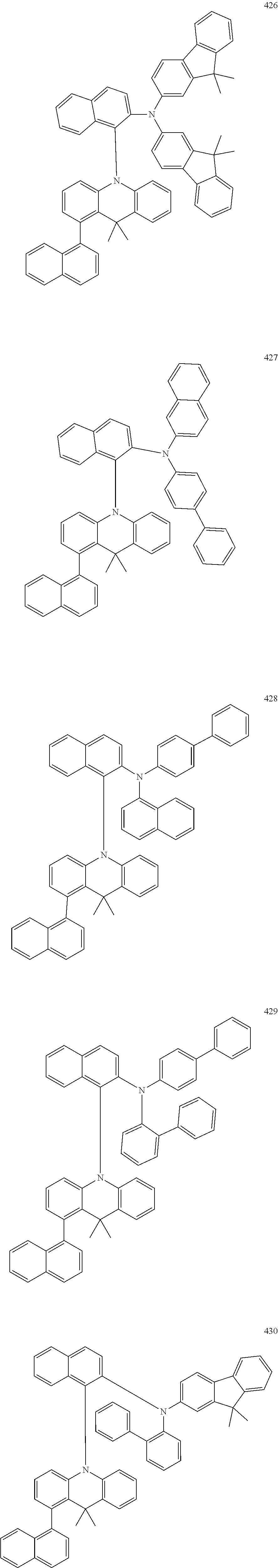

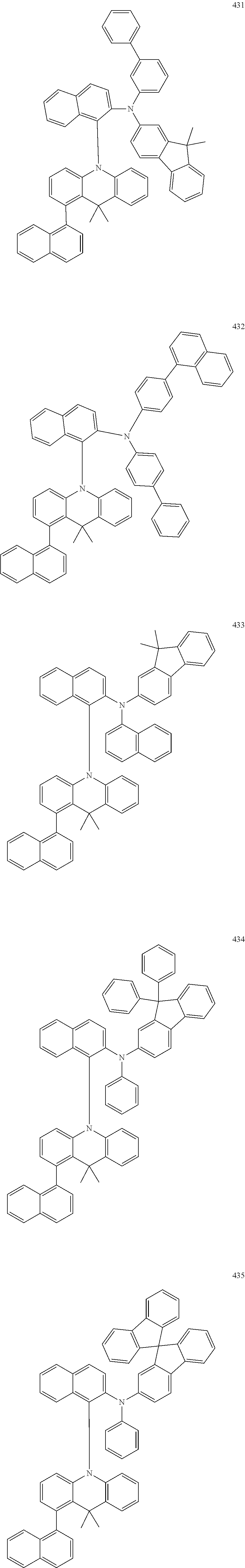

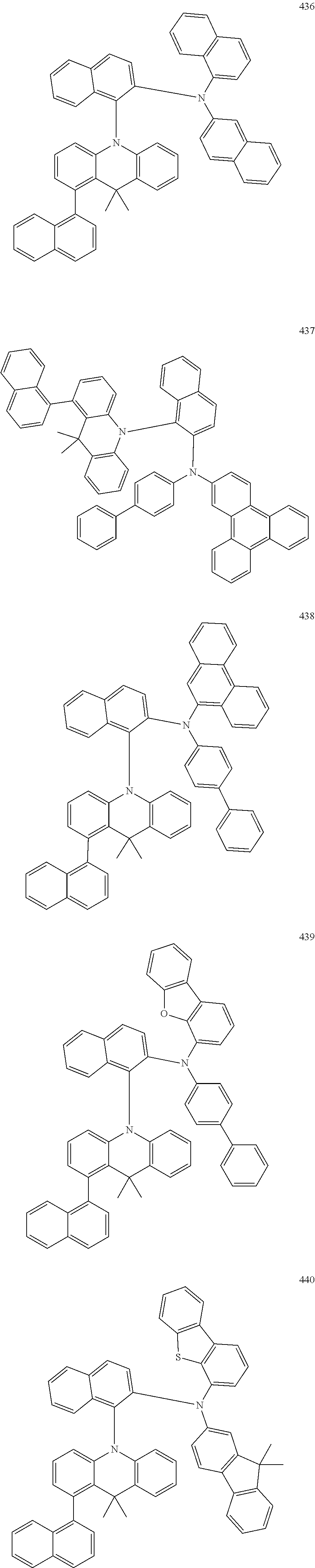

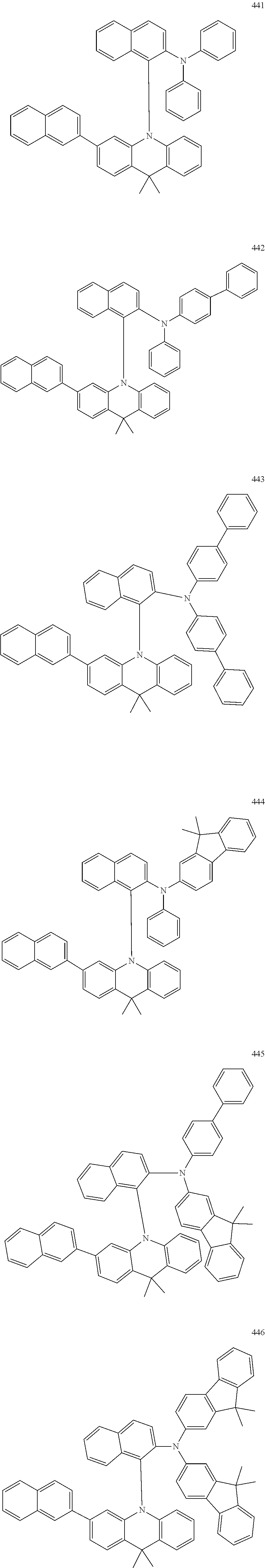

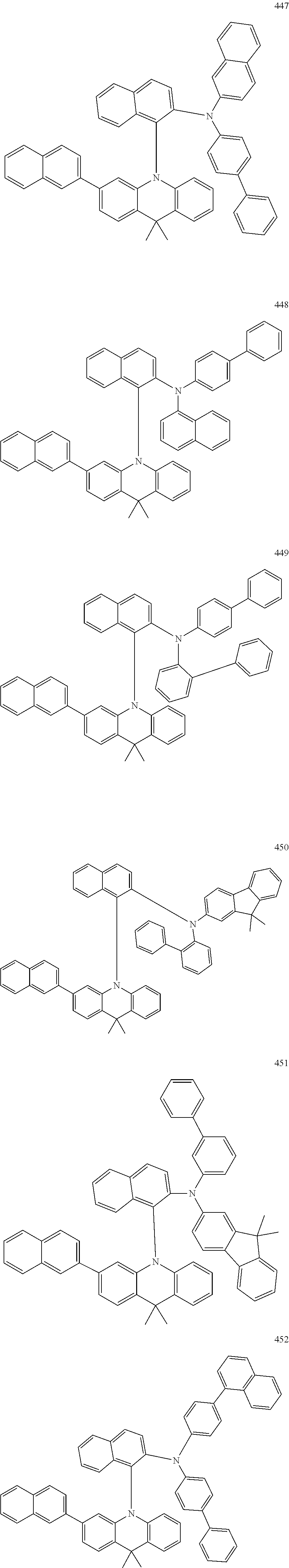

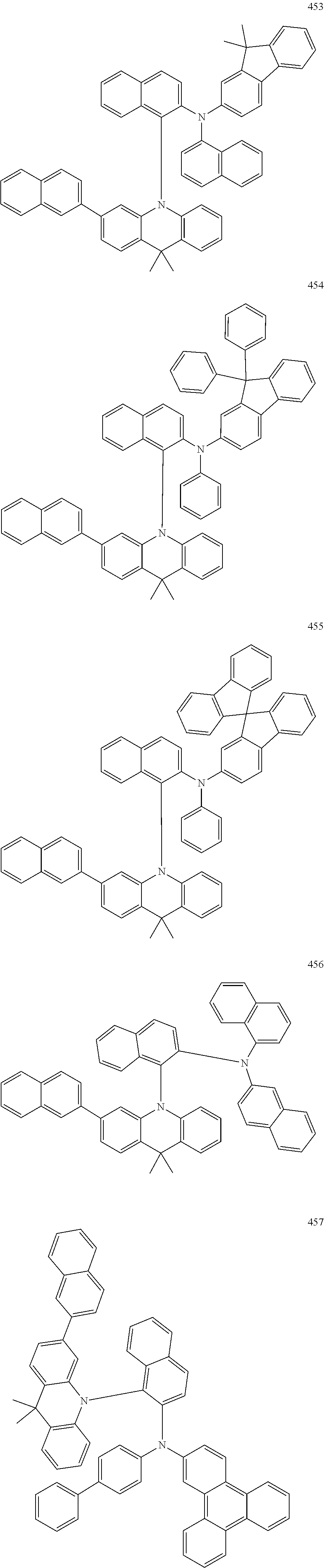

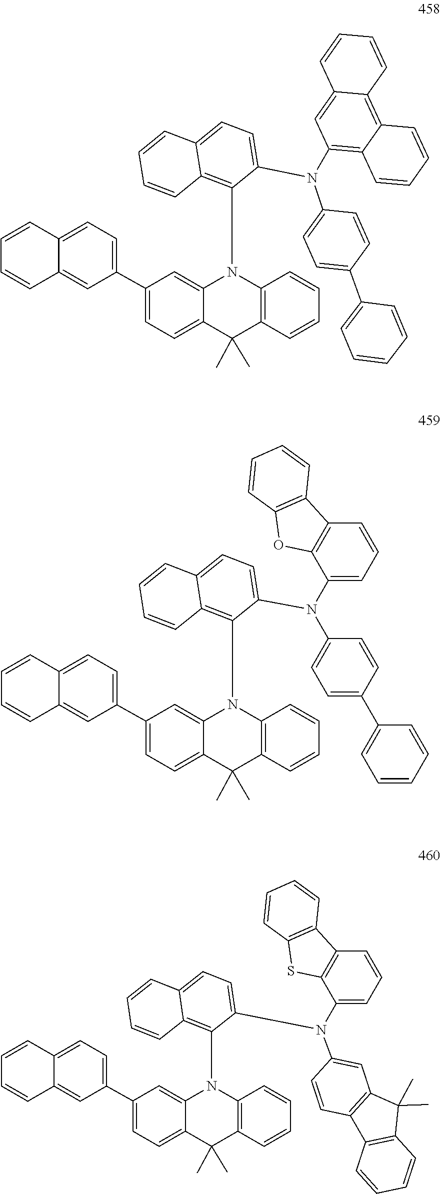 [SMILES: CC1(C)c2ccccc2N(c2c(N(c3ccc(-c4ccccc4)cc3)c3cc4ccccc4c4ccccc34)ccc3ccccc23)c2cc(-c3ccc4ccccc4c3)ccc21 ; CC1(C)c2ccccc2N(c2c(N(c3ccc(-c4ccccc4)cc3)c3cccc4c3oc3ccccc34)ccc3ccccc23)c2cc(-c3ccc4ccccc4c3)ccc21 ; CC1(C)c2ccccc2-c2ccc(N(c3ccc4ccccc4c3N3c4ccccc4C(C)(C)c4ccc(-c5ccc6ccccc6c5)cc43)c3cccc4c3sc3ccccc34)cc21]

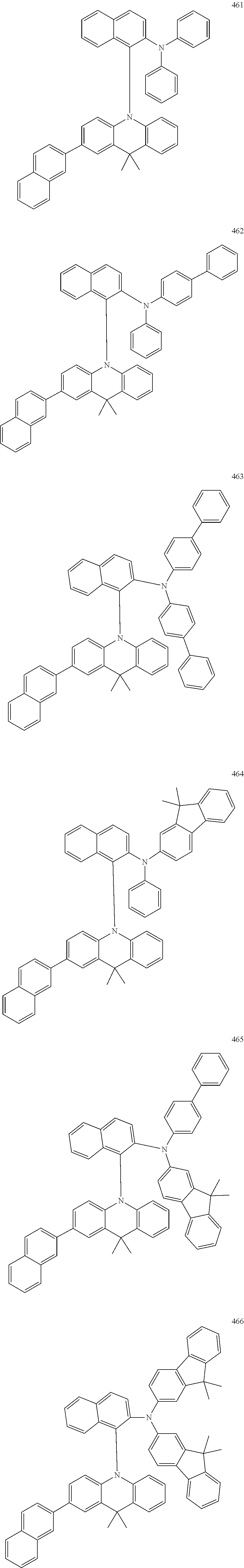

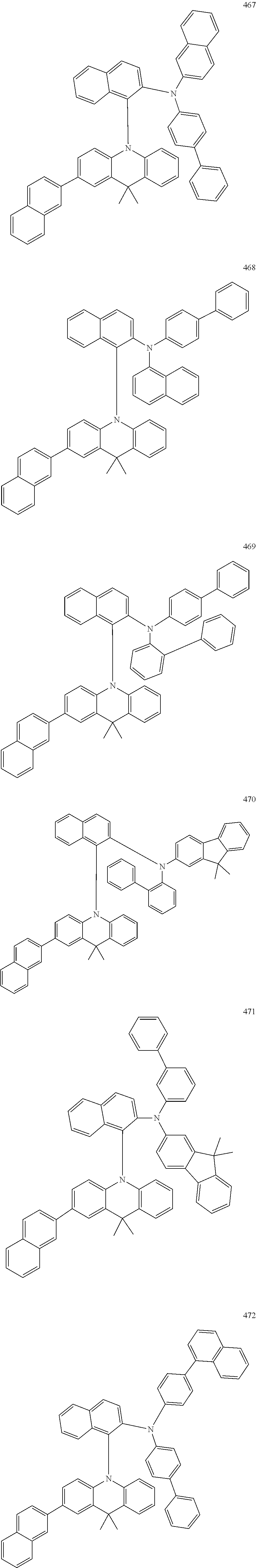

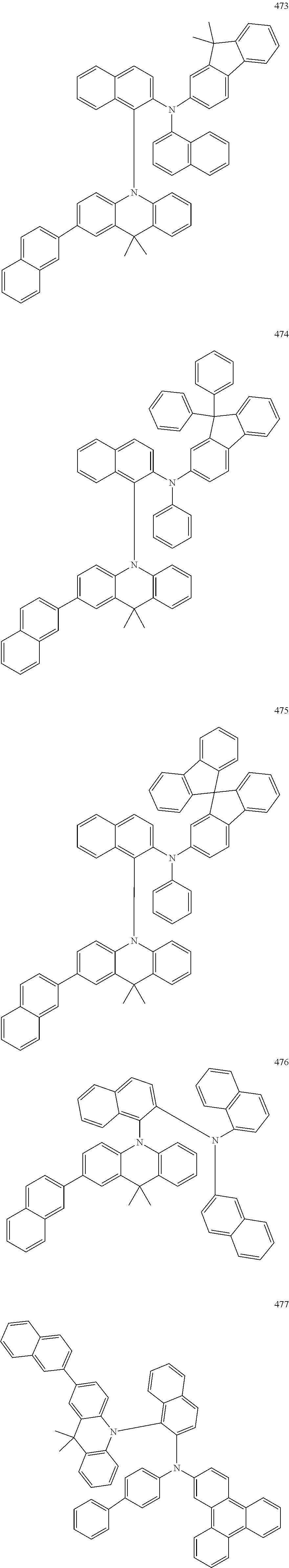

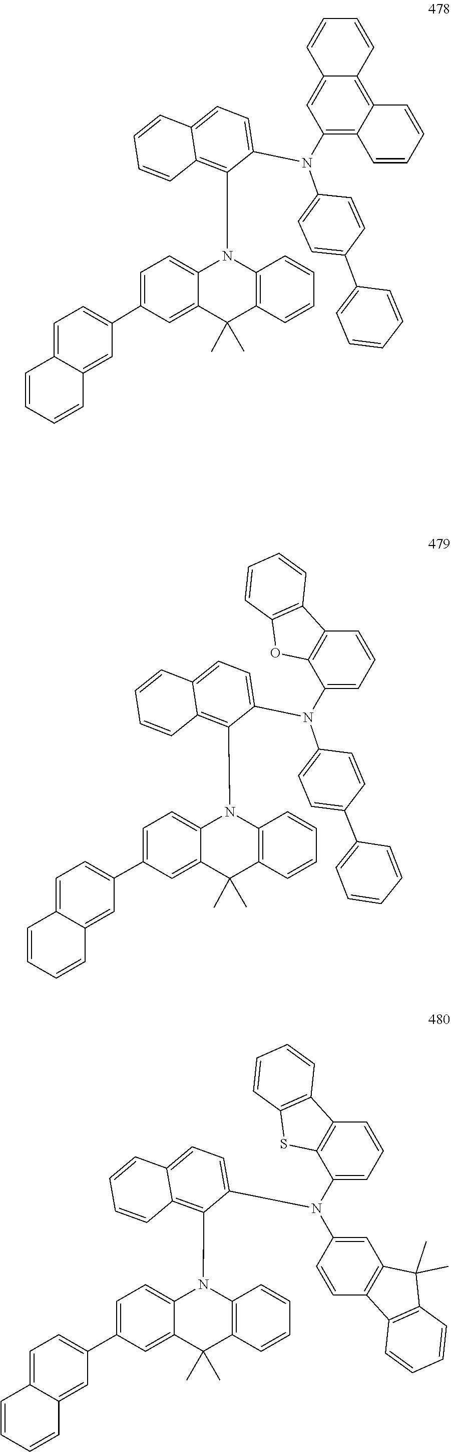 [SMILES: CC1(C)c2ccccc2N(c2c(N(c3ccc(-c4ccccc4)cc3)c3cc4ccccc4c4ccccc34)ccc3ccccc23)c2ccc(-c3ccc4ccccc4c3)cc21 ; CC1(C)c2ccccc2N(c2c(N(c3ccc(-c4ccccc4)cc3)c3cccc4c3oc3ccccc34)ccc3ccccc23)c2ccc(-c3ccc4ccccc4c3)cc21 ; CC1(C)c2ccccc2-c2ccc(N(c3ccc4ccccc4c3N3c4ccccc4C(C)(C)c4cc(-c5ccc6ccccc6c5)ccc43)c3cccc4c3sc3ccccc34)cc21]

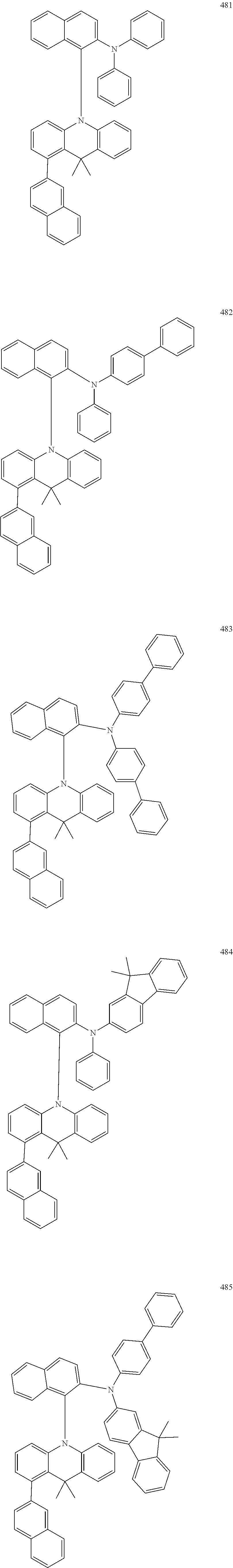 [SMILES: CC1(C)c2ccccc2N(c2c(N(c3ccccc3)c3ccccc3)ccc3ccccc23)c2cccc(-c3ccc4ccccc4c3)c21 ; CC1(C)c2ccccc2N(c2c(N(c3ccccc3)c3ccc(-c4ccccc4)cc3)ccc3ccccc23)c2cccc(-c3ccc4ccccc4c3)c21 ; CC1(C)c2ccccc2N(c2c(N(c3ccc(-c4ccccc4)cc3)c3ccc(-c4ccccc4)cc3)ccc3ccccc23)c2cccc(-c3ccc4ccccc4c3)c21 ; CC1(C)c2ccccc2-c2ccc(N(c3ccccc3)c3ccc4ccccc4c3N3c4ccccc4C(C)(C)c4c(-c5ccc6ccccc6c5)cccc43)cc21 ; CC1(C)c2ccccc2-c2ccc(N(c3ccc(-c4ccccc4)cc3)c3ccc4ccccc4c3N3c4ccccc4C(C)(C)c4c(-c5ccc6ccccc6c5)cccc43)cc21]

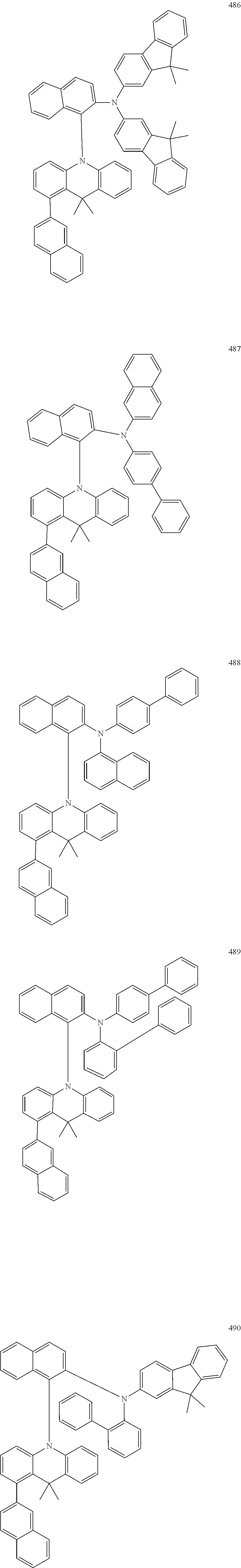

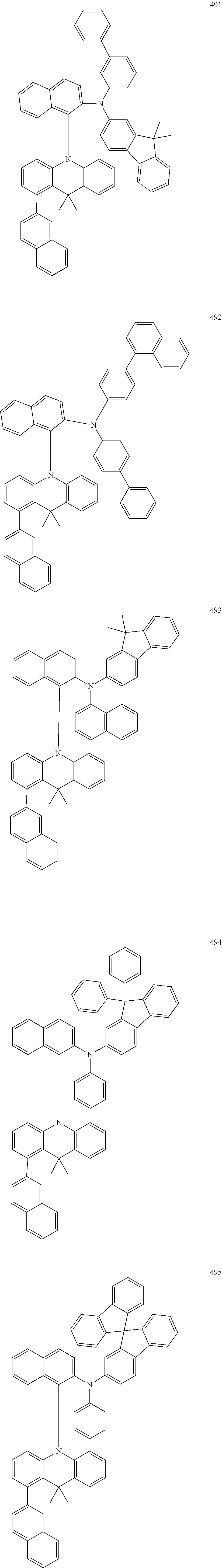

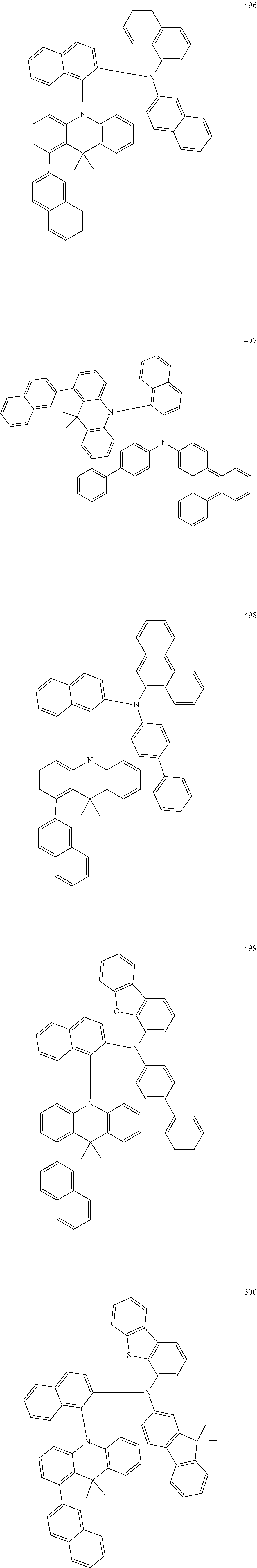

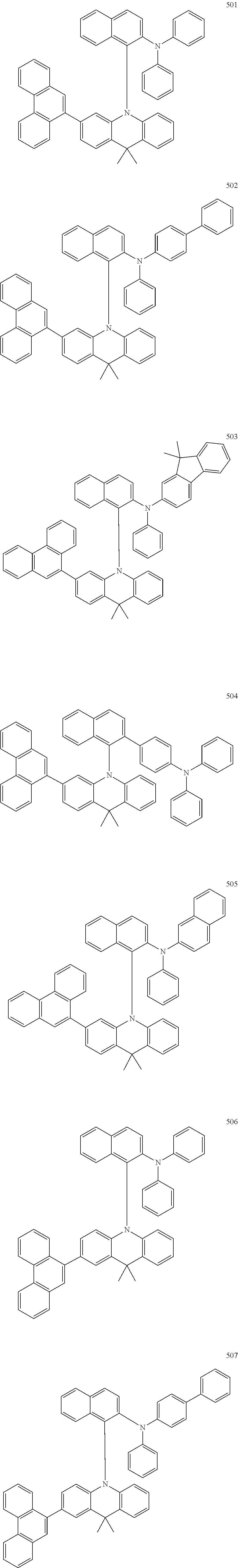

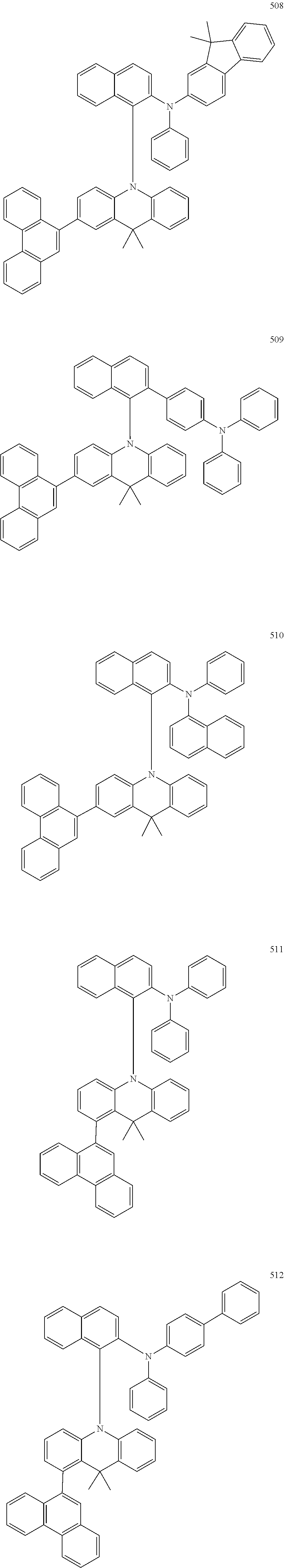 [SMILES: CC1(C)c2ccccc2-c2ccc(N(c3ccccc3)c3ccc4ccccc4c3N3c4ccccc4C(C)(C)c4cc(-c5cc6ccccc6c6ccccc56)ccc43)cc21 ; CC1(C)c2ccccc2N(c2c(-c3ccc(N(c4ccccc4)c4ccccc4)cc3)ccc3ccccc23)c2ccc(-c3cc4ccccc4c4ccccc34)cc21 ; CC1(C)c2ccccc2N(c2c(N(c3ccccc3)c3cccc4ccccc34)ccc3ccccc23)c2ccc(-c3cc4ccccc4c4ccccc34)cc21 ; CC1(C)c2ccccc2N(c2c(N(c3ccccc3)c3ccccc3)ccc3ccccc23)c2cccc(-c3cc4ccccc4c4ccccc34)c21 ; CC1(C)c2ccccc2N(c2c(N(c3ccccc3)c3ccc(-c4ccccc4)cc3)ccc3ccccc23)c2cccc(-c3cc4ccccc4c4ccccc34)c21]

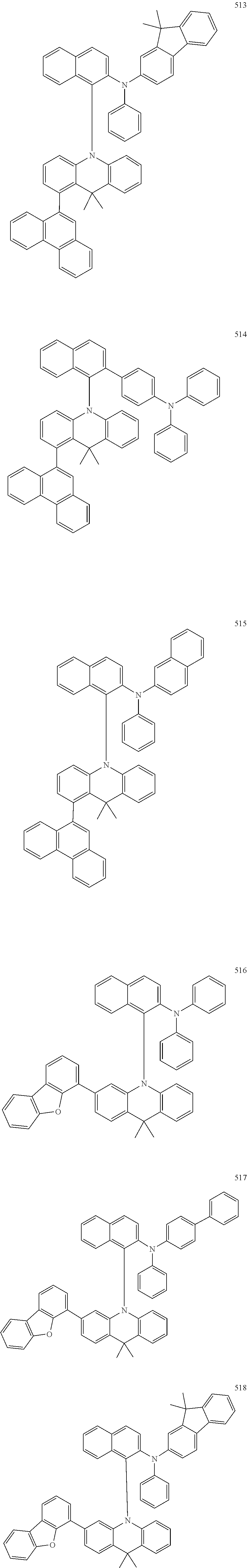

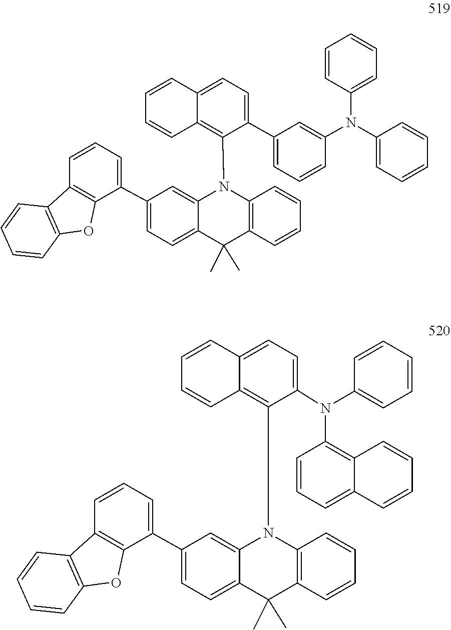 [SMILES: CC1(C)c2ccccc2N(c2c(-c3cccc(N(c4ccccc4)c4ccccc4)c3)ccc3ccccc23)c2cc(-c3cccc4c3oc3ccccc34)ccc21 ; CC1(C)c2ccccc2N(c2c(N(c3ccccc3)c3cccc4ccccc34)ccc3ccccc23)c2cc(-c3cccc4c3oc3ccccc34)ccc21]

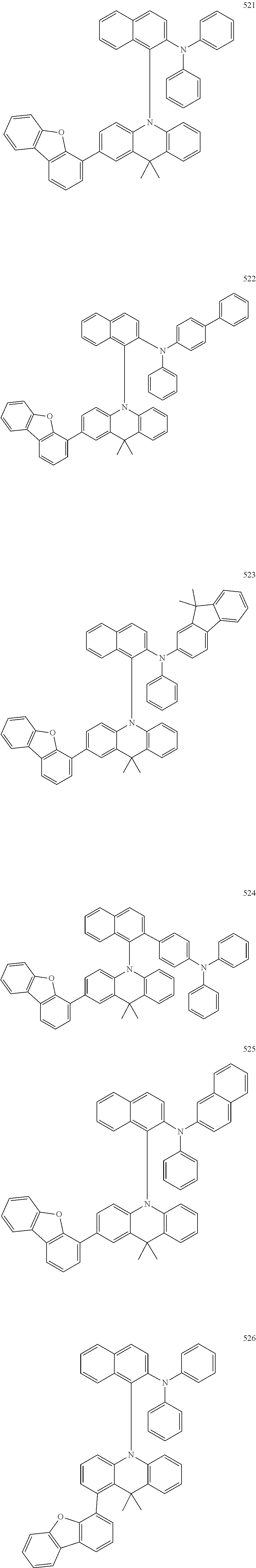

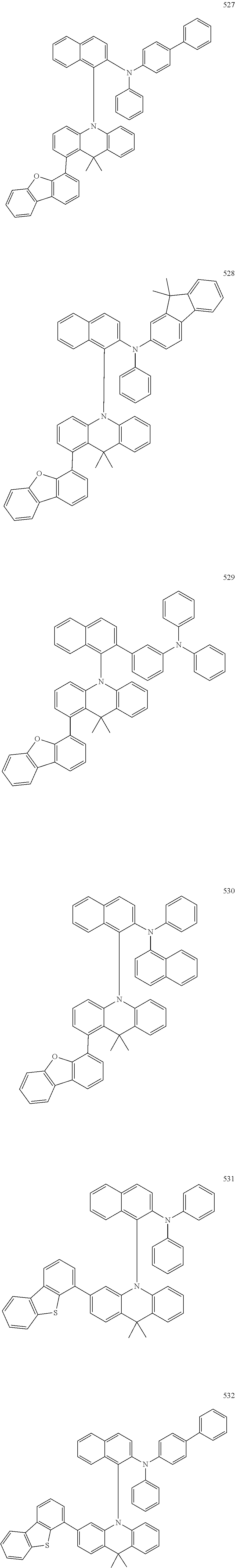

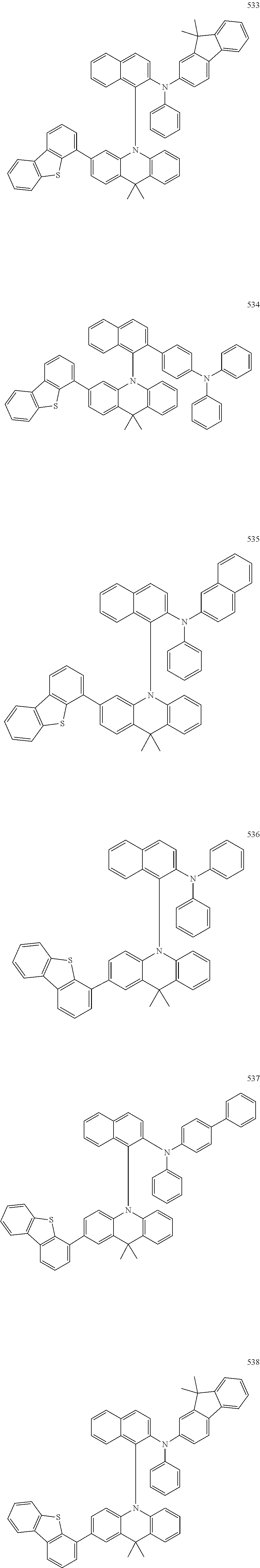

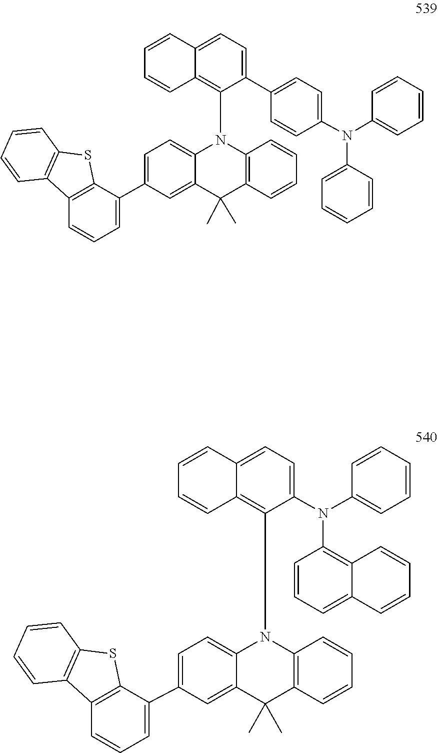 [SMILES: CC1(C)c2ccccc2N(c2c(-c3ccc(N(c4ccccc4)c4ccccc4)cc3)ccc3ccccc23)c2ccc(-c3cccc4c3sc3ccccc34)cc21 ; CC1(C)c2ccccc2N(c2c(N(c3ccccc3)c3cccc4ccccc34)ccc3ccccc23)c2ccc(-c3cccc4c3sc3ccccc34)cc21]

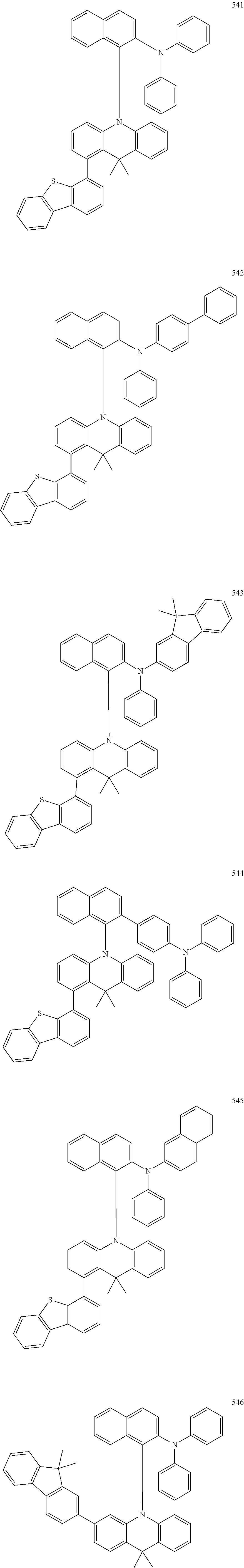

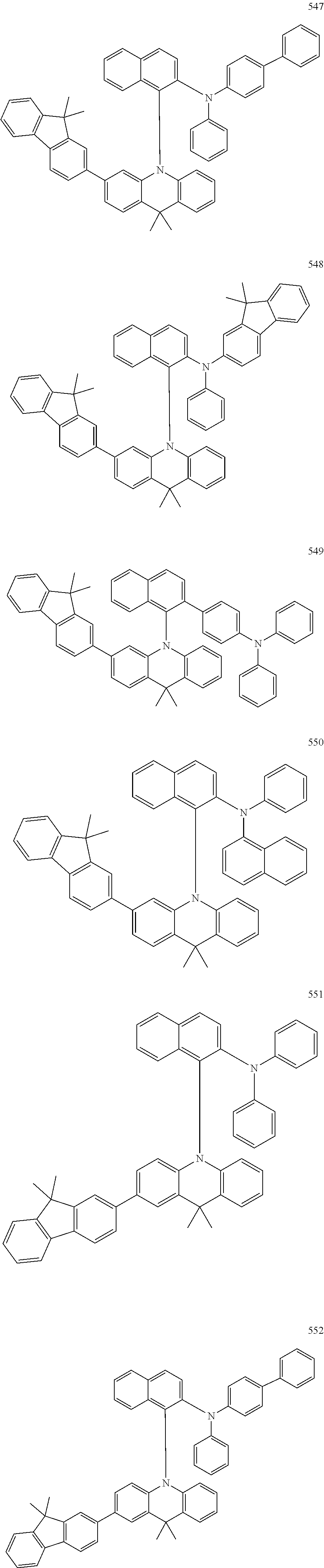

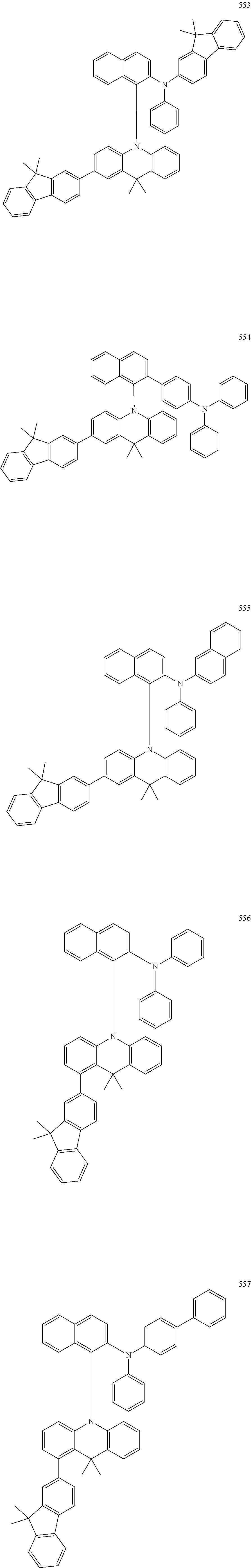

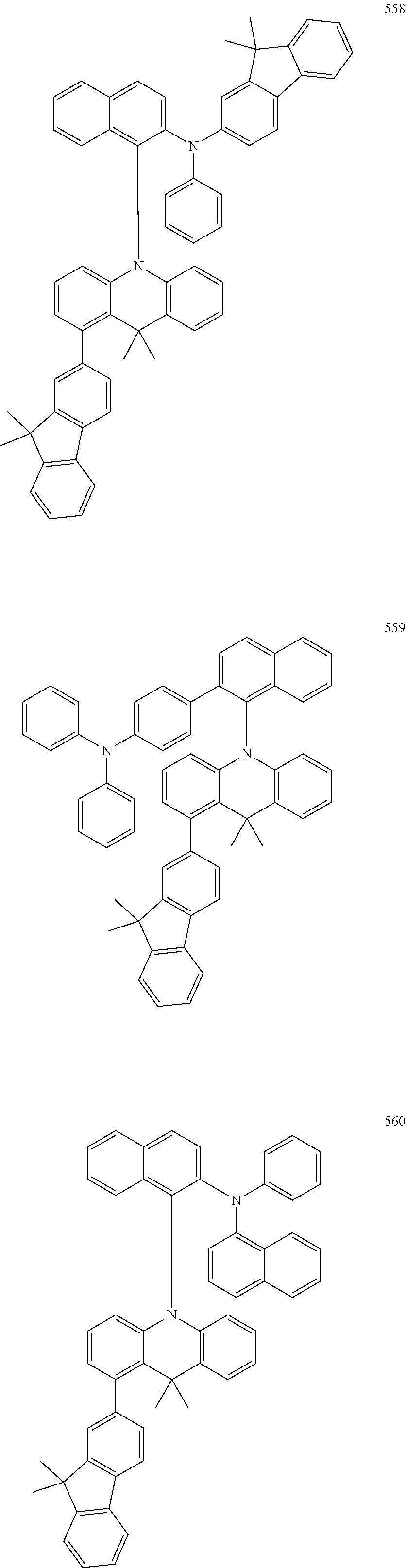 [SMILES: CC1(C)c2ccccc2-c2ccc(-c3cccc4c3C(C)(C)c3ccccc3N4c3c(N(c4ccccc4)c4ccc5c(c4)C(C)(C)c4ccccc4-5)ccc4ccccc34)cc21 ; CC1(C)c2ccccc2-c2ccc(-c3cccc4c3C(C)(C)c3ccccc3N4c3c(-c4ccc(N(c5ccccc5)c5ccccc5)cc4)ccc4ccccc34)cc21 ; CC1(C)c2ccccc2-c2ccc(-c3cccc4c3C(C)(C)c3ccccc3N4c3c(N(c4ccccc4)c4cccc5ccccc45)ccc4ccccc34)cc21]

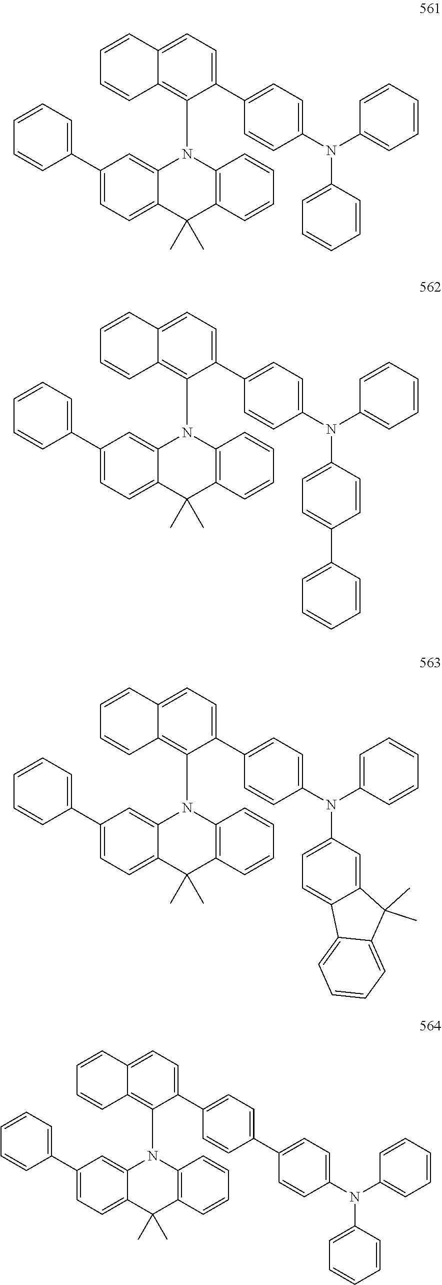 [SMILES: CC1(C)c2ccccc2N(c2c(-c3ccc(N(c4ccccc4)c4ccccc4)cc3)ccc3ccccc23)c2cc(-c3ccccc3)ccc21 ; CC1(C)c2ccccc2N(c2c(-c3ccc(N(c4ccccc4)c4ccc(-c5ccccc5)cc4)cc3)ccc3ccccc23)c2cc(-c3ccccc3)ccc21 ; CC1(C)c2ccccc2-c2ccc(N(c3ccccc3)c3ccc(-c4ccc5ccccc5c4N4c5ccccc5C(C)(C)c5ccc(-c6ccccc6)cc54)cc3)cc21 ; CC1(C)c2ccccc2N(c2c(-c3ccc(-c4ccc(N(c5ccccc5)c5ccccc5)cc4)cc3)ccc3ccccc23)c2cc(-c3ccccc3)ccc21]

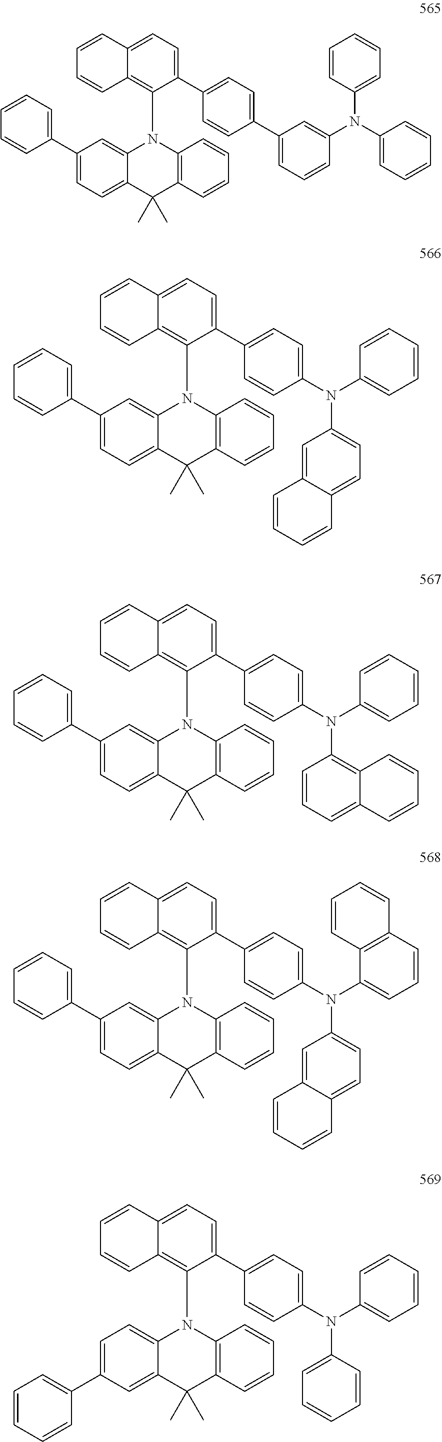 [SMILES: CC1(C)c2ccccc2N(c2c(-c3ccc(-c4cccc(N(c5ccccc5)c5ccccc5)c4)cc3)ccc3ccccc23)c2cc(-c3ccccc3)ccc21 ; CC1(C)c2ccccc2N(c2c(-c3ccc(N(c4ccccc4)c4ccc5ccccc5c4)cc3)ccc3ccccc23)c2cc(-c3ccccc3)ccc21 ; CC1(C)c2ccccc2N(c2c(-c3ccc(N(c4ccccc4)c4cccc5ccccc45)cc3)ccc3ccccc23)c2cc(-c3ccccc3)ccc21 ; CC1(C)c2ccccc2N(c2c(-c3ccc(N(c4ccc5ccccc5c4)c4cccc5ccccc45)cc3)ccc3ccccc23)c2cc(-c3ccccc3)ccc21 ; CC1(C)c2ccccc2N(c2c(-c3ccc(N(c4ccccc4)c4ccccc4)cc3)ccc3ccccc23)c2ccc(-c3ccccc3)cc21]

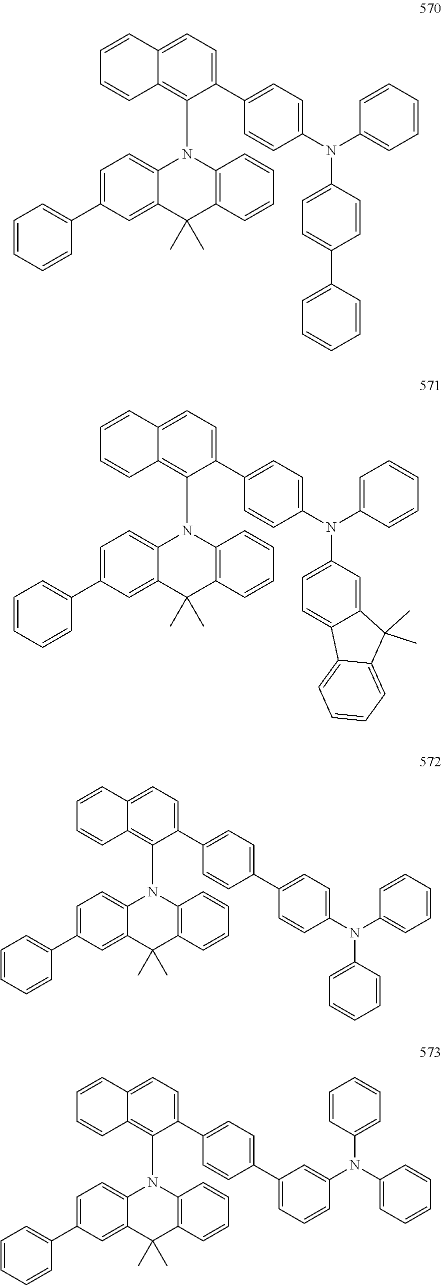 [SMILES: CC1(C)c2ccccc2N(c2c(-c3ccc(N(c4ccccc4)c4ccc(-c5ccccc5)cc4)cc3)ccc3ccccc23)c2ccc(-c3ccccc3)cc21 ; CC1(C)c2ccccc2-c2ccc(N(c3ccccc3)c3ccc(-c4ccc5ccccc5c4N4c5ccccc5C(C)(C)c5cc(-c6ccccc6)ccc54)cc3)cc21 ; CC1(C)c2ccccc2N(c2c(-c3ccc(-c4ccc(N(c5ccccc5)c5ccccc5)cc4)cc3)ccc3ccccc23)c2ccc(-c3ccccc3)cc21 ; CC1(C)c2ccccc2N(c2c(-c3ccc(-c4cccc(N(c5ccccc5)c5ccccc5)c4)cc3)ccc3ccccc23)c2ccc(-c3ccccc3)cc21]

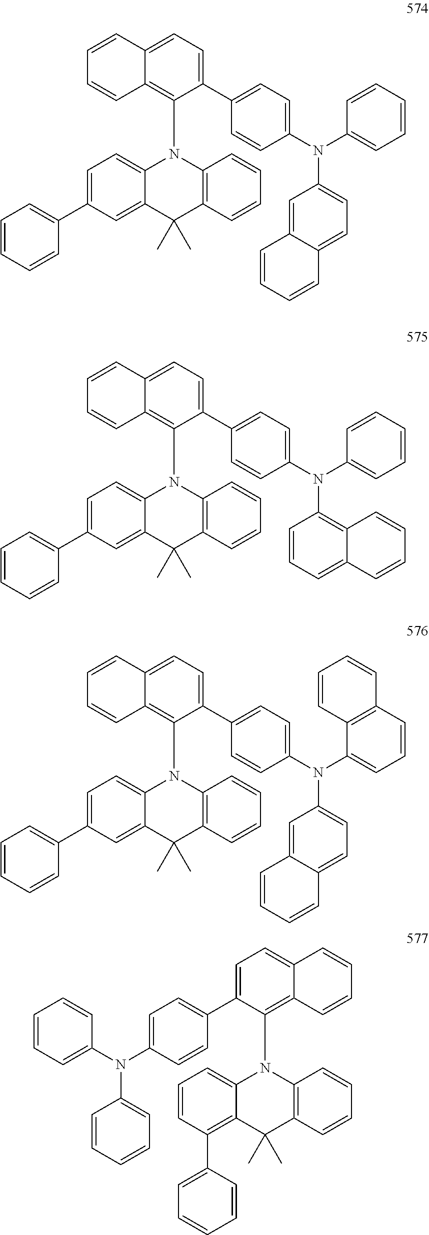

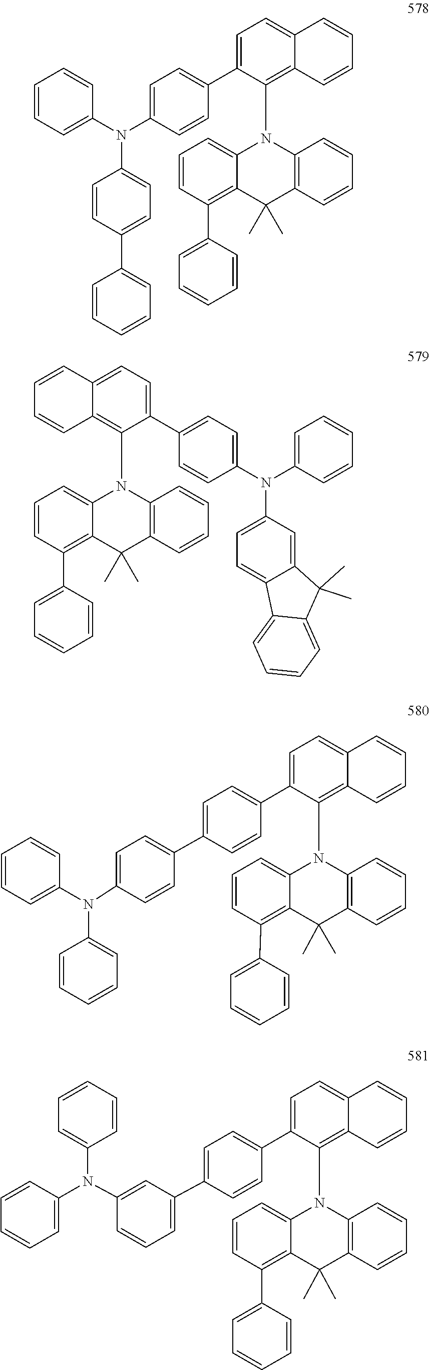

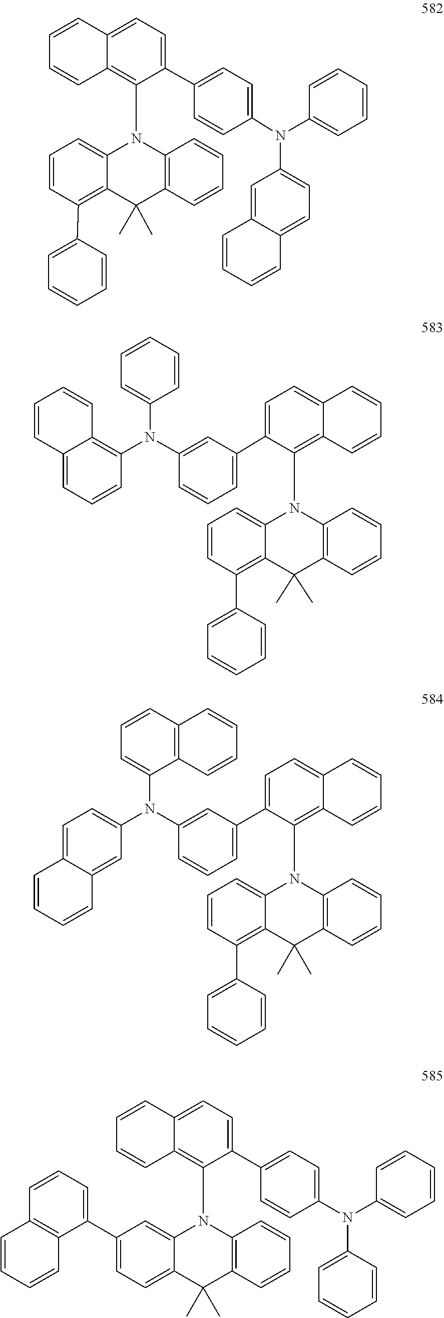

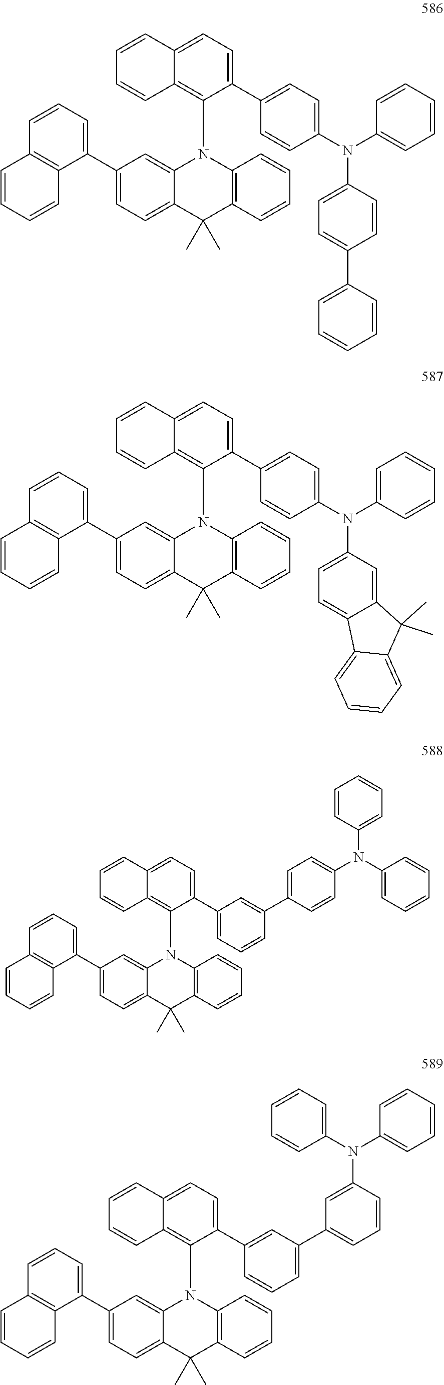 [SMILES: CC1(C)c2ccccc2N(c2c(-c3ccc(N(c4ccccc4)c4ccc(-c5ccccc5)cc4)cc3)ccc3ccccc23)c2cc(-c3cccc4ccccc34)ccc21 ; CC1(C)c2ccccc2-c2ccc(N(c3ccccc3)c3ccc(-c4ccc5ccccc5c4N4c5ccccc5C(C)(C)c5ccc(-c6cccc7ccccc67)cc54)cc3)cc21 ; CC1(C)c2ccccc2N(c2c(-c3cccc(-c4ccc(N(c5ccccc5)c5ccccc5)cc4)c3)ccc3ccccc23)c2cc(-c3cccc4ccccc34)ccc21 ; CC1(C)c2ccccc2N(c2c(-c3cccc(-c4cccc(N(c5ccccc5)c5ccccc5)c4)c3)ccc3ccccc23)c2cc(-c3cccc4ccccc34)ccc21]

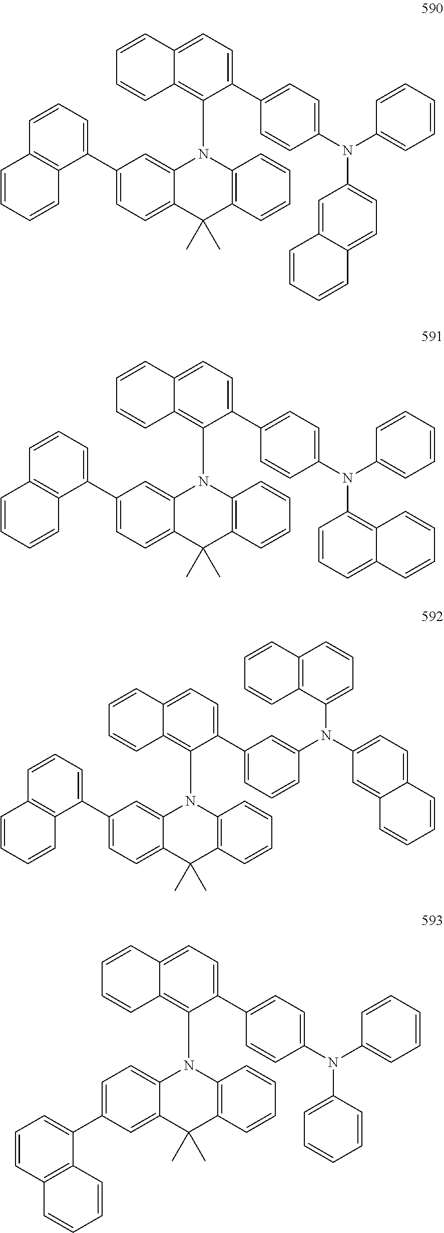 [SMILES: CC1(C)c2ccccc2N(c2c(-c3ccc(N(c4ccccc4)c4ccc5ccccc5c4)cc3)ccc3ccccc23)c2cc(-c3cccc4ccccc34)ccc21 ; CC1(C)c2ccccc2N(c2c(-c3ccc(N(c4ccccc4)c4cccc5ccccc45)cc3)ccc3ccccc23)c2cc(-c3cccc4ccccc34)ccc21 ; CC1(C)c2ccccc2N(c2c(-c3cccc(N(c4ccc5ccccc5c4)c4cccc5ccccc45)c3)ccc3ccccc23)c2cc(-c3cccc4ccccc34)ccc21 ; CC1(C)c2ccccc2N(c2c(-c3ccc(N(c4ccccc4)c4ccccc4)cc3)ccc3ccccc23)c2ccc(-c3cccc4ccccc34)cc21]

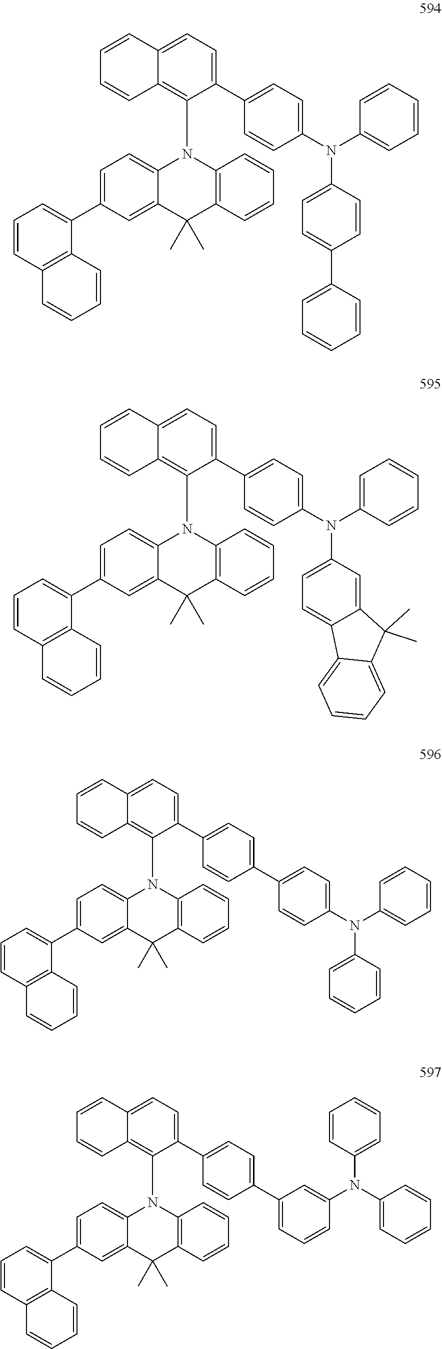 [SMILES: CC1(C)c2ccccc2N(c2c(-c3ccc(N(c4ccccc4)c4ccc(-c5ccccc5)cc4)cc3)ccc3ccccc23)c2ccc(-c3cccc4ccccc34)cc21 ; CC1(C)c2ccccc2-c2ccc(N(c3ccccc3)c3ccc(-c4ccc5ccccc5c4N4c5ccccc5C(C)(C)c5cc(-c6cccc7ccccc67)ccc54)cc3)cc21 ; CC1(C)c2ccccc2N(c2c(-c3ccc(-c4ccc(N(c5ccccc5)c5ccccc5)cc4)cc3)ccc3ccccc23)c2ccc(-c3cccc4ccccc34)cc21 ; CC1(C)c2ccccc2N(c2c(-c3ccc(-c4cccc(N(c5ccccc5)c5ccccc5)c4)cc3)ccc3ccccc23)c2ccc(-c3cccc4ccccc34)cc21]

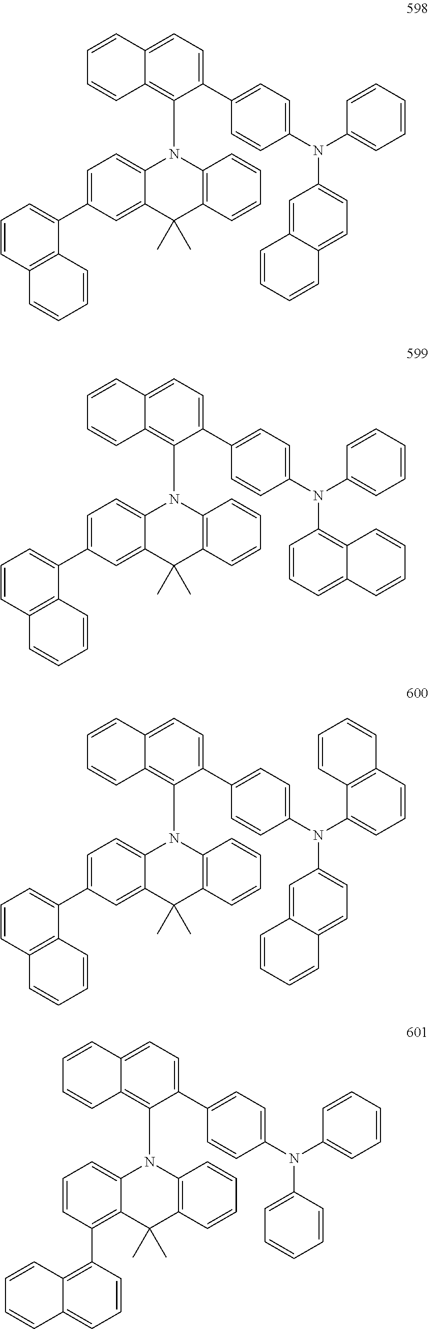 [SMILES: CC1(C)c2ccccc2N(c2c(-c3ccc(N(c4ccccc4)c4ccc5ccccc5c4)cc3)ccc3ccccc23)c2ccc(-c3cccc4ccccc34)cc21 ; CC1(C)c2ccccc2N(c2c(-c3ccc(N(c4ccccc4)c4cccc5ccccc45)cc3)ccc3ccccc23)c2ccc(-c3cccc4ccccc34)cc21 ; CC1(C)c2ccccc2N(c2c(-c3ccc(N(c4ccc5ccccc5c4)c4cccc5ccccc45)cc3)ccc3ccccc23)c2ccc(-c3cccc4ccccc34)cc21 ; CC1(C)c2ccccc2N(c2c(-c3ccc(N(c4ccccc4)c4ccccc4)cc3)ccc3ccccc23)c2cccc(-c3cccc4ccccc34)c21]

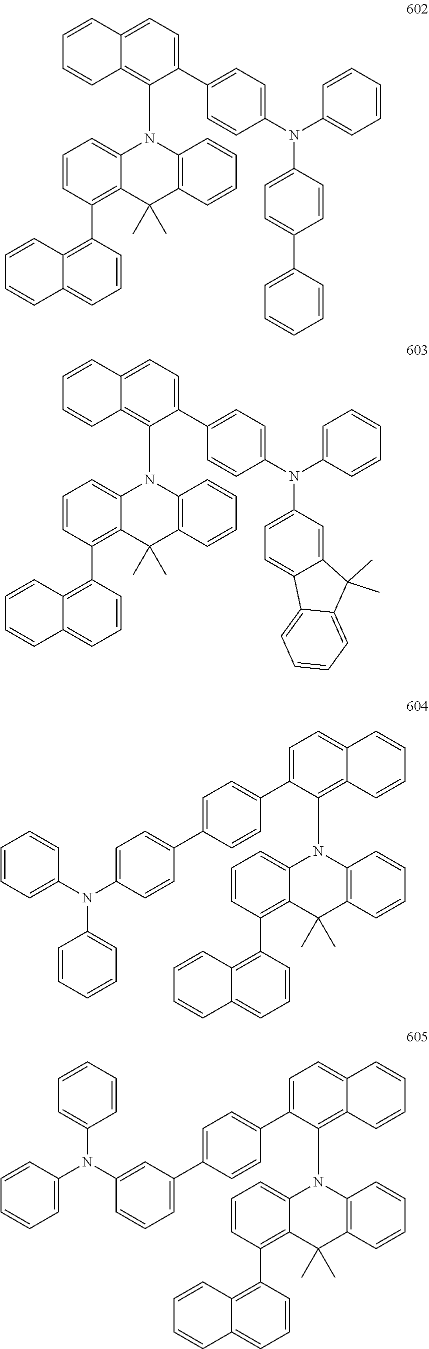 [SMILES: CC1(C)c2ccccc2N(c2c(-c3ccc(N(c4ccccc4)c4ccc(-c5ccccc5)cc4)cc3)ccc3ccccc23)c2cccc(-c3cccc4ccccc34)c21 ; CC1(C)c2ccccc2-c2ccc(N(c3ccccc3)c3ccc(-c4ccc5ccccc5c4N4c5ccccc5C(C)(C)c5c(-c6cccc7ccccc67)cccc54)cc3)cc21 ; CC1(C)c2ccccc2N(c2c(-c3ccc(-c4ccc(N(c5ccccc5)c5ccccc5)cc4)cc3)ccc3ccccc23)c2cccc(-c3cccc4ccccc34)c21 ; CC1(C)c2ccccc2N(c2c(-c3ccc(-c4cccc(N(c5ccccc5)c5ccccc5)c4)cc3)ccc3ccccc23)c2cccc(-c3cccc4ccccc34)c21]

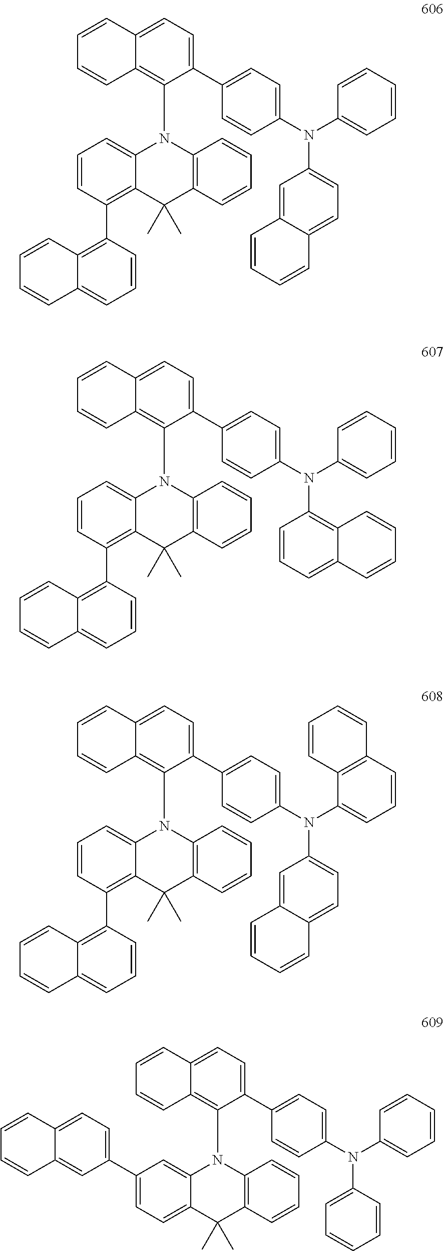 [SMILES: CC1(C)c2ccccc2N(c2c(-c3ccc(N(c4ccccc4)c4ccc5ccccc5c4)cc3)ccc3ccccc23)c2cccc(-c3cccc4ccccc34)c21 ; CC1(C)c2ccccc2N(c2c(-c3ccc(N(c4ccccc4)c4cccc5ccccc45)cc3)ccc3ccccc23)c2cccc(-c3cccc4ccccc34)c21 ; CC1(C)c2ccccc2N(c2c(-c3ccc(N(c4ccc5ccccc5c4)c4cccc5ccccc45)cc3)ccc3ccccc23)c2cccc(-c3cccc4ccccc34)c21 ; CC1(C)c2ccccc2N(c2c(-c3ccc(N(c4ccccc4)c4ccccc4)cc3)ccc3ccccc23)c2cc(-c3ccc4ccccc4c3)ccc21]

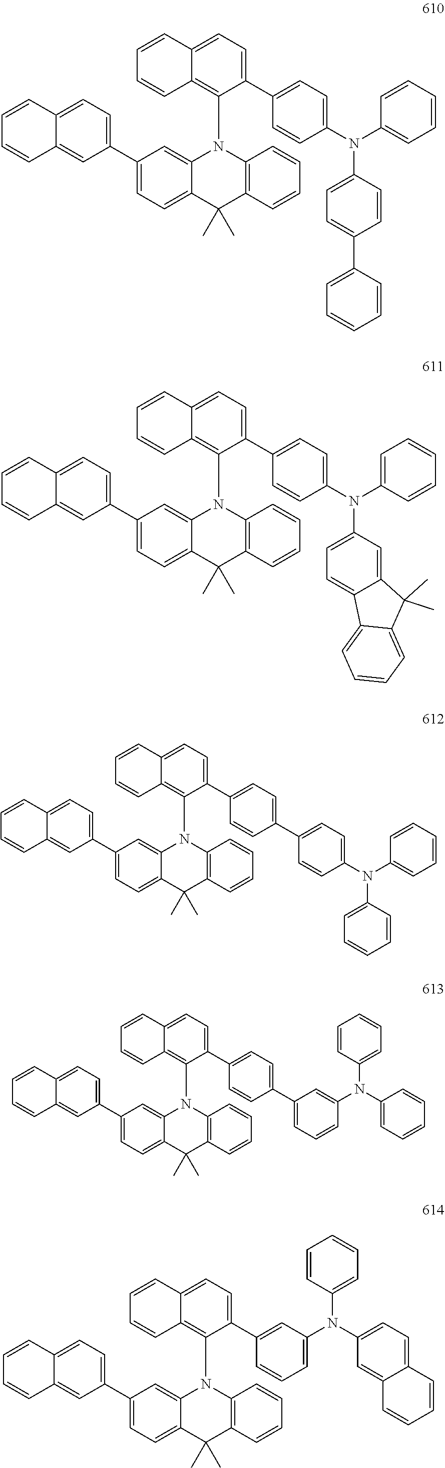

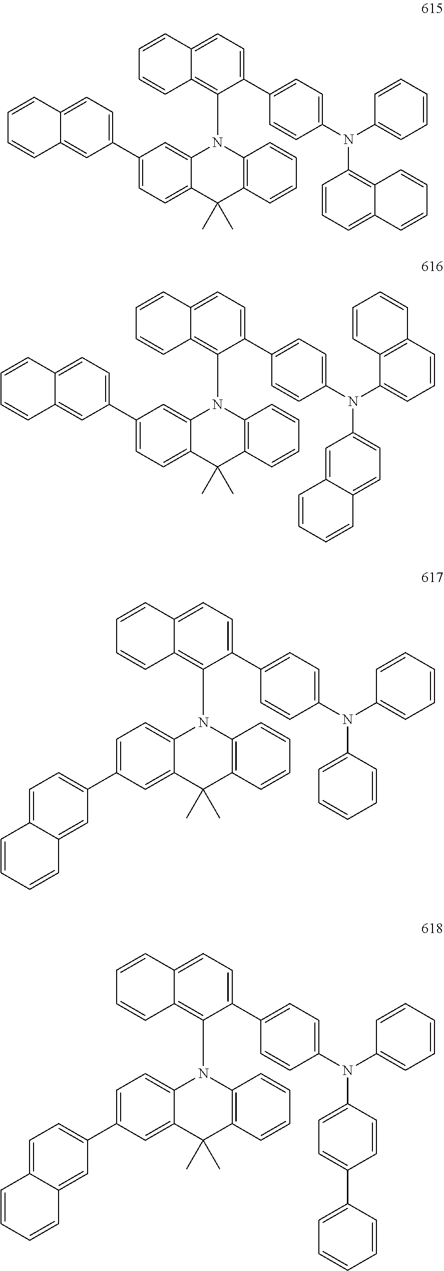 [SMILES: CC1(C)c2ccccc2N(c2c(-c3ccc(N(c4ccccc4)c4cccc5ccccc45)cc3)ccc3ccccc23)c2cc(-c3ccc4ccccc4c3)ccc21 ; CC1(C)c2ccccc2N(c2c(-c3ccc(N(c4ccc5ccccc5c4)c4cccc5ccccc45)cc3)ccc3ccccc23)c2cc(-c3ccc4ccccc4c3)ccc21 ; CC1(C)c2ccccc2N(c2c(-c3ccc(N(c4ccccc4)c4ccccc4)cc3)ccc3ccccc23)c2ccc(-c3ccc4ccccc4c3)cc21 ; CC1(C)c2ccccc2N(c2c(-c3ccc(N(c4ccccc4)c4ccc(-c5ccccc5)cc4)cc3)ccc3ccccc23)c2ccc(-c3ccc4ccccc4c3)cc21]

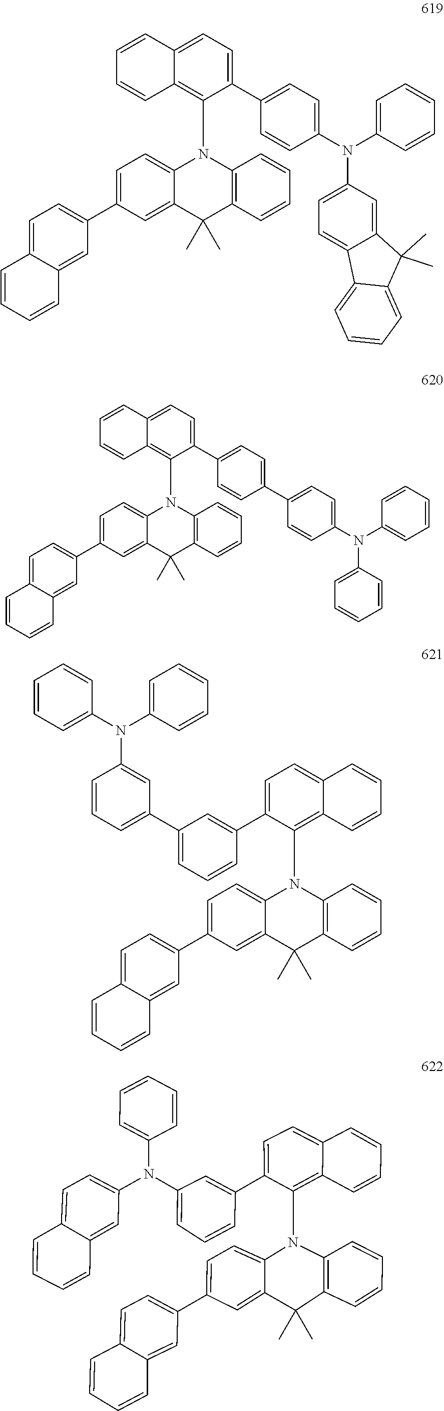

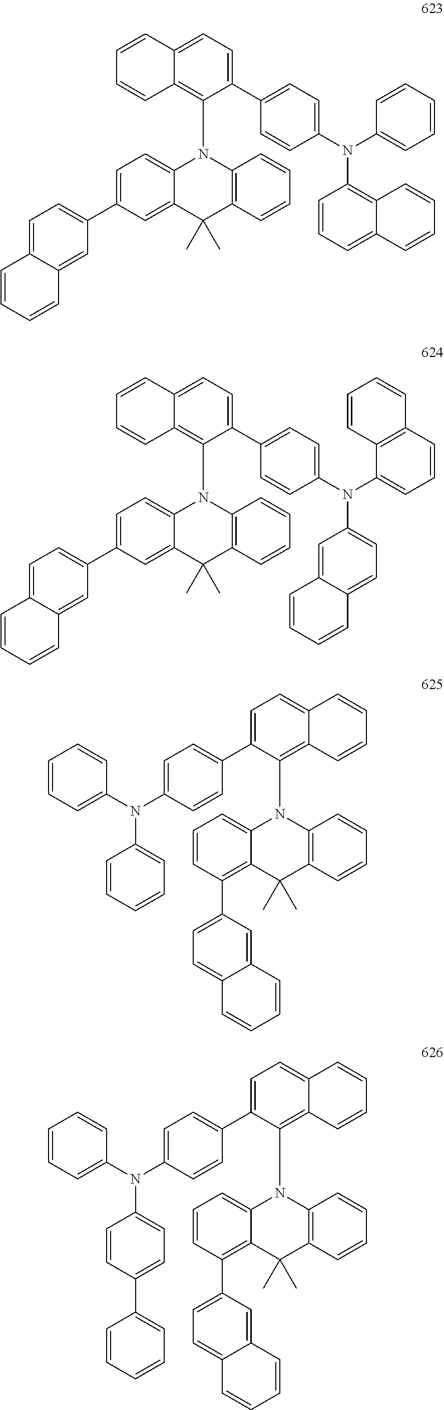

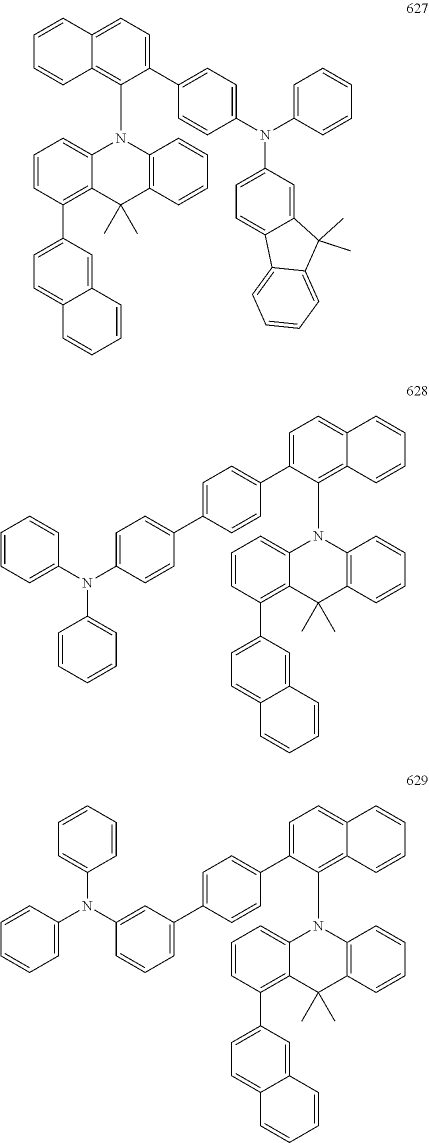 [SMILES: CC1(C)c2ccccc2-c2ccc(N(c3ccccc3)c3ccc(-c4ccc5ccccc5c4N4c5ccccc5C(C)(C)c5c(-c6ccc7ccccc7c6)cccc54)cc3)cc21 ; CC1(C)c2ccccc2N(c2c(-c3ccc(-c4ccc(N(c5ccccc5)c5ccccc5)cc4)cc3)ccc3ccccc23)c2cccc(-c3ccc4ccccc4c3)c21 ; CC1(C)c2ccccc2N(c2c(-c3ccc(-c4cccc(N(c5ccccc5)c5ccccc5)c4)cc3)ccc3ccccc23)c2cccc(-c3ccc4ccccc4c3)c21]

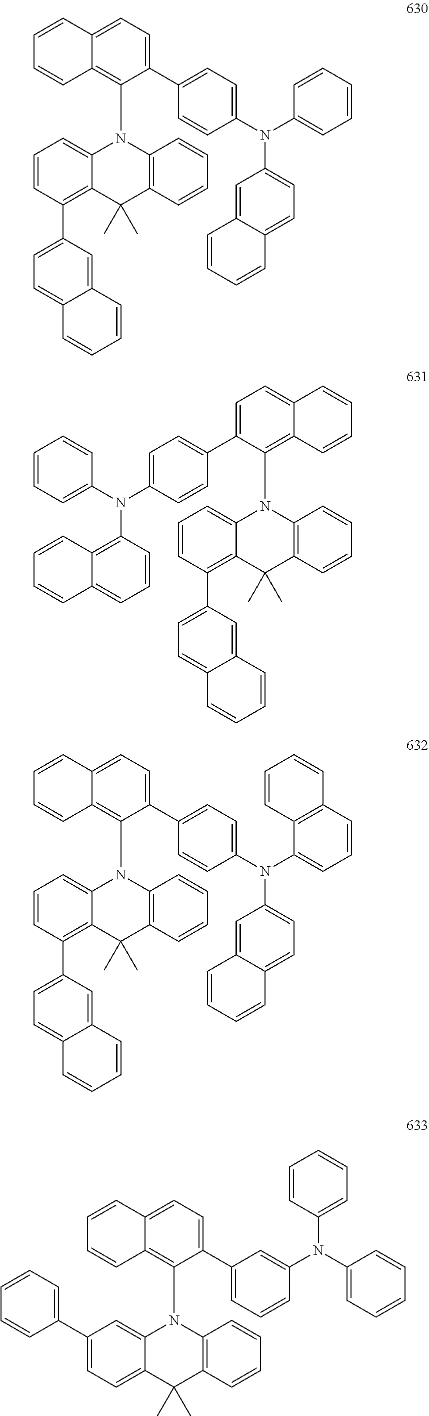 [SMILES: CC1(C)c2ccccc2N(c2c(-c3ccc(N(c4ccccc4)c4ccc5ccccc5c4)cc3)ccc3ccccc23)c2cccc(-c3ccc4ccccc4c3)c21 ; CC1(C)c2ccccc2N(c2c(-c3ccc(N(c4ccccc4)c4cccc5ccccc45)cc3)ccc3ccccc23)c2cccc(-c3ccc4ccccc4c3)c21 ; CC1(C)c2ccccc2N(c2c(-c3ccc(N(c4ccc5ccccc5c4)c4cccc5ccccc45)cc3)ccc3ccccc23)c2cccc(-c3ccc4ccccc4c3)c21 ; CC1(C)c2ccccc2N(c2c(-c3cccc(N(c4ccccc4)c4ccccc4)c3)ccc3ccccc23)c2cc(-c3ccccc3)ccc21]

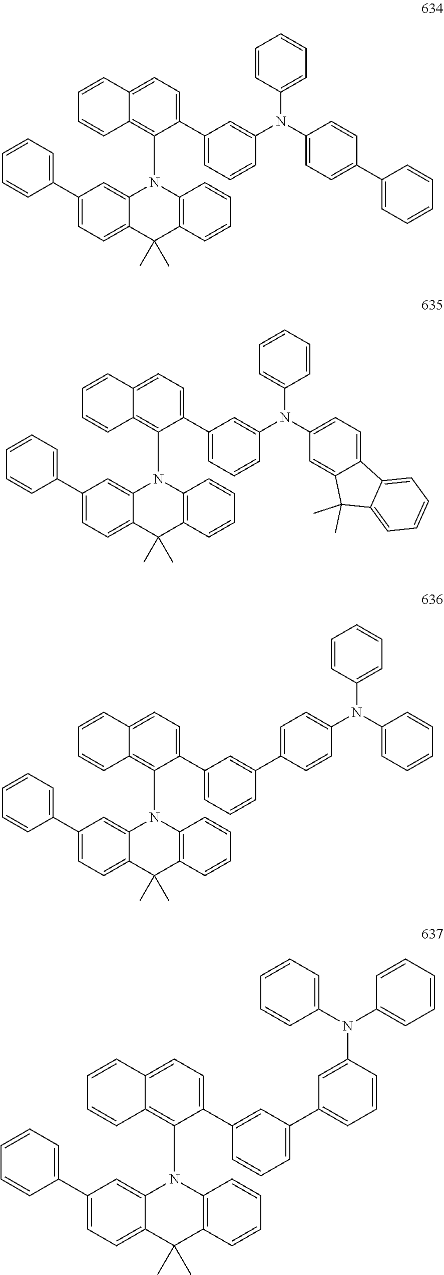 [SMILES: CC1(C)c2ccccc2N(c2c(-c3cccc(N(c4ccccc4)c4ccc(-c5ccccc5)cc4)c3)ccc3ccccc23)c2cc(-c3ccccc3)ccc21 ; CC1(C)c2ccccc2-c2ccc(N(c3ccccc3)c3cccc(-c4ccc5ccccc5c4N4c5ccccc5C(C)(C)c5ccc(-c6ccccc6)cc54)c3)cc21 ; CC1(C)c2ccccc2N(c2c(-c3cccc(-c4ccc(N(c5ccccc5)c5ccccc5)cc4)c3)ccc3ccccc23)c2cc(-c3ccccc3)ccc21 ; CC1(C)c2ccccc2N(c2c(-c3cccc(-c4cccc(N(c5ccccc5)c5ccccc5)c4)c3)ccc3ccccc23)c2cc(-c3ccccc3)ccc21]

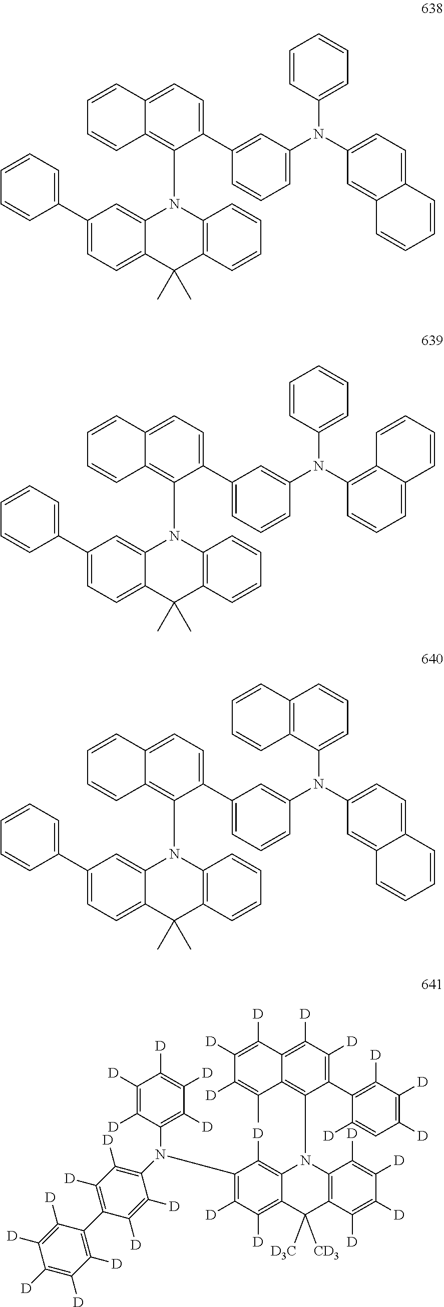

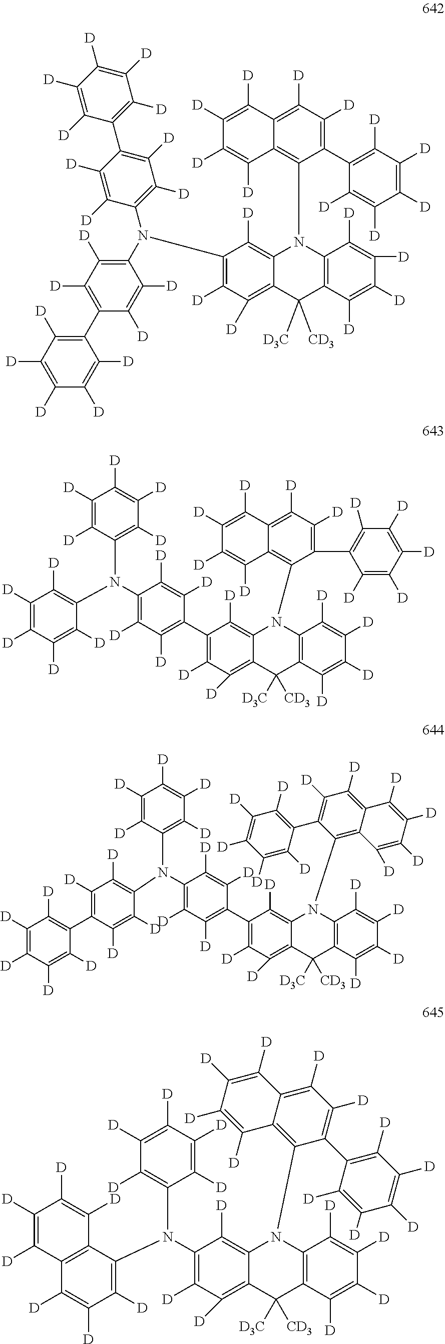

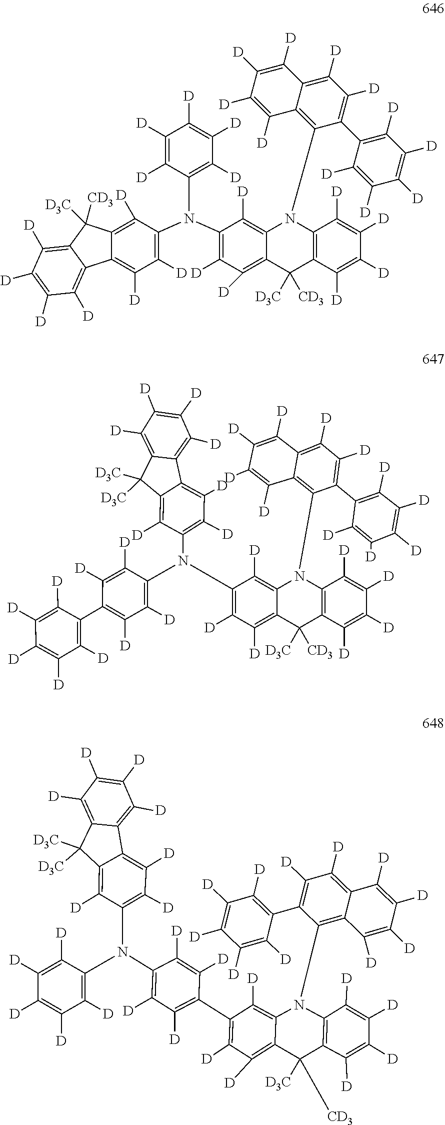

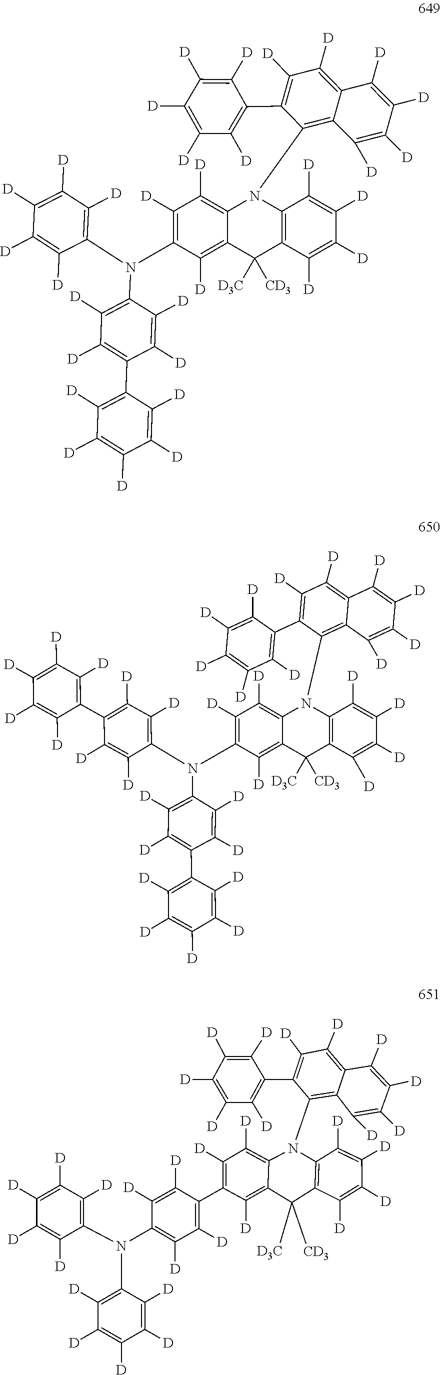

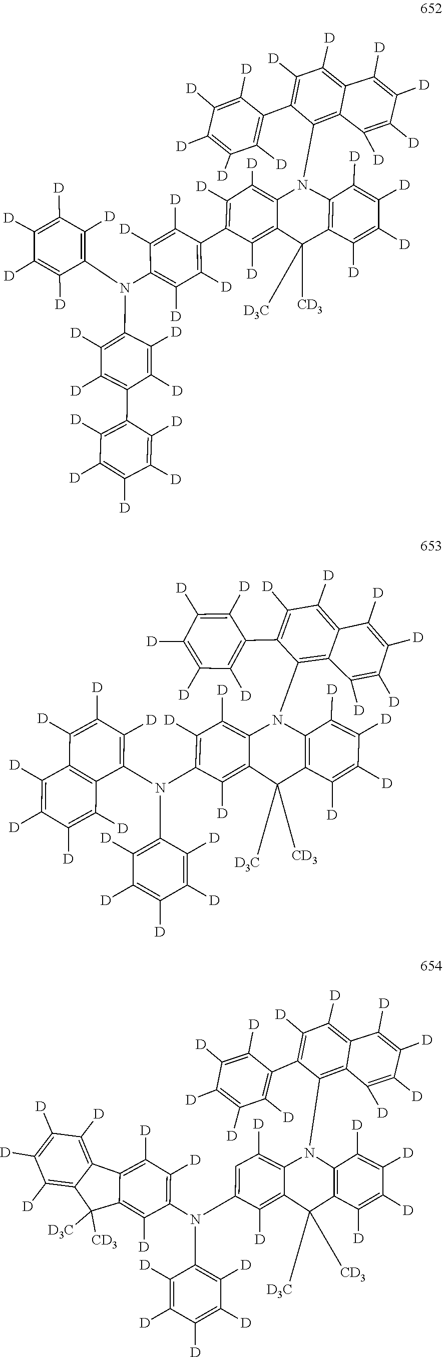

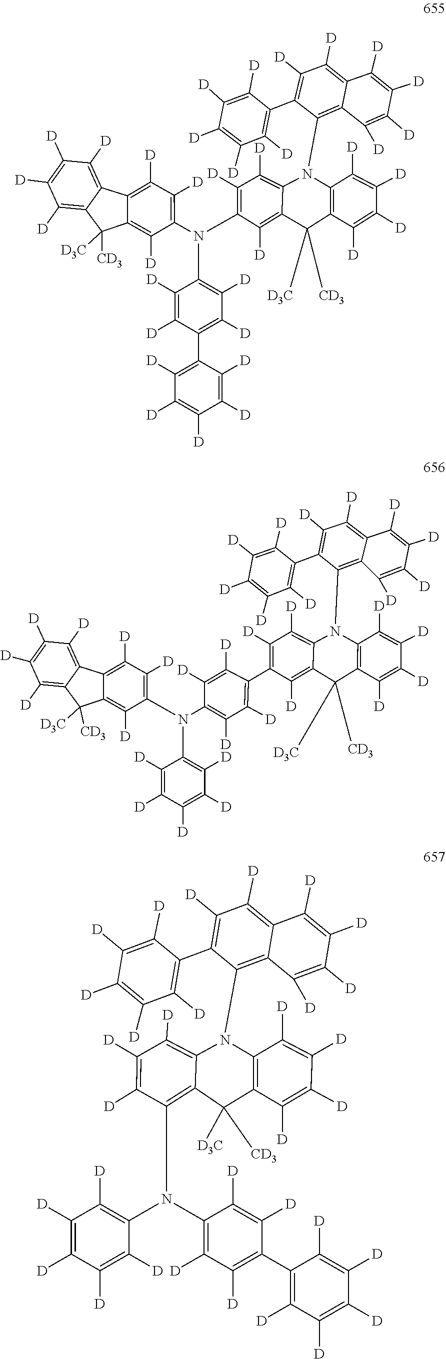

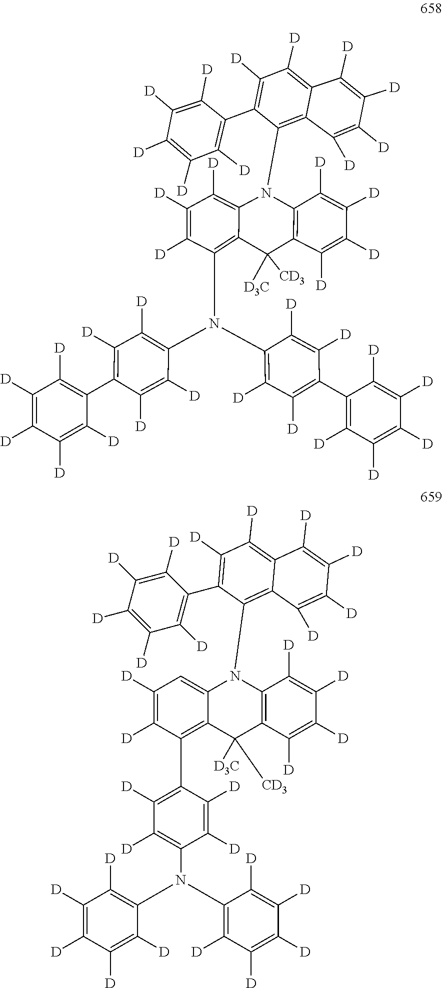

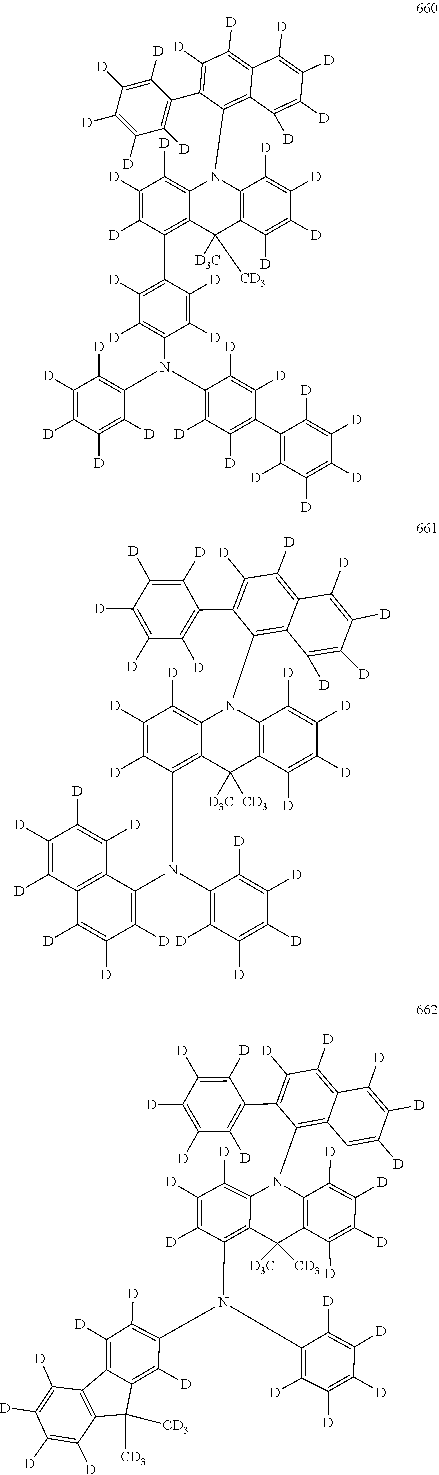

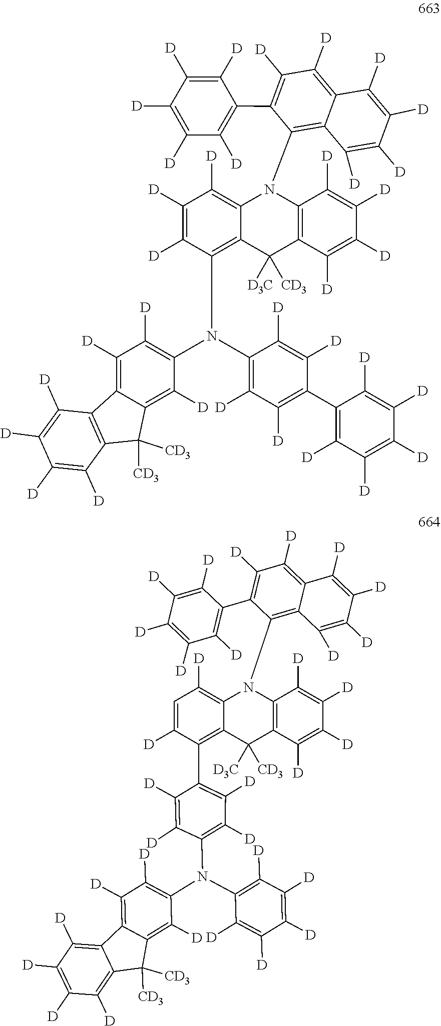

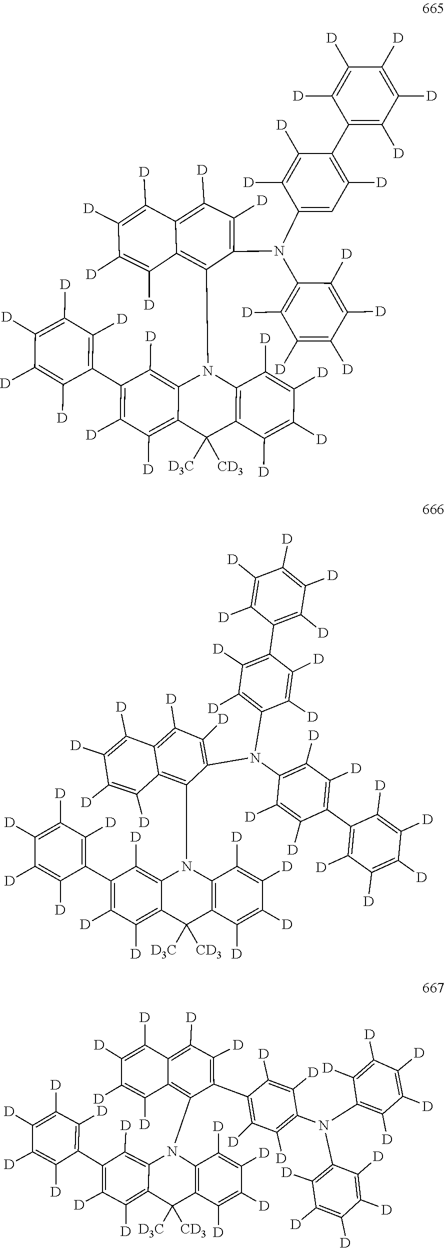

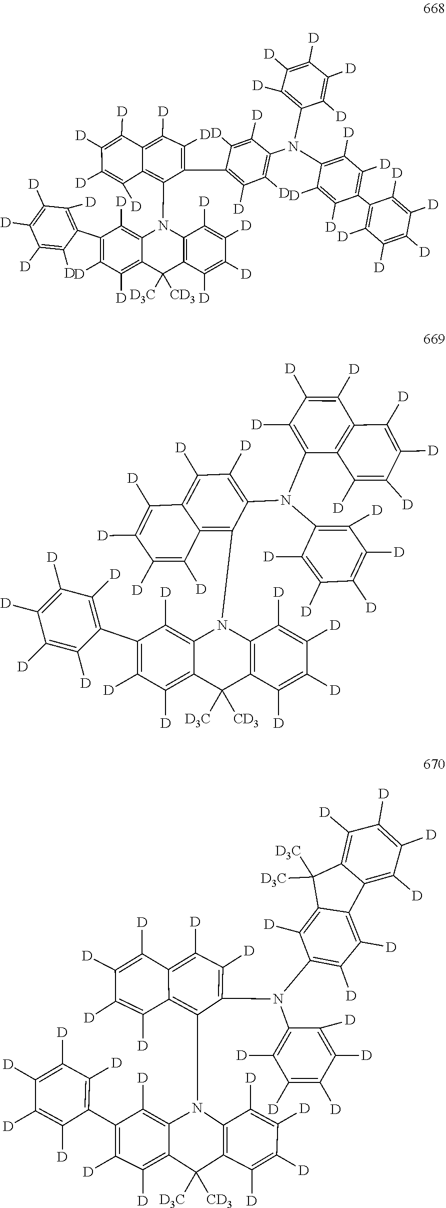

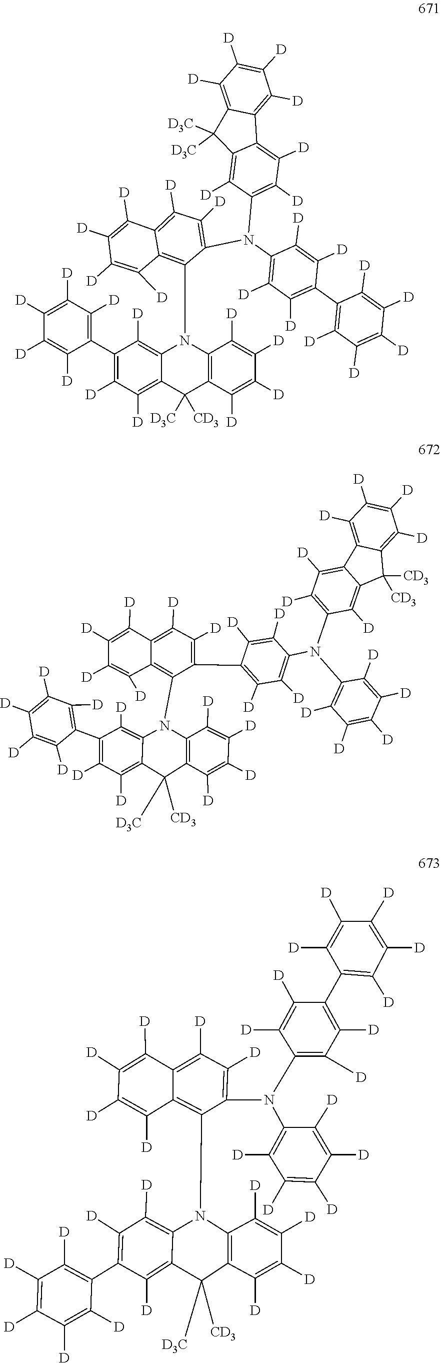

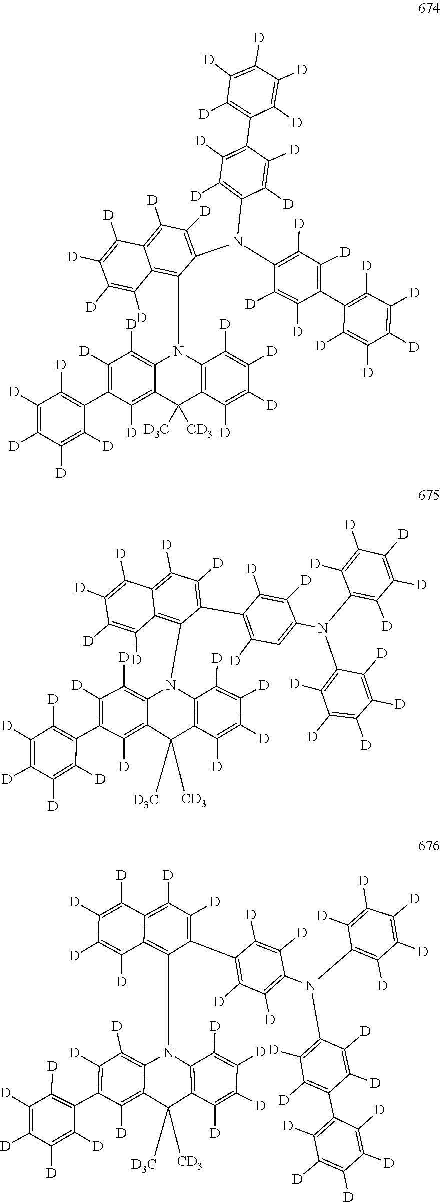

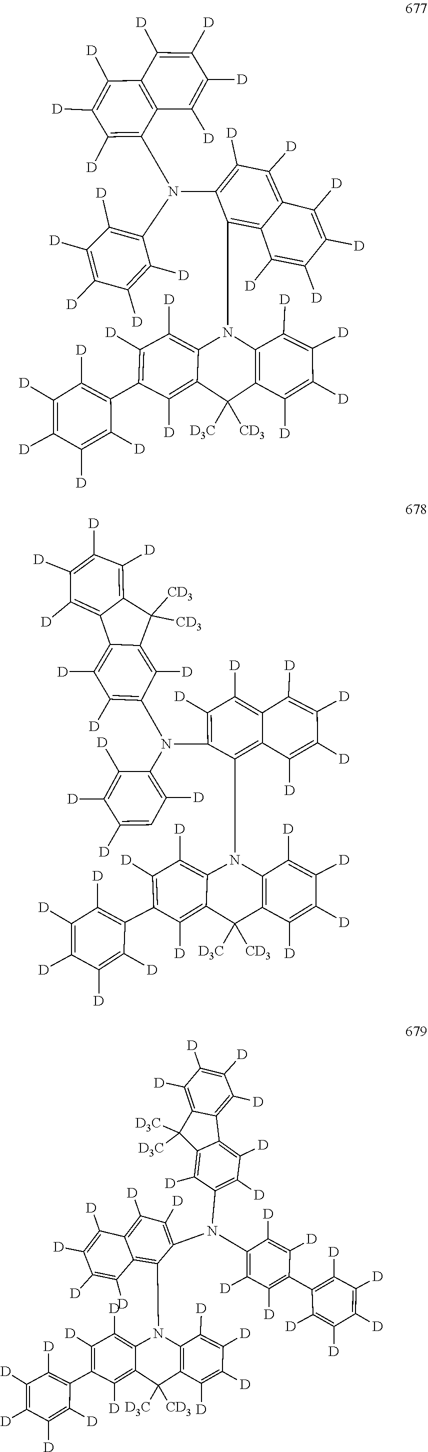

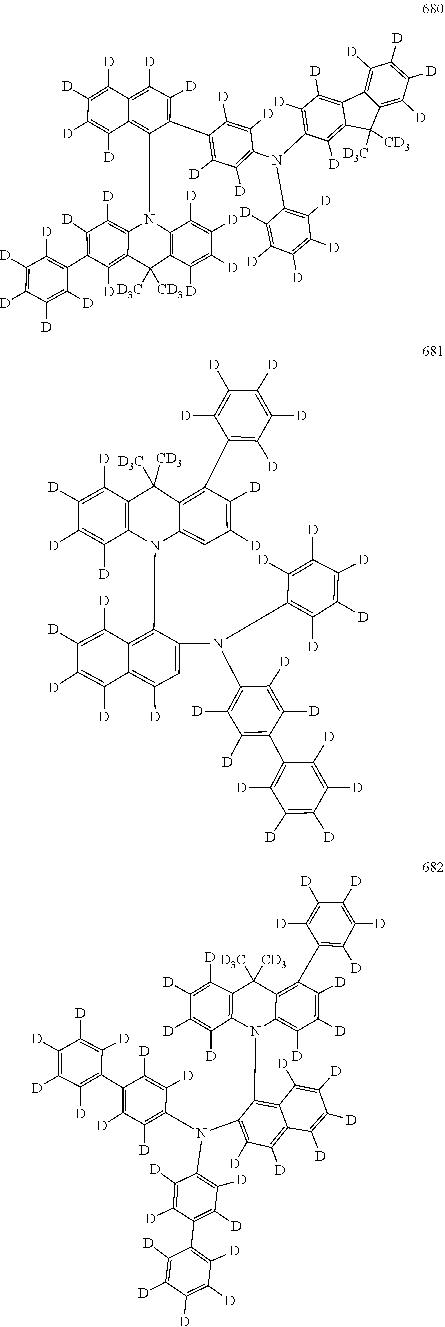

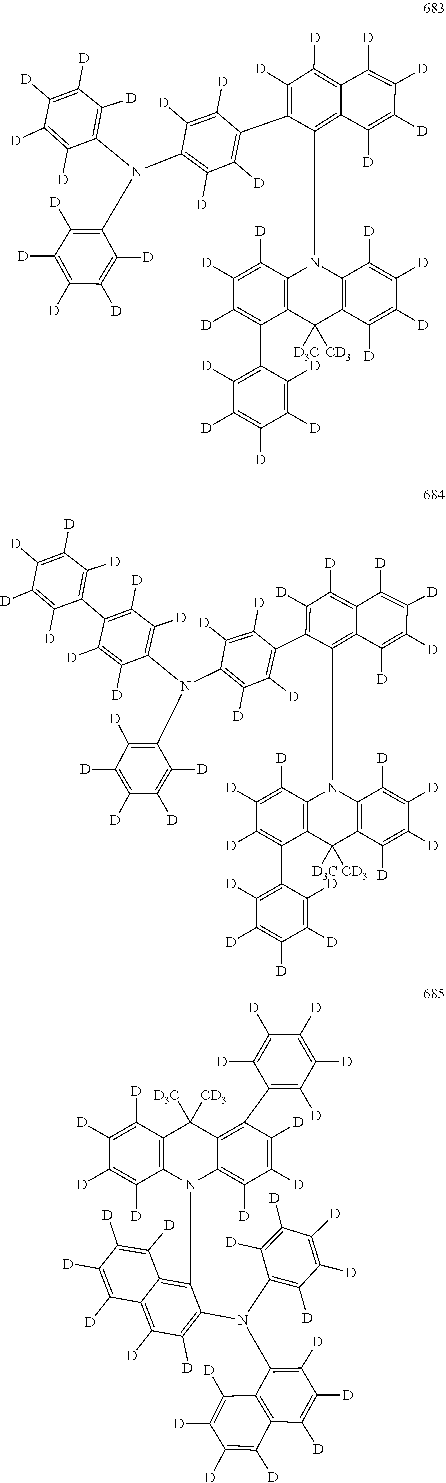

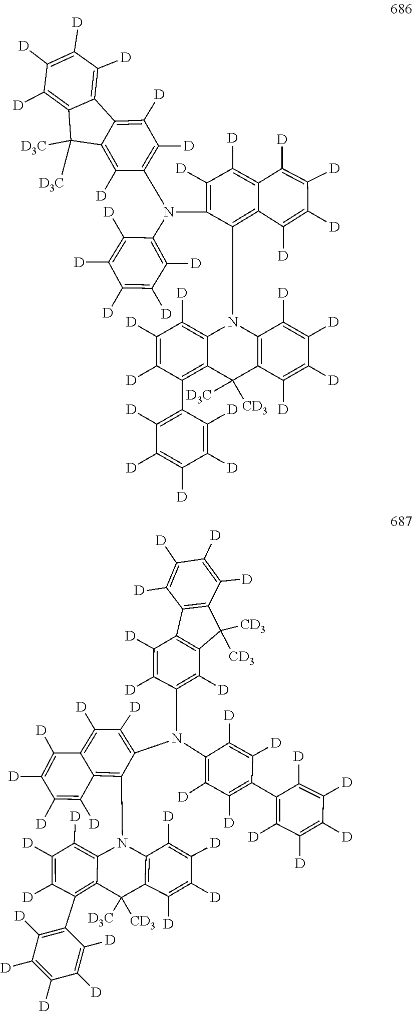

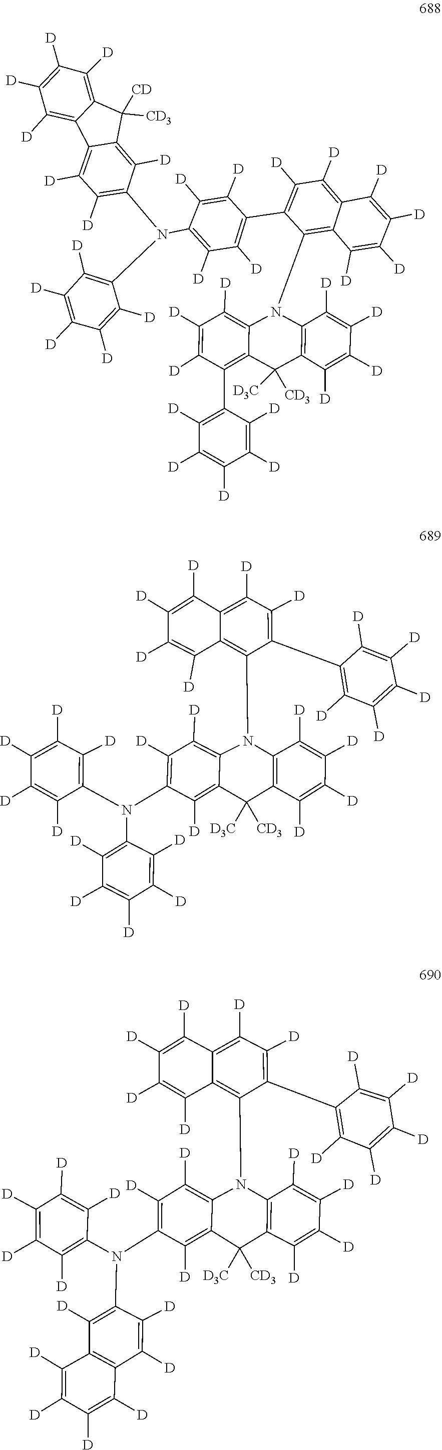

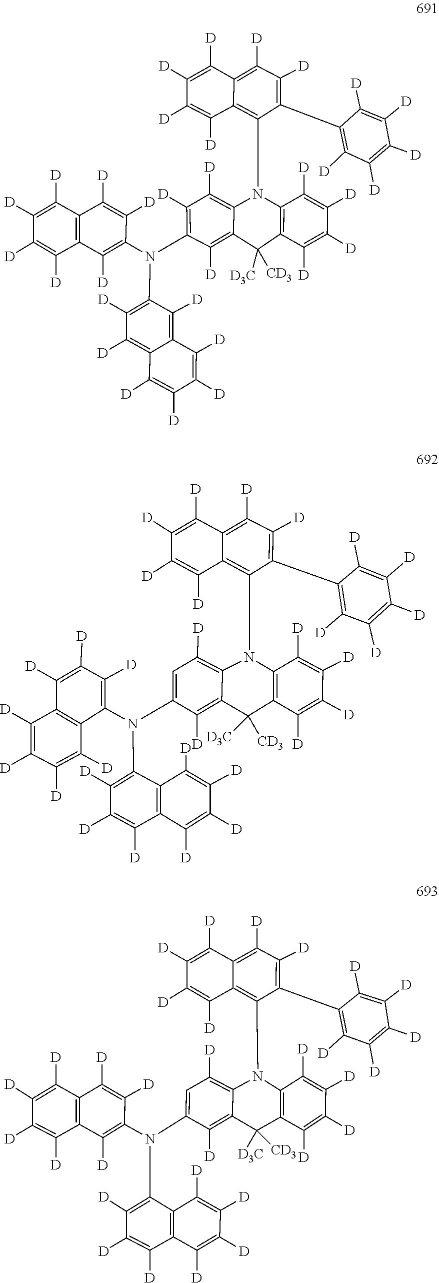

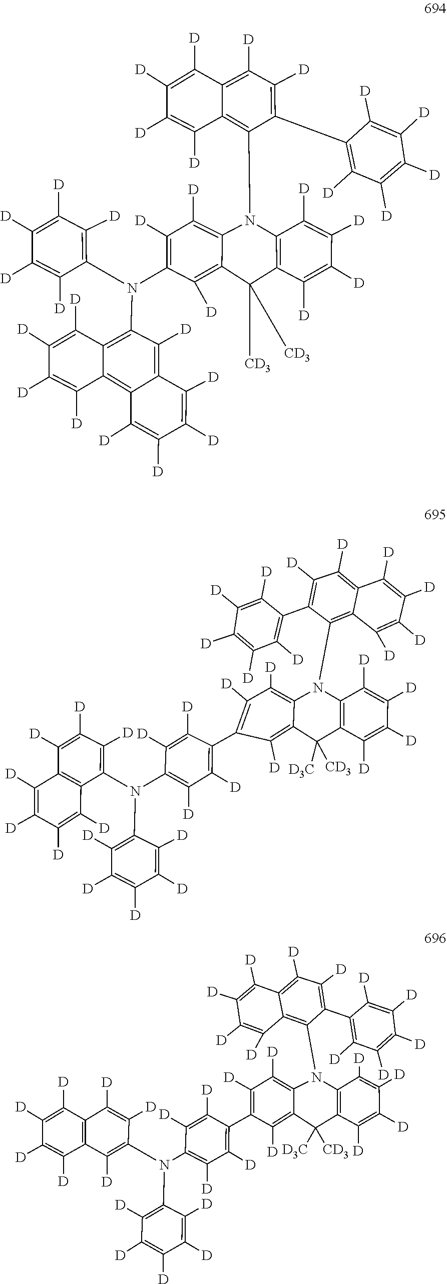

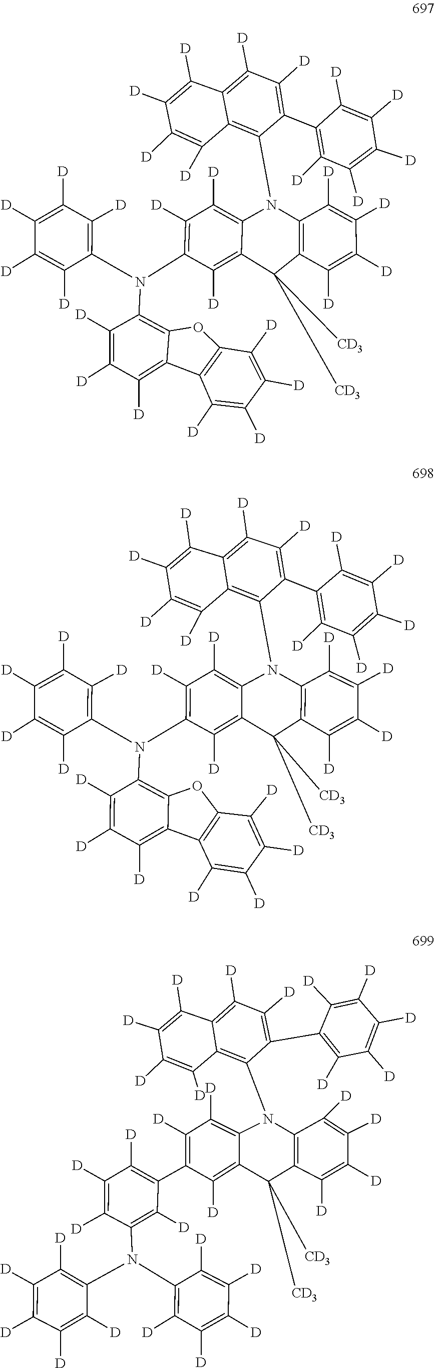

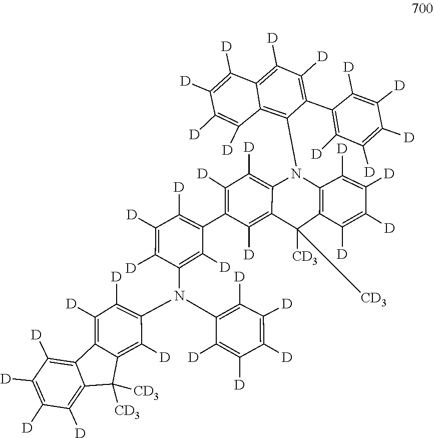 [SMILES: [2H]c1c([2H])c([2H])c(-c2c([2H])c([2H])c3c([2H])c([2H])c([2H])c([2H])c3c2N2c3c([2H])c([2H])c([2H])c([2H])c3C(C([2H])([2H])[2H])(C([2H])([2H])[2H])c3c([2H])c(-c4c([2H])c([2H])c([2H])c(N(c5c([2H])c([2H])c([2H])c([2H])c5[2H])c5c([2H])c([2H])c6c(c5[2H])C(C([2H])([2H])[2H])(C([2H])([2H])[2H])c5c([2H])c([2H])c([2H])c([2H])c5-6)c4[2H])c([2H])c([2H])c32)c([2H])c1[2H]]

Further, compounds having intrinsic properties of the introduced may be synthesized by introducing various substituents into the structure of Chemical Formula 1 above. For example, substances satisfying the conditions required for each organic material layer may be synthesized by introducing substituents mainly used for a hole injection layer material, an electron blocking layer material, a hole transport layer material, a light emitting layer material, an electron transport layer material, a hole blocking layer material, and a charge generation layer material used when manufacturing an organic light emitting device into the core structure.

Further, various substituents are introduced into the structure of Chemical Formula 1 above so that the energy band gap may be enabled to be finely controlled, whereas the properties at the interface between organic materials may be improved, and the use of the substances may be enabled to be diversified.

Further, the present disclosure relates to an organic light emitting device comprising:
a first electrode;
a second electrode provided to face the first electrode; and
one or more organic material layers provided between the first electrode and the second electrode,
wherein one or more layers of the organic material layers comprise the heterocyclic compound represented by Chemical Formula 1 above.

In an embodiment of the present disclosure, the first electrode may be a positive electrode, and the second electrode may be a negative electrode.

In another embodiment, the first electrode may be a negative electrode, and the second electrode may be a positive electrode.

In an embodiment of the present disclosure, the organic light emitting device may be a blue organic light emitting device, and the heterocyclic compound represented by Chemical Formula 1 above may be used as a material of the blue organic light emitting device.

In another embodiment of the present disclosure, the organic light emitting device may be a green organic light emitting device, and the heterocyclic compound represented by Chemical Formula 1 above may be used as a material of the green organic light emitting device.

In another embodiment of the present disclosure, the organic light emitting device may be a red organic light emitting device, and the heterocyclic compound represented by Chemical Formula 1 above may be used as a material of the red organic light emitting device.

In another embodiment of the present disclosure, the organic light emitting device may be a blue organic light emitting device, and the heterocyclic compound represented by Chemical Formula 1 above may be used as a light emitting layer material of the blue organic light emitting device.

In another embodiment of the present disclosure, the organic light emitting device may be a green organic light emitting device, and the heterocyclic compound represented by Chemical Formula 1 above may be used as a light emitting layer material of the green organic light emitting device.

In another embodiment of the present disclosure, the organic light emitting device may be a red organic light emitting device, and the heterocyclic compound represented by Chemical Formula 1 above may be used as a light emitting layer material of the red organic light emitting device.

Specific details of the heterocyclic compound represented by Chemical Formula 1 above are the same as described above.

The organic light emitting device according to the present disclosure may be manufactured by a conventional method and material for manufacturing an organic light emitting device except that an organic material layer with one or more layers is formed using the above-described heterocyclic compound.

The heterocyclic compound may be formed as an organic material layer by a solution application method as well as a vacuum deposition method when manufacturing an organic light emitting device. Here, the solution application method refers to spin coating, dip coating, inkjet printing, screen printing, spraying, roll coating, or the like, but is not limited thereto.

The organic material layer of the organic light emitting device according to the present disclosure may be formed in a single layer structure, but may be formed in a multilayer structure in which an organic material layer with two or more layers is laminated. For example, the organic light emitting device according to the present disclosure may have a structure including a hole injection layer, an electron blocking layer, a hole transport layer, a light emitting layer, an electron transport layer, a hole blocking layer, an electron injection layer, etc. as an organic material layer. However, the structure of the organic light emitting device is not limited thereto and may include an organic material layer with a smaller number of layers.

In an organic light emitting device according to an embodiment of the present disclosure, there is provided an organic light emitting device in which the organic material layer comprising the heterocyclic compound represented by Chemical Formula 1 above further comprises a heterocyclic compound represented by Chemical Formula 3 or Chemical Formula 4 below.

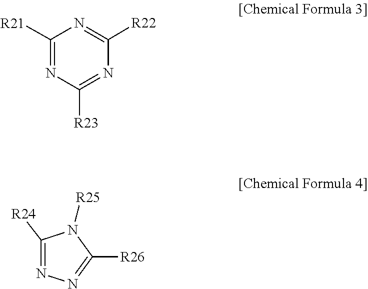

In Chemical Formula 3 and Chemical Formula 4,
R21 to R26 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and a group represented by Chemical Formula 2 above, or two or more groups adjacent to each other are bonded to each other to form a substituted or unsubstituted C6 to C60 aromatic hydrocarbon ring or a substituted or unsubstituted C2 to C60 hetero ring, wherein R101, R102, and R103 are the same as or different from each other, and each independently a substituted or unsubstituted C1 to C60 alkyl group; a substituted or unsubstituted C6 to C60 aryl group; or a substituted or unsubstituted C2 to C60 heteroaryl group.

In an embodiment of the present disclosure, R21 to R26 may be the same as or different from each other, and each independently hydrogen; deuterium; a substituted or unsubstituted C1 to C60 alkyl group; a substituted or unsubstituted C6 to C60 aryl group; or a substituted or unsubstituted C2 to C60 heteroaryl group.

In another embodiment of the present disclosure, R21 to R26 may be the same as or different from each other, and each independently hydrogen; deuterium; a substituted or unsubstituted C1 to C30 alkyl group; a substituted or unsubstituted C6 to C30 aryl group; or a substituted or unsubstituted C2 to C30 heteroaryl group.

In an embodiment of the present disclosure, R21 to R26 may be the same as different from each other, and each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In an embodiment of the present disclosure, R21 to R26 may be the same as or different from each other, and each independently hydrogen; deuterium; a substituted or unsubstituted methyl group; a substituted or unsubstituted ethyl group; a substituted or unsubstituted propyl group; a substituted or unsubstituted isopropyl group; a substituted or unsubstituted butyl group; a substituted or unsubstituted isobutyl group; a substituted or unsubstituted phenyl group; a substituted or unsubstituted biphenyl group; a substituted or unsubstituted naphthyl group; a substituted or unsubstituted phenanthrenyl group; or a substituted or unsubstituted isochrysenyl group.

When the compound represented by Chemical Formula 1 above and the compound represented by any one of Chemical Formula 3 or Chemical Formula 4 above are simultaneously included, better efficiency and lifetime effects are exhibited. From this, when both compounds are included at the same time, it can be expected that an exciplex phenomenon occurs.

The exciplex phenomenon is a phenomenon in which energy having the size of the HOMO energy level of the donor (p-host) and the LUMO energy level of the acceptor (n-host) is emitted through electron exchange between two molecules. When an exciplex phenomenon occurs between two molecules, reverse intersystem crossing (RISC) occurs, and due to this, the internal quantum efficiency of fluorescence may be increased to 100%. When a donor (p-host) with good hole transport ability and an acceptor (n-host) with good electron transport ability are used as hosts of the light emitting layer, since holes are injected into the p-host and electrons are injected into the n-host, the driving voltage can be lowered, thereby helping to improve the lifetime. That is, when the compound represented by Chemical Formula 1 above is used as the donor and the compound represented by Chemical Formula 3 or Chemical Formula 4 above is used as the acceptor, excellent device properties are exhibited.

In an embodiment of the present disclosure, the heterocyclic compound represented by Chemical Formula 3 or Chemical Formula 4 above may be one or more selected from the compounds below.

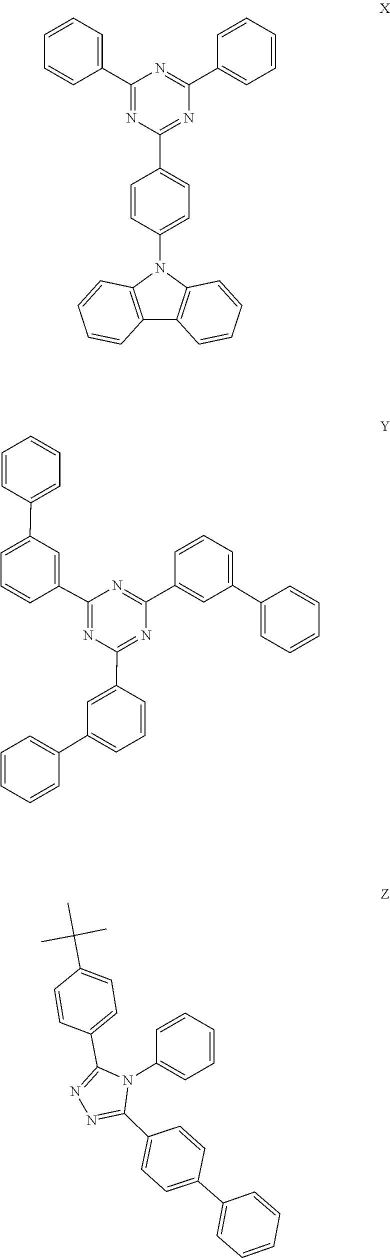 [SMILES: c1ccc(-c2nc(-c3ccccc3)nc(-c3ccc(-n4c5ccccc5c5ccccc54)cc3)n2)cc1 ; c1ccc(-c2cccc(-c3nc(-c4cccc(-c5ccccc5)c4)nc(-c4cccc(-c5ccccc5)c4)n3)c2)cc1 ; CC(C)(C)c1ccc(-c2nnc(-c3ccc(-c4ccccc4)cc3)n2-c2ccccc2)cc1]

Furthermore, an embodiment of the present disclosure provides a composition for an organic material layer of an organic light emitting device comprising the heterocyclic compound represented by Chemical Formula 1 above and the heterocyclic compound represented by Chemical Formula 3 or Chemical Formula 4 above.

Specific details of the heterocyclic compound represented by Chemical Formula 1 above and the heterocyclic compound represented by Chemical Formula 3 or Chemical Formula 4 above are the same as described above.

In an embodiment of the present disclosure, the weight ratio of the heterocyclic compound represented by Chemical Formula 1 above and the heterocyclic compound represented by Chemical Formula 3 or Chemical Formula 4 above in the composition for an organic material layer of the organic light emitting device may be 1:10 to 10:1, 1:8 to 8:1, 1:5 to 5:1, or 1:2 to 2:1, but is not limited thereto.

The composition for an organic material layer of the organic light emitting device may be used when forming the organic material of the organic light emitting device, and in particular, may be more preferably used when forming the host of the hole transport layer, the electron blocking layer, or the light emitting layer.

In an embodiment of the present disclosure, the organic material layer comprises the heterocyclic compound represented by Chemical Formula 1 above and the heterocyclic compound represented by Chemical Formula 3 or Chemical Formula 4 above, and may be used together with a phosphorescent dopant.

As a material for the phosphorescent dopant, those known in the art may be used. For example, phosphorescent dopant materials represented by LL′MX′, LL′L″M, LMX′X″, L2MX′ and L3M may be used, but the scope of the present disclosure is not limited by these examples.

M may be iridium, platinum, osmium, or the like.

L is an anionic bidentate ligand coordinated to M by sp² carbon and a hetero atom, and X may function to trap electrons or holes. Nonlimiting examples of L include 2-(1-naphthyl)benzoxazole, (2-phenylbenzoxazole), (2-(7,8-benzoquinoline), (thiophenylpyridine), phenylpyridine, benzothiophenylpyridine, 3-methoxy-2-phenylpyridine, tolylpyridine, and the like. Nonlimiting examples of X′ and X″ include acetylacetonate (acac), hexafluoroacetylacetonate, salicylidene, picolinate, 8-hydroxyquinolinate, and the like.

Specific examples of the phosphorescent dopant are shown phenylbenzothiazole), below, but are not limited thereto.

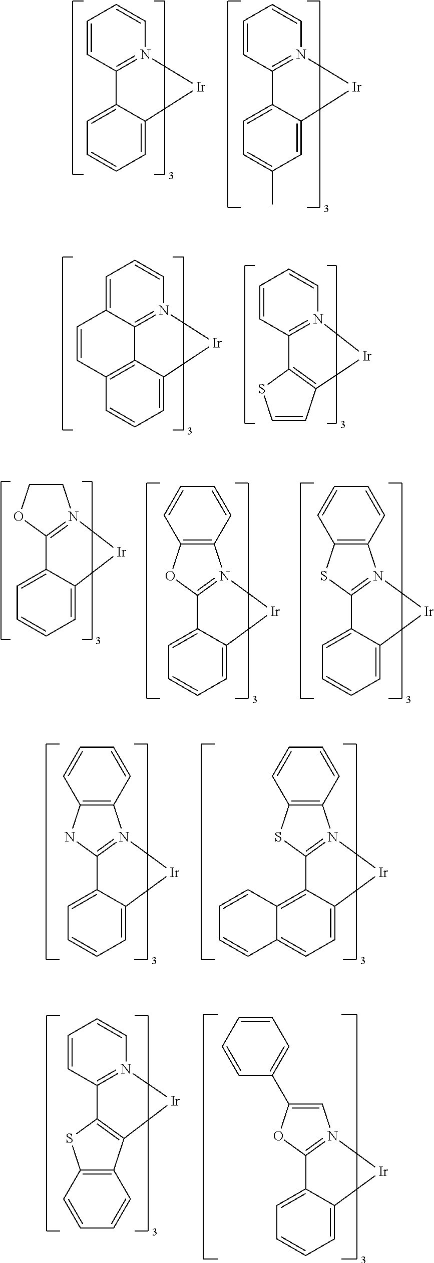

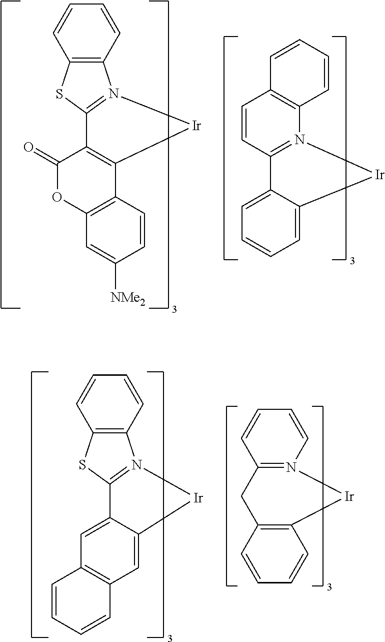

In an embodiment of the present disclosure, the organic material layer may comprise the heterocyclic compound represented by Chemical Formula 1 above and the heterocyclic compound represented by Chemical Formula 3 or 4 above, and may be used together with an iridium-based dopant.

In an embodiment of the present disclosure, Ir(ppy)₃ as a green phosphorescent dopant and (piq)₂(Ir)(acac) as a red phosphorescent dopant may be used as the iridium-based dopant.

In an embodiment of the present disclosure, the dopant may have a content of 1% to 15%, preferably 3% to 10%, and more preferably 3% to 7% based on the total light emitting layer.

In the organic light emitting device according to an embodiment of the present disclosure, the organic material layer may include an electron injection layer or an electron transport layer, and the electron injection layer or the electron transport layer may comprise the heterocyclic compound represented by Chemical Formula 1 above.

In an organic light emitting device according to another embodiment, the organic material layer may include an electron blocking layer or a hole blocking layer, and the electron blocking layer or the hole blocking layer may comprise the heterocyclic compound represented by Chemical Formula 1 above.

In the organic light emitting device according to another embodiment, the organic material layer may include an electron transport layer, a light emitting layer, or a hole blocking layer, and the electron transport layer, the light emitting layer, or the hole blocking layer may comprise the heterocyclic compound represented by Chemical Formula 1 above.

In the organic light emitting device according to another embodiment, the organic material layer may include a light emitting layer, and the light emitting layer may comprise the heterocyclic compound represented by Chemical Formula 1 above.

The organic light emitting device according to an embodiment of the present disclosure may further comprise one or more layers selected from the group consisting of a light emitting layer, a hole injection layer, a hole transport layer, an electron injection layer, an electron transport layer, an electron blocking layer, and a hole blocking layer.

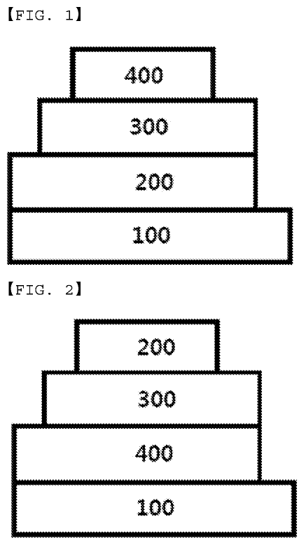

FIGS. 1 to 3 exemplify the lamination order of the electrode and the organic material layer of the organic light emitting device according to an embodiment of the present disclosure. However, it is not intended that the scope of the present application be limited by these drawings, and the structure of an organic light emitting device known in the art may be applied also to the present application.

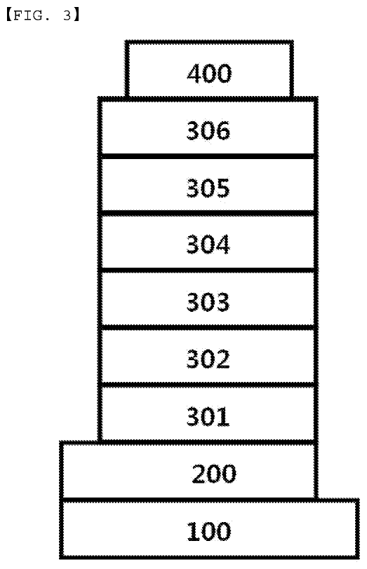

According to FIG. 1, an organic light emitting device in which a positive electrode 200, an organic material layer 300, and a negative electrode 400 are sequentially laminated on a substrate 100 is illustrated. However, it is not limited to such a structure, and as shown in FIG. 2, an organic light emitting device in which a negative electrode, an organic material layer, and a positive electrode are sequentially laminated on a substrate may also be implemented.

FIG. 3 exemplifies a case in which the organic material layer is a multilayer. The organic light emitting device according to FIG. 3 comprises a hole injection layer 301, a hole transport layer 302, a light emitting layer 303, a hole blocking layer 304, an electron transport layer 305, and an electron injection layer 306. However, the scope of the present application is not limited by such a laminated structure, and if necessary, the remaining layers except for the light emitting layer may be omitted, and other necessary functional layers may be further added.

In an embodiment of the present disclosure, there is provided a method for manufacturing an organic light emitting device comprising, the method comprising the steps of:
preparing a substrate;
forming a first electrode on the substrate;
forming one or more organic material layers on the first electrode; and
forming a second electrode on the one or more organic material layers,
wherein the step of forming the one or more organic material layers includes a step of forming the one or more organic material layers using the composition for an organic material layer according to an embodiment of the present disclosure.

In an embodiment of the present disclosure, the step of forming the organic material layer may be pre-mixing the heterocyclic compound represented by Chemical Formula 1 above and the heterocyclic compound represented by Chemical Formula 3 or Chemical Formula 4 above, and forming the organic material layer using a thermal vacuum deposition method.

The pre-mixing refers to mixing the pre-mixed materials in the source after pre-mixing the materials and putting the pre-mixed materials in one source before depositing the heterocyclic compound represented by Chemical Formula 1 above and the heterocyclic compound represented by Chemical Formula 3 or Chemical Formula 4 above on the organic material layer.

The premixed materials may be referred to as a composition for an organic material layer according to an embodiment of the present application.

The organic material layer comprising the heterocyclic compound represented by Chemical Formula 1 above may further comprise other materials as needed.

The organic material layer simultaneously comprising the heterocyclic compound represented by Chemical Formula 1 above and the heterocyclic compound represented by Chemical Formula 3 or Chemical Formula 4 above may further comprise other materials as needed.

In the organic light emitting device according to an embodiment of the present disclosure, materials other than the heterocyclic compound represented by Chemical Formula 1 above or the heterocyclic compound represented by Chemical Formula 3 or Chemical Formula 4 above are exemplified below, but these are for illustrative purposes only, not for limiting the scope of the present application, and may be substituted with materials known in the art.

As a positive electrode material, materials having a relatively high work function may be used, and transparent conductive oxides, metals, conductive polymers, or the like may be used. Specific examples of the positive electrode material may include: metals such as vanadium, chromium, copper, zinc, and gold, or alloys thereof; metal oxides such as zinc oxide, indium oxide, indium tin oxide (ITO), and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole, and polyaniline; etc., but are not limited thereto.

As a negative electrode material, materials having a relatively low work function may be used, and metals, metal oxides, conductive polymers, or the like may be used. Specific examples of the negative electrode material may include: metals such as magnesium, calcium, sodium, potassium, titanium, indium, yttrium, lithium, gadolinium, aluminum, silver, tin, and lead, or alloys thereof; a multilayer structure material such as LiF/Al or LiO²/Al; etc., but are not limited thereto.

As a hole injection layer material, known hole injection layer materials may also be used, and for example, phthalocyanine compounds such as copper phthalocyanine, etc. disclosed in U.S. Pat. No. 4,356,429, or starburst-type amine derivatives written in the literature[Advanced Material, 6, p. 677 (1994)] such as tris(4-carbazoyl-9-ylphenyl)amine (TCTA), 4,4′,4″-tris[phenyl(m-tolyl) amino]triphenylamine (m-MTDATA), 1,3,5-tris[4-(3-methylphenylphenylamino)phenyl]benzene (m-MTDAPB), a soluble conductive polymer of polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), etc. may be used.

As a hole transport layer material, pyrazoline derivatives, arylamine-based derivatives, stilbene derivatives, triphenyldiamine derivatives, etc. may be used, and low molecular weight or high molecular weight materials may also be used.

As an electron transport layer material, metal complexes or the like of oxadiazole derivatives, anthraquinodimethane and its derivatives, benzoquinone and its derivatives, naphthoquinone and its derivatives, anthraquinone and its derivatives, tetracyanoanthraquinodimethane and its derivatives, fluorenone derivatives, diphenyldicyanoethylene and its derivatives, diphenoquinone derivatives, and 8-hydroxyquinoline and its derivatives may be used, and high molecular weight materials as well as low molecular weight materials may also be used.

As an electron injection layer material, for example, LiF is typically used in the art, but the present application is not limited thereto.

As a light emitting layer material, a red, green, or blue light emitting material may be used, and two or more light emitting materials may be mixed and used as needed. At this time, two or more light emitting materials may be deposited and used as individual sources, or may be premixed to be deposited and used as a single source. Further, as a light emitting layer material, a fluorescent material may be used, but a phosphorescent material may also be used. As the light emitting layer material, a material that emits light by combining holes and electrons respectively injected from the positive electrode and the negative electrode may be used alone, but materials in which the host material and the dopant material involve together in light emission may also be used.

When mixing and using the host of the light emitting layer material, hosts of the same series may be mixed and used, or hosts of different series may also be mixed and used. For example, any two or more types of materials of n-type host materials or p-type host materials may be selected and used as a host material of the light emitting layer.

The organic light emitting device according to an embodiment of the present disclosure may be a top emission type, a back emission type, or a double side emission type depending on materials used.

A heterocyclic compound according to an embodiment of the present disclosure may act on a principle similar to that applied to an organic light emitting device even in organic electronic devices including an organic solar cell, an organic photoreceptor, an organic transistor, etc.

### Mode for Carrying Out the Invention

Hereinafter, preferred examples are presented to help the understanding of the present disclosure, but the following examples are only provided for easier understanding of the present disclosure, and the present disclosure is not limited thereto.

### Preparation Examples

### Preparation Example 1. Preparation of Compound 002

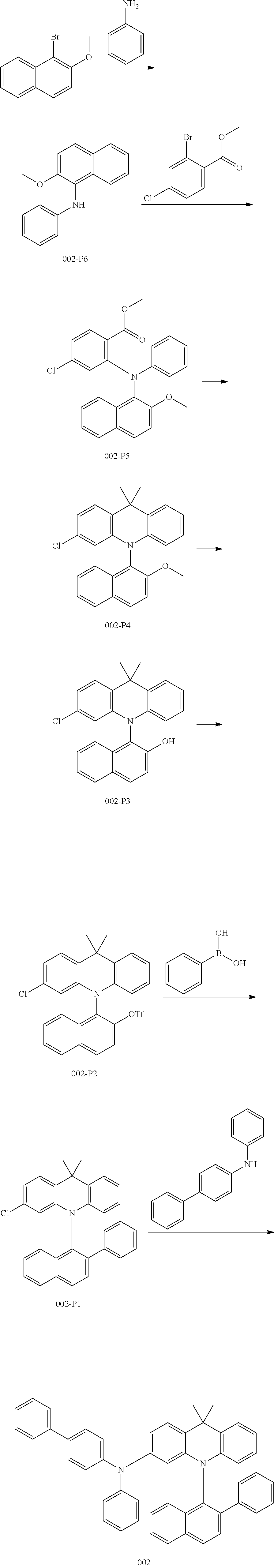

### Preparation Example 1-1. Preparation of Compound 002-P6

50 g (210.89 mmol) of compound 1-bromo-2-methoxynaphthalene and 25.53 g (274.16 mmol) of aniline were dissolved in 500 mL of toluene, 0.95 g (4.22 mmol) of palladium (II) acetate (Pd(OAc)₂), 6.1 g (10.54 mmol) of 4,5-bis(diphenylphosphino)-9,9-dimethylxanthene (xantphos), and 40.53 g (421.78 mmol) of sodium tert-butoxide (t-BuONa) were put therein, and the mixture was stirred under reflux for 2 hours. After completing the reaction, dichloromethane was put in the reaction solution to dissolve it, and then the dissolved solution was extracted with distilled water. Thereafter, the organic layer was dried over anhydrous MgSO₄, the solvent was removed by a rotary evaporator, and the solvent-removed reaction product was purified by column chromatography using dichloromethane and hexane as developing solvents to obtain 40 g (yield 76%) of Compound 002-P6.

### Preparation 1-2. Preparation of Compound 002-P5

40 g (160.44 mmol) of Compound 002-P6 and 52.04 g (208.58 mmol) of methyl 2-bromo-4-chlorobenzoate were dissolved in 500 mL of toluene, 0.72 g (3.21 mmol) of palladium(II) acetate (Pd(OAc)₂), 4.64 g (8.02 mmol) of 4,5-bis(diphenylphosphino)-9,9-dimethylxanthene (xantphos), and 30.84 g (320.89 mmol) of sodium tert-butoxide (t-BuONa) were put therein, and the mixture was stirred under reflux for 2 hours. After completing the reaction, dichloromethane was put in the reaction solution to dissolve it, and then the dissolved solution was extracted with distilled water. Thereafter, the organic layer was dried over anhydrous MgSO₄, the solvent was removed by a rotary evaporator, and the solvent-removed reaction product was purified by column chromatography using dichloromethane and hexane as developing solvents to obtain 45 g (yield 67%) of Compound 002-P5.

### Preparation Example 1-3. Preparation of Compound 002-P4

45 g (107.69 mmol) of Compound 002-P5 was dissolved in 500 mL of tetrahydrofuran, 108 mL (323.06 mmol) of methylmagnesium bromide (3M solution in ether) was slowly added thereto at 0° C., and the mixture was stirred at 60° C. for 6 hours. After finishing the reaction, water was added to the reaction solution to terminate the reaction, and then the reaction product was extracted using dichloromethane and distilled water. Thereafter, the organic layer was dried over anhydrous MgSO₄, and the solvent was removed by a rotary evaporator. Thereafter, after dissolving the solvent-removed reaction product in dichloromethane, boron trifluoride diethyl etherate was added to the reactant, and then the mixture was stirred at room temperature for 4 hours. After finishing the reaction, the reaction product was purified by column chromatography using dichloromethane and hexane as developing solvents to obtain 37 g (yield 86%) of Compound 002-P4.

### Preparation Example 1-4. Preparation of Compound 002-P3

37 g (92.52 mmol) of Compound 002-P4 was dissolved in 400 mL of dichloromethane, 34.77 g (138.78 mmol) of boron tribromide was slowly added thereto at 0° C., and then the mixture was stirred for 3 hours. After finishing the reaction, distilled water was slowly added to the reaction solution to terminate the reaction, the reaction product was extracted with dichloromethane and distilled water, the organic layer was dried over anhydrous MgSO₄, and the solvent was removed by a rotary evaporator. Thereafter, the solvent-removed reaction product was purified by column chromatography using dichloromethane and hexane as developing solvents to obtain 33 g (yield 92%) of Compound 002-P3.

### Preparation Example 1-5. Preparation of Compound 002-P2

33 g (85.52 mmol) of Compound 002-P3 was dissolved in 500 mL of dichloromethane, 10.38 g (102.62 mmol) of triethylamine was added, 28.95 g (102.62 mmol) of trifluoromethanesulfonic (triflic) anhydride was slowly added at 0° C., and the mixture was stirred for 1 hour. After finishing the reaction, distilled water was slowly added to the reaction solution to terminate the reaction, the reaction product was extracted with dichloromethane and distilled water, the organic layer was dried over anhydrous MgSO₄, and the solvent was removed by a rotary evaporator. Thereafter, the solvent-removed reaction product was purified by column chromatography using dichloromethane and hexane as developing solvents to obtain 40 g (yield 90%) of Compound 002-P2.

### Preparation Example 1-6. Preparation of Compound 002-P1

40 g (77.23 mmol) of Compound 002-P1 and 9.89 g (81.09 mmol) of phenylboronic acid were dissolved in 500 mL of toluene, 100 mL of ethanol, and 100 mL of distilled water, 1.78 g (1.54 mmol) of tetrakis(triphenylphosphine) palladium (0) (Pd(PPh₃)₄) and 26.68 g (193.07 mmol) of potassium carbonate (K₂CO₃) were put therein, and the mixture was stirred under reflux for 12 hours. After completing the reaction, the reaction solution was extracted with dichloromethane and distilled water, the organic layer was dried over anhydrous MgSO₄, and the solvent was removed by a rotary evaporator. Thereafter, the solvent-removed reaction product was purified by column chromatography using dichloromethane and hexane as developing solvents to obtain 26 g (yield 75%) of Compound 002-P1.

### Preparation Example 1-7. Preparation of Compound 002

10 g (22.42 mmol) of Compound 002-P1 and 5.50 g (22.42 mmol) of N-phenyl-[1,1′-biphenyl]-4-amine were dissolved in 100 mL of toluene, 0.41 g (0.45 mmol) of tris(dibenzylideneacetone) dipalladium (0) (Pd₂(dba)₃), 0.53 g (1.12 mmol) of dicyclohexyl(2′,4′,6′-triisopropyl-[1,1′-biphenyl]-2-yl)phosphine (Xphos), and 4.31 g (44.85 mmol) of sodium tert-butoxide (t-BuONa) were put therein, and the mixture was stirred under reflux for 2 hours. After completing the reaction, dichloromethane was put in the reaction solution to dissolve it, the dissolved solution was extracted with distilled water, the organic layer was dried over anhydrous MgSO₄, and then the solvent was removed by a rotary evaporator. Thereafter, the solvent-removed reaction product was purified by column chromatography using dichloromethane and hexane as developing solvents to obtain 12 g (yield 82%) of Compound 002.

The target compounds were synthesized as in Table 1 below by preparing them in the same manner as in Preparation Example 1 except that Compound A was used instead of 2-bromo-4-chlorobenzoate, Compound B was used instead of phenylboronic acid, and Compound C was used instead of N-phenyl-[1,1′-biphenyl]-4-amine in Preparation Example 1.

TABLE 1

| Com- | | | | | |
|---|---|---|---|---|---|
| pound | | | | | |
| number | Compound A | Compound B | Compound C | Target compound | Yield |
| 003 | 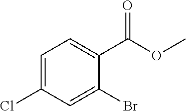 | 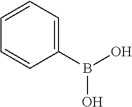 | 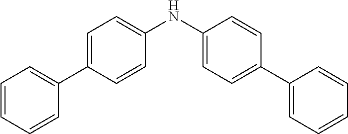 [SMILES: c1ccc(-c2ccc(Nc3ccc(-c4ccccc4)cc3)cc2)cc1] | 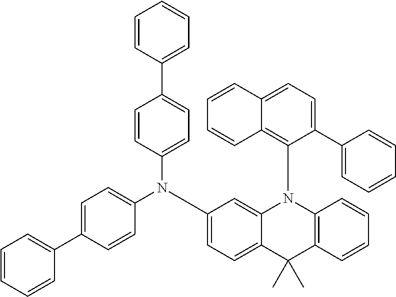 [SMILES: CC1(C)c2ccccc2N(c2c(-c3ccccc3)ccc3ccccc23)c2cc(N(c3ccc(-c4ccccc4)cc3)c3ccc(-c4ccccc4)cc3)ccc21] | 72% |
| 005 | 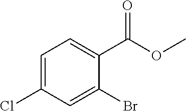 | 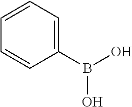 | 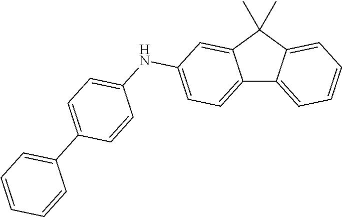 [SMILES: CC1(C)c2ccccc2-c2ccc(Nc3ccc(-c4ccccc4)cc3)cc21] | 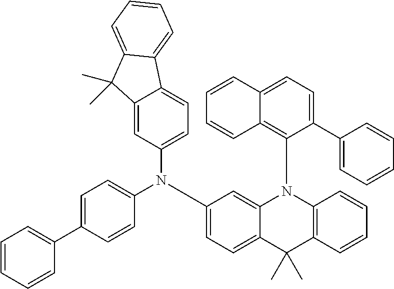 [SMILES: CC1(C)c2ccccc2-c2ccc(N(c3ccc(-c4ccccc4)cc3)c3ccc4c(c3)N(c3c(-c5ccccc5)ccc5ccccc35)c3ccccc3C4(C)C)cc21] | 76% |
| 009 | 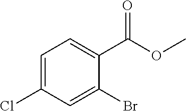 | 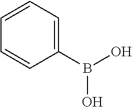 | 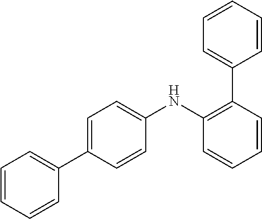 [SMILES: c1ccc(-c2ccc(Nc3ccccc3-c3ccccc3)cc2)cc1] | 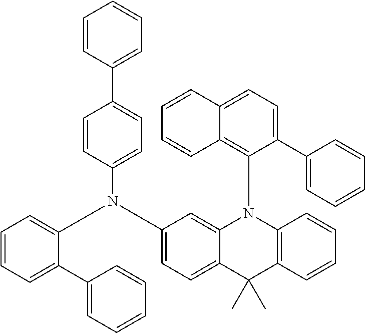 [SMILES: CC1(C)c2ccccc2N(c2c(-c3ccccc3)ccc3ccccc23)c2cc(N(c3ccc(-c4ccccc4)cc3)c3ccccc3-c3ccccc3)ccc21] | 79% |
| 010 | 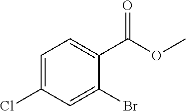 | 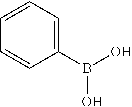 | 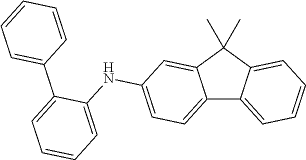 [SMILES: CC1(C)c2ccccc2-c2ccc(Nc3ccccc3-c3ccccc3)cc21] | 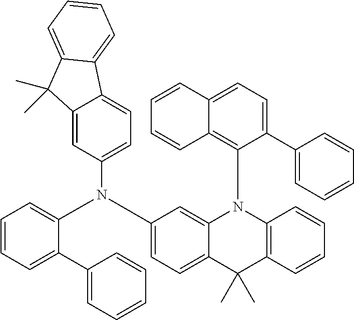 [SMILES: CC1(C)c2ccccc2-c2ccc(N(c3ccc4c(c3)N(c3c(-c5ccccc5)ccc5ccccc35)c3ccccc3C4(C)C)c3ccccc3-c3ccccc3)cc21] | 66% |
| 011 | 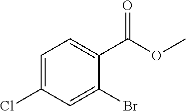 | 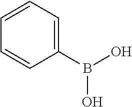 | 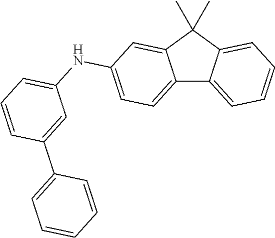 [SMILES: CC1(C)c2ccccc2-c2ccc(Nc3cccc(-c4ccccc4)c3)cc21] | 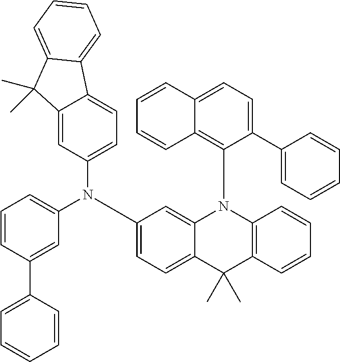 [SMILES: CC1(C)c2ccccc2-c2ccc(N(c3cccc(-c4ccccc4)c3)c3ccc4c(c3)N(c3c(-c5ccccc5)ccc5ccccc35)c3ccccc3C4(C)C)cc21] | 81% |
| 013 | 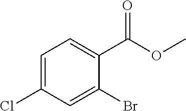 | 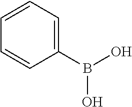 | 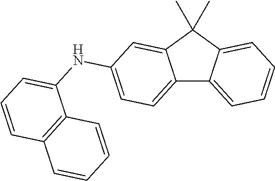 [SMILES: CC1(C)c2ccccc2-c2ccc(Nc3cccc4ccccc34)cc21] | 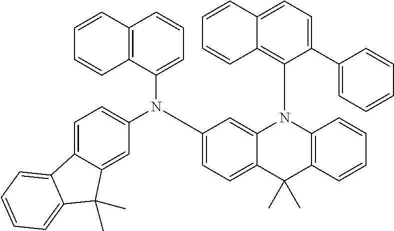 [SMILES: CC1(C)c2ccccc2-c2ccc(N(c3ccc4c(c3)N(c3c(-c5ccccc5)ccc5ccccc35)c3ccccc3C4(C)C)c3cccc4ccccc34)cc21] | 77% |
| 014 | 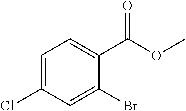 | 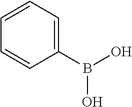 | 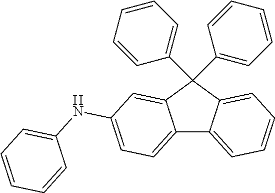 [SMILES: c1ccc(Nc2ccc3c(c2)C(c2ccccc2)(c2ccccc2)c2ccccc2-3)cc1] | 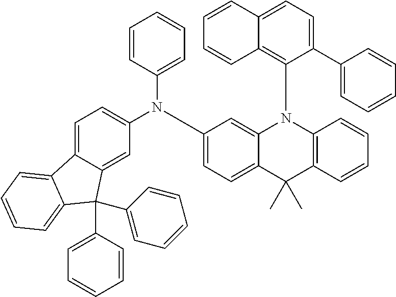 [SMILES: CC1(C)c2ccccc2N(c2c(-c3ccccc3)ccc3ccccc23)c2cc(N(c3ccccc3)c3ccc4c(c3)C(c3ccccc3)(c3ccccc3)c3ccccc3-4)ccc21] | 73% |
| 015 | 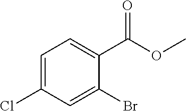 | 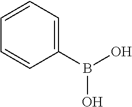 | 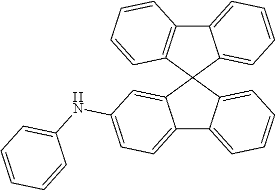 [SMILES: c1ccc(Nc2ccc3c(c2)C2(c4ccccc4-c4ccccc42)c2ccccc2-3)cc1] | 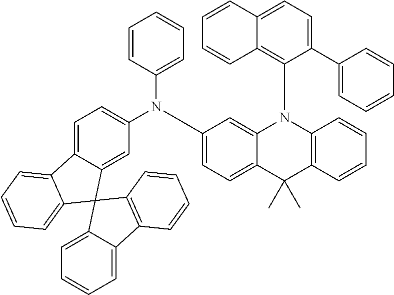 [SMILES: CC1(C)c2ccccc2N(c2c(-c3ccccc3)ccc3ccccc23)c2cc(N(c3ccccc3)c3ccc4c(c3)C3(c5ccccc5-c5ccccc53)c3ccccc3-4)ccc21] | 70% |
| 017 | 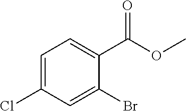 | 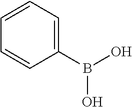 | 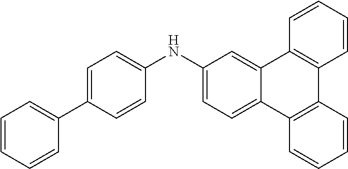 [SMILES: c1ccc(-c2ccc(Nc3ccc4c5ccccc5c5ccccc5c4c3)cc2)cc1] | 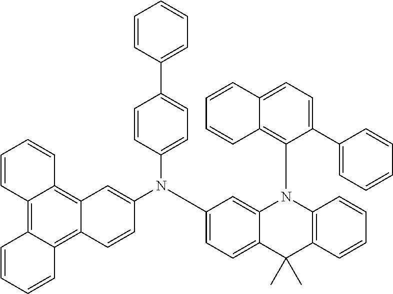 [SMILES: CC1(C)c2ccccc2N(c2c(-c3ccccc3)ccc3ccccc23)c2cc(N(c3ccc(-c4ccccc4)cc3)c3ccc4c5ccccc5c5ccccc5c4c3)ccc21] | 72% |
| 019 | 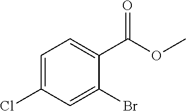 | 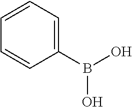 | 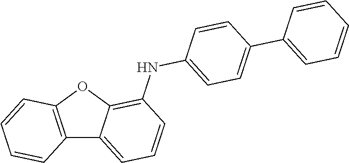 [SMILES: c1ccc(-c2ccc(Nc3cccc4c3oc3ccccc34)cc2)cc1] | 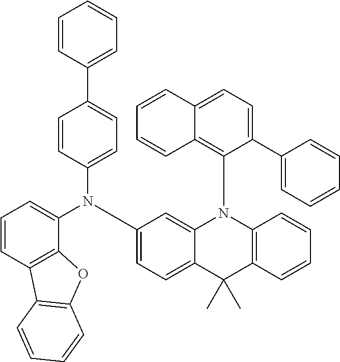 [SMILES: CC1(C)c2ccccc2N(c2c(-c3ccccc3)ccc3ccccc23)c2cc(N(c3ccc(-c4ccccc4)cc3)c3cccc4c3oc3ccccc34)ccc21] | 68% |
| 024 | 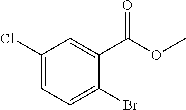 | 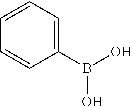 | 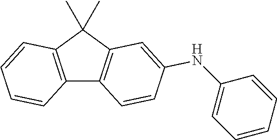 [SMILES: CC1(C)c2ccccc2-c2ccc(Nc3ccccc3)cc21] | 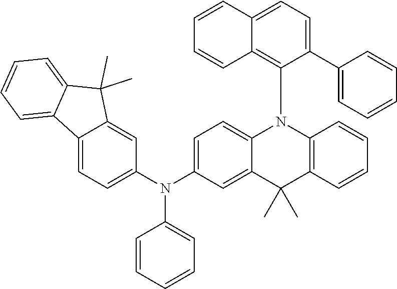 [SMILES: CC1(C)c2ccccc2-c2ccc(N(c3ccccc3)c3ccc4c(c3)C(C)(C)c3ccccc3N4c3c(-c4ccccc4)ccc4ccccc34)cc21] | 68% |
| 038 | 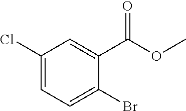 | 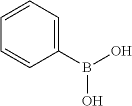 | 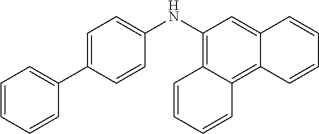 [SMILES: c1ccc(-c2ccc(Nc3cc4ccccc4c4ccccc34)cc2)cc1] | 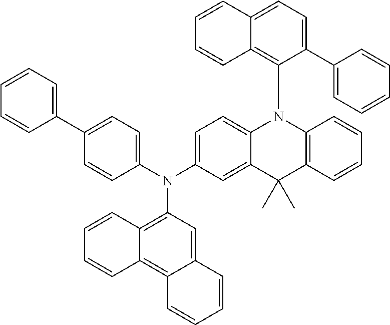 [SMILES: CC1(C)c2ccccc2N(c2c(-c3ccccc3)ccc3ccccc23)c2ccc(N(c3ccc(-c4ccccc4)cc3)c3cc4ccccc4c4ccccc34)cc21] | 79% |
| 040 | 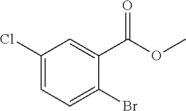 | 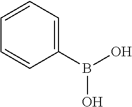 | 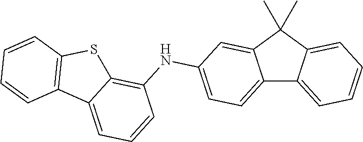 [SMILES: CC1(C)c2ccccc2-c2ccc(Nc3cccc4c3sc3ccccc34)cc21] | 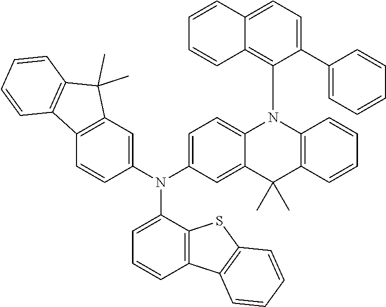 [SMILES: CC1(C)c2ccccc2-c2ccc(N(c3ccc4c(c3)C(C)(C)c3ccccc3N4c3c(-c4ccccc4)ccc4ccccc34)c3cccc4c3sc3ccccc34)cc21] | 76% |
| 046 | 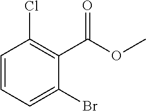 | 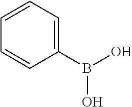 | 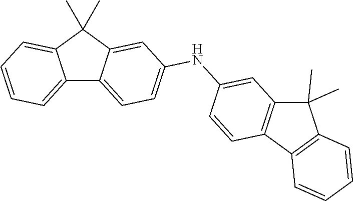 [SMILES: CC1(C)c2ccccc2-c2ccc(Nc3ccc4c(c3)C(C)(C)c3ccccc3-4)cc21] | 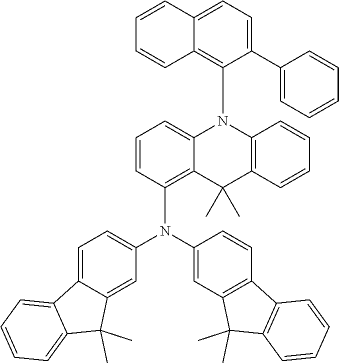 [SMILES: CC1(C)c2ccccc2-c2ccc(N(c3ccc4c(c3)C(C)(C)c3ccccc3-4)c3cccc4c3C(C)(C)c3ccccc3N4c3c(-c4ccccc4)ccc4ccccc34)cc21] | 67% |
| 053 | 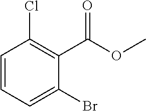 | 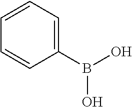 | 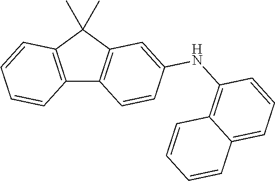 [SMILES: CC1(C)c2ccccc2-c2ccc(Nc3cccc4ccccc34)cc21] | 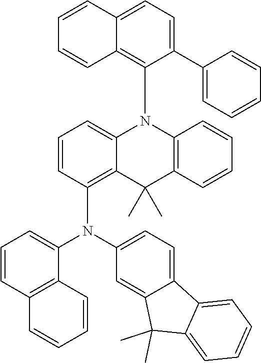 [SMILES: CC1(C)c2ccccc2-c2ccc(N(c3cccc4c3C(C)(C)c3ccccc3N4c3c(-c4ccccc4)ccc4ccccc34)c3cccc4ccccc34)cc21] | 80% |
| 064 | 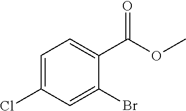 | 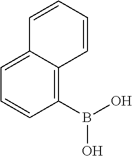 [SMILES: OB(O)c1cccc2ccccc12] | 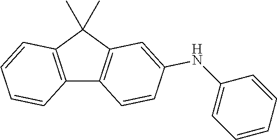 [SMILES: CC1(C)c2ccccc2-c2ccc(Nc3ccccc3)cc21] | 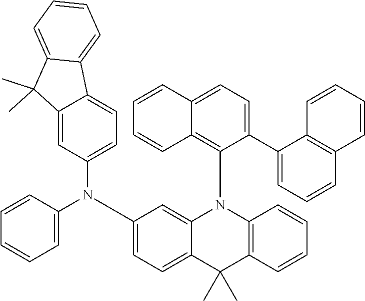 [SMILES: CC1(C)c2ccccc2-c2ccc(N(c3ccccc3)c3ccc4c(c3)N(c3c(-c5cccc6ccccc56)ccc5ccccc35)c3ccccc3C4(C)C)cc21] | 79% |
| 072 | 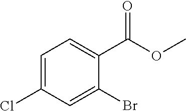 | 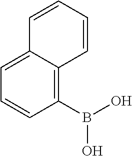 [SMILES: OB(O)c1cccc2ccccc12] | 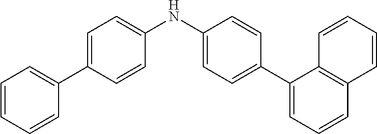 [SMILES: c1ccc(-c2ccc(Nc3ccc(-c4cccc5ccccc45)cc3)cc2)cc1] | 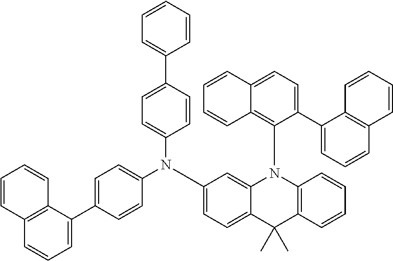 [SMILES: CC1(C)c2ccccc2N(c2c(-c3cccc4ccccc34)ccc3ccccc23)c2cc(N(c3ccc(-c4ccccc4)cc3)c3ccc(-c4cccc5ccccc45)cc3)ccc21] | 71% |
| 093 | 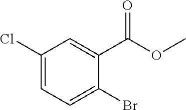 | 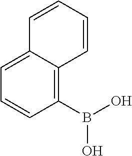 [SMILES: OB(O)c1cccc2ccccc12] | 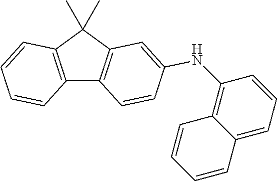 [SMILES: CC1(C)c2ccccc2-c2ccc(Nc3cccc4ccccc34)cc21] | 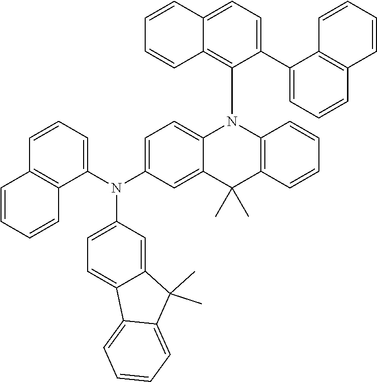 [SMILES: CC1(C)c2ccccc2-c2ccc(N(c3ccc4c(c3)C(C)(C)c3ccccc3N4c3c(-c4cccc5ccccc45)ccc4ccccc34)c3cccc4ccccc34)cc21] | 71% |
| 098 | 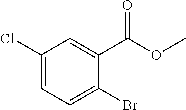 | 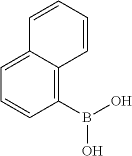 [SMILES: OB(O)c1cccc2ccccc12] | 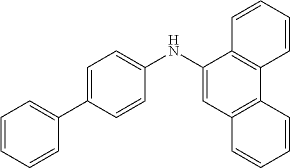 [SMILES: c1ccc(-c2ccc(Nc3cc4ccccc4c4ccccc34)cc2)cc1] | 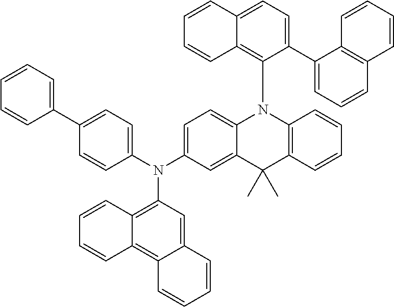 [SMILES: CC1(C)c2ccccc2N(c2c(-c3cccc4ccccc34)ccc3ccccc23)c2ccc(N(c3ccc(-c4ccccc4)cc3)c3cc4ccccc4c4ccccc34)cc21] | 74% |
| 105 | 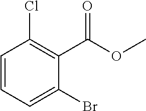 | 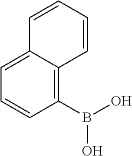 [SMILES: OB(O)c1cccc2ccccc12] | 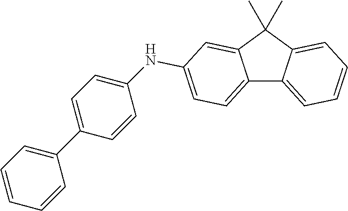 [SMILES: CC1(C)c2ccccc2-c2ccc(Nc3ccc(-c4ccccc4)cc3)cc21] | 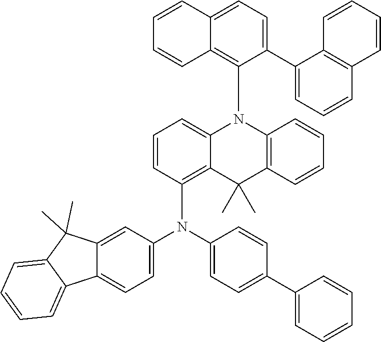 [SMILES: CC1(C)c2ccccc2-c2ccc(N(c3ccc(-c4ccccc4)cc3)c3cccc4c3C(C)(C)c3ccccc3N4c3c(-c4cccc5ccccc45)ccc4ccccc34)cc21] | 78% |
| 109 | 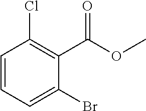 | 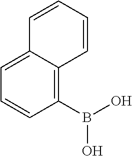 [SMILES: OB(O)c1cccc2ccccc12] | 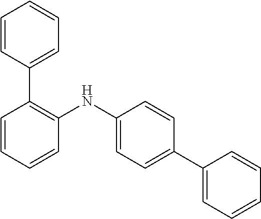 [SMILES: c1ccc(-c2ccc(Nc3ccccc3-c3ccccc3)cc2)cc1] | 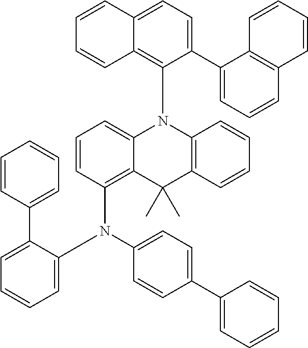 [SMILES: CC1(C)c2ccccc2N(c2c(-c3cccc4ccccc34)ccc3ccccc23)c2cccc(N(c3ccc(-c4ccccc4)cc3)c3ccccc3-c3ccccc3)c21] | 73% |
| 116 | 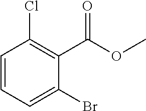 | 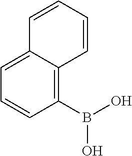 [SMILES: OB(O)c1cccc2ccccc12] | 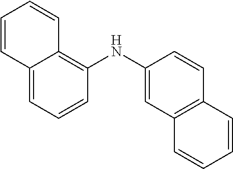 [SMILES: c1ccc2cc(Nc3cccc4ccccc34)ccc2c1] | 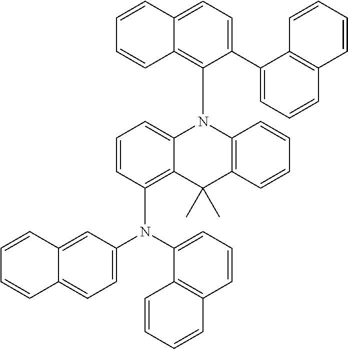 [SMILES: CC1(C)c2ccccc2N(c2c(-c3cccc4ccccc34)ccc3ccccc23)c2cccc(N(c3ccc4ccccc4c3)c3cccc4ccccc34)c21] | 76% |
| 127 | 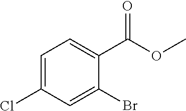 | 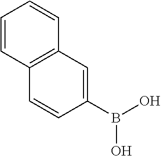 [SMILES: OB(O)c1ccc2ccccc2c1] | 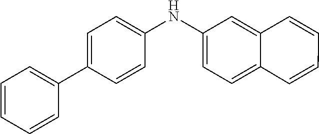 [SMILES: c1ccc(-c2ccc(Nc3ccc4ccccc4c3)cc2)cc1] | 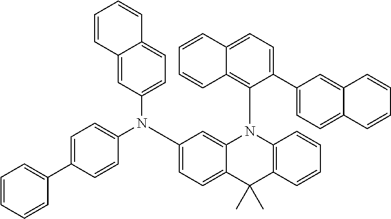 [SMILES: CC1(C)c2ccccc2N(c2c(-c3ccc4ccccc4c3)ccc3ccccc23)c2cc(N(c3ccc(-c4ccccc4)cc3)c3ccc4ccccc4c3)ccc21] | 75% |
| 131 | 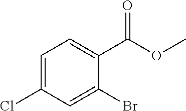 | 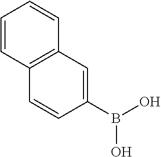 [SMILES: OB(O)c1ccc2ccccc2c1] | 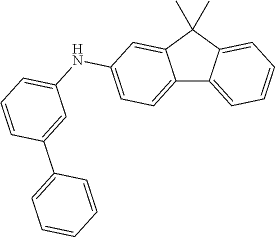 [SMILES: CC1(C)c2ccccc2-c2ccc(Nc3cccc(-c4ccccc4)c3)cc21] | 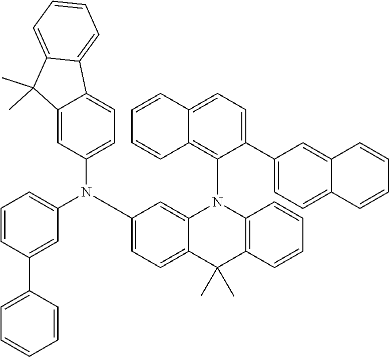 [SMILES: CC1(C)c2ccccc2-c2ccc(N(c3cccc(-c4ccccc4)c3)c3ccc4c(c3)N(c3c(-c5ccc6ccccc6c5)ccc5ccccc35)c3ccccc3C4(C)C)cc21] | 67% |
| 143 | 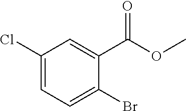 | 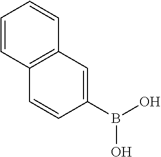 [SMILES: OB(O)c1ccc2ccccc2c1] | 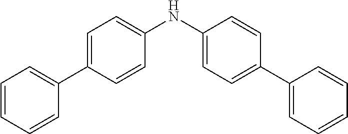 [SMILES: c1ccc(-c2ccc(Nc3ccc(-c4ccccc4)cc3)cc2)cc1] | 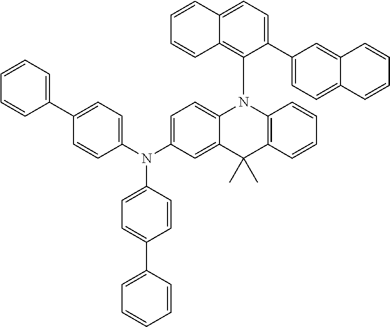 [SMILES: CC1(C)c2ccccc2N(c2c(-c3ccc4ccccc4c3)ccc3ccccc23)c2ccc(N(c3ccc(-c4ccccc4)cc3)c3ccc(-c4ccccc4)cc3)cc21] | 71% |
| 159 | 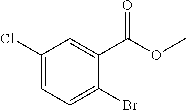 | 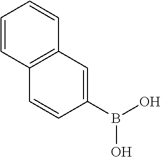 [SMILES: OB(O)c1ccc2ccccc2c1] | 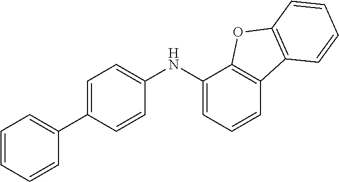 [SMILES: c1ccc(-c2ccc(Nc3cccc4c3oc3ccccc34)cc2)cc1] | 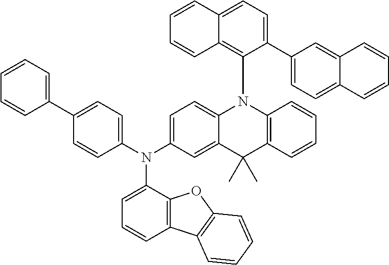 [SMILES: CC1(C)c2ccccc2N(c2c(-c3ccc4ccccc4c3)ccc3ccccc23)c2ccc(N(c3ccc(-c4ccccc4)cc3)c3cccc4c3oc3ccccc34)cc21] | 77% |
| 164 | 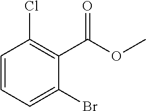 | 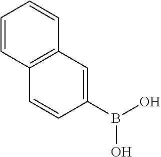 [SMILES: OB(O)c1ccc2ccccc2c1] | 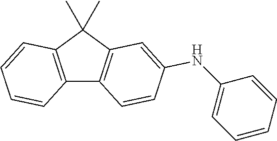 [SMILES: CC1(C)c2ccccc2-c2ccc(Nc3ccccc3)cc21] | 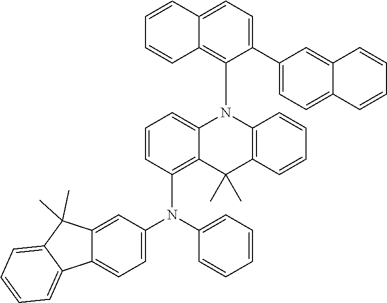 [SMILES: CC1(C)c2ccccc2-c2ccc(N(c3ccccc3)c3cccc4c3C(C)(C)c3ccccc3N4c3c(-c4ccc5ccccc5c4)ccc4ccccc34)cc21] | 76% |
| 182 | 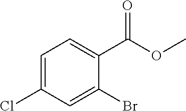 | 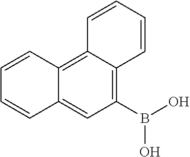 [SMILES: OB(O)c1cc2ccccc2c2ccccc12] | 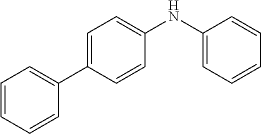 [SMILES: c1ccc(Nc2ccc(-c3ccccc3)cc2)cc1] | 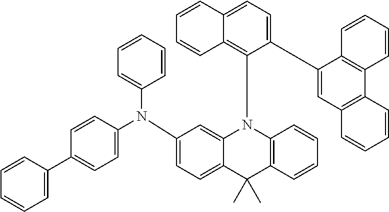 [SMILES: CC1(C)c2ccccc2N(c2c(-c3cc4ccccc4c4ccccc34)ccc3ccccc23)c2cc(N(c3ccccc3)c3ccc(-c4ccccc4)cc3)ccc21] | 71% |
| 202 | 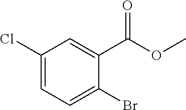 | 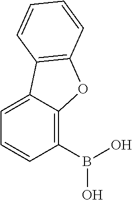 [SMILES: OB(O)c1cccc2c1oc1ccccc12] | 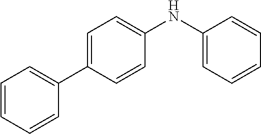 [SMILES: c1ccc(Nc2ccc(-c3ccccc3)cc2)cc1] | 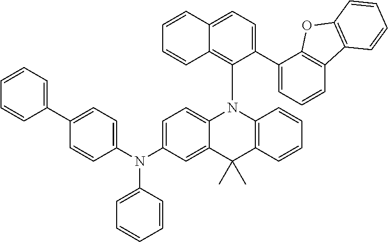 [SMILES: CC1(C)c2ccccc2N(c2c(-c3cccc4c3oc3ccccc34)ccc3ccccc23)c2ccc(N(c3ccccc3)c3ccc(-c4ccccc4)cc3)cc21] | 74% |

### Preparation Example 2. Preparation of Compound 322

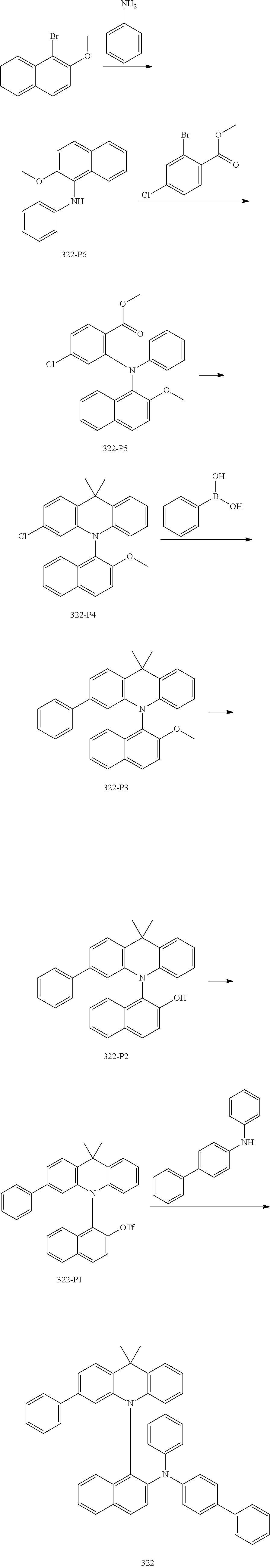

### Preparation Example 2-1. Preparation of Compound 322-P6

50 g (210.89 mmol) of compound 1-bromo-2-methoxynaphthalene and 25.53 g (274.16 mmol) of aniline were dissolved in 500 mL of toluene, 0.95 g (4.22 mmol) of palladium(II) acetate (Pd(OAc)₂), 6.1 g (10.54 mmol) of 4,5-bis(diphenylphosphino)-9,9-dimethylxanthene (xantphos), and 40.53 g (421.78 mmol) of sodium tert-butoxide (t-BuONa) were put therein, and the mixture was stirred under reflux for 2 hours. After completing the reaction, dichloromethane was put in the reaction solution to dissolve it, and then the dissolved solution was extracted with distilled water. Thereafter, the organic layer was dried over anhydrous MgSO₄, the solvent was removed by a rotary evaporator, and the solvent-removed reaction product was purified by column chromatography using dichloromethane and hexane as developing solvents to obtain 40 g (yield 76%) of Compound 322-P6.

### Preparation Example 2-2. Preparation of Compound 322-P5

40 g (160.44 mmol) of Compound 322-P6 and 52.04 g (208.58 mmol) of methyl 2-bromo-4-chlorobenzoate were dissolved in 500 mL of toluene, 0.72 g (3.21 mmol) of palladium(II) acetate (Pd(OAc)₂), 4.64 g (8.02 mmol) of 4,5-bis(diphenylphosphino)-9,9-dimethylxanthene (xantphos), and 30.84 g (320.89 mmol) of sodium tert-butoxide (t-BuONa) were put therein, and the mixture was stirred under reflux for 2 hours. After completing the reaction, dichloromethane was put in the reaction solution to dissolve it, and then the dissolved solution was extracted with distilled water. Thereafter, the organic layer was dried over anhydrous MgSO₄, the solvent was removed by a rotary evaporator, and the solvent-removed reaction product was purified by column chromatography using dichloromethane and hexane as developing solvents to obtain 45 g (yield 67%) of Compound 322-P5.

### Preparation Example 2-3. Preparation of Compound 322-P4

45 g (107.69 mmol) of Compound 322-P5 was dissolved in 500 mL of tetrahydrofuran, 108 mL (323.06 mmol) of methylmagnesium bromide (3M solution in ether) was slowly added thereto at 0° C., and then the mixture was stirred at 60° C. for 6 hours. After completing the reaction, water was added to the reaction solution to terminate the reaction, and then the reaction product was extracted using dichloromethane and distilled water. Thereafter, the organic layer was dried over anhydrous MgSO₄, and then the solvent was removed by a rotary evaporator. Thereafter, after dissolving the solvent-removed reaction product in dichloromethane, boron trifluoride diethyl etherate was added to the reactant, and then the mixture was stirred at room temperature for 4 hours. After finishing the reaction, the reaction product was purified by column chromatography using dichloromethane and hexane as developing solvents to obtain 37 g (yield 86%) of Compound 322-P4.

### Preparation Example 2-4. Preparation of Compound 322-P3

37 g (92.52 mmol) of Compound 322-P4 and 12.41 g (101.77 mmol) of phenylboronic acid were dissolved in 500 mL of 1,4-dioxane and 100 mL of distilled water, 1.06 g (1.85 mmol) of bis(dibenzylideneacetone) palladium (0) (Pd(dba)₂), 2.21 g (4.63 mmol) of dicyclohexyl(2′,4′,6′-triisopropyl-[1,1′-biphenyl]-2-yl)phosphine (Xphos), and 31.97 g (231.30 mmol) of potassium carbonate (K₂CO₃) were put therein, and the mixture was stirred under reflux for 12 hours. After completing the reaction, the reaction solution was extracted with dichloromethane and distilled water, the organic layer was dried over anhydrous MgSO₄, the solvent was removed by a rotary evaporator, and then the solvent-removed reaction product was purified by column chromatography using dichloromethane and hexane as developing solvents to obtain 30 g (yield 73%) of Compound 322-P3.

### Preparation Example 2-5. Preparation of Compound 322-P2

37 g (83.79 mmol) of Compound 322-P3 was dissolved in 400 mL of dichloromethane, 31.49 g (125.69 mmol) of boron tribromide was slowly added thereto at 0° C., and then the mixture was stirred for 3 hours. After finishing the reaction, distilled water was slowly added to the reaction solution to terminate the reaction, the reaction product was extracted with dichloromethane and distilled water, the organic layer was dried over anhydrous MgSO₄, and then the solvent was removed by a rotary evaporator. Thereafter, the solvent-removed reaction product was purified by column chromatography using dichloromethane and hexane as developing solvents to obtain 32 g (yield 89%) of Compound 322-P2.

### Preparation Example 2-6. Preparation of Compound 322-P1

32 g (74.85 mmol) of Compound 322-P2 was dissolved in 500 mL of dichloromethane, 9.09 g (89.82 mmol) of triethylamine was added, 25.34 (89.82 g mmol) of trifluoromethanesulfonic (triflic) anhydride was slowly added at 0° C., and then the mixture was stirred for 1 hour. After finishing the reaction, distilled water was slowly added to the reaction solution to terminate the reaction, the reaction product was extracted with dichloromethane and distilled water, the organic layer was dried over anhydrous MgSO₄, and then the solvent was removed by a rotary evaporator. Thereafter, the solvent-removed reaction product was purified by column chromatography using dichloromethane and hexane as developing solvents to obtain 38 g (yield 91%) of Compound 322-P1.

### Preparation Example 2-7. Preparation of Compound 322

10 g (17.87 mmol) of Compound 322-P1 and 4.60 g (18.76 mmol) of N-phenyl-[1,1′-biphenyl]-4-amine were dissolved in 100 mL of toluene, 0.33 (0.36 mmol) of g tris(dibenzylideneacetone) dipalladium (0) (Pd₂(dba)₃), 0.52 g (0.89 mmol) of 4,5-bis(diphenylphosphino)-9,9-dimethylxanthene (xantphos), and 3.43 g (35.74 mmol) of sodium tert-butoxide (t-BuONa) were put therein, and the mixture was stirred under reflux for 2 hours. After completing the reaction, dichloromethane was put in the reaction solution to dissolve it, the dissolved solution was extracted with distilled water, the organic layer was dried over anhydrous MgSO₄, and then the solvent was removed by a rotary evaporator. Thereafter, the solvent-removed reaction product was purified by column chromatography using dichloromethane and hexane as developing solvents to obtain 9 g (yield 77%) of Compound 322.

The target compounds were synthesized as in Table 2 below by preparing them in the same manner as in Preparation Example 2 except that Compound D was used instead of 2-bromo-4-chlorobenzoate, Compound E was used instead of phenylboronic acid, and Compound F was used instead of N-phenyl-[1,1′-biphenyl]-4-amine in Preparation Example 2.

TABLE 2

| Com- | | | | | |
|---|---|---|---|---|---|
| pound | | | | | |
| number | Compound D | Compound E | Compound F | Target compound | Yield |
| 325 | 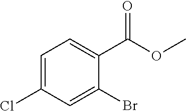 | 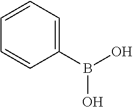 | 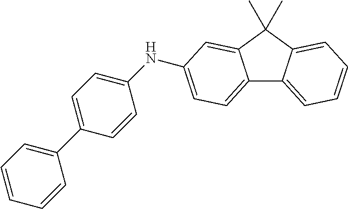 [SMILES: CC1(C)c2ccccc2-c2ccc(Nc3ccc(-c4ccccc4)cc3)cc21] | 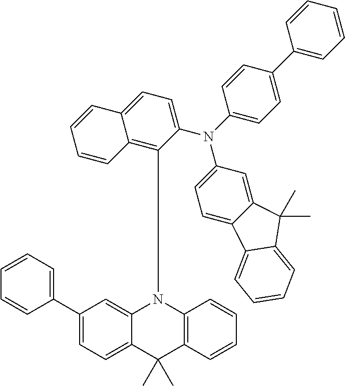 [SMILES: CC1(C)c2ccccc2-c2ccc(N(c3ccc(-c4ccccc4)cc3)c3ccc4ccccc4c3CCCN3c4ccccc4C(C)(C)c4ccc(-c5ccccc5)cc43)cc21] | 75% |
| 343 | 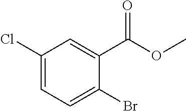 | 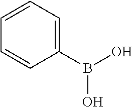 | 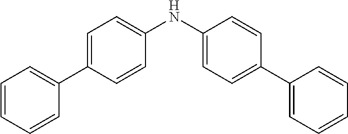 [SMILES: c1ccc(-c2ccc(Nc3ccc(-c4ccccc4)cc3)cc2)cc1] | 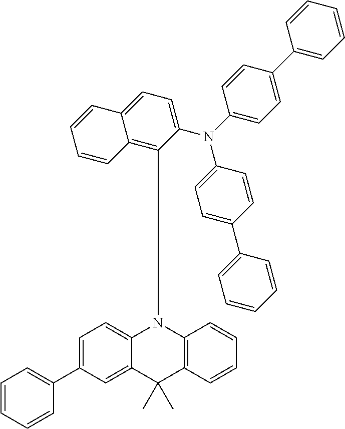 [SMILES: CC1(C)c2ccccc2N(CCCc2c(N(c3ccc(-c4ccccc4)cc3)c3ccc(-c4ccccc4)cc3)ccc3ccccc23)c2ccc(-c3ccccc3)cc21] | 71% |
| 352 | 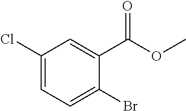 | 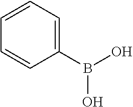 | 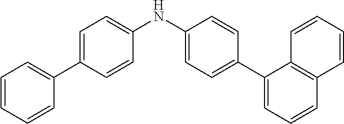 [SMILES: c1ccc(-c2ccc(Nc3ccc(-c4cccc5ccccc45)cc3)cc2)cc1] | 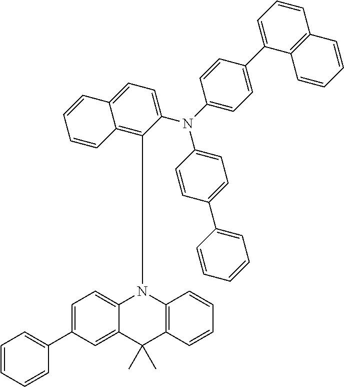 [SMILES: CC1(C)c2ccccc2N(CCCc2c(N(c3ccc(-c4ccccc4)cc3)c3ccc(-c4cccc5ccccc45)cc3)ccc3ccccc23)c2ccc(-c3ccccc3)cc21] | 69% |
| 367 | 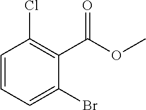 | 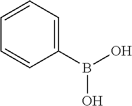 | 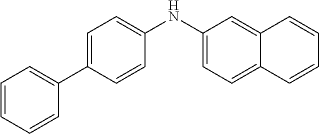 [SMILES: c1ccc(-c2ccc(Nc3ccc4ccccc4c3)cc2)cc1] | 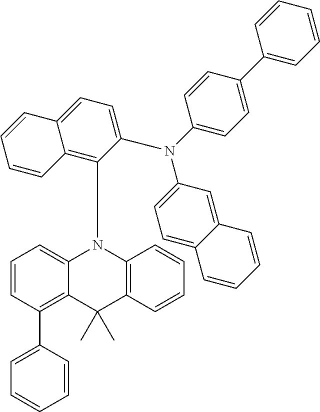 [SMILES: CC1(C)c2ccccc2N(c2c(N(c3ccc(-c4ccccc4)cc3)c3ccc4ccccc4c3)ccc3ccccc23)c2cccc(-c3ccccc3)c21] | 80% |
| 384 | 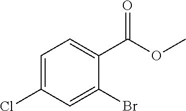 | 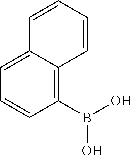 [SMILES: OB(O)c1cccc2ccccc12] | 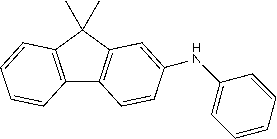 [SMILES: CC1(C)c2ccccc2-c2ccc(Nc3ccccc3)cc21] | 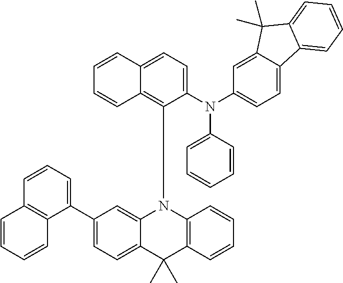 [SMILES: CC1(C)c2ccccc2-c2ccc(N(c3ccccc3)c3ccc4ccccc4c3N3c4ccccc4C(C)(C)c4ccc(-c5cccc6ccccc56)cc43)cc21] | 78% |
| 393 | 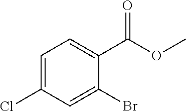 | 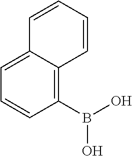 [SMILES: OB(O)c1cccc2ccccc12] | 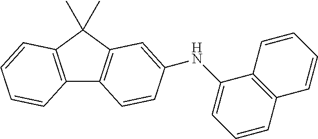 [SMILES: CC1(C)c2ccccc2-c2ccc(Nc3cccc4ccccc34)cc21] | 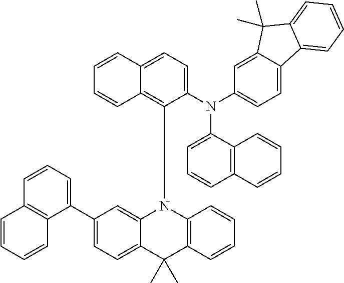 [SMILES: CC1(C)c2ccccc2-c2ccc(N(c3ccc4ccccc4c3N3c4ccccc4C(C)(C)c4ccc(-c5cccc6ccccc56)cc43)c3cccc4ccccc34)cc21] | 78% |
| 397 | 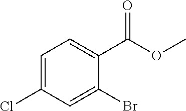 | 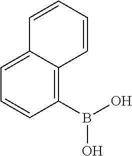 [SMILES: OB(O)c1cccc2ccccc12] | 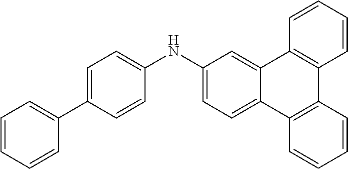 [SMILES: c1ccc(-c2ccc(Nc3ccc4c5ccccc5c5ccccc5c4c3)cc2)cc1] | 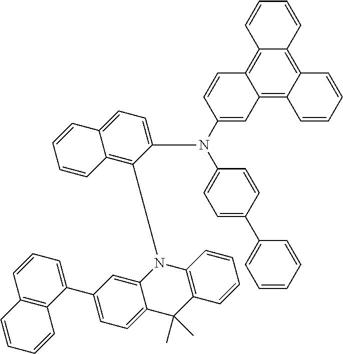 [SMILES: CC1(C)c2ccccc2N(c2c(N(c3ccc(-c4ccccc4)cc3)c3ccc4c5ccccc5c5ccccc5c4c3)ccc3ccccc23)c2cc(-c3cccc4ccccc34)ccc21] | 74% |
| 402 | 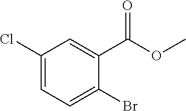 | 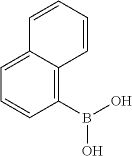 [SMILES: OB(O)c1cccc2ccccc12] | 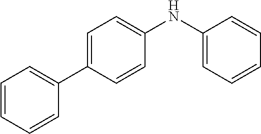 [SMILES: c1ccc(Nc2ccc(-c3ccccc3)cc2)cc1] | 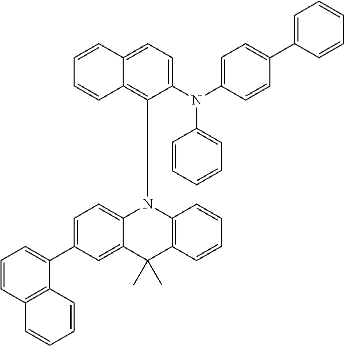 [SMILES: CC1(C)c2ccccc2N(c2c(N(c3ccccc3)c3ccc(-c4ccccc4)cc3)ccc3ccccc23)c2ccc(-c3cccc4ccccc34)cc21] | 72% |
| 419 | 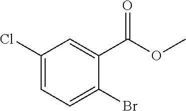 | 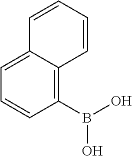 [SMILES: OB(O)c1cccc2ccccc12] | 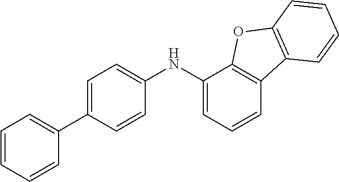 [SMILES: c1ccc(-c2ccc(Nc3cccc4c3oc3ccccc34)cc2)cc1] | 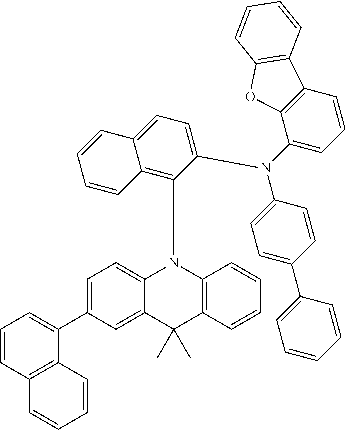 [SMILES: CC1(C)c2ccccc2N(c2c(N(c3ccc(-c4ccccc4)cc3)c3cccc4c3oc3ccccc34)ccc3ccccc23)c2ccc(-c3cccc4ccccc34)cc21] | 68% |
| 425 | 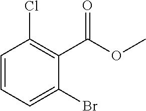 | 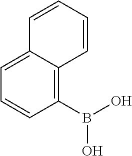 [SMILES: OB(O)c1cccc2ccccc12] | 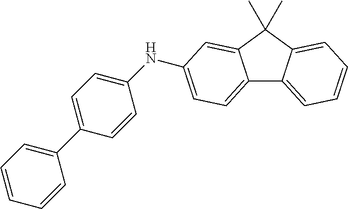 [SMILES: CC1(C)c2ccccc2-c2ccc(Nc3ccc(-c4ccccc4)cc3)cc21] | 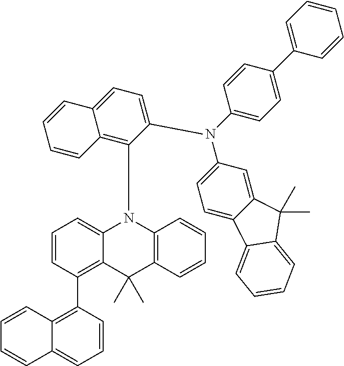 [SMILES: CC1(C)c2ccccc2-c2ccc(N(c3ccc(-c4ccccc4)cc3)c3ccc4ccccc4c3N3c4ccccc4C(C)(C)c4c(-c5cccc6ccccc56)cccc43)cc21] | 70% |
| 431 | 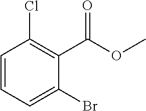 | 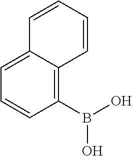 [SMILES: OB(O)c1cccc2ccccc12] | 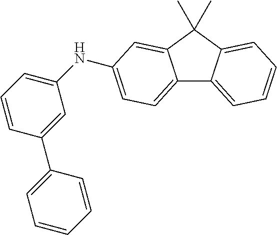 [SMILES: CC1(C)c2ccccc2-c2ccc(Nc3cccc(-c4ccccc4)c3)cc21] | 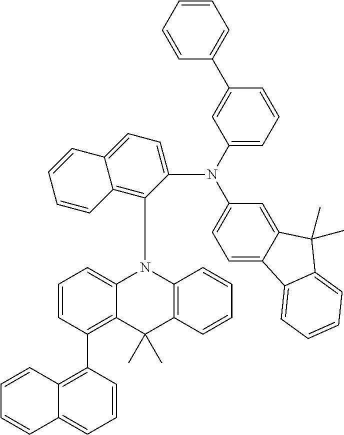 [SMILES: CC1(C)c2ccccc2-c2ccc(N(c3cccc(-c4ccccc4)c3)c3ccc4ccccc4c3N3c4ccccc4C(C)(C)c4c(-c5cccc6ccccc56)cccc43)cc21] | 73% |
| 444 | 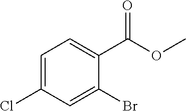 | 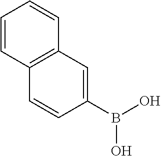 [SMILES: OB(O)c1ccc2ccccc2c1] | 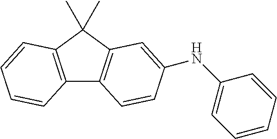 [SMILES: CC1(C)c2ccccc2-c2ccc(Nc3ccccc3)cc21] | 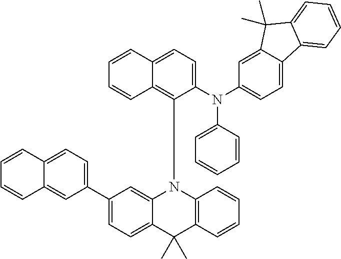 [SMILES: CC1(C)c2ccccc2-c2ccc(N(c3ccccc3)c3ccc4ccccc4c3N3c4ccccc4C(C)(C)c4ccc(-c5ccc6ccccc6c5)cc43)cc21] | 77% |
| 458 | 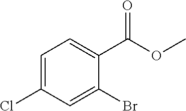 | 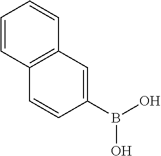 [SMILES: OB(O)c1ccc2ccccc2c1] | 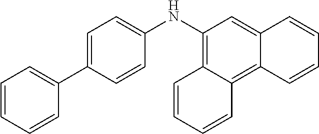 [SMILES: c1ccc(-c2ccc(Nc3cc4ccccc4c4ccccc34)cc2)cc1] | 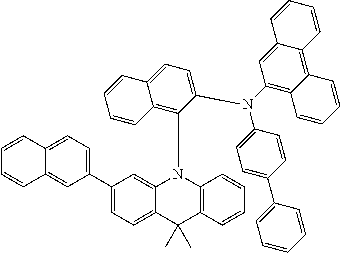 [SMILES: CC1(C)c2ccccc2N(c2c(N(c3ccc(-c4ccccc4)cc3)c3cc4ccccc4c4ccccc34)ccc3ccccc23)c2cc(-c3ccc4ccccc4c3)ccc21] | 76% |
| 467 | 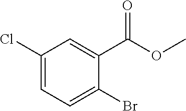 | 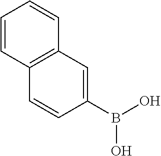 [SMILES: OB(O)c1ccc2ccccc2c1] | 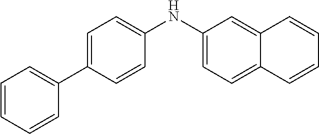 [SMILES: c1ccc(-c2ccc(Nc3ccc4ccccc4c3)cc2)cc1] | 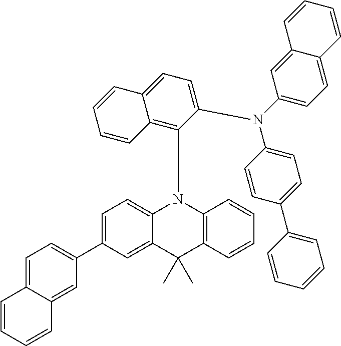 [SMILES: CC1(C)c2ccccc2N(c2c(N(c3ccc(-c4ccccc4)cc3)c3ccc4ccccc4c3)ccc3ccccc23)c2ccc(-c3ccc4ccccc4c3)cc21] | 75% |
| 481 | 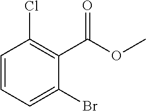 | 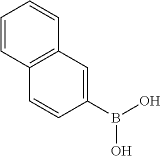 [SMILES: OB(O)c1ccc2ccccc2c1] | 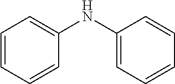 [SMILES: c1ccc(Nc2ccccc2)cc1] | 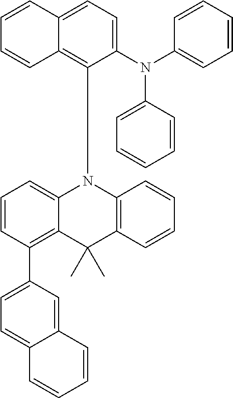 [SMILES: CC1(C)c2ccccc2N(c2c(N(c3ccccc3)c3ccccc3)ccc3ccccc23)c2cccc(-c3ccc4ccccc4c3)c21] | 78% |
| 493 | 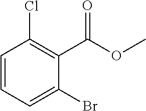 | 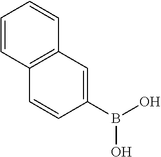 [SMILES: OB(O)c1ccc2ccccc2c1] | 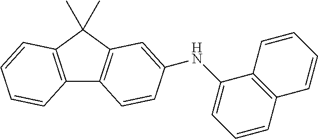 [SMILES: CC1(C)c2ccccc2-c2ccc(Nc3cccc4ccccc34)cc21] | 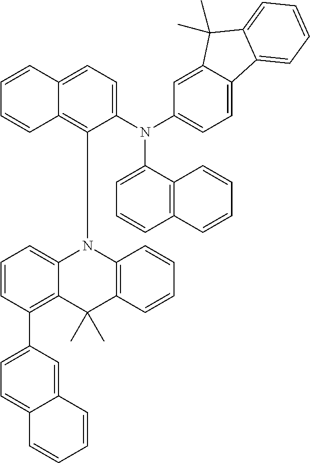 [SMILES: CC1(C)c2ccccc2-c2ccc(N(c3ccc4ccccc4c3N3c4ccccc4C(C)(C)c4c(-c5ccc6ccccc6c5)cccc43)c3cccc4ccccc34)cc21] | 72% |
| 506 | 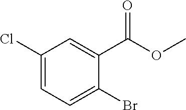 | 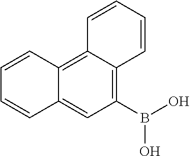 [SMILES: OB(O)c1cc2ccccc2c2ccccc12] | 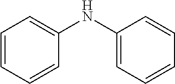 [SMILES: c1ccc(Nc2ccccc2)cc1] | 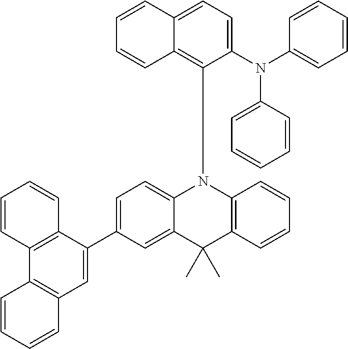 [SMILES: CC1(C)c2ccccc2N(c2c(N(c3ccccc3)c3ccccc3)ccc3ccccc23)c2ccc(-c3cc4ccccc4c4ccccc34)cc21] | 70% |
| 515 | 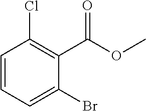 | 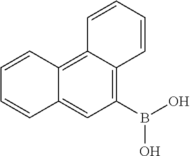 [SMILES: OB(O)c1cc2ccccc2c2ccccc12] | 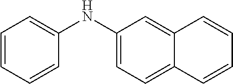 [SMILES: c1ccc(Nc2ccc3ccccc3c2)cc1] | 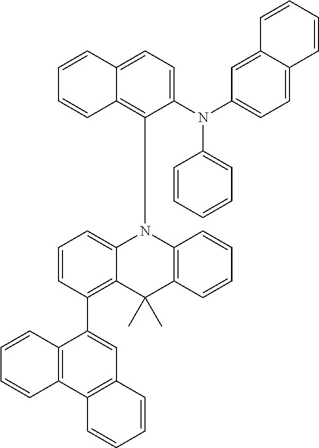 [SMILES: CC1(C)c2ccccc2N(c2c(N(c3ccccc3)c3ccc4ccccc4c3)ccc3ccccc23)c2cccc(-c3cc4ccccc4c4ccccc34)c21] | 73% |
| 531 | 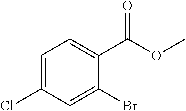 | 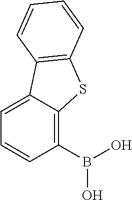 [SMILES: OB(O)c1cccc2c1sc1ccccc12] | 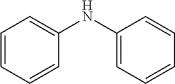 [SMILES: c1ccc(Nc2ccccc2)cc1] | 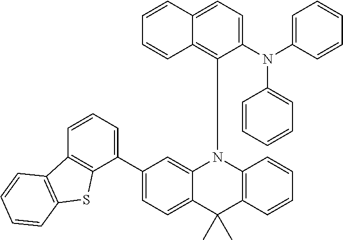 [SMILES: CC1(C)c2ccccc2N(c2c(N(c3ccccc3)c3ccccc3)ccc3ccccc23)c2cc(-c3cccc4c3sc3ccccc34)ccc21] | 78% |
| 540 | 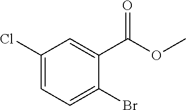 | 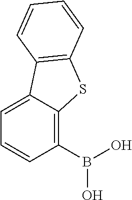 [SMILES: OB(O)c1cccc2c1sc1ccccc12] | 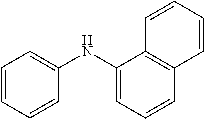 [SMILES: c1ccc(Nc2cccc3ccccc23)cc1] | 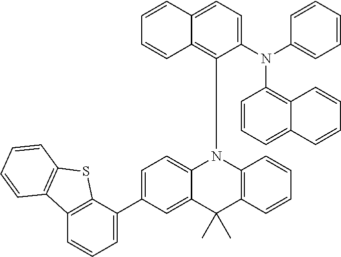 [SMILES: CC1(C)c2ccccc2N(c2c(N(c3ccccc3)c3cccc4ccccc34)ccc3ccccc23)c2ccc(-c3cccc4c3sc3ccccc34)cc21] | 68% |
| 546 | 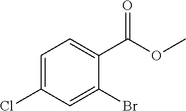 | 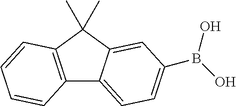 [SMILES: CC1(C)c2ccccc2-c2ccc(B(O)O)cc21] | 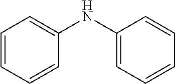 [SMILES: c1ccc(Nc2ccccc2)cc1] | 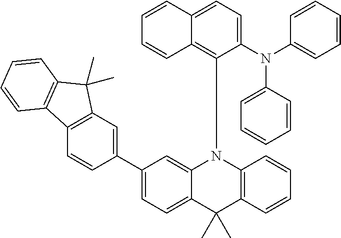 [SMILES: CC1(C)c2ccccc2-c2ccc(-c3ccc4c(c3)N(c3c(N(c5ccccc5)c5ccccc5)ccc5ccccc35)c3ccccc3C4(C)C)cc21] | 77% |
| 553 | 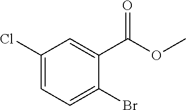 | 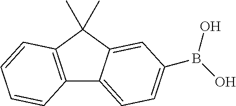 [SMILES: CC1(C)c2ccccc2-c2ccc(B(O)O)cc21] | 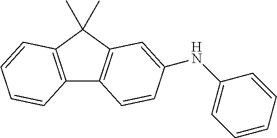 [SMILES: CC1(C)c2ccccc2-c2ccc(Nc3ccccc3)cc21] | 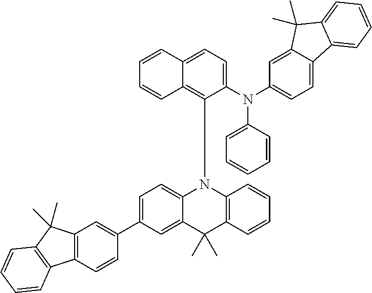 [SMILES: CC1(C)c2ccccc2-c2ccc(-c3ccc4c(c3)C(C)(C)c3ccccc3N4c3c(N(c4ccccc4)c4ccc5c(c4)C(C)(C)c4ccccc4-5)ccc4ccccc34)cc21] | 75% |

### Preparation Example 3. Preparation of Compound 199

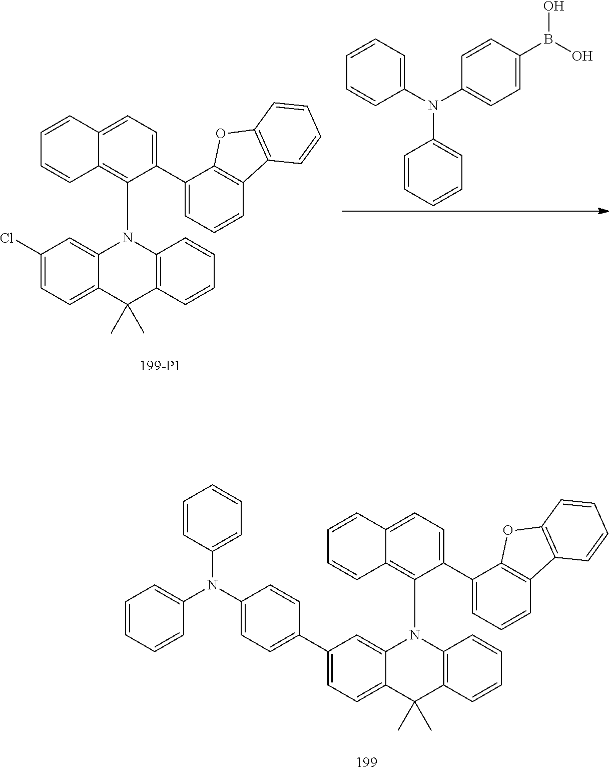

10 g (18.65 mmol) of Compound 199-P1 and 5.66 g (19.59 mmol) of 4-(diphenylamino)phenylboronic acid were dissolved in 100 mL of 1,4-dioxane and 20 mL of distilled water, 0.21 g (0.37 mol) of bis(dibenzylideneacetone) palladium(0) (Pd(dba)₂), 0.44 g (0.93 mmol) of dicyclohexyl(2′,4′,6′-triisopropyl-[1,1′-biphenyl]-2-yl)phosphine (Xphos), and 6.45 g (46.64 mmol) of potassium carbonate (K₂CO₃) were put therein, and the mixture was stirred under reflux for 12 hours. After completing the reaction, the reaction solution was extracted with dichloromethane and distilled water, the organic layer was dried over anhydrous MgSO₄, and then the solvent was removed by a rotary evaporator. Thereafter, the solvent-removed reaction product was purified by column chromatography using dichloromethane and hexane as developing solvents to obtain 10 g (yield 72%) of Compound 199.

The target compounds were synthesized as in Table 3 below by preparing them in the same manner as in Preparation Example 3 except that Compound G was used instead of Compound 199-P1, and Compound H was used instead of 4-(diphenylamino)phenylboronic acid in Preparation Example 3.

TABLE 3

| Com- | | | | |
|---|---|---|---|---|
| pound | | | | |
| number | Compound G | Compound H | Target compound | Yield |
| 224 | 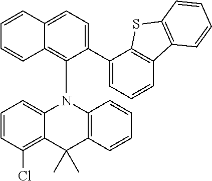 [SMILES: CC1(C)c2ccccc2N(c2c(-c3cccc4c3sc3ccccc34)ccc3ccccc23)c2cccc(Cl)c21] | 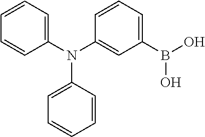 [SMILES: OB(O)c1cccc(N(c2ccccc2)c2ccccc2)c1] | 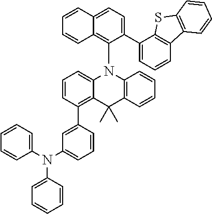 [SMILES: CC1(C)c2ccccc2N(c2c(-c3cccc4c3sc3ccccc34)ccc3ccccc23)c2cccc(-c3cccc(N(c4ccccc4)c4ccccc4)c3)c21] | 70% |
| 243 | 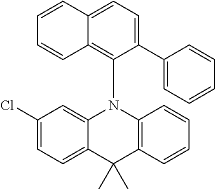 [SMILES: CC1(C)c2ccccc2N(c2c(-c3ccccc3)ccc3ccccc23)c2cc(Cl)ccc21] | 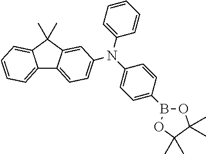 [SMILES: CC1(C)c2ccccc2-c2ccc(N(c3ccccc3)c3ccc(B4OC(C)(C)C(C)(C)O4)cc3)cc21] | 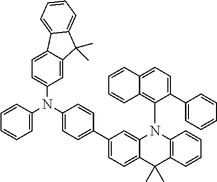 [SMILES: CC1(C)c2ccccc2-c2ccc(N(c3ccccc3)c3ccc(-c4ccc5c(c4)N(c4c(-c6ccccc6)ccc6ccccc46)c4ccccc4C5(C)C)cc3)cc21] | 73% |
| 249 | 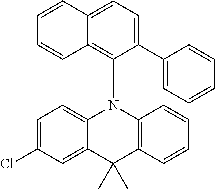 [SMILES: CC1(C)c2ccccc2N(c2c(-c3ccccc3)ccc3ccccc23)c2ccc(Cl)cc21] | 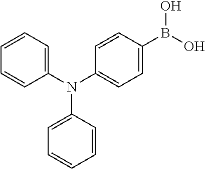 [SMILES: OB(O)c1ccc(N(c2ccccc2)c2ccccc2)cc1] | 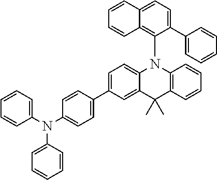 [SMILES: CC1(C)c2ccccc2N(c2c(-c3ccccc3)ccc3ccccc23)c2ccc(-c3ccc(N(c4ccccc4)c4ccccc4)cc3)cc21] | 68% |
| 273 | 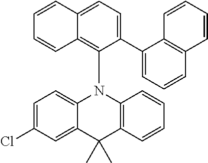 [SMILES: CC1(C)c2ccccc2N(c2c(-c3cccc4ccccc34)ccc3ccccc23)c2ccc(Cl)cc21] | 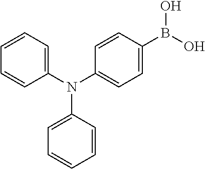 [SMILES: OB(O)c1ccc(N(c2ccccc2)c2ccccc2)cc1] | 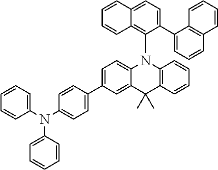 [SMILES: CC1(C)c2ccccc2N(c2c(-c3cccc4ccccc34)ccc3ccccc23)c2ccc(-c3ccc(N(c4ccccc4)c4ccccc4)cc3)cc21] | 76% |
| 287 | 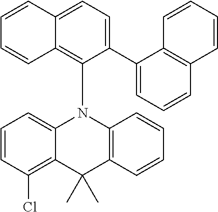 [SMILES: CC1(C)c2ccccc2N(c2c(-c3cccc4ccccc34)ccc3ccccc23)c2cccc(Cl)c21] | 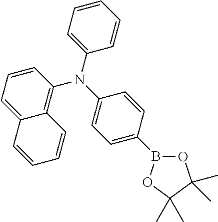 [SMILES: CC1(C)OB(c2ccc(N(c3ccccc3)c3cccc4ccccc34)cc2)OC1(C)C] | 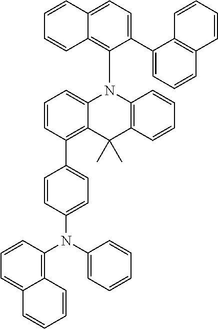 [SMILES: CC1(C)c2ccccc2N(c2c(-c3cccc4ccccc34)ccc3ccccc23)c2cccc(-c3ccc(N(c4ccccc4)c4cccc5ccccc45)cc3)c21] | 72% |
| 298 | 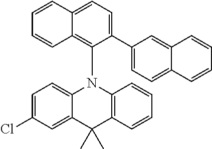 [SMILES: CC1(C)c2ccccc2N(c2c(-c3ccc4ccccc4c3)ccc3ccccc23)c2ccc(Cl)cc21] | 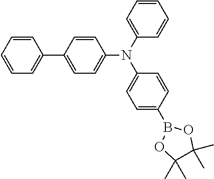 [SMILES: CC1(C)OB(c2ccc(N(c3ccccc3)c3ccc(-c4ccccc4)cc3)cc2)OC1(C)C] | 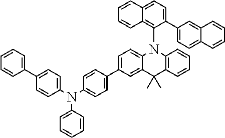 [SMILES: CC1(C)c2ccccc2N(c2c(-c3ccc4ccccc4c3)ccc3ccccc23)c2ccc(-c3ccc(N(c4ccccc4)c4ccc(-c5ccccc5)cc4)cc3)cc21] | 78% |
| 305 | 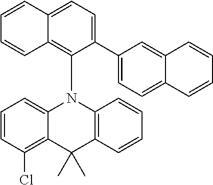 [SMILES: CC1(C)c2ccccc2N(c2c(-c3ccc4ccccc4c3)ccc3ccccc23)c2cccc(Cl)c21] | 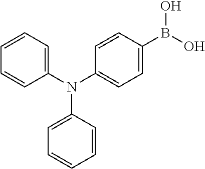 [SMILES: OB(O)c1ccc(N(c2ccccc2)c2ccccc2)cc1] | 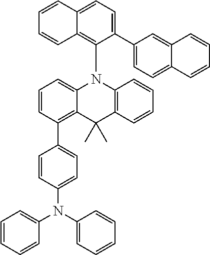 [SMILES: CC1(C)c2ccccc2N(c2c(-c3ccc4ccccc4c3)ccc3ccccc23)c2cccc(-c3ccc(N(c4ccccc4)c4ccccc4)cc3)c21] | 71% |
| 312 | 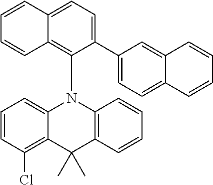 [SMILES: CC1(C)c2ccccc2N(c2c(-c3ccc4ccccc4c3)ccc3ccccc23)c2cccc(Cl)c21] | 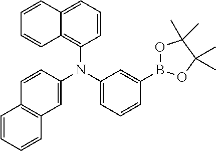 [SMILES: CC1(C)OB(c2cccc(N(c3ccc4ccccc4c3)c3cccc4ccccc34)c2)OC1(C)C] | 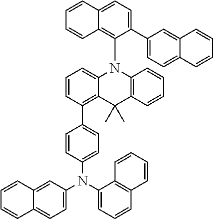 [SMILES: CC1(C)c2ccccc2N(c2c(-c3ccc4ccccc4c3)ccc3ccccc23)c2cccc(-c3ccc(N(c4ccc5ccccc5c4)c4cccc5ccccc45)cc3)c21] | 77% |
| 319 | 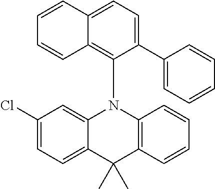 [SMILES: CC1(C)c2ccccc2N(c2c(-c3ccccc3)ccc3ccccc23)c2cc(Cl)ccc21] | 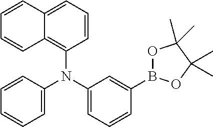 [SMILES: CC1(C)OB(c2cccc(N(c3ccccc3)c3cccc4ccccc34)c2)OC1(C)C] | 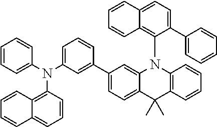 [SMILES: CC1(C)c2ccccc2N(c2c(-c3ccccc3)ccc3ccccc23)c2cc(-c3cccc(N(c4ccccc4)c4cccc5ccccc45)c3)ccc21] | 75% |

### Preparation Example 4. Preparation of Compound 562

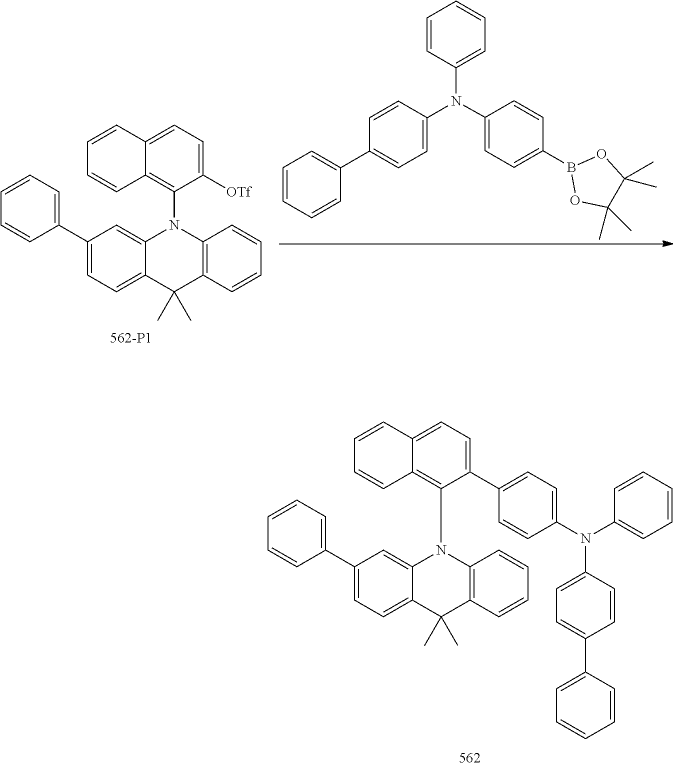

10 g (17.87 mmol) of Compound 562-P1 and 8.39 g (18.76 mmol) of N-phenyl-N-(4-(4,4,5,5-tetramethyl-1,3,2-dioxaborolan-2-yl)phenyl)-[1,1′-biphenyl]-4-amine were dissolved in 100 mL of toluene, 20 mL of ethanol, and 20 mL of distilled water, 0.41 g (0.36 mmol) of tetrakis(triphenylphosphine) palladium (0) (Pd(PPh₃)₄) and 6.17 g (44.67 mmol) of potassium carbonate (K₂CO₃) were put therein, and the mixture was stirred under reflux for 12 hours. After completing the reaction, the reaction solution was extracted with dichloromethane and distilled water, the organic layer was dried over anhydrous MgSO₄, and then the solvent was removed by a rotary evaporator. Thereafter, the solvent-removed reaction product was purified by column chromatography using dichloromethane and hexane as developing solvents to obtain 10 g (yield 77%) of Compound 562.

The target compounds were synthesized as in Table 4 below by preparing them in the same manner as in Preparation Example 4 except that Compound I was used instead of Compound 562-P1, and Compound J was used instead of N-phenyl-N-(4-(4,4,5,5-tetramethyl-1,3,2-dioxaborolan-2-yl)phenyl)-[1,1′-biphenyl]-4-amine in Preparation Example 4.

TABLE 4

| Com- | | | | |
|---|---|---|---|---|
| pound | | | | |
| number | Compound I | Compound J | Target compound | Yield |
| 580 | 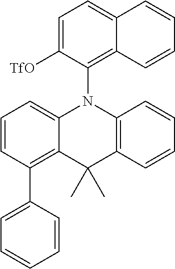 [SMILES: CC1(C)c2ccccc2N(c2c(OS(=O)(=O)C(F)(F)F)ccc3ccccc23)c2cccc(-c3ccccc3)c21] | 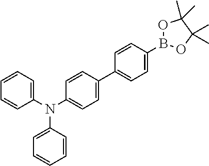 [SMILES: CC1(C)OB(c2ccc(-c3ccc(N(c4ccccc4)c4ccccc4)cc3)cc2)OC1(C)C] | 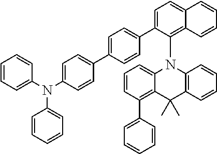 [SMILES: CC1(C)c2ccccc2N(c2c(-c3ccc(-c4ccc(N(c5ccccc5)c5ccccc5)cc4)cc3)ccc3ccccc23)c2cccc(-c3ccccc3)c21] | 75% |
| 591 | 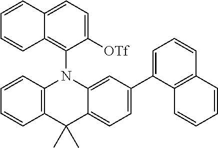 [SMILES: CC1(C)c2ccccc2N(c2c(OS(=O)(=O)C(F)(F)F)ccc3ccccc23)c2cc(-c3cccc4ccccc34)ccc21] | 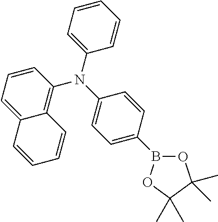 [SMILES: CC1(C)OB(c2ccc(N(c3ccccc3)c3cccc4ccccc34)cc2)OC1(C)C] | 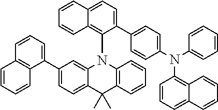 [SMILES: CC1(C)c2ccccc2N(c2c(-c3ccc(N(c4ccccc4)c4cccc5ccccc45)cc3)ccc3ccccc23)c2cc(-c3cccc4ccccc34)ccc21] | 73% |
| 593 | 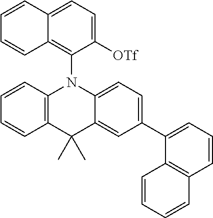 [SMILES: CC1(C)c2ccccc2N(c2c(OS(=O)(=O)C(F)(F)F)ccc3ccccc23)c2ccc(-c3cccc4ccccc34)cc21] | 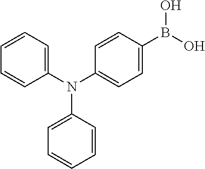 [SMILES: OB(O)c1ccc(N(c2ccccc2)c2ccccc2)cc1] | 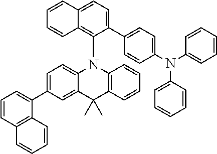 [SMILES: CC1(C)c2ccccc2N(c2c(-c3ccc(N(c4ccccc4)c4ccccc4)cc3)ccc3ccccc23)c2ccc(-c3cccc4ccccc34)cc21] | 74 % |
| 609 | 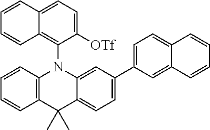 [SMILES: CC1(C)c2ccccc2N(c2c(OS(=O)(=O)C(F)(F)F)ccc3ccccc23)c2cc(-c3ccc4ccccc4c3)ccc21] | 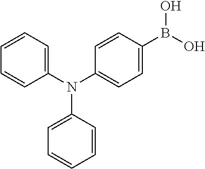 [SMILES: OB(O)c1ccc(N(c2ccccc2)c2ccccc2)cc1] | 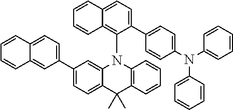 [SMILES: CC1(C)c2ccccc2N(c2c(-c3ccc(N(c4ccccc4)c4ccccc4)cc3)ccc3ccccc23)c2cc(-c3ccc4ccccc4c3)ccc21] | 80% |
| 615 | 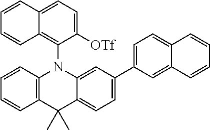 [SMILES: CC1(C)c2ccccc2N(c2c(OS(=O)(=O)C(F)(F)F)ccc3ccccc23)c2cc(-c3ccc4ccccc4c3)ccc21] | 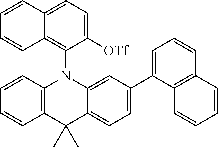 [SMILES: CC1(C)c2ccccc2N(c2c(OS(=O)(=O)C(F)(F)F)ccc3ccccc23)c2cc(-c3cccc4ccccc34)ccc21] | 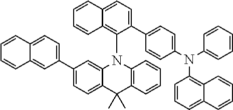 [SMILES: CC1(C)c2ccccc2N(c2c(-c3ccc(N(c4ccccc4)c4cccc5ccccc45)cc3)ccc3ccccc23)c2cc(-c3ccc4ccccc4c3)ccc21] | 78% |
| 625 | 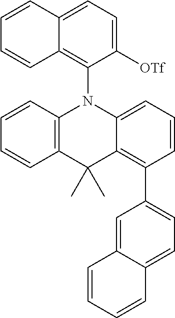 [SMILES: CC1(C)c2ccccc2N(c2c(OS(=O)(=O)C(F)(F)F)ccc3ccccc23)c2cccc(-c3ccc4ccccc4c3)c21] | 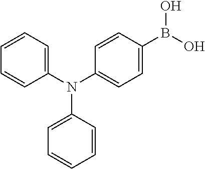 [SMILES: OB(O)c1ccc(N(c2ccccc2)c2ccccc2)cc1] | 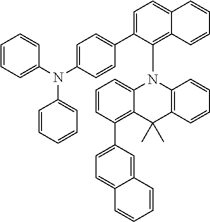 [SMILES: CC1(C)c2ccccc2N(c2c(-c3ccc(N(c4ccccc4)c4ccccc4)cc3)ccc3ccccc23)c2cccc(-c3ccc4ccccc4c3)c21] | 74% |
| 633 | 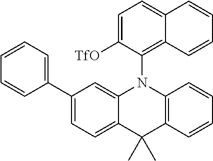 [SMILES: CC1(C)c2ccccc2N(c2c(OS(=O)(=O)C(F)(F)F)ccc3ccccc23)c2cc(-c3ccccc3)ccc21] | 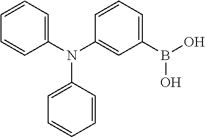 [SMILES: OB(O)c1cccc(N(c2ccccc2)c2ccccc2)c1] | 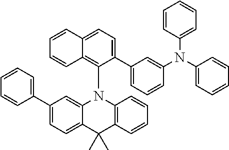 [SMILES: CC1(C)c2ccccc2N(c2c(-c3cccc(N(c4ccccc4)c4ccccc4)c3)ccc3ccccc23)c2cc(-c3ccccc3)ccc21] | 72% |

The remaining compounds other than the compounds described in Preparation Examples 1 to 4 and Tables 1 to 4 were also prepared in the same manner as in Preparation Examples described above, and the synthesis results are shown in Tables 5 and 6 below. Table 5 below is measurement values of ¹H NMR (CDCl₃, 300 MHz), and Table 6 below is measurement values of the FD-mass spectrometer (FD-MS: Field desorption mass spectrometry).

TABLE 5

| Compound | ¹H NMR(CDCl₃, 300 MHz) |
|---|---|
| 002 | δ = 8.07 (1H, d), 8.02 (1H, d), 7.78 (1H, d), 7.63 (1H, d), 7.54-7.41 (12H, |
| | m), 7.20 (2H, t), 7.08-7.02 (4H, m), 6.81-6.63 (7H, m), 6.55 (1H, d), 5.91 |
| | (1H, d), 5.69 (1H, s) 1.72 (6H, s) |
| 003 | δ = 8.07 (1H, d), 8.02 (1H, d), 7.78 (1H, d), 7.63 (1H, d), 7.54-7.41 (19H, |
| | m), 7.08-7.02 (4H, m), 6.80-6.69 (6H, m), 6.55 (1H, d), 5.91 (1H, d), 5.69 |
| | (1H, s), 1.72 (6H, s) |
| 005 | δ = 8.07 (1H, d), 8.02 (1H, d), 7.87 (1H, d), 7.78 (1H, d), 7.63-7.53 (2H, |
| | m), 7.55-7.51 (11H, m), 7.41-7.38 (3H, m), 7.28 (1H, t), 7.08-7.02 (4H, m), |
| | 6.80-6.69 (5H, m), 6.58-6.55 (2H, m), 5.91 (1H, d), 5.69 (1H, s), 1.72 (12H, |
| | s) |
| 009 | δ = 8.07 (1H, d), 8.02 (1H, d), 7.78 (1H, d), 7.63 (1H, d), 7.54-7.41 (16H, |
| | m), 7.16-7.05 (7H, m), 6.87-6.69 (6H, m), 6.55 (1H, d), 5.91 (1H, d), 5.69 |
| | (1H, s), 1.72 (6H, s) |
| 010 | δ = 8.07 (1H, d), 8.02 (1H, d), 7.87 (1H, d), 7.78 (1H, d), 7.63-7.53 (2H, |
| | m), 7.55-7.51 (8H, m), 7.41-7.38 (3H, m), 7.28 (1H, t), 7.16-7.05 (7H, m), |
| | 6.87-6.69 (5H, m), 6.58-6.55 (2H, m), 5.91 (1H, d), 5.69 (1H, s), 1.72 (12H, |
| | s) |
| 011 | δ = 8.07 (1H, d), 8.02 (1H, d), 7.87 (1H, d), 7.78 (1H, d), 7.63-7.62 (2H, |
| | m), 7.55-7.38 (13H, m), 7.28 (1H, t), 7.07-7.02 (4H, m), 6.89-6.88 (2H, m), |
| | 6.80-6.73 (3H, m), 6.59-6.55 (3H, m), 5.91 (1H, d), 5.69 (1H, s), 1.72 (12H, |
| | s) |
| 013 | δ = 8.07 (1H, d), 8.02 (1H, d), 7.87 (1H, d), 7.78 (1H, d), 7.63-7.51 (10H, |
| | m), 7.41-7.38 (3H, m), 7.28 (1H, t), 7.08-6.98 (5H, m), 6.80-6.73 (3H, m), |
| | 6.58-6.55 (2H, m), 5.91 (1H, d), 5.69 (1H, s), 1.72 (12H, s) |
| 014 | δ = 8.07 (1H, d), 8.02 (1H, d), 7.87 (1H, d), 7.78 (1H, d), 7.63-7.51 (7H, |
| | m), 7.38-7.02 (19H, m), 6.81-6.73 (4H, m), 6.63-6.55 (4H, m), 5.91 (1H, d), |
| | 5.69 (1H, s), 1.72 (6H, s) |
| 015 | δ = 8.07 (1H, d), 8.02 (1H, d), 7.87 (2H, d), 7.78-7.75 (2H, m), 7.63 (1H, |
| | d), 7.55-7.50 (7H, m), 7.41-7.02 (14H, m), 6.81-6.73 (3H, m), 6.63 (2H, d), |
| | 6.55 (2H, d), 6.39 (1H, d), 5.91 (1H, d), 5.69 (1H, s), 1.72 (6H, s) |
| 017 | δ = 8.93 (2H, d), 8.13-8.02 (5H, m), 7.88-7.78 (6H, m), 7.63 (1H, d), 7.54- |
| | 7.41 (12H, m), 7.08-7.02 (5H, m), 6.80-6.69 (4H, m), 6.55 (1H, d), 5.91 (1H, |
| | d), 5.69 (1H, s), 1.72 (6H, s) |
| 019 | δ = 8.07 (1H, d), 8.02 (1H, d), 7.89 (1H, d), 7.78 (1H, d), 7.66-7.63 (2H, |
| | m), 7.54-7.25 (15H, m), 7.08-7.02 (5H, m), 6.80-6.69 (4H, m), 6.55 (1H, d), |
| | 6.39 (1H, d), 5.91 (1H, d), 5.69 (1H, s), 1.72 (6H, s) |
| 038 | δ = 8.93 (2H, d), 8.12-8.02 (4H, m), 7.88-7.78 (5H, m), 7.63 (1H, d), 7.54- |
| | 7.41 (12H, m), 7.08-7.02 (4H, m), 6.91 (1H, s), 6.73-6.69 (3H, m), 6.55 (1H, |
| | d), 6.37 (1H, s), 6.30 (1H, d), 6.20 (1H, d), 1.72 (6H, s) |
| 040 | δ = 8.45 (1H, d), 8.07-7.98 (3H, m), 7.87-7.78 (3H, m), 7.63-7.62 (2H, m), |
| | 7.55-7.50 (7H, m), 7.41-7.38 (2H, m), 7.28-7.27 (2H, m), 7.08-7.02 (4H, m), |
| | 6.86 (1H, d), 6.75-6.73 (2H, m), 6.58-6.55 (2H, m), 6.37 (1H, s), 6.30 (1H, |
| | d), 6.20 (1H, d), 1.72 (12H, s) |
| 046 | δ = 8.07 (1H, d), 8.02 (1H, d), 7.87 (2H, d), 7.78 (1H, d), 7.63-7.62 (3H, |
| | m), 7.55-7.51 (6H, m), 7.41-7.38 (3H, m), 7.28 (2H, t), 7.08-7.02 (4H, m), |
| | 6.77-6.73 (4H, m), 6.58-6.55 (3H, m), 5.91 (2H, d), 1.72 (18H, s) |
| 053 | δ = 8.07 (2H, d), 8.02 (2H, d), 7.87 (1H, d), 7.78 (1H, d), 7.63-7.51 (10H, |
| | m), 7.41-7.38 (3H, m), 7.28 (1H, t), 7.08-6.98 (5H, m), 6.77-6.73 (3H, m), |
| | 6.58-6.55 (2H, m), 5.91 (2H, d), 1.72 (12H, s) |
| 064 | δ = 8.55 (1H, d), 8.42 (1H, d), 8.08-8.02 (4H, m), 7.87 (1H, d), 7.78 (1H, |
| | d), 7.63-7.53 (8H, m), 7.38 (1H, t), 7.28 (1H, t), 7.20 (2H, t), 7.05-7.02 |
| | (2H, m), 6.81-6.73 (4H, m), 6.65-6.55 (4H, m), 5.91 (1H, d), 5.69 (1H, s), |
| | 1.72 (6H, s) |
| 072 | δ = 8.55 (1H, d), 8.42 (1H, d), 8.08-8.02 (6H, m), 7.78 (1H, d), 7.63-7.51 |
| | (17H, m), 7.41 (1H, t), 7.05-7.02 (2H, m), 6.80-6.69 (6H, m), 6.55 (1H, d), |
| | 5.91 (1H, d), 5.69 (1H, s), 1.72 (6H, s) |
| 093 | δ = 8.55 (1H, d), 8.42 (1H, d), 8.08-8.02 (6H, m), 7.87 (1H, d), 7.78 (1H, |
| | d), 7.63-7.53 (11H, m), 7.38 (2H, t), 7.28 (1H, t), 7.05-6.98 (3H, m), 6.75- |
| | 6.73 (2H, m), 6.58-6.55 (2H, m), 6.37 (1H, s), 6.30 (1H, d), 6.20 (1H, d), |
| | 1.72 (12H, s) |
| 098 | δ = 8.93 (2H, d), 855 (1H, d), 8.42 (1H, d), 8.12-8.02 (6H, m), 7.88-7.78 |
| | (5H, m), 7.63-7.41 (13H, m), 7.05-7.02 (2H, m), 6.91 (1H, s), 6.73-6.69 (3H, |
| | m), 6.55 (1H, d), 6.37 (1H, s), 6.30 (1H, d), 6.20 (1H, d), 1.72 (6H, s) |
| 105 | δ = 8.55 (1H, d), 8.42 (1H, d), 8.08-8.02 (4H, m), 7.87 (1H, d), 7.78 (1H, |
| | d), 7.63-7.51 (14H, m), 7.41-7.38 (2H, m), 7.28 (1H, t), 7.05-7.02 (2H, m), |
| | 6.77-6.69 (5H, m), 6.58-6.55 (2H, m), 5.91 (2H, d), 1.72 (12H, s) |
| 109 | δ = 8.55 (1H, d), 8.42 (1H, d), 8.08-8.02 (4H, m), 7.78 (1H, d), 7.63-7.41 |
| | (17H, m), 7.16-7.02 (5H, m), 6.87 (1H, t), 6.77-6.69 (5H, m), 6.55 (1H, d), |
| | 5.91 (2H, d), 1.72 (6H, s) |
| 116 | δ = 8.55 (1H, d), 8.42 (1H, d), 8.08-8.02 (6H, m), 7.88-7.74 (5H, m), 7.63- |
| | 7.50 (11H, m), 7.38-7.36 (2H, m), 7.05-6.92 (3H, m), 6.77-6.73 (2H, m), 6.55 |
| | (1H, d), 5.91 (2H, d), 1.72 (6H, s) |
| 127 | δ = 8.07-8.00 (4H, m), 7.92-7.74 (7H, m), 7.63-7.41 (16H, m), 7.05-7.02 (2H, |
| | m), 6.80-6.69 (4H, m), 6.55 (1H, d), 5.91 (1H, d), 5.69 (1H, s), 1.72 (6H, s) |
| 131 | δ = 8.07-8.00 (4H, m), 7.92-7.87 (2H, m), 7.78-7.73 (2H, m), 7.63-7.38 (15H, |
| | m), 7.28 (1H, t), 7.05-7.02 (2H, m), 6.89-6.88 (2H, m), 6.80-6.73 (3H, m), |
| | 6.59-6.55 (3H, m), 5.91 (1H, d), 5.69 (1H, s), 1.72 (12H, s) |
| 143 | δ = 8.07-8.00 (4H, m), 7.92 (1H, d), 7.78-7.73 (2H, m), 7.63-7.41 (20H, m), |
| | 7.05-7.02 (2H, m), 6.73-6.69 (5H, m), 6.55 (1H, d), 6.37 (1H, s), 6.30 (1H, |
| | d), 6.20 (1H, d), 1.72 (6H, s) |
| 159 | δ = 8.07-7.89 (6H, m), 7.78-7.38 (19H, m), 7.07-7.02 (3H, m), 6.73-6.69 (3H, |
| | m), 6.55 (1H, d), 6.39-6.37 (2H, m), 6.30 (1H, d), 6.20 (1H, d), 1.72 (6H, s) |
| 164 | δ = 8.07-8.00 (4H, m), 7.92-7.87 (2H, m), 7.78-7.73 (2H, m), 7.63-7.53 (8H, |
| | m), 7.38 (1H, t), 7.28-7.20 (3H, m), 7.05-7.02 (2H, m), 6.81-6.73 (4H, m), |
| | 6.63-6.55 (4H, m), 5.91 (2H, d), 1.72 (12H, s) |
| 182 | δ = 8.93 (2H, d), 8.12-8.02 (4H, m), 7.93-7.78 (6H, m), 7.63 (1H, d), 7.54- |
| | 7.41 (9H, m), 7.20 (2H, t), 7.05-7.02 (2H, m), 6.81-6.63 (7H, m), 6.55 (1H, |
| | d), 5.91 (1H, d), 5.69 (1H, s), 1.72 (6H, s) |
| 199 | δ = 8.07 (1H, d), 8.02 (1H, d), 7.89-7.87 (4H, m), 7.66-7.63 (2H, m), 7.54- |
| | 7.53 (4H, m), 7.38-7.32 (3H, m), 7.20 (4H, t), 7.38 (1H, t), 7.11-7.05 (2H, |
| | m), 6.81-6.63 (11H, m), 6.55 (1H, d), 1.72 (6H, s) |
| 202 | δ = 8.07 (1H, d), 8.02 (1H, d), 7.89-7.78 (4H, m), 7.66-7.63 (2H, m), 7.54- |
| | 7.32 (12H, m), 7.20 (2H, t), 7.05-7.02 (2H, m), 6.81-6.63 (6H, m), 6.55 (1H, |
| | d), 6.37 (1H, s), 6.30 (1H, d), 6.20 (1H, d), 1.72 (6H, s) |
| 224 | δ = 8.45-8.41 (2H, m), 8.20 (1H, d), 8.07-7.98 (3H, m), 7.78 (1H, d), 7.63- |
| | 7.44 (7H, m), 7.26-7.20 (5H, m), 7.07-7.02 (3H, m), 6.89-6.73 (5H, m), 6.63- |
| | 6.51 (6H, m), 1.72 (6H, s) |
| 243 | δ = 8.07 (1H, d), 8.02 (1H, d), 7.87 (1H, d), 7.78 (1H, d), 7.63-7.62 (2H, |
| | m), 7.55-7.51 (7H, m), 7.41-7.38 (2H, m), 7.28-7.20 (3H, m), 7.11-7.02 (5H, |
| | m), 6.75-6.55 (11H, m), 1.72 (12H, s) |
| 249 | δ = 8.07 (1H, d), 8.02 (1H, d), 7.78 (1H, d), 7.63-7.51 (8H, m), 7.41-7.36 |
| | (2H, m), 7.20 (4H, t), 7.08-7.02 (4H, m), 6.81-6.55 (11H, m), 1.72 (6H, s) |
| 273 | δ = 8.55 (1H, d), 8.42 (1H, d), 8.08-8.02 (4H, m), 7.78 (2H, d), 7.63-7.53 |
| | (9H, m), 7.36 (1H, d), 7.20 (4H, t), 7.05-7.02 (2H, m), 6.81-6.55 (11H, m), |
| | 1.72 (6H, s) |
| 287 | δ = 8.55 (1H, d), 8.42 (1H, d), 8.08-8.02 (4H, m), 7.78 (1H, d), 7.63-7.53 |
| | (11H, m), 7.38 (1H, t), 7.26-7.20 (3H, m), 7.07-6.98 (4H, m), 6.81-6.63 (6H, |
| | m), 6.55-6.51 (2H, m), 1.72 (6H, s) |
| 298 | δ = 8.07-8.00 (4H, m), 7.92 (1H, d), 7.78-7.73 (2H, m), 7.63-7.51 (15H, m), |
| | 7.41-7.36 (2H, m), 7.20 (2H, t), 7.05-7.02 (2H, m), 6.81-6.55 (10H, m), 1.72 |
| | (6H, s) |
| 305 | δ = 8.07-7.92 (5H, m), 7.78-7.73 (2H, m), 7.63-7.53 (8H, m), 7.26-7.20 (5H, |
| | m), 7.07-7.02 (3H, m), 6.81-6.69 (9H, m), 6.55-6.51 (2H, m), 1.72 (6H, s) |
| 312 | δ = 8.07-7.73 (13H, m), 7.63-7.49 (13H, m), 7.38-7.36 (2H, m), 7.26 (1H, t), |
| | 7.07-6.98 (4H, m), 6.73-6.69 (3H, m), 6.55-6.51 (2H, m), 1.72 (6H, s) |
| 319 | δ = 8.07-8.02 (4H, m), 7.78 (1H, d), 7.63-7.41 (11H, m), 7.20 (2H, t), 7.11- |
| | 6.98 (6H, m), 6.89-6.73 (6H, m), 6.63-6.55 (4H, m), 1.72 (6H, s) |
| 322 | δ = 7.90 (1H, d), 7.84 (1H, d), 7.54-7.41 (13H, m), 7.30-7.20 (4H, m), 7.11- |
| | 7.02 (4H, m), 6.81-6.63 (8H, m), 6.55 (1H, d), 1.72 (6H, s) |
| 325 | δ = = 7.90 (1H, d), 7.84 (1H, d), 7.62 (1H, d), 7.55-7.26 (18H, m), 7.11-7.02 |
| | (4H, m), 6.81 (1H, s), 6.75-6.69 (5H, m), 6.58-6.55 (2H, m), 1.72 (12H, s) |
| 343 | δ = = 7.90 (1H, d), 7.84 (1H, d), 7.61 (1H, s), 7.54-7.30 (23H, m), 7.05-7.02 |
| | (3H, m), 6.73-6.69 (5H, m), 6.61 (1H, d), 6.55 (1H, d), 1.72 (6H, s) |
| 352 | δ = 8.55 (1H, d), 8.42 (1H, d), 8.08-8.04 (2H, m), 7.90 (1H, d), 7.84 (1H, |
| | d), 7.61-7.26 (22H, m), 7.05-7.02 (3H, m), 6.73-6.69 (5H, m), 6.61 (1H, d), |
| | 6.55 (1H, d), 1.72 (6H, s) |
| 367 | δ = 7.90-7.74 (6H, m), 7.54-7.26 (19H, m), 7.07-7.02 (4H, m), 6.73-6.69 (3H, |
| | m), 6.55 (1H, d), 6.51 (1H, d), 1.72 (6H, s) |
| 384 | δ = 8.55 (1H, d), 8.42 (1H, d), 8.08 (1H, d), 8.04 (1H, d), 7.90-7.84 (3H, |
| | m), 7.62-7.55 (5H, m), 7.45-7.20 (7H, m), 7.11-7.02 (4H, m), 6.81-6.73 (5H, |
| | m), 6.63-6.55 (4H, m), 1.72 (12H, s) |
| 393 | δ = 8.55 (1H, d), 8.42 (1H, d), 8.08-8.02 (4H, m), 7.90-7.84 (3H, m), 7.62- |
| | 7.53 (8H, m), 7.45-7.26 (6H, m), 7.11-7.68 (5H, m), 6.81 (1H, s), 6.75-6.73 |
| | (3H, m), 6.58-6.55 (2H, m), 1.72 (12H, s) |
| 397 | δ = 8.93 (2H, d), 8.55 (1H, d), 8.42 (1H, d), 8.13-8.04 (5H, m), 7.90-7.82 |
| | (7H, m), 7.61-7.41 (11H, m), 7.30-7.26 (2H, m), 7.11-7.02 (5H, m), 6.81 (1H, |
| | s), 6.73-6.69 (4H, m), 6.55 (1H, d), 1.72 (6H, s) |
| 402 | δ = 8.55 (1H, d), 8.42 (1H, d), 8.08 (1H, d), 8.04 (1H, d), 7.90 (1H, d), |
| | 7.84 (1H, d), 7.61-7.20 (17H, m), 7.05-7.02 (3H, m), 6.73-6.61 (8H, m), 1.72 |
| | (6H, s) |
| 419 | δ = 8.55 (1H, d), 8.42 (1H, d), 8.08 (1H, d), 8.04 (1H, d), 7.90-7.84 (3H, |
| | m), 7.66-7.25 (19H, m), 7.07-7.02 (4H, m), 6.73-6.69 (3H, m), 6.61-6.55 (2H, |
| | m), 6.39 (1H, d), 1.72 (6H, s) |
| 425 | δ = 8.55 (1H, d), 8.42 (1H, d), 8.08 (1H, d), 8.04 (1H, d), 7.90-7.84 (3H, |
| | m), 7.62-7.26 (18H, m), 7.07-7.02 (4H, m), 6.75-6.69 (4H, m), 6.58-6.51 (3H, |
| | m), 1.72 (12H, s) |
| 431 | δ = 8.55 (1H, d), 8.42 (1H, d), 8.08 (1H, d), 8.04 (1H, d), 7.90-7.84 (3H, |
| | m), 7.62-7.26 (17H, m), 7.07-7.02 (4H, m), 6.89-6.88 (2H, m), 6.75-6.73 (2H, |
| | m), 6.59-6.51 (4H, m), 1.72 (12H, s) |
| 444 | δ = 8.00 (2H, d), 7.92-7.84 (4H, m), 7.73 (1H, d), 7.62-7.55 (5H, m), 7.45- |
| | 7.20 (7H, m), 7.11-7.02 (4H, m), 6.81-6.73 (5H, m), 6.63-6.55 (4H, m), 1.72 |
| | (12H, s) |
| 458 | δ = 8.93 (2H, d), 8.12 (2H, d), 8.00-7.82 (9H, m), 7.73 (1H, d), 7.59- |
| | 7.41(11H, m), 7.30-7.26 (2H, m), 7.11-7.02 (4H, m), 6.91 (1H, s), 6.81 (1H, |
| | s), 6.73-6.69 (4H, m), 6.55 (1H, d), 1.72 (6H, s) |
| 467 | δ = 8.00-7.73 (10H, m), 7.61-7.26 (18H, m), 7.05-7.02 (3H, m), 6.73-6.69 (3H, |
| | m), 6.61 (1H, d), 6.55 (1H, d), 1.72 (6H, s) |
| 481 | δ = 8.00 (2H, d), 7.92-7.84 (3H, m), 7.73 (1H, d), 7.59-7.58 (3H, m), 7.45 |
| | (1H, t), 7.30-7.20 (7H, m), 7.07-7.02 (4H, m), 6.81-6.73 (3H, m), 6.63 (4H, |
| | d), 6.55-6.51 (2H, m), 1.72 (6H, s) |
| 493 | δ = 8.07-8.00 (4H, m), 7.92-7.84 (4H, m), 7.73 (1H, d), 7.62-7.53 (8H, m), |
| | 7.45-7.26 (7H, m), 7.07-6.98 (5H, m), 6.75-6.73 (2H, m), 6.59-6.51 (3H, m), |
| | 1.72 (12H, s) |
| 506 | δ = 8.93 (2H, d), 8.12 (2H, d), 7.93-7.82 (7H, m), 7.61 (1H, s), 7.45 (1H, |
| | t), 7.36-7.20 (7H, m), 7.05-7.02 (3H, m), 6.81-6.73 (3H, m), 6.63-6.55 (6H, |
| | m), 1.72 (6H, s) |
| 515 | δ = 8.93 (2H, d), 8.12 (2H, d), 7.93-7.77 (11H, m), 7.50-7.20 (9H, m), 7.07- |
| | 7.02 (4H, m), 6.81-6.73 (2H, m), 6.63 (2H, d), 6.55-6.51 (2H, m), 1.72 (6H, |
| | s) |
| 531 | δ = 8.45 (1H, d), 8.41 (1H, d), 8.20 (1H, d), 7.98 (1H, d), 7.90 (1H, d), |
| | 7.84 (1H, d), 7.58-7.45 (4H, m), 7.30-7.20 (6H, m), 7.11-7.02 (4H, m), 6.81- |
| | 6.73 (5H, m), 6.63 (4H, d), 6.55 (1H, d), 1.72 (6H, s) |
| 540 | δ = 8.45 (1H, d), 8.41 (1H, d), 8.20 (1H, d), 8.07-7.98 (3H, m), 7.90-7.84 |
| | (2H, m), 7.61-7.20 (14H, m), 7.05-6.98 (4H, m), 6.81-6.73 (2H, m), 6.63-6.55 |
| | (4H, m), 1.72 (6H, s) |
| 546 | δ = 7.93-7.84 (4H, m), 7.77 (1H, d), 7.63 (1H, d), 7.55 (1H, d), 7.45-7.20 |
| | (9H, m), 7.11-7.02 (4H, m), 6.81-6.73 (5H, m), 6.63 (4H, d), 6.55 (1H, d), |
| | 1.72 (12H, s) |
| 553 | δ = 7.93-7.84 (5H, m), 7.77 (1H, s), 7.63-7.61 (3H, m), 7.55 (1H, d), 7.45- |
| | 7.20 (10H, m), 7.05-7.02 (3H, m), 6.81 (1H, t), 6.75-6.73 (2H, m), 6.63-6.55 |
| | (5H, m), 1.72 (18H, s) |
| 562 | δ = 8.04 (1H, d), 8.02 (1H, d), 7.78 (1H, d), 7.63 (1H, d), 7.54-7.41 (16H, |
| | m), 7.20 (2H, t), 7.11-7.02 (3H, m), 6.81-6.63 (10H, m), 6.55 (1H, d), 1.72 |
| | (6H, s) |
| 580 | δ = 8.07 (1H, d), 8.02 (1H, d), 7.78 (1H, d), 7.63 (1H, d), 7.54-7.41 (9H, |
| | m), 7.26-7.20 (9H, m), 7.07-7.02 (3H, m), 6.81-6.63 (9H, m), 6.57-6.55 (2H, |
| | m), 1.72 (6H, s) |
| 591 | δ = 8.55 (1H, d), 8.42 (1H, d), 8.08-8.02 (6H, m), 7.78 (1H, d), 7.63-7.53 |
| | (11H, m), 7.38 (1H, t), 7.20 (2H, t), 7.11-6.98 (4H, m), 6.81-6.63 (8H, m), |
| | 6.55 (1H, d), 1.72 (6H, s) |
| 593 | δ = 8.55 (1H, d), 8.42 (1H, d), 8.08-8.02 (4H, m), 7.78 (1H, d), 7.63-7.53 |
| | (9H, m), 7.36 (1H, d), 6.20 (4H, t), 7.05-7.02 (2H, m), 6.81-6.61 (11H, m), |
| | 1.72 (6H, s) |
| 609 | δ = 8.07-7.92 (5H, m), 7.78-7.73 (2H, m), 7.63-7.53 (8H, m), 7.20 (4H, t), |
| | 7.11-7.02 (3H, m), 6.81-6.63 (11H, m), 6.55 (1H, d), 1.72 (6H, s) |
| 615 | δ = 8.07-7.92 (7H, m), 7.78-7.73 (2H, m), 7.63-7.53 (11H, m), 7.38 (1H, t), |
| | 7.20 (2H, t), 7.11-6.98 (4H, m), 6.81-6.63 (8H, m), 6.55 (1H, d), 1.72 (6H, |
| | s) |
| 625 | δ = 8.07-7.92 (5H, m), 7.78-7.73 (2H, m), 7.63-7.53 (8H, m), 7.26-7.20 (5H, |
| | m), 7.07-7.02 (3H, m), 6.81-6.63 (9H, m), 6.57-6.55 (2H, m), 1.72 (6H, s) |
| 633 | δ = 8.07 (1H, d), 8.02 (1H, d), 7.78 (1H, d), 7.63 (1H, d), 7.54-7.41 (8H, |
| | m), 7.20 (4H, t), 7.11-7.02 (3H, m), 6.89-6.73 (7H, m), 6.63-6.55 (6H, |
| | m), .172 (6H, s) |

TABLE 6

| Compound | FD-MS | Compound | FD-MS |
|---|---|---|---|
| 002 | m/z = 654.84 (C₄₉H₃₈N₂ = 654.30) | 003 | m/z = 730.94 (C₅₅H₄₂N₂ = 730.33) |
| 005 | m/z = 771.00 (C₅₈H₄₆N₂ = 770.37) | 009 | m/z = 730.94 (C₅₅H₄₂N₂ = 730.33) |
| 010 | m/z = 771.00 (C₅₈H₄₆N₂ = 770.37) | 011 | m/z = 771.00 (C₅₈H₄₆N₂ = 770.37) |
| 013 | m/z = 744.96 (C₅₆H₄₄N₂ = 744.35) | 014 | m/z = 819.04 (C₆₂H₄₆N₂ = 818.37) |
| 015 | m/z = 817.03 (C₆₂H₄₄N₂ = 816.35) | 017 | m/z = 805.02 (C₆₁H₄₄N₂ = 804.35) |
| 019 | m/z = 744.92 (C₅₅H₄₀N₂O = 744.31) | 038 | m/z = 754.96 (C₅₇H₄₂N₂ = 754.33) |
| 040 | m/z = 801.05 (C₅₈H₄₄N₂ = 800.32) | 046 | m/z = 811.06 (C₆₁H₅₀N₂ = 810.40) |
| 053 | m/z = 744.96 (C₅₆H₄₄N₂ = 744.35) | 064 | m/z = 744.96 (C₅₆H₄₄N₂ = 744.35) |
| 072 | m/z = 831.05 (C₆₃H₄₆N₂ = 830.37) | 093 | m/z = 795.02 (C₆₀H₄₆N₂ = 794.37) |
| 098 | m/z = 805.02 (C₆₁H₄₄N₂ = 804.35) | 105 | m/z = 821.06 (C₆₂H₄₈N₂ = 820.38) |
| 109 | m/z = 780.99 (C₅₉H₄₄N₂ = 780.35) | 116 | m/z = 728.92 (C₅₅H₄₀N₂ = 728.32) |
| 127 | m/z = 754.96 (C₅₇H₄₂N₂ = 754.33) | 131 | m/z = 821.06 (C₆₂H₄₈N₂ = 820.38) |
| 143 | m/z = 780.99 (C₅₉H₄₄N₂ = 780.35) | 159 | m/z = 794.98 (C₅₉H₄₂N₂O = 794.33) |
| 164 | m/z = 744.96 (C₅₆H₄₄N₂ = 744.35) | 182 | m/z = 754.96 (C₅₇H₄₂N₂ = 754.33) |
| 199 | m/z = 744.92 (C₅₅H₄₀N₂O = 744.31) | 202 | m/z = 744.92 (C₅₅H₄₀N₂O = 744.31) |
| 224 | m/z = 760.98 (C₅₅H₄₀N₂S = 760.29) | 243 | m/z = 771.00 (C₅₈H₄₆N₂ = 770.37) |
| 249 | m/z = 654.84 (C₄₉H₃₈N₂ = 654.30) | 273 | m/z = 704.90 (C₅₃H₄₀N₂ = 704.32) |
| 287 | m/z = 754.96 (C₅₇H₄₂N₂ = 754.33) | 298 | m/z = 780.99 (C₅₉H₄₄N₂ = 780.35) |
| 305 | m/z = 704.90 (C₅₃H₄₀N₂ = 704.32) | 312 | m/z = 805.02 (C₆₁H₄₄N₂ = 804.35) |
| 319 | m/z = 704.90 (C₅₃H₄₀N₂ = 704.32) | 322 | m/z = 654.84 (C₄₉H₃₈N₂ = 654.30) |
| 325 | m/z = 771.00 (C₅₈H₄₆N₂ = 770.37) | 343 | m/z = 730.94 (C₅₅H₄₂N₂ = 730.33) |
| 352 | m/z = 780.99 (C₅₉H₄₄N₂ = 780.35) | 367 | m/z = 704.90 (C₅₃H₄₀N₂ = 704.32) |
| 384 | m/z = 744.96 (C₅₆H₄₄N₂ = 744.35) | 393 | m/z = 795.02 (C₆₀H₄₆N₂ = 794.37) |
| 397 | m/z = 855.07 (C₆₅H₄₆N₂ = 854.37) | 402 | m/z = 704.90 (C₅₃H₄₀N₂ = 704.32) |
| 419 | m/z = 794.98 (C₅₉H₄₂N₂O = 794.33) | 425 | m/z = 821.06 (C₆₂H₄₈N₂ = 820.38) |
| 431 | m/z = 821.06 (C₆₂H₄₈N₂ = 820.38) | 444 | m/z = 744.96 (C₅₆H₄₄N₂ = 744.35) |
| 458 | m/z = 805.02 (C₆₁H₄₄N₂ = 804.35) | 467 | m/z = 754.96 (C₅₇H₄₂N₂ = 754.33) |
| 481 | m/z = 628.80 (C₄₇H₃₆N₂ = 628.29) | 493 | m/z = 795.02 (C₆₀H₄₆N₂ = 794.37) |
| 506 | m/z = 728.92 (C₅₅H₄₀N₂ = 728.32) | 515 | m/z = 728.92 (C₅₅H₄₀N₂ = 728.32) |
| 531 | m/z = 684.89 (C₄₉H₃₆N₂S = 684.26) | 540 | m/z = 734.95 (C₅₃H₃₈N₂S = 734.28) |
| 546 | m/z = 694.90 (C₅₂H₄₂N₂ = 694.33) | 553 | m/z = 811.06 (C₆₁H₅₀N₂ = 810.40) |
| 562 | m/z = 730.94 (C₅₅H₄₂N₂ = 730.33) | 580 | m/z = 730.94 (C₅₅H₄₂N₂ = 730.33) |
| 591 | m/z = 754.96 (C₅₇H₄₂N₂ = 754.33) | 593 | m/z = 704.90 (C₅₃H₄₀N₂ = 704.32) |
| 609 | m/z = 704.90 (C₅₃H₄₀N₂ = 704.32) | 615 | m/z = 704.90 (C₅₃H₄₀N₂ = 704.32) |
| 625 | m/z = 704.90 (C₅₃H₄₀N₂ = 704.32) | 633 | m/z = 654.84 (C₄₉H₃₈N₂ = 654.30) |

### Experimental Examples

### Experimental Example 1

### Experimental Example 1-1. Manufacture of Organic Light Emitting Devices

A glass substrates coated with a thin film of ITO to a thickness of 1,500 Å was washed with distilled water and ultrasonic waves. After washing the substrate with distilled water, ultrasonic cleaning was performed on the washed substrates with a solvent such as acetone, methanol, isopropyl alcohol, or the like, and the ultrasonic cleaned substrates were dried, and UVO-treated for 5 minutes using UV in a UV cleaner. Thereafter, after transferring the substrate to a plasma cleaner (PT), plasma treatment was performed in order to increase the ITO work function and remove the residual film in a vacuum state, and then the substrates were transferred to thermal deposition equipment for organic deposition.

Subsequently, after performing air exhaust until the degree of vacuum in the chamber reached 10⁻⁶ torr, an electric current was applied to the cell to evaporate 2-TNATA so that a hole injection layer having a thickness of 600 Å was deposited on the ITO substrates. N,N′-bis(α-naphthyl)-N,N′-diphenyl-4,4′-diamine (NPB) below was put in another cell within vacuum deposition equipment, and an electric current was applied to the cell to evaporate it so that a hole transport layer with a thickness of 300 Å was deposited on the hole injection layer.

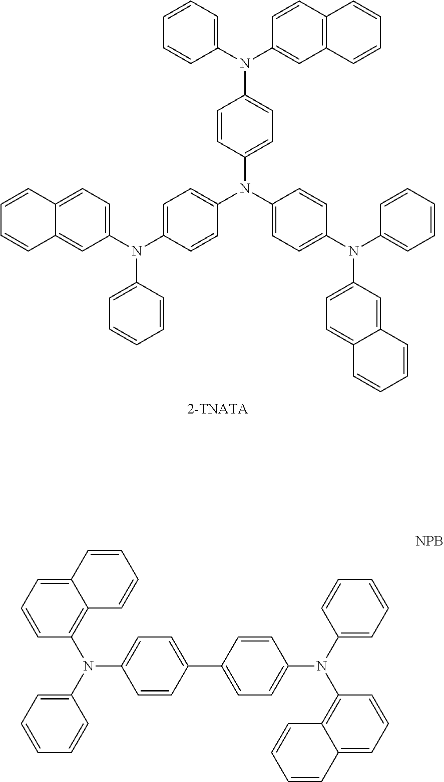 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1]

A light emitting layer was thermal vacuum deposited thereon as follows. A compound of 9-[4-(4,6-diphenyl-1,3,5-triazin-2-yl)phenyl]-9′-phenyl-3,3′-bi-9H-carbazole was deposited to a thickness of 400 Å as a host, and the host was doped with a green phosphorescent dopant Ir(ppy)₃ at 7% based on the weight of the host material to deposit the light emitting layer. Thereafter, bathocuproine (BCP) was deposited to a thickness of 60 Å as a hole blocking layer, and Alq₃ was deposited to a thickness of 200 Å thereon as an electron transport layer. Finally, after lithium fluoride (LiF)) was deposited to a thickness of 10 Å on the electron transport layer to form an electron injection layer, negative electrode were manufactured by depositing aluminum (Al) to a thickness of 1,200 Å on the electron injection layer, thereby forming an organic electroluminescent devices.

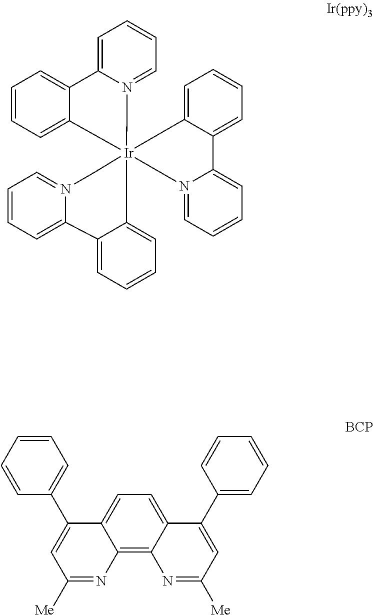

Meanwhile, all organic compounds required to manufacture the OLED devices were vacuum sublimated and purified under 10⁻⁶ to 10⁻⁸ torr for each material respectively, and used for OLED fabrication.

At this time, the comparative compounds used in the hole transport layers of Comparative Examples below are as follows.

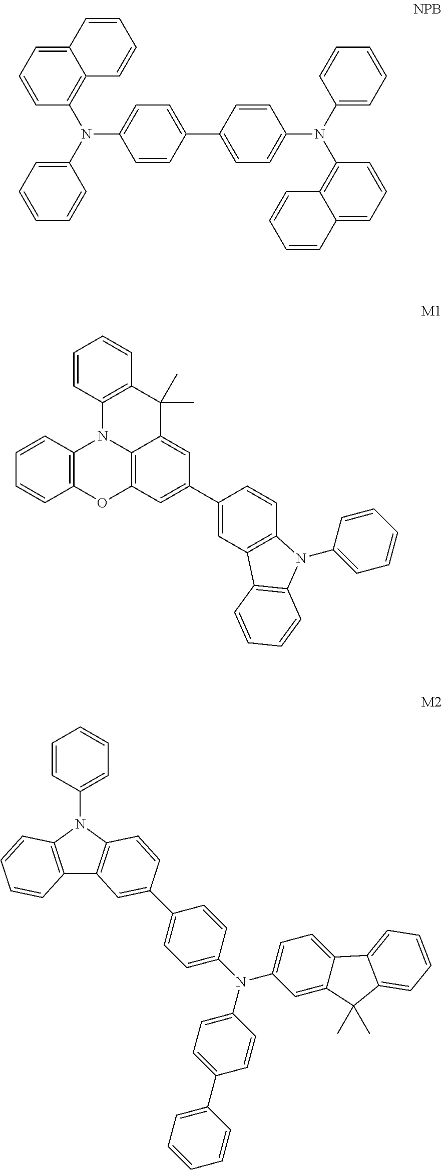 [SMILES: c1ccc(N(c2ccc(-c3ccc(N(c4ccccc4)c4cccc5ccccc45)cc3)cc2)c2cccc3ccccc23)cc1 ; CC1(C)c2ccccc2N2c3ccccc3Oc3cc(-c4ccc5c(c4)c4ccccc4n5-c4ccccc4)cc1c32 ; CC1(C)c2ccccc2-c2ccc(N(c3ccc(-c4ccccc4)cc3)c3ccc(-c4ccc5c(c4)c4ccccc4n5-c4ccccc4)cc3)cc21]

### Experimental Example 1-2. Driving Voltages and Luminous Efficiencies of Organic Light Emitting Devices

Electroluminescence (EL) properties were measured for the organic electroluminescent devices fabricated as described above with M7000 of McScience Inc., and T₉₀ values were measured with the measurement results when the reference luminance was 6,000 cd/m² through the device lifetime measurement system (M6000) manufactured by McScience Inc.

The properties of the organic electroluminescent devices according to the present disclosure are as shown in Table 7 below.

TABLE 7

| | | Driving | Luminous | |
|---|---|---|---|---|
| | | voltage | efficiency | Lifetime |
| | Compound | (V) | (cd/A) | (T₉₀) |
| Example 1 | 002 | 4.11 | 120.42 | 136 |
| Example 2 | 003 | 4.31 | 118.44 | 125 |
| Example 3 | 005 | 4.16 | 123.94 | 139 |
| Example 4 | 009 | 4.29 | 122.31 | 144 |
| Example 5 | 010 | 4.14 | 114.53 | 140 |
| Example 6 | 011 | 4.09 | 116.38 | 136 |
| Example 7 | 013 | 4.05 | 117.32 | 129 |
| Example 8 | 014 | 4.17 | 115.91 | 136 |
| Example 9 | 015 | 4.25 | 120.05 | 139 |
| Example 10 | 017 | 4.05 | 117.33 | 141 |
| Example 11 | 019 | 4.00 | 118.95 | 142 |
| Example 12 | 038 | 4.12 | 120.35 | 139 |
| Example 13 | 040 | 3.99 | 118.59 | 125 |
| Example 14 | 046 | 3.96 | 114.41 | 142 |
| Example 15 | 053 | 3.95 | 120.17 | 138 |
| Example 16 | 064 | 4.07 | 120.11 | 140 |
| Example 17 | 072 | 4.17 | 121.09 | 128 |
| Example 18 | 093 | 4.01 | 120.10 | 135 |
| Example 19 | 098 | 3.94 | 118.77 | 132 |
| Example 20 | 105 | 4.03 | 119.76 | 139 |
| Example 21 | 109 | 4.11 | 116.36 | 135 |
| Example 22 | 116 | 4.06 | 117.96 | 136 |
| Example 23 | 127 | 3.98 | 121.31 | 142 |
| Example 24 | 131 | 4.03 | 120.55 | 137 |
| Example 25 | 143 | 4.15 | 119.21 | 135 |
| Example 26 | 159 | 4.17 | 120.29 | 140 |
| Example 27 | 164 | 4.05 | 121.67 | 136 |
| Example 28 | 182 | 3.99 | 120.75 | 124 |
| Example 29 | 199 | 3.96 | 114.53 | 136 |
| Example 30 | 202 | 4.12 | 115.35 | 131 |
| Example 31 | 224 | 3.97 | 113.15 | 134 |
| Example 32 | 243 | 4.16 | 121.19 | 141 |
| Example 33 | 249 | 4.07 | 116.54 | 143 |
| Example 34 | 273 | 4.13 | 114.85 | 130 |
| Example 35 | 287 | 3.93 | 115.69 | 136 |
| Example 36 | 298 | 4.18 | 116.14 | 140 |
| Example 37 | 305 | 3.96 | 117.51 | 131 |
| Example 38 | 312 | 4.06 | 120.36 | 136 |
| Example 39 | 319 | 4.08 | 115.35 | 124 |
| Example 40 | 322 | 3.92 | 117.38 | 142 |
| Example 41 | 325 | 4.11 | 114.31 | 140 |
| Example 42 | 343 | 4.07 | 113.34 | 133 |
| Example 43 | 352 | 3.95 | 112.46 | 141 |
| Example 44 | 367 | 3.92 | 120.03 | 130 |
| Example 45 | 384 | 4.11 | 113.47 | 134 |
| Example 46 | 393 | 3.95 | 120.02 | 131 |
| Example 47 | 397 | 3.94 | 117.59 | 142 |
| Example 48 | 402 | 4.09 | 117.30 | 136 |
| Example 49 | 419 | 4.22 | 118.33 | 133 |
| Example 50 | 425 | 3.96 | 115.68 | 139 |
| Example 51 | 431 | 4.13 | 118.17 | 142 |
| Example 52 | 444 | 4.13 | 116.74 | 141 |
| Example 53 | 458 | 4.05 | 114.11 | 133 |
| Example 54 | 467 | 3.96 | 113.84 | 139 |
| Example 55 | 481 | 4.06 | 120.03 | 136 |
| Example 56 | 493 | 4.21 | 120.33 | 128 |
| Example 57 | 506 | 4.05 | 112.58 | 125 |
| Example 58 | 515 | 3.98 | 116.35 | 137 |
| Example 59 | 531 | 4.07 | 118.14 | 141 |
| Example 60 | 540 | 4.16 | 120.94 | 137 |
| Example 61 | 546 | 4.13 | 118.25 | 125 |
| Example 62 | 553 | 4.07 | 117.79 | 126 |
| Example 63 | 562 | 3.95 | 114.81 | 138 |
| Example 64 | 580 | 3.94 | 110.06 | 134 |
| Example 65 | 591 | 4.16 | 117.91 | 139 |
| Example 66 | 593 | 3.96 | 113.71 | 143 |
| Example 67 | 609 | 4.17 | 115.25 | 135 |
| Example 68 | 615 | 3.91 | 120.03 | 130 |
| Example 69 | 625 | 3.94 | 117.96 | 138 |
| Example 70 | 633 | 4.09 | 119.72 | 133 |
| Comparative | NPB | 4.55 | 101.01 | 113 |
| Example 1 | | | | |
| Comparative | M1 | 5.83 | 82.11 | 97 |
| Example 2 | | | | |
| Comparative | M2 | 4.40 | 103.53 | 120 |
| Example 3 | | | | |

It could be confirmed that the organic light emitting devices of Examples 1 to 70 according to an embodiment of the present disclosure had low driving voltages and excellent efficiencies and lifetimes compared to the organic light emitting devices of Comparative Examples 1 to 3.

### Experimental Example 2

### Experimental Example 2-1. Manufacture of Organic Light Emitting Devices

The transparent electrode ITO thin films obtained from glass for OLED (manufactured by Samsung Corning) were each ultrasonically washed for 5 minutes using trichloroethylene, acetone, ethanol, and distilled water sequentially, and then put and stored in isopropanol and used. Next, the ITO substrates were installed on the substrate folder of the vacuum deposition equipment, and 4,4′,4″-tris(N,N-(2-naphthyl)-phenylamino)triphenylamine (2-TNATA) below was put in the cell within the vacuum deposition equipment.

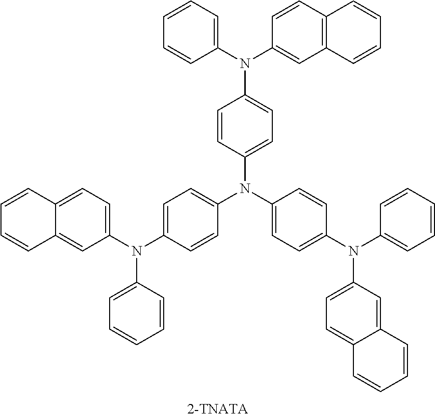 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, after performing air exhaust until the degree of vacuum in the chamber reached 10⁻⁶ torr, an electric current was applied to the cell to evaporate 2-TNATA so that a hole injection layer having a thickness of 600 Å was deposited on the ITO substrates. N,N′-bis(α-naphthyl)-N,N′-diphenyl-4,4′-diamine (NPB) below was put in another cell within the vacuum deposition equipment, and an electric current was applied to the cell to evaporate it so that a hole transport layer with a thickness of 150 Å was deposited on the hole injection layer.

The compounds shown in Table 8 below were deposited to a thickness of 50 Å on the hole transport layer to form an electron blocking layer.

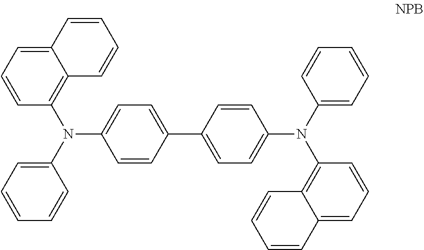 [SMILES: c1ccc(N(c2ccc(-c3ccc(N(c4ccccc4)c4cccc5ccccc45)cc3)cc2)c2cccc3ccccc23)cc1]

After forming the hole injection layer, the hole transport layer, and the electron blocking layer in this way, a blue light emitting material having the structure below was deposited as a light emitting layer thereon. Specifically, H1, a blue light-emitting host material, was vacuum-deposited to a thickness of 200 Å on one cell within the vacuum deposition equipment, and D1, a blue light-emitting dopant material, was vacuum-deposited thereon at 5% based on the weight of the host material.

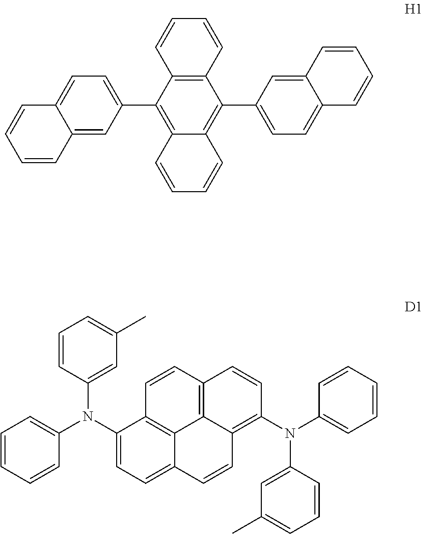

Subsequently, a compound of the structural formula E1 below was deposited to a thickness of 300 Å as an electron transport layer.

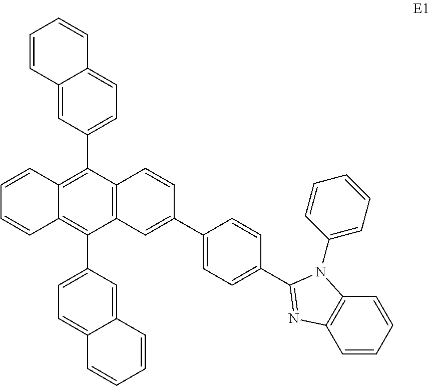 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

Lithium fluoride (LiF) was deposited to a thickness of 10 Å as an electron injection layer, and aluminum (Al) was deposited to a thickness of 1,000 Å to fabricate OLED devices.

Meanwhile, all organic compounds required for fabricating the OLED devices were vacuum sublimated and purified under 10⁻⁶ to 10⁻⁸ torr for each material respectively, and used for OLED fabrication.

At this time, the comparative compounds used in the electron blocking layers of Comparative Examples below are as follows.

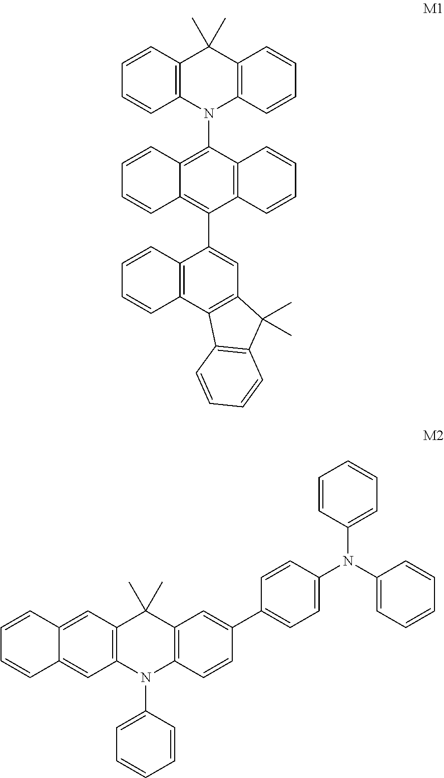 [SMILES: CC1(C)c2ccccc2N(c2c3ccccc3c(-c3cc4c(c5ccccc35)-c3ccccc3C4(C)C)c3ccccc23)c2ccccc21 ; CC1(C)c2cc(-c3ccc(N(c4ccccc4)c4ccccc4)cc3)ccc2N(c2ccccc2)c2cc3ccccc3cc21]

Experimental Example 2-2. Driving voltages and Luminous efficiencies of Organic Light Emitting Devices

Electroluminescence (EL) properties were measured for the organic electroluminescent devices fabricated as described above with M7000 of McScience Inc., and T₉₅ values were measured with the measurement results when the reference luminance was 6,000 cd/m² through the device lifetime measurement system (M6000) manufactured by McScience Inc.

The properties of the organic electroluminescent devices according to the present disclosure are as shown in Table 8 below.

TABLE 8

| | | Driving | Luminous | |
|---|---|---|---|---|
| | | voltage | efficiency | Lifetime |
| | Compound | (V) | (cd/A) | (T₉₅) |
| Example 71 | 003 | 5.34 | 7.07 | 55 |
| Example 72 | 011 | 5.36 | 6.68 | 49 |
| Example 73 | 019 | 5.35 | 6.87 | 52 |
| Example 74 | 046 | 5.39 | 7.06 | 57 |
| Example 75 | 098 | 5.54 | 6.85 | 50 |
| Example 76 | 105 | 5.46 | 6.77 | 62 |
| Example 77 | 159 | 5.38 | 7.11 | 63 |
| Example 78 | 182 | 5.35 | 6.88 | 57 |
| Example 79 | 199 | 5.41 | 6.69 | 54 |
| Example 80 | 224 | 5.37 | 6.46 | 67 |
| Example 81 | 249 | 5.45 | 6.53 | 54 |
| Example 82 | 287 | 5.31 | 6.93 | 51 |
| Example 83 | 319 | 5.47 | 6.61 | 60 |
| Example 84 | 322 | 5.35 | 6.71 | 53 |
| Example 85 | 343 | 5.49 | 6.83 | 56 |
| Example 86 | 367 | 5.54 | 6.74 | 54 |
| Example 87 | 397 | 5.36 | 6.66 | 55 |
| Example 88 | 402 | 5.39 | 6.97 | 53 |
| Example 89 | 431 | 5.38 | 6.81 | 57 |
| Example 90 | 444 | 5.35 | 6.83 | 51 |
| Example 91 | 493 | 5.49 | 6.77 | 54 |
| Example 92 | 562 | 5.45 | 6.80 | 50 |
| Example 93 | 591 | 5.44 | 7.02 | 60 |
| Example 94 | 615 | 5.37 | 6.92 | 62 |
| Comparative | M1 | 6.81 | 5.67 | 35 |
| Example 4 | | | | |
| Comparative | M2 | 5.70 | 6.33 | 43 |
| Example 5 | | | | |

It could be confirmed that the organic light emitting devices of Examples 71 to 94 according to an embodiment of the present disclosure had low driving voltages and excellent efficiencies and lifetimes compared to the organic light emitting devices of Comparative Examples 4 to 5.

### Experimental Example 3

### Experimental Example 3-1. Manufacture of Organic Light

### Emitting Devices

Glass substrates coated with a thin film of indium tin oxide (ITO) to a thickness of 1,500 Å were washed with distilled water and ultrasonic waves. After washing the substrates with distilled water, ultrasonic cleaning was performed on the washed substrates with a solvent such as acetone, methanol, isopropyl alcohol, or the like, and the ultrasonic cleaned substrates were dried, and UVO-treated for 5 minutes using UV in a UV cleaner. Thereafter, after transferring the substrates to a plasma cleaner (PT), plasma treatment was performed in order to increase the ITO work function and remove the residual film in a vacuum state, and then the substrates were transferred to thermal deposition equipment for organic deposition.

Subsequently, after performing air exhaust until the degree of vacuum in the chamber reached 10⁻⁶ torr, an electric current was applied to the cell to evaporate 2-TNATA so that a hole injection layer was deposited to a thickness of 600 Å on the ITO substrates. N,N′-bis(α-naphthyl)-N,N′-diphenyl-4,4′-diamine (NPB) below was put, and an electric current was applied to the cell to evaporate it so that a hole transport layer with a thickness of 300 Å was deposited on the hole injection layer.

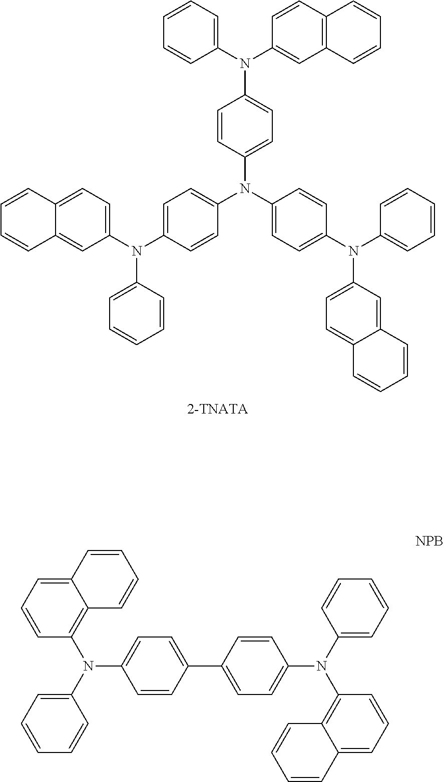 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1]

A light emitting layer was thermal vacuum deposited thereon as follows. The light emitting layer was deposited to a thickness of 500 Å by using the compounds shown in Table 9 below as a host so that an n-Host (n-type host) with good electron transport ability as a single host or a first host and a p-Host (p-type host) with good hole transport ability as a second host were used by a method of depositing two host compounds from one source, and by doping the host with [Ir(piq)₂(acac]] as a red phosphorescent dopant at 3% based on the weight of the host material or doping the host with a green phosphorescent dopant [Ir(ppy)₃] at 7% based on the weight of the host material.

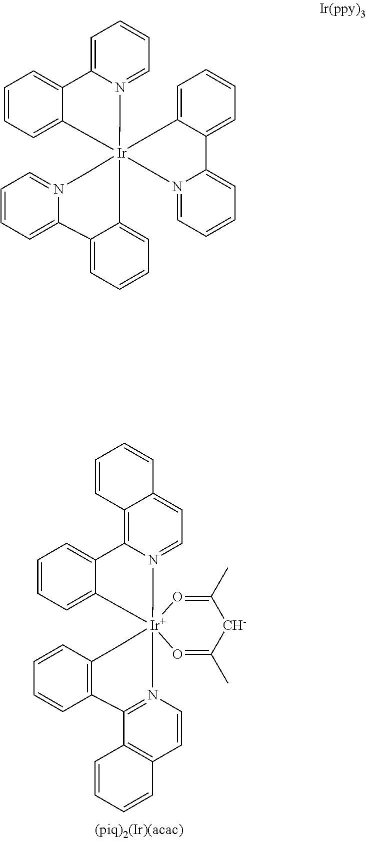

At this time, when two hosts are used, the compounds used as the n-Host are as follows.

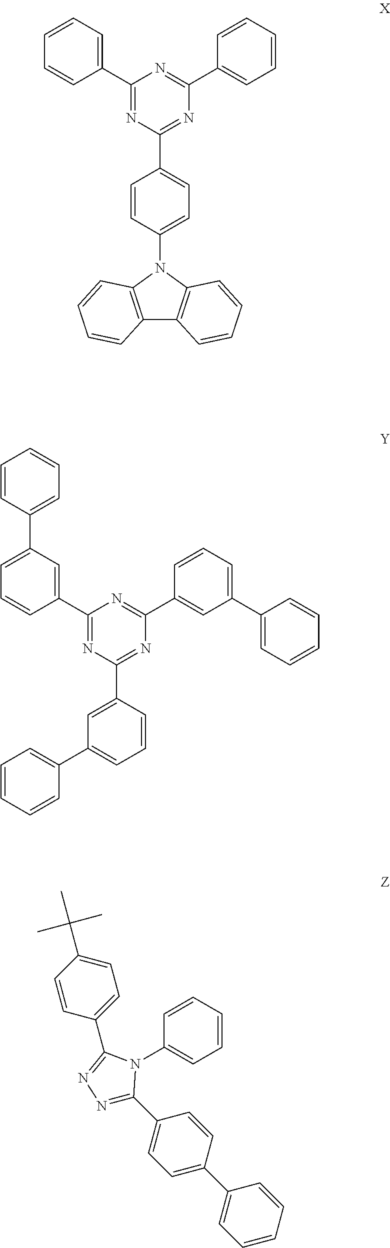 [SMILES: c1ccc(-c2nc(-c3ccccc3)nc(-c3ccc(-n4c5ccccc5c5ccccc54)cc3)n2)cc1 ; c1ccc(-c2cccc(-c3nc(-c4cccc(-c5ccccc5)c4)nc(-c4cccc(-c5ccccc5)c4)n3)c2)cc1 ; CC(C)(C)c1ccc(-c2nnc(-c3ccc(-c4ccccc4)cc3)n2-c2ccccc2)cc1]

Thereafter, BCP was deposited to a thickness of 60 Å as a hole blocking layer, and Alq₃ was deposited thereon to a thickness of 200 Å as an electron transport layer.

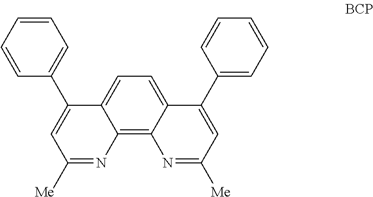 [SMILES: Cc1cc(-c2ccccc2)c2ccc3c(-c4ccccc4)cc(C)nc3c2n1]

Thereafter, after lithium fluoride (LiF) was deposited to a thickness of 10 Å on the electron transport layer to form an electron injection layer, organic electroluminescent devices were manufactured by depositing aluminum (Al) to a thickness of 1,200 Å on the electron injection layer, thereby forming a negative electrode.

Meanwhile, all organic compounds required for fabricating the OLED devices were vacuum sublimated and purified under 10⁻⁶ to 10⁻⁸ torr for each material respectively, and used for OLED fabrication.

At this time, the comparative compounds used as the hosts of Comparative Examples below are as follows.

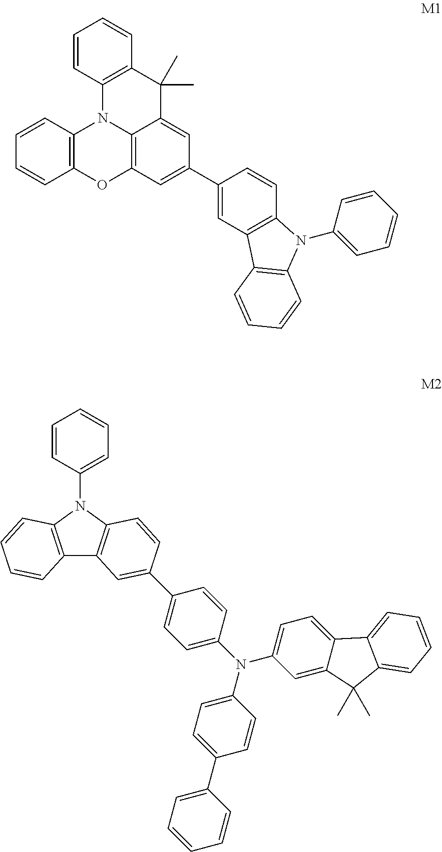 [SMILES: CC1(C)c2ccccc2N2c3ccccc3Oc3cc(-c4ccc5c(c4)c4ccccc4n5-c4ccccc4)cc1c32 ; CC1(C)c2ccccc2-c2ccc(N(c3ccc(-c4ccccc4)cc3)c3ccc(-c4ccc5c(c4)c4ccccc4n5-c4ccccc4)cc3)cc21]

### Experimental Example 3-2. Driving Voltages and Luminous Efficiencies of Organic Light Emitting Devices

Electroluminescence (EL) properties were measured for the organic electroluminescent devices fabricated as described above with M7000 of McScience Inc., and T₉₅ values were measured with the measurement results when the reference luminance was 6,000 cd/m² through the device lifetime measurement system (M6000) manufactured by McScience Inc.

The properties of the organic electroluminescent devices according to the present disclosure are as shown in Table 9 below.

TABLE 9

| | | | Driving | Luminous | Light- | |
|---|---|---|---|---|---|---|
| | First | Second | voltage | efficiency | emitting | Lifetime |
| | host | host | (V) | (cd/A) | color | (T₉₅) |
| Example 95 | 017 | | 4.23 | 23.7 | Red | 62 |
| Example 96 | | | 4.27 | 68.9 | Green | 88 |
| Example 97 | 199 | | 4.21 | 24.1 | Red | 65 |
| Example 98 | | | 4.23 | 73.9 | Green | 83 |
| Example 99 | 224 | | 4.25 | 24.3 | Red | 61 |
| Example 100 | | | 4.29 | 71.7 | Green | 90 |
| Example 101 | 319 | | 4.16 | 23.8 | Red | 58 |
| Example 102 | | | 4.21 | 70.8 | Green | 77 |
| Example 103 | 562 | | 4.09 | 24.6 | Red | 62 |
| Example 104 | | | 4.20 | 72.6 | Green | 81 |
| Example 105 | X | 017 | 3.85 | 42.7 | Red | 136 |
| Example 106 | | 199 | 3.91 | 41.5 | Red | 141 |
| Example 107 | | 224 | 3.97 | 43.6 | Red | 145 |
| Example 108 | | 319 | 3.83 | 42.1 | Red | 132 |
| Example 109 | | 562 | 3.94 | 40.8 | Red | 143 |
| Example 110 | Y | 017 | 3.98 | 115.2 | Green | 154 |
| Example 111 | | 199 | 3.99 | 113.7 | Green | 167 |
| Example 112 | | 224 | 4.05 | 111.6 | Green | 159 |
| Example 113 | | 319 | 3.93 | 104.1 | Green | 163 |
| Example 114 | | 562 | 4.07 | 105.5 | Green | 166 |
| Example 115 | Z | 017 | 3.92 | 39.2 | Red | 132 |
| Example 116 | | 199 | 3.93 | 40.5 | Red | 128 |
| Example 117 | | 224 | 3.75 | 41.2 | Red | 130 |
| Example 118 | | 319 | 4.04 | 40.7 | Red | 135 |
| Example 119 | | 562 | 3.95 | 39.6 | Red | 141 |
| Comparative | M1 | | 4.49 | 21.7 | Red | 50 |
| Example 6 | | | | | | |
| Comparative | X | M1 | 4.25 | 34.5 | Red | 107 |
| Example 7 | | | | | | |
| Comparative | M2 | | 4.54 | 62.9 | Green | 64 |
| Example 8 | | | | | | |
| Comparative | Y | M2 | 4.32 | 89.5 | Green | 134 |
| Example 9 | | | | | | |
| Comparative | Z | M1 | 4.30 | 33.2 | Red | 100 |
| Example 10 | | | | | | |

It could be confirmed from Experimental Example 3 above that the organic light emitting devices of Examples 95 to 104 in which the light emitting layer was formed using the compound according to the present disclosure as a single host material were excellent in luminous efficiencies and lifetime values compared to the organic light emitting devices of Comparative Examples 6 and 8 in which the compound according to the present disclosure was not used as the single host material when forming the light emitting layer using the single host material.

Further, it could be confirmed from Experimental Example 3 above that the organic light emitting devices of Examples 105 to 119 in which the light emitting layer was formed by simultaneously using the first host material corresponding to the n-Host and the compound according to the present disclosure as the second host material corresponding to the p-Host were excellent in luminous efficiencies and lifetime values compared to the organic light emitting devices of Comparative Examples 7, 9, and 10 in which the light emitting layer was formed by simultaneously using the first host material corresponding to the n-Host and a compound other than the compound according to the present disclosure as the second host material corresponding to the p-Host.

Further, it could be confirmed that the luminous efficiencies and lifetime values of the organic light emitting devices of Examples 95 to 104, in which the light emitting layer was formed using the compound according to the present disclosure as the single host material, were similar to or higher than those of the organic light emitting devices of Comparative Examples 6 and 8 in which the light emitting layer was formed by simultaneously using the first host material corresponding to the n-Host and the compound other than the compound according to the present disclosure as the second host material corresponding to the p-Host.

Considering this point, it can be seen that when the compound according to the present disclosure is used as a host material, the luminous efficiency and lifetime of the organic light emitting device can be remarkably improved.

This is so because, when the compound according to the present disclosure is used as the host material, holes and electrons from each charge transfer layer can be efficiently injected into the light emitting layer, and it is judged to be due to the size of the orientation and space formed by the interaction of materials during deposition as described above. That is, as described above, it is judged to be the effect that is caused by the difference in the orientation characteristics and space size of the compound according to the present disclosure and M1 and M2.

The present disclosure is not limited to the above embodiments, but can be manufactured in various different forms, and those of ordinary skill in the art to which the present disclosure pertains will understand that the present disclosure may be implemented in other specific forms without changing the technical spirit or essential features of the present disclosure. Therefore, it should be understood that the embodiments described above are illustrative in all respects and not restrictive.

### EXPLANATION OF REFERENCE NUMERALS

100: Substrate
200: Positive electrode
300: Organic material layer
301: Hole injection layer
302: Hole transport layer
303: Light emitting layer
304: Hole blocking layer
305: Electron transport layer
306: Electron injection layer
400: Negative electrode

1. A heterocyclic compound represented by the following Chemical Formula 1: 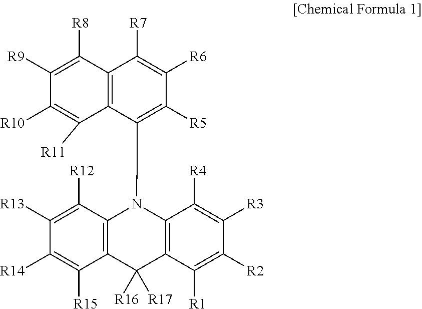
In Chemical Formula 1,
R1 to R17 are the same as or different from each other, each independently selected from the group consisting of: hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102;
—SiR101R102R103; and a group represented by Chemical Formula 2 below, or two or more groups adjacent to each other are bonded to each other to form a substituted or unsubstituted C6 to C60 aromatic hydrocarbon ring or a substituted or unsubstituted C2 to C602 heterocycle, wherein R101, R102, and R103 are the same as or different from each other, and each independently a substituted or unsubstituted C1 to C60 alkyl group; a substituted or unsubstituted C6 to C60 aryl group; or a substituted or unsubstituted C2 to C60 heteroaryl group,
at least one of R1 to R4 and R12 to R15 is a substituted or unsubstituted C6 to C60 aryl group; a substituted or unsubstituted C2 to C60 heteroaryl group; or a group represented by Chemical Formula 2 below,
at least one of R5 to R11 is a substituted or unsubstituted C6 to C60 aryl group; a substituted or unsubstituted C2 to C60 heteroaryl group; or a group represented by Chemical Formula 2 below, 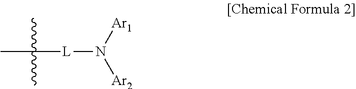
In Chemical Formula 2,
Ar1 and Ar2 are the same as or different from each other, and each independently a substituted or unsubstituted C6 to C60 aryl group; or a substituted or unsubstituted C2 to C60 heteroaryl group, and
L is a direct bond; a substituted or unsubstituted C6 to C60 arylene group; or a substituted or unsubstituted C2 to C60 heteroarylene group.

2. The heterocyclic compound of claim 1, wherein R16 and R17 are the same as or different from each other, and each independently a substituted or unsubstituted C1 to C20 alkyl group.

3. The heterocyclic compound of claim 2, wherein R5 in the Chemical Formula 1 is a substituted or unsubstituted C6 to C60 aryl group; a substituted or unsubstituted C2 to C60 heteroaryl group; or the group represented by Chemical Formula 2, and
at least one of R1 to R3 and R13 to R15 is a substituted or unsubstituted C6 to C60 aryl group; a substituted or unsubstituted C2 to C60 heteroaryl group; or the group represented by Chemical Formula 2.

4. The heterocyclic compound of claim 3, wherein when R5 in the Chemical Formula 1 is a substituted or unsubstituted C6 to C60 aryl group; or a substituted or unsubstituted C2 to C60 heteroaryl group, at least one of R1 to R3 and R13 to R15 is the group represented by Chemical Formula 2, and
when R5 is the group represented by Chemical Formula 2, at least one of R1 to R3 and R13 to R15 is a substituted or unsubstituted C6 to C60 aryl group; or a substituted or unsubstituted C2 to C60 heteroaryl group.

5. The heterocyclic compound of claim 1, wherein the content of deuterium is 30% to 100% based on the total number of hydrogen atoms and deuterium atoms in the Chemical Formula 1.

6. The heterocyclic compound of claim 1, wherein the Chemical Formula 1 is represented by any one of the following compounds:

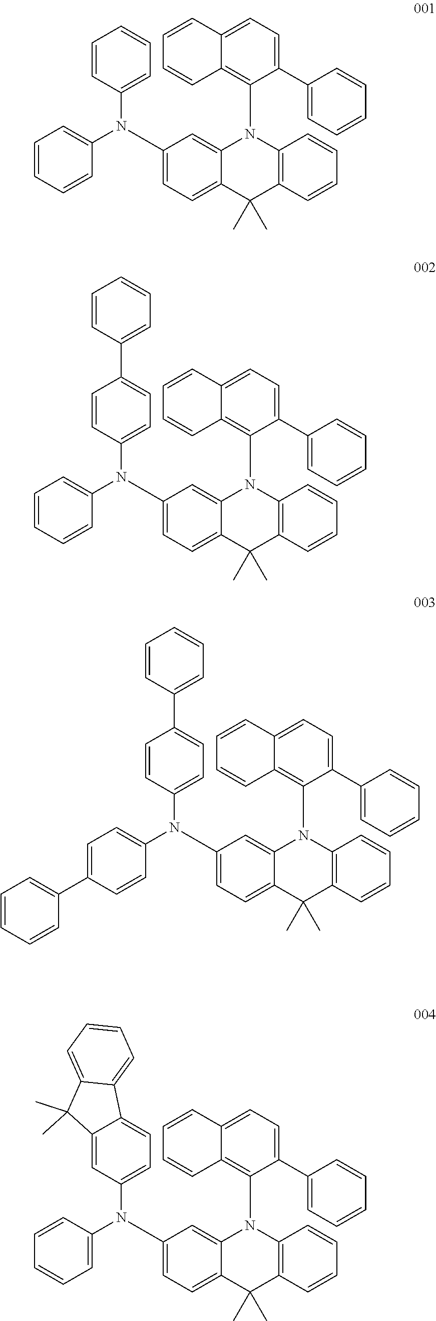 [SMILES: CC1(C)c2ccccc2N(c2c(-c3ccccc3)ccc3ccccc23)c2cc(N(c3ccccc3)c3ccccc3)ccc21 ; CC1(C)c2ccccc2N(c2c(-c3ccccc3)ccc3ccccc23)c2cc(N(c3ccccc3)c3ccc(-c4ccccc4)cc3)ccc21 ; CC1(C)c2ccccc2N(c2c(-c3ccccc3)ccc3ccccc23)c2cc(N(c3ccc(-c4ccccc4)cc3)c3ccc(-c4ccccc4)cc3)ccc21 ; CC1(C)c2ccccc2-c2ccc(N(c3ccccc3)c3ccc4c(c3)N(c3c(-c5ccccc5)ccc5ccccc35)c3ccccc3C4(C)C)cc21]

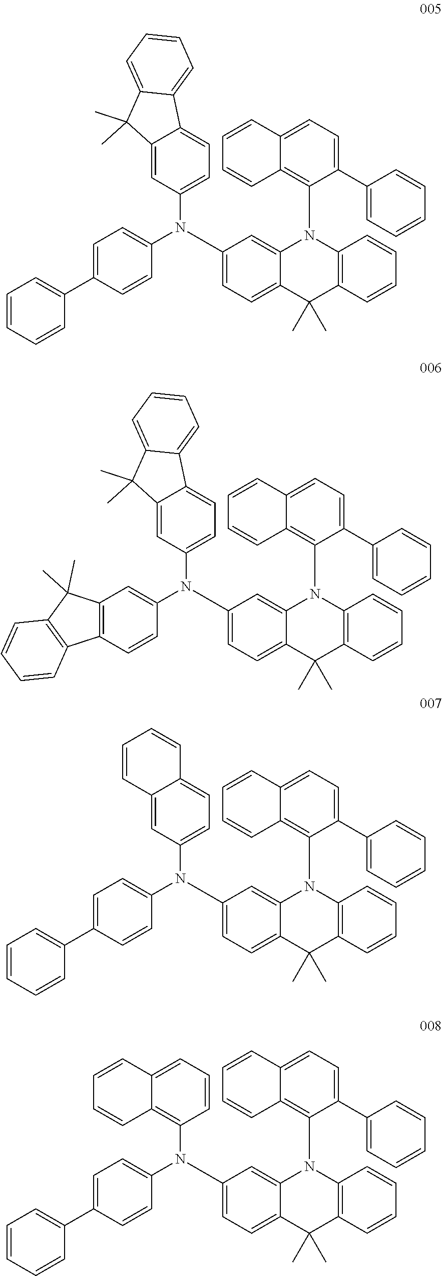 [SMILES: CC1(C)c2ccccc2-c2ccc(N(c3ccc(-c4ccccc4)cc3)c3ccc4c(c3)N(c3c(-c5ccccc5)ccc5ccccc35)c3ccccc3C4(C)C)cc21 ; CC1(C)c2ccccc2-c2ccc(N(c3ccc4c(c3)N(c3c(-c5ccccc5)ccc5ccccc35)c3ccccc3C4(C)C)c3ccc4c(c3)C(C)(C)c3ccccc3-4)cc21 ; CC1(C)c2ccccc2N(c2c(-c3ccccc3)ccc3ccccc23)c2cc(N(c3ccc(-c4ccccc4)cc3)c3ccc4ccccc4c3)ccc21 ; CC1(C)c2ccccc2N(c2c(-c3ccccc3)ccc3ccccc23)c2cc(N(c3ccc(-c4ccccc4)cc3)c3cccc4ccccc34)ccc21]

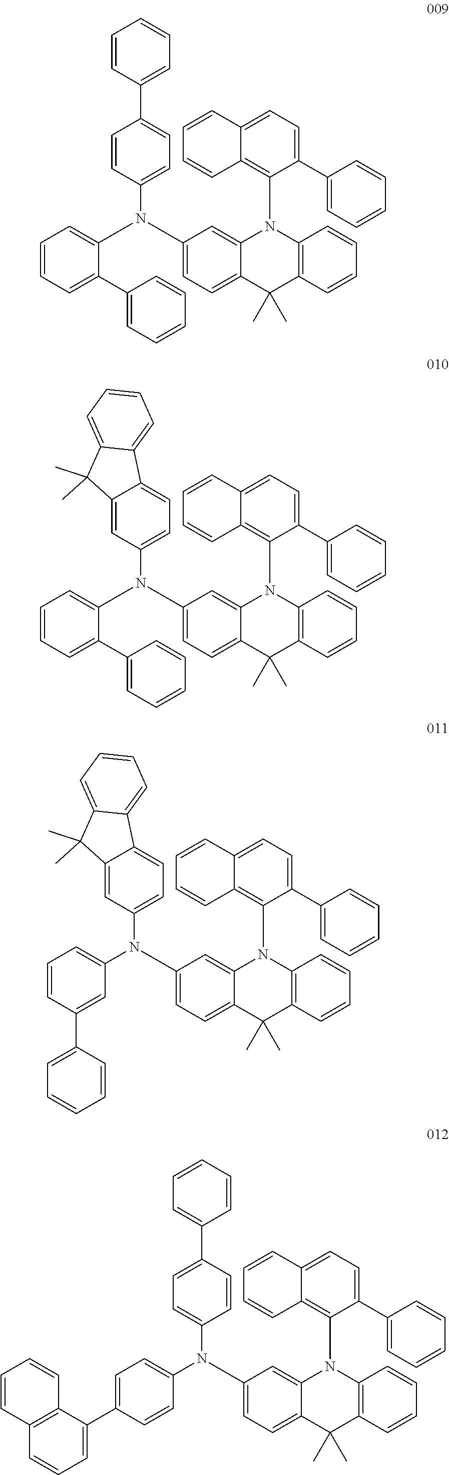 [SMILES: CC1(C)c2ccccc2N(c2c(-c3ccccc3)ccc3ccccc23)c2cc(N(c3ccc(-c4ccccc4)cc3)c3ccccc3-c3ccccc3)ccc21 ; CC1(C)c2ccccc2-c2ccc(N(c3ccc4c(c3)N(c3c(-c5ccccc5)ccc5ccccc35)c3ccccc3C4(C)C)c3ccccc3-c3ccccc3)cc21 ; CC1(C)c2ccccc2-c2ccc(N(c3cccc(-c4ccccc4)c3)c3ccc4c(c3)N(c3c(-c5ccccc5)ccc5ccccc35)c3ccccc3C4(C)C)cc21 ; CC1(C)c2ccccc2N(c2c(-c3ccccc3)ccc3ccccc23)c2cc(N(c3ccc(-c4ccccc4)cc3)c3ccc(-c4cccc5ccccc45)cc3)ccc21]

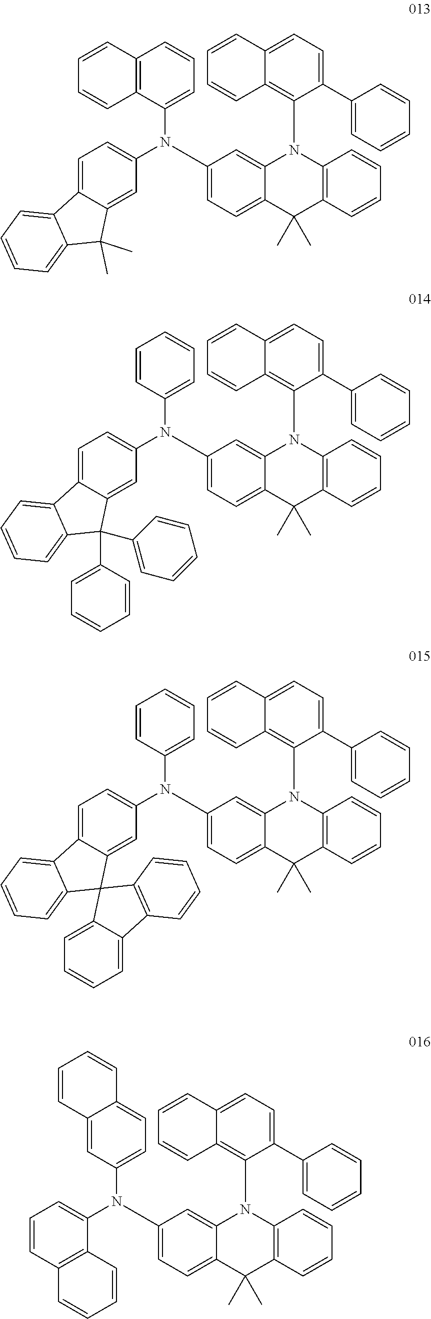 [SMILES: CC1(C)c2ccccc2-c2ccc(N(c3ccc4c(c3)N(c3c(-c5ccccc5)ccc5ccccc35)c3ccccc3C4(C)C)c3cccc4ccccc34)cc21 ; CC1(C)c2ccccc2N(c2c(-c3ccccc3)ccc3ccccc23)c2cc(N(c3ccccc3)c3ccc4c(c3)C(c3ccccc3)(c3ccccc3)c3ccccc3-4)ccc21 ; CC1(C)c2ccccc2N(c2c(-c3ccccc3)ccc3ccccc23)c2cc(N(c3ccccc3)c3ccc4c(c3)C3(c5ccccc5-c5ccccc53)c3ccccc3-4)ccc21 ; CC1(C)c2ccccc2N(c2c(-c3ccccc3)ccc3ccccc23)c2cc(N(c3ccc4ccccc4c3)c3cccc4ccccc34)ccc21]

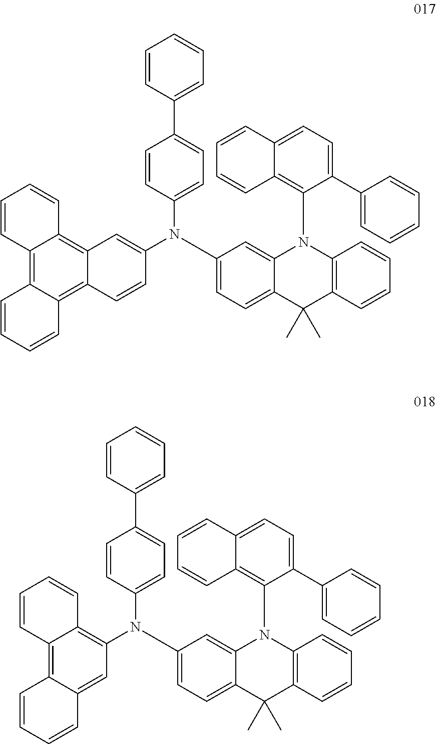 [SMILES: CC1(C)c2ccccc2N(c2c(-c3ccccc3)ccc3ccccc23)c2cc(N(c3ccc(-c4ccccc4)cc3)c3ccc4c5ccccc5c5ccccc5c4c3)ccc21 ; CC1(C)c2ccccc2N(c2c(-c3ccccc3)ccc3ccccc23)c2cc(N(c3ccc(-c4ccccc4)cc3)c3cc4ccccc4c4ccccc34)ccc21]

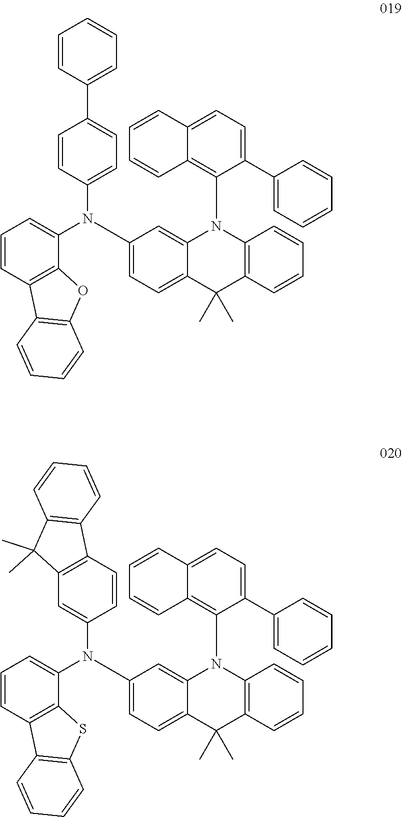 [SMILES: CC1(C)c2ccccc2N(c2c(-c3ccccc3)ccc3ccccc23)c2cc(N(c3ccc(-c4ccccc4)cc3)c3cccc4c3oc3ccccc34)ccc21 ; CC1(C)c2ccccc2-c2ccc(N(c3ccc4c(c3)N(c3c(-c5ccccc5)ccc5ccccc35)c3ccccc3C4(C)C)c3cccc4c3sc3ccccc34)cc21]

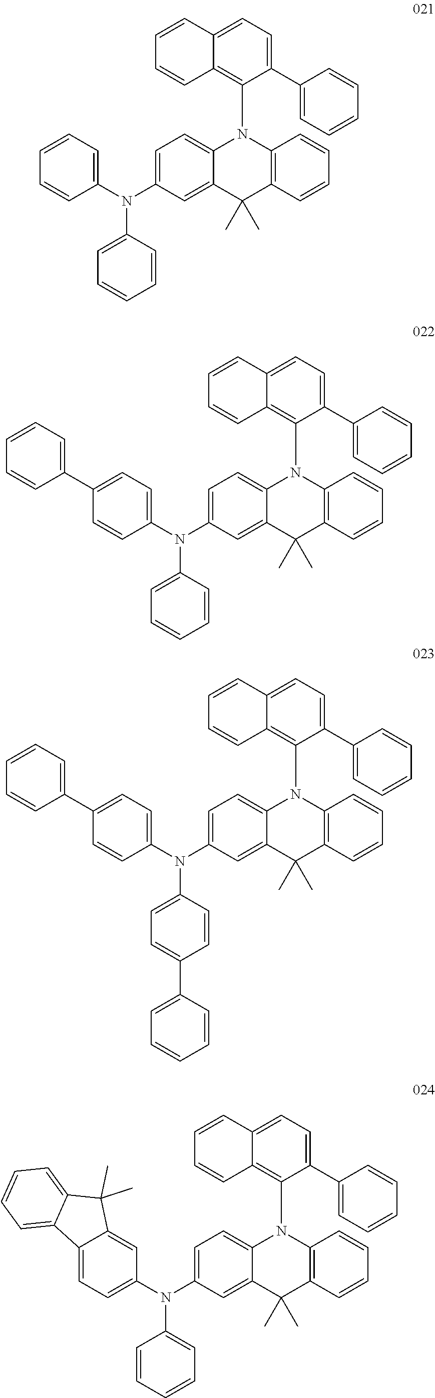 [SMILES: CC1(C)c2ccccc2N(c2c(-c3ccccc3)ccc3ccccc23)c2ccc(N(c3ccccc3)c3ccccc3)cc21 ; CC1(C)c2ccccc2N(c2c(-c3ccccc3)ccc3ccccc23)c2ccc(N(c3ccccc3)c3ccc(-c4ccccc4)cc3)cc21 ; CC1(C)c2ccccc2N(c2c(-c3ccccc3)ccc3ccccc23)c2ccc(N(c3ccc(-c4ccccc4)cc3)c3ccc(-c4ccccc4)cc3)cc21 ; CC1(C)c2ccccc2-c2ccc(N(c3ccccc3)c3ccc4c(c3)C(C)(C)c3ccccc3N4c3c(-c4ccccc4)ccc4ccccc34)cc21]

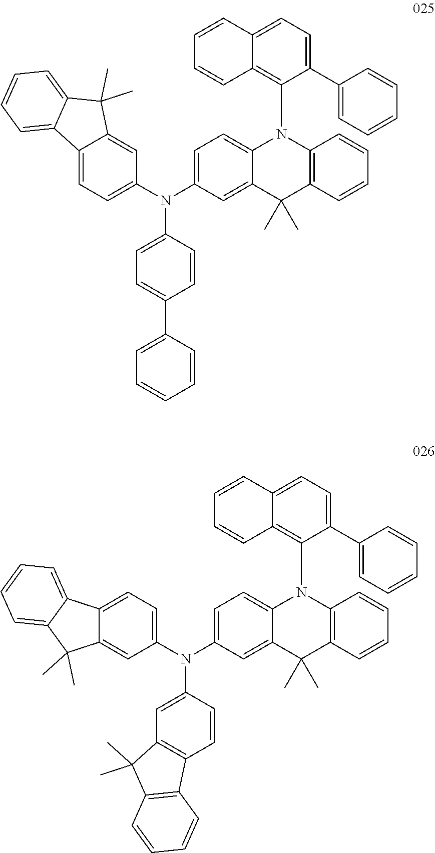 [SMILES: CC1(C)c2ccccc2-c2ccc(N(c3ccc(-c4ccccc4)cc3)c3ccc4c(c3)C(C)(C)c3ccccc3N4c3c(-c4ccccc4)ccc4ccccc34)cc21 ; CC1(C)c2ccccc2-c2ccc(N(c3ccc4c(c3)C(C)(C)c3ccccc3-4)c3ccc4c(c3)C(C)(C)c3ccccc3N4c3c(-c4ccccc4)ccc4ccccc34)cc21]

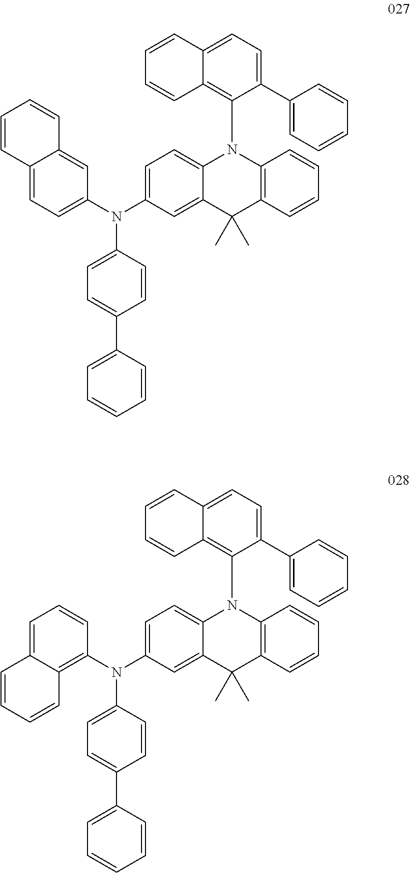 [SMILES: CC1(C)c2ccccc2N(c2c(-c3ccccc3)ccc3ccccc23)c2ccc(N(c3ccc(-c4ccccc4)cc3)c3ccc4ccccc4c3)cc21 ; CC1(C)c2ccccc2N(c2c(-c3ccccc3)ccc3ccccc23)c2ccc(N(c3ccc(-c4ccccc4)cc3)c3cccc4ccccc34)cc21]

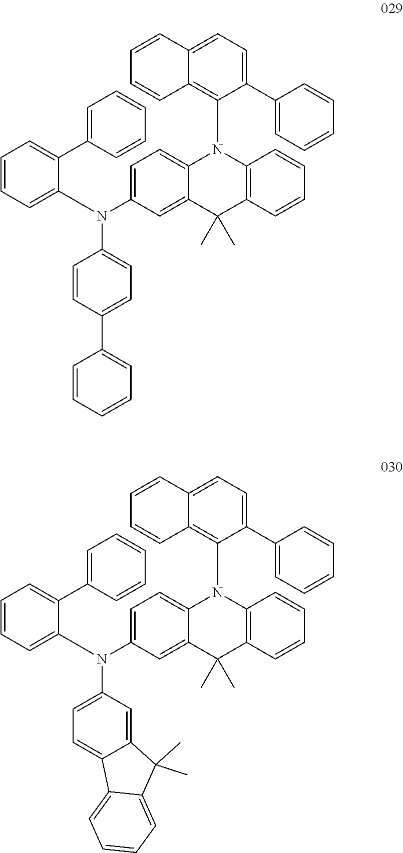 [SMILES: CC1(C)c2ccccc2N(c2c(-c3ccccc3)ccc3ccccc23)c2ccc(N(c3ccc(-c4ccccc4)cc3)c3ccccc3-c3ccccc3)cc21 ; CC1(C)c2ccccc2-c2ccc(N(c3ccc4c(c3)C(C)(C)c3ccccc3N4c3c(-c4ccccc4)ccc4ccccc34)c3ccccc3-c3ccccc3)cc21]

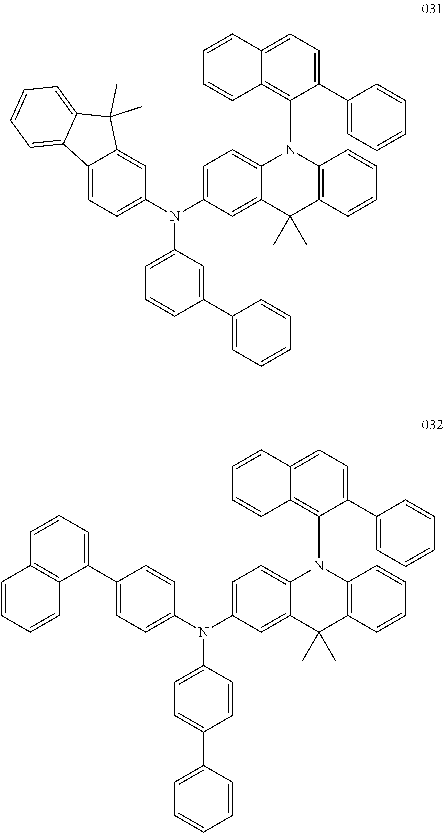 [SMILES: CC1(C)c2ccccc2-c2ccc(N(c3cccc(-c4ccccc4)c3)c3ccc4c(c3)C(C)(C)c3ccccc3N4c3c(-c4ccccc4)ccc4ccccc34)cc21 ; CC1(C)c2ccccc2N(c2c(-c3ccccc3)ccc3ccccc23)c2ccc(N(c3ccc(-c4ccccc4)cc3)c3ccc(-c4cccc5ccccc45)cc3)cc21]

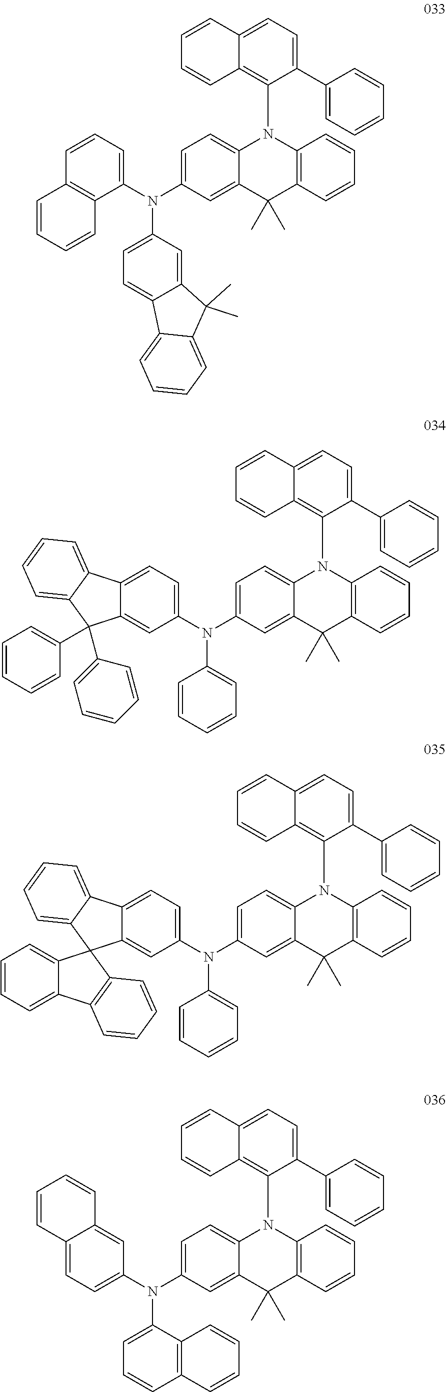 [SMILES: CC1(C)c2ccccc2-c2ccc(N(c3ccc4c(c3)C(C)(C)c3ccccc3N4c3c(-c4ccccc4)ccc4ccccc34)c3cccc4ccccc34)cc21 ; CC1(C)c2ccccc2N(c2c(-c3ccccc3)ccc3ccccc23)c2ccc(N(c3ccccc3)c3ccc4c(c3)C(c3ccccc3)(c3ccccc3)c3ccccc3-4)cc21 ; CC1(C)c2ccccc2N(c2c(-c3ccccc3)ccc3ccccc23)c2ccc(N(c3ccccc3)c3ccc4c(c3)C3(c5ccccc5-c5ccccc53)c3ccccc3-4)cc21 ; CC1(C)c2ccccc2N(c2c(-c3ccccc3)ccc3ccccc23)c2ccc(N(c3ccc4ccccc4c3)c3cccc4ccccc34)cc21]

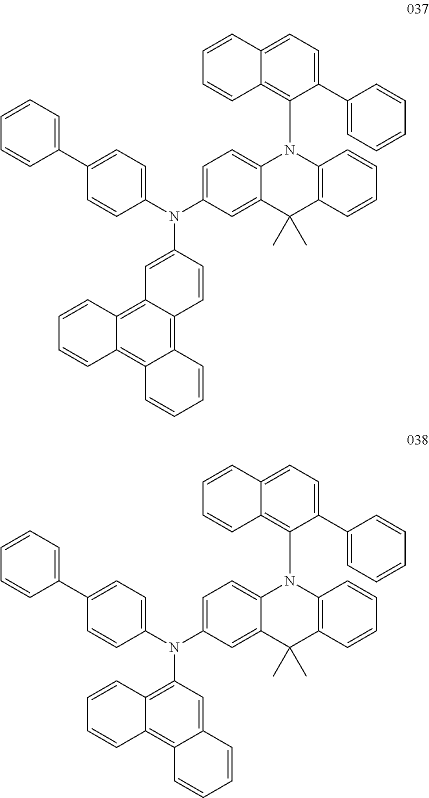 [SMILES: CC1(C)c2ccccc2N(c2c(-c3ccccc3)ccc3ccccc23)c2ccc(N(c3ccc(-c4ccccc4)cc3)c3ccc4c5ccccc5c5ccccc5c4c3)cc21 ; CC1(C)c2ccccc2N(c2c(-c3ccccc3)ccc3ccccc23)c2ccc(N(c3ccc(-c4ccccc4)cc3)c3cc4ccccc4c4ccccc34)cc21]

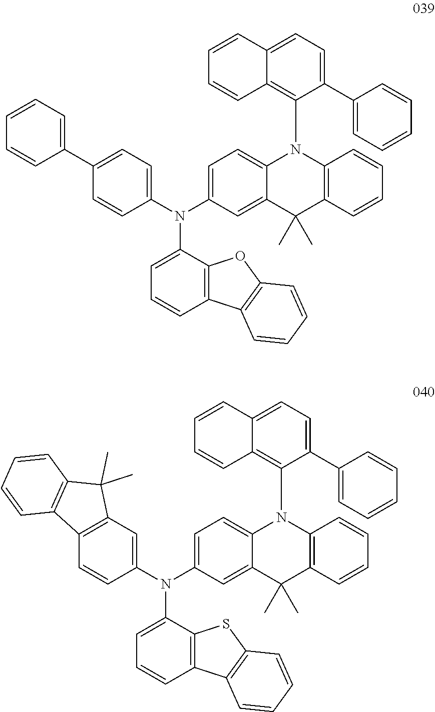 [SMILES: CC1(C)c2ccccc2N(c2c(-c3ccccc3)ccc3ccccc23)c2ccc(N(c3ccc(-c4ccccc4)cc3)c3cccc4c3oc3ccccc34)cc21 ; CC1(C)c2ccccc2-c2ccc(N(c3ccc4c(c3)C(C)(C)c3ccccc3N4c3c(-c4ccccc4)ccc4ccccc34)c3cccc4c3sc3ccccc34)cc21]

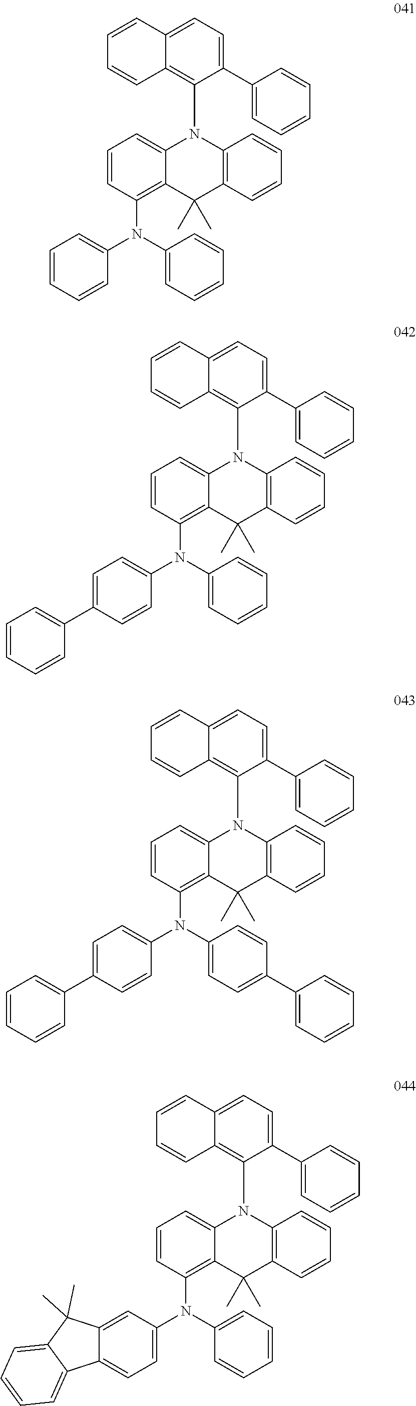

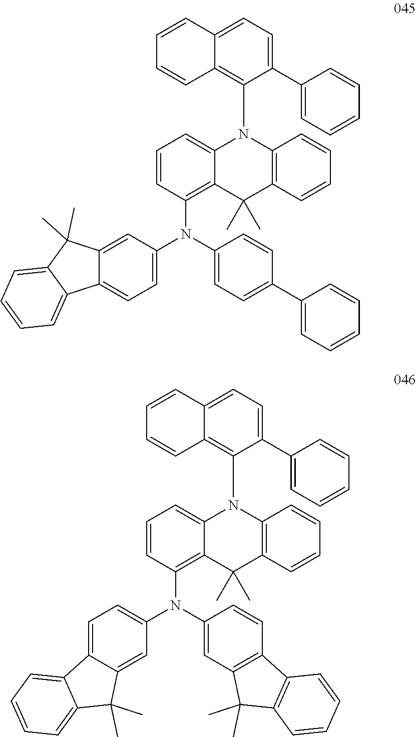 [SMILES: CC1(C)c2ccccc2-c2ccc(N(c3ccc(-c4ccccc4)cc3)c3cccc4c3C(C)(C)c3ccccc3N4c3c(-c4ccccc4)ccc4ccccc34)cc21 ; CC1(C)c2ccccc2-c2ccc(N(c3ccc4c(c3)C(C)(C)c3ccccc3-4)c3cccc4c3C(C)(C)c3ccccc3N4c3c(-c4ccccc4)ccc4ccccc34)cc21]

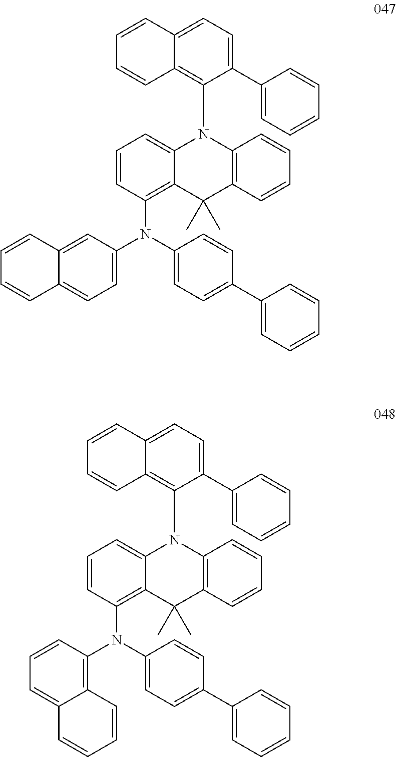 [SMILES: CC1(C)c2ccccc2N(c2c(-c3ccccc3)ccc3ccccc23)c2cccc(N(c3ccc(-c4ccccc4)cc3)c3ccc4ccccc4c3)c21 ; CC1(C)c2ccccc2N(c2c(-c3ccccc3)ccc3ccccc23)c2cccc(N(c3ccc(-c4ccccc4)cc3)c3cccc4ccccc34)c21]

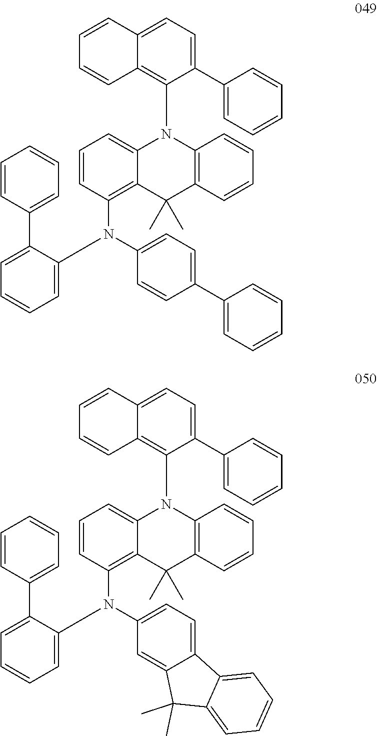 [SMILES: CC1(C)c2ccccc2N(c2c(-c3ccccc3)ccc3ccccc23)c2cccc(N(c3ccc(-c4ccccc4)cc3)c3ccccc3-c3ccccc3)c21 ; CC1(C)c2ccccc2-c2ccc(N(c3ccccc3-c3ccccc3)c3cccc4c3C(C)(C)c3ccccc3N4c3c(-c4ccccc4)ccc4ccccc34)cc21]

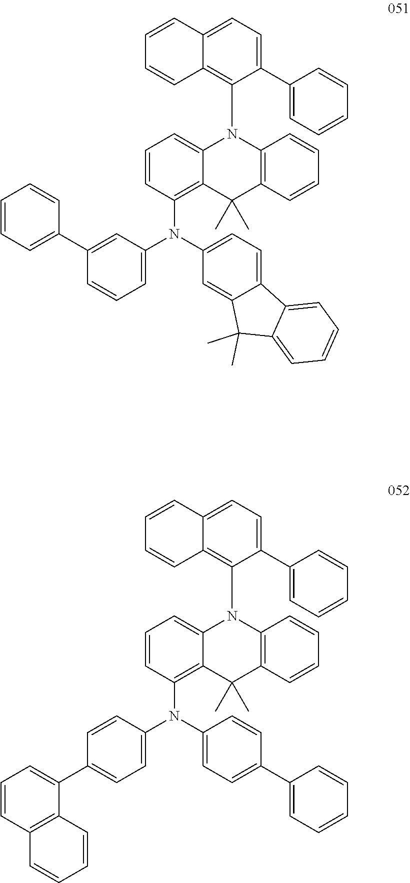 [SMILES: CC1(C)c2ccccc2-c2ccc(N(c3cccc(-c4ccccc4)c3)c3cccc4c3C(C)(C)c3ccccc3N4c3c(-c4ccccc4)ccc4ccccc34)cc21 ; CC1(C)c2ccccc2N(c2c(-c3ccccc3)ccc3ccccc23)c2cccc(N(c3ccc(-c4ccccc4)cc3)c3ccc(-c4cccc5ccccc45)cc3)c21]

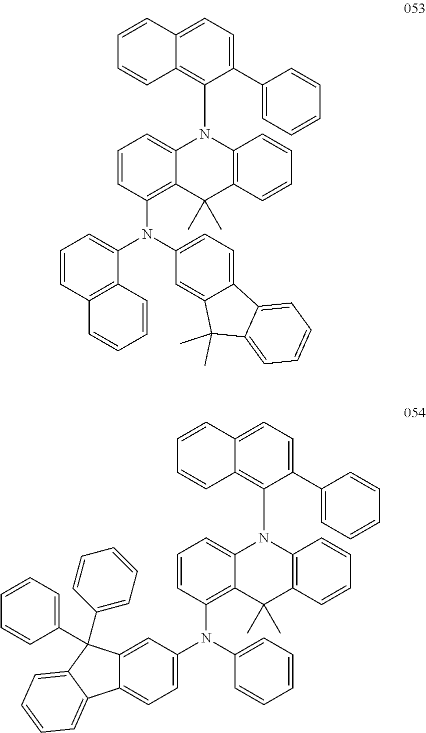 [SMILES: CC1(C)c2ccccc2-c2ccc(N(c3cccc4c3C(C)(C)c3ccccc3N4c3c(-c4ccccc4)ccc4ccccc34)c3cccc4ccccc34)cc21 ; CC1(C)c2ccccc2N(c2c(-c3ccccc3)ccc3ccccc23)c2cccc(N(c3ccccc3)c3ccc4c(c3)C(c3ccccc3)(c3ccccc3)c3ccccc3-4)c21]

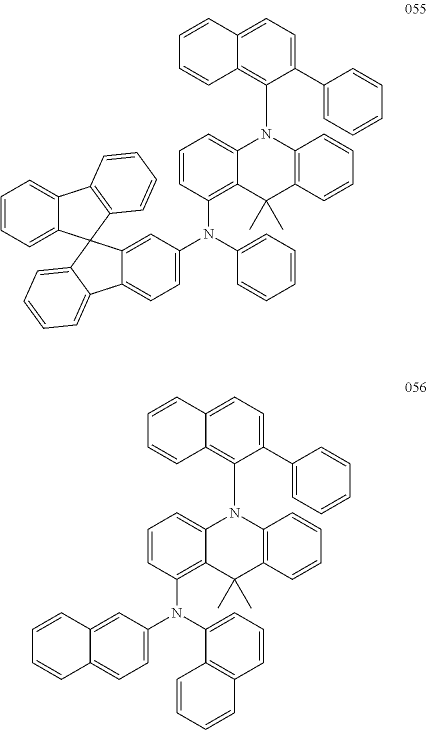 [SMILES: CC1(C)c2ccccc2N(c2c(-c3ccccc3)ccc3ccccc23)c2cccc(N(c3ccccc3)c3ccc4c(c3)C3(c5ccccc5-c5ccccc53)c3ccccc3-4)c21 ; CC1(C)c2ccccc2N(c2c(-c3ccccc3)ccc3ccccc23)c2cccc(N(c3ccc4ccccc4c3)c3cccc4ccccc34)c21]

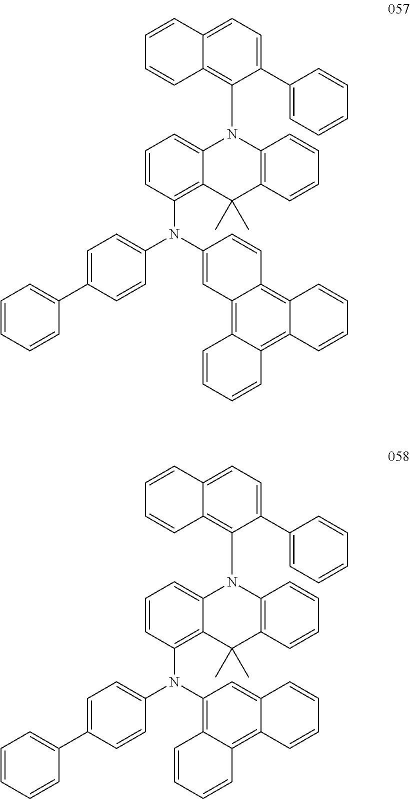 [SMILES: CC1(C)c2ccccc2N(c2c(-c3ccccc3)ccc3ccccc23)c2cccc(N(c3ccc(-c4ccccc4)cc3)c3ccc4c5ccccc5c5ccccc5c4c3)c21 ; CC1(C)c2ccccc2N(c2c(-c3ccccc3)ccc3ccccc23)c2cccc(N(c3ccc(-c4ccccc4)cc3)c3cc4ccccc4c4ccccc34)c21]

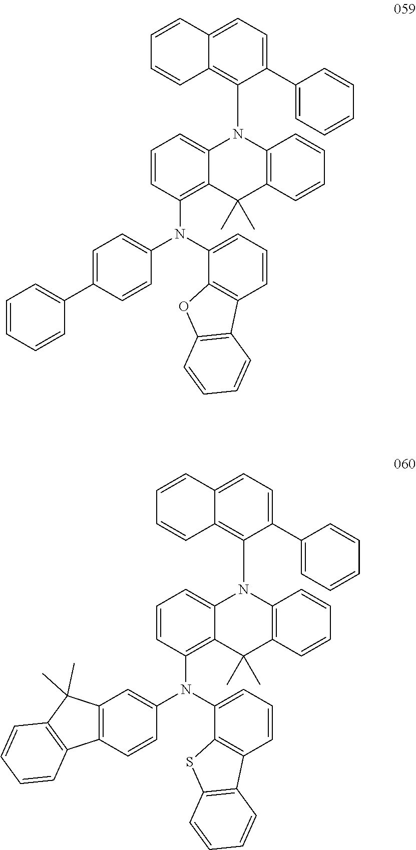 [SMILES: CC1(C)c2ccccc2N(c2c(-c3ccccc3)ccc3ccccc23)c2cccc(N(c3ccc(-c4ccccc4)cc3)c3cccc4c3oc3ccccc34)c21 ; CC1(C)c2ccccc2-c2ccc(N(c3cccc4c3C(C)(C)c3ccccc3N4c3c(-c4ccccc4)ccc4ccccc34)c3cccc4c3sc3ccccc34)cc21]

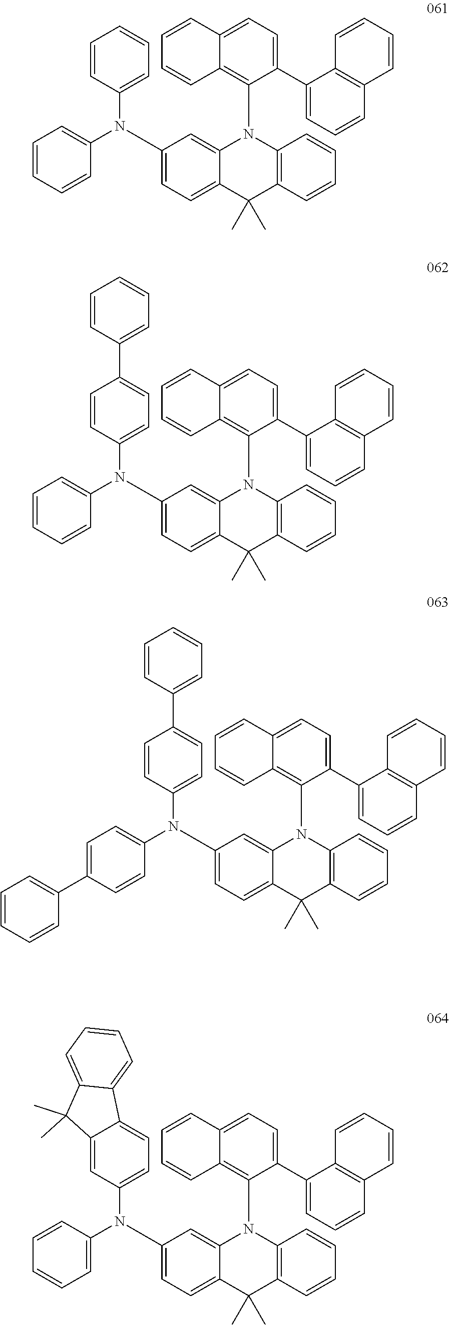 [SMILES: CC1(C)c2ccccc2N(c2c(-c3cccc4ccccc34)ccc3ccccc23)c2cc(N(c3ccccc3)c3ccccc3)ccc21 ; CC1(C)c2ccccc2N(c2c(-c3cccc4ccccc34)ccc3ccccc23)c2cc(N(c3ccccc3)c3ccc(-c4ccccc4)cc3)ccc21 ; CC1(C)c2ccccc2N(c2c(-c3cccc4ccccc34)ccc3ccccc23)c2cc(N(c3ccc(-c4ccccc4)cc3)c3ccc(-c4ccccc4)cc3)ccc21 ; CC1(C)c2ccccc2-c2ccc(N(c3ccccc3)c3ccc4c(c3)N(c3c(-c5cccc6ccccc56)ccc5ccccc35)c3ccccc3C4(C)C)cc21]

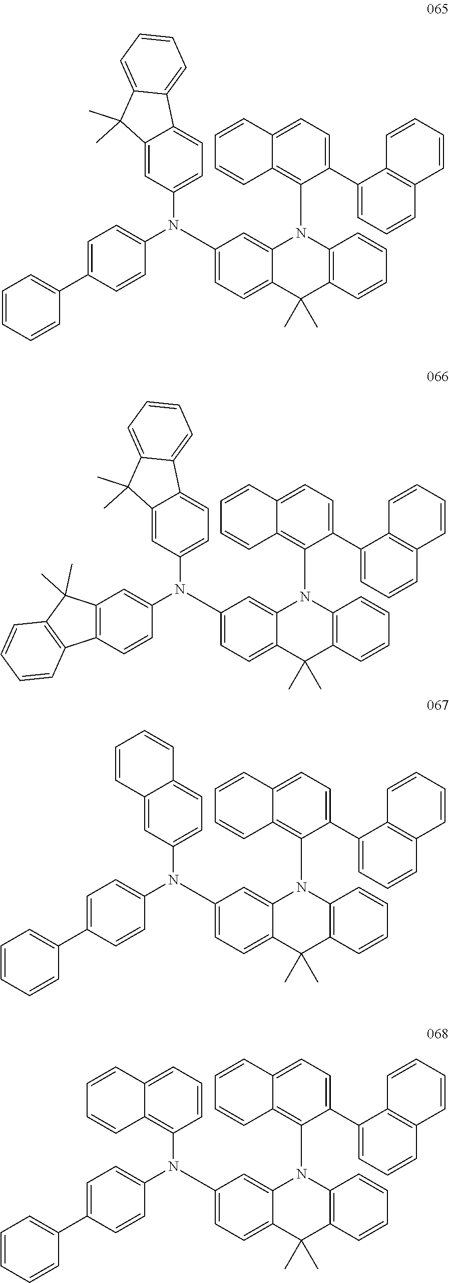 [SMILES: CC1(C)c2ccccc2-c2ccc(N(c3ccc(-c4ccccc4)cc3)c3ccc4c(c3)N(c3c(-c5cccc6ccccc56)ccc5ccccc35)c3ccccc3C4(C)C)cc21 ; CC1(C)c2ccccc2-c2ccc(N(c3ccc4c(c3)N(c3c(-c5cccc6ccccc56)ccc5ccccc35)c3ccccc3C4(C)C)c3ccc4c(c3)C(C)(C)c3ccccc3-4)cc21 ; CC1(C)c2ccccc2N(c2c(-c3cccc4ccccc34)ccc3ccccc23)c2cc(N(c3ccc(-c4ccccc4)cc3)c3ccc4ccccc4c3)ccc21 ; CC1(C)c2ccccc2N(c2c(-c3cccc4ccccc34)ccc3ccccc23)c2cc(N(c3ccc(-c4ccccc4)cc3)c3cccc4ccccc34)ccc21]

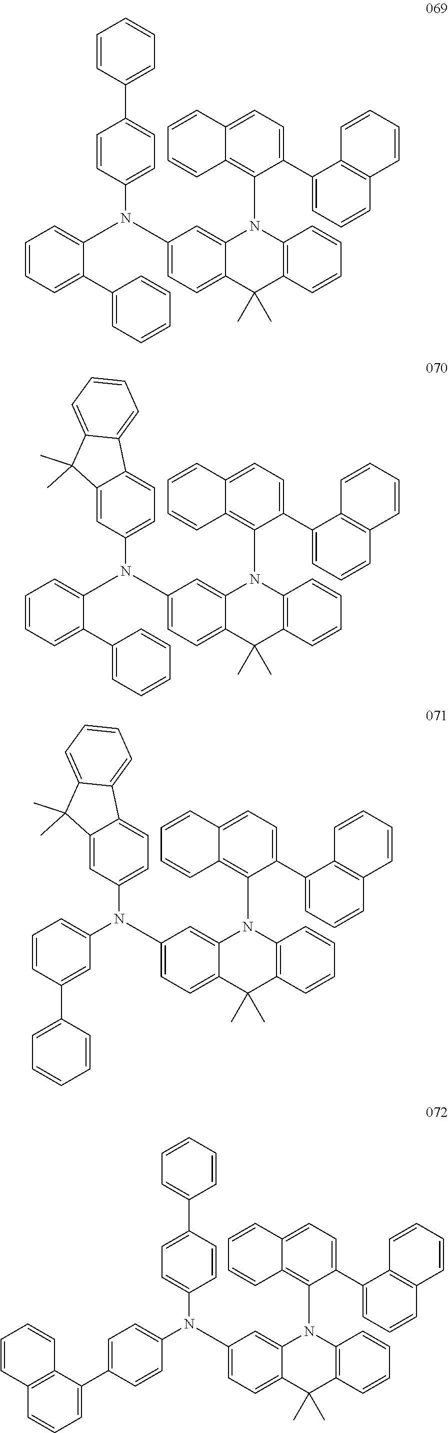

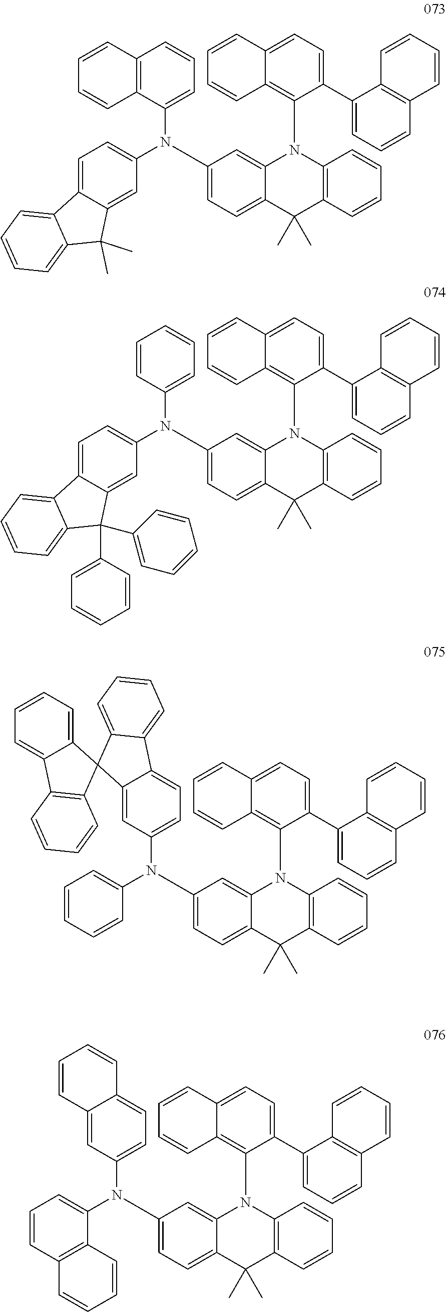 [SMILES: CC1(C)c2ccccc2-c2ccc(N(c3ccc4c(c3)N(c3c(-c5cccc6ccccc56)ccc5ccccc35)c3ccccc3C4(C)C)c3cccc4ccccc34)cc21 ; CC1(C)c2ccccc2N(c2c(-c3cccc4ccccc34)ccc3ccccc23)c2cc(N(c3ccccc3)c3ccc4c(c3)C(c3ccccc3)(c3ccccc3)c3ccccc3-4)ccc21 ; CC1(C)c2ccccc2N(c2c(-c3cccc4ccccc34)ccc3ccccc23)c2cc(N(c3ccccc3)c3ccc4c(c3)C3(c5ccccc5-c5ccccc53)c3ccccc3-4)ccc21 ; CC1(C)c2ccccc2N(c2c(-c3cccc4ccccc34)ccc3ccccc23)c2cc(N(c3ccc4ccccc4c3)c3cccc4ccccc34)ccc21]

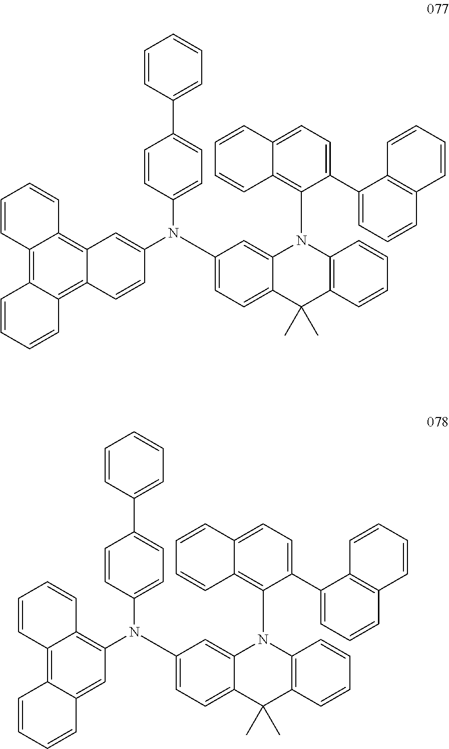 [SMILES: CC1(C)c2ccccc2N(c2c(-c3cccc4ccccc34)ccc3ccccc23)c2cc(N(c3ccc(-c4ccccc4)cc3)c3ccc4c5ccccc5c5ccccc5c4c3)ccc21 ; CC1(C)c2ccccc2N(c2c(-c3cccc4ccccc34)ccc3ccccc23)c2cc(N(c3ccc(-c4ccccc4)cc3)c3cc4ccccc4c4ccccc34)ccc21]

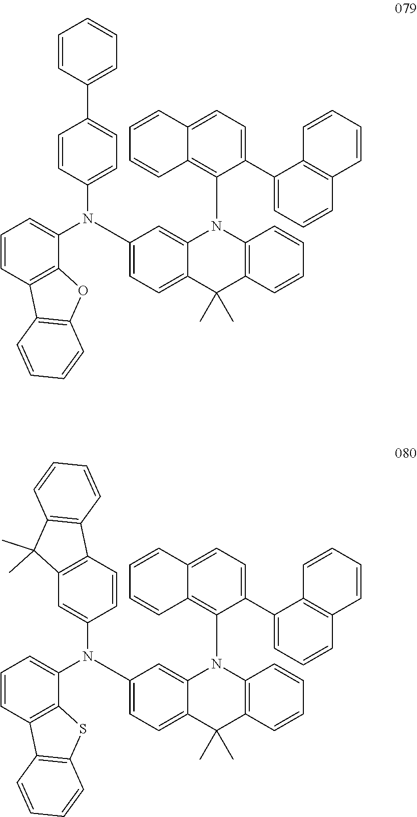 [SMILES: CC1(C)c2ccccc2N(c2c(-c3cccc4ccccc34)ccc3ccccc23)c2cc(N(c3ccc(-c4ccccc4)cc3)c3cccc4c3oc3ccccc34)ccc21 ; CC1(C)c2ccccc2-c2ccc(N(c3ccc4c(c3)N(c3c(-c5cccc6ccccc56)ccc5ccccc35)c3ccccc3C4(C)C)c3cccc4c3sc3ccccc34)cc21]

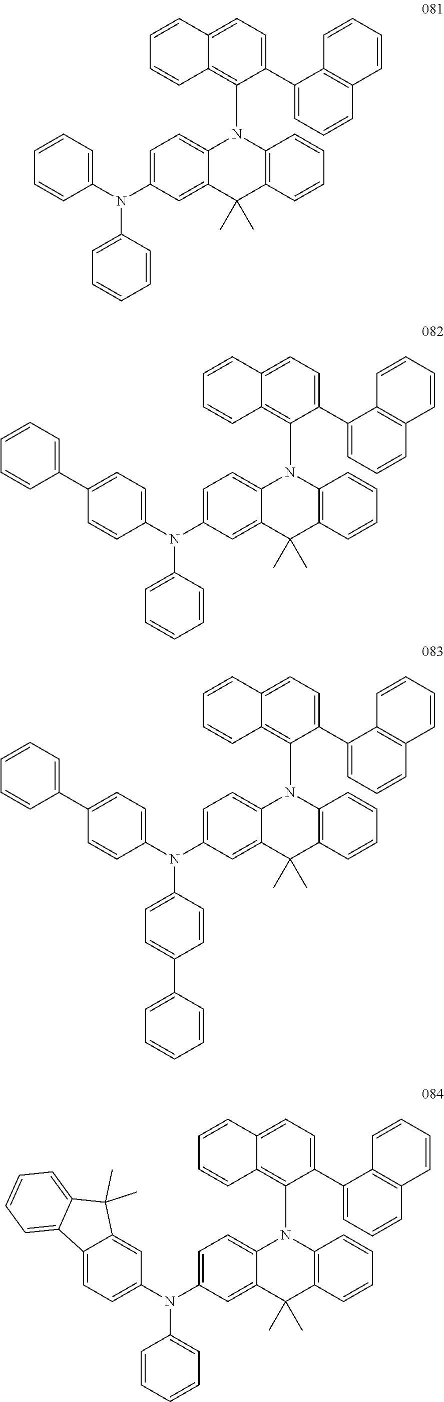 [SMILES: CC1(C)c2ccccc2N(c2c(-c3cccc4ccccc34)ccc3ccccc23)c2ccc(N(c3ccccc3)c3ccccc3)cc21 ; CC1(C)c2ccccc2N(c2c(-c3cccc4ccccc34)ccc3ccccc23)c2ccc(N(c3ccccc3)c3ccc(-c4ccccc4)cc3)cc21 ; CC1(C)c2ccccc2N(c2c(-c3cccc4ccccc34)ccc3ccccc23)c2ccc(N(c3ccc(-c4ccccc4)cc3)c3ccc(-c4ccccc4)cc3)cc21 ; CC1(C)c2ccccc2-c2ccc(N(c3ccccc3)c3ccc4c(c3)C(C)(C)c3ccccc3N4c3c(-c4cccc5ccccc45)ccc4ccccc34)cc21]

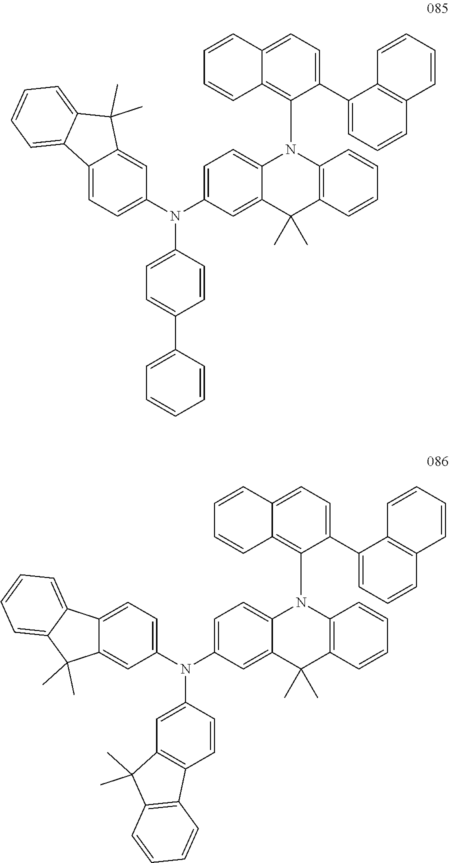 [SMILES: CC1(C)c2ccccc2-c2ccc(N(c3ccc(-c4ccccc4)cc3)c3ccc4c(c3)C(C)(C)c3ccccc3N4c3c(-c4cccc5ccccc45)ccc4ccccc34)cc21 ; CC1(C)c2ccccc2-c2ccc(N(c3ccc4c(c3)C(C)(C)c3ccccc3-4)c3ccc4c(c3)C(C)(C)c3ccccc3N4c3c(-c4cccc5ccccc45)ccc4ccccc34)cc21]

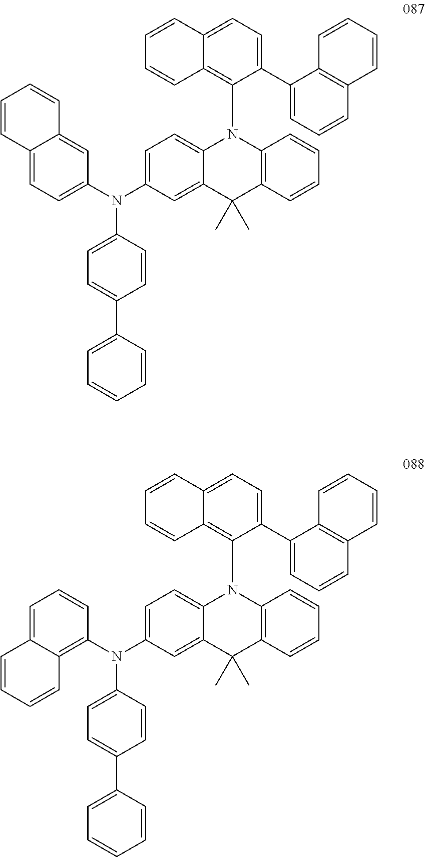 [SMILES: CC1(C)c2ccccc2N(c2c(-c3cccc4ccccc34)ccc3ccccc23)c2ccc(N(c3ccc(-c4ccccc4)cc3)c3ccc4ccccc4c3)cc21 ; CC1(C)c2ccccc2N(c2c(-c3cccc4ccccc34)ccc3ccccc23)c2ccc(N(c3ccc(-c4ccccc4)cc3)c3cccc4ccccc34)cc21]

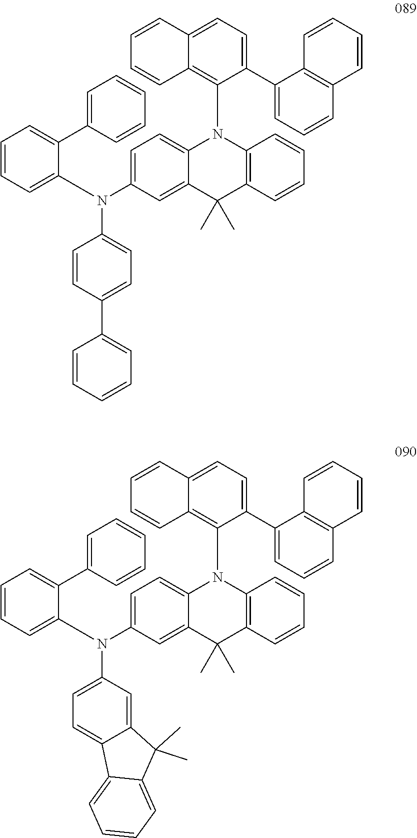 [SMILES: CC1(C)c2ccccc2N(c2c(-c3cccc4ccccc34)ccc3ccccc23)c2ccc(N(c3ccc(-c4ccccc4)cc3)c3ccccc3-c3ccccc3)cc21 ; CC1(C)c2ccccc2-c2ccc(N(c3ccc4c(c3)C(C)(C)c3ccccc3N4c3c(-c4cccc5ccccc45)ccc4ccccc34)c3ccccc3-c3ccccc3)cc21]

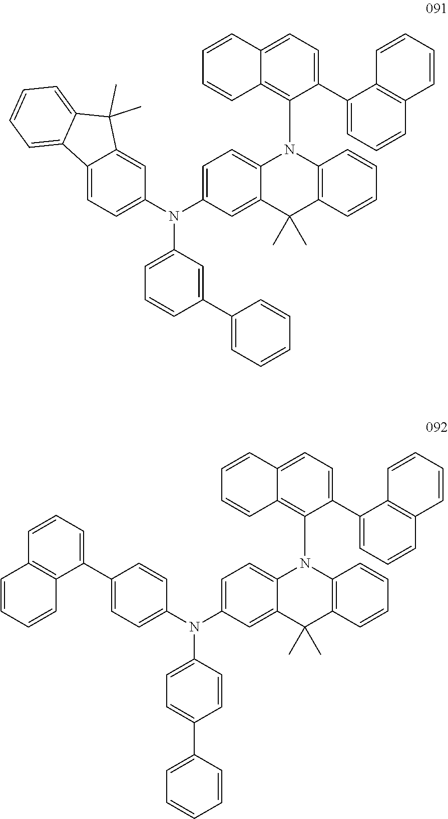 [SMILES: CC1(C)c2ccccc2-c2ccc(N(c3cccc(-c4ccccc4)c3)c3ccc4c(c3)C(C)(C)c3ccccc3N4c3c(-c4cccc5ccccc45)ccc4ccccc34)cc21 ; CC1(C)c2ccccc2N(c2c(-c3cccc4ccccc34)ccc3ccccc23)c2ccc(N(c3ccc(-c4ccccc4)cc3)c3ccc(-c4cccc5ccccc45)cc3)cc21]

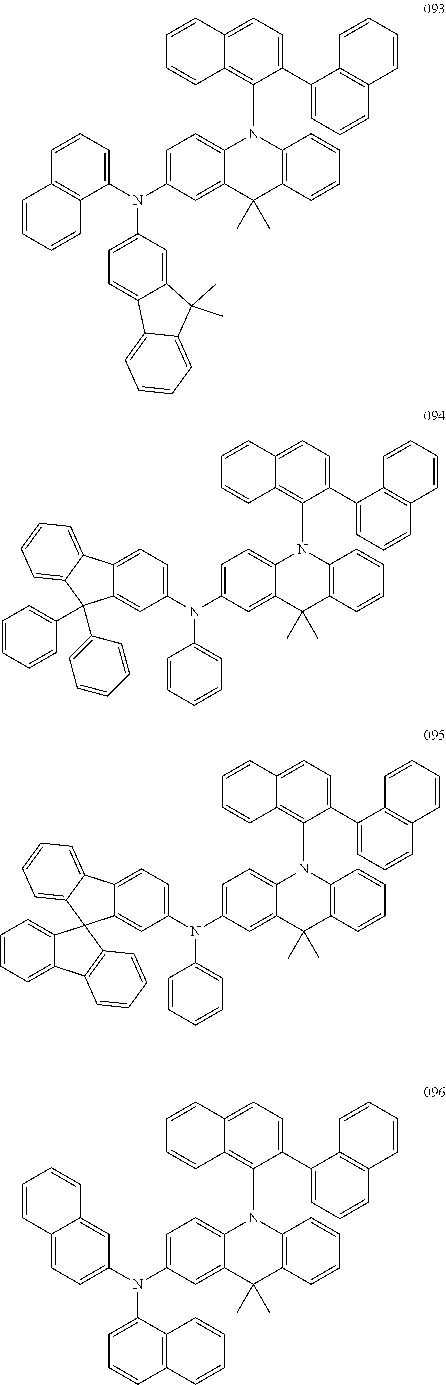 [SMILES: CC1(C)c2ccccc2-c2ccc(N(c3ccc4c(c3)C(C)(C)c3ccccc3N4c3c(-c4cccc5ccccc45)ccc4ccccc34)c3cccc4ccccc34)cc21 ; CC1(C)c2ccccc2N(c2c(-c3cccc4ccccc34)ccc3ccccc23)c2ccc(N(c3ccccc3)c3ccc4c(c3)C(c3ccccc3)(c3ccccc3)c3ccccc3-4)cc21 ; CC1(C)c2ccccc2N(c2c(-c3cccc4ccccc34)ccc3ccccc23)c2ccc(N(c3ccccc3)c3ccc4c(c3)C3(c5ccccc5-c5ccccc53)c3ccccc3-4)cc21 ; CC1(C)c2ccccc2N(c2c(-c3cccc4ccccc34)ccc3ccccc23)c2ccc(N(c3ccc4ccccc4c3)c3cccc4ccccc34)cc21]

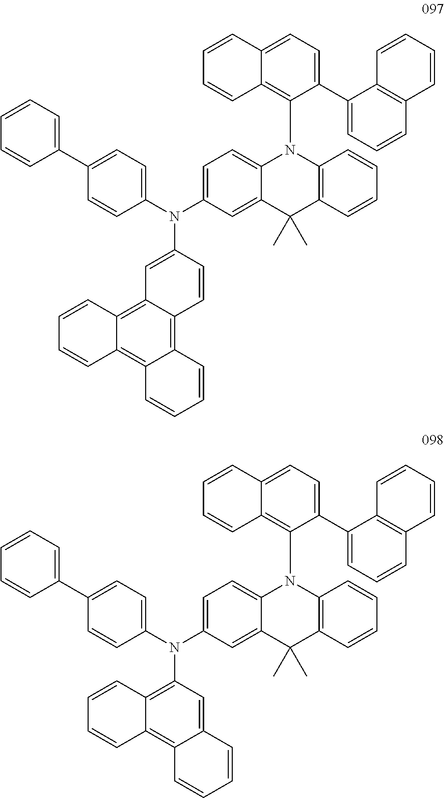 [SMILES: CC1(C)c2ccccc2N(c2c(-c3cccc4ccccc34)ccc3ccccc23)c2ccc(N(c3ccc(-c4ccccc4)cc3)c3ccc4c5ccccc5c5ccccc5c4c3)cc21 ; CC1(C)c2ccccc2N(c2c(-c3cccc4ccccc34)ccc3ccccc23)c2ccc(N(c3ccc(-c4ccccc4)cc3)c3cc4ccccc4c4ccccc34)cc21]

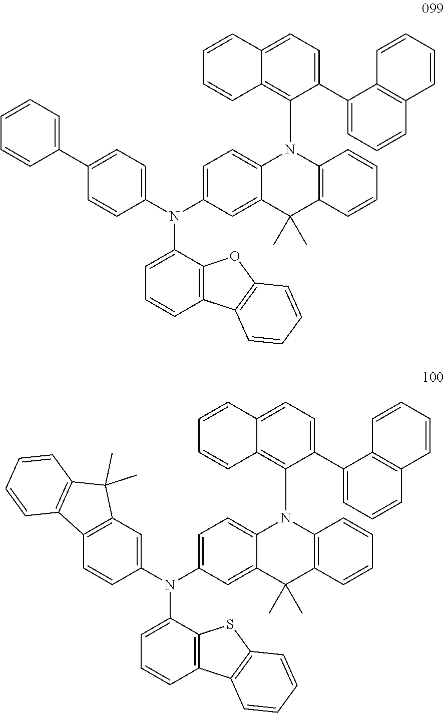 [SMILES: CC1(C)c2ccccc2N(c2c(-c3cccc4ccccc34)ccc3ccccc23)c2ccc(N(c3ccc(-c4ccccc4)cc3)c3cccc4c3oc3ccccc34)cc21 ; CC1(C)c2ccccc2-c2ccc(N(c3ccc4c(c3)C(C)(C)c3ccccc3N4c3c(-c4cccc5ccccc45)ccc4ccccc34)c3cccc4c3sc3ccccc34)cc21]

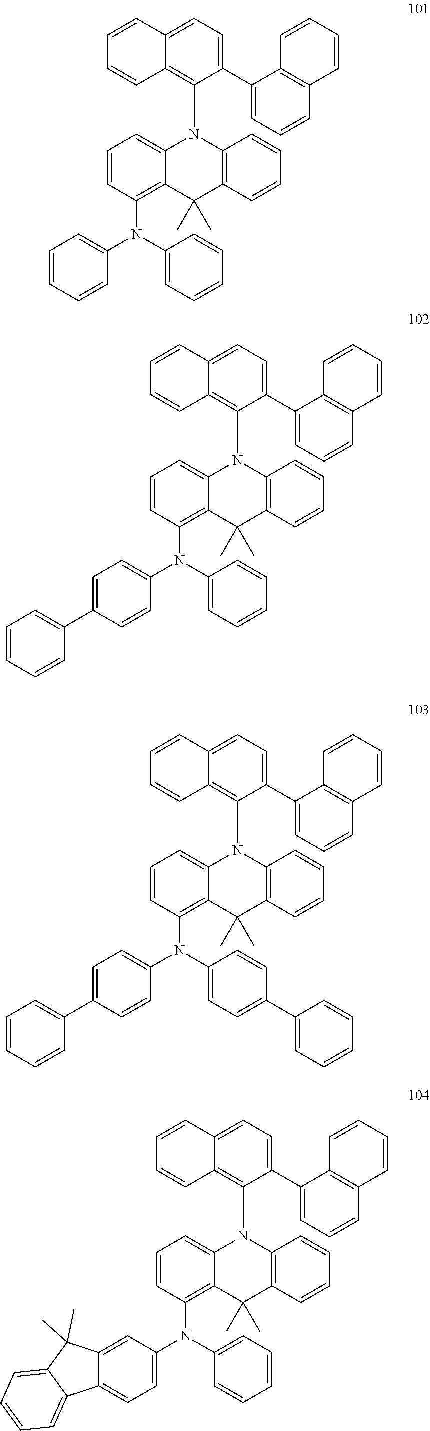

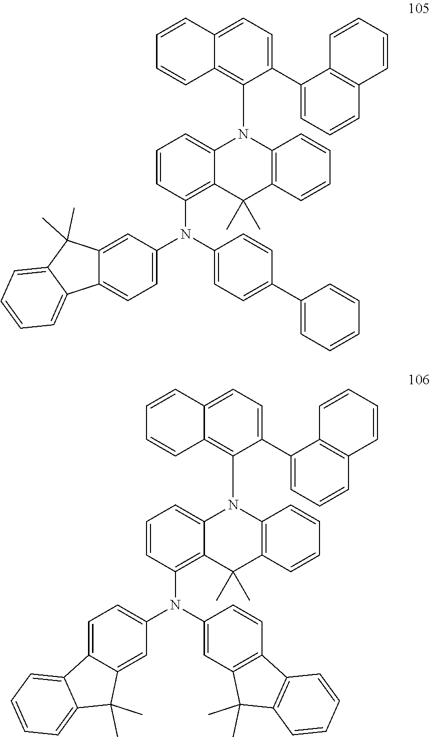

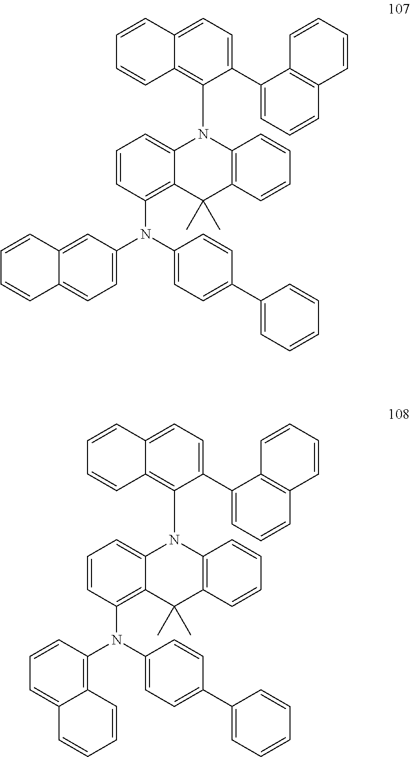 [SMILES: CC1(C)c2ccccc2N(c2c(-c3cccc4ccccc34)ccc3ccccc23)c2cccc(N(c3ccc(-c4ccccc4)cc3)c3ccc4ccccc4c3)c21 ; CC1(C)c2ccccc2N(c2c(-c3cccc4ccccc34)ccc3ccccc23)c2cccc(N(c3ccc(-c4ccccc4)cc3)c3cccc4ccccc34)c21]

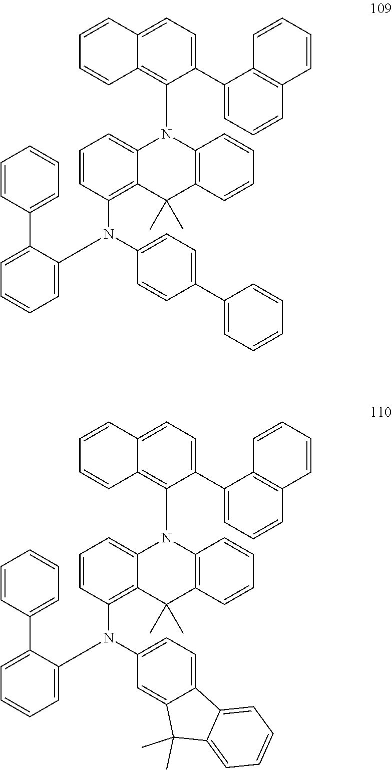 [SMILES: CC1(C)c2ccccc2N(c2c(-c3cccc4ccccc34)ccc3ccccc23)c2cccc(N(c3ccc(-c4ccccc4)cc3)c3ccccc3-c3ccccc3)c21 ; CC1(C)c2ccccc2-c2ccc(N(c3ccccc3-c3ccccc3)c3cccc4c3C(C)(C)c3ccccc3N4c3c(-c4cccc5ccccc45)ccc4ccccc34)cc21]

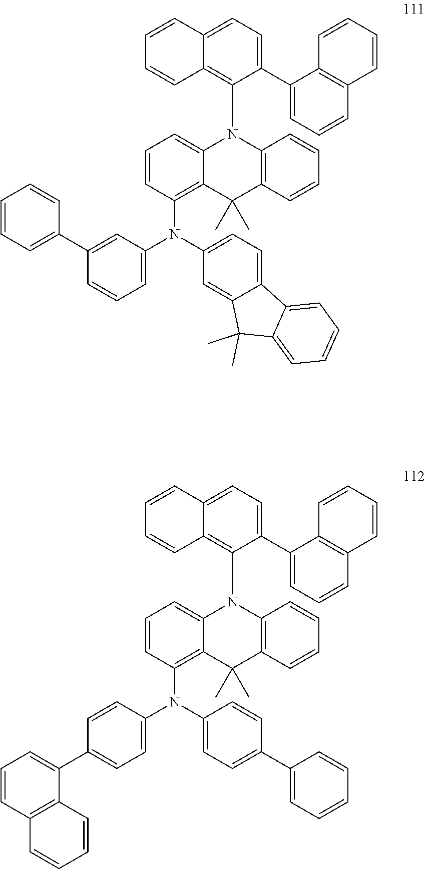 [SMILES: CC1(C)c2ccccc2-c2ccc(N(c3cccc(-c4ccccc4)c3)c3cccc4c3C(C)(C)c3ccccc3N4c3c(-c4cccc5ccccc45)ccc4ccccc34)cc21 ; CC1(C)c2ccccc2N(c2c(-c3cccc4ccccc34)ccc3ccccc23)c2cccc(N(c3ccc(-c4ccccc4)cc3)c3ccc(-c4cccc5ccccc45)cc3)c21]

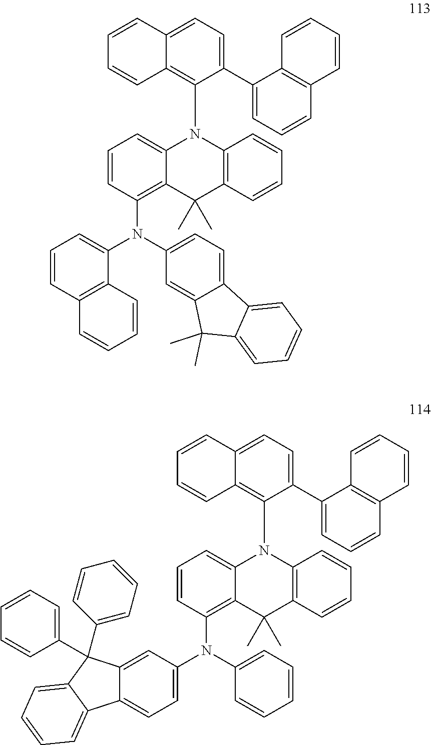 [SMILES: CC1(C)c2ccccc2-c2ccc(N(c3cccc4c3C(C)(C)c3ccccc3N4c3c(-c4cccc5ccccc45)ccc4ccccc34)c3cccc4ccccc34)cc21 ; CC1(C)c2ccccc2N(c2c(-c3cccc4ccccc34)ccc3ccccc23)c2cccc(N(c3ccccc3)c3ccc4c(c3)C(c3ccccc3)(c3ccccc3)c3ccccc3-4)c21]

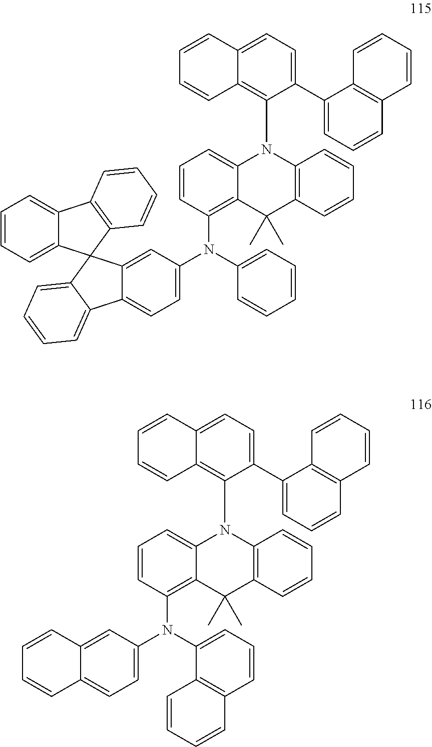 [SMILES: CC1(C)c2ccccc2N(c2c(-c3cccc4ccccc34)ccc3ccccc23)c2cccc(N(c3ccccc3)c3ccc4c(c3)C3(c5ccccc5-c5ccccc53)c3ccccc3-4)c21 ; CC1(C)c2ccccc2N(c2c(-c3cccc4ccccc34)ccc3ccccc23)c2cccc(N(c3ccc4ccccc4c3)c3cccc4ccccc34)c21]

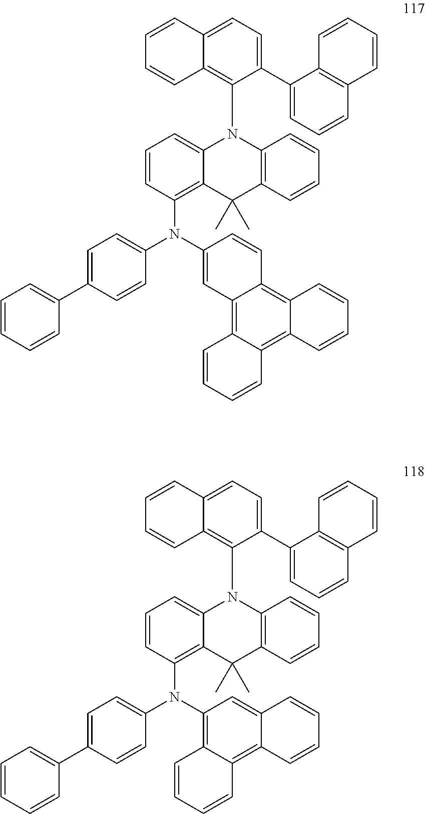 [SMILES: CC1(C)c2ccccc2N(c2c(-c3cccc4ccccc34)ccc3ccccc23)c2cccc(N(c3ccc(-c4ccccc4)cc3)c3ccc4c5ccccc5c5ccccc5c4c3)c21 ; CC1(C)c2ccccc2N(c2c(-c3cccc4ccccc34)ccc3ccccc23)c2cccc(N(c3ccc(-c4ccccc4)cc3)c3cc4ccccc4c4ccccc34)c21]

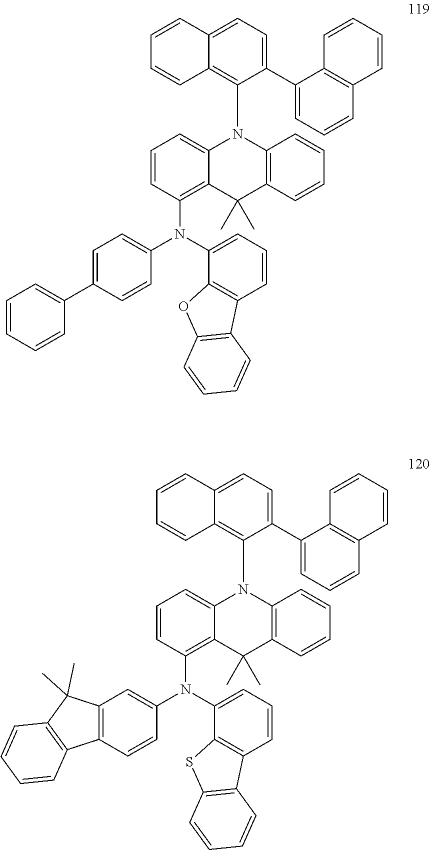 [SMILES: CC1(C)c2ccccc2N(c2c(-c3cccc4ccccc34)ccc3ccccc23)c2cccc(N(c3ccc(-c4ccccc4)cc3)c3cccc4c3oc3ccccc34)c21 ; CC1(C)c2ccccc2-c2ccc(N(c3cccc4c3C(C)(C)c3ccccc3N4c3c(-c4cccc5ccccc45)ccc4ccccc34)c3cccc4c3sc3ccccc34)cc21]

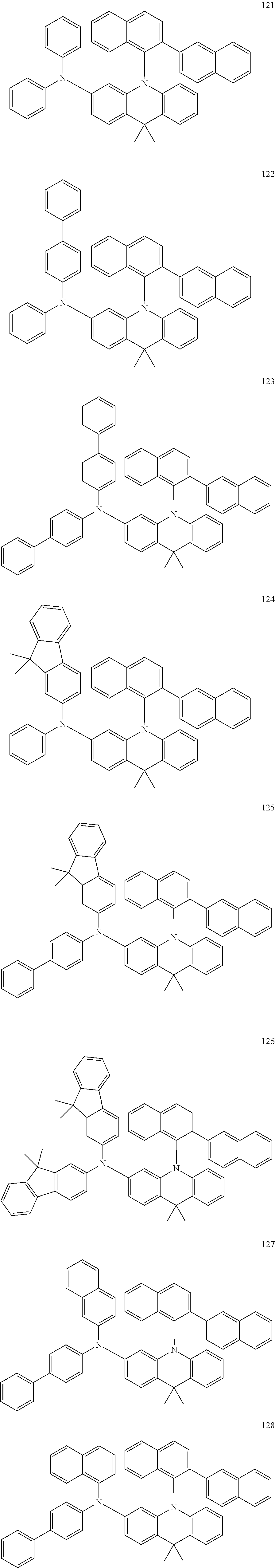

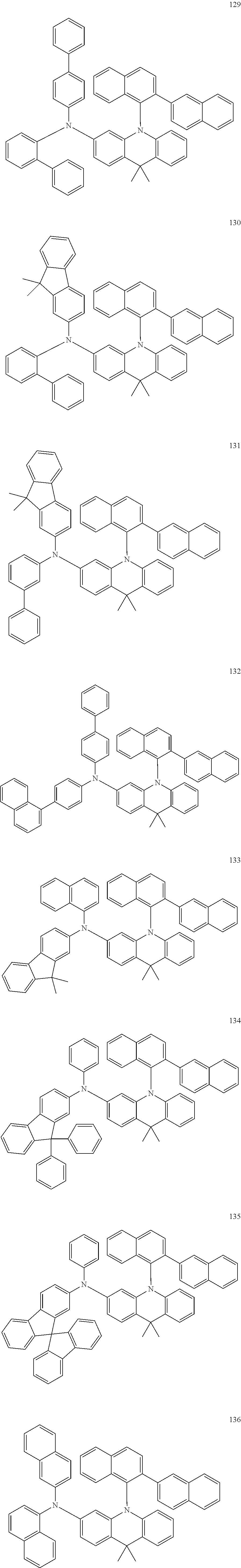

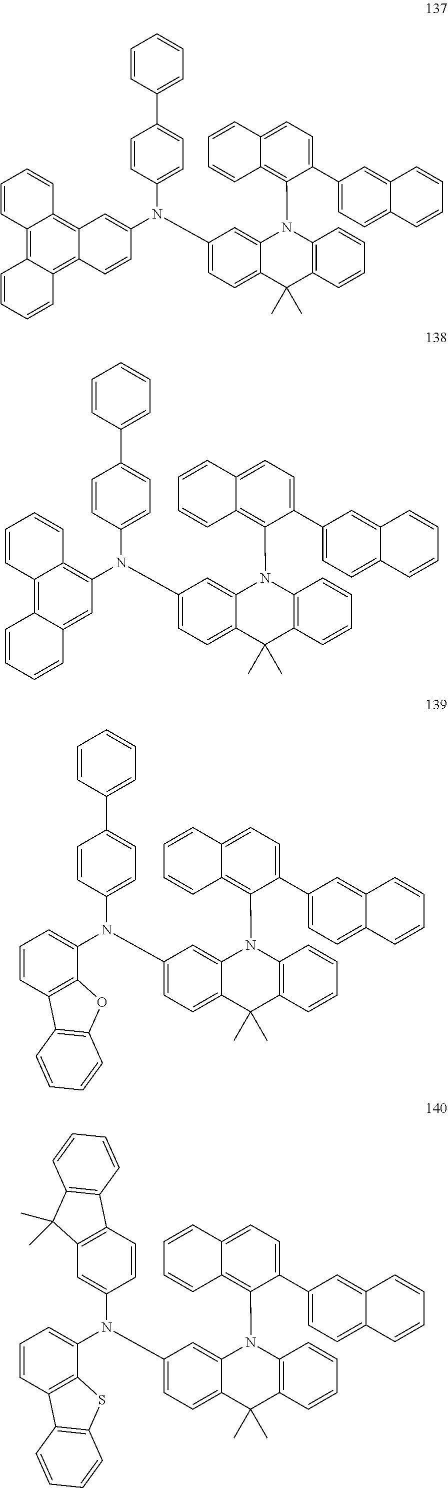

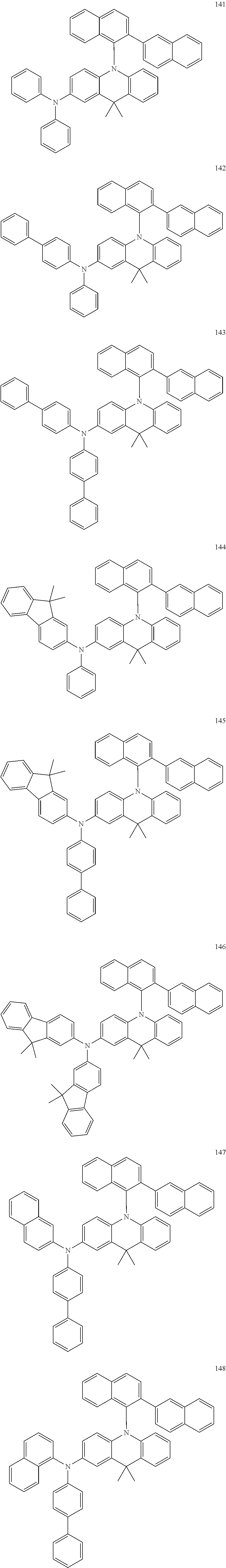

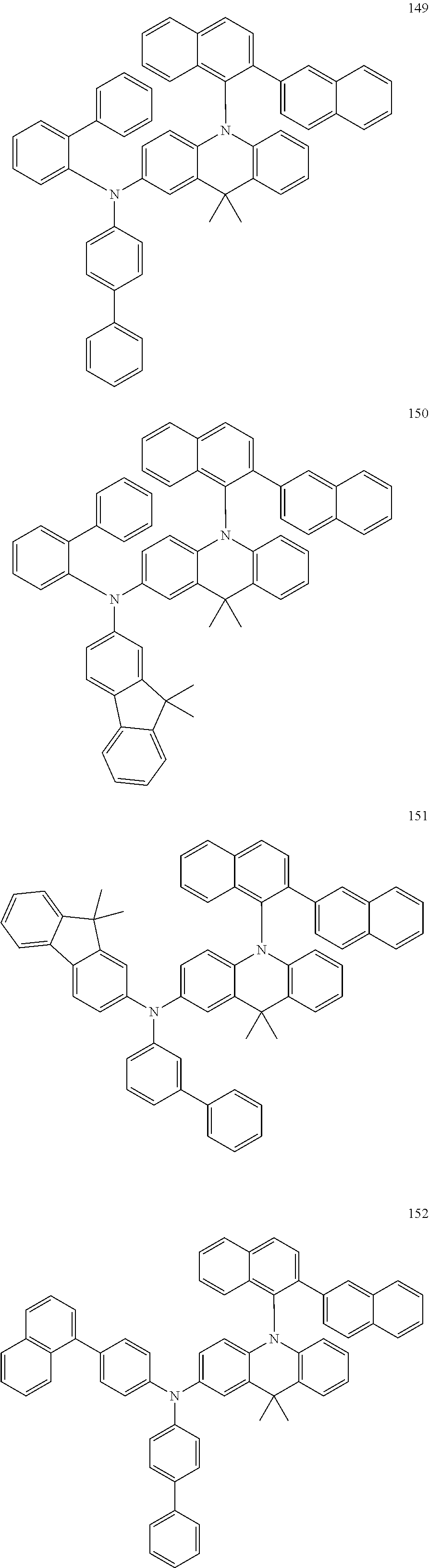 [SMILES: CC1(C)c2ccccc2N(c2c(-c3ccc4ccccc4c3)ccc3ccccc23)c2ccc(N(c3ccc(-c4ccccc4)cc3)c3ccccc3-c3ccccc3)cc21 ; CC1(C)c2ccccc2-c2ccc(N(c3ccc4c(c3)C(C)(C)c3ccccc3N4c3c(-c4ccc5ccccc5c4)ccc4ccccc34)c3ccccc3-c3ccccc3)cc21 ; CC1(C)c2ccccc2-c2ccc(N(c3cccc(-c4ccccc4)c3)c3ccc4c(c3)C(C)(C)c3ccccc3N4c3c(-c4ccc5ccccc5c4)ccc4ccccc34)cc21 ; CC1(C)c2ccccc2N(c2c(-c3ccc4ccccc4c3)ccc3ccccc23)c2ccc(N(c3ccc(-c4ccccc4)cc3)c3ccc(-c4cccc5ccccc45)cc3)cc21]

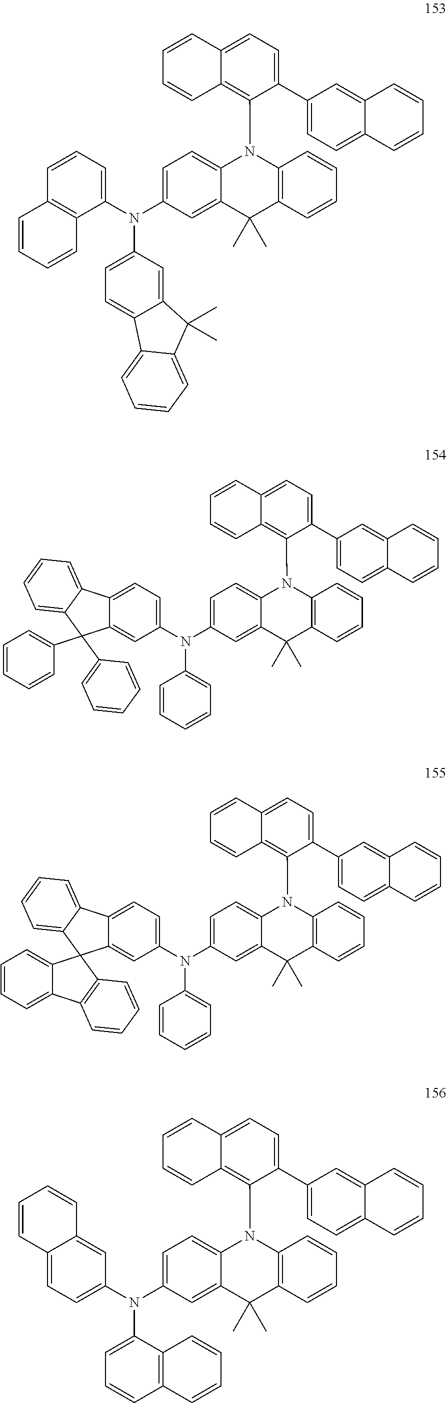 [SMILES: CC1(C)c2ccccc2-c2ccc(N(c3ccc4c(c3)C(C)(C)c3ccccc3N4c3c(-c4ccc5ccccc5c4)ccc4ccccc34)c3cccc4ccccc34)cc21 ; CC1(C)c2ccccc2N(c2c(-c3ccc4ccccc4c3)ccc3ccccc23)c2ccc(N(c3ccccc3)c3ccc4c(c3)C(c3ccccc3)(c3ccccc3)c3ccccc3-4)cc21 ; CC1(C)c2ccccc2N(c2c(-c3ccc4ccccc4c3)ccc3ccccc23)c2ccc(N(c3ccccc3)c3ccc4c(c3)C3(c5ccccc5-c5ccccc53)c3ccccc3-4)cc21 ; CC1(C)c2ccccc2N(c2c(-c3ccc4ccccc4c3)ccc3ccccc23)c2ccc(N(c3ccc4ccccc4c3)c3cccc4ccccc34)cc21]

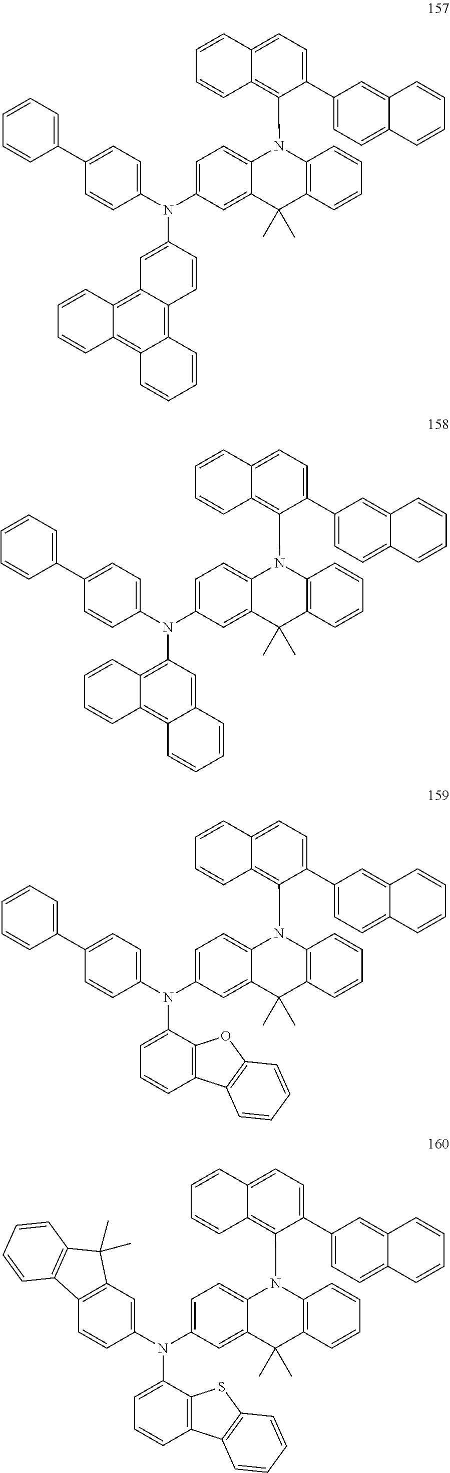 [SMILES: CC1(C)c2ccccc2N(c2c(-c3ccc4ccccc4c3)ccc3ccccc23)c2ccc(N(c3ccc(-c4ccccc4)cc3)c3ccc4c5ccccc5c5ccccc5c4c3)cc21 ; CC1(C)c2ccccc2N(c2c(-c3ccc4ccccc4c3)ccc3ccccc23)c2ccc(N(c3ccc(-c4ccccc4)cc3)c3cc4ccccc4c4ccccc34)cc21 ; CC1(C)c2ccccc2N(c2c(-c3ccc4ccccc4c3)ccc3ccccc23)c2ccc(N(c3ccc(-c4ccccc4)cc3)c3cccc4c3oc3ccccc34)cc21 ; CC1(C)c2ccccc2-c2ccc(N(c3ccc4c(c3)C(C)(C)c3ccccc3N4c3c(-c4ccc5ccccc5c4)ccc4ccccc34)c3cccc4c3sc3ccccc34)cc21]

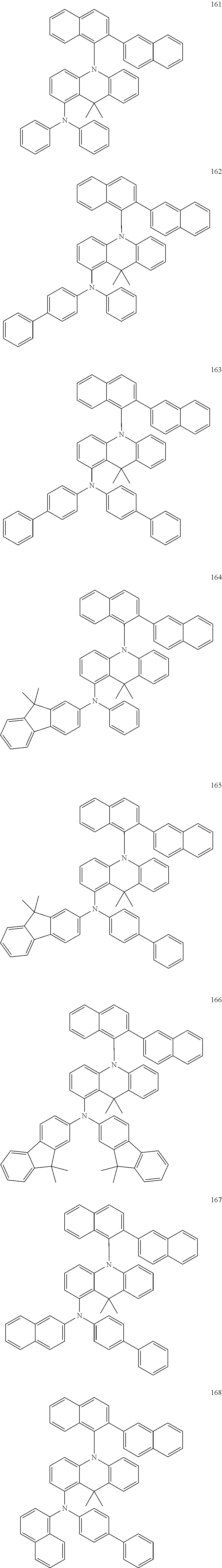

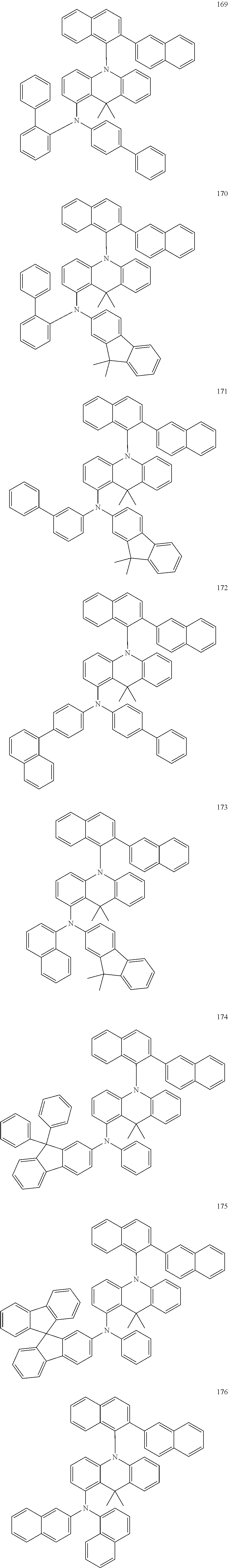

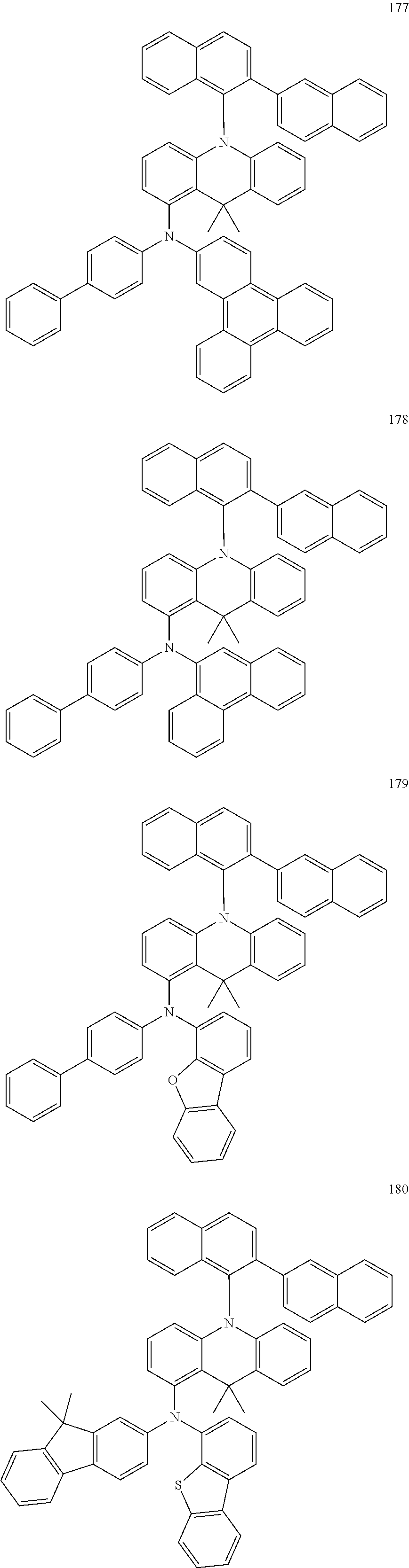 [SMILES: CC1(C)c2ccccc2N(c2c(-c3ccc4ccccc4c3)ccc3ccccc23)c2cccc(N(c3ccc(-c4ccccc4)cc3)c3ccc4c5ccccc5c5ccccc5c4c3)c21 ; CC1(C)c2ccccc2N(c2c(-c3ccc4ccccc4c3)ccc3ccccc23)c2cccc(N(c3ccc(-c4ccccc4)cc3)c3cc4ccccc4c4ccccc34)c21 ; CC1(C)c2ccccc2N(c2c(-c3ccc4ccccc4c3)ccc3ccccc23)c2cccc(N(c3ccc(-c4ccccc4)cc3)c3cccc4c3oc3ccccc34)c21 ; CC1(C)c2ccccc2-c2ccc(N(c3cccc4c3C(C)(C)c3ccccc3N4c3c(-c4ccc5ccccc5c4)ccc4ccccc34)c3cccc4c3sc3ccccc34)cc21]

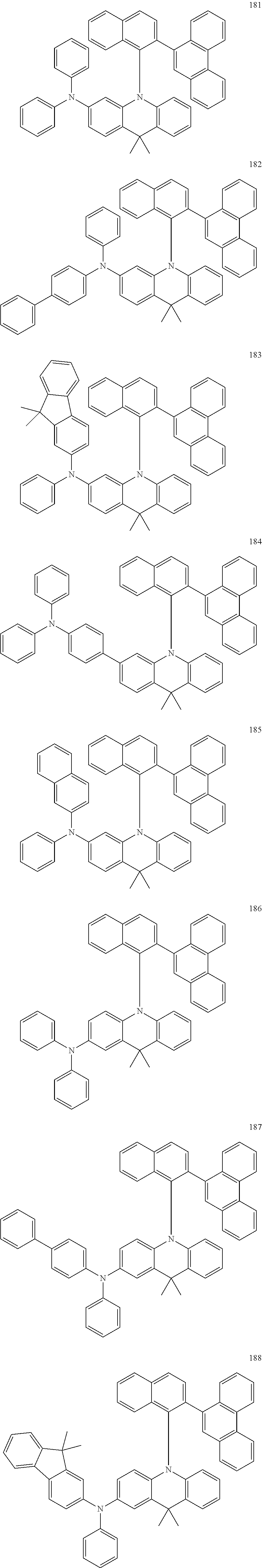

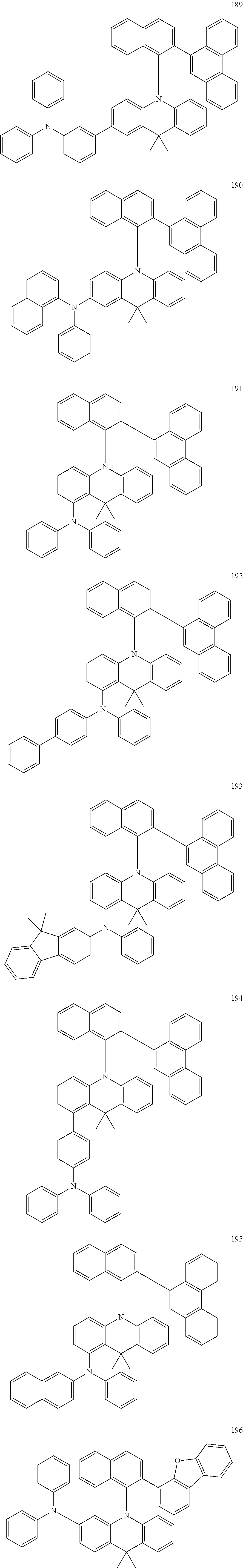

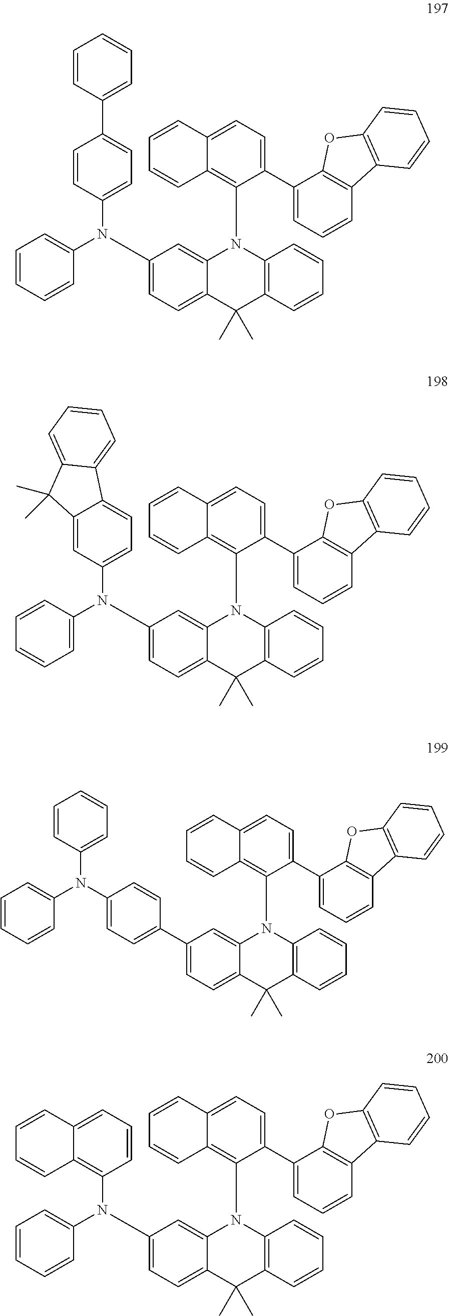

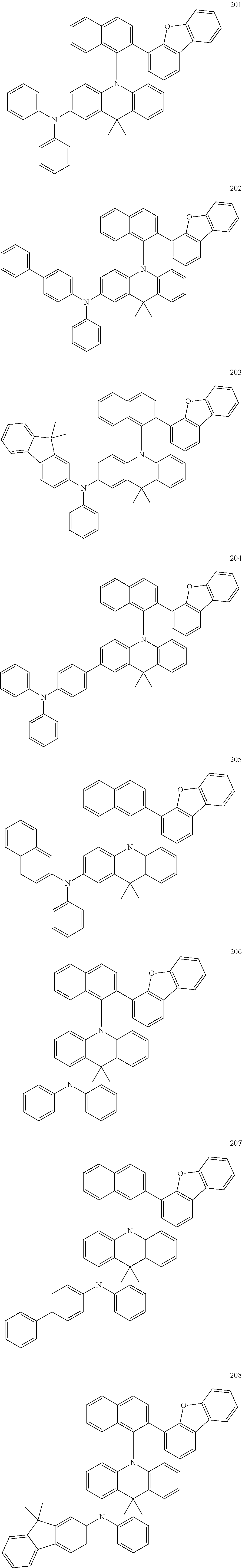

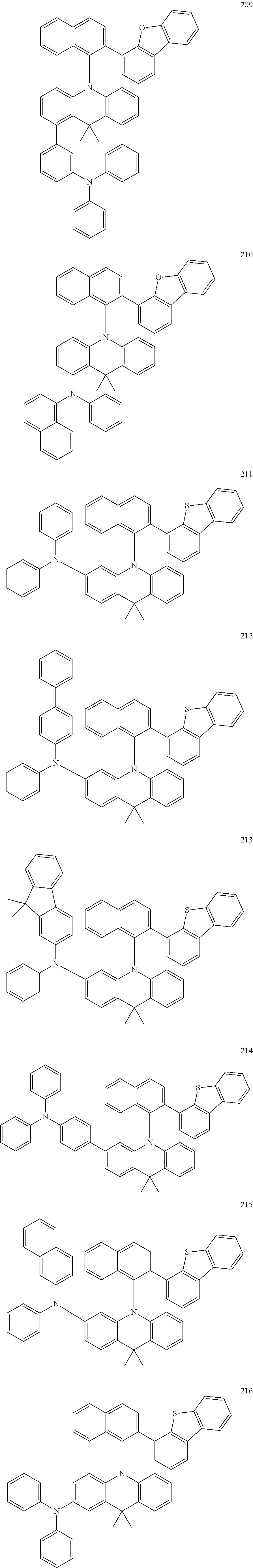

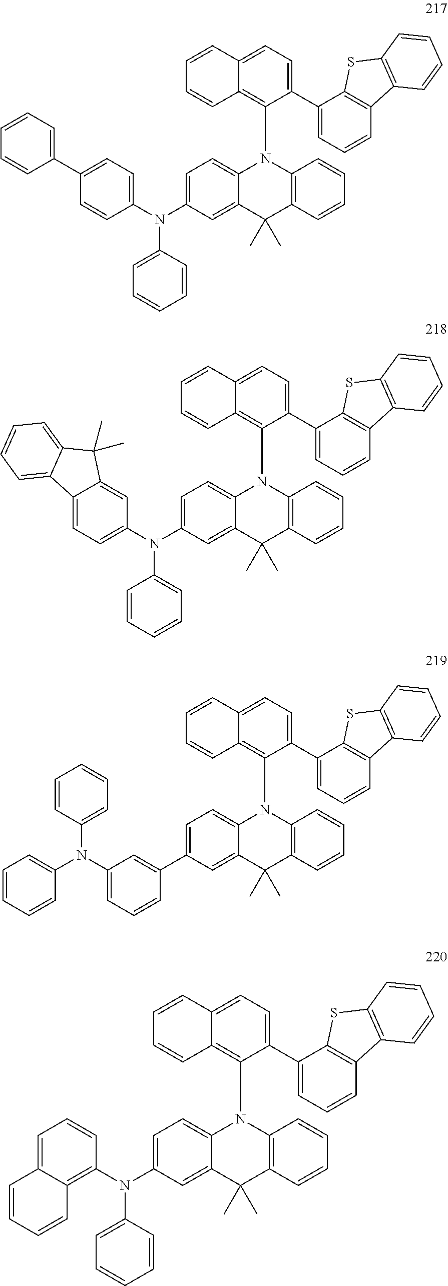

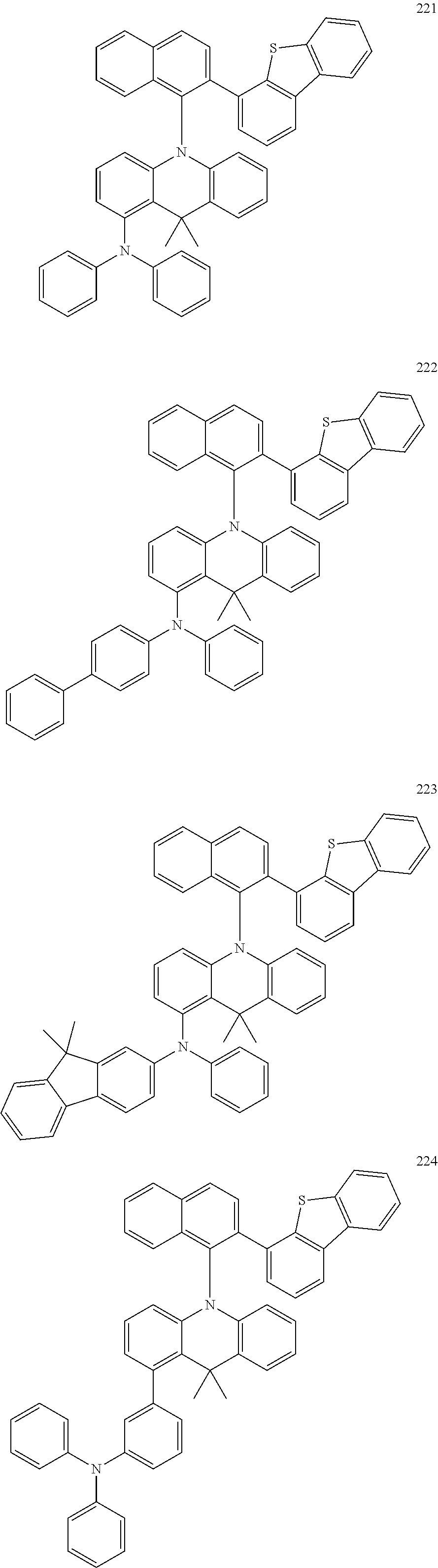 [SMILES: CC1(C)c2ccccc2N(c2c(-c3cccc4c3sc3ccccc34)ccc3ccccc23)c2cccc(N(c3ccccc3)c3ccccc3)c21 ; CC1(C)c2ccccc2N(c2c(-c3cccc4c3sc3ccccc34)ccc3ccccc23)c2cccc(N(c3ccccc3)c3ccc(-c4ccccc4)cc3)c21 ; CC1(C)c2ccccc2-c2ccc(N(c3ccccc3)c3cccc4c3C(C)(C)c3ccccc3N4c3c(-c4cccc5c4sc4ccccc45)ccc4ccccc34)cc21 ; CC1(C)c2ccccc2N(c2c(-c3cccc4c3sc3ccccc34)ccc3ccccc23)c2cccc(-c3cccc(N(c4ccccc4)c4ccccc4)c3)c21]

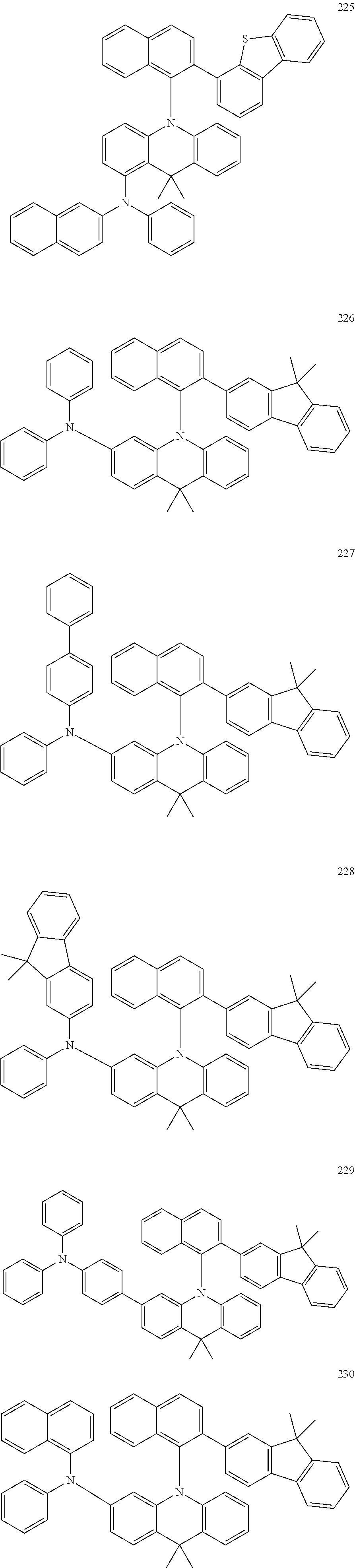

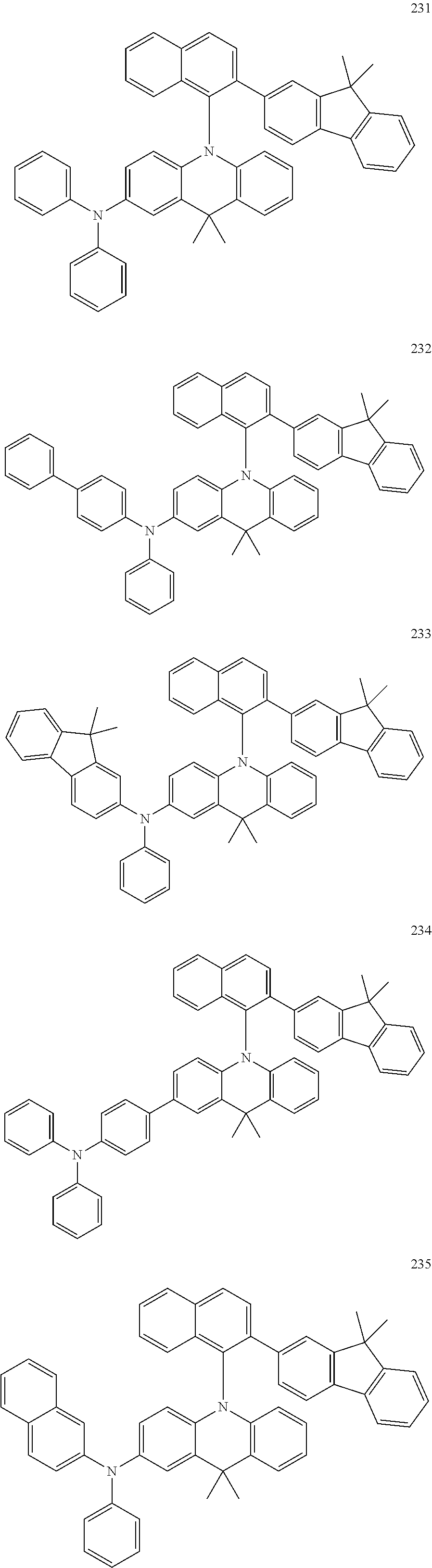 [SMILES: CC1(C)c2ccccc2-c2ccc(-c3ccc4ccccc4c3N3c4ccccc4C(C)(C)c4cc(N(c5ccccc5)c5ccccc5)ccc43)cc21 ; CC1(C)c2ccccc2-c2ccc(-c3ccc4ccccc4c3N3c4ccccc4C(C)(C)c4cc(N(c5ccccc5)c5ccc(-c6ccccc6)cc5)ccc43)cc21 ; CC1(C)c2ccccc2-c2ccc(-c3ccc4ccccc4c3N3c4ccccc4C(C)(C)c4cc(N(c5ccccc5)c5ccc6c(c5)C(C)(C)c5ccccc5-6)ccc43)cc21 ; CC1(C)c2ccccc2-c2ccc(-c3ccc4ccccc4c3N3c4ccccc4C(C)(C)c4cc(-c5ccc(N(c6ccccc6)c6ccccc6)cc5)ccc43)cc21 ; CC1(C)c2ccccc2-c2ccc(-c3ccc4ccccc4c3N3c4ccccc4C(C)(C)c4cc(N(c5ccccc5)c5ccc6ccccc6c5)ccc43)cc21]

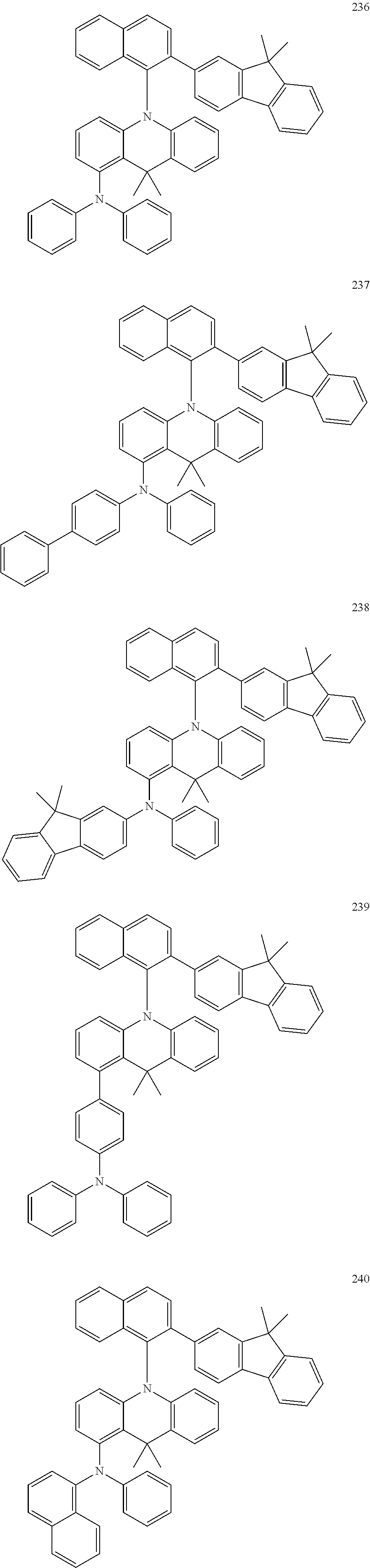 [SMILES: CC1(C)c2ccccc2-c2ccc(-c3ccc4ccccc4c3N3c4ccccc4C(C)(C)c4c(N(c5ccccc5)c5ccccc5)cccc43)cc21 ; CC1(C)c2ccccc2-c2ccc(-c3ccc4ccccc4c3N3c4ccccc4C(C)(C)c4c(N(c5ccccc5)c5ccc(-c6ccccc6)cc5)cccc43)cc21 ; CC1(C)c2ccccc2-c2ccc(-c3ccc4ccccc4c3N3c4ccccc4C(C)(C)c4c(N(c5ccccc5)c5ccc6c(c5)C(C)(C)c5ccccc5-6)cccc43)cc21 ; CC1(C)c2ccccc2-c2ccc(-c3ccc4ccccc4c3N3c4ccccc4C(C)(C)c4c(-c5ccc(N(c6ccccc6)c6ccccc6)cc5)cccc43)cc21 ; CC1(C)c2ccccc2-c2ccc(-c3ccc4ccccc4c3N3c4ccccc4C(C)(C)c4c(N(c5ccccc5)c5cccc6ccccc56)cccc43)cc21]

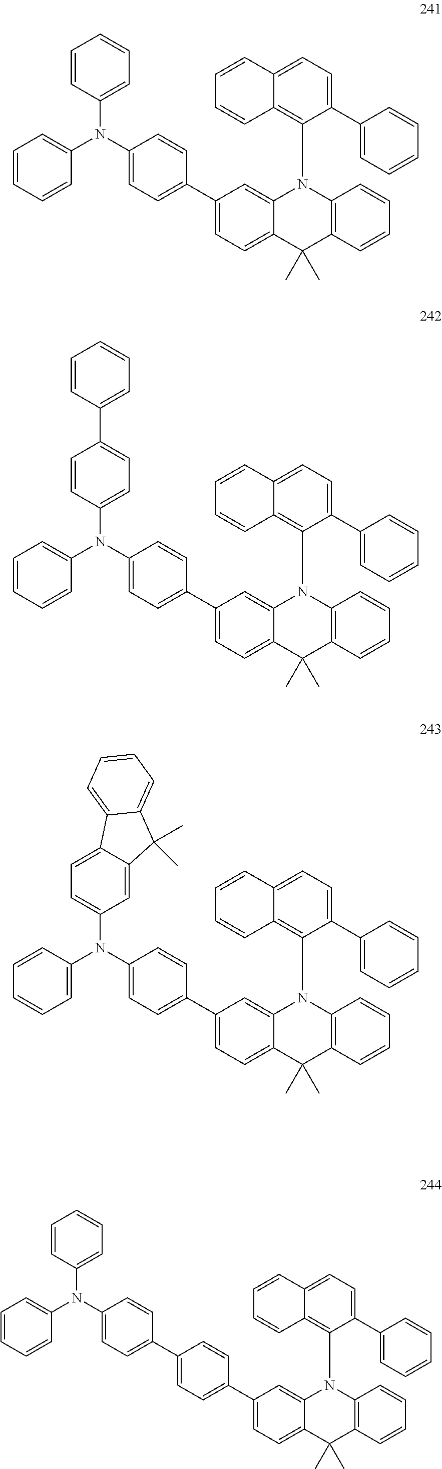 [SMILES: CC1(C)c2ccccc2N(c2c(-c3ccccc3)ccc3ccccc23)c2cc(-c3ccc(N(c4ccccc4)c4ccccc4)cc3)ccc21 ; CC1(C)c2ccccc2N(c2c(-c3ccccc3)ccc3ccccc23)c2cc(-c3ccc(N(c4ccccc4)c4ccc(-c5ccccc5)cc4)cc3)ccc21 ; CC1(C)c2ccccc2-c2ccc(N(c3ccccc3)c3ccc(-c4ccc5c(c4)N(c4c(-c6ccccc6)ccc6ccccc46)c4ccccc4C5(C)C)cc3)cc21 ; CC1(C)c2ccccc2N(c2c(-c3ccccc3)ccc3ccccc23)c2cc(-c3ccc(-c4ccc(N(c5ccccc5)c5ccccc5)cc4)cc3)ccc21]

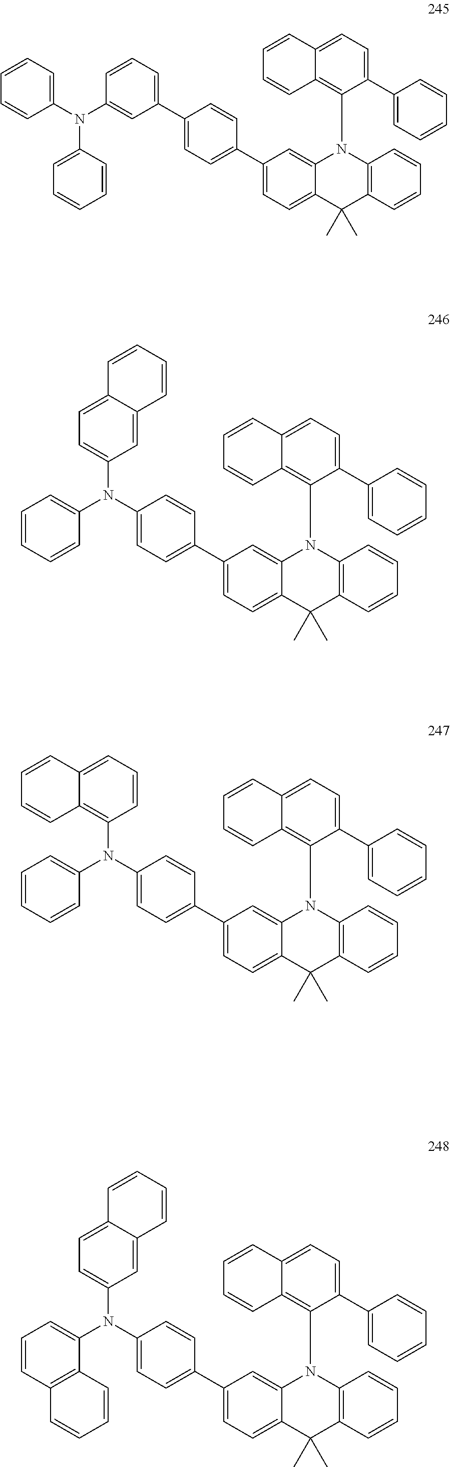 [SMILES: CC1(C)c2ccccc2N(c2c(-c3ccccc3)ccc3ccccc23)c2cc(-c3ccc(-c4cccc(N(c5ccccc5)c5ccccc5)c4)cc3)ccc21 ; CC1(C)c2ccccc2N(c2c(-c3ccccc3)ccc3ccccc23)c2cc(-c3ccc(N(c4ccccc4)c4ccc5ccccc5c4)cc3)ccc21 ; CC1(C)c2ccccc2N(c2c(-c3ccccc3)ccc3ccccc23)c2cc(-c3ccc(N(c4ccccc4)c4cccc5ccccc45)cc3)ccc21 ; CC1(C)c2ccccc2N(c2c(-c3ccccc3)ccc3ccccc23)c2cc(-c3ccc(N(c4ccc5ccccc5c4)c4cccc5ccccc45)cc3)ccc21]

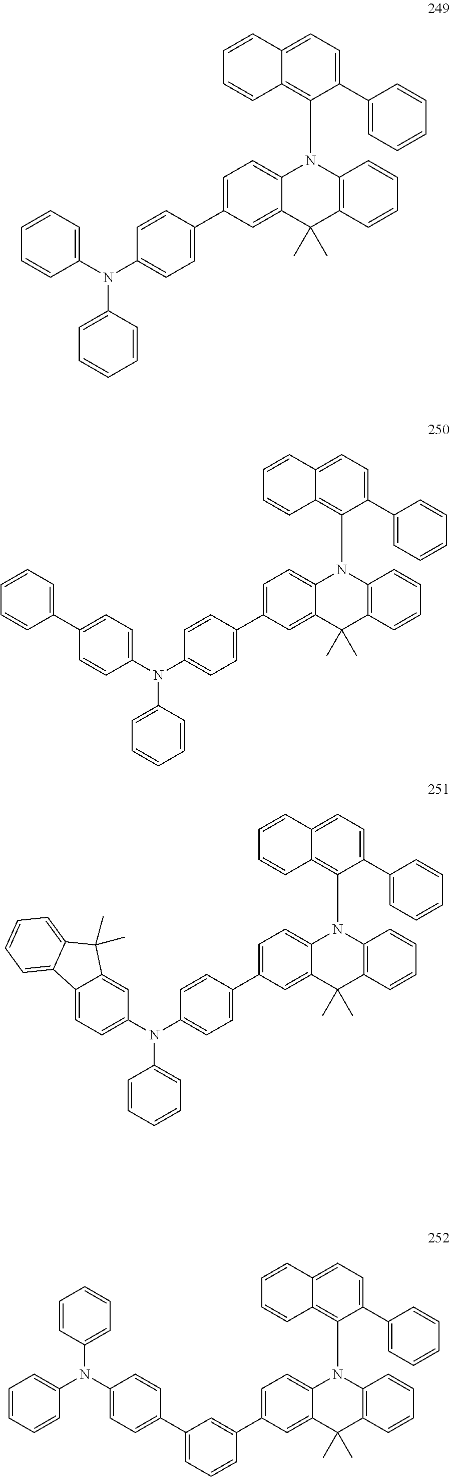 [SMILES: CC1(C)c2ccccc2N(c2c(-c3ccccc3)ccc3ccccc23)c2ccc(-c3ccc(N(c4ccccc4)c4ccccc4)cc3)cc21 ; CC1(C)c2ccccc2N(c2c(-c3ccccc3)ccc3ccccc23)c2ccc(-c3ccc(N(c4ccccc4)c4ccc(-c5ccccc5)cc4)cc3)cc21 ; CC1(C)c2ccccc2-c2ccc(N(c3ccccc3)c3ccc(-c4ccc5c(c4)C(C)(C)c4ccccc4N5c4c(-c5ccccc5)ccc5ccccc45)cc3)cc21 ; CC1(C)c2ccccc2N(c2c(-c3ccccc3)ccc3ccccc23)c2ccc(-c3cccc(-c4ccc(N(c5ccccc5)c5ccccc5)cc4)c3)cc21]

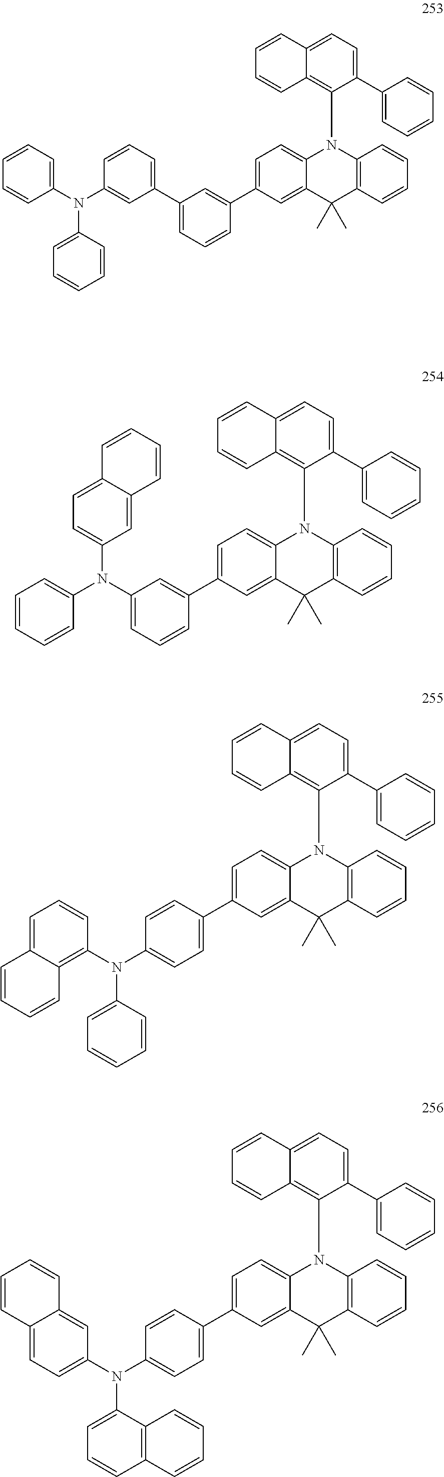 [SMILES: CC1(C)c2ccccc2N(c2c(-c3ccccc3)ccc3ccccc23)c2ccc(-c3cccc(-c4cccc(N(c5ccccc5)c5ccccc5)c4)c3)cc21 ; CC1(C)c2ccccc2N(c2c(-c3ccccc3)ccc3ccccc23)c2ccc(-c3cccc(N(c4ccccc4)c4ccc5ccccc5c4)c3)cc21 ; CC1(C)c2ccccc2N(c2c(-c3ccccc3)ccc3ccccc23)c2ccc(-c3ccc(N(c4ccccc4)c4cccc5ccccc45)cc3)cc21 ; CC1(C)c2ccccc2N(c2c(-c3ccccc3)ccc3ccccc23)c2ccc(-c3ccc(N(c4ccc5ccccc5c4)c4cccc5ccccc45)cc3)cc21]

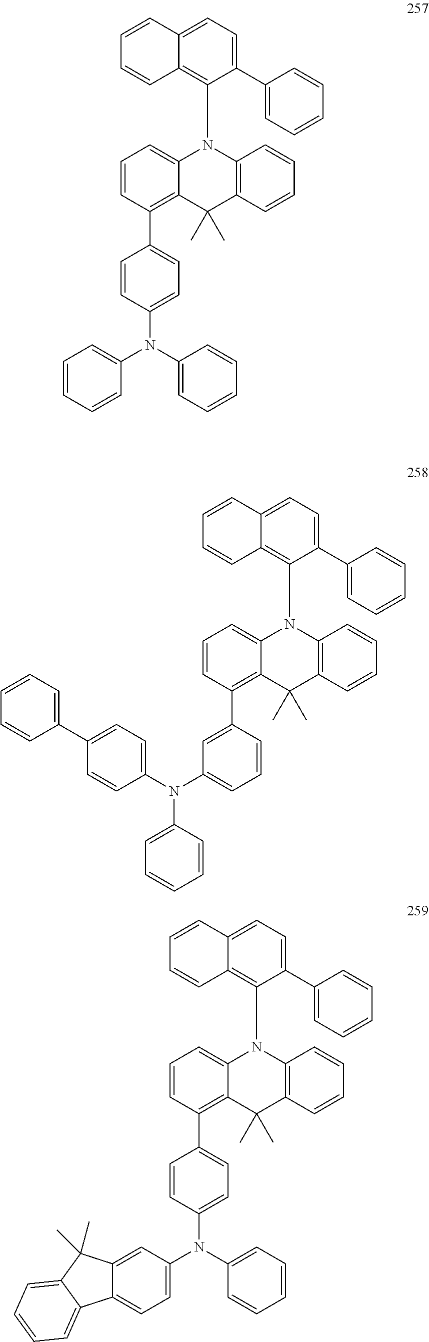

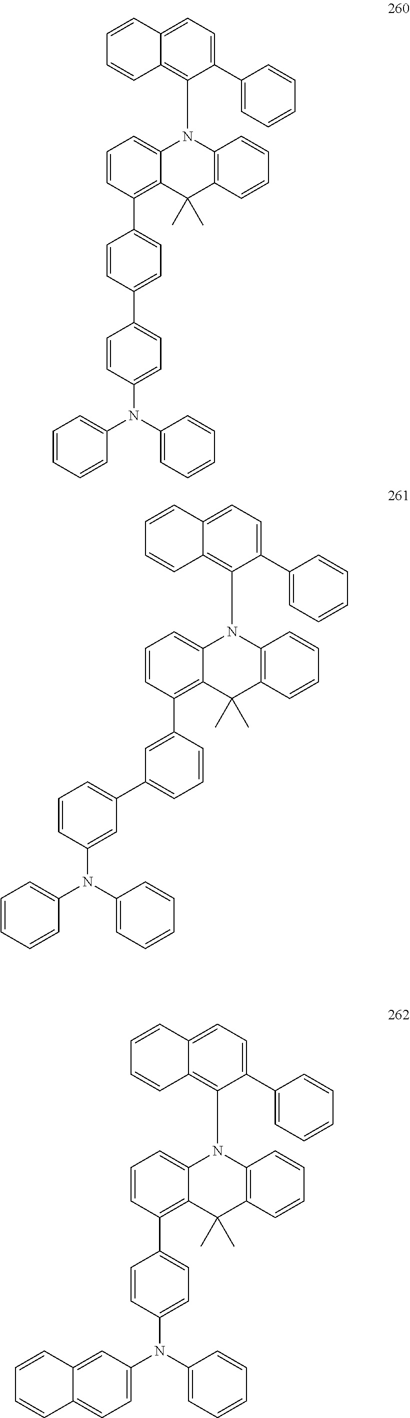

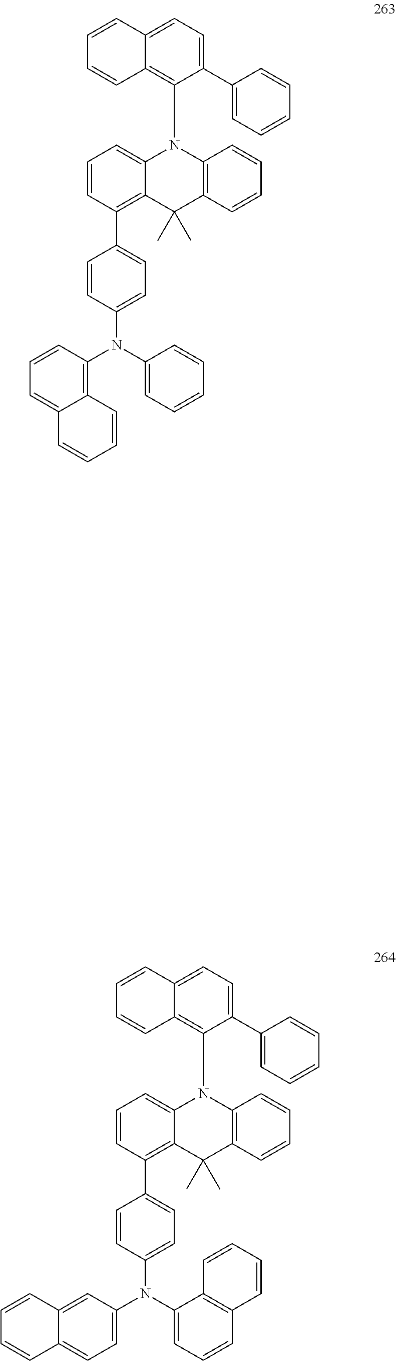 [SMILES: CC1(C)c2ccccc2N(c2c(-c3ccccc3)ccc3ccccc23)c2cccc(-c3ccc(N(c4ccccc4)c4cccc5ccccc45)cc3)c21 ; CC1(C)c2ccccc2N(c2c(-c3ccccc3)ccc3ccccc23)c2cccc(-c3ccc(N(c4ccc5ccccc5c4)c4cccc5ccccc45)cc3)c21]

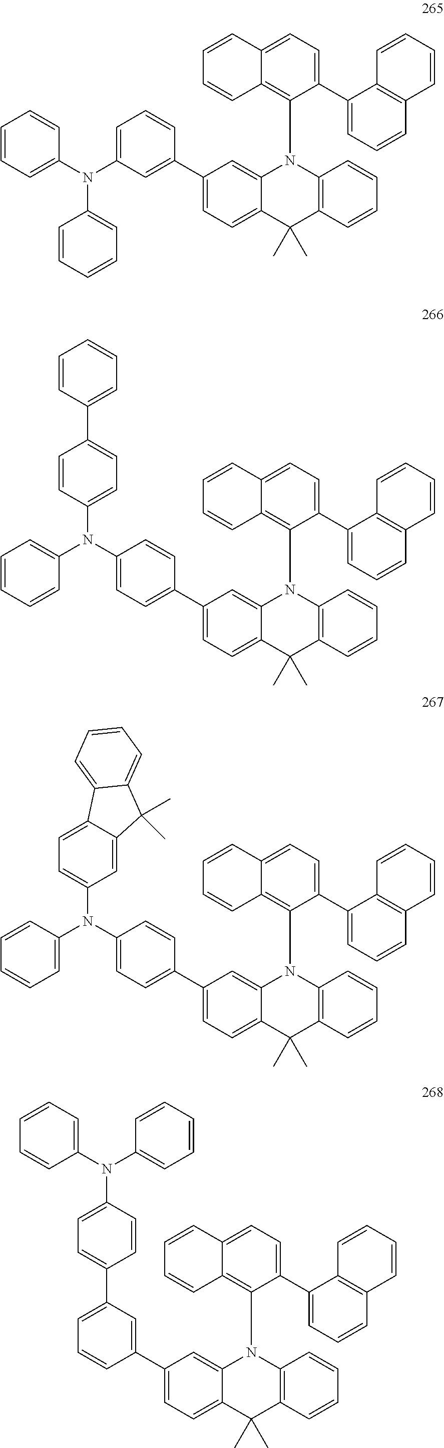 [SMILES: CC1(C)c2ccccc2N(c2c(-c3cccc4ccccc34)ccc3ccccc23)c2cc(-c3cccc(N(c4ccccc4)c4ccccc4)c3)ccc21 ; CC1(C)c2ccccc2N(c2c(-c3cccc4ccccc34)ccc3ccccc23)c2cc(-c3ccc(N(c4ccccc4)c4ccc(-c5ccccc5)cc4)cc3)ccc21 ; CC1(C)c2ccccc2-c2ccc(N(c3ccccc3)c3ccc(-c4ccc5c(c4)N(c4c(-c6cccc7ccccc67)ccc6ccccc46)c4ccccc4C5(C)C)cc3)cc21 ; CC1(C)c2ccccc2N(c2c(-c3cccc4ccccc34)ccc3ccccc23)c2cc(-c3cccc(-c4ccc(N(c5ccccc5)c5ccccc5)cc4)c3)ccc21]

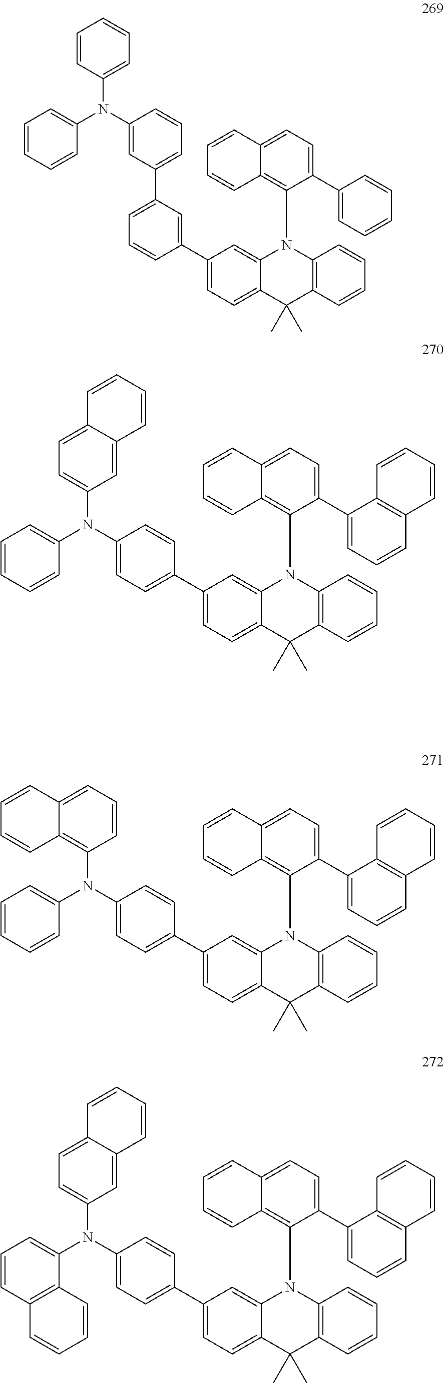 [SMILES: CC1(C)c2ccccc2N(c2c(-c3ccccc3)ccc3ccccc23)c2cc(-c3cccc(-c4cccc(N(c5ccccc5)c5ccccc5)c4)c3)ccc21 ; CC1(C)c2ccccc2N(c2c(-c3cccc4ccccc34)ccc3ccccc23)c2cc(-c3ccc(N(c4ccccc4)c4ccc5ccccc5c4)cc3)ccc21 ; CC1(C)c2ccccc2N(c2c(-c3cccc4ccccc34)ccc3ccccc23)c2cc(-c3ccc(N(c4ccccc4)c4cccc5ccccc45)cc3)ccc21 ; CC1(C)c2ccccc2N(c2c(-c3cccc4ccccc34)ccc3ccccc23)c2cc(-c3ccc(N(c4ccc5ccccc5c4)c4cccc5ccccc45)cc3)ccc21]

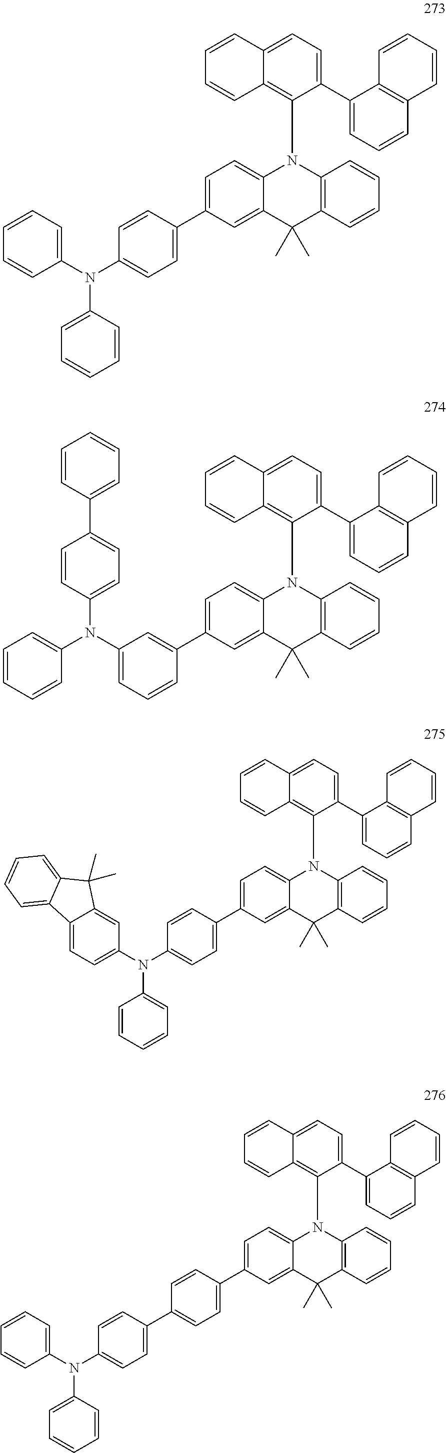 [SMILES: CC1(C)c2ccccc2N(c2c(-c3cccc4ccccc34)ccc3ccccc23)c2ccc(-c3ccc(N(c4ccccc4)c4ccccc4)cc3)cc21 ; CC1(C)c2ccccc2N(c2c(-c3cccc4ccccc34)ccc3ccccc23)c2ccc(-c3cccc(N(c4ccccc4)c4ccc(-c5ccccc5)cc4)c3)cc21 ; CC1(C)c2ccccc2-c2ccc(N(c3ccccc3)c3ccc(-c4ccc5c(c4)C(C)(C)c4ccccc4N5c4c(-c5cccc6ccccc56)ccc5ccccc45)cc3)cc21 ; CC1(C)c2ccccc2N(c2c(-c3cccc4ccccc34)ccc3ccccc23)c2ccc(-c3ccc(-c4ccc(N(c5ccccc5)c5ccccc5)cc4)cc3)cc21]

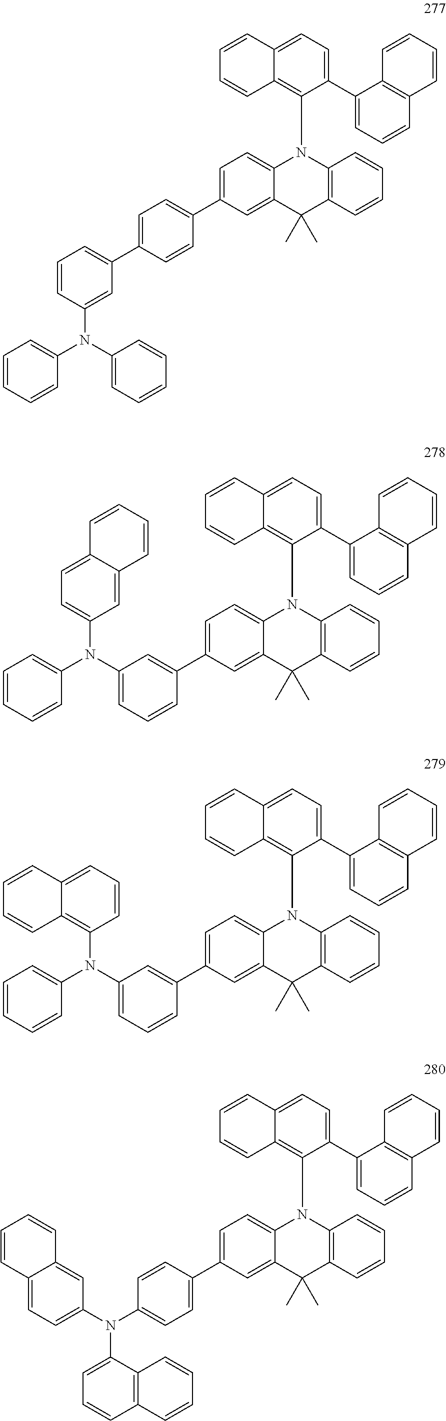 [SMILES: CC1(C)c2ccccc2N(c2c(-c3cccc4ccccc34)ccc3ccccc23)c2ccc(-c3ccc(-c4cccc(N(c5ccccc5)c5ccccc5)c4)cc3)cc21 ; CC1(C)c2ccccc2N(c2c(-c3cccc4ccccc34)ccc3ccccc23)c2ccc(-c3cccc(N(c4ccccc4)c4ccc5ccccc5c4)c3)cc21 ; CC1(C)c2ccccc2N(c2c(-c3cccc4ccccc34)ccc3ccccc23)c2ccc(-c3cccc(N(c4ccccc4)c4cccc5ccccc45)c3)cc21 ; CC1(C)c2ccccc2N(c2c(-c3cccc4ccccc34)ccc3ccccc23)c2ccc(-c3ccc(N(c4ccc5ccccc5c4)c4cccc5ccccc45)cc3)cc21]

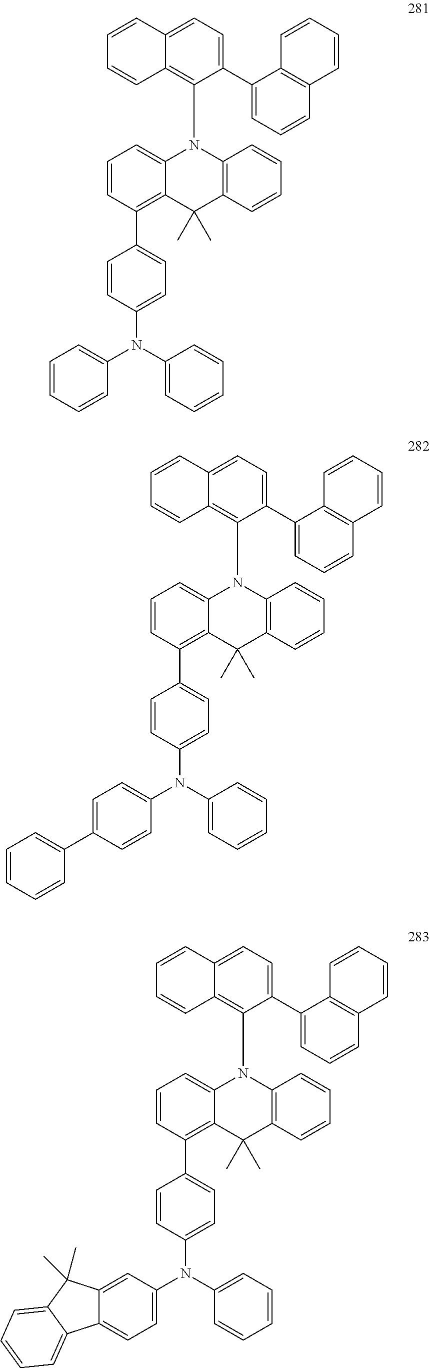

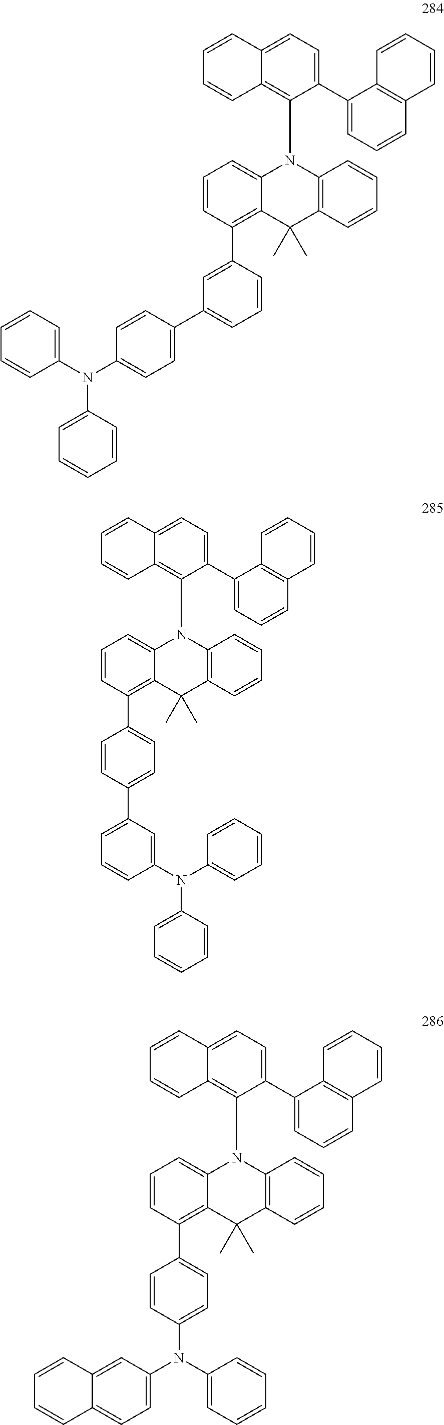 [SMILES: CC1(C)c2ccccc2N(c2c(-c3cccc4ccccc34)ccc3ccccc23)c2cccc(-c3cccc(-c4ccc(N(c5ccccc5)c5ccccc5)cc4)c3)c21 ; CC1(C)c2ccccc2N(c2c(-c3cccc4ccccc34)ccc3ccccc23)c2cccc(-c3ccc(-c4cccc(N(c5ccccc5)c5ccccc5)c4)cc3)c21 ; CC1(C)c2ccccc2N(c2c(-c3cccc4ccccc34)ccc3ccccc23)c2cccc(-c3ccc(N(c4ccccc4)c4ccc5ccccc5c4)cc3)c21]

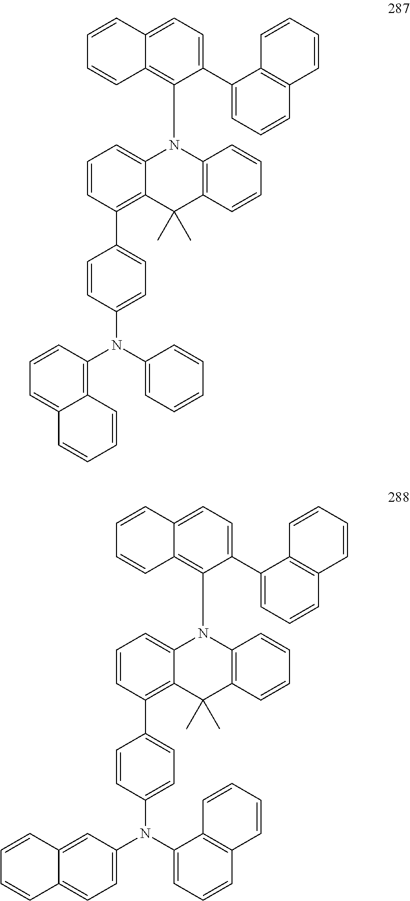 [SMILES: CC1(C)c2ccccc2N(c2c(-c3cccc4ccccc34)ccc3ccccc23)c2cccc(-c3ccc(N(c4ccccc4)c4cccc5ccccc45)cc3)c21 ; CC1(C)c2ccccc2N(c2c(-c3cccc4ccccc34)ccc3ccccc23)c2cccc(-c3ccc(N(c4ccc5ccccc5c4)c4cccc5ccccc45)cc3)c21]

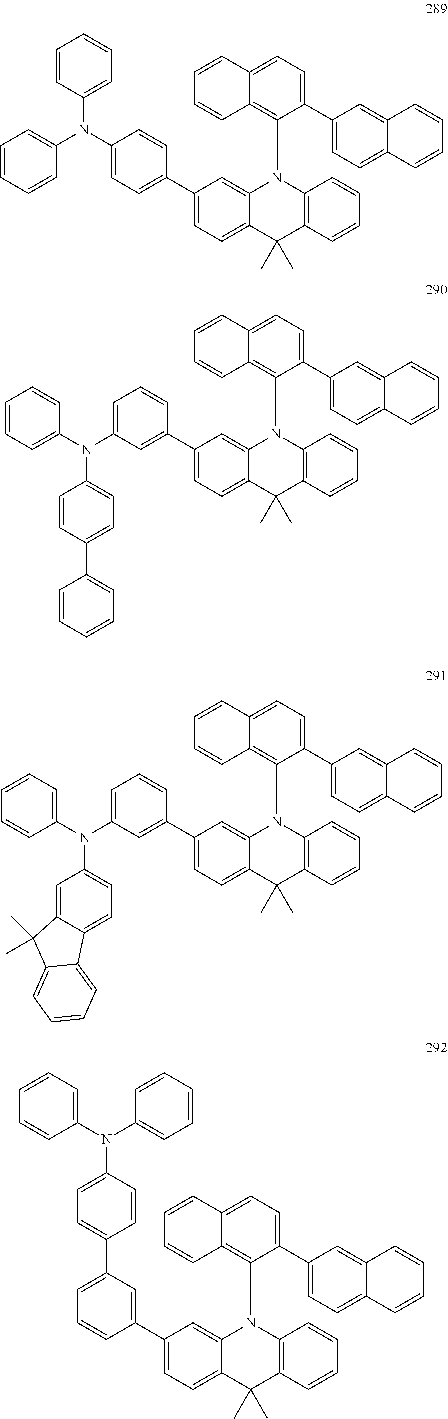 [SMILES: CC1(C)c2ccccc2N(c2c(-c3ccc4ccccc4c3)ccc3ccccc23)c2cc(-c3ccc(N(c4ccccc4)c4ccccc4)cc3)ccc21 ; CC1(C)c2ccccc2N(c2c(-c3ccc4ccccc4c3)ccc3ccccc23)c2cc(-c3cccc(N(c4ccccc4)c4ccc(-c5ccccc5)cc4)c3)ccc21 ; CC1(C)c2ccccc2-c2ccc(N(c3ccccc3)c3cccc(-c4ccc5c(c4)N(c4c(-c6ccc7ccccc7c6)ccc6ccccc46)c4ccccc4C5(C)C)c3)cc21 ; CC1(C)c2ccccc2N(c2c(-c3ccc4ccccc4c3)ccc3ccccc23)c2cc(-c3cccc(-c4ccc(N(c5ccccc5)c5ccccc5)cc4)c3)ccc21]

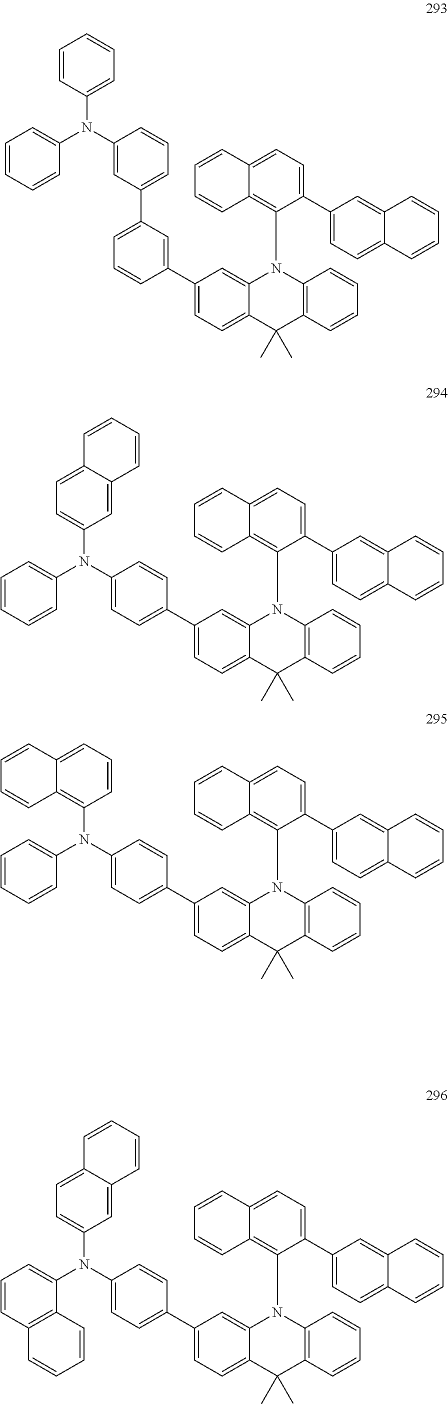 [SMILES: CC1(C)c2ccccc2N(c2c(-c3ccc4ccccc4c3)ccc3ccccc23)c2cc(-c3cccc(-c4cccc(N(c5ccccc5)c5ccccc5)c4)c3)ccc21 ; CC1(C)c2ccccc2N(c2c(-c3ccc4ccccc4c3)ccc3ccccc23)c2cc(-c3ccc(N(c4ccccc4)c4ccc5ccccc5c4)cc3)ccc21 ; CC1(C)c2ccccc2N(c2c(-c3ccc4ccccc4c3)ccc3ccccc23)c2cc(-c3ccc(N(c4ccccc4)c4cccc5ccccc45)cc3)ccc21 ; CC1(C)c2ccccc2N(c2c(-c3ccc4ccccc4c3)ccc3ccccc23)c2cc(-c3ccc(N(c4ccc5ccccc5c4)c4cccc5ccccc45)cc3)ccc21]

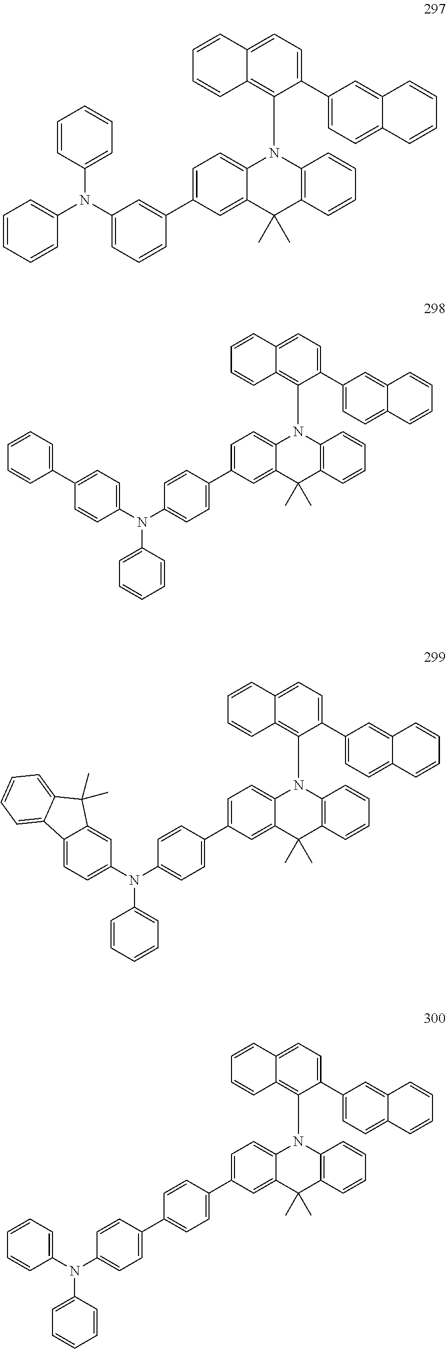 [SMILES: CC1(C)c2ccccc2N(c2c(-c3ccc4ccccc4c3)ccc3ccccc23)c2ccc(-c3cccc(N(c4ccccc4)c4ccccc4)c3)cc21 ; CC1(C)c2ccccc2N(c2c(-c3ccc4ccccc4c3)ccc3ccccc23)c2ccc(-c3ccc(N(c4ccccc4)c4ccc(-c5ccccc5)cc4)cc3)cc21 ; CC1(C)c2ccccc2-c2ccc(N(c3ccccc3)c3ccc(-c4ccc5c(c4)C(C)(C)c4ccccc4N5c4c(-c5ccc6ccccc6c5)ccc5ccccc45)cc3)cc21 ; CC1(C)c2ccccc2N(c2c(-c3ccc4ccccc4c3)ccc3ccccc23)c2ccc(-c3ccc(-c4ccc(N(c5ccccc5)c5ccccc5)cc4)cc3)cc21]

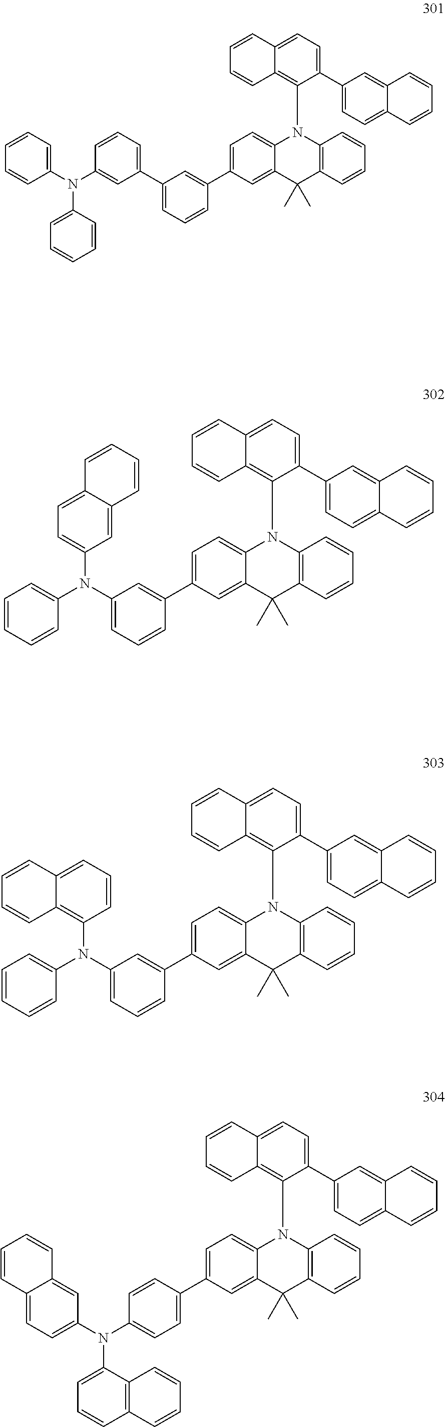 [SMILES: CC1(C)c2ccccc2N(c2c(-c3ccc4ccccc4c3)ccc3ccccc23)c2ccc(-c3cccc(-c4cccc(N(c5ccccc5)c5ccccc5)c4)c3)cc21 ; CC1(C)c2ccccc2N(c2c(-c3ccc4ccccc4c3)ccc3ccccc23)c2ccc(-c3cccc(N(c4ccccc4)c4ccc5ccccc5c4)c3)cc21 ; CC1(C)c2ccccc2N(c2c(-c3ccc4ccccc4c3)ccc3ccccc23)c2ccc(-c3cccc(N(c4ccccc4)c4cccc5ccccc45)c3)cc21 ; CC1(C)c2ccccc2N(c2c(-c3ccc4ccccc4c3)ccc3ccccc23)c2ccc(-c3ccc(N(c4ccc5ccccc5c4)c4cccc5ccccc45)cc3)cc21]

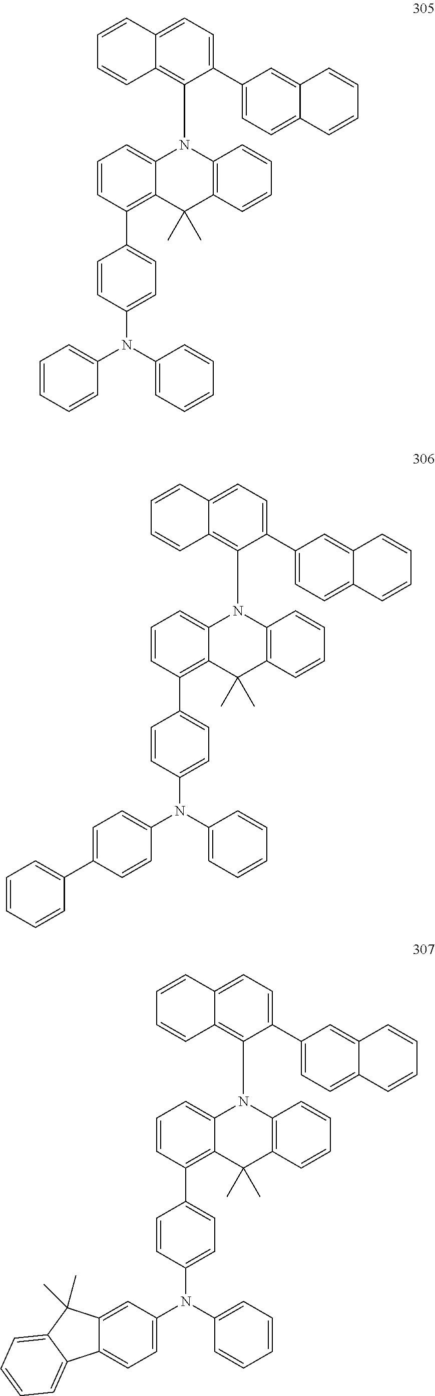 [SMILES: CC1(C)c2ccccc2N(c2c(-c3ccc4ccccc4c3)ccc3ccccc23)c2cccc(-c3ccc(N(c4ccccc4)c4ccccc4)cc3)c21 ; CC1(C)c2ccccc2N(c2c(-c3ccc4ccccc4c3)ccc3ccccc23)c2cccc(-c3ccc(N(c4ccccc4)c4ccc(-c5ccccc5)cc4)cc3)c21 ; CC1(C)c2ccccc2-c2ccc(N(c3ccccc3)c3ccc(-c4cccc5c4C(C)(C)c4ccccc4N5c4c(-c5ccc6ccccc6c5)ccc5ccccc45)cc3)cc21]

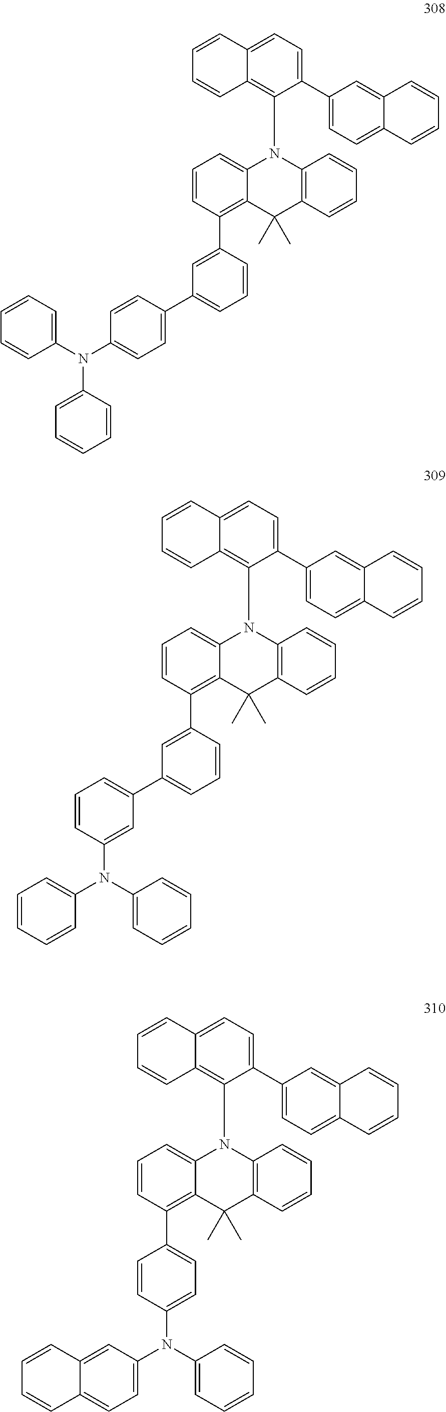 [SMILES: CC1(C)c2ccccc2N(c2c(-c3ccc4ccccc4c3)ccc3ccccc23)c2cccc(-c3cccc(-c4ccc(N(c5ccccc5)c5ccccc5)cc4)c3)c21 ; CC1(C)c2ccccc2N(c2c(-c3ccc4ccccc4c3)ccc3ccccc23)c2cccc(-c3cccc(-c4cccc(N(c5ccccc5)c5ccccc5)c4)c3)c21 ; CC1(C)c2ccccc2N(c2c(-c3ccc4ccccc4c3)ccc3ccccc23)c2cccc(-c3ccc(N(c4ccccc4)c4ccc5ccccc5c4)cc3)c21]

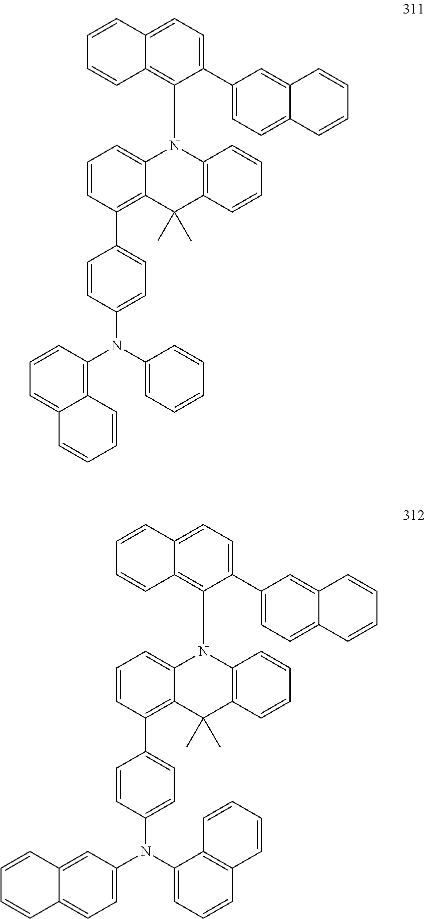 [SMILES: CC1(C)c2ccccc2N(c2c(-c3ccc4ccccc4c3)ccc3ccccc23)c2cccc(-c3ccc(N(c4ccccc4)c4cccc5ccccc45)cc3)c21 ; CC1(C)c2ccccc2N(c2c(-c3ccc4ccccc4c3)ccc3ccccc23)c2cccc(-c3ccc(N(c4ccc5ccccc5c4)c4cccc5ccccc45)cc3)c21]

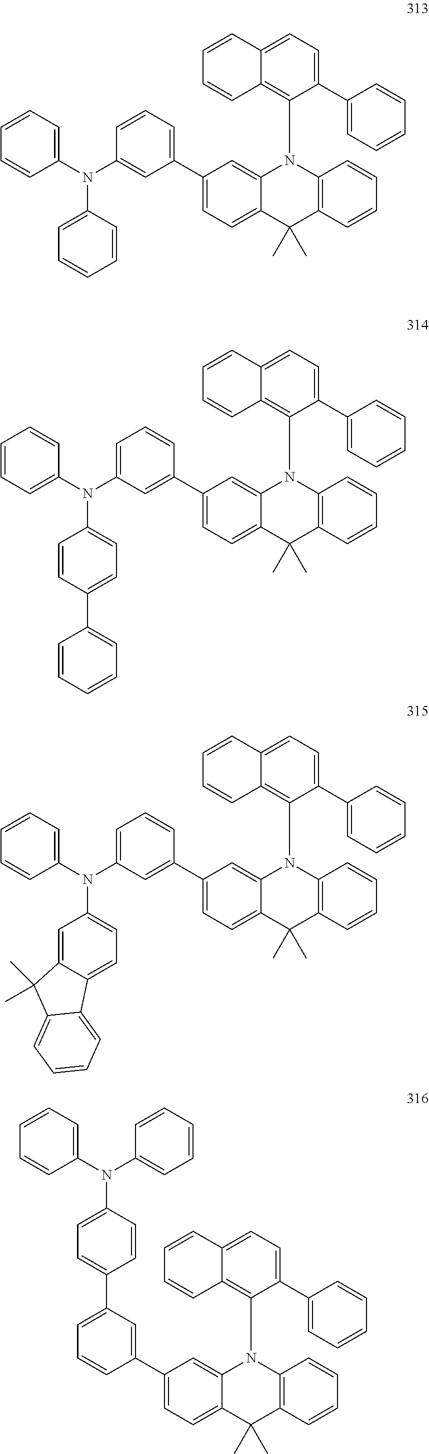

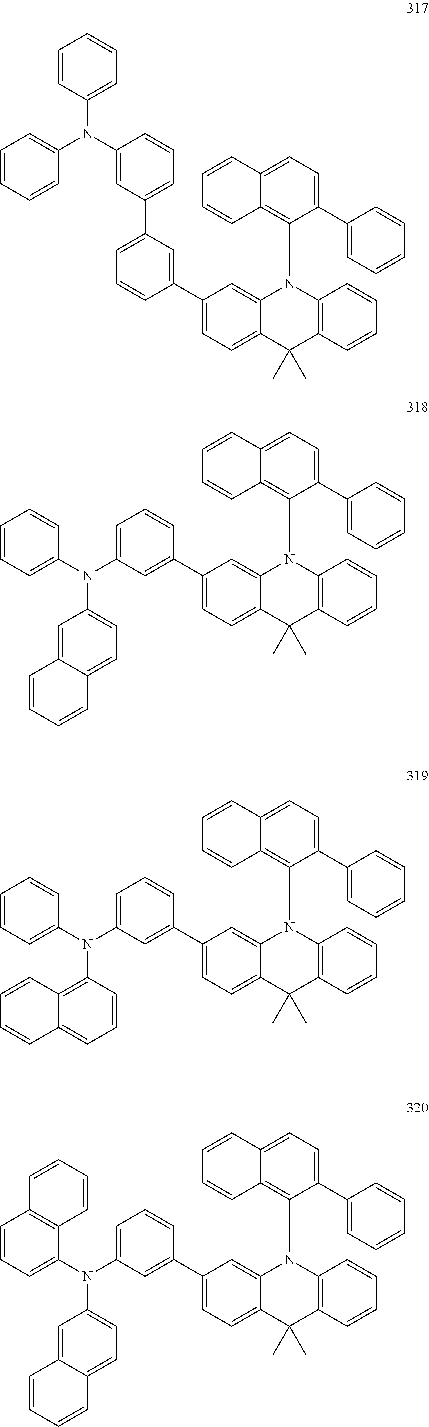 [SMILES: CC1(C)c2ccccc2N(c2c(-c3ccccc3)ccc3ccccc23)c2cc(-c3cccc(-c4cccc(N(c5ccccc5)c5ccccc5)c4)c3)ccc21 ; CC1(C)c2ccccc2N(c2c(-c3ccccc3)ccc3ccccc23)c2cc(-c3cccc(N(c4ccccc4)c4ccc5ccccc5c4)c3)ccc21 ; CC1(C)c2ccccc2N(c2c(-c3ccccc3)ccc3ccccc23)c2cc(-c3cccc(N(c4ccccc4)c4cccc5ccccc45)c3)ccc21 ; CC1(C)c2ccccc2N(c2c(-c3ccccc3)ccc3ccccc23)c2cc(-c3cccc(N(c4ccc5ccccc5c4)c4cccc5ccccc45)c3)ccc21]

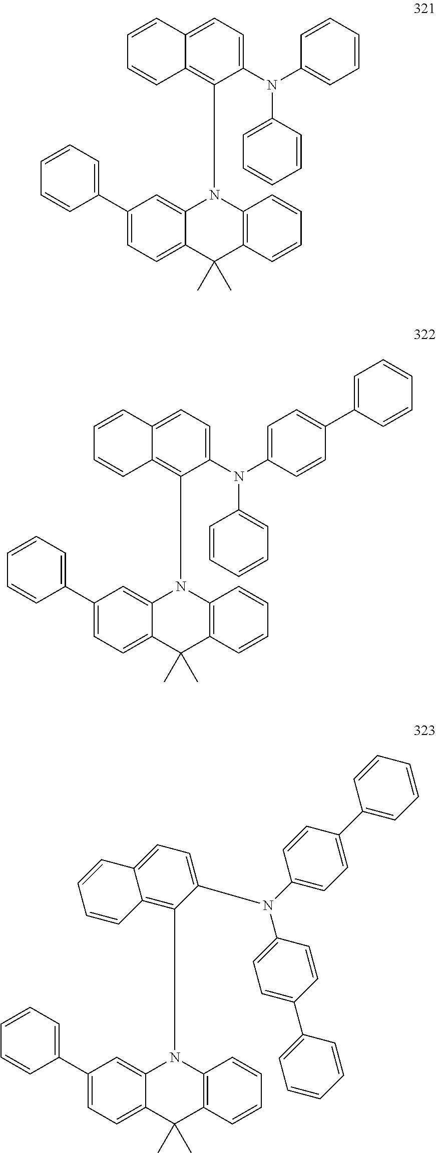 [SMILES: CC1(C)c2ccccc2N(c2c(N(c3ccccc3)c3ccccc3)ccc3ccccc23)c2cc(-c3ccccc3)ccc21 ; CC1(C)c2ccccc2N(c2c(N(c3ccccc3)c3ccc(-c4ccccc4)cc3)ccc3ccccc23)c2cc(-c3ccccc3)ccc21 ; CC1(C)c2ccccc2N(c2c(N(c3ccc(-c4ccccc4)cc3)c3ccc(-c4ccccc4)cc3)ccc3ccccc23)c2cc(-c3ccccc3)ccc21]

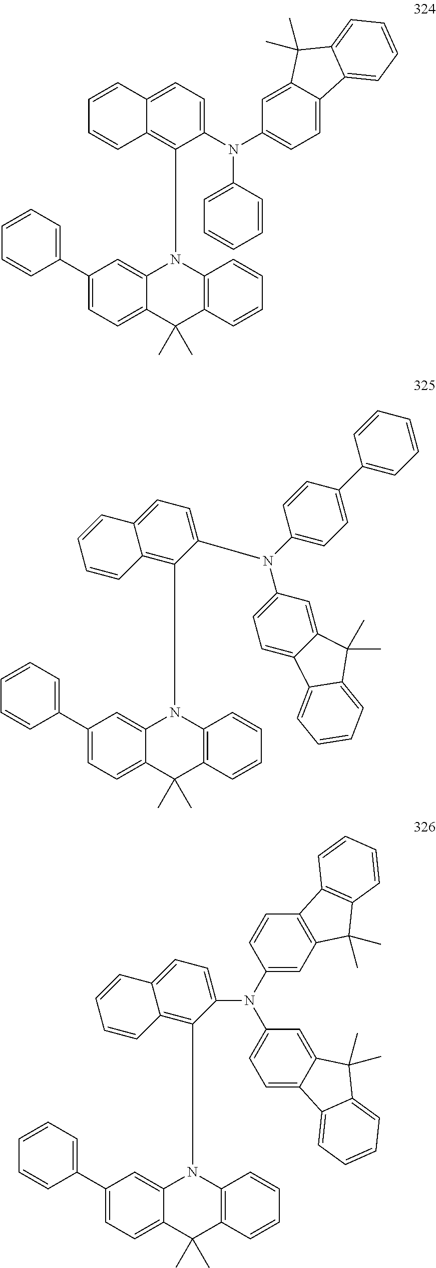 [SMILES: CC1(C)c2ccccc2-c2ccc(N(c3ccccc3)c3ccc4ccccc4c3N3c4ccccc4C(C)(C)c4ccc(-c5ccccc5)cc43)cc21 ; CC1(C)c2ccccc2-c2ccc(N(c3ccc(-c4ccccc4)cc3)c3ccc4ccccc4c3N3c4ccccc4C(C)(C)c4ccc(-c5ccccc5)cc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4ccc(-c5ccccc5)cc43)cc21]

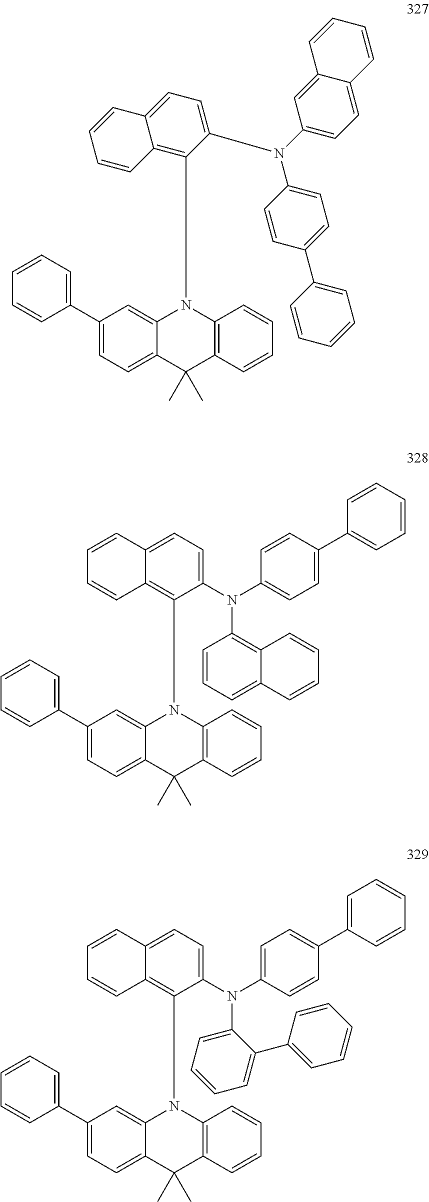 [SMILES: CC1(C)c2ccccc2N(c2c(N(c3ccc(-c4ccccc4)cc3)c3ccc4ccccc4c3)ccc3ccccc23)c2cc(-c3ccccc3)ccc21 ; CC1(C)c2ccccc2N(c2c(N(c3ccc(-c4ccccc4)cc3)c3cccc4ccccc34)ccc3ccccc23)c2cc(-c3ccccc3)ccc21 ; CC1(C)c2ccccc2N(c2c(N(c3ccc(-c4ccccc4)cc3)c3ccccc3-c3ccccc3)ccc3ccccc23)c2cc(-c3ccccc3)ccc21]

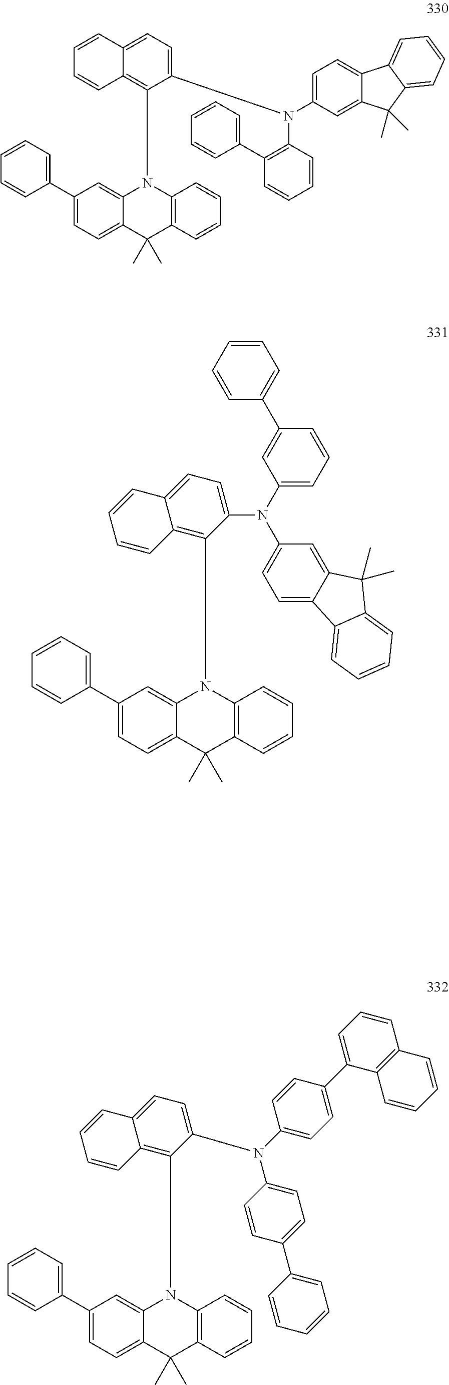 [SMILES: CC1(C)c2ccccc2-c2ccc(N(c3ccccc3-c3ccccc3)c3ccc4ccccc4c3N3c4ccccc4C(C)(C)c4ccc(-c5ccccc5)cc43)cc21 ; CC1(C)c2ccccc2-c2ccc(N(c3cccc(-c4ccccc4)c3)c3ccc4ccccc4c3N3c4ccccc4C(C)(C)c4ccc(-c5ccccc5)cc43)cc21 ; CC1(C)c2ccccc2N(c2c(N(c3ccc(-c4ccccc4)cc3)c3ccc(-c4cccc5ccccc45)cc3)ccc3ccccc23)c2cc(-c3ccccc3)ccc21]

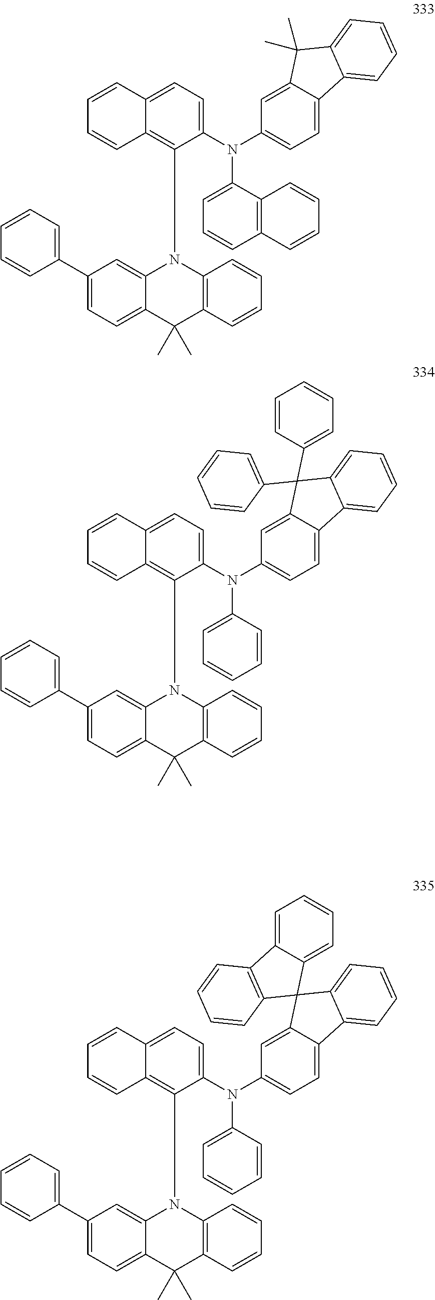 [SMILES: CC1(C)c2ccccc2-c2ccc(N(c3ccc4ccccc4c3N3c4ccccc4C(C)(C)c4ccc(-c5ccccc5)cc43)c3cccc4ccccc34)cc21 ; CC1(C)c2ccccc2N(c2c(N(c3ccccc3)c3ccc4c(c3)C(c3ccccc3)(c3ccccc3)c3ccccc3-4)ccc3ccccc23)c2cc(-c3ccccc3)ccc21 ; CC1(C)c2ccccc2N(c2c(N(c3ccccc3)c3ccc4c(c3)C3(c5ccccc5-c5ccccc53)c3ccccc3-4)ccc3ccccc23)c2cc(-c3ccccc3)ccc21]

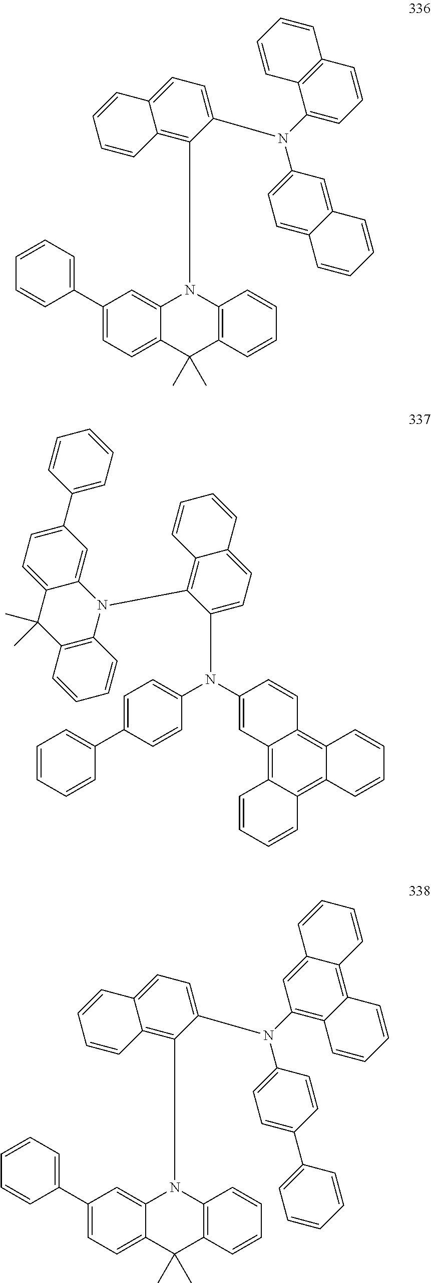 [SMILES: CC1(C)c2ccccc2N(c2c(N(c3ccc4ccccc4c3)c3cccc4ccccc34)ccc3ccccc23)c2cc(-c3ccccc3)ccc21 ; CC1(C)c2ccccc2N(c2c(N(c3ccc(-c4ccccc4)cc3)c3ccc4c5ccccc5c5ccccc5c4c3)ccc3ccccc23)c2cc(-c3ccccc3)ccc21 ; CC1(C)c2ccccc2N(c2c(N(c3ccc(-c4ccccc4)cc3)c3cc4ccccc4c4ccccc34)ccc3ccccc23)c2cc(-c3ccccc3)ccc21]

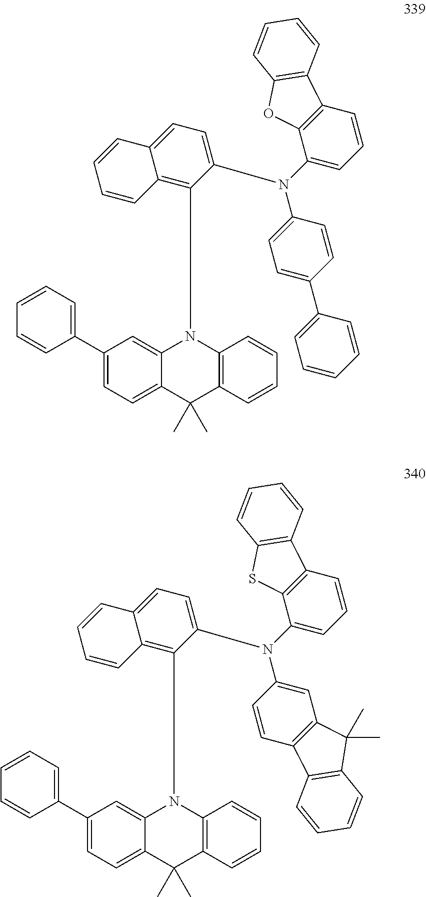 [SMILES: CC1(C)c2ccccc2N(c2c(N(c3ccc(-c4ccccc4)cc3)c3cccc4c3oc3ccccc34)ccc3ccccc23)c2cc(-c3ccccc3)ccc21 ; CC1(C)c2ccccc2-c2ccc(N(c3ccc4ccccc4c3N3c4ccccc4C(C)(C)c4ccc(-c5ccccc5)cc43)c3cccc4c3sc3ccccc34)cc21]

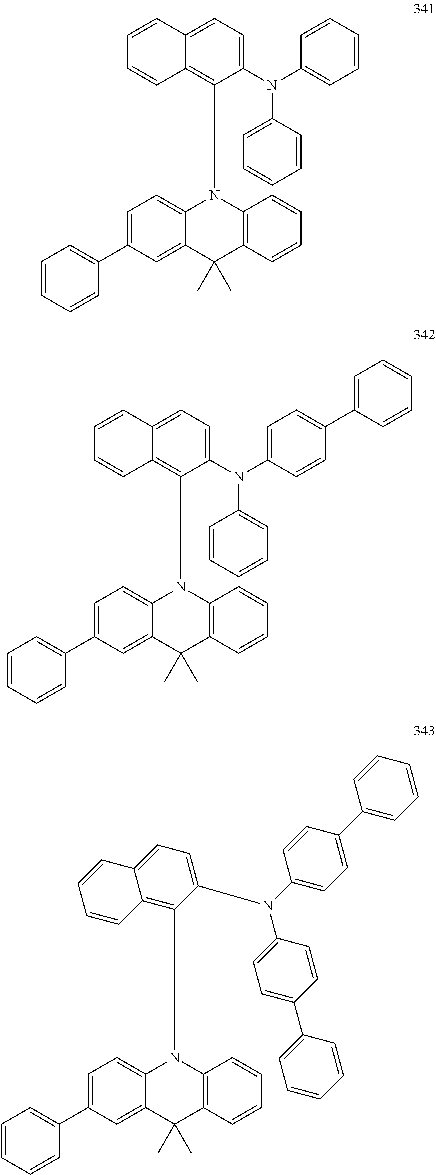 [SMILES: CC1(C)c2ccccc2N(c2c(N(c3ccccc3)c3ccccc3)ccc3ccccc23)c2ccc(-c3ccccc3)cc21 ; CC1(C)c2ccccc2N(c2c(N(c3ccccc3)c3ccc(-c4ccccc4)cc3)ccc3ccccc23)c2ccc(-c3ccccc3)cc21 ; CC1(C)c2ccccc2N(c2c(N(c3ccc(-c4ccccc4)cc3)c3ccc(-c4ccccc4)cc3)ccc3ccccc23)c2ccc(-c3ccccc3)cc21]

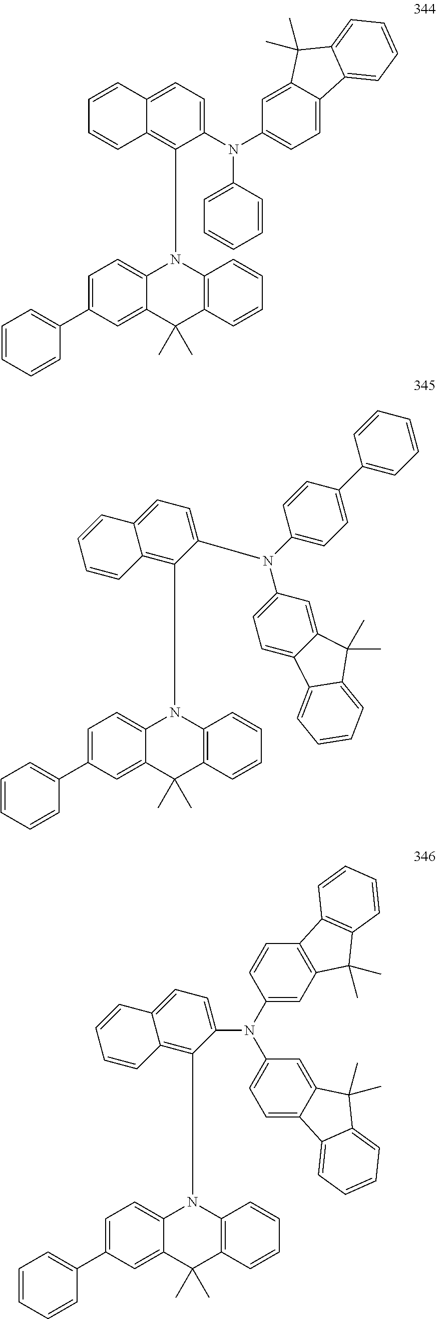 [SMILES: CC1(C)c2ccccc2-c2ccc(N(c3ccccc3)c3ccc4ccccc4c3N3c4ccccc4C(C)(C)c4cc(-c5ccccc5)ccc43)cc21 ; CC1(C)c2ccccc2-c2ccc(N(c3ccc(-c4ccccc4)cc3)c3ccc4ccccc4c3N3c4ccccc4C(C)(C)c4cc(-c5ccccc5)ccc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4cc(-c5ccccc5)ccc43)cc21]

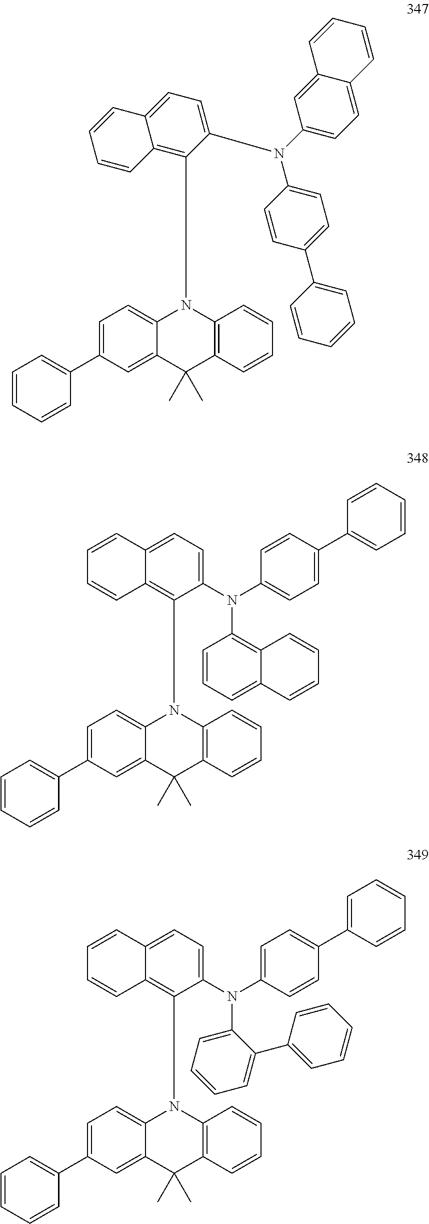 [SMILES: CC1(C)c2ccccc2N(c2c(N(c3ccc(-c4ccccc4)cc3)c3ccc4ccccc4c3)ccc3ccccc23)c2ccc(-c3ccccc3)cc21 ; CC1(C)c2ccccc2N(c2c(N(c3ccc(-c4ccccc4)cc3)c3cccc4ccccc34)ccc3ccccc23)c2ccc(-c3ccccc3)cc21 ; CC1(C)c2ccccc2N(c2c(N(c3ccc(-c4ccccc4)cc3)c3ccccc3-c3ccccc3)ccc3ccccc23)c2ccc(-c3ccccc3)cc21]

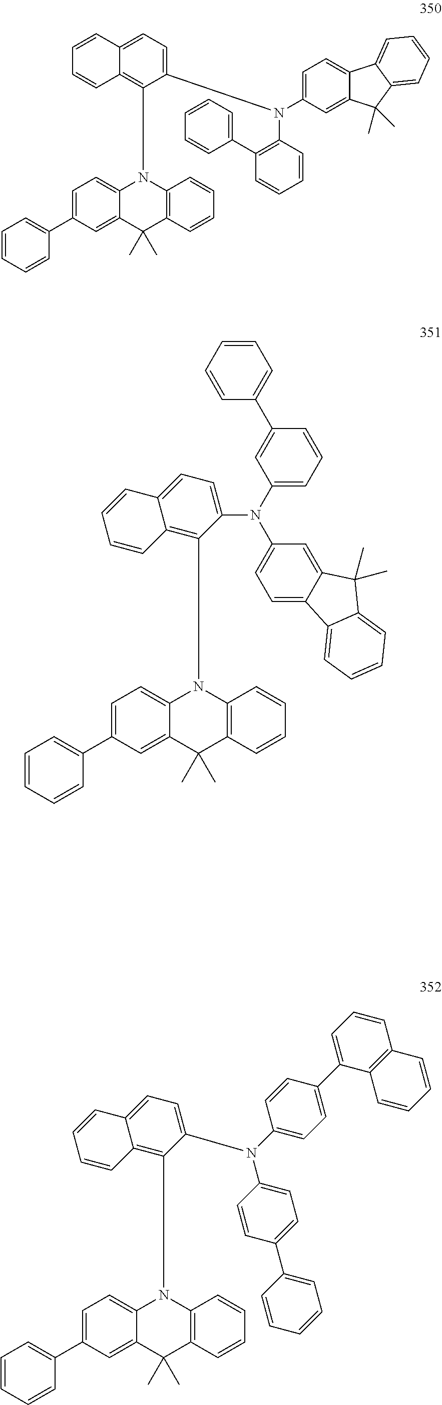 [SMILES: CC1(C)c2ccccc2-c2ccc(N(c3ccccc3-c3ccccc3)c3ccc4ccccc4c3N3c4ccccc4C(C)(C)c4cc(-c5ccccc5)ccc43)cc21 ; CC1(C)c2ccccc2-c2ccc(N(c3cccc(-c4ccccc4)c3)c3ccc4ccccc4c3N3c4ccccc4C(C)(C)c4cc(-c5ccccc5)ccc43)cc21 ; CC1(C)c2ccccc2N(c2c(N(c3ccc(-c4ccccc4)cc3)c3ccc(-c4cccc5ccccc45)cc3)ccc3ccccc23)c2ccc(-c3ccccc3)cc21]

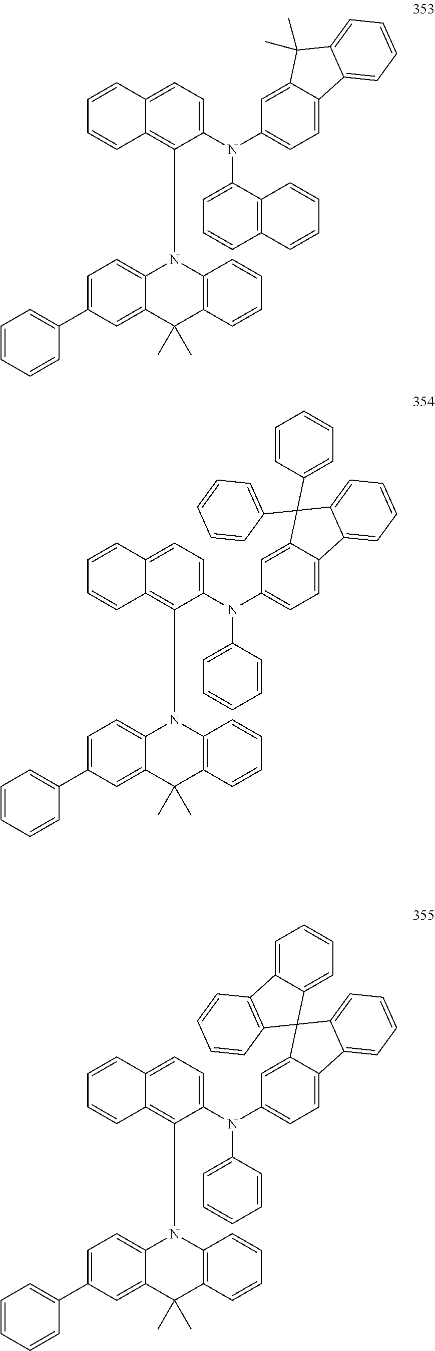 [SMILES: CC1(C)c2ccccc2-c2ccc(N(c3ccc4ccccc4c3N3c4ccccc4C(C)(C)c4cc(-c5ccccc5)ccc43)c3cccc4ccccc34)cc21 ; CC1(C)c2ccccc2N(c2c(N(c3ccccc3)c3ccc4c(c3)C(c3ccccc3)(c3ccccc3)c3ccccc3-4)ccc3ccccc23)c2ccc(-c3ccccc3)cc21 ; CC1(C)c2ccccc2N(c2c(N(c3ccccc3)c3ccc4c(c3)C3(c5ccccc5-c5ccccc53)c3ccccc3-4)ccc3ccccc23)c2ccc(-c3ccccc3)cc21]

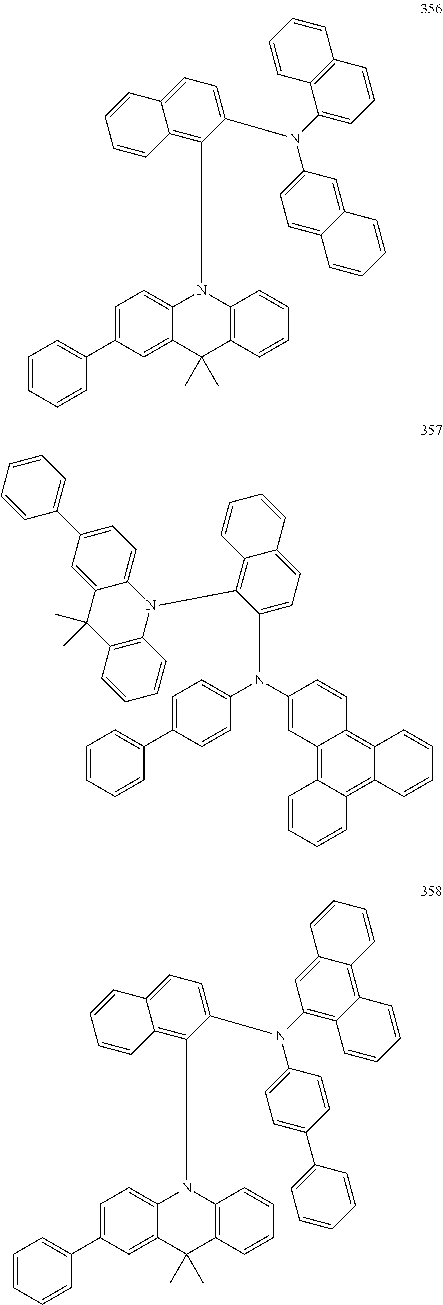 [SMILES: CC1(C)c2ccccc2N(c2c(N(c3ccc4ccccc4c3)c3cccc4ccccc34)ccc3ccccc23)c2ccc(-c3ccccc3)cc21 ; CC1(C)c2ccccc2N(c2c(N(c3ccc(-c4ccccc4)cc3)c3ccc4c5ccccc5c5ccccc5c4c3)ccc3ccccc23)c2ccc(-c3ccccc3)cc21 ; CC1(C)c2ccccc2N(c2c(N(c3ccc(-c4ccccc4)cc3)c3cc4ccccc4c4ccccc34)ccc3ccccc23)c2ccc(-c3ccccc3)cc21]

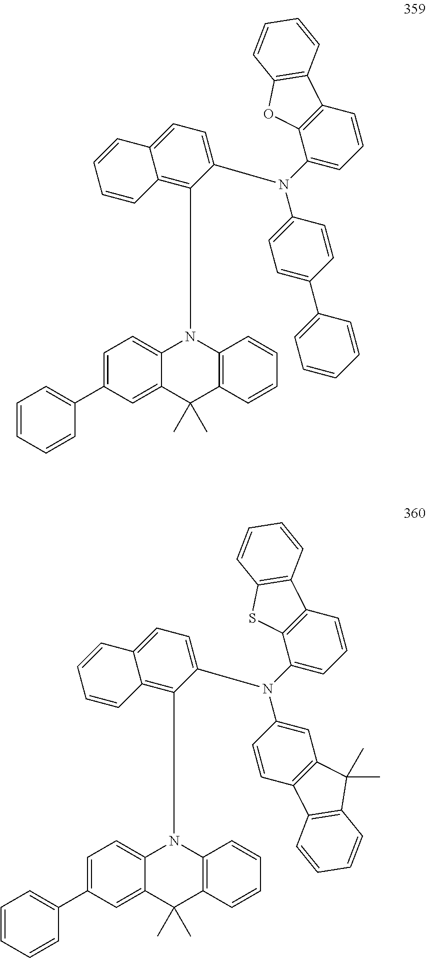 [SMILES: CC1(C)c2ccccc2N(c2c(N(c3ccc(-c4ccccc4)cc3)c3cccc4c3oc3ccccc34)ccc3ccccc23)c2ccc(-c3ccccc3)cc21 ; CC1(C)c2ccccc2-c2ccc(N(c3ccc4ccccc4c3N3c4ccccc4C(C)(C)c4cc(-c5ccccc5)ccc43)c3cccc4c3sc3ccccc34)cc21]

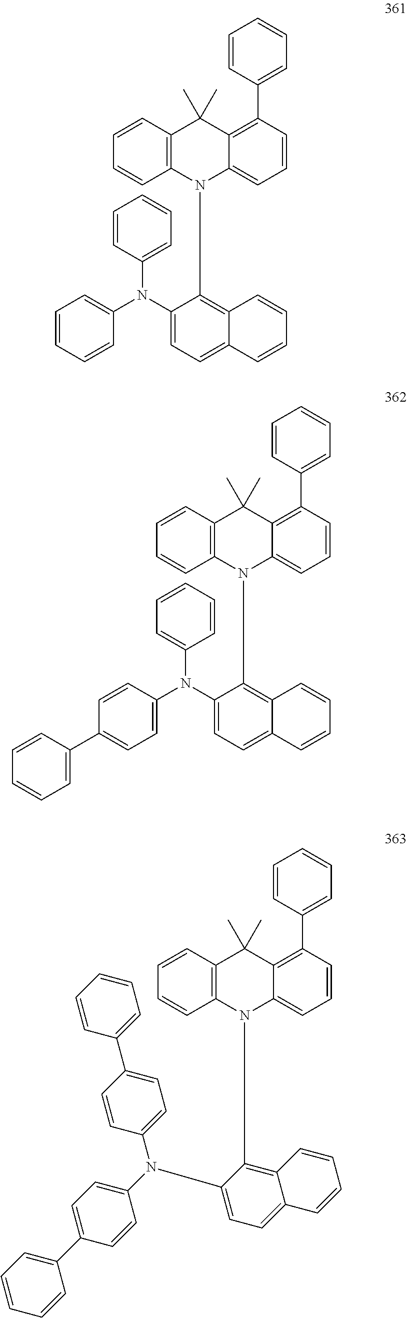 [SMILES: CC1(C)c2ccccc2N(c2c(N(c3ccccc3)c3ccccc3)ccc3ccccc23)c2cccc(-c3ccccc3)c21 ; CC1(C)c2ccccc2N(c2c(N(c3ccccc3)c3ccc(-c4ccccc4)cc3)ccc3ccccc23)c2cccc(-c3ccccc3)c21 ; CC1(C)c2ccccc2N(c2c(N(c3ccc(-c4ccccc4)cc3)c3ccc(-c4ccccc4)cc3)ccc3ccccc23)c2cccc(-c3ccccc3)c21]

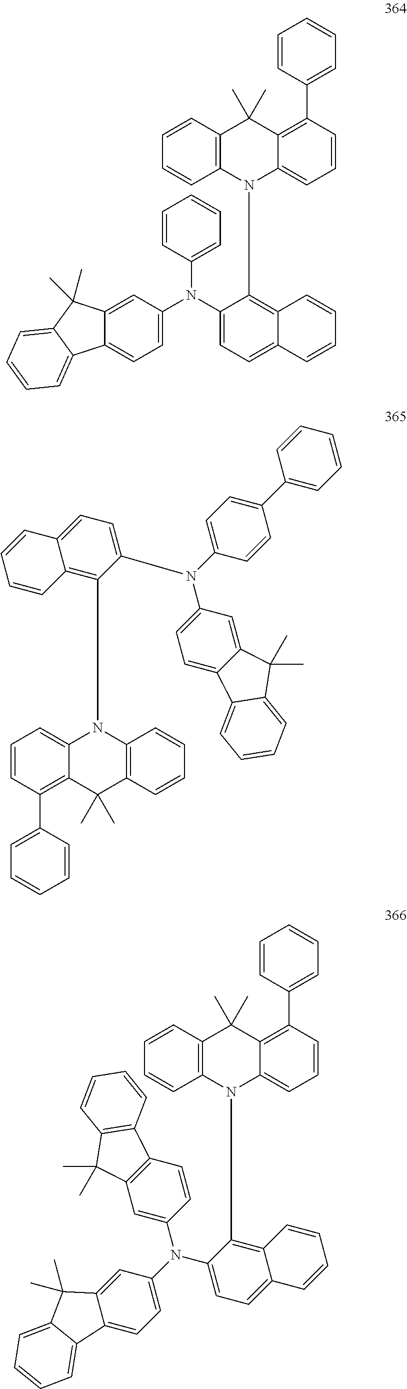 [SMILES: CC1(C)c2ccccc2-c2ccc(N(c3ccccc3)c3ccc4ccccc4c3N3c4ccccc4C(C)(C)c4c(-c5ccccc5)cccc43)cc21 ; CC1(C)c2ccccc2-c2ccc(N(c3ccc(-c4ccccc4)cc3)c3ccc4ccccc4c3N3c4ccccc4C(C)(C)c4c(-c5ccccc5)cccc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4c(-c5ccccc5)cccc43)cc21]

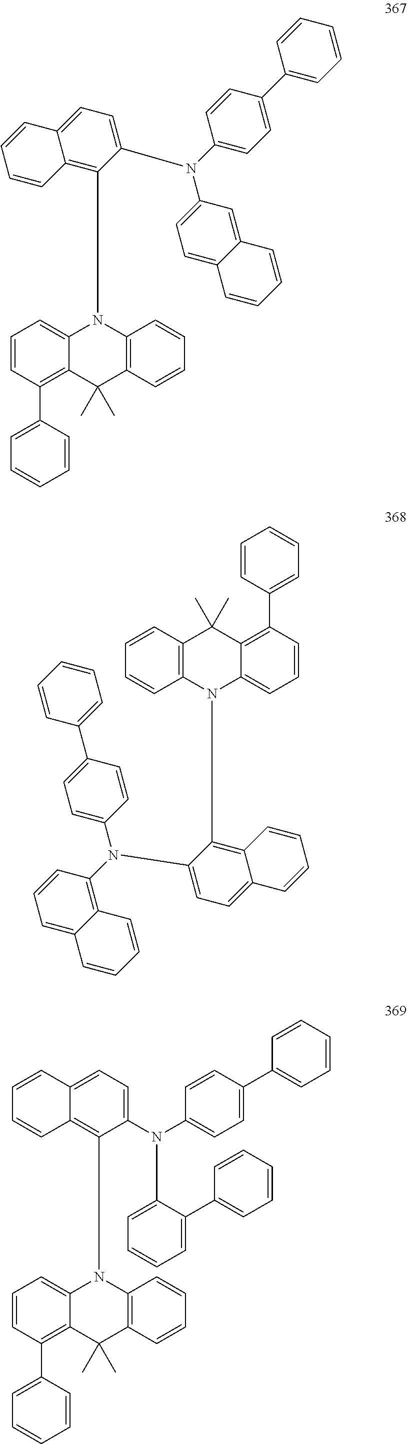 [SMILES: CC1(C)c2ccccc2N(c2c(N(c3ccc(-c4ccccc4)cc3)c3ccc4ccccc4c3)ccc3ccccc23)c2cccc(-c3ccccc3)c21 ; CC1(C)c2ccccc2N(c2c(N(c3ccc(-c4ccccc4)cc3)c3cccc4ccccc34)ccc3ccccc23)c2cccc(-c3ccccc3)c21 ; CC1(C)c2ccccc2N(c2c(N(c3ccc(-c4ccccc4)cc3)c3ccccc3-c3ccccc3)ccc3ccccc23)c2cccc(-c3ccccc3)c21]

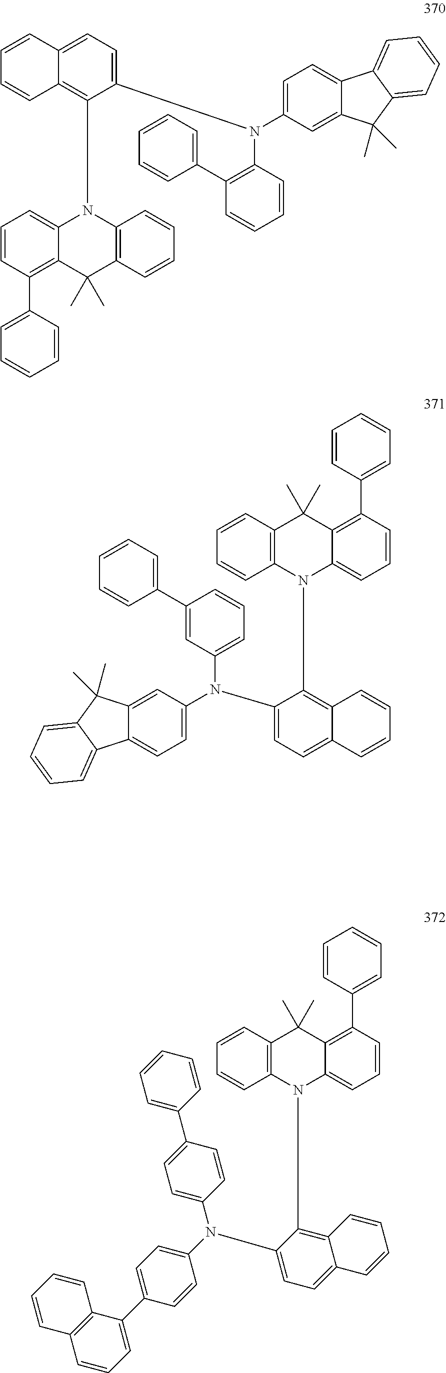 [SMILES: CC1(C)c2ccccc2-c2ccc(N(c3ccccc3-c3ccccc3)c3ccc4ccccc4c3N3c4ccccc4C(C)(C)c4c(-c5ccccc5)cccc43)cc21 ; CC1(C)c2ccccc2-c2ccc(N(c3cccc(-c4ccccc4)c3)c3ccc4ccccc4c3N3c4ccccc4C(C)(C)c4c(-c5ccccc5)cccc43)cc21 ; CC1(C)c2ccccc2N(c2c(N(c3ccc(-c4ccccc4)cc3)c3ccc(-c4cccc5ccccc45)cc3)ccc3ccccc23)c2cccc(-c3ccccc3)c21]

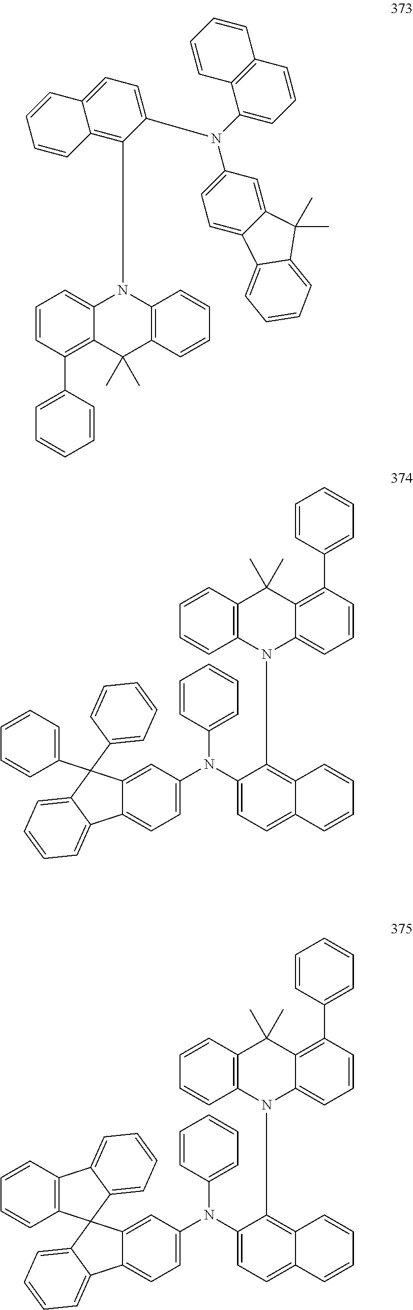 [SMILES: CC1(C)c2ccccc2-c2ccc(N(c3ccc4ccccc4c3N3c4ccccc4C(C)(C)c4c(-c5ccccc5)cccc43)c3cccc4ccccc34)cc21 ; CC1(C)c2ccccc2N(c2c(N(c3ccccc3)c3ccc4c(c3)C(c3ccccc3)(c3ccccc3)c3ccccc3-4)ccc3ccccc23)c2cccc(-c3ccccc3)c21 ; CC1(C)c2ccccc2N(c2c(N(c3ccccc3)c3ccc4c(c3)C3(c5ccccc5-c5ccccc53)c3ccccc3-4)ccc3ccccc23)c2cccc(-c3ccccc3)c21]

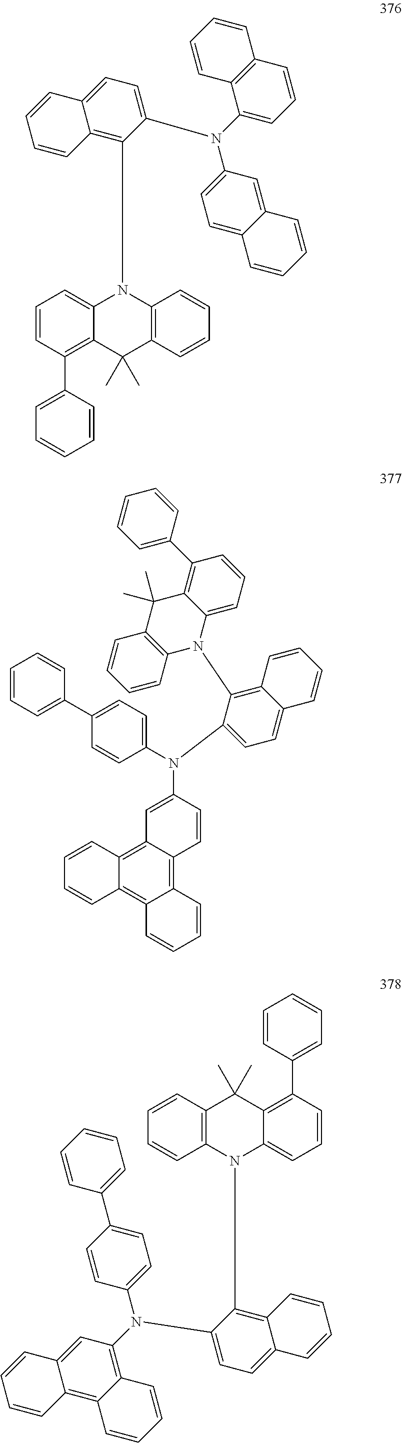 [SMILES: CC1(C)c2ccccc2N(c2c(N(c3ccc4ccccc4c3)c3cccc4ccccc34)ccc3ccccc23)c2cccc(-c3ccccc3)c21 ; CC1(C)c2ccccc2N(c2c(N(c3ccc(-c4ccccc4)cc3)c3ccc4c5ccccc5c5ccccc5c4c3)ccc3ccccc23)c2cccc(-c3ccccc3)c21 ; CC1(C)c2ccccc2N(c2c(N(c3ccc(-c4ccccc4)cc3)c3cc4ccccc4c4ccccc34)ccc3ccccc23)c2cccc(-c3ccccc3)c21]

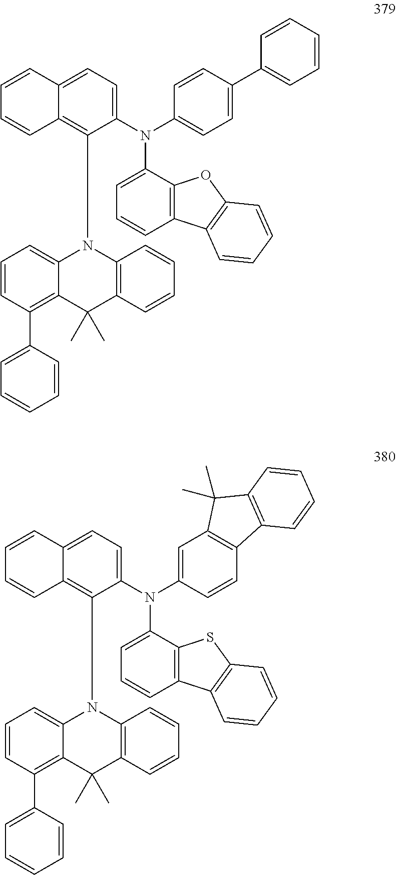 [SMILES: CC1(C)c2ccccc2N(c2c(N(c3ccc(-c4ccccc4)cc3)c3cccc4c3oc3ccccc34)ccc3ccccc23)c2cccc(-c3ccccc3)c21 ; CC1(C)c2ccccc2-c2ccc(N(c3ccc4ccccc4c3N3c4ccccc4C(C)(C)c4c(-c5ccccc5)cccc43)c3cccc4c3sc3ccccc34)cc21]

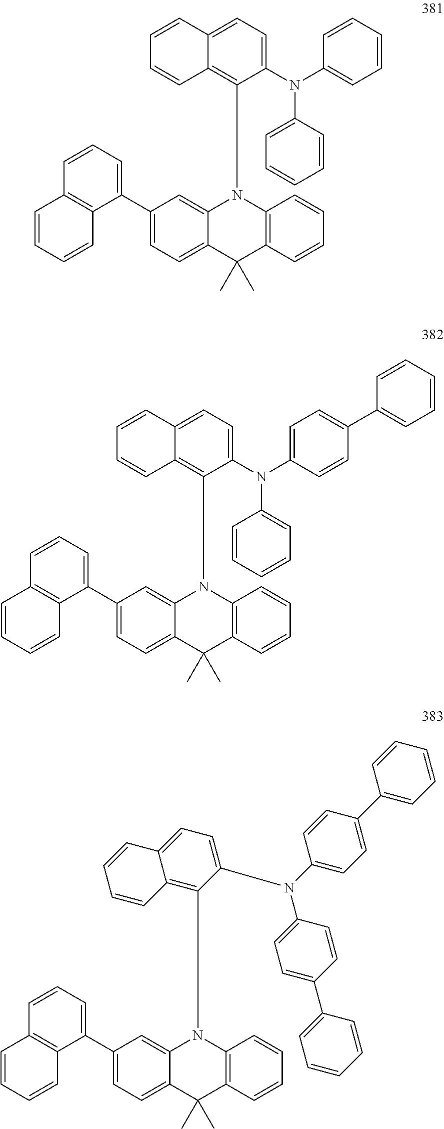 [SMILES: CC1(C)c2ccccc2N(c2c(N(c3ccccc3)c3ccccc3)ccc3ccccc23)c2cc(-c3cccc4ccccc34)ccc21 ; CC1(C)c2ccccc2N(c2c(N(c3ccccc3)c3ccc(-c4ccccc4)cc3)ccc3ccccc23)c2cc(-c3cccc4ccccc34)ccc21 ; CC1(C)c2ccccc2N(c2c(N(c3ccc(-c4ccccc4)cc3)c3ccc(-c4ccccc4)cc3)ccc3ccccc23)c2cc(-c3cccc4ccccc34)ccc21]

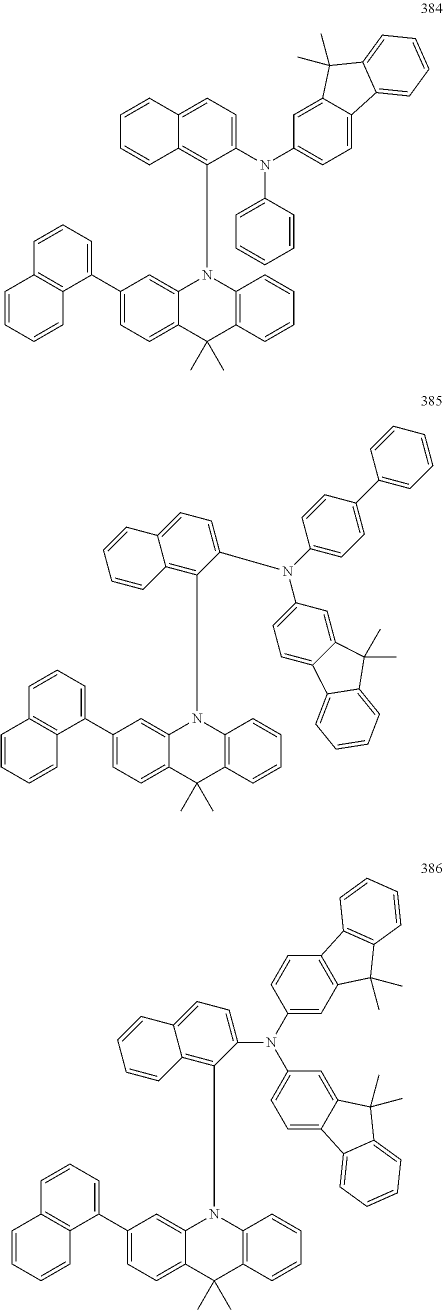 [SMILES: CC1(C)c2ccccc2-c2ccc(N(c3ccccc3)c3ccc4ccccc4c3N3c4ccccc4C(C)(C)c4ccc(-c5cccc6ccccc56)cc43)cc21 ; CC1(C)c2ccccc2-c2ccc(N(c3ccc(-c4ccccc4)cc3)c3ccc4ccccc4c3N3c4ccccc4C(C)(C)c4ccc(-c5cccc6ccccc56)cc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4ccc(-c5cccc6ccccc56)cc43)cc21]

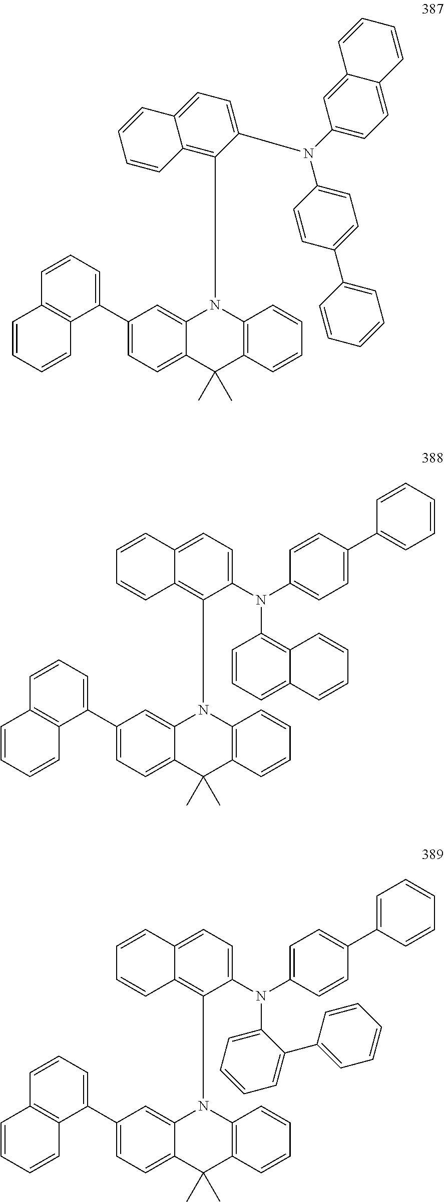 [SMILES: CC1(C)c2ccccc2N(c2c(N(c3ccc(-c4ccccc4)cc3)c3ccc4ccccc4c3)ccc3ccccc23)c2cc(-c3cccc4ccccc34)ccc21 ; CC1(C)c2ccccc2N(c2c(N(c3ccc(-c4ccccc4)cc3)c3cccc4ccccc34)ccc3ccccc23)c2cc(-c3cccc4ccccc34)ccc21 ; CC1(C)c2ccccc2N(c2c(N(c3ccc(-c4ccccc4)cc3)c3ccccc3-c3ccccc3)ccc3ccccc23)c2cc(-c3cccc4ccccc34)ccc21]

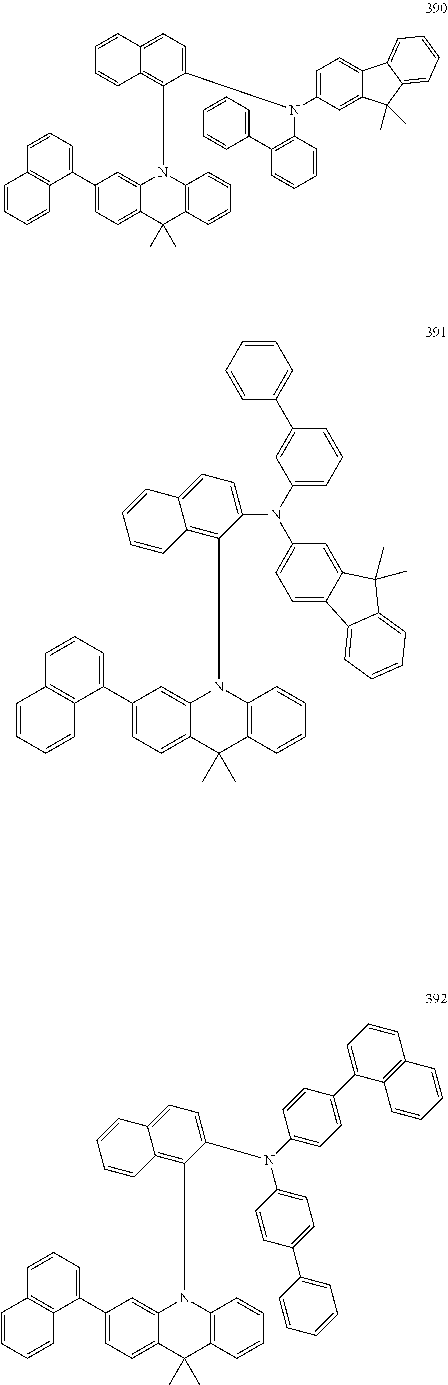 [SMILES: CC1(C)c2ccccc2-c2ccc(N(c3ccccc3-c3ccccc3)c3ccc4ccccc4c3N3c4ccccc4C(C)(C)c4ccc(-c5cccc6ccccc56)cc43)cc21 ; CC1(C)c2ccccc2-c2ccc(N(c3cccc(-c4ccccc4)c3)c3ccc4ccccc4c3N3c4ccccc4C(C)(C)c4ccc(-c5cccc6ccccc56)cc43)cc21 ; CC1(C)c2ccccc2N(c2c(N(c3ccc(-c4ccccc4)cc3)c3ccc(-c4cccc5ccccc45)cc3)ccc3ccccc23)c2cc(-c3cccc4ccccc34)ccc21]

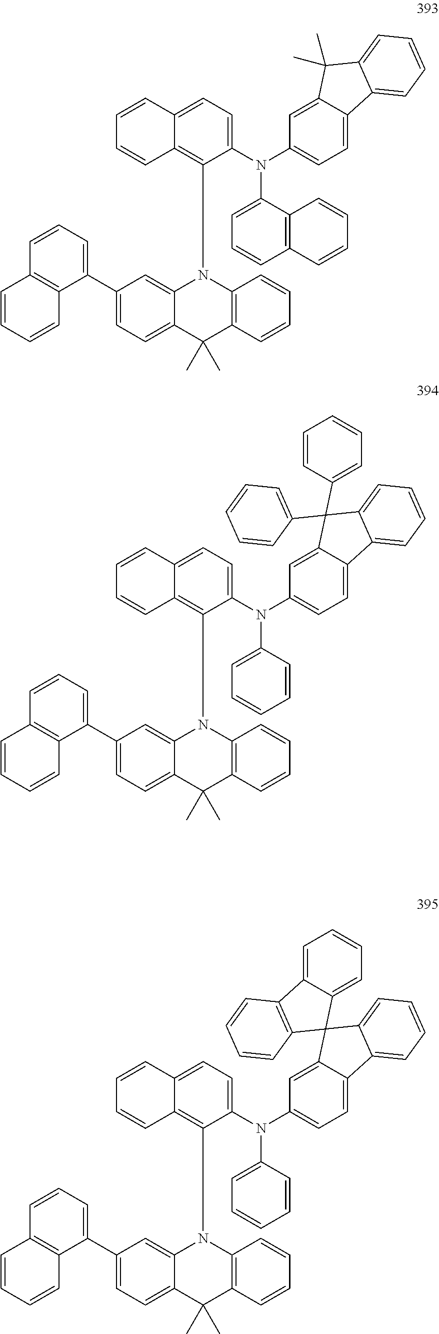 [SMILES: CC1(C)c2ccccc2-c2ccc(N(c3ccc4ccccc4c3N3c4ccccc4C(C)(C)c4ccc(-c5cccc6ccccc56)cc43)c3cccc4ccccc34)cc21 ; CC1(C)c2ccccc2N(c2c(N(c3ccccc3)c3ccc4c(c3)C(c3ccccc3)(c3ccccc3)c3ccccc3-4)ccc3ccccc23)c2cc(-c3cccc4ccccc34)ccc21 ; CC1(C)c2ccccc2N(c2c(N(c3ccccc3)c3ccc4c(c3)C3(c5ccccc5-c5ccccc53)c3ccccc3-4)ccc3ccccc23)c2cc(-c3cccc4ccccc34)ccc21]

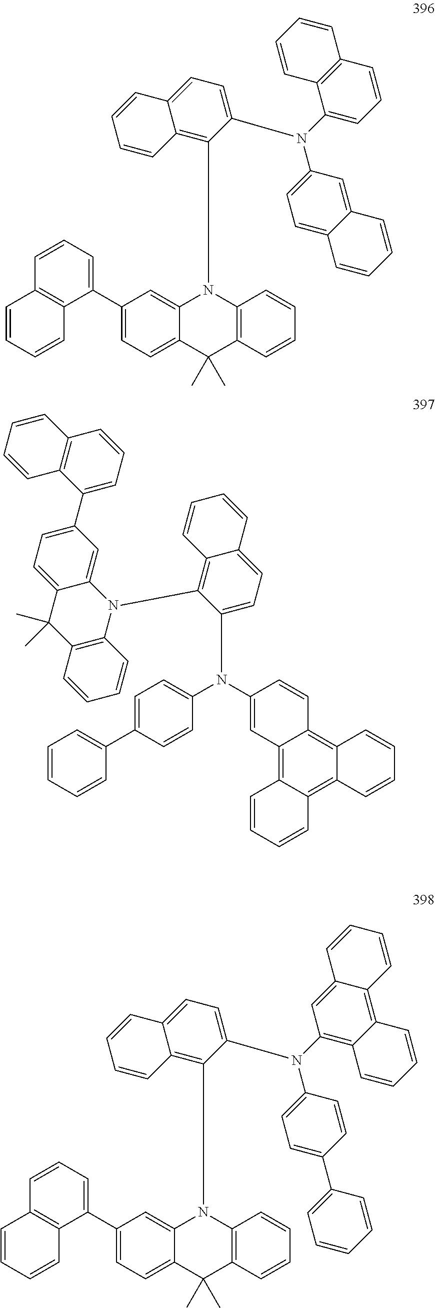 [SMILES: CC1(C)c2ccccc2N(c2c(N(c3ccc4ccccc4c3)c3cccc4ccccc34)ccc3ccccc23)c2cc(-c3cccc4ccccc34)ccc21 ; CC1(C)c2ccccc2N(c2c(N(c3ccc(-c4ccccc4)cc3)c3ccc4c5ccccc5c5ccccc5c4c3)ccc3ccccc23)c2cc(-c3cccc4ccccc34)ccc21 ; CC1(C)c2ccccc2N(c2c(N(c3ccc(-c4ccccc4)cc3)c3cc4ccccc4c4ccccc34)ccc3ccccc23)c2cc(-c3cccc4ccccc34)ccc21]

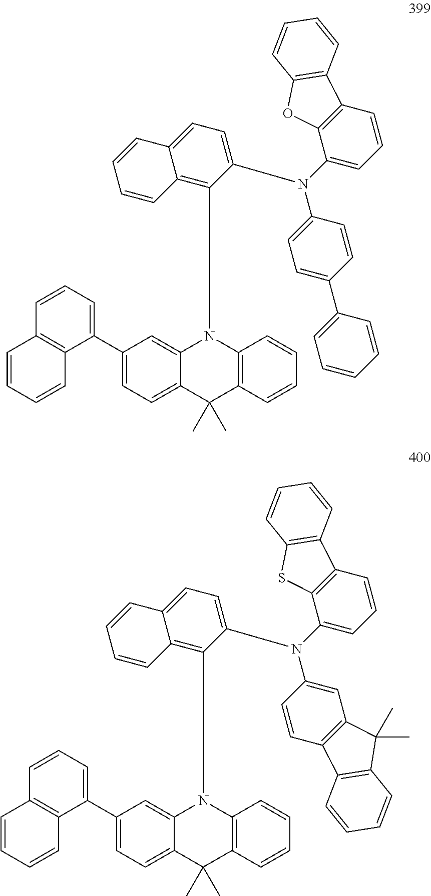 [SMILES: CC1(C)c2ccccc2N(c2c(N(c3ccc(-c4ccccc4)cc3)c3cccc4c3oc3ccccc34)ccc3ccccc23)c2cc(-c3cccc4ccccc34)ccc21 ; CC1(C)c2ccccc2-c2ccc(N(c3ccc4ccccc4c3N3c4ccccc4C(C)(C)c4ccc(-c5cccc6ccccc56)cc43)c3cccc4c3sc3ccccc34)cc21]

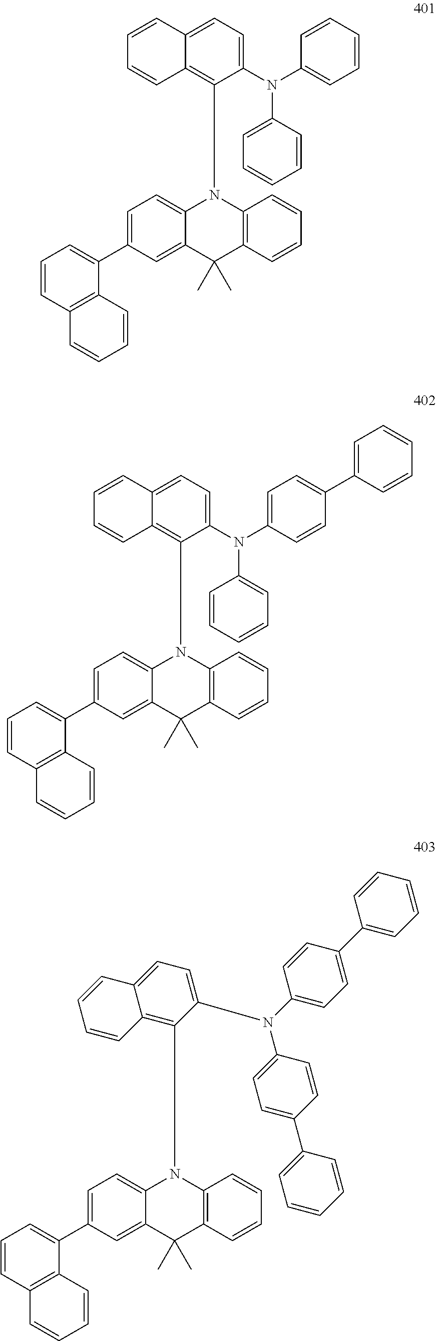 [SMILES: CC1(C)c2ccccc2N(c2c(N(c3ccccc3)c3ccccc3)ccc3ccccc23)c2ccc(-c3cccc4ccccc34)cc21 ; CC1(C)c2ccccc2N(c2c(N(c3ccccc3)c3ccc(-c4ccccc4)cc3)ccc3ccccc23)c2ccc(-c3cccc4ccccc34)cc21 ; CC1(C)c2ccccc2N(c2c(N(c3ccc(-c4ccccc4)cc3)c3ccc(-c4ccccc4)cc3)ccc3ccccc23)c2ccc(-c3cccc4ccccc34)cc21]

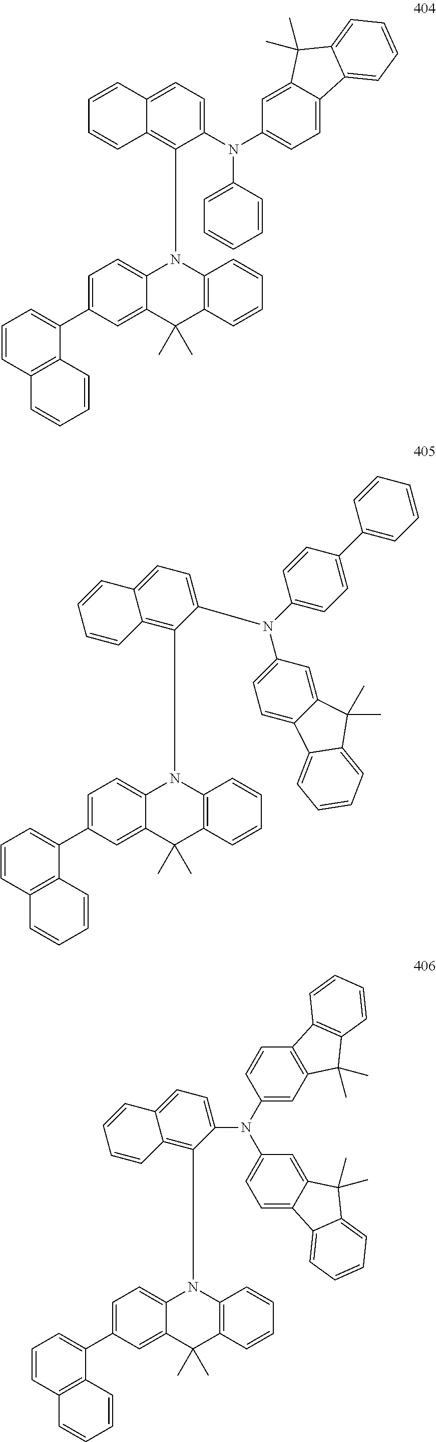 [SMILES: CC1(C)c2ccccc2-c2ccc(N(c3ccccc3)c3ccc4ccccc4c3N3c4ccccc4C(C)(C)c4cc(-c5cccc6ccccc56)ccc43)cc21 ; CC1(C)c2ccccc2-c2ccc(N(c3ccc(-c4ccccc4)cc3)c3ccc4ccccc4c3N3c4ccccc4C(C)(C)c4cc(-c5cccc6ccccc56)ccc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4cc(-c5cccc6ccccc56)ccc43)cc21]

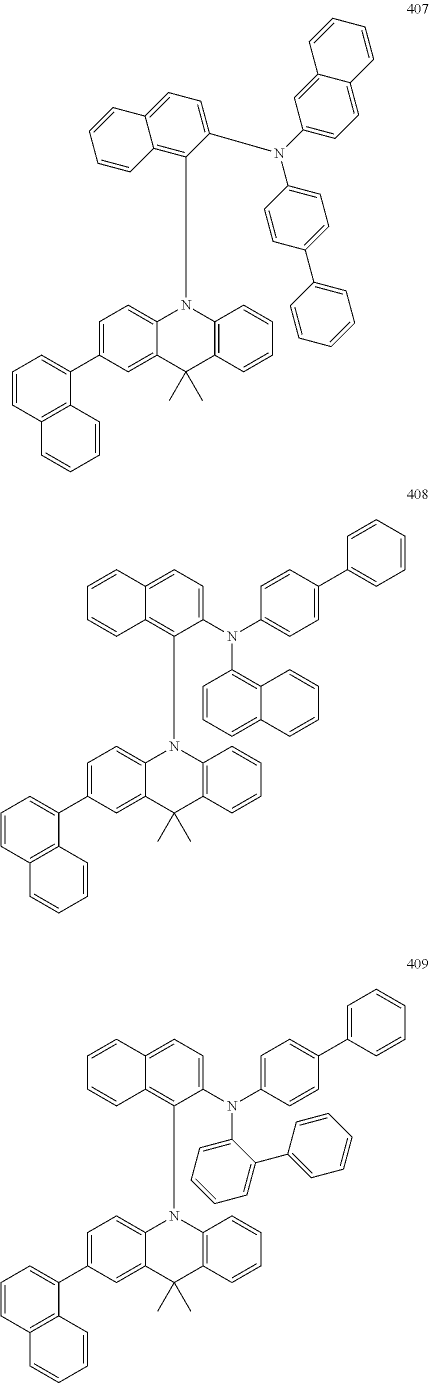 [SMILES: CC1(C)c2ccccc2N(c2c(N(c3ccc(-c4ccccc4)cc3)c3ccc4ccccc4c3)ccc3ccccc23)c2ccc(-c3cccc4ccccc34)cc21 ; CC1(C)c2ccccc2N(c2c(N(c3ccc(-c4ccccc4)cc3)c3cccc4ccccc34)ccc3ccccc23)c2ccc(-c3cccc4ccccc34)cc21 ; CC1(C)c2ccccc2N(c2c(N(c3ccc(-c4ccccc4)cc3)c3ccccc3-c3ccccc3)ccc3ccccc23)c2ccc(-c3cccc4ccccc34)cc21]

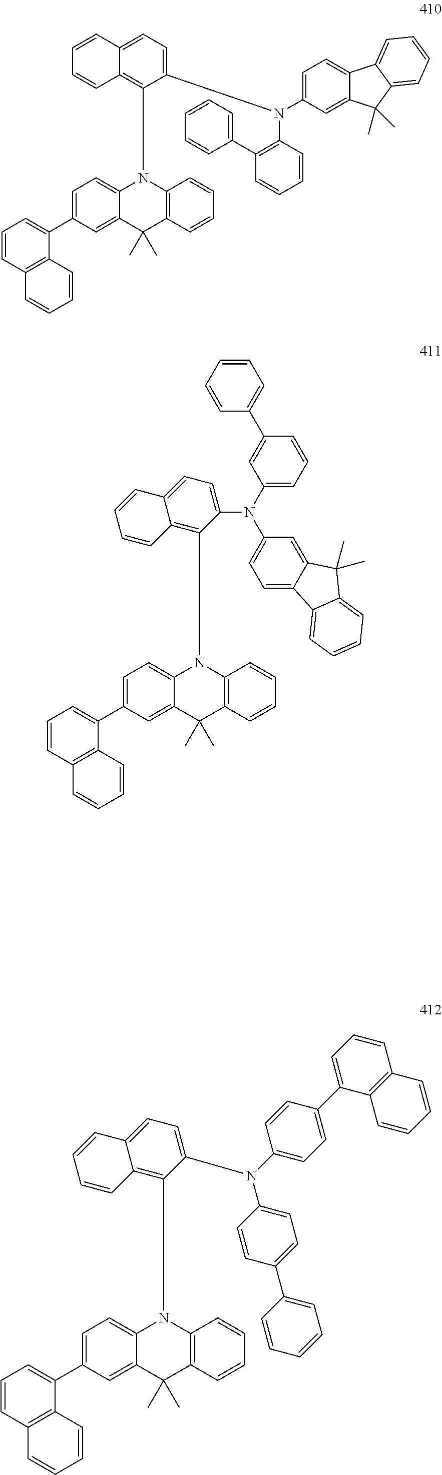 [SMILES: CC1(C)c2ccccc2-c2ccc(N(c3ccccc3-c3ccccc3)c3ccc4ccccc4c3N3c4ccccc4C(C)(C)c4cc(-c5cccc6ccccc56)ccc43)cc21 ; CC1(C)c2ccccc2-c2ccc(N(c3cccc(-c4ccccc4)c3)c3ccc4ccccc4c3N3c4ccccc4C(C)(C)c4cc(-c5cccc6ccccc56)ccc43)cc21 ; CC1(C)c2ccccc2N(c2c(N(c3ccc(-c4ccccc4)cc3)c3ccc(-c4cccc5ccccc45)cc3)ccc3ccccc23)c2ccc(-c3cccc4ccccc34)cc21]

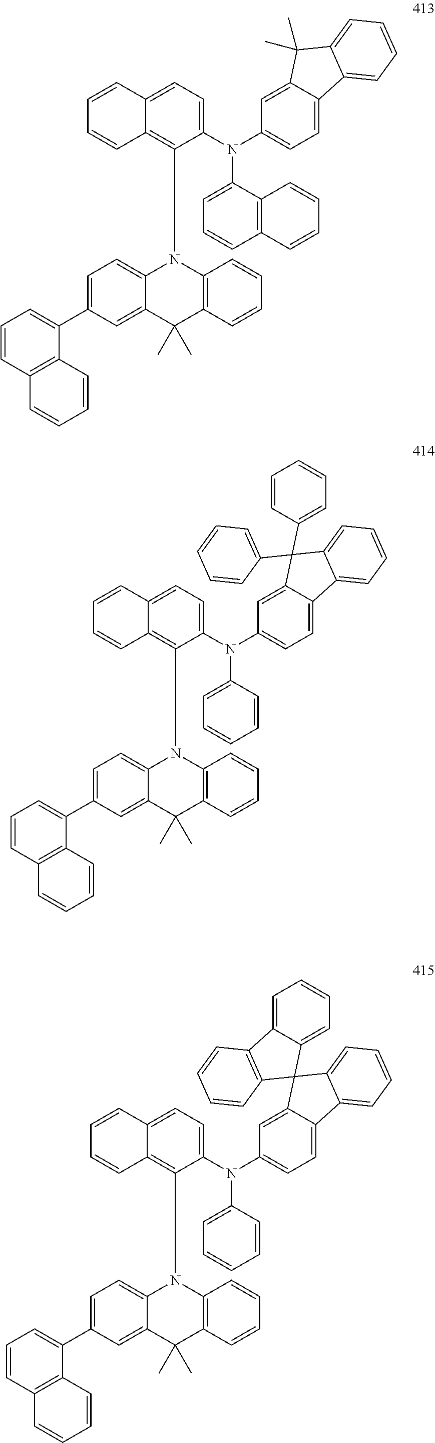 [SMILES: CC1(C)c2ccccc2-c2ccc(N(c3ccc4ccccc4c3N3c4ccccc4C(C)(C)c4cc(-c5cccc6ccccc56)ccc43)c3cccc4ccccc34)cc21 ; CC1(C)c2ccccc2N(c2c(N(c3ccccc3)c3ccc4c(c3)C(c3ccccc3)(c3ccccc3)c3ccccc3-4)ccc3ccccc23)c2ccc(-c3cccc4ccccc34)cc21 ; CC1(C)c2ccccc2N(c2c(N(c3ccccc3)c3ccc4c(c3)C3(c5ccccc5-c5ccccc53)c3ccccc3-4)ccc3ccccc23)c2ccc(-c3cccc4ccccc34)cc21]

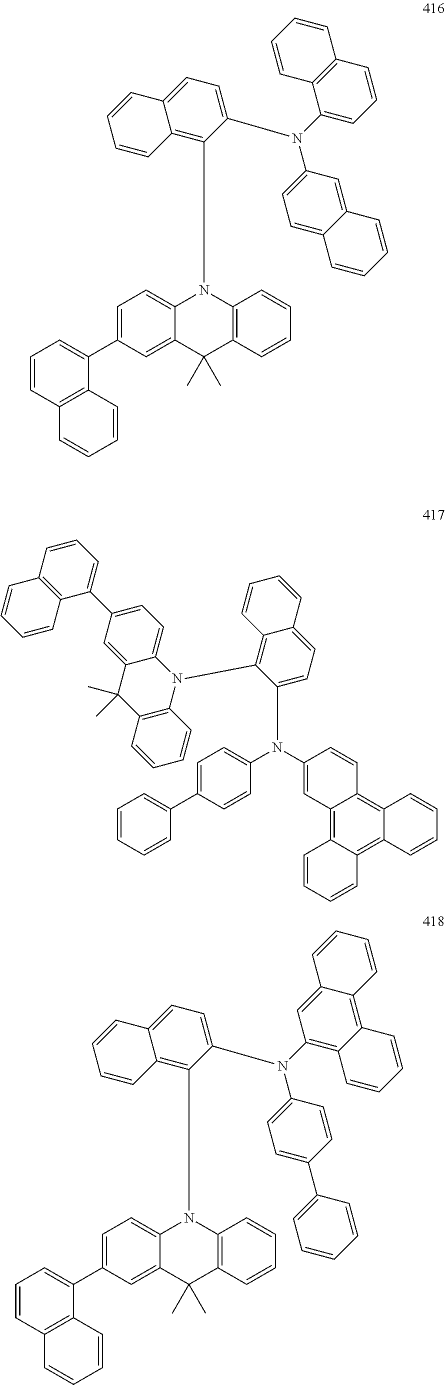

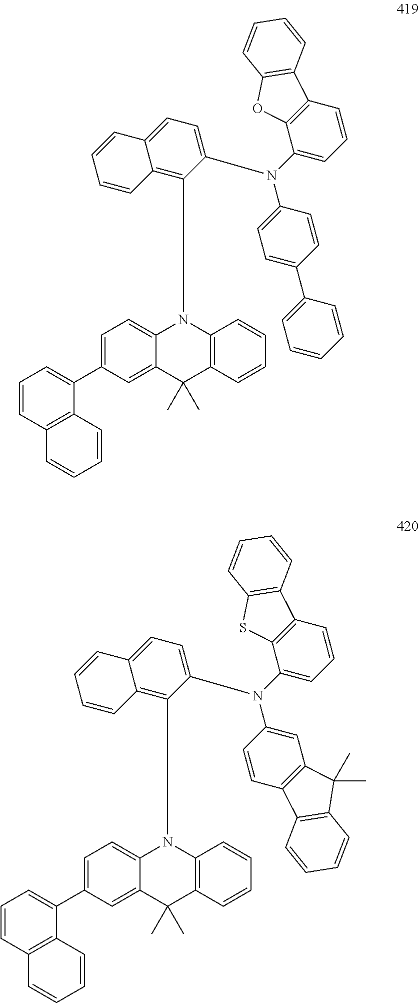 [SMILES: CC1(C)c2ccccc2N(c2c(N(c3ccc(-c4ccccc4)cc3)c3cccc4c3oc3ccccc34)ccc3ccccc23)c2ccc(-c3cccc4ccccc34)cc21 ; CC1(C)c2ccccc2-c2ccc(N(c3ccc4ccccc4c3N3c4ccccc4C(C)(C)c4cc(-c5cccc6ccccc56)ccc43)c3cccc4c3sc3ccccc34)cc21]

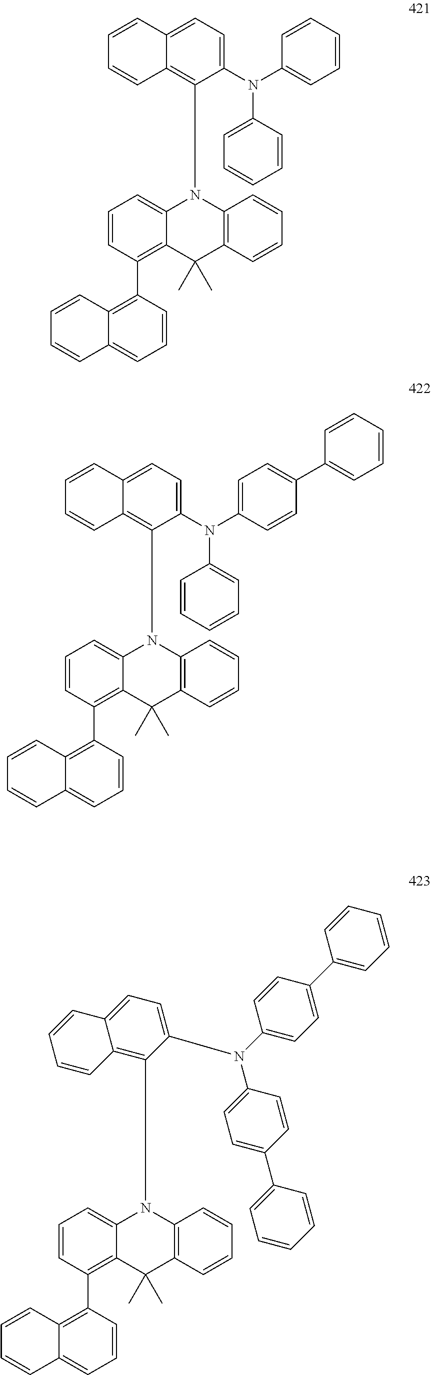 [SMILES: CC1(C)c2ccccc2N(c2c(N(c3ccccc3)c3ccccc3)ccc3ccccc23)c2cccc(-c3cccc4ccccc34)c21 ; CC1(C)c2ccccc2N(c2c(N(c3ccccc3)c3ccc(-c4ccccc4)cc3)ccc3ccccc23)c2cccc(-c3cccc4ccccc34)c21 ; CC1(C)c2ccccc2N(c2c(N(c3ccc(-c4ccccc4)cc3)c3ccc(-c4ccccc4)cc3)ccc3ccccc23)c2cccc(-c3cccc4ccccc34)c21]

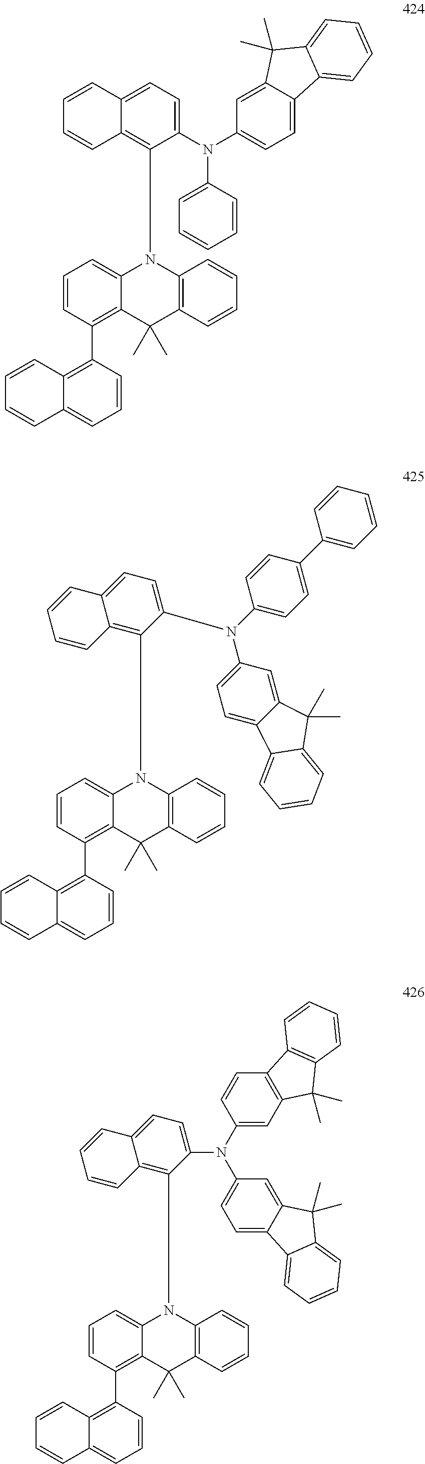 [SMILES: CC1(C)c2ccccc2-c2ccc(N(c3ccccc3)c3ccc4ccccc4c3N3c4ccccc4C(C)(C)c4c(-c5cccc6ccccc56)cccc43)cc21 ; CC1(C)c2ccccc2-c2ccc(N(c3ccc(-c4ccccc4)cc3)c3ccc4ccccc4c3N3c4ccccc4C(C)(C)c4c(-c5cccc6ccccc56)cccc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4c(-c5cccc6ccccc56)cccc43)cc21]

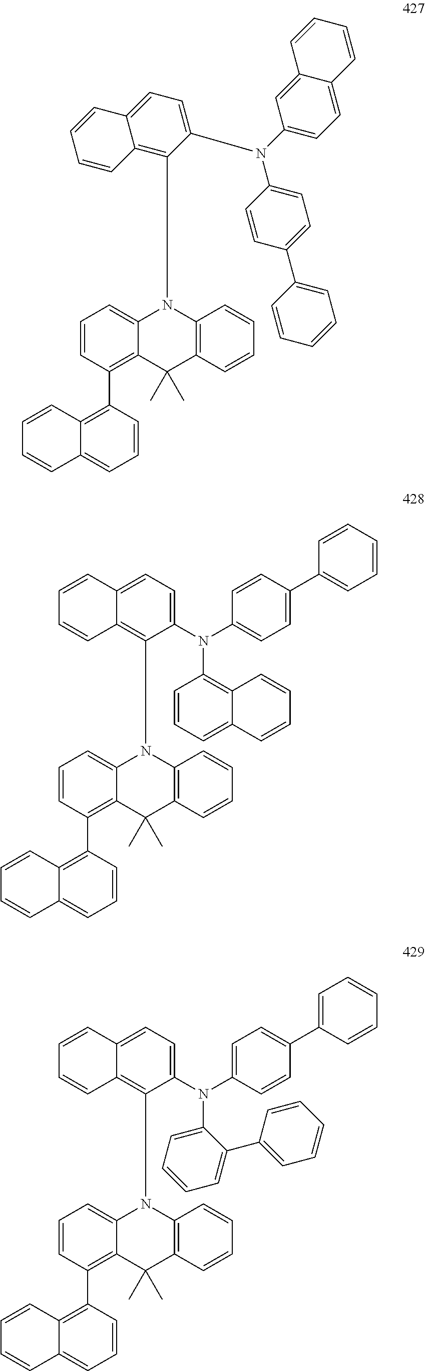 [SMILES: CC1(C)c2ccccc2N(c2c(N(c3ccc(-c4ccccc4)cc3)c3ccc4ccccc4c3)ccc3ccccc23)c2cccc(-c3cccc4ccccc34)c21 ; CC1(C)c2ccccc2N(c2c(N(c3ccc(-c4ccccc4)cc3)c3cccc4ccccc34)ccc3ccccc23)c2cccc(-c3cccc4ccccc34)c21 ; CC1(C)c2ccccc2N(c2c(N(c3ccc(-c4ccccc4)cc3)c3ccccc3-c3ccccc3)ccc3ccccc23)c2cccc(-c3cccc4ccccc34)c21]

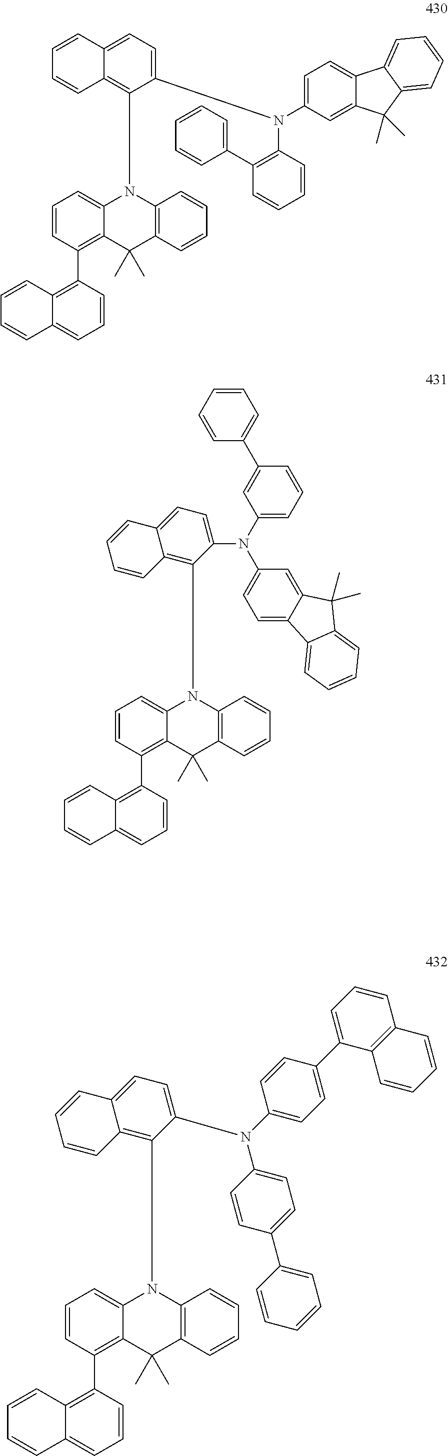 [SMILES: CC1(C)c2ccccc2-c2ccc(N(c3ccccc3-c3ccccc3)c3ccc4ccccc4c3N3c4ccccc4C(C)(C)c4c(-c5cccc6ccccc56)cccc43)cc21 ; CC1(C)c2ccccc2-c2ccc(N(c3cccc(-c4ccccc4)c3)c3ccc4ccccc4c3N3c4ccccc4C(C)(C)c4c(-c5cccc6ccccc56)cccc43)cc21 ; CC1(C)c2ccccc2N(c2c(N(c3ccc(-c4ccccc4)cc3)c3ccc(-c4cccc5ccccc45)cc3)ccc3ccccc23)c2cccc(-c3cccc4ccccc34)c21]

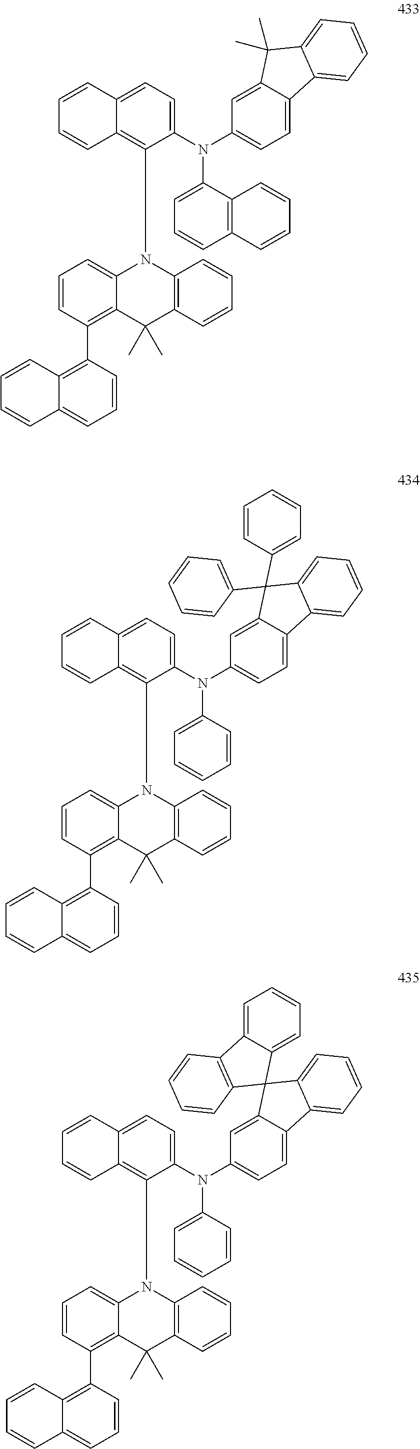 [SMILES: CC1(C)c2ccccc2-c2ccc(N(c3ccc4ccccc4c3N3c4ccccc4C(C)(C)c4c(-c5cccc6ccccc56)cccc43)c3cccc4ccccc34)cc21 ; CC1(C)c2ccccc2N(c2c(N(c3ccccc3)c3ccc4c(c3)C(c3ccccc3)(c3ccccc3)c3ccccc3-4)ccc3ccccc23)c2cccc(-c3cccc4ccccc34)c21 ; CC1(C)c2ccccc2N(c2c(N(c3ccccc3)c3ccc4c(c3)C3(c5ccccc5-c5ccccc53)c3ccccc3-4)ccc3ccccc23)c2cccc(-c3cccc4ccccc34)c21]

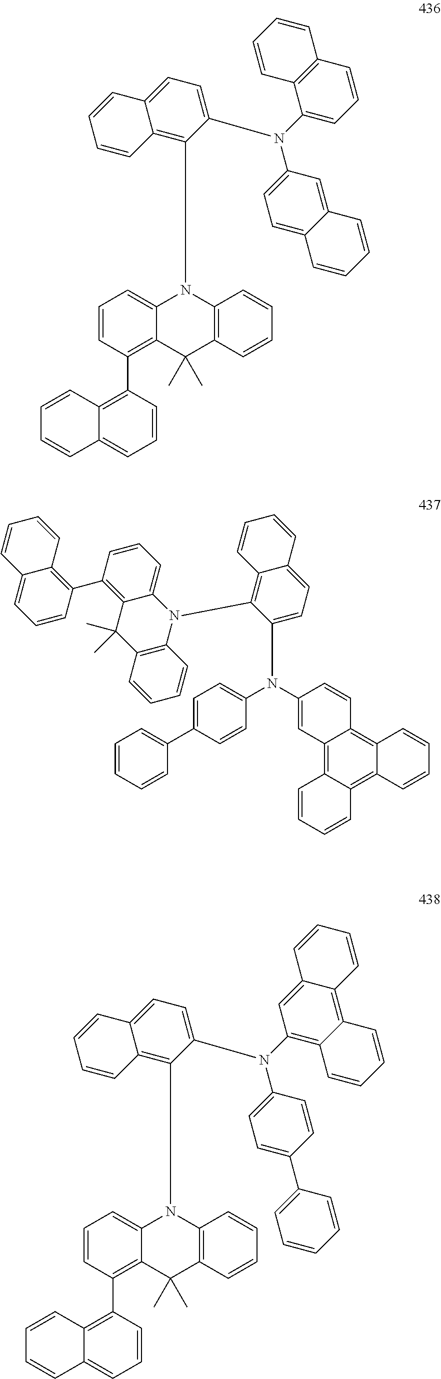 [SMILES: CC1(C)c2ccccc2N(c2c(N(c3ccc4ccccc4c3)c3cccc4ccccc34)ccc3ccccc23)c2cccc(-c3cccc4ccccc34)c21 ; CC1(C)c2ccccc2N(c2c(N(c3ccc(-c4ccccc4)cc3)c3ccc4c5ccccc5c5ccccc5c4c3)ccc3ccccc23)c2cccc(-c3cccc4ccccc34)c21 ; CC1(C)c2ccccc2N(c2c(N(c3ccc(-c4ccccc4)cc3)c3cc4ccccc4c4ccccc34)ccc3ccccc23)c2cccc(-c3cccc4ccccc34)c21]

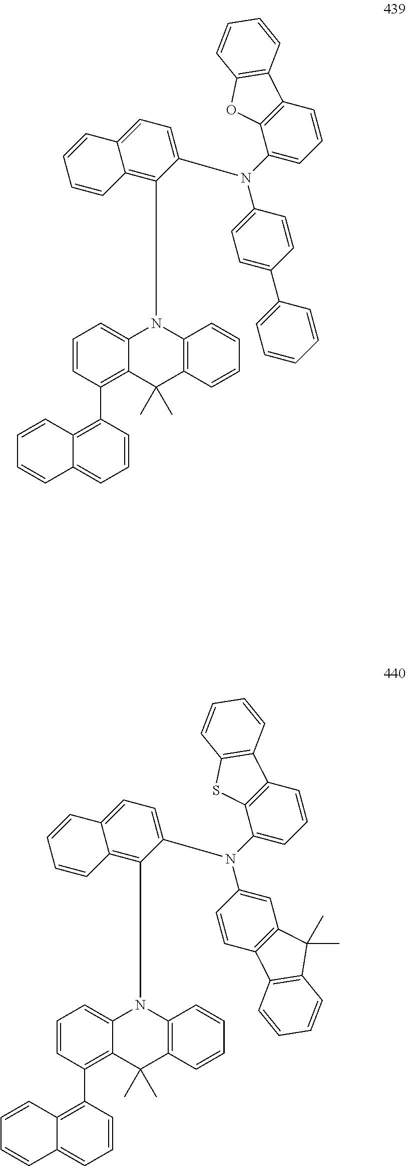 [SMILES: CC1(C)c2ccccc2N(c2c(N(c3ccc(-c4ccccc4)cc3)c3cccc4c3oc3ccccc34)ccc3ccccc23)c2cccc(-c3cccc4ccccc34)c21 ; CC1(C)c2ccccc2-c2ccc(N(c3ccc4ccccc4c3N3c4ccccc4C(C)(C)c4c(-c5cccc6ccccc56)cccc43)c3cccc4c3sc3ccccc34)cc21]

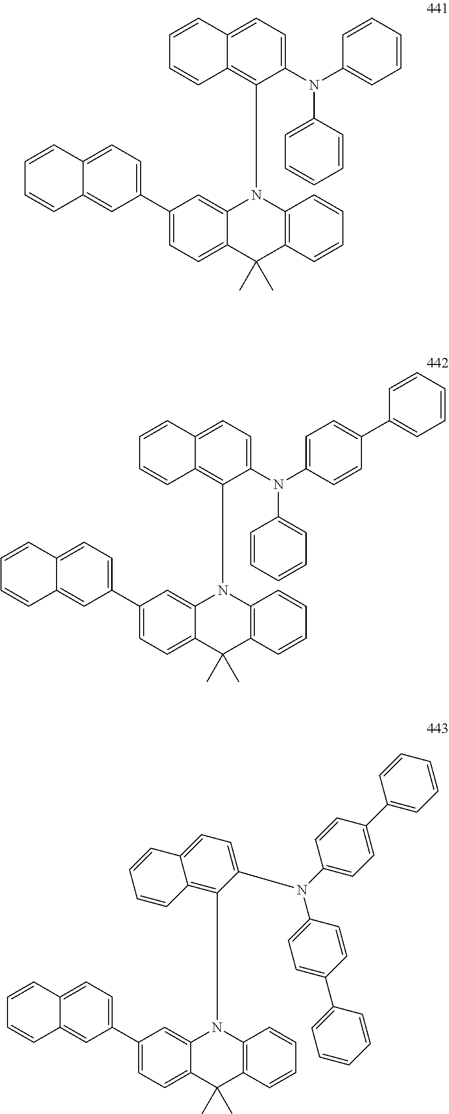 [SMILES: CC1(C)c2ccccc2N(c2c(N(c3ccccc3)c3ccccc3)ccc3ccccc23)c2cc(-c3ccc4ccccc4c3)ccc21 ; CC1(C)c2ccccc2N(c2c(N(c3ccccc3)c3ccc(-c4ccccc4)cc3)ccc3ccccc23)c2cc(-c3ccc4ccccc4c3)ccc21 ; CC1(C)c2ccccc2N(c2c(N(c3ccc(-c4ccccc4)cc3)c3ccc(-c4ccccc4)cc3)ccc3ccccc23)c2cc(-c3ccc4ccccc4c3)ccc21]

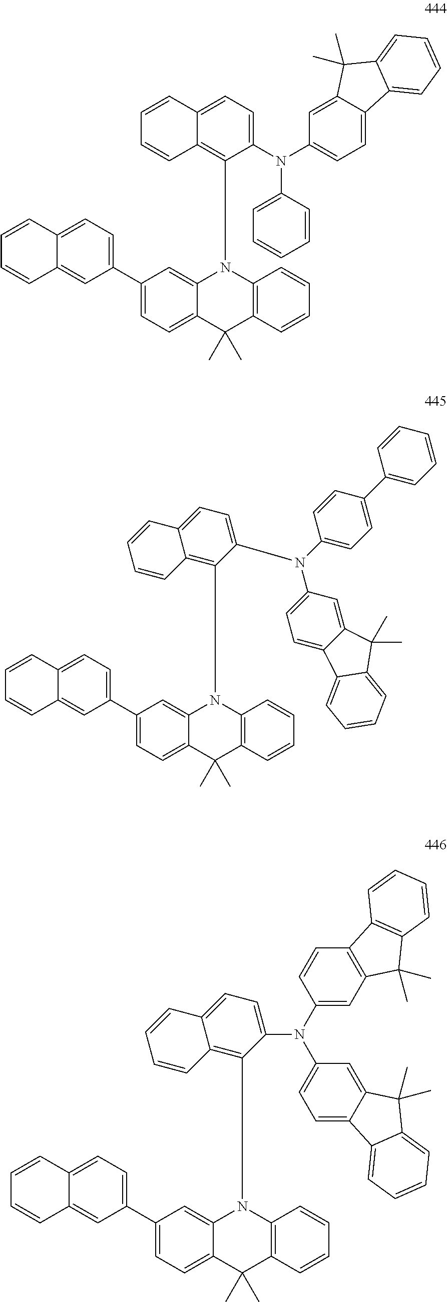 [SMILES: CC1(C)c2ccccc2-c2ccc(N(c3ccccc3)c3ccc4ccccc4c3N3c4ccccc4C(C)(C)c4ccc(-c5ccc6ccccc6c5)cc43)cc21 ; CC1(C)c2ccccc2-c2ccc(N(c3ccc(-c4ccccc4)cc3)c3ccc4ccccc4c3N3c4ccccc4C(C)(C)c4ccc(-c5ccc6ccccc6c5)cc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4ccc(-c5ccc6ccccc6c5)cc43)cc21]

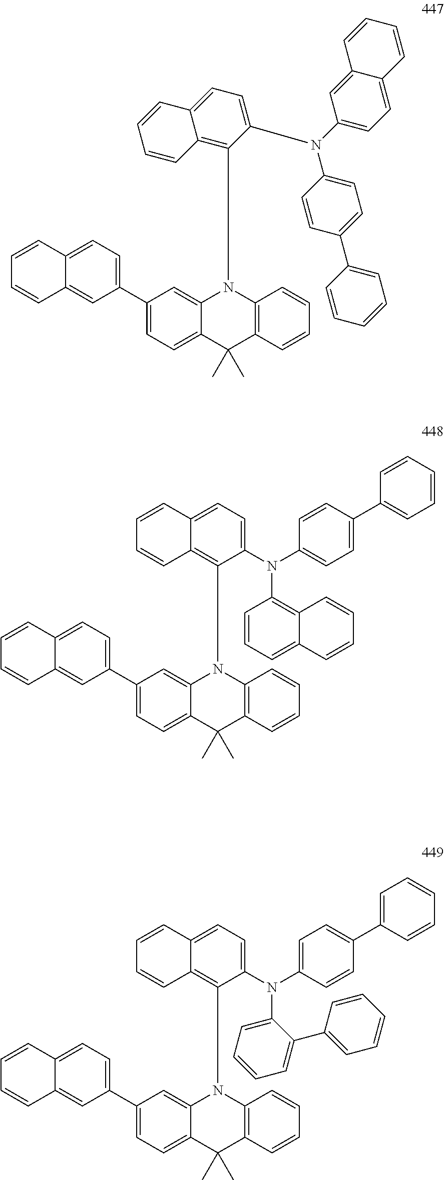 [SMILES: CC1(C)c2ccccc2N(c2c(N(c3ccc(-c4ccccc4)cc3)c3ccc4ccccc4c3)ccc3ccccc23)c2cc(-c3ccc4ccccc4c3)ccc21 ; CC1(C)c2ccccc2N(c2c(N(c3ccc(-c4ccccc4)cc3)c3cccc4ccccc34)ccc3ccccc23)c2cc(-c3ccc4ccccc4c3)ccc21 ; CC1(C)c2ccccc2N(c2c(N(c3ccc(-c4ccccc4)cc3)c3ccccc3-c3ccccc3)ccc3ccccc23)c2cc(-c3ccc4ccccc4c3)ccc21]

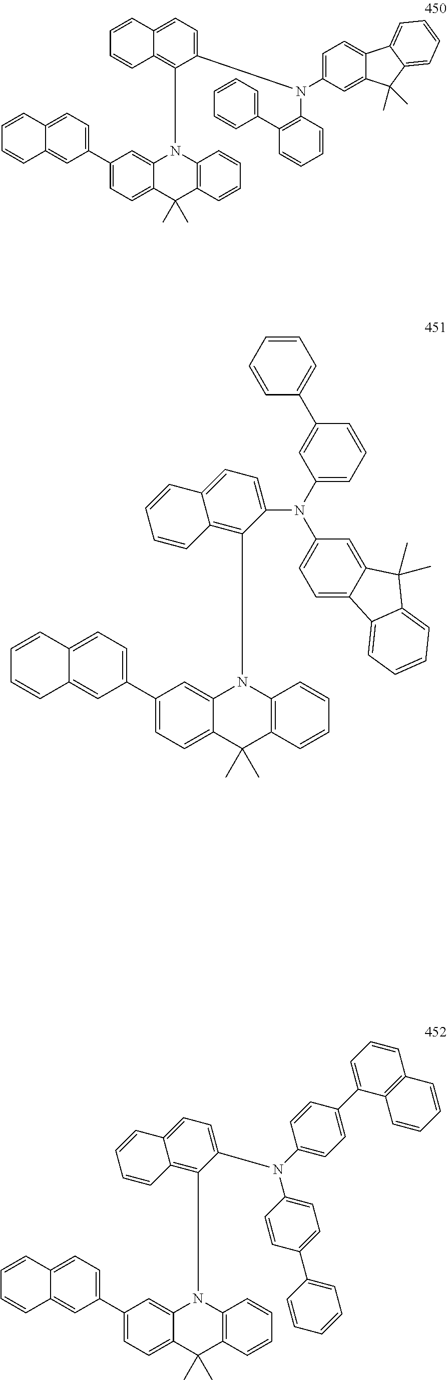 [SMILES: CC1(C)c2ccccc2-c2ccc(N(c3ccccc3-c3ccccc3)c3ccc4ccccc4c3N3c4ccccc4C(C)(C)c4ccc(-c5ccc6ccccc6c5)cc43)cc21 ; CC1(C)c2ccccc2-c2ccc(N(c3cccc(-c4ccccc4)c3)c3ccc4ccccc4c3N3c4ccccc4C(C)(C)c4ccc(-c5ccc6ccccc6c5)cc43)cc21 ; CC1(C)c2ccccc2N(c2c(N(c3ccc(-c4ccccc4)cc3)c3ccc(-c4cccc5ccccc45)cc3)ccc3ccccc23)c2cc(-c3ccc4ccccc4c3)ccc21]

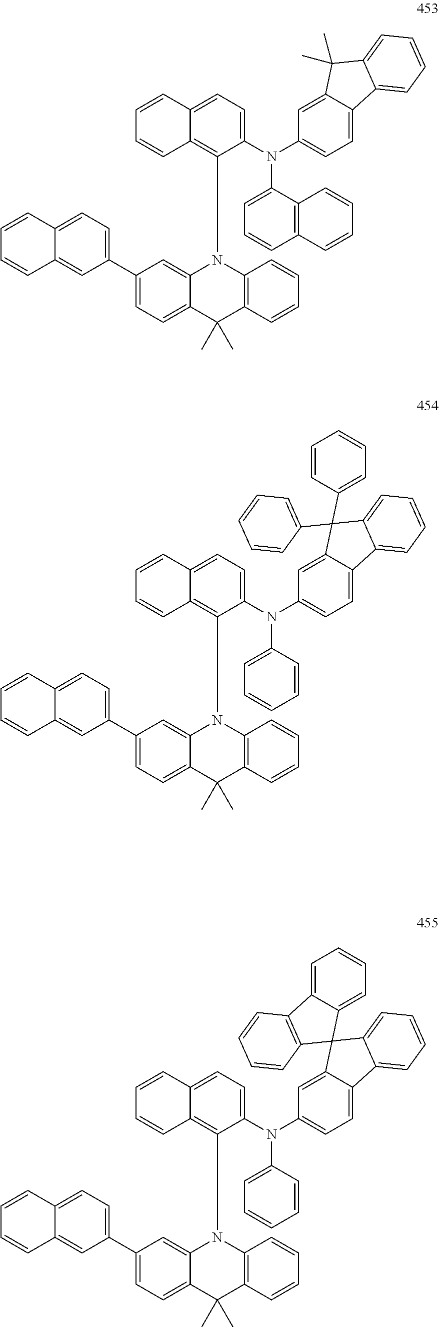 [SMILES: CC1(C)c2ccccc2-c2ccc(N(c3ccc4ccccc4c3N3c4ccccc4C(C)(C)c4ccc(-c5ccc6ccccc6c5)cc43)c3cccc4ccccc34)cc21 ; CC1(C)c2ccccc2N(c2c(N(c3ccccc3)c3ccc4c(c3)C(c3ccccc3)(c3ccccc3)c3ccccc3-4)ccc3ccccc23)c2cc(-c3ccc4ccccc4c3)ccc21 ; CC1(C)c2ccccc2N(c2c(N(c3ccccc3)c3ccc4c(c3)C3(c5ccccc5-c5ccccc53)c3ccccc3-4)ccc3ccccc23)c2cc(-c3ccc4ccccc4c3)ccc21]

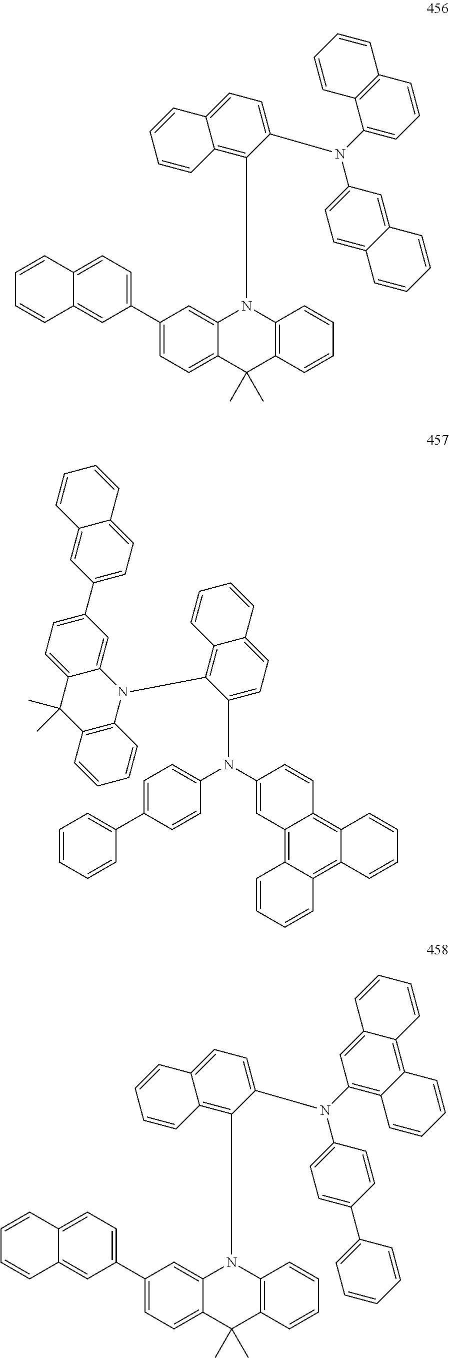 [SMILES: CC1(C)c2ccccc2N(c2c(N(c3ccc4ccccc4c3)c3cccc4ccccc34)ccc3ccccc23)c2cc(-c3ccc4ccccc4c3)ccc21 ; CC1(C)c2ccccc2N(c2c(N(c3ccc(-c4ccccc4)cc3)c3ccc4c5ccccc5c5ccccc5c4c3)ccc3ccccc23)c2cc(-c3ccc4ccccc4c3)ccc21 ; CC1(C)c2ccccc2N(c2c(N(c3ccc(-c4ccccc4)cc3)c3cc4ccccc4c4ccccc34)ccc3ccccc23)c2cc(-c3ccc4ccccc4c3)ccc21]

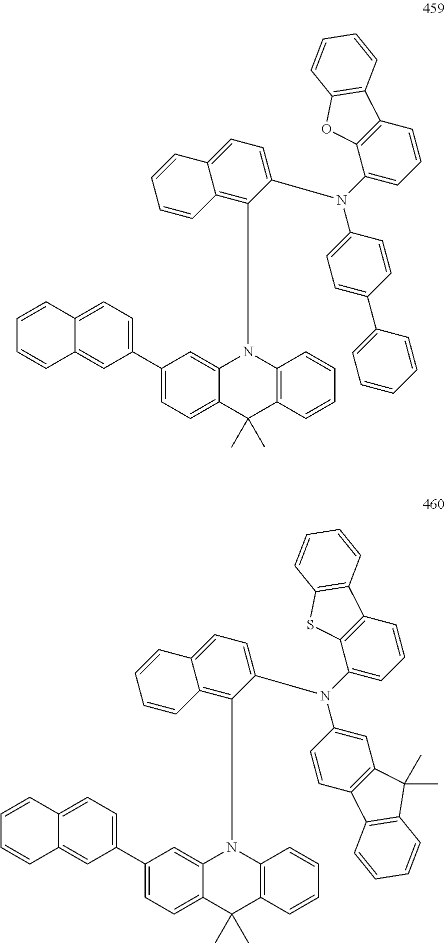 [SMILES: CC1(C)c2ccccc2N(c2c(N(c3ccc(-c4ccccc4)cc3)c3cccc4c3oc3ccccc34)ccc3ccccc23)c2cc(-c3ccc4ccccc4c3)ccc21 ; CC1(C)c2ccccc2-c2ccc(N(c3ccc4ccccc4c3N3c4ccccc4C(C)(C)c4ccc(-c5ccc6ccccc6c5)cc43)c3cccc4c3sc3ccccc34)cc21]

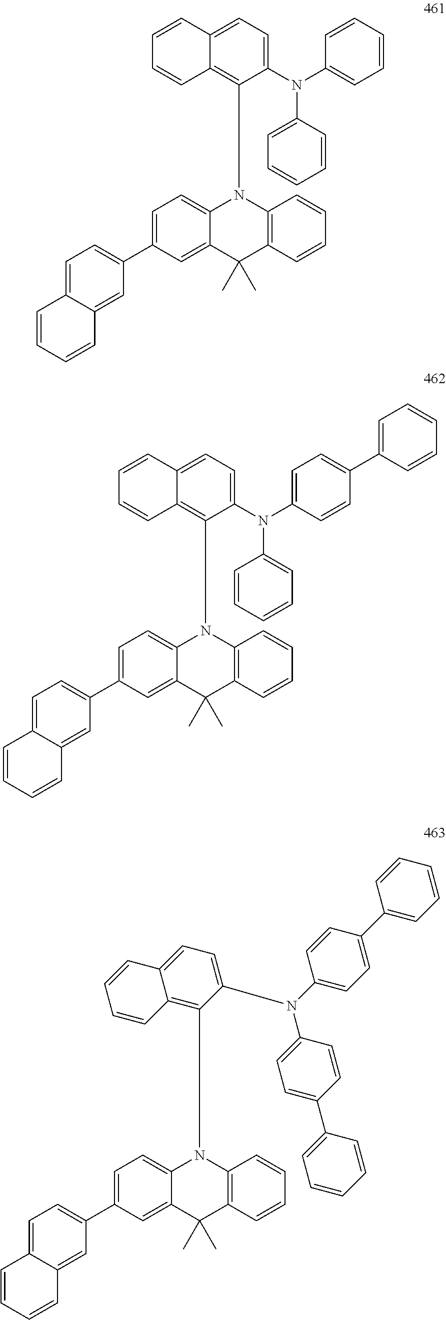 [SMILES: CC1(C)c2ccccc2N(c2c(N(c3ccccc3)c3ccccc3)ccc3ccccc23)c2ccc(-c3ccc4ccccc4c3)cc21 ; CC1(C)c2ccccc2N(c2c(N(c3ccccc3)c3ccc(-c4ccccc4)cc3)ccc3ccccc23)c2ccc(-c3ccc4ccccc4c3)cc21 ; CC1(C)c2ccccc2N(c2c(N(c3ccc(-c4ccccc4)cc3)c3ccc(-c4ccccc4)cc3)ccc3ccccc23)c2ccc(-c3ccc4ccccc4c3)cc21]

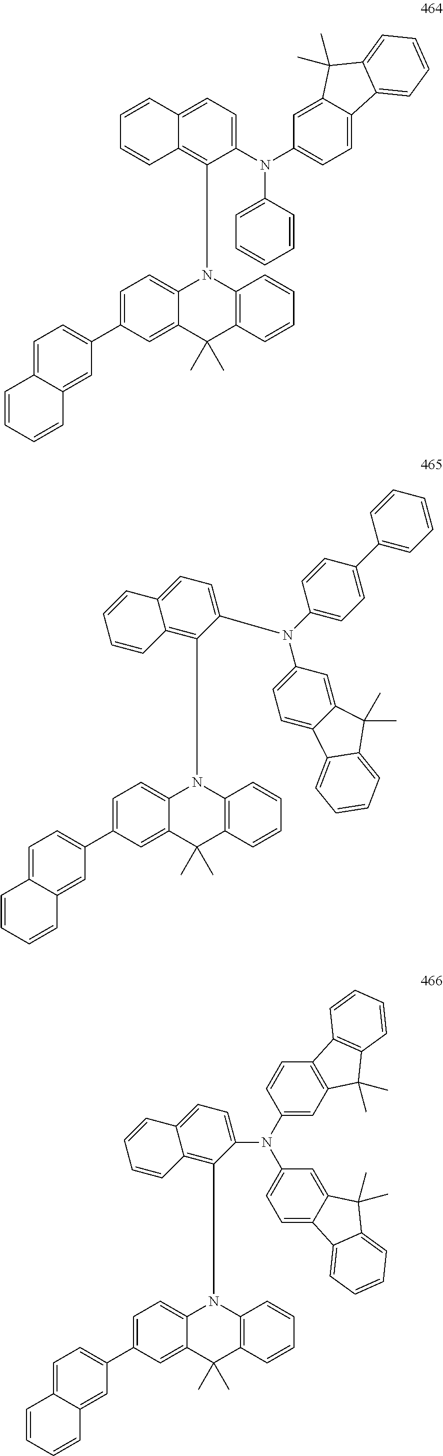 [SMILES: CC1(C)c2ccccc2-c2ccc(N(c3ccccc3)c3ccc4ccccc4c3N3c4ccccc4C(C)(C)c4cc(-c5ccc6ccccc6c5)ccc43)cc21 ; CC1(C)c2ccccc2-c2ccc(N(c3ccc(-c4ccccc4)cc3)c3ccc4ccccc4c3N3c4ccccc4C(C)(C)c4cc(-c5ccc6ccccc6c5)ccc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4cc(-c5ccc6ccccc6c5)ccc43)cc21]

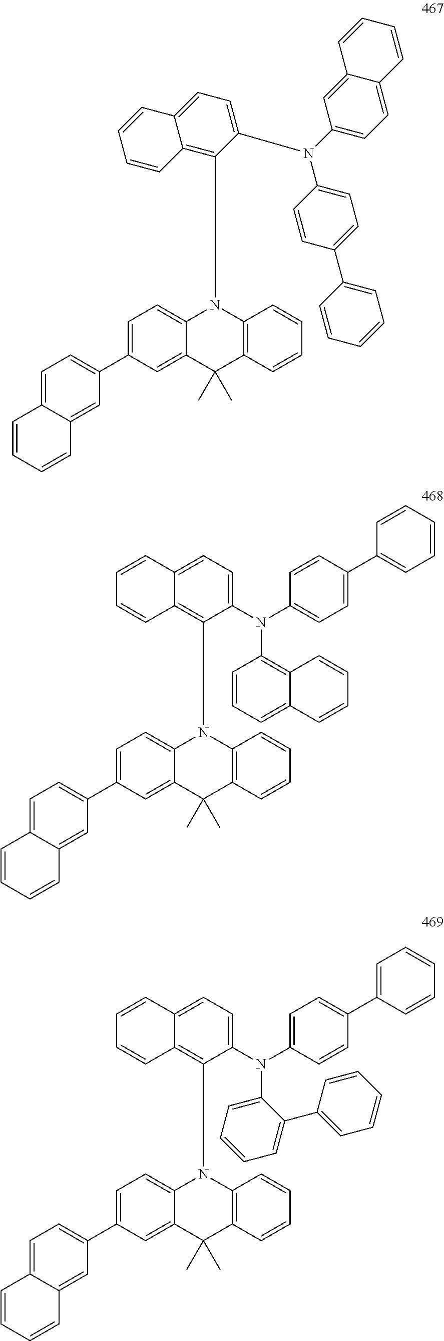 [SMILES: CC1(C)c2ccccc2N(c2c(N(c3ccc(-c4ccccc4)cc3)c3ccc4ccccc4c3)ccc3ccccc23)c2ccc(-c3ccc4ccccc4c3)cc21 ; CC1(C)c2ccccc2N(c2c(N(c3ccc(-c4ccccc4)cc3)c3cccc4ccccc34)ccc3ccccc23)c2ccc(-c3ccc4ccccc4c3)cc21 ; CC1(C)c2ccccc2N(c2c(N(c3ccc(-c4ccccc4)cc3)c3ccccc3-c3ccccc3)ccc3ccccc23)c2ccc(-c3ccc4ccccc4c3)cc21]

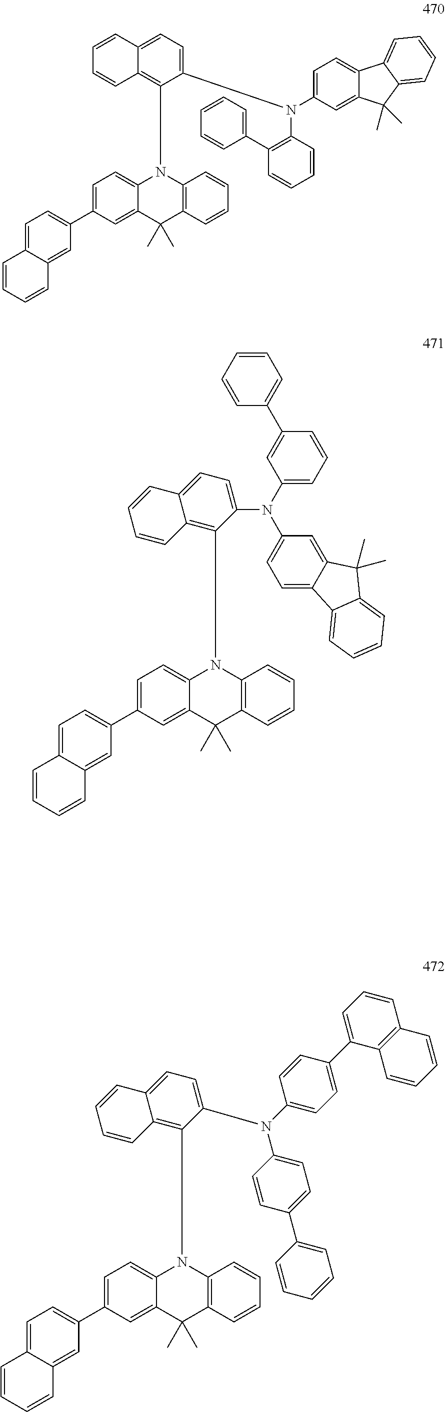 [SMILES: CC1(C)c2ccccc2-c2ccc(N(c3ccccc3-c3ccccc3)c3ccc4ccccc4c3N3c4ccccc4C(C)(C)c4cc(-c5ccc6ccccc6c5)ccc43)cc21 ; CC1(C)c2ccccc2-c2ccc(N(c3cccc(-c4ccccc4)c3)c3ccc4ccccc4c3N3c4ccccc4C(C)(C)c4cc(-c5ccc6ccccc6c5)ccc43)cc21 ; CC1(C)c2ccccc2N(c2c(N(c3ccc(-c4ccccc4)cc3)c3ccc(-c4cccc5ccccc45)cc3)ccc3ccccc23)c2ccc(-c3ccc4ccccc4c3)cc21]

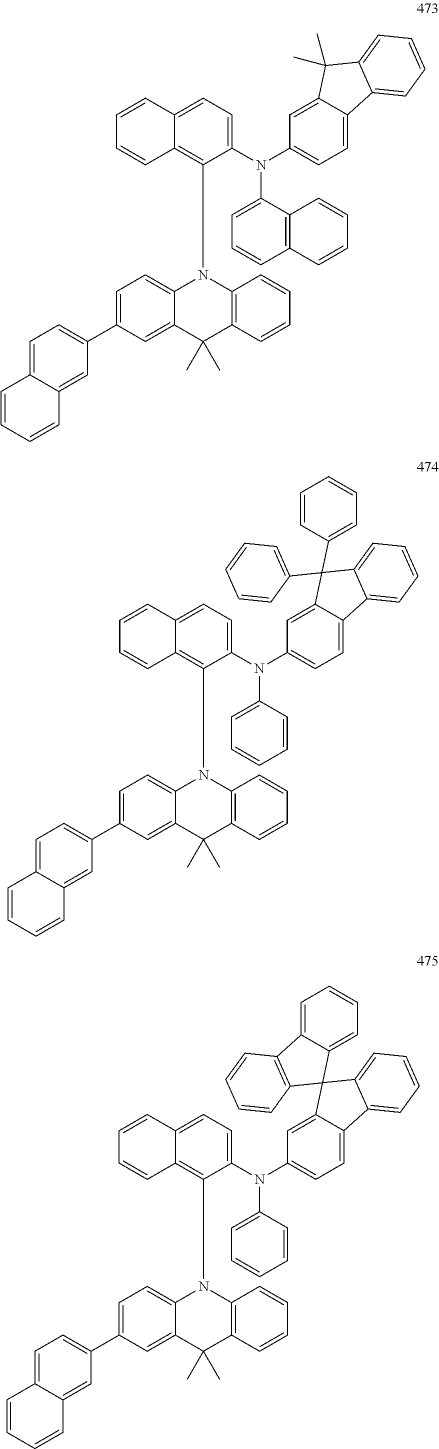 [SMILES: CC1(C)c2ccccc2-c2ccc(N(c3ccc4ccccc4c3N3c4ccccc4C(C)(C)c4cc(-c5ccc6ccccc6c5)ccc43)c3cccc4ccccc34)cc21 ; CC1(C)c2ccccc2N(c2c(N(c3ccccc3)c3ccc4c(c3)C(c3ccccc3)(c3ccccc3)c3ccccc3-4)ccc3ccccc23)c2ccc(-c3ccc4ccccc4c3)cc21 ; CC1(C)c2ccccc2N(c2c(N(c3ccccc3)c3ccc4c(c3)C3(c5ccccc5-c5ccccc53)c3ccccc3-4)ccc3ccccc23)c2ccc(-c3ccc4ccccc4c3)cc21]

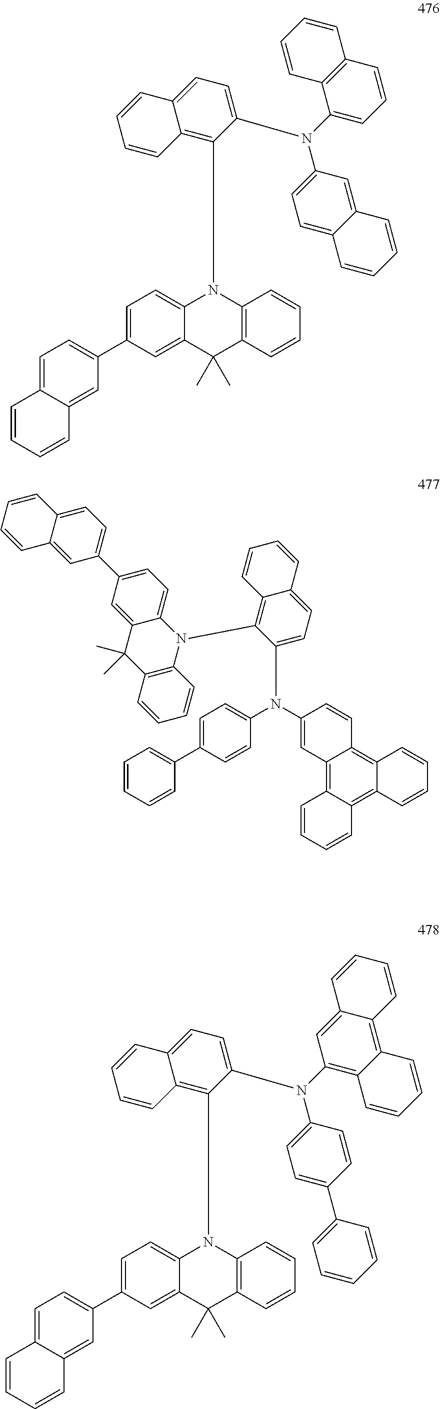

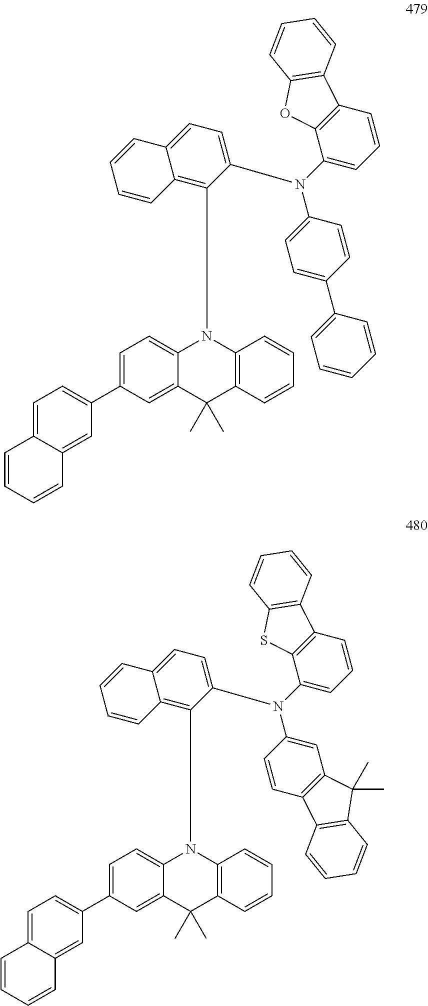 [SMILES: CC1(C)c2ccccc2N(c2c(N(c3ccc(-c4ccccc4)cc3)c3cccc4c3oc3ccccc34)ccc3ccccc23)c2ccc(-c3ccc4ccccc4c3)cc21 ; CC1(C)c2ccccc2-c2ccc(N(c3ccc4ccccc4c3N3c4ccccc4C(C)(C)c4cc(-c5ccc6ccccc6c5)ccc43)c3cccc4c3sc3ccccc34)cc21]

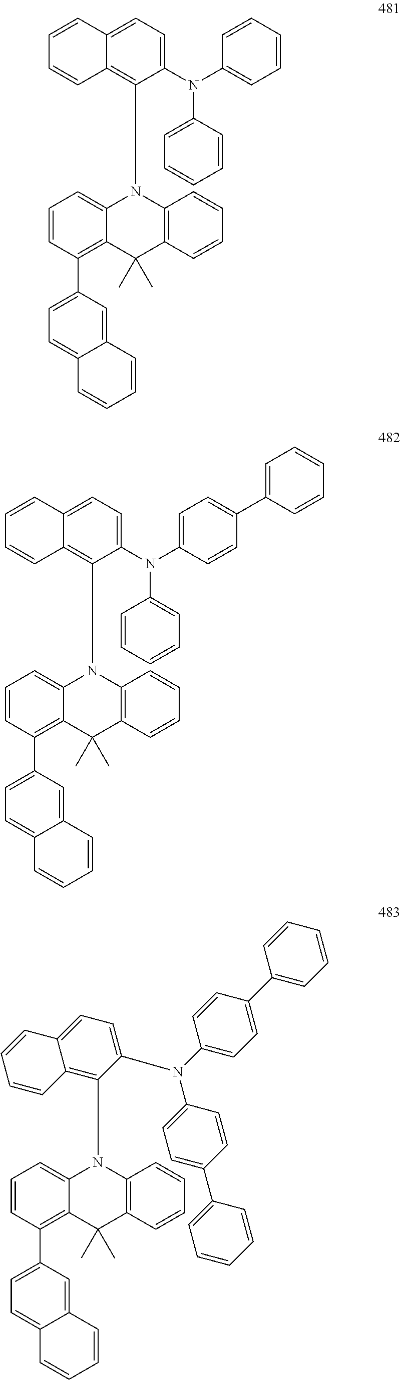 [SMILES: CC1(C)c2ccccc2N(c2c(N(c3ccccc3)c3ccccc3)ccc3ccccc23)c2cccc(-c3ccc4ccccc4c3)c21 ; CC1(C)c2ccccc2N(c2c(N(c3ccccc3)c3ccc(-c4ccccc4)cc3)ccc3ccccc23)c2cccc(-c3ccc4ccccc4c3)c21 ; CC1(C)c2ccccc2N(c2c(N(c3ccc(-c4ccccc4)cc3)c3ccc(-c4ccccc4)cc3)ccc3ccccc23)c2cccc(-c3ccc4ccccc4c3)c21]

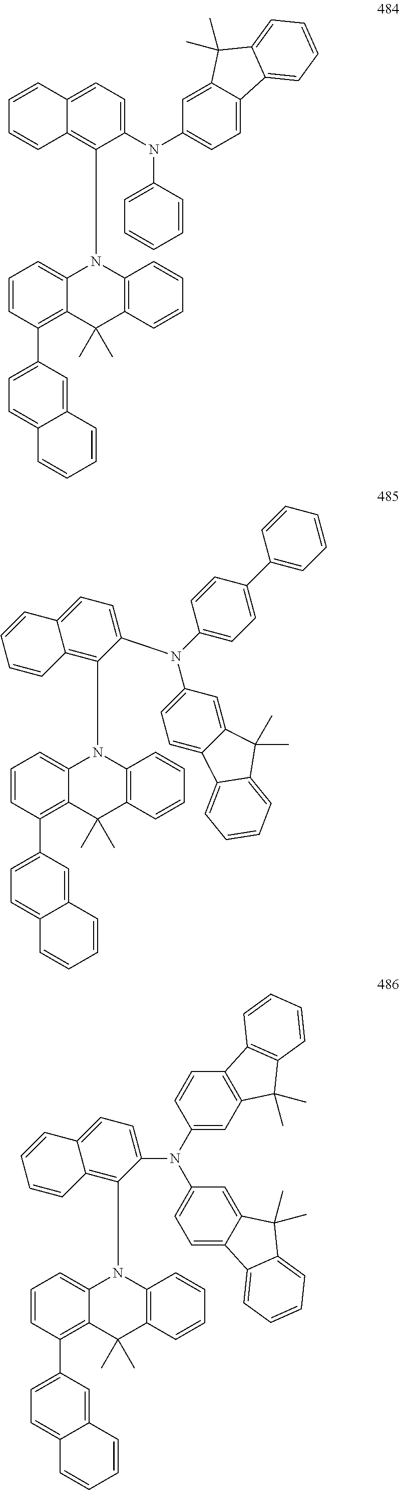 [SMILES: CC1(C)c2ccccc2-c2ccc(N(c3ccccc3)c3ccc4ccccc4c3N3c4ccccc4C(C)(C)c4c(-c5ccc6ccccc6c5)cccc43)cc21 ; CC1(C)c2ccccc2-c2ccc(N(c3ccc(-c4ccccc4)cc3)c3ccc4ccccc4c3N3c4ccccc4C(C)(C)c4c(-c5ccc6ccccc6c5)cccc43)cc21 ; CC1(C)c2ccccc2-c2ccc(N(c3ccc4c(c3)C(C)(C)c3ccccc3-4)c3ccc4ccccc4c3N3c4ccccc4C(C)(C)c4c(-c5ccc6ccccc6c5)cccc43)cc21]

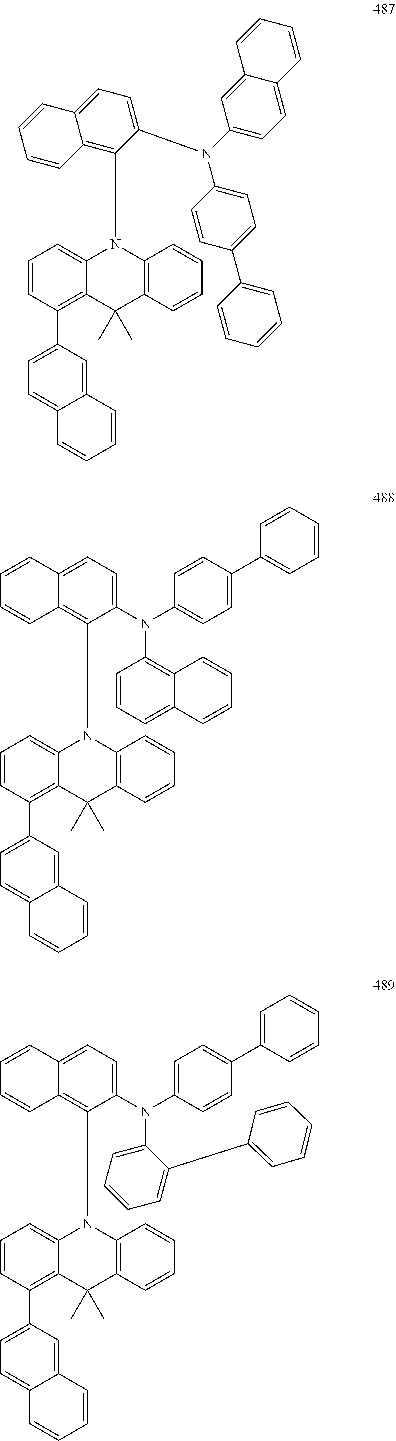 [SMILES: CC1(C)c2ccccc2N(c2c(N(c3ccc(-c4ccccc4)cc3)c3ccc4ccccc4c3)ccc3ccccc23)c2cccc(-c3ccc4ccccc4c3)c21 ; CC1(C)c2ccccc2N(c2c(N(c3ccc(-c4ccccc4)cc3)c3cccc4ccccc34)ccc3ccccc23)c2cccc(-c3ccc4ccccc4c3)c21 ; CC1(C)c2ccccc2N(c2c(N(c3ccc(-c4ccccc4)cc3)c3ccccc3-c3ccccc3)ccc3ccccc23)c2cccc(-c3ccc4ccccc4c3)c21]

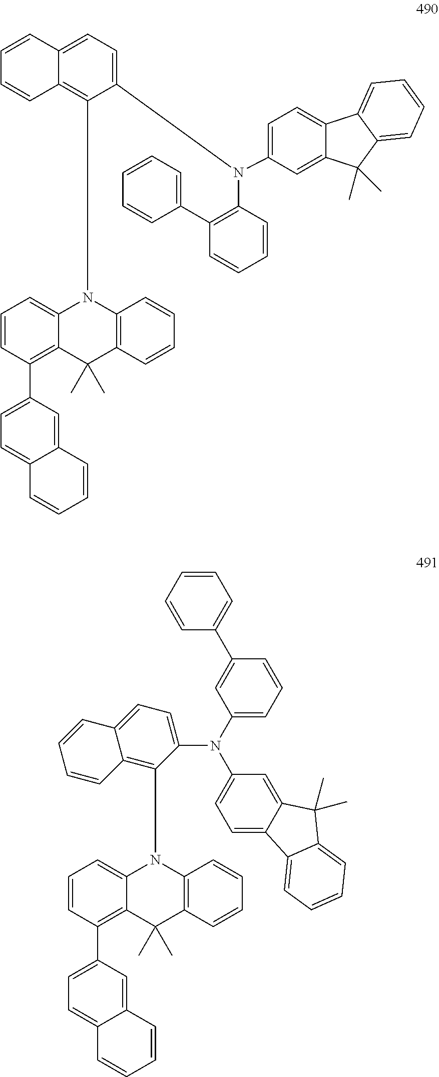 [SMILES: CC1(C)c2ccccc2-c2ccc(N(c3ccccc3-c3ccccc3)c3ccc4ccccc4c3N3c4ccccc4C(C)(C)c4c(-c5ccc6ccccc6c5)cccc43)cc21 ; CC1(C)c2ccccc2-c2ccc(N(c3cccc(-c4ccccc4)c3)c3ccc4ccccc4c3N3c4ccccc4C(C)(C)c4c(-c5ccc6ccccc6c5)cccc43)cc21]

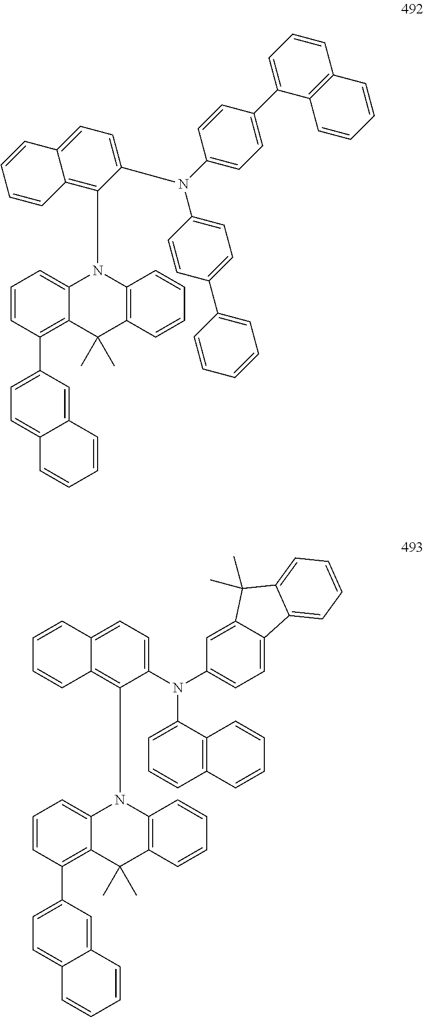 [SMILES: CC1(C)c2ccccc2N(c2c(N(c3ccc(-c4ccccc4)cc3)c3ccc(-c4cccc5ccccc45)cc3)ccc3ccccc23)c2cccc(-c3ccc4ccccc4c3)c21 ; CC1(C)c2ccccc2-c2ccc(N(c3ccc4ccccc4c3N3c4ccccc4C(C)(C)c4c(-c5ccc6ccccc6c5)cccc43)c3cccc4ccccc34)cc21]

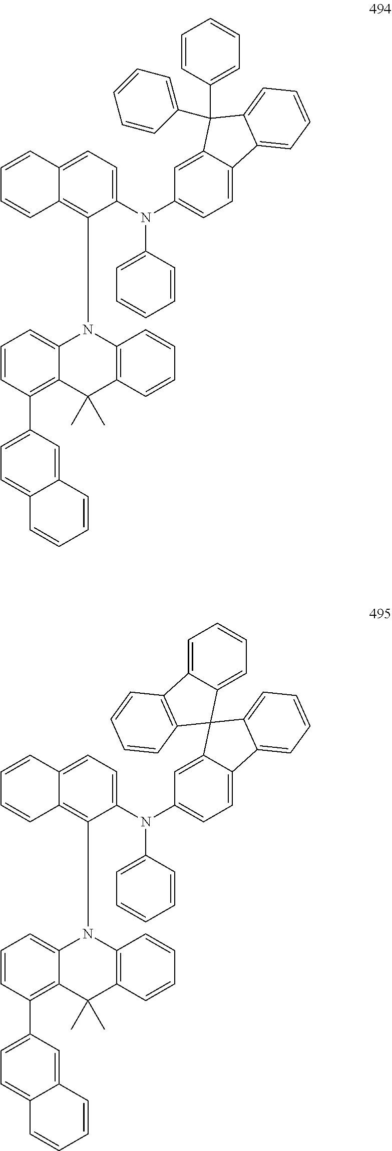 [SMILES: CC1(C)c2ccccc2N(c2c(N(c3ccccc3)c3ccc4c(c3)C(c3ccccc3)(c3ccccc3)c3ccccc3-4)ccc3ccccc23)c2cccc(-c3ccc4ccccc4c3)c21 ; CC1(C)c2ccccc2N(c2c(N(c3ccccc3)c3ccc4c(c3)C3(c5ccccc5-c5ccccc53)c3ccccc3-4)ccc3ccccc23)c2cccc(-c3ccc4ccccc4c3)c21]

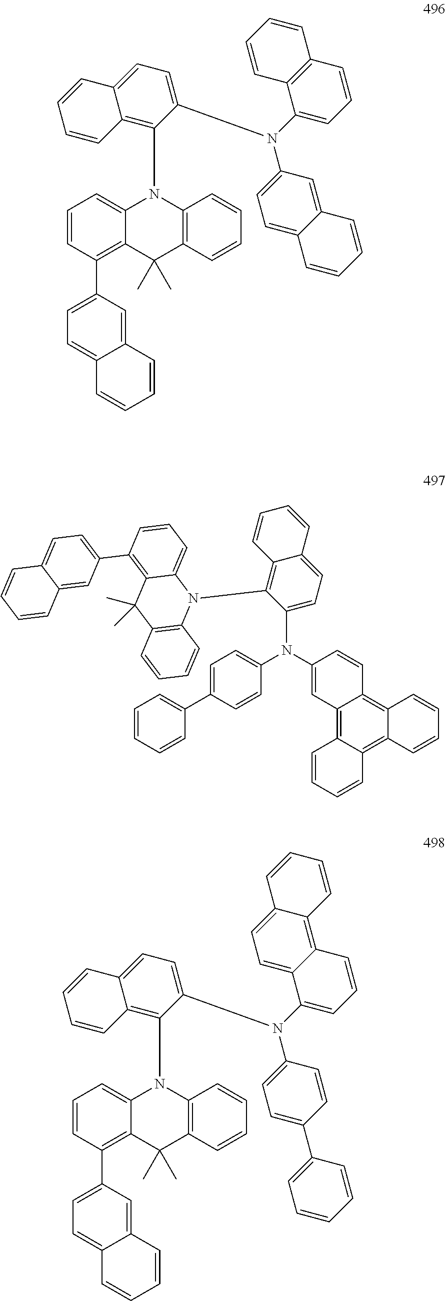 [SMILES: CC1(C)c2ccccc2N(c2c(N(c3ccc4ccccc4c3)c3cccc4ccccc34)ccc3ccccc23)c2cccc(-c3ccc4ccccc4c3)c21 ; CC1(C)c2ccccc2N(c2c(N(c3ccc(-c4ccccc4)cc3)c3ccc4c5ccccc5c5ccccc5c4c3)ccc3ccccc23)c2cccc(-c3ccc4ccccc4c3)c21 ; CC1(C)c2ccccc2N(c2c(N(c3ccc(-c4ccccc4)cc3)c3cccc4c3ccc3ccccc34)ccc3ccccc23)c2cccc(-c3ccc4ccccc4c3)c21]

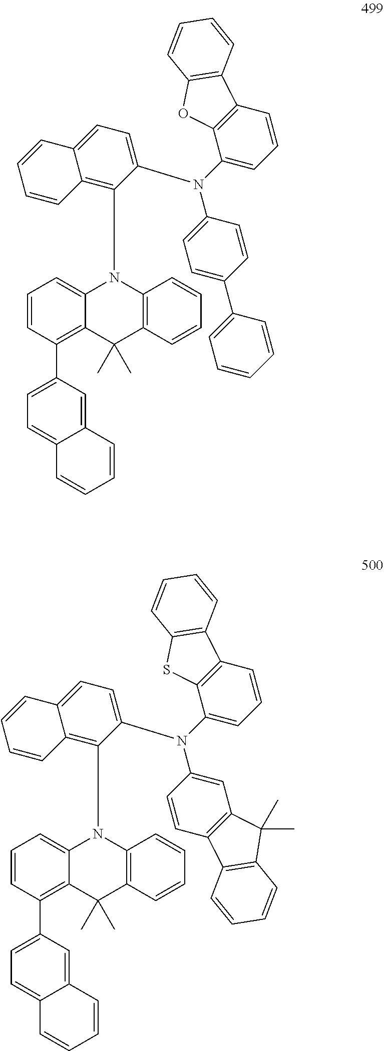 [SMILES: CC1(C)c2ccccc2N(c2c(N(c3ccc(-c4ccccc4)cc3)c3cccc4c3oc3ccccc34)ccc3ccccc23)c2cccc(-c3ccc4ccccc4c3)c21 ; CC1(C)c2ccccc2-c2ccc(N(c3ccc4ccccc4c3N3c4ccccc4C(C)(C)c4c(-c5ccc6ccccc6c5)cccc43)c3cccc4c3sc3ccccc34)cc21]

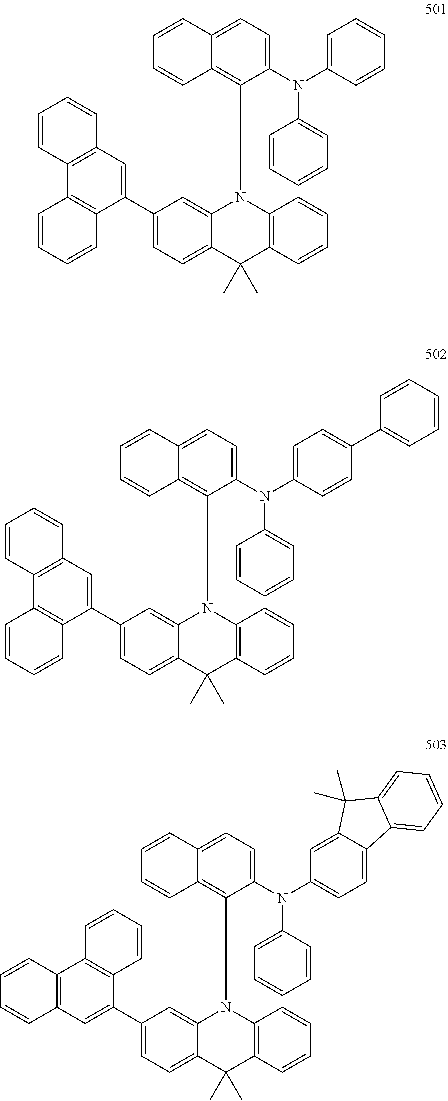 [SMILES: CC1(C)c2ccccc2N(c2c(N(c3ccccc3)c3ccccc3)ccc3ccccc23)c2cc(-c3cc4ccccc4c4ccccc34)ccc21 ; CC1(C)c2ccccc2N(c2c(N(c3ccccc3)c3ccc(-c4ccccc4)cc3)ccc3ccccc23)c2cc(-c3cc4ccccc4c4ccccc34)ccc21 ; CC1(C)c2ccccc2-c2ccc(N(c3ccccc3)c3ccc4ccccc4c3N3c4ccccc4C(C)(C)c4ccc(-c5cc6ccccc6c6ccccc56)cc43)cc21]

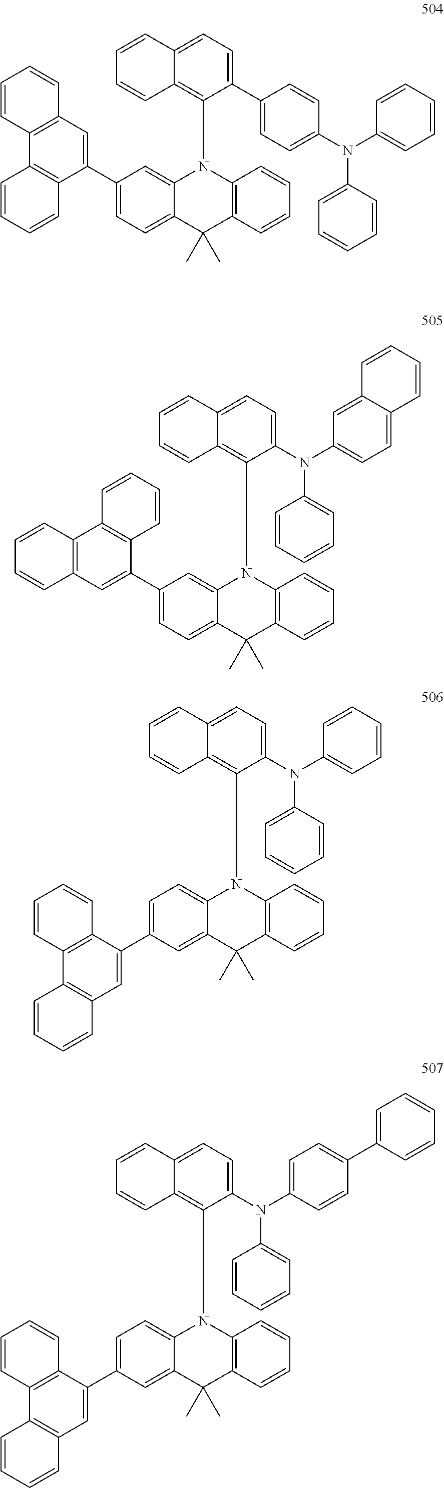

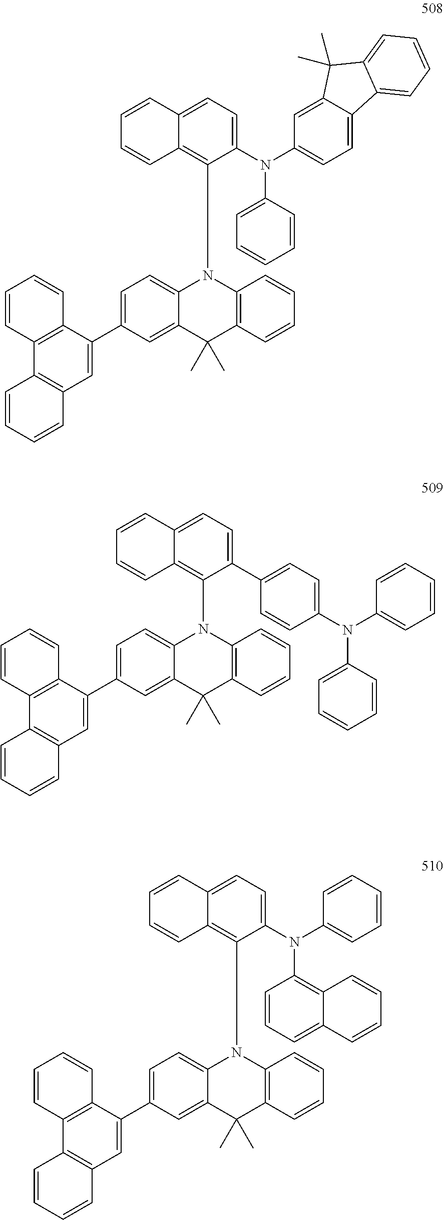 [SMILES: CC1(C)c2ccccc2-c2ccc(N(c3ccccc3)c3ccc4ccccc4c3N3c4ccccc4C(C)(C)c4cc(-c5cc6ccccc6c6ccccc56)ccc43)cc21 ; CC1(C)c2ccccc2N(c2c(-c3ccc(N(c4ccccc4)c4ccccc4)cc3)ccc3ccccc23)c2ccc(-c3cc4ccccc4c4ccccc34)cc21 ; CC1(C)c2ccccc2N(c2c(N(c3ccccc3)c3cccc4ccccc34)ccc3ccccc23)c2ccc(-c3cc4ccccc4c4ccccc34)cc21]

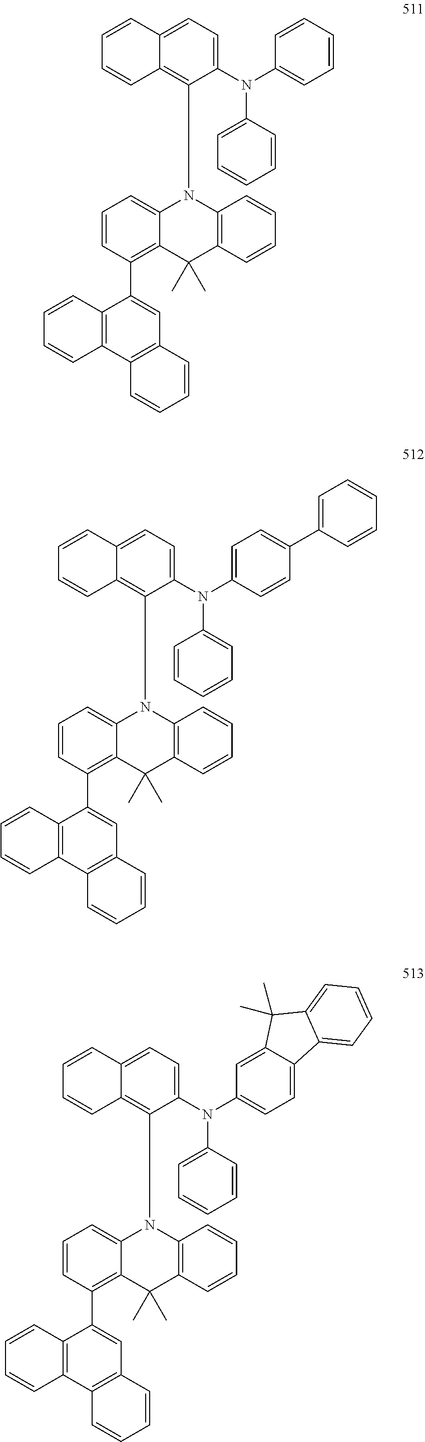 [SMILES: CC1(C)c2ccccc2N(c2c(N(c3ccccc3)c3ccccc3)ccc3ccccc23)c2cccc(-c3cc4ccccc4c4ccccc34)c21 ; CC1(C)c2ccccc2N(c2c(N(c3ccccc3)c3ccc(-c4ccccc4)cc3)ccc3ccccc23)c2cccc(-c3cc4ccccc4c4ccccc34)c21 ; CC1(C)c2ccccc2-c2ccc(N(c3ccccc3)c3ccc4ccccc4c3N3c4ccccc4C(C)(C)c4c(-c5cc6ccccc6c6ccccc56)cccc43)cc21]

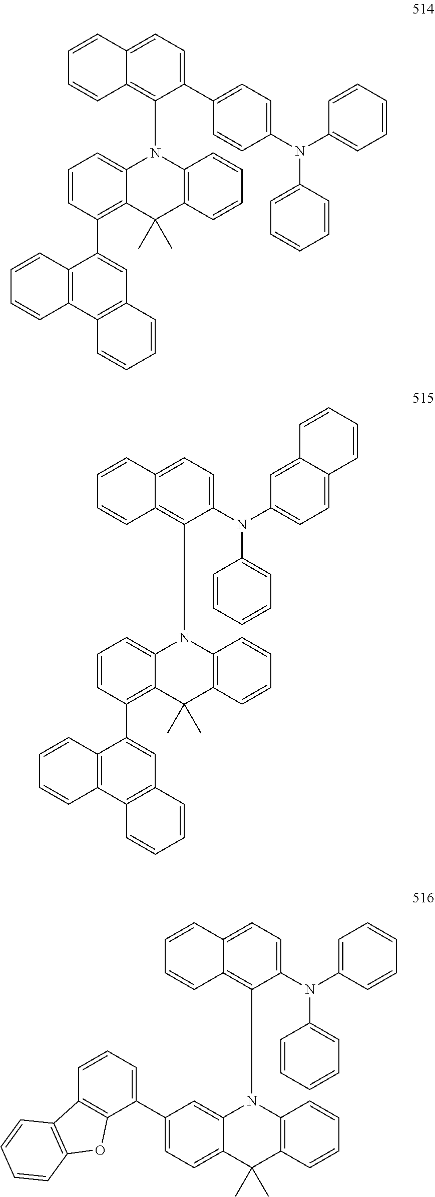 [SMILES: CC1(C)c2ccccc2N(c2c(-c3ccc(N(c4ccccc4)c4ccccc4)cc3)ccc3ccccc23)c2cccc(-c3cc4ccccc4c4ccccc34)c21 ; CC1(C)c2ccccc2N(c2c(N(c3ccccc3)c3ccc4ccccc4c3)ccc3ccccc23)c2cccc(-c3cc4ccccc4c4ccccc34)c21 ; CC1(C)c2ccccc2N(c2c(N(c3ccccc3)c3ccccc3)ccc3ccccc23)c2cc(-c3cccc4c3oc3ccccc34)ccc21]

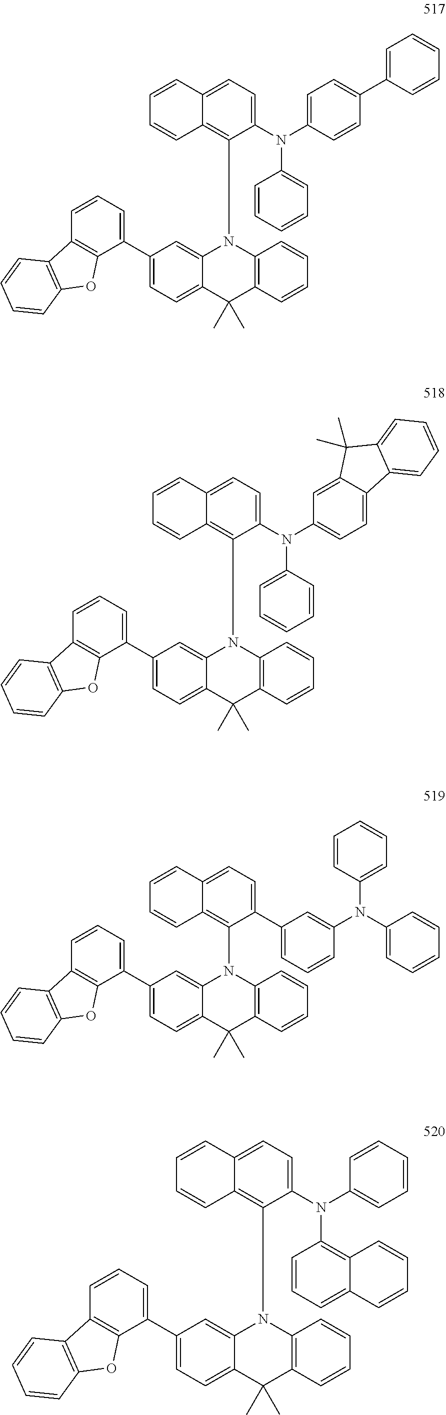 [SMILES: CC1(C)c2ccccc2N(c2c(N(c3ccccc3)c3ccc(-c4ccccc4)cc3)ccc3ccccc23)c2cc(-c3cccc4c3oc3ccccc34)ccc21 ; CC1(C)c2ccccc2-c2ccc(N(c3ccccc3)c3ccc4ccccc4c3N3c4ccccc4C(C)(C)c4ccc(-c5cccc6c5oc5ccccc56)cc43)cc21 ; CC1(C)c2ccccc2N(c2c(-c3cccc(N(c4ccccc4)c4ccccc4)c3)ccc3ccccc23)c2cc(-c3cccc4c3oc3ccccc34)ccc21 ; CC1(C)c2ccccc2N(c2c(N(c3ccccc3)c3cccc4ccccc34)ccc3ccccc23)c2cc(-c3cccc4c3oc3ccccc34)ccc21]

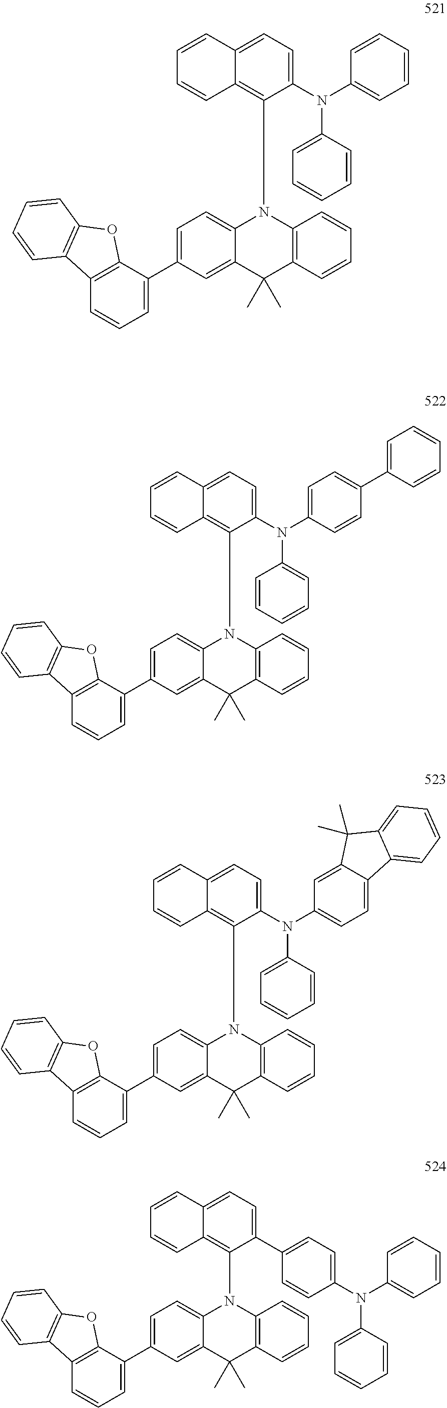 [SMILES: CC1(C)c2ccccc2N(c2c(N(c3ccccc3)c3ccccc3)ccc3ccccc23)c2ccc(-c3cccc4c3oc3ccccc34)cc21 ; CC1(C)c2ccccc2N(c2c(N(c3ccccc3)c3ccc(-c4ccccc4)cc3)ccc3ccccc23)c2ccc(-c3cccc4c3oc3ccccc34)cc21 ; CC1(C)c2ccccc2-c2ccc(N(c3ccccc3)c3ccc4ccccc4c3N3c4ccccc4C(C)(C)c4cc(-c5cccc6c5oc5ccccc56)ccc43)cc21 ; CC1(C)c2ccccc2N(c2c(-c3ccc(N(c4ccccc4)c4ccccc4)cc3)ccc3ccccc23)c2ccc(-c3cccc4c3oc3ccccc34)cc21]

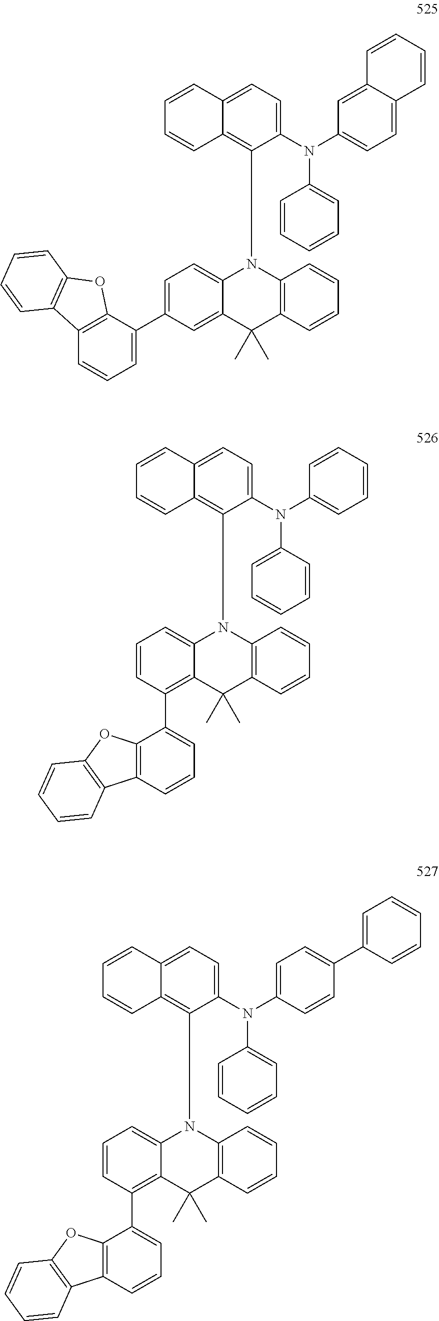 [SMILES: CC1(C)c2ccccc2N(c2c(N(c3ccccc3)c3ccc4ccccc4c3)ccc3ccccc23)c2ccc(-c3cccc4c3oc3ccccc34)cc21 ; CC1(C)c2ccccc2N(c2c(N(c3ccccc3)c3ccccc3)ccc3ccccc23)c2cccc(-c3cccc4c3oc3ccccc34)c21 ; CC1(C)c2ccccc2N(c2c(N(c3ccccc3)c3ccc(-c4ccccc4)cc3)ccc3ccccc23)c2cccc(-c3cccc4c3oc3ccccc34)c21]

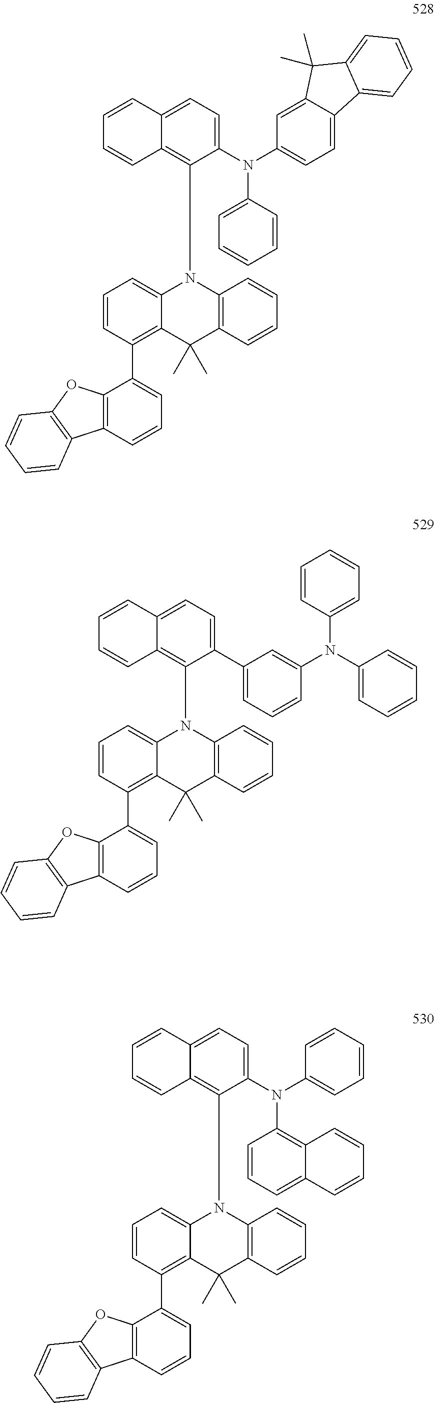 [SMILES: CC1(C)c2ccccc2-c2ccc(N(c3ccccc3)c3ccc4ccccc4c3N3c4ccccc4C(C)(C)c4c(-c5cccc6c5oc5ccccc56)cccc43)cc21 ; CC1(C)c2ccccc2N(c2c(-c3cccc(N(c4ccccc4)c4ccccc4)c3)ccc3ccccc23)c2cccc(-c3cccc4c3oc3ccccc34)c21 ; CC1(C)c2ccccc2N(c2c(N(c3ccccc3)c3cccc4ccccc34)ccc3ccccc23)c2cccc(-c3cccc4c3oc3ccccc34)c21]

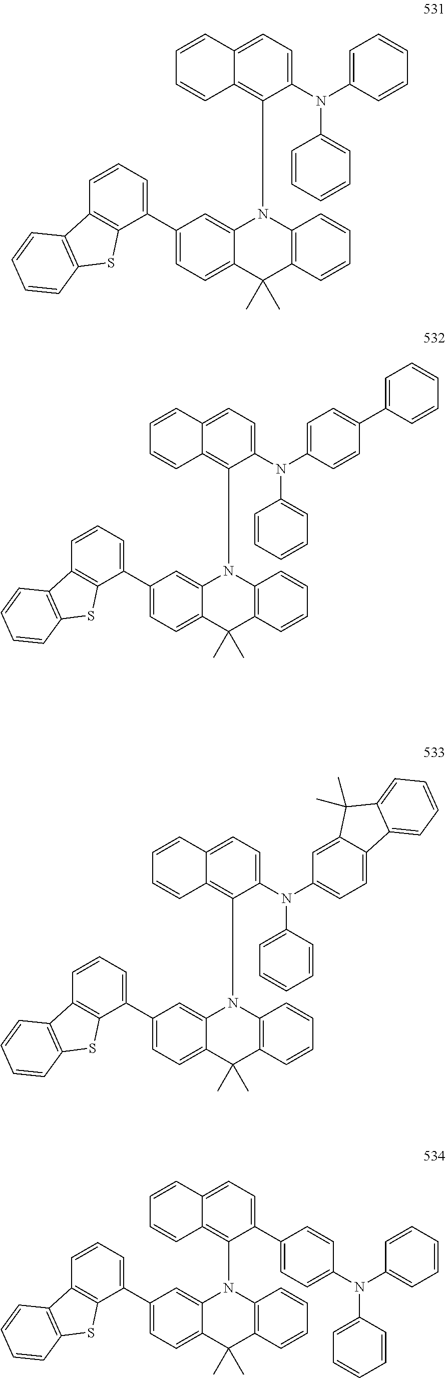 [SMILES: CC1(C)c2ccccc2N(c2c(N(c3ccccc3)c3ccccc3)ccc3ccccc23)c2cc(-c3cccc4c3sc3ccccc34)ccc21 ; CC1(C)c2ccccc2N(c2c(N(c3ccccc3)c3ccc(-c4ccccc4)cc3)ccc3ccccc23)c2cc(-c3cccc4c3sc3ccccc34)ccc21 ; CC1(C)c2ccccc2-c2ccc(N(c3ccccc3)c3ccc4ccccc4c3N3c4ccccc4C(C)(C)c4ccc(-c5cccc6c5sc5ccccc56)cc43)cc21 ; CC1(C)c2ccccc2N(c2c(-c3ccc(N(c4ccccc4)c4ccccc4)cc3)ccc3ccccc23)c2cc(-c3cccc4c3sc3ccccc34)ccc21]

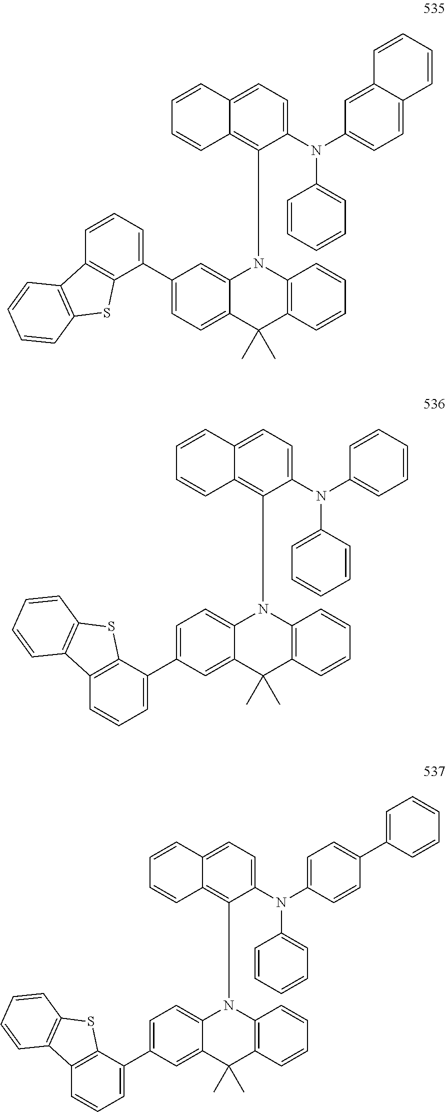 [SMILES: CC1(C)c2ccccc2N(c2c(N(c3ccccc3)c3ccc4ccccc4c3)ccc3ccccc23)c2cc(-c3cccc4c3sc3ccccc34)ccc21 ; CC1(C)c2ccccc2N(c2c(N(c3ccccc3)c3ccccc3)ccc3ccccc23)c2ccc(-c3cccc4c3sc3ccccc34)cc21 ; CC1(C)c2ccccc2N(c2c(N(c3ccccc3)c3ccc(-c4ccccc4)cc3)ccc3ccccc23)c2ccc(-c3cccc4c3sc3ccccc34)cc21]

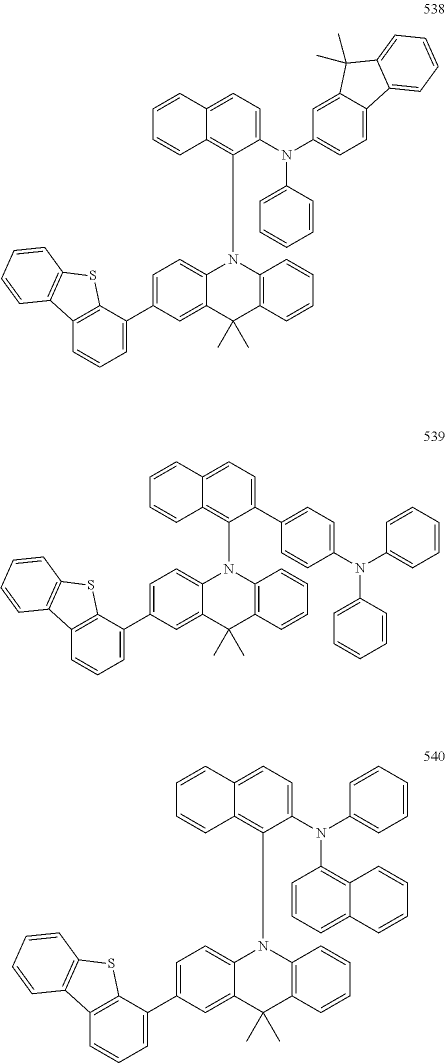 [SMILES: CC1(C)c2ccccc2-c2ccc(N(c3ccccc3)c3ccc4ccccc4c3N3c4ccccc4C(C)(C)c4cc(-c5cccc6c5sc5ccccc56)ccc43)cc21 ; CC1(C)c2ccccc2N(c2c(-c3ccc(N(c4ccccc4)c4ccccc4)cc3)ccc3ccccc23)c2ccc(-c3cccc4c3sc3ccccc34)cc21 ; CC1(C)c2ccccc2N(c2c(N(c3ccccc3)c3cccc4ccccc34)ccc3ccccc23)c2ccc(-c3cccc4c3sc3ccccc34)cc21]

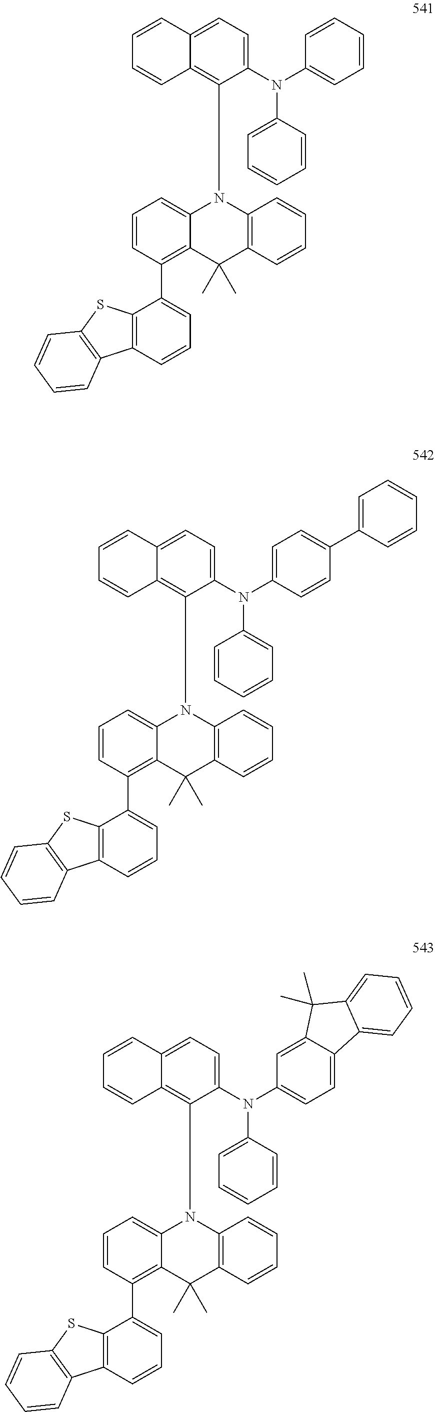 [SMILES: CC1(C)c2ccccc2N(c2c(N(c3ccccc3)c3ccccc3)ccc3ccccc23)c2cccc(-c3cccc4c3sc3ccccc34)c21 ; CC1(C)c2ccccc2N(c2c(N(c3ccccc3)c3ccc(-c4ccccc4)cc3)ccc3ccccc23)c2cccc(-c3cccc4c3sc3ccccc34)c21 ; CC1(C)c2ccccc2-c2ccc(N(c3ccccc3)c3ccc4ccccc4c3N3c4ccccc4C(C)(C)c4c(-c5cccc6c5sc5ccccc56)cccc43)cc21]

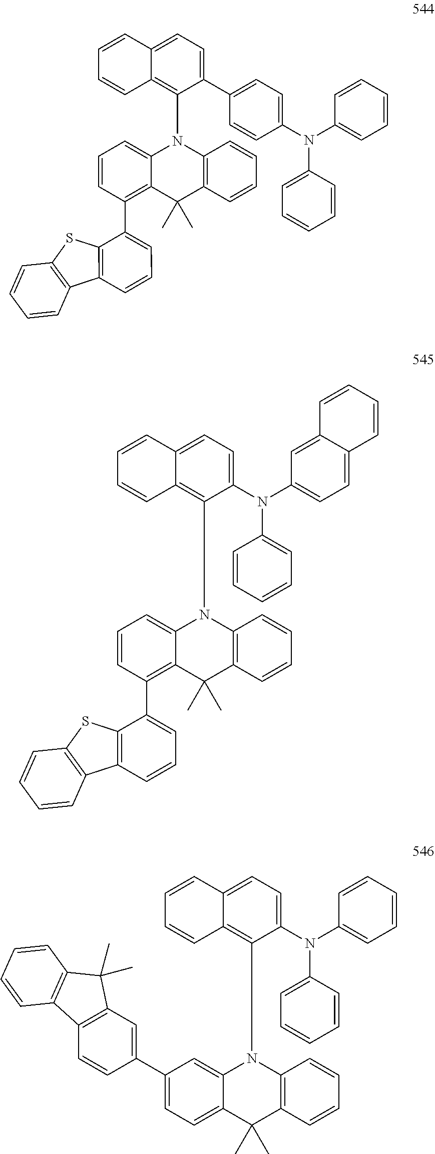 [SMILES: CC1(C)c2ccccc2N(c2c(-c3ccc(N(c4ccccc4)c4ccccc4)cc3)ccc3ccccc23)c2cccc(-c3cccc4c3sc3ccccc34)c21 ; CC1(C)c2ccccc2N(c2c(N(c3ccccc3)c3ccc4ccccc4c3)ccc3ccccc23)c2cccc(-c3cccc4c3sc3ccccc34)c21 ; CC1(C)c2ccccc2-c2ccc(-c3ccc4c(c3)N(c3c(N(c5ccccc5)c5ccccc5)ccc5ccccc35)c3ccccc3C4(C)C)cc21]

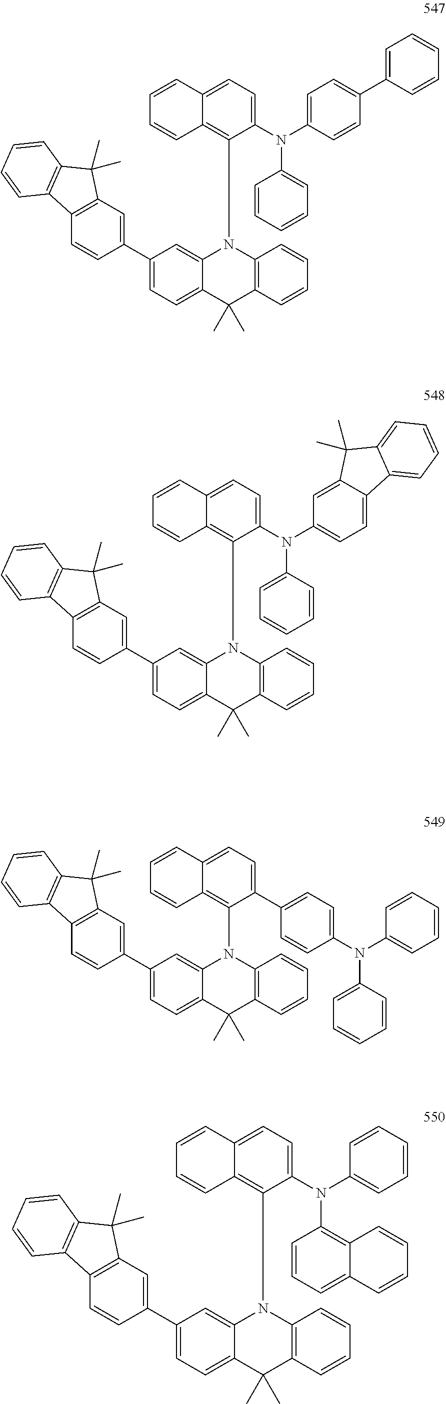 [SMILES: CC1(C)c2ccccc2-c2ccc(-c3ccc4c(c3)N(c3c(N(c5ccccc5)c5ccc(-c6ccccc6)cc5)ccc5ccccc35)c3ccccc3C4(C)C)cc21 ; CC1(C)c2ccccc2-c2ccc(-c3ccc4c(c3)N(c3c(N(c5ccccc5)c5ccc6c(c5)C(C)(C)c5ccccc5-6)ccc5ccccc35)c3ccccc3C4(C)C)cc21 ; CC1(C)c2ccccc2-c2ccc(-c3ccc4c(c3)N(c3c(-c5ccc(N(c6ccccc6)c6ccccc6)cc5)ccc5ccccc35)c3ccccc3C4(C)C)cc21 ; CC1(C)c2ccccc2-c2ccc(-c3ccc4c(c3)N(c3c(N(c5ccccc5)c5cccc6ccccc56)ccc5ccccc35)c3ccccc3C4(C)C)cc21]

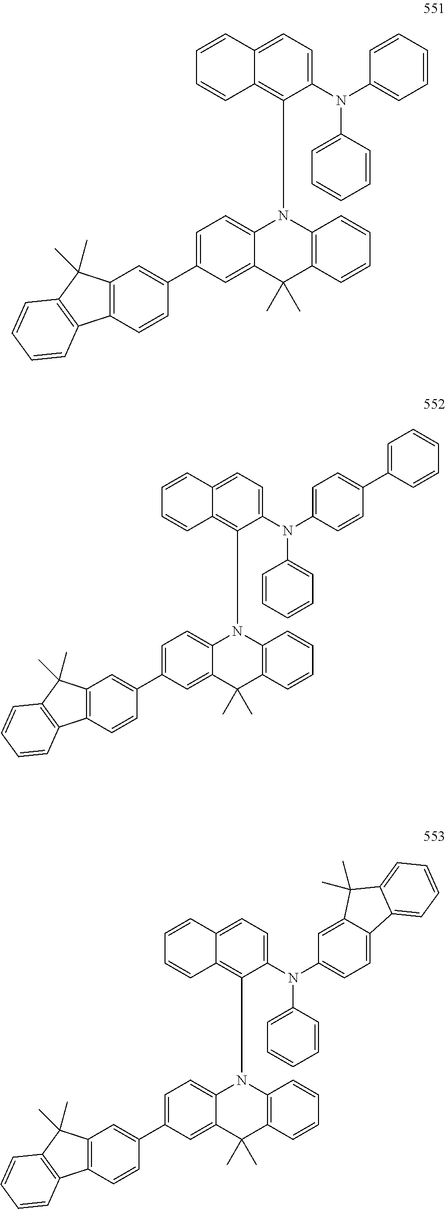 [SMILES: CC1(C)c2ccccc2-c2ccc(-c3ccc4c(c3)C(C)(C)c3ccccc3N4c3c(N(c4ccccc4)c4ccccc4)ccc4ccccc34)cc21 ; CC1(C)c2ccccc2-c2ccc(-c3ccc4c(c3)C(C)(C)c3ccccc3N4c3c(N(c4ccccc4)c4ccc(-c5ccccc5)cc4)ccc4ccccc34)cc21 ; CC1(C)c2ccccc2-c2ccc(-c3ccc4c(c3)C(C)(C)c3ccccc3N4c3c(N(c4ccccc4)c4ccc5c(c4)C(C)(C)c4ccccc4-5)ccc4ccccc34)cc21]

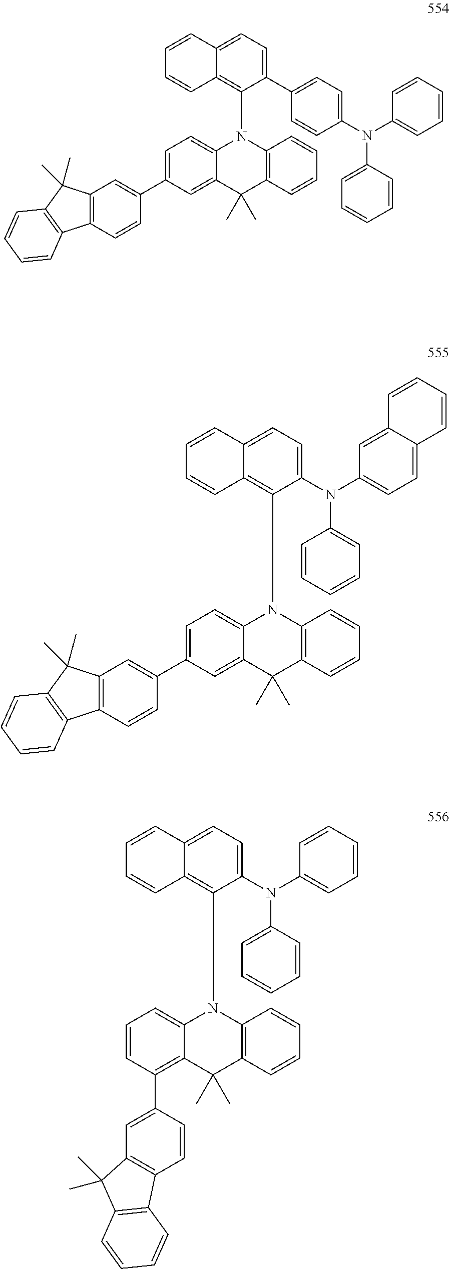 [SMILES: CC1(C)c2ccccc2-c2ccc(-c3ccc4c(c3)C(C)(C)c3ccccc3N4c3c(-c4ccc(N(c5ccccc5)c5ccccc5)cc4)ccc4ccccc34)cc21 ; CC1(C)c2ccccc2-c2ccc(-c3ccc4c(c3)C(C)(C)c3ccccc3N4c3c(N(c4ccccc4)c4ccc5ccccc5c4)ccc4ccccc34)cc21 ; CC1(C)c2ccccc2-c2ccc(-c3cccc4c3C(C)(C)c3ccccc3N4c3c(N(c4ccccc4)c4ccccc4)ccc4ccccc34)cc21]

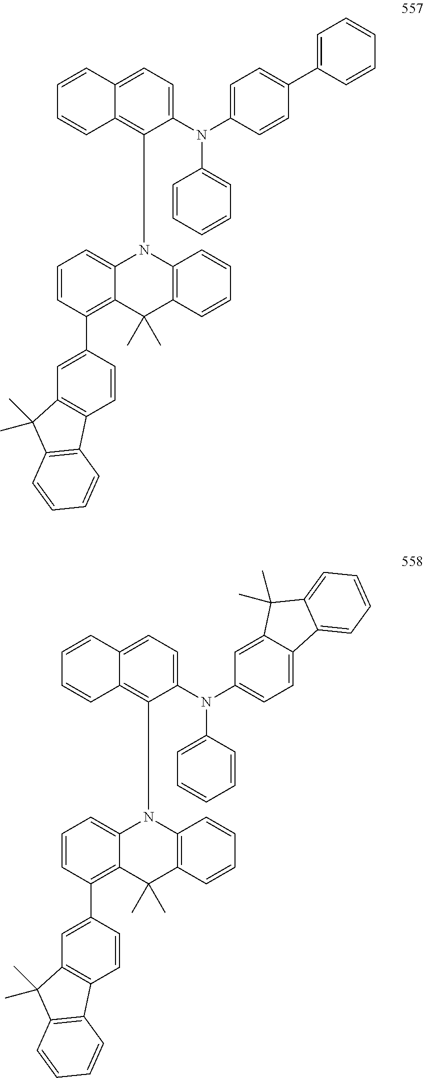 [SMILES: CC1(C)c2ccccc2-c2ccc(-c3cccc4c3C(C)(C)c3ccccc3N4c3c(N(c4ccccc4)c4ccc(-c5ccccc5)cc4)ccc4ccccc34)cc21 ; CC1(C)c2ccccc2-c2ccc(-c3cccc4c3C(C)(C)c3ccccc3N4c3c(N(c4ccccc4)c4ccc5c(c4)C(C)(C)c4ccccc4-5)ccc4ccccc34)cc21]

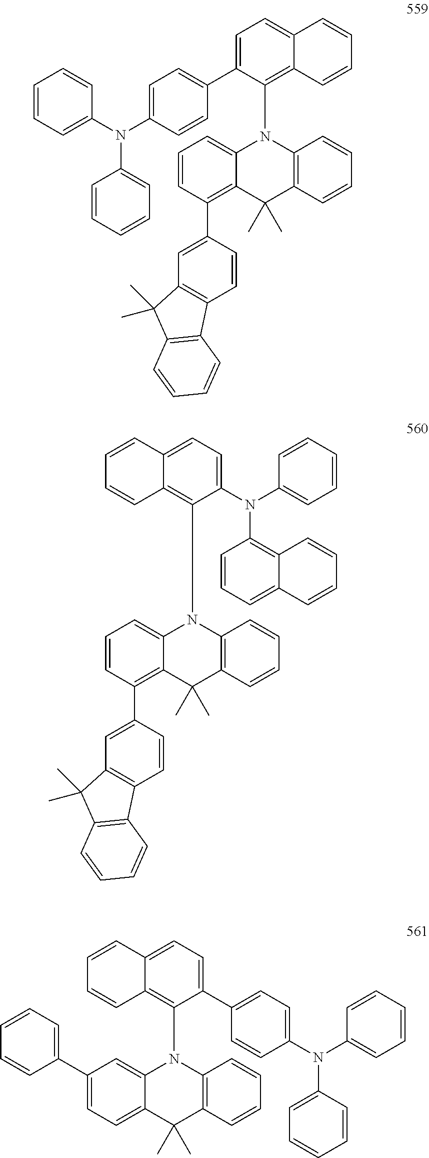

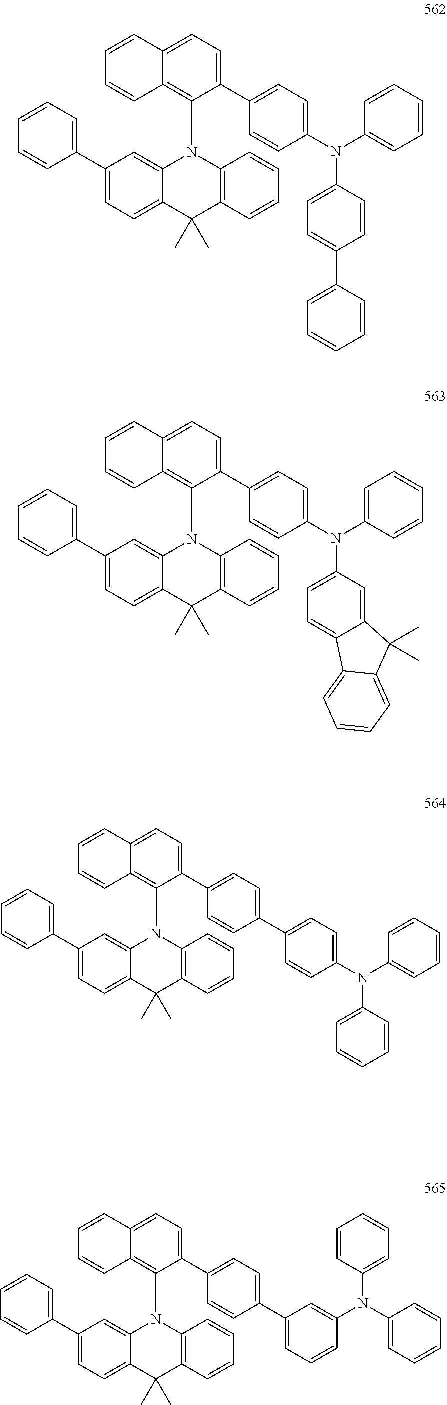 [SMILES: CC1(C)c2ccccc2N(c2c(-c3ccc(N(c4ccccc4)c4ccc(-c5ccccc5)cc4)cc3)ccc3ccccc23)c2cc(-c3ccccc3)ccc21 ; CC1(C)c2ccccc2-c2ccc(N(c3ccccc3)c3ccc(-c4ccc5ccccc5c4N4c5ccccc5C(C)(C)c5ccc(-c6ccccc6)cc54)cc3)cc21 ; CC1(C)c2ccccc2N(c2c(-c3ccc(-c4ccc(N(c5ccccc5)c5ccccc5)cc4)cc3)ccc3ccccc23)c2cc(-c3ccccc3)ccc21 ; CC1(C)c2ccccc2N(c2c(-c3ccc(-c4cccc(N(c5ccccc5)c5ccccc5)c4)cc3)ccc3ccccc23)c2cc(-c3ccccc3)ccc21]

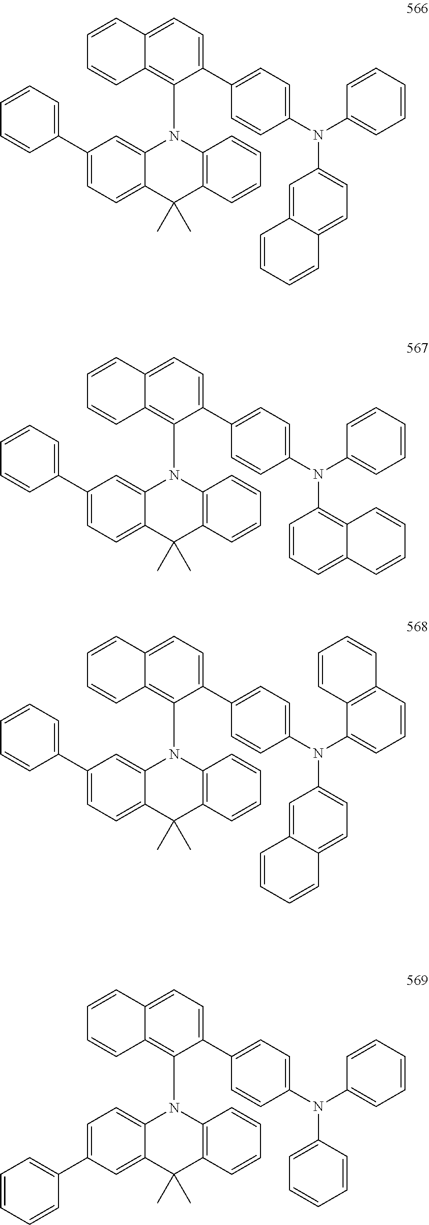 [SMILES: CC1(C)c2ccccc2N(c2c(-c3ccc(N(c4ccccc4)c4ccc5ccccc5c4)cc3)ccc3ccccc23)c2cc(-c3ccccc3)ccc21 ; CC1(C)c2ccccc2N(c2c(-c3ccc(N(c4ccccc4)c4cccc5ccccc45)cc3)ccc3ccccc23)c2cc(-c3ccccc3)ccc21 ; CC1(C)c2ccccc2N(c2c(-c3ccc(N(c4ccc5ccccc5c4)c4cccc5ccccc45)cc3)ccc3ccccc23)c2cc(-c3ccccc3)ccc21 ; CC1(C)c2ccccc2N(c2c(-c3ccc(N(c4ccccc4)c4ccccc4)cc3)ccc3ccccc23)c2ccc(-c3ccccc3)cc21]

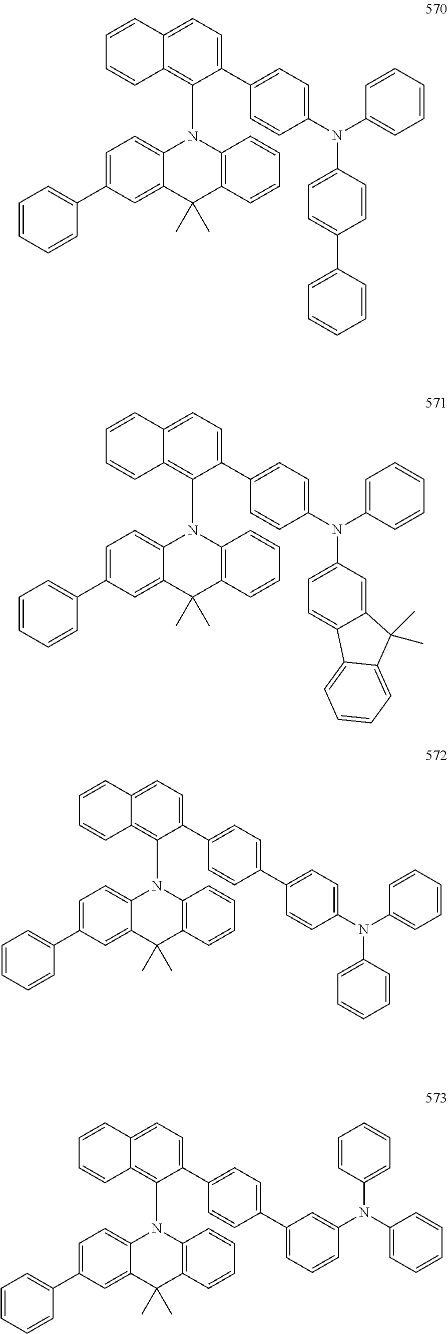 [SMILES: CC1(C)c2ccccc2N(c2c(-c3ccc(N(c4ccccc4)c4ccc(-c5ccccc5)cc4)cc3)ccc3ccccc23)c2ccc(-c3ccccc3)cc21 ; CC1(C)c2ccccc2-c2ccc(N(c3ccccc3)c3ccc(-c4ccc5ccccc5c4N4c5ccccc5C(C)(C)c5cc(-c6ccccc6)ccc54)cc3)cc21 ; CC1(C)c2ccccc2N(c2c(-c3ccc(-c4ccc(N(c5ccccc5)c5ccccc5)cc4)cc3)ccc3ccccc23)c2ccc(-c3ccccc3)cc21 ; CC1(C)c2ccccc2N(c2c(-c3ccc(-c4cccc(N(c5ccccc5)c5ccccc5)c4)cc3)ccc3ccccc23)c2ccc(-c3ccccc3)cc21]

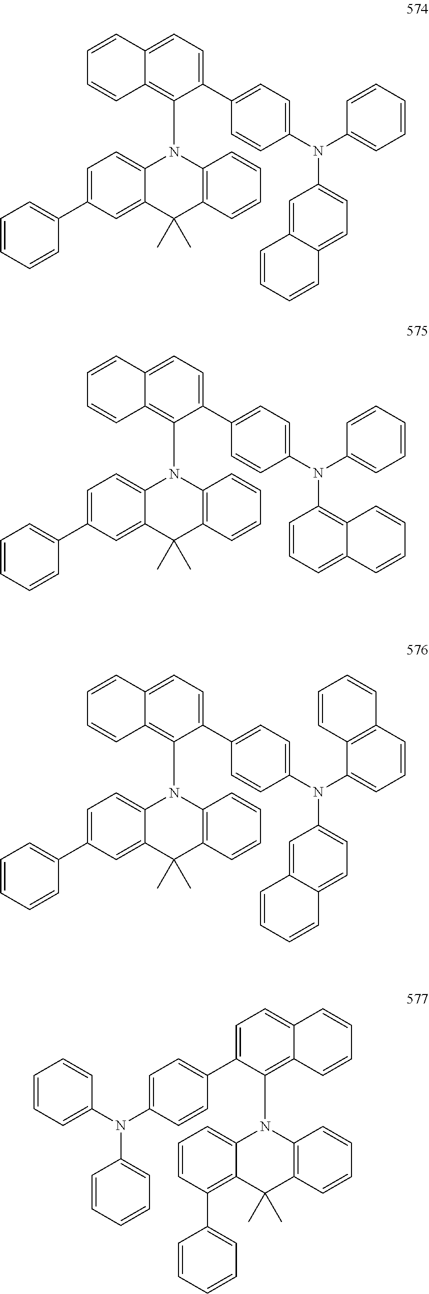 [SMILES: CC1(C)c2ccccc2N(c2c(-c3ccc(N(c4ccccc4)c4ccc5ccccc5c4)cc3)ccc3ccccc23)c2ccc(-c3ccccc3)cc21 ; CC1(C)c2ccccc2N(c2c(-c3ccc(N(c4ccccc4)c4cccc5ccccc45)cc3)ccc3ccccc23)c2ccc(-c3ccccc3)cc21 ; CC1(C)c2ccccc2N(c2c(-c3ccc(N(c4ccc5ccccc5c4)c4cccc5ccccc45)cc3)ccc3ccccc23)c2ccc(-c3ccccc3)cc21 ; CC1(C)c2ccccc2N(c2c(-c3ccc(N(c4ccccc4)c4ccccc4)cc3)ccc3ccccc23)c2cccc(-c3ccccc3)c21]

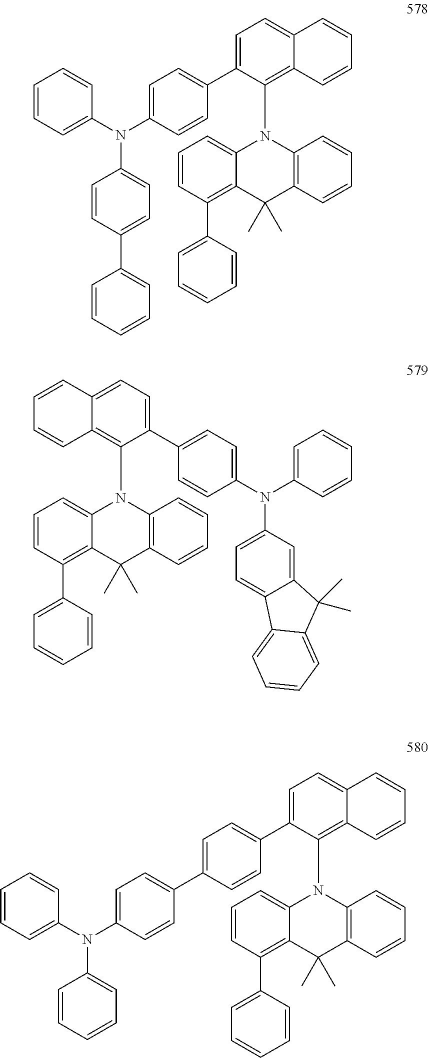

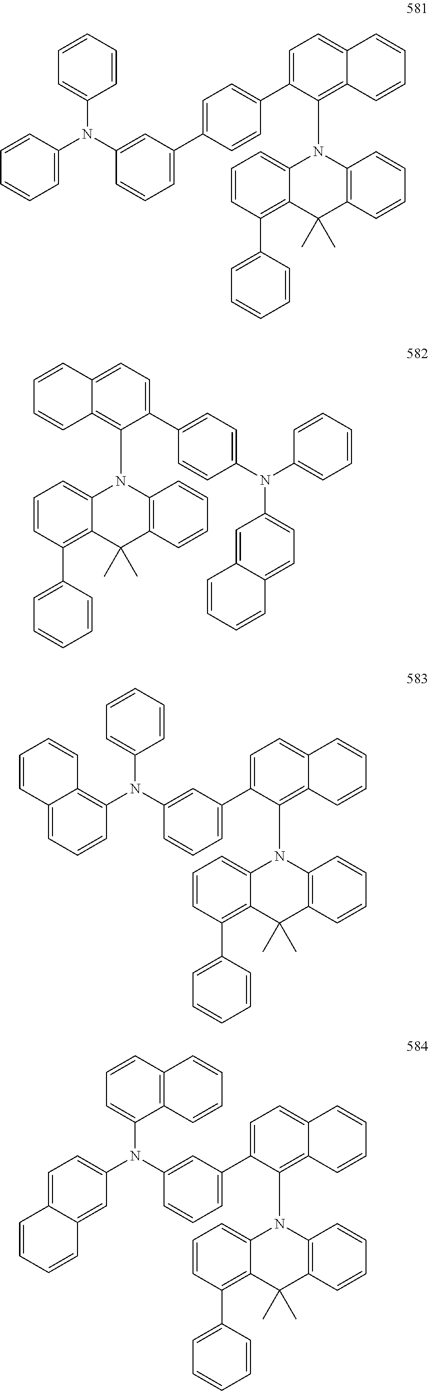

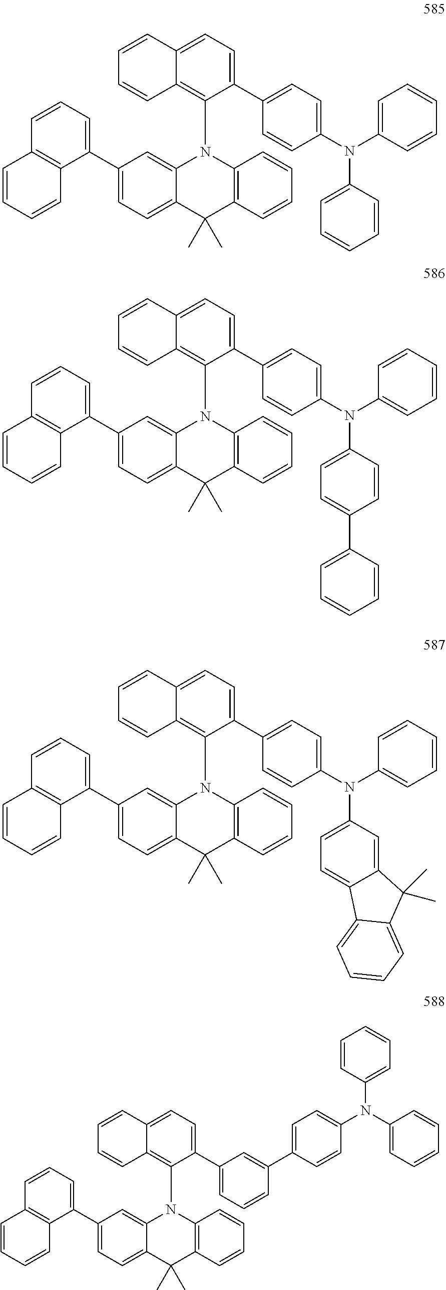 [SMILES: CC1(C)c2ccccc2N(c2c(-c3ccc(N(c4ccccc4)c4ccccc4)cc3)ccc3ccccc23)c2cc(-c3cccc4ccccc34)ccc21 ; CC1(C)c2ccccc2N(c2c(-c3ccc(N(c4ccccc4)c4ccc(-c5ccccc5)cc4)cc3)ccc3ccccc23)c2cc(-c3cccc4ccccc34)ccc21 ; CC1(C)c2ccccc2-c2ccc(N(c3ccccc3)c3ccc(-c4ccc5ccccc5c4N4c5ccccc5C(C)(C)c5ccc(-c6cccc7ccccc67)cc54)cc3)cc21 ; CC1(C)c2ccccc2N(c2c(-c3cccc(-c4ccc(N(c5ccccc5)c5ccccc5)cc4)c3)ccc3ccccc23)c2cc(-c3cccc4ccccc34)ccc21]

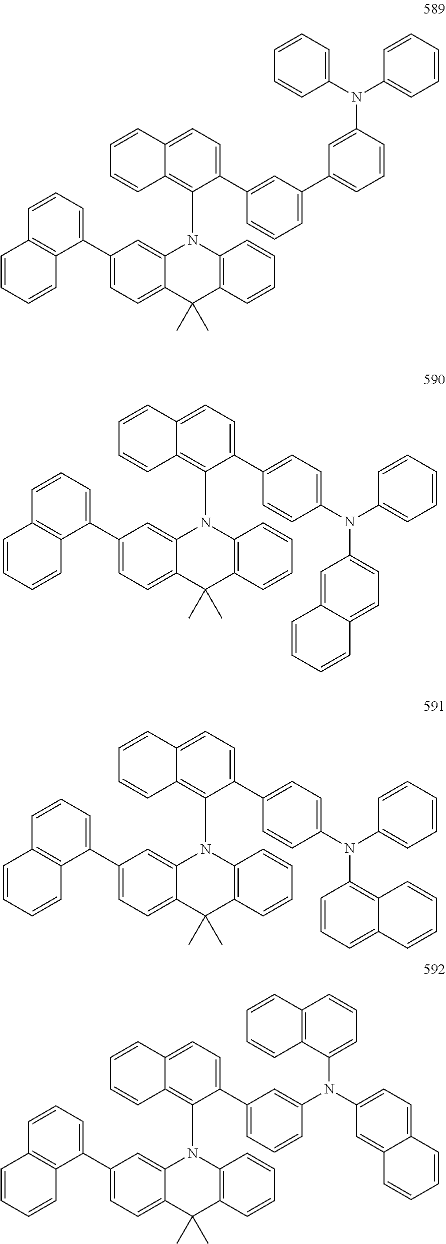 [SMILES: CC1(C)c2ccccc2N(c2c(-c3cccc(-c4cccc(N(c5ccccc5)c5ccccc5)c4)c3)ccc3ccccc23)c2cc(-c3cccc4ccccc34)ccc21 ; CC1(C)c2ccccc2N(c2c(-c3ccc(N(c4ccccc4)c4ccc5ccccc5c4)cc3)ccc3ccccc23)c2cc(-c3cccc4ccccc34)ccc21 ; CC1(C)c2ccccc2N(c2c(-c3ccc(N(c4ccccc4)c4cccc5ccccc45)cc3)ccc3ccccc23)c2cc(-c3cccc4ccccc34)ccc21 ; CC1(C)c2ccccc2N(c2c(-c3cccc(N(c4ccc5ccccc5c4)c4cccc5ccccc45)c3)ccc3ccccc23)c2cc(-c3cccc4ccccc34)ccc21]

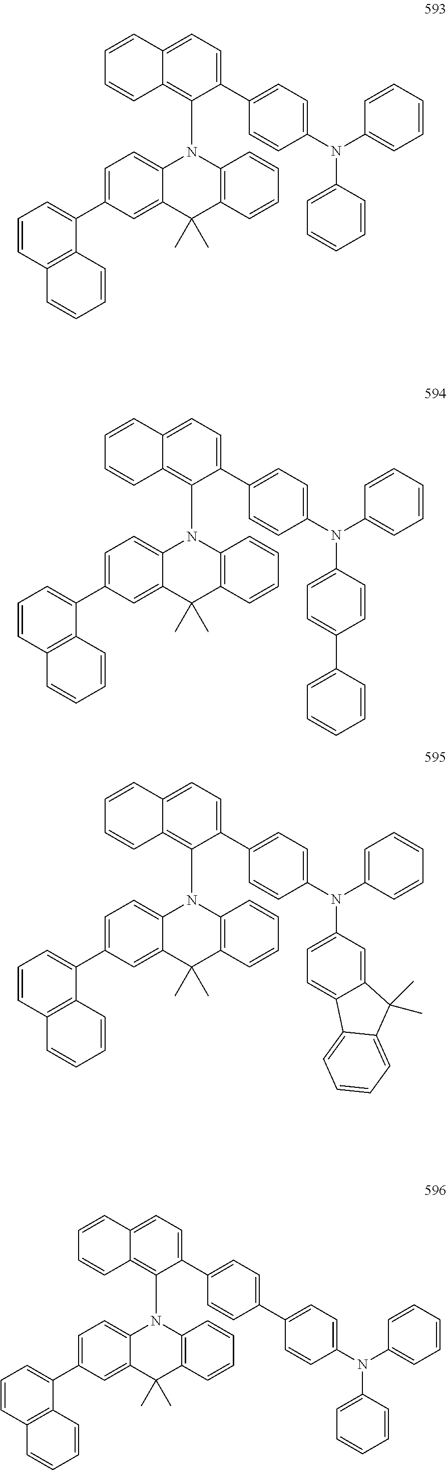 [SMILES: CC1(C)c2ccccc2N(c2c(-c3ccc(N(c4ccccc4)c4ccccc4)cc3)ccc3ccccc23)c2ccc(-c3cccc4ccccc34)cc21 ; CC1(C)c2ccccc2N(c2c(-c3ccc(N(c4ccccc4)c4ccc(-c5ccccc5)cc4)cc3)ccc3ccccc23)c2ccc(-c3cccc4ccccc34)cc21 ; CC1(C)c2ccccc2-c2ccc(N(c3ccccc3)c3ccc(-c4ccc5ccccc5c4N4c5ccccc5C(C)(C)c5cc(-c6cccc7ccccc67)ccc54)cc3)cc21 ; CC1(C)c2ccccc2N(c2c(-c3ccc(-c4ccc(N(c5ccccc5)c5ccccc5)cc4)cc3)ccc3ccccc23)c2ccc(-c3cccc4ccccc34)cc21]

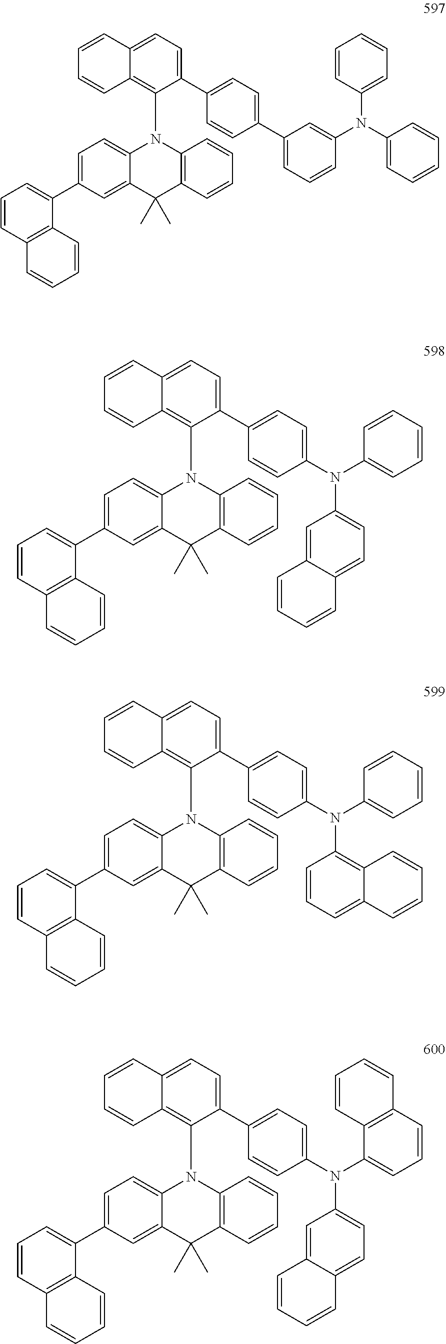 [SMILES: CC1(C)c2ccccc2N(c2c(-c3ccc(-c4cccc(N(c5ccccc5)c5ccccc5)c4)cc3)ccc3ccccc23)c2ccc(-c3cccc4ccccc34)cc21 ; CC1(C)c2ccccc2N(c2c(-c3ccc(N(c4ccccc4)c4ccc5ccccc5c4)cc3)ccc3ccccc23)c2ccc(-c3cccc4ccccc34)cc21 ; CC1(C)c2ccccc2N(c2c(-c3ccc(N(c4ccccc4)c4cccc5ccccc45)cc3)ccc3ccccc23)c2ccc(-c3cccc4ccccc34)cc21 ; CC1(C)c2ccccc2N(c2c(-c3ccc(N(c4ccc5ccccc5c4)c4cccc5ccccc45)cc3)ccc3ccccc23)c2ccc(-c3cccc4ccccc34)cc21]

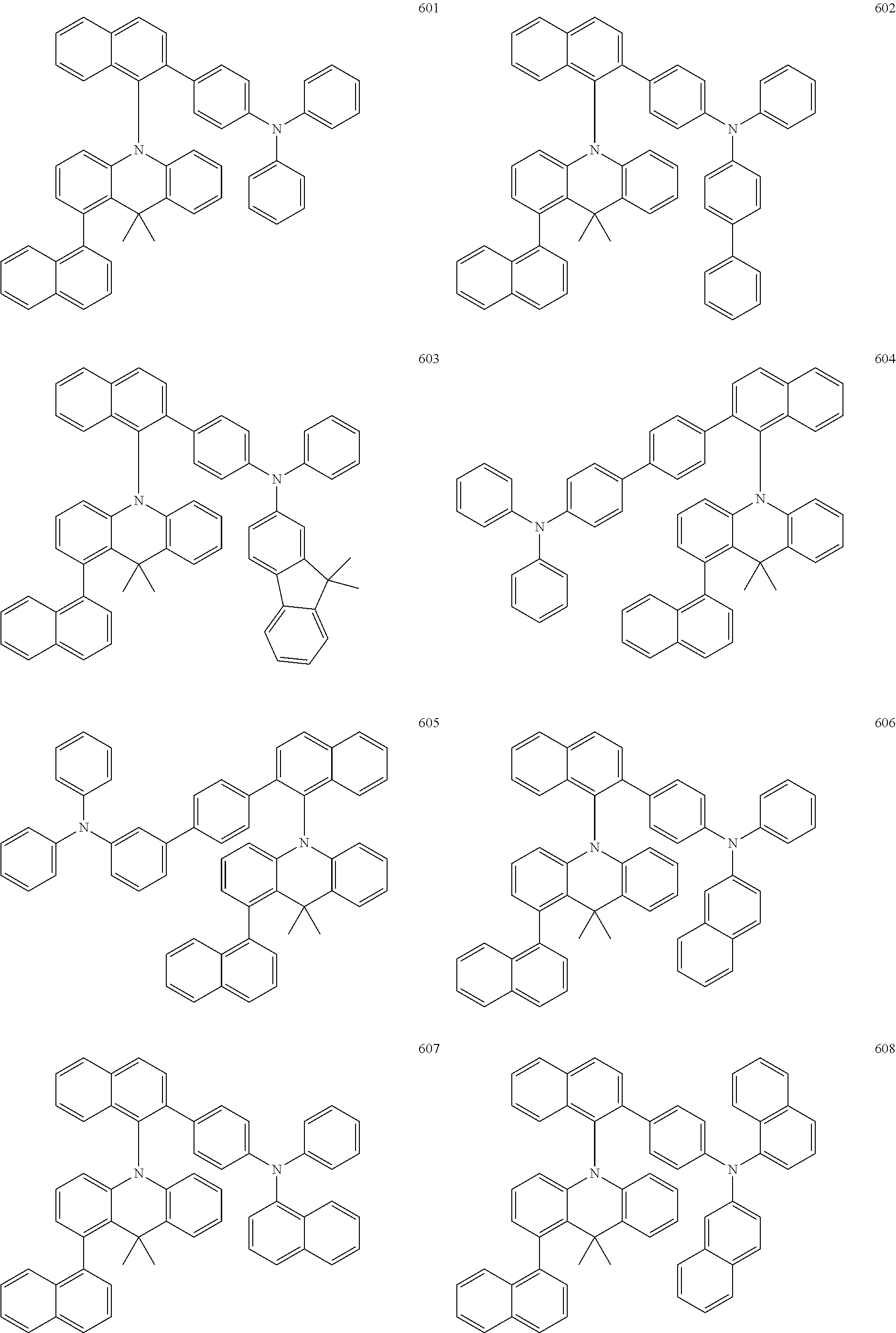

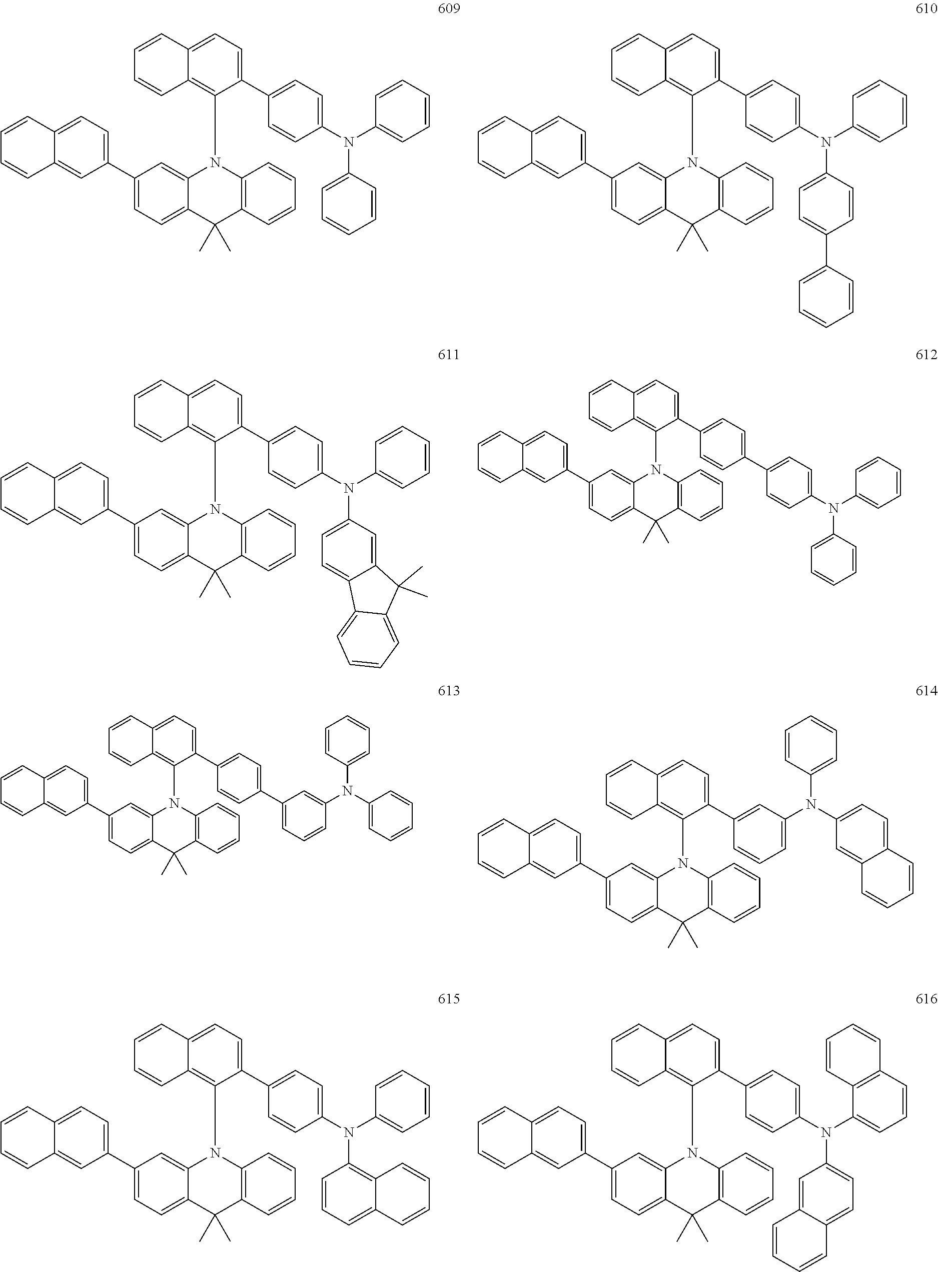

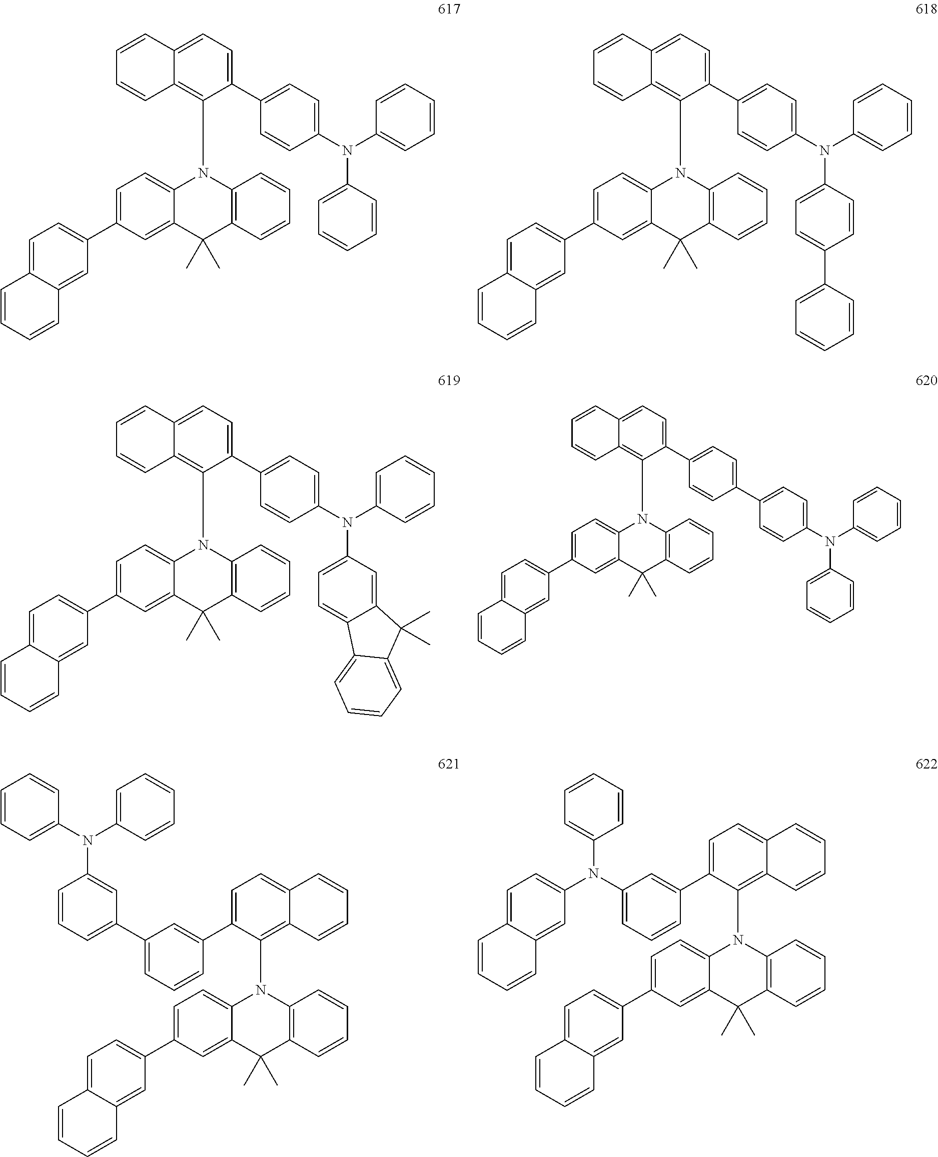

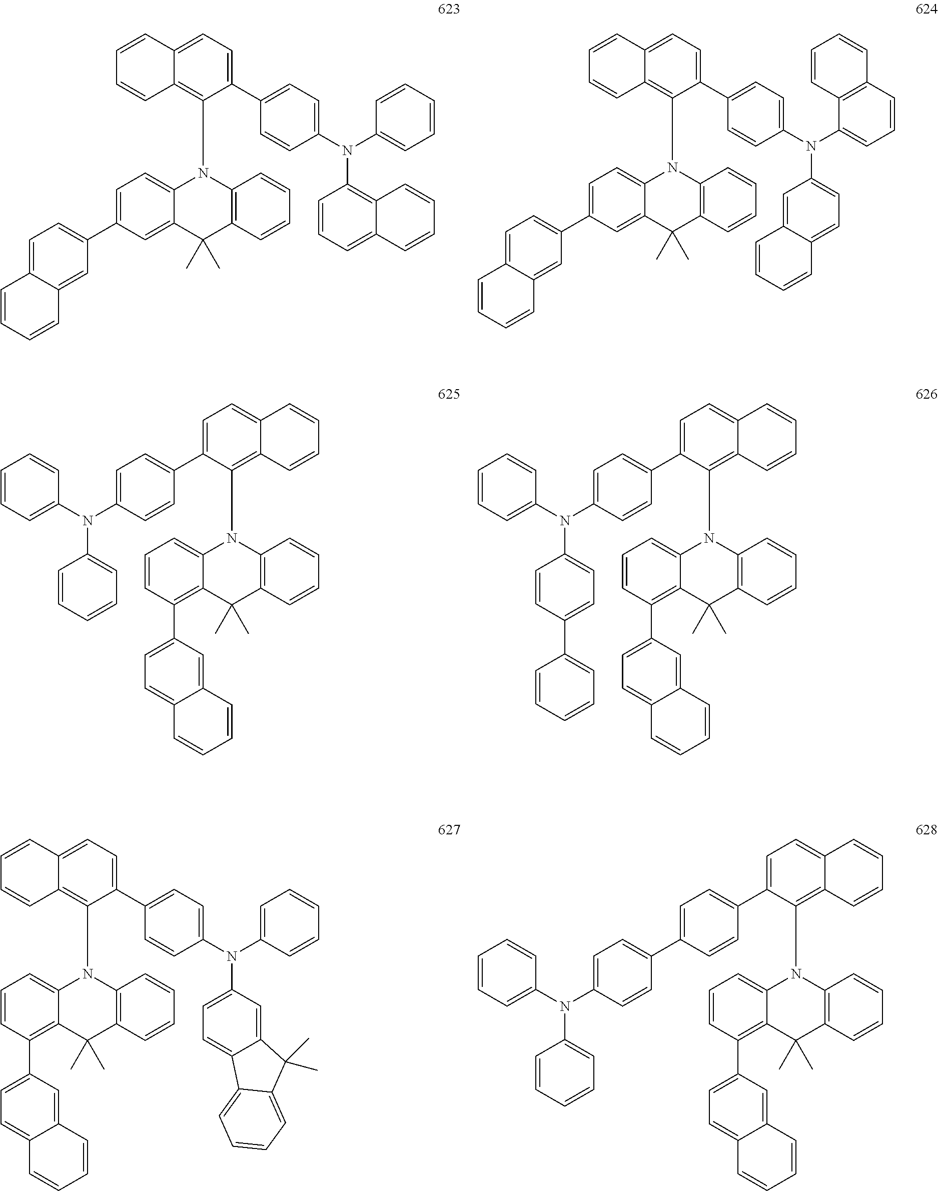

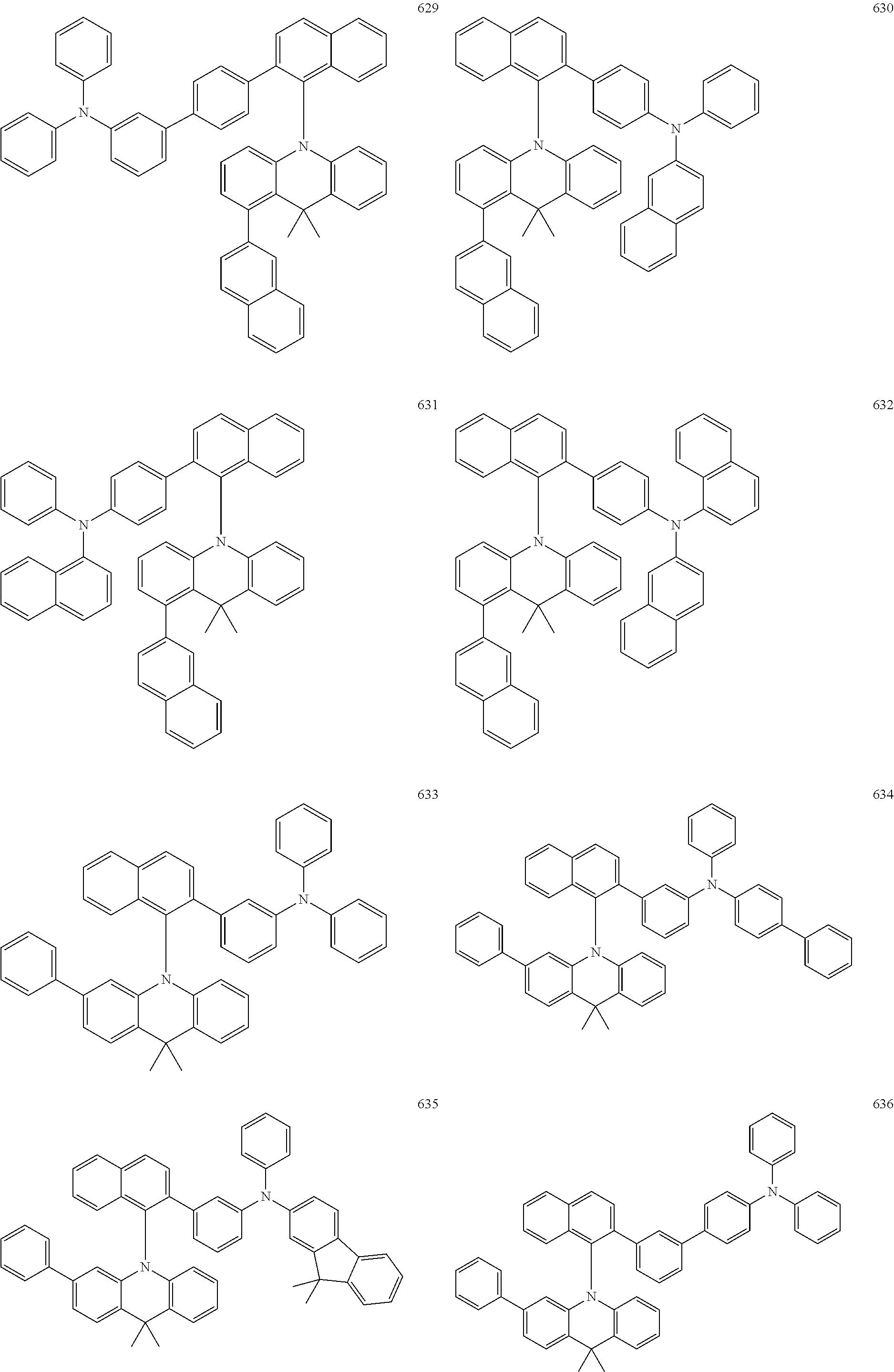

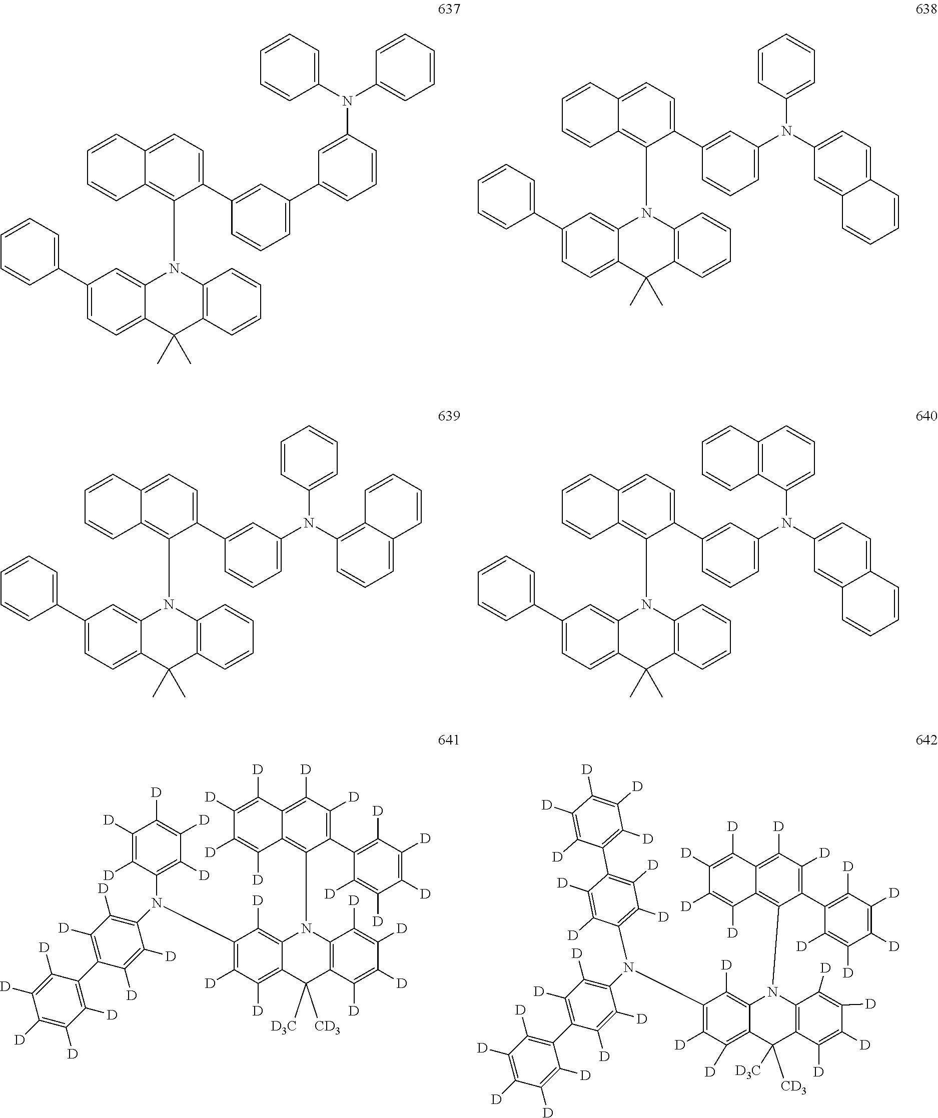

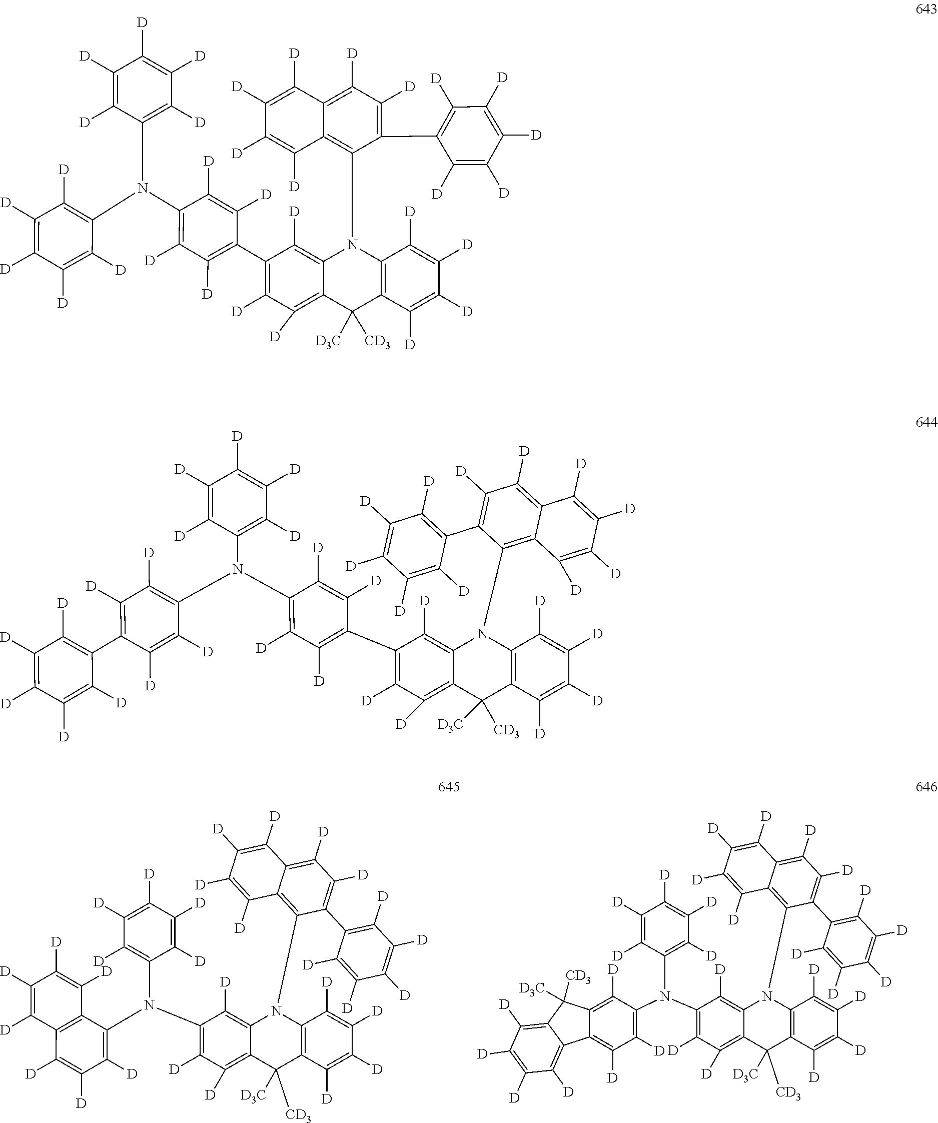

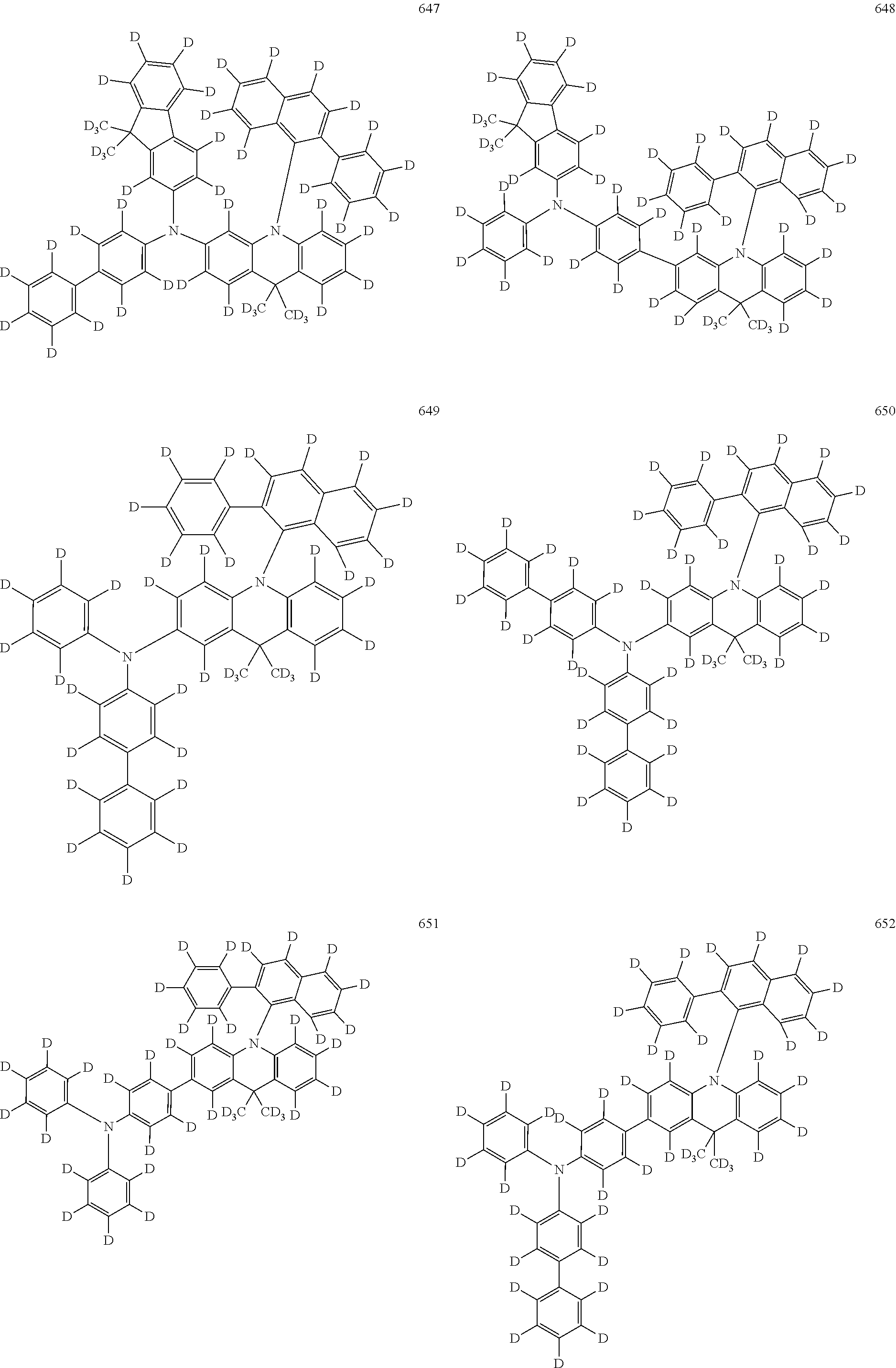

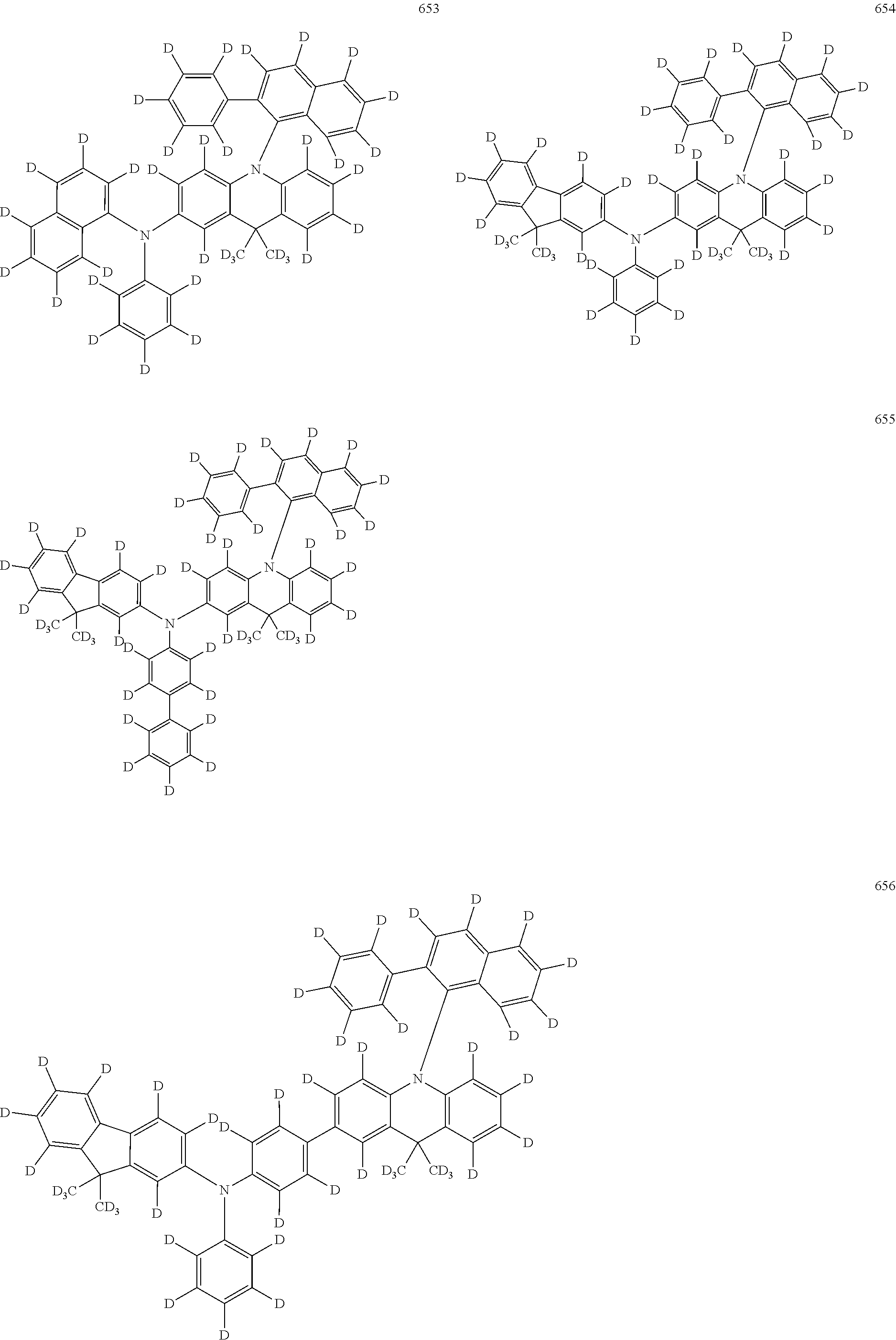

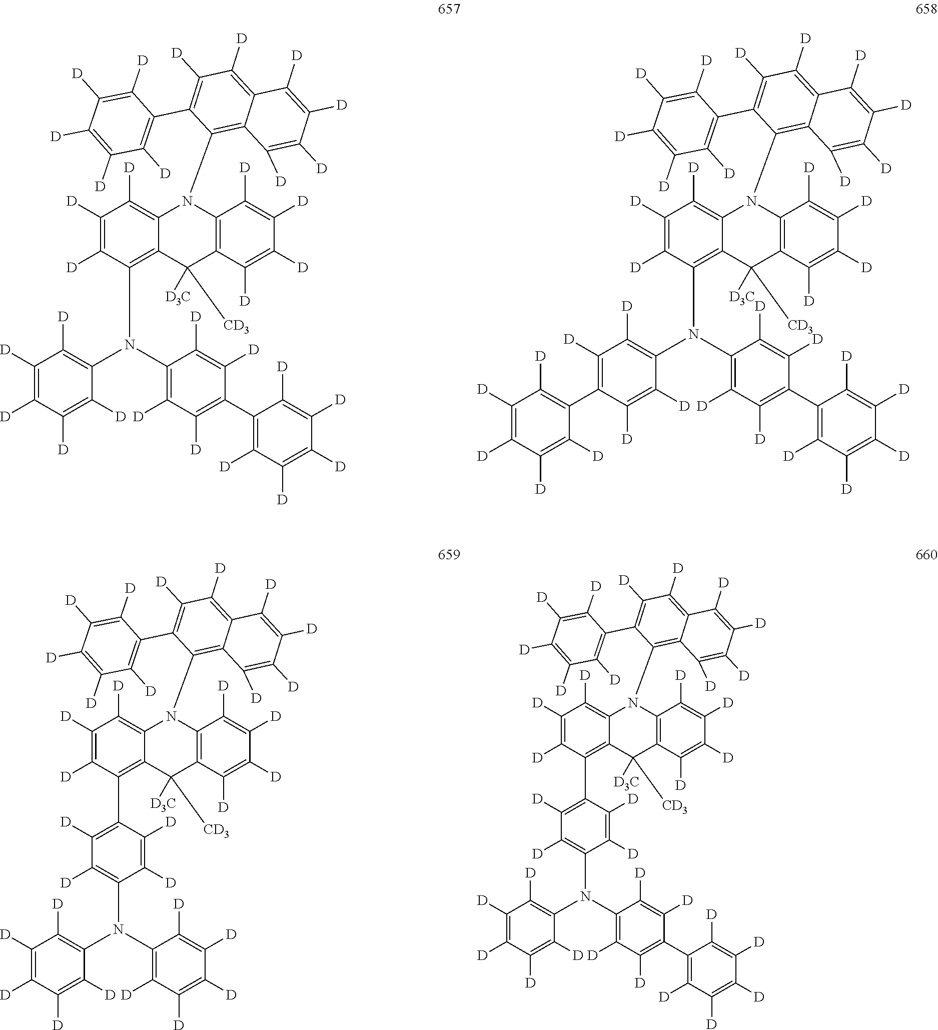

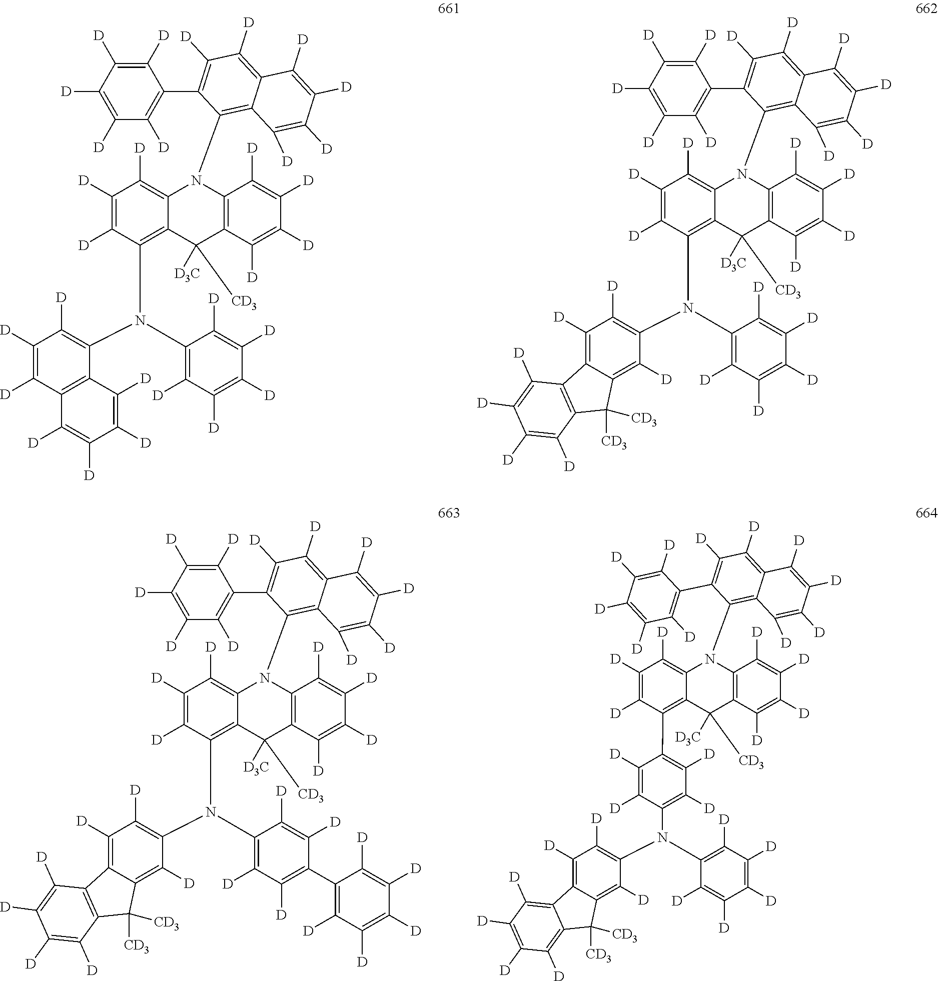

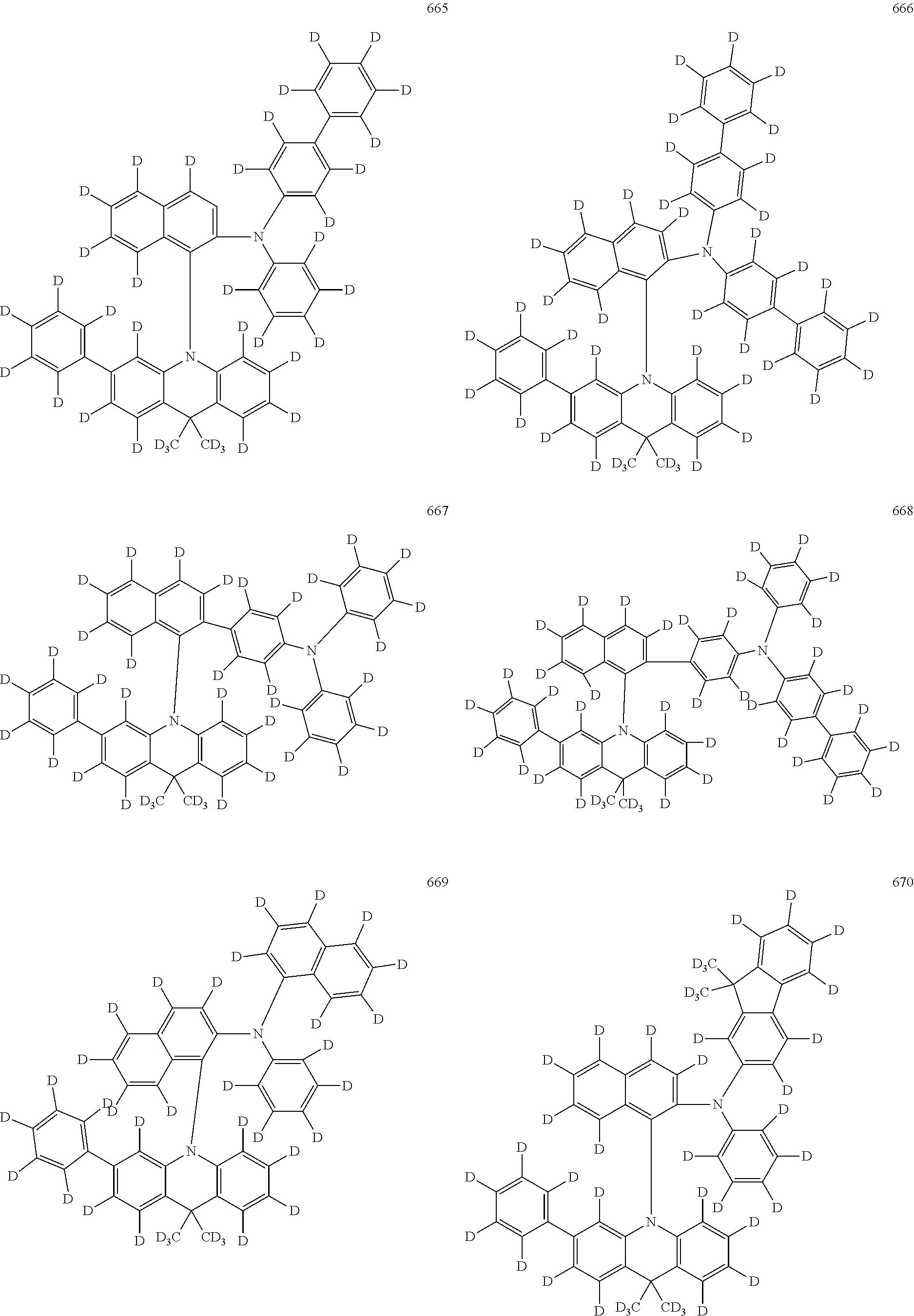

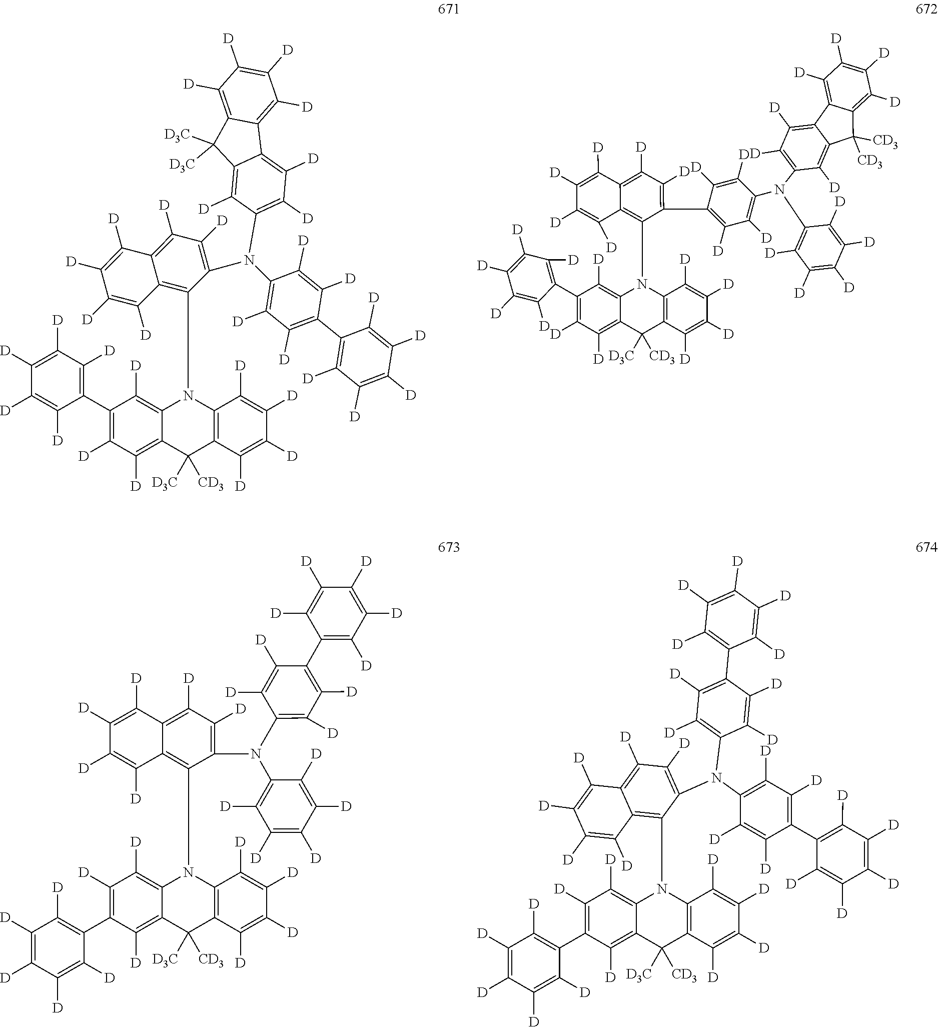

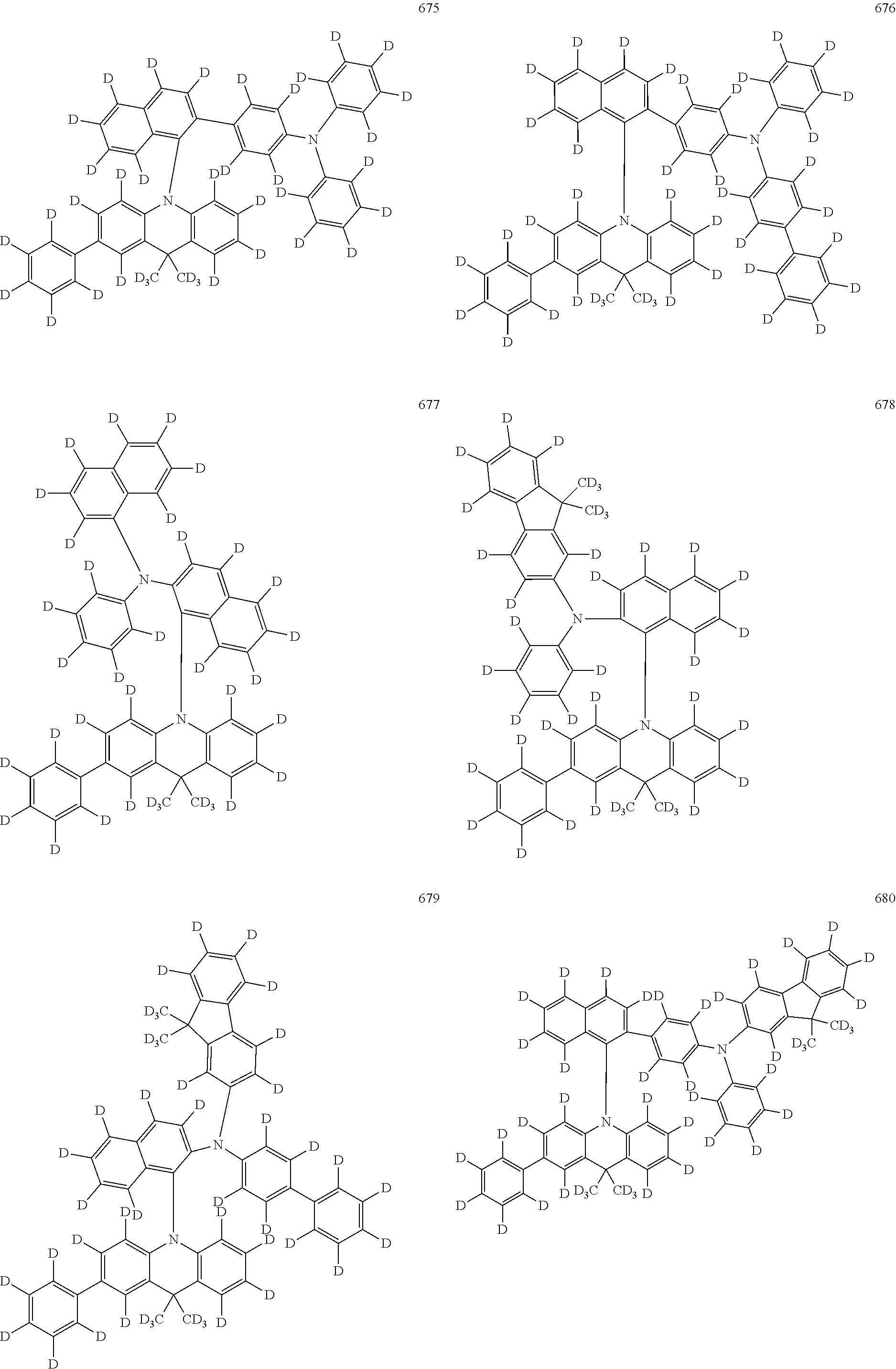

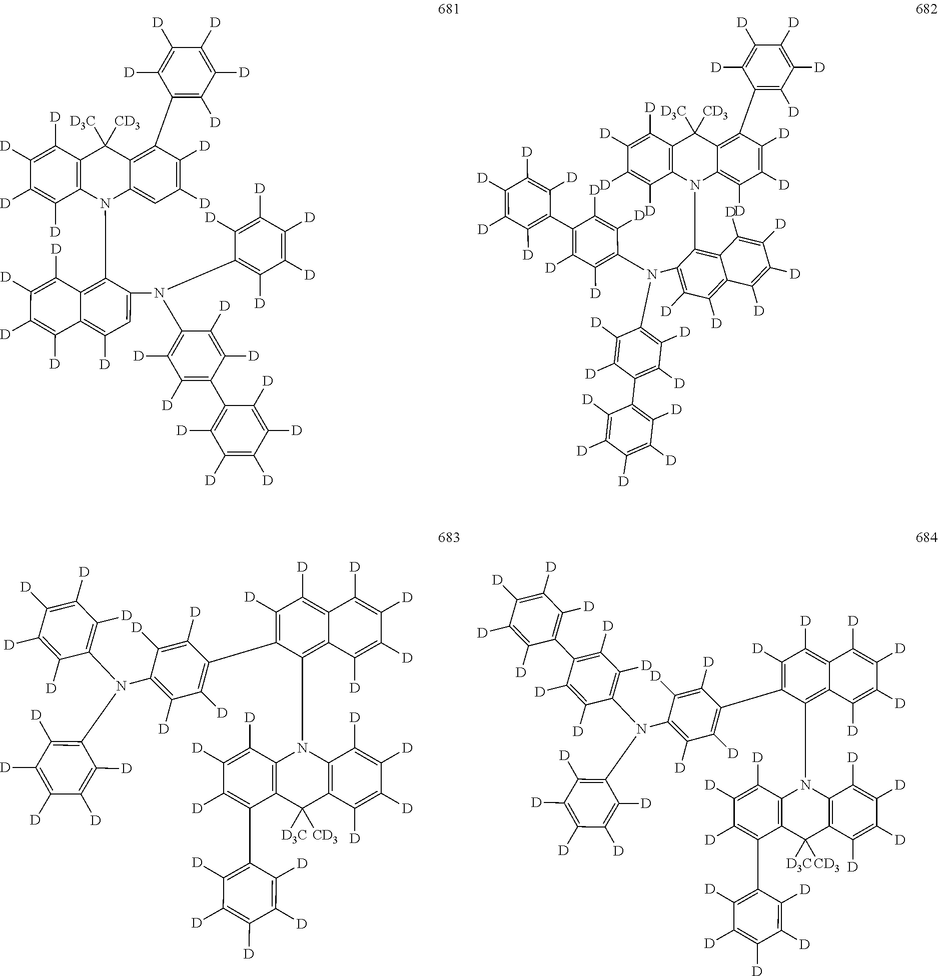

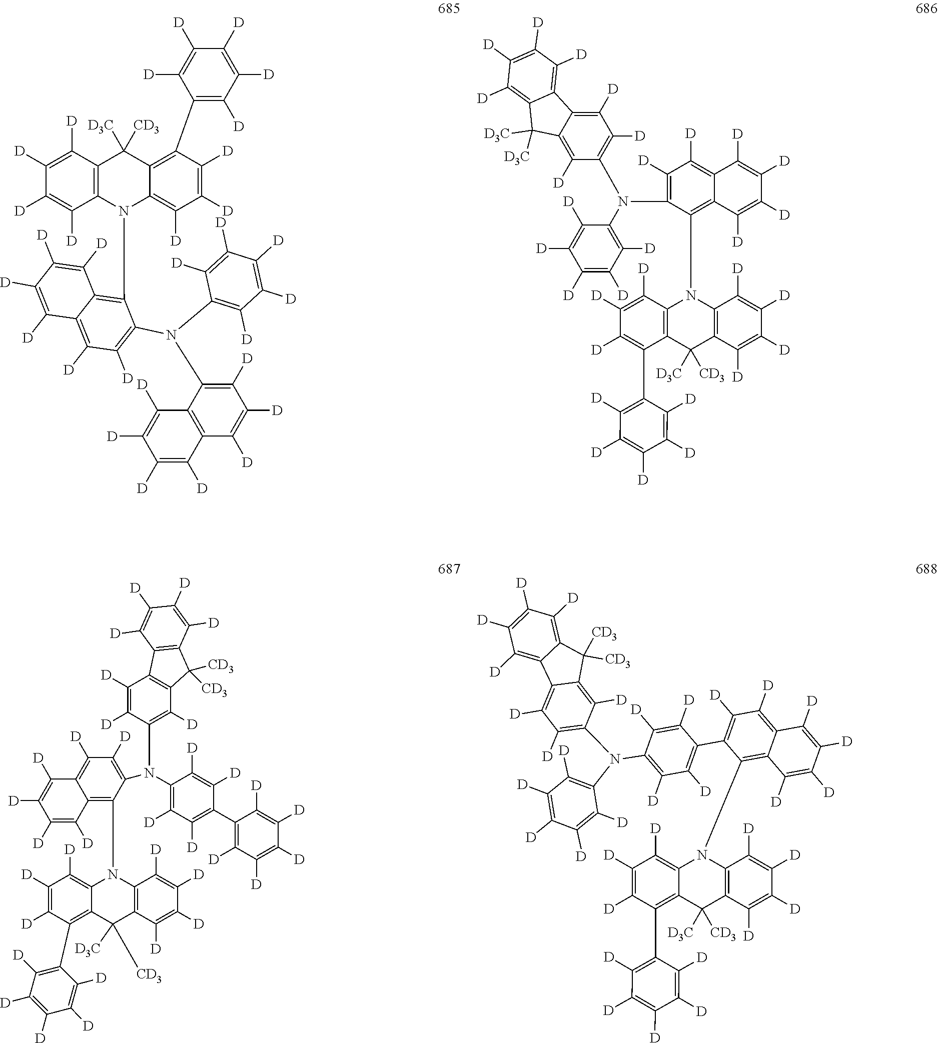

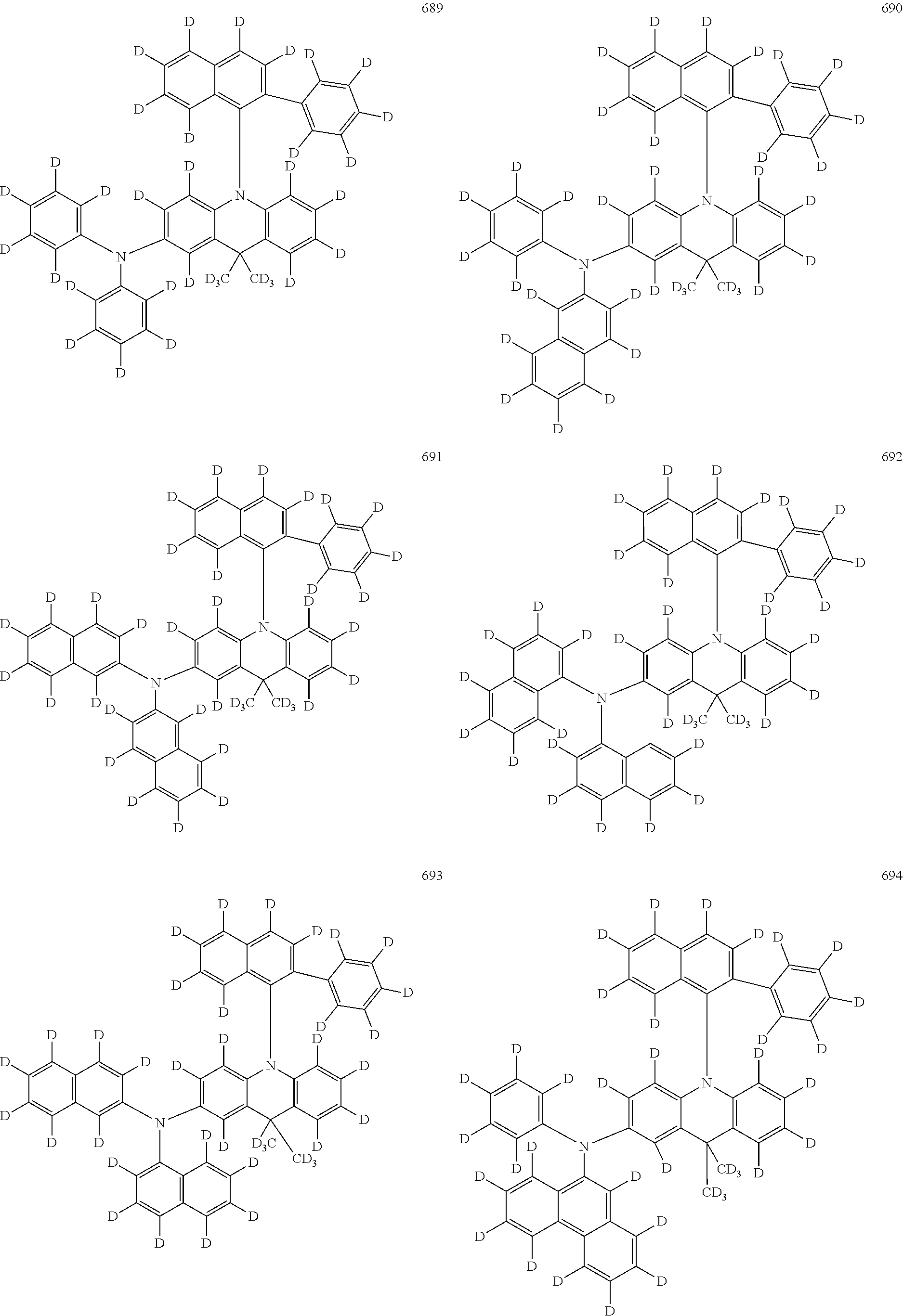

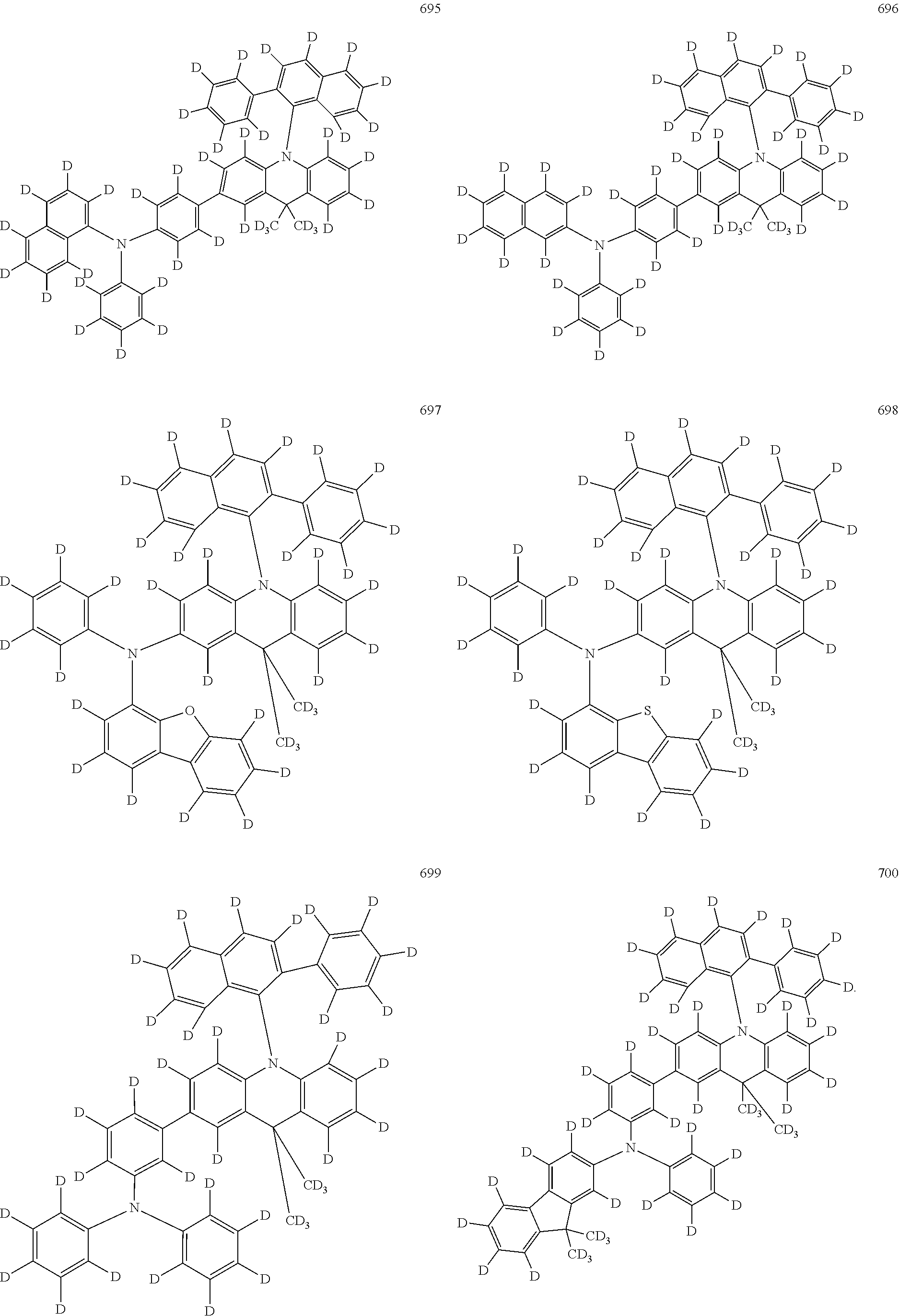

7. An organic light emitting device comprising:
a first electrode;
a second electrode provided to face the first electrode; and
one or more organic material layers provided between the first electrode and the second electrode,
wherein at least one of the one or more organic material layers comprise the heterocyclic compound according to claim 1.

8. The organic light emitting device of claim 7, further comprising one or more layers selected from the group consisting of a light emitting layer, a hole injection layer, a hole transport layer, an electron injection layer, an electron transport layer, an electron blocking layer, and a hole blocking layer.
